(19) United States
(12) Patent Application Publication
(10) Pub. No.: US 2026/0295067 A1
(43) Pub. Date: Oct. 1, 2026

(54) PANRAS INHIBITOR ANTIBODY-DRUG CONJUGATES AND METHODS OF USE THEREOF

(71) Applicant: NOVARTIS AG, Basel (CH)

(72) Inventors: Matthew T. BURGER, Belmont, MA (US); Joseph Anthony D'ALESSIO, Cambridge, MA (US); Kathleen Tian XIE, Cambridge, MA (US)

(21) Appl. No.: 19/164,169

(22) PCT Filed: Mar. 7, 2024
(86) PCT No.: PCT/IB2024/052221

§ 371 Date: Sep. 10, 2025

Related U.S. Application Data
(60) Provisional application No. 63/451,458, filed on Mar. 10, 2023.

(51) Int. Cl. A61K 47/68; A61P 35/00; G01N 33/50

(52) U.S. Cl. CPC A61K 47/6889; A61K 47/6803; A61K 47/6851; A61P 35/00; G01N 33/5011

(57) ABSTRACT

Antibody-drug conjugates that bind to human oncology targets are disclosed. The antibody-drug conjugates comprise a panRAS inhibitor drug moiety. The disclosure further relates to methods and compositions for use in the treatment of cancers by administering the antibody-drug conjugates provided herein. Linker-drug conjugates comprising panRAS inhibitor drug moiety and methods of making same are also disclosed.

### FIELD OF THE INVENTION

The present disclosure relates to antibody-drug conjugates (ADCs) comprising a panRAS inhibitor and an antibody or antigen-binding fragment thereof that binds an antigen target, e.g., an antigen expressed on a tumor or other cancer cells. The disclosure further relates to methods and compositions useful in the treatment and/or diagnosis of cancers that express a target antigen and/or are amenable to treatment by modulating panRAS expression and/or activity, as well as methods of making those compositions. Linker-drug conjugates comprising a panRAS inhibitor drug moiety and methods of making same are also disclosed.

### BACKGROUND OF THE INVENTION

Ras proteins (K-Ras, H-Ras and N-Ras) play an essential role in various human cancers and are therefore appropriate targets for anticancer therapy. Indeed, mutations in Ras proteins account for approximately 30% of all human cancers in the United States, many of which are fatal. Dysregulation of Ras proteins by activating mutations, overexpression or upstream activation is common in human tumors, and activating mutations in Ras are frequently found in human cancer. For example, activating mutations at codon 12 in Ras proteins function by inhibiting both GTPase-activating protein (GAP)-dependent and intrinsic hydrolysis rates of GTP, significantly skewing the population of Ras mutant proteins to the “on” (GTP-bound) state (Ras(ON)), leading to oncogenic MAPK signaling. Notably, Ras exhibits a picomolar affinity for GTP, enabling Ras to be activated even in the presence of low concentrations of this nucleotide. Mutations at codons 13 (e.g., G13D) and 61 (e.g., Q61K) of Ras are also responsible for oncogenic activity in some cancers.

Despite extensive drug discovery efforts against Ras during the last several decades, additional efforts are needed to uncover additional medicines for cancers driven by the various Ras mutations.

### SUMMARY OF THE INVENTION

In some embodiments, the present disclosure provides, in part, novel antibody-drug conjugate (ADC) compounds with biological activity against cancer cells. The compounds may slow, inhibit, and/or reverse tumor growth in mammals, and/or may be useful for treating human cancer patients. The present disclosure more specifically relates, in some embodiments, to ADC compounds that are capable of binding and killing cancer cells. In some embodiments, the ADC compounds disclosed herein comprise a conjugate linker that attaches a panRAS inhibitor to a full-length antibody or an antigen-binding fragment. In some embodiments, the ADC compounds are also capable of internalizing into a target cell after binding.

In some embodiments, ADC compounds may be represented by Formula (1):

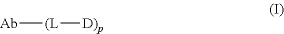

wherein Ab is an antibody or an antigen-binding fragment thereof;
D is a panRAS inhibitor;
L is a conjugate linker that covalently attaches Ab to D; and
p is an integer from 1 to 16. In some embodiments, Ab is an antibody or an antigen-binding fragment thereof that targets a cancer cell.

In some embodiments, for ADC compounds of Formula (1), D comprises a panRAS inhibitor compound of Formula (Ia) covalently attached to the conjugate linker L:

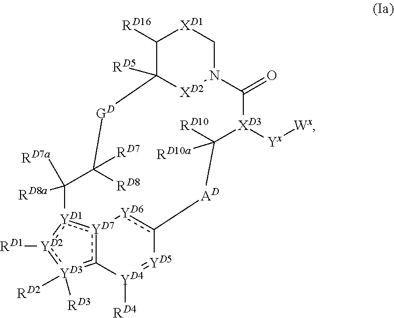

or
a pharmaceutically acceptable salt thereof, wherein:
the dotted lines represent zero, one, two, three, or four non-adjacent double bonds;
A^{D} is —N(H or CH₃)C(O)—(CH₂)— where the amino nitrogen is bound to the carbon atom of —C(R^{D10a})(R^{D10})—, optionally substituted 3 to 6-membered cycloalkylene, optionally substituted 3 to 6-membered heterocycloalkylene, optionally substituted 6-membered arylene, or optionally substituted 5 to 6-membered heteroarylene;
Y^{X} is

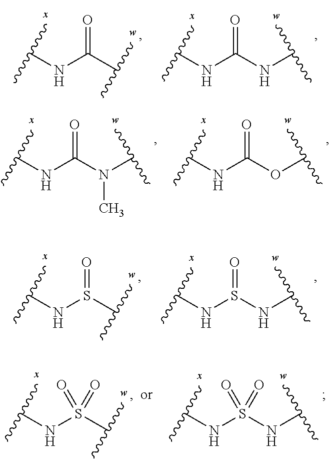

wherein

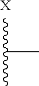

indicates the point that connects with X^{D3}; and

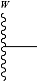

indicates the point that connects with W^{X}; or
Y^{X} is —N(R^{D11})—CO—B^{D}-L^{D}:
B^{D} is —CH(R^{D9})— or >C═CR^{D9}R^{D9′} where the carbon is bound to the carbonyl carbon of —N(R^{D11})C(O)—, optionally substituted 3 to 6-membered cycloalkylene, optionally substituted 3 to 6-membered heterocycloalkylene, optionally substituted 6-membered arylene, or 5 to 6-membered heteroarylene;
L^{D} is absent or a linker;
G^{D} is optionally substituted C₁-C₄ alkylene, optionally substituted C₁-C₄ alkenylene, optionally substituted C₁-C₄ heteroalkylene, —C(O)O—CH(R^{D6})— where —CH(R^{D6})— is bound to —C(R^{D7}R^{D8})—, —C(O)NH—CH(R^{D6})— where —CH(R^{D6})— is bound to —C(R^{D7}R^{D8})—, optionally substituted C₁-C₄ heteroalkylene, or 3 to 8-membered heteroarylene;
W^{X} is hydrogen, cyano, optionally substituted C₁-C₃ heteroalkyl, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, optionally substituted 6 to 10-membered aryl, or optionally substituted 3 to 8-membered heteroaryl;
X^{D1} is optionally substituted C₁-C² alkylene, NR^{D}, O, or S(O)_{nD};
X^{D2} is O or NH;
X^{D3} is N or CH;
n^{D} is 0, 1, or 2;
R^{D} is hydrogen, cyano, optionally substituted C₁-C₄ alkyl, optionally substituted C₂-C₄ alkenyl, optionally substituted C₂-C₄ alkynyl, C(O)R^{D′}, C(O)OR^{D′}, C(O)N(R^{D′})₂, S(O)R^{D′}, S(O)₂R^{D′}, or S(O)₂N(R^{D′})₂; each R^{D′} is, independently, H or optionally substituted C₁-C₄ alkyl;
Y^{D1} is C, CH, or N;
Y^{D2}, Y^{D3}, Y^{D4} and Y^{D7} are, independently, C or N;
Y^{D5} is CH, CH₂, or N;
Y^{D6} is C(O), CH, CH₂, or N;
R^{D1} is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R^{D1} and R^{D2} combine with the atoms to which they are attached to form an optionally substituted 3 to 14-membered heterocycloalkyl;
R^{D2} is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R^{D3} is absent or R^{D2} and R^{D3} combine with the atom to which they are attached to form an optionally substituted 3 to 8-membered cycloalkyl or optionally substituted 3 to 14-membered heterocycloalkyl;
R^{D4} is absent, hydrogen, halogen, cyano, or methyl optionally substituted with 1 to 3 halogens;
R^{D5} is hydrogen, C₁-C₄ alkyl optionally substituted with halogen, cyano, hydroxy, or C₁-C₄ alkoxy, cyclopropyl, or cyclobutyl;
R^{D6} is hydrogen or methyl;
R^{D7} is hydrogen, halogen, or optionally substituted C₁-C₃ alkyl, or
R^{D6} and R^{D7} combine with the carbon atoms to which they are attached to form an optionally substituted 3 to 6-membered cycloalkyl or optionally substituted 3 to 7-membered heterocycloalkyl;
R^{D8} is hydrogen, halogen, hydroxy, cyano, optionally substituted 01-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{D7} and R^{D8} combine with the carbon atom to which they are attached to form C═CR^{D7′}R^{D8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{D7a} and R^{D8a} are, independently, hydrogen, halo, optionally substituted C₁-C₃ alkyl, or combine with the carbon to which they are attached to form a carbonyl;
R^{D7′} is hydrogen, halogen, or optionally substituted C₁-C₃ alkyl;
R^{D8′} is hydrogen, halogen, hydroxyl, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{D7′} and R^{D8′} combine with the carbon atom to which they are attached to form optionally substituted 3 to 6-membered cycloalkyl or optionally substituted 3 to 7-membered heterocycloalkyl;
R^{D9} is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R^{D9} and L^{D} combine with the atoms to which they are attached to form an optionally substituted 3 to 14-membered heterocycloalkyl;
R^{D9′} is hydrogen or optionally substituted C₁₋₆ alkyl;
R^{D10} is hydrogen, halo, hydroxyl, C₁-C₃ alkoxyl, or C₁-C₃ alkyl;
R^{D10a} is hydrogen or halogen;
R^{D11} is hydrogen or C₁-C₃ alkyl; and
R^{D16} is hydrogen or C₁-C₃ alkyl.

In some embodiments, p is an integer from 1 to 8. In some embodiments, p is an integer from 1 to 6. In some embodiments, p is an integer from 1 to 5. In some embodiments, p is an integer from 2 to 4. In some embodiments, p is 1. In some embodiments, p is 2. In some embodiments, p is 4. In some embodiments, p is determined by liquid chromatography-mass spectrometry (LC-MS).

In some embodiments, the conjugate linker (L) comprises an attachment group, at least one spacer group, and at least one cleavable group. In some cases, the cleavable group comprises a pyrophosphate group and/or a self-immolative group. In specific embodiments, L comprises an attachment group; at least one bridging spacer group; and at least one cleavable group comprising a pyrophosphate group and/or a self-immolative group.

In some embodiments, the antibody-drug conjugate comprises a linker-drug (or “linker-payload”) moiety -(L-D) is of the formula (A):

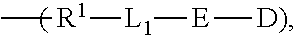

wherein R¹ is an attachment group, L₁ is a bridging spacer group, and E is a cleavable group.

In some embodiments, the cleavable group comprises a pyrophosphate group. In some embodiments, the cleavable group comprises:

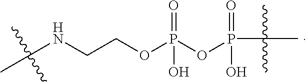 [SMILES: *NCCOP(=O)(O)OP(*)(=O)O]

In some embodiments, the bridging spacer group comprises a polyoxyethylene (PEG) group. In some cases, the PEG group may be selected from PEG1, PEG2, PEG3, PEG4, PEG5, PEG6, PEG7, PEG8, PEG9, PEG10, PEG11, PEG12, PEG13, PEG14, and PEG15. In some embodiments, the bridging spacer group may comprise: —CO—CH₂—CH₂—PEG12-. In other embodiments, the bridging spacer group comprises a butanoyl, pentanoyl, hexanoyl, heptanoyl, or octanoyl group. In some embodiments, the bridging spacer group comprises a hexanoyl group.

In some embodiments the attachment group is formed from at least one reactive group selected from a maleimide group, thiol group, cyclooctyne group, and an azido group. For example, maleimide group may have the structure:

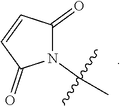 [SMILES: *N1C(=O)C=CC1=O]

The azido group may have the structure: —N═N⁺═N⁻.

The cyclooctyne group may have the structure:

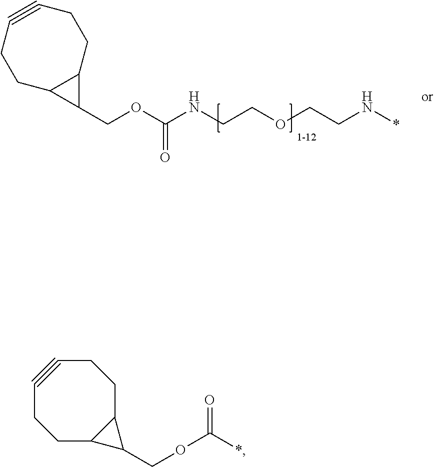

and wherein —* is a bond to the antibody or antigen-binding fragment thereof.

In some cases, the cyclooctyne group has the structure:

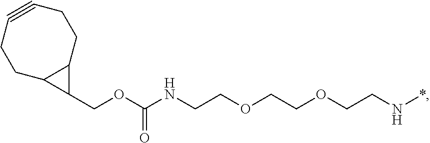

and wherein —* is a bond to the antibody or antigen-binding fragment thereof.

In some embodiments, the attachment group has a formula comprising

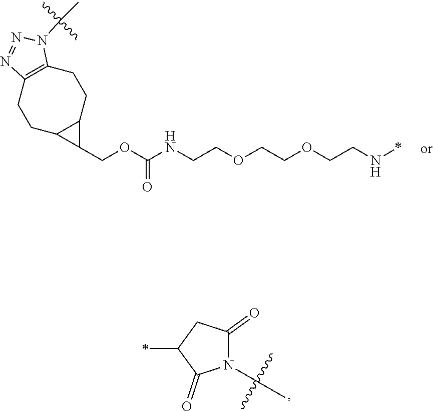 [SMILES: *NCCOCCOCCNC(=O)OCC1C2CCc3nnn(*)c3CCC21 ; *C1CC(=O)N(*)C1=O]

and wherein —* is a bond to the antibody or antigen-binding fragment thereof.

In some embodiments, the antibody or antigen-binding fragment thereof is joined to the conjugate linker (L) by an attachment group selected from:

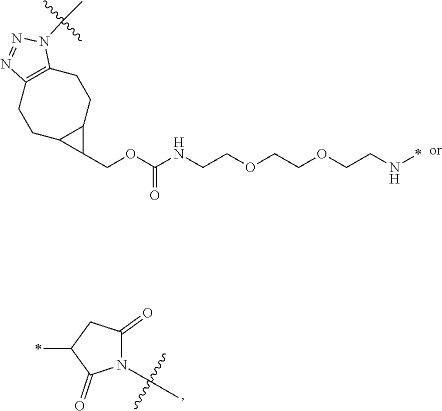 [SMILES: *NCCOCCOCCNC(=O)OCC1C2CCc3nnn(*)c3CCC21 ; *C1CC(=O)N(*)C1=O]

wherein —* is a bond to the antibody or antigen-binding fragment thereof, and wherein

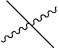

is a bond to the bridging spacer group. As used herein, the term “joined” refers to covalently attached to or covalently linked.

In some embodiments, the bridging spacer group is joined or covalently linked to a cleavable group.

In some embodiments, the bridging spacer group is —CH₂CH₂—O—CH₂CH₂—CO—.

In some embodiments, the cleavable group is -pyrophosphate-CH₂—CH₂—NH₂—.

In some embodiments, the cleavable group is joined or covalently linked to the panRAS inhibitor (D).

In some embodiments, the conjugate linker comprises: an attachment group, at least one bridging spacer group, a peptide group, and at least one cleavable group.

In some embodiments, the antibody-drug conjugate comprises a linker-drug moiety, -(L-D), is of the formula (B):

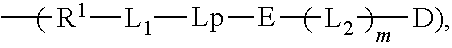

wherein R¹ is an attachment group, L₁ is a bridging spacer, Lp is a peptide group comprising 1 to 6 amino acid residues, E is a cleavable group, L₂ is a bridging spacer, m is 0 or 1; and D is a panRAS inhibitor. In some cases, m is 1 and the bridging spacer comprises:

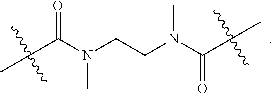 [SMILES: *C(=O)N(C)CCN(C)C(*)=O]

In some embodiments, the at least one bridging spacer comprises a PEG group. In some cases, the PEG group is selected from, PEG1, PEG2, PEG3, PEG4, PEG5, PEG6, PEG7, PEG8, PEG9, PEG10, PEG11, PEG12, PEG13, PEG14, and PEG15. In some cases, the at least one bridging spacer is selected from *—C(O)—CH₂—CH₂—PEG1-**, *—C(O)—CH₂—PEG3-**, *—C(O)—CH₂—CH₂—PEG12**, *—NH—CH₂—CH₂—PEG1-**, a polyhydroxyalkyl group, *—C(O)—N(CH₃)—CH₂—CH₂—N(CH₃)—C(O)—**, *—C(O)—CH₂—CH₂—PEG12-NH—C(O)CH₂—CH₂—**, and wherein ** indicates the point of direct or indirect attachment of the at least one bridging spacer to the attachment group and * indicates the point of direct or indirect attachment of the at least one bridging spacer to the peptide group.

In some embodiments, L₁ is selected from *—C(O)—CH₂—CH₂—PEG1-**, *—C(O)—CH₂—PEG3-**, *—C(O)—CH₂—CH₂—PEG12**, *—NH—CH₂—CH₂—PEG1-**, and a polyhydroxyalkyl group, wherein ** indicates the point of direct or indirect attachment of L₁ to R¹ and * indicates the point of direct or indirect attachment of L₁ to Lp.

In some embodiments, m is 1 and L₂ is —C(O)—N(CH₃)—CH₂—CH₂—N(CH₃)—C(O)—.

In some embodiments, the peptide group comprises 1 to 12 amino acid residues. In some embodiments, the peptide group (Lp) comprises 1 to 10 amino acid residues. In some embodiments, the peptide group (Lp) comprises 1 to 8 amino acid residues. In some embodiments, the peptide group (Lp) comprises 1 to 6 amino acid residues. In some embodiments, the peptide group comprises 1 to 4 amino acid residues. In some embodiments, the peptide group comprises 1 to 3 amino acid residues. In some embodiments the peptide group comprises 1 to 2 amino acid residues. In some cases, the amino acid residues are selected from L-glycine (Gly), L-valine (Val), L-citrulline (Cit), L-cysteic acid (sulfo-Ala), L-lysine (Lys), L-isoleucine (lie), L-phenylalanine (Phe), L-methionine (Met), L-asparagine (Asn), L-proline (Pro), L-alanine (Ala), L-leucine (Leu), L-tryptophan (Trp), and L-tyrosine (Tyr). For example, the peptide group may comprise Val-Cit, Val-Ala, Val-Lys, and/or sulfo-Ala-Val-Ala. In some embodiments, the peptide group (Lp) comprises 1 amino acid residue linked to a

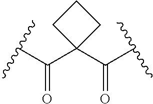 [SMILES: *C(=O)C1(C(*)=O)CCC1]

group. In some embodiments, the peptide group (Lp) comprises a group:

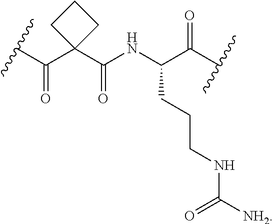 [SMILES: *C(=O)[C@H](CCCNC(N)=O)NC(=O)C1(C(*)=O)CCC1]

In some cases, the peptide group comprises a group selected from:

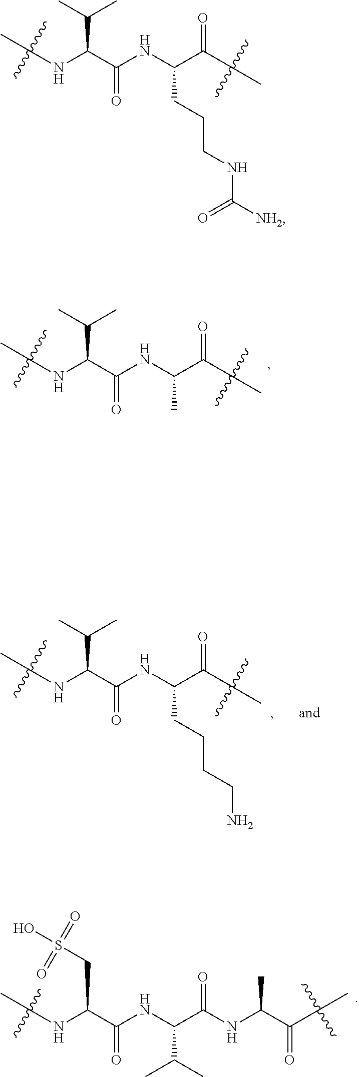 [SMILES: *N[C@H](C(=O)N[C@@H](CCCNC(N)=O)C(*)=O)C(C)C ; *N[C@H](C(=O)N[C@@H](C)C(*)=O)C(C)C ; *N[C@H](C(=O)N[C@@H](CCCCN)C(*)=O)C(C)C ; *N[C@@H](CS(=O)(=O)O)C(=O)N[C@H](C(=O)N[C@@H](C)C(*)=O)C(C)C]

In some embodiments, the self-immolative group comprises para-aminobenzyl-carbamate, para-aminobenzyl-ammonium, para-amino-(sulfo)benzyl-ammonium, para-amino-(sulfo)benzyl-carbamate, para-amino-(alkoxy-PEG-alkyl)benzyl-carbamate, para-amino-(polyhydroxycarboxytetrahydropyranyl)alkyl-benzyl-carbamate, or para-amino-(polyhydroxycarboxytetrahydropyranyl)alkyl-benzyl-ammonium.

In some embodiments, m is 1 and the bridging spacer comprises

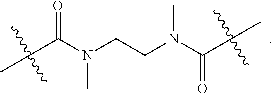 [SMILES: *C(=O)N(C)CCN(C)C(*)=O]

In some embodiments, the linker-drug moiety, -(L-D), is formed from a compound selected from:

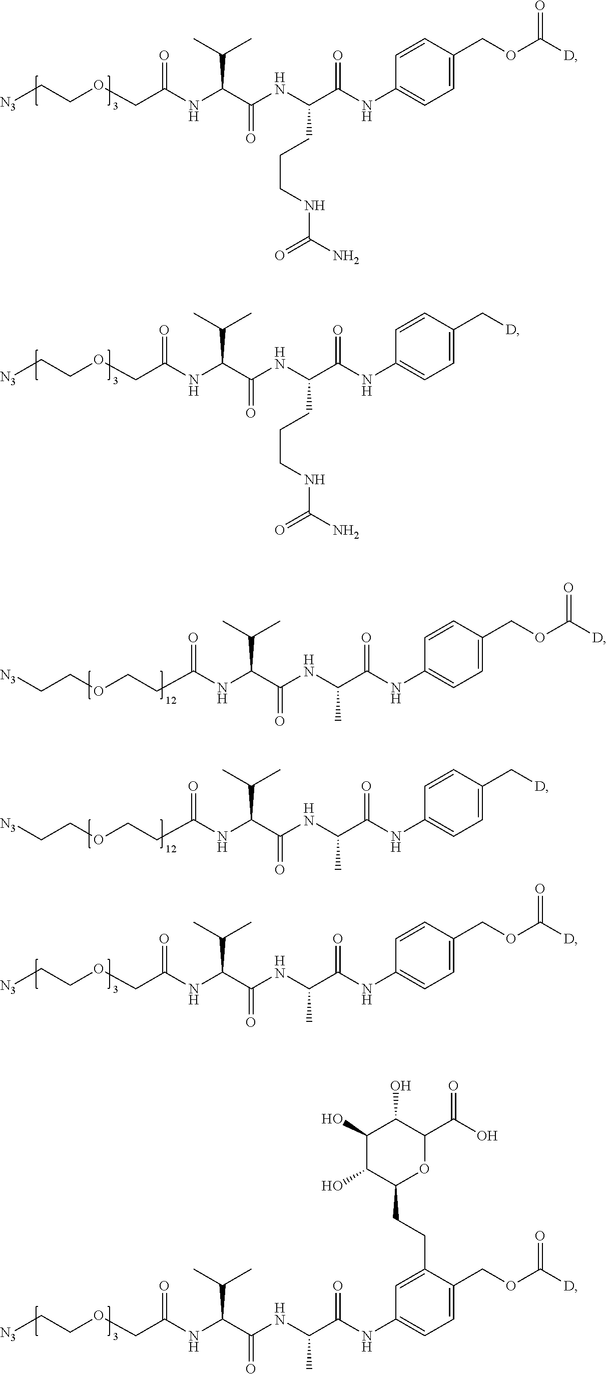

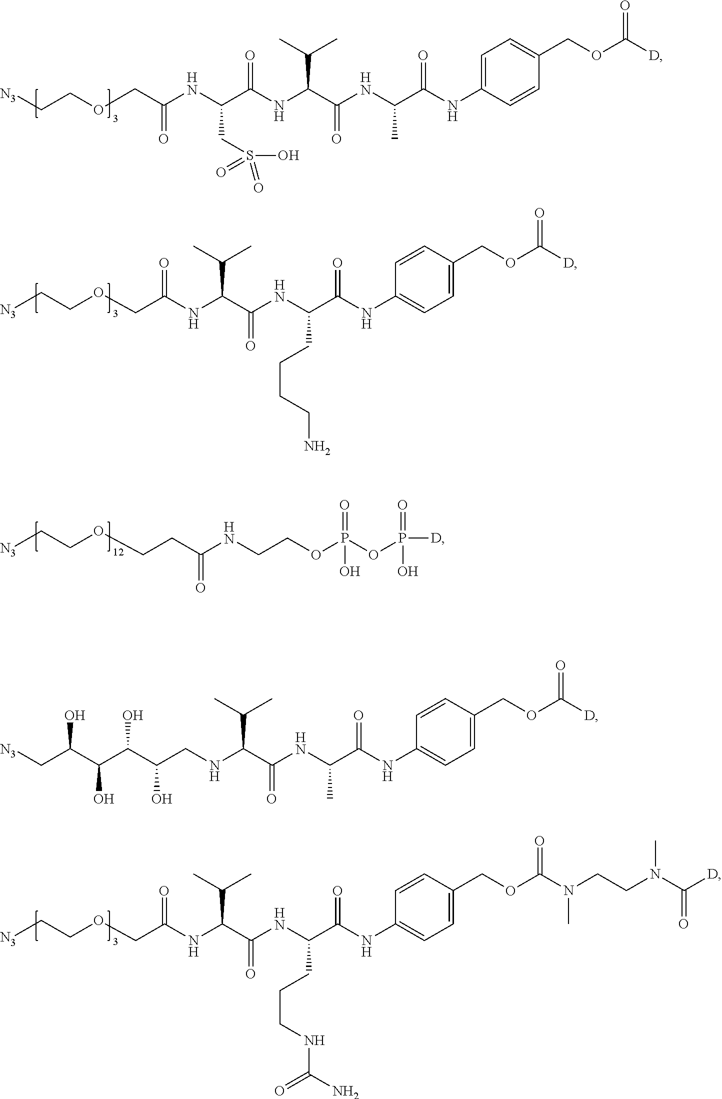

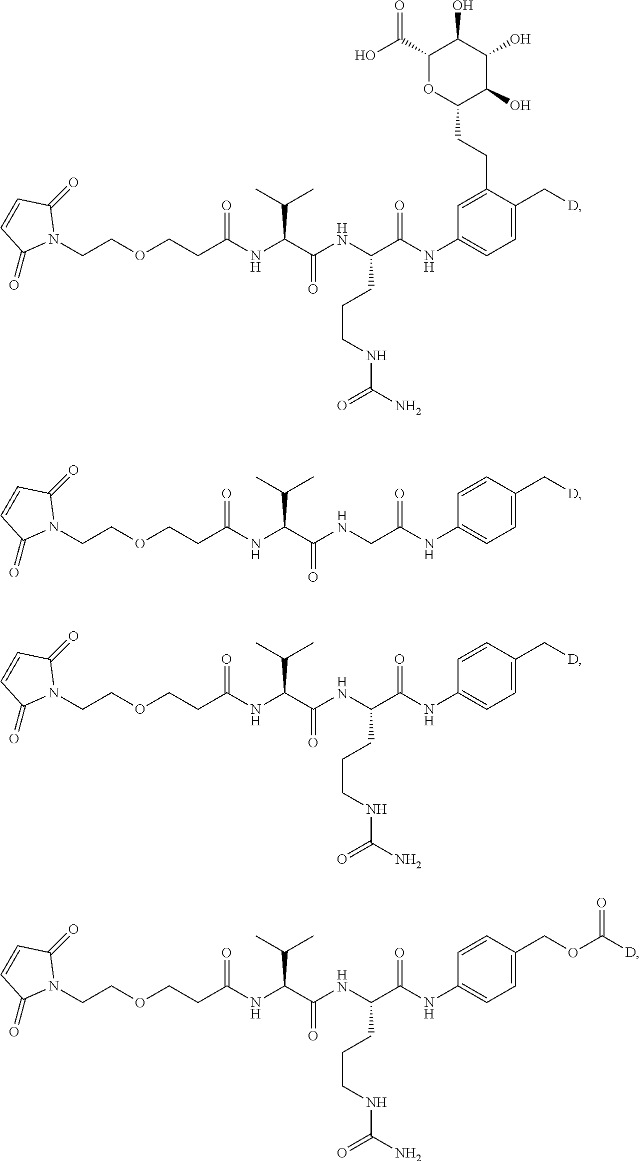

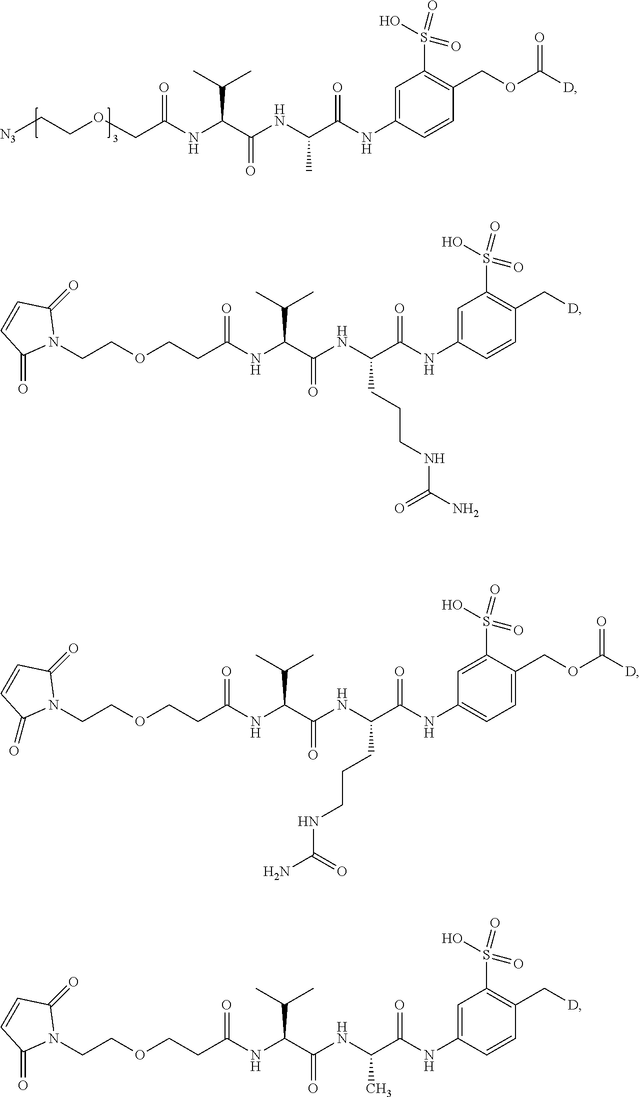

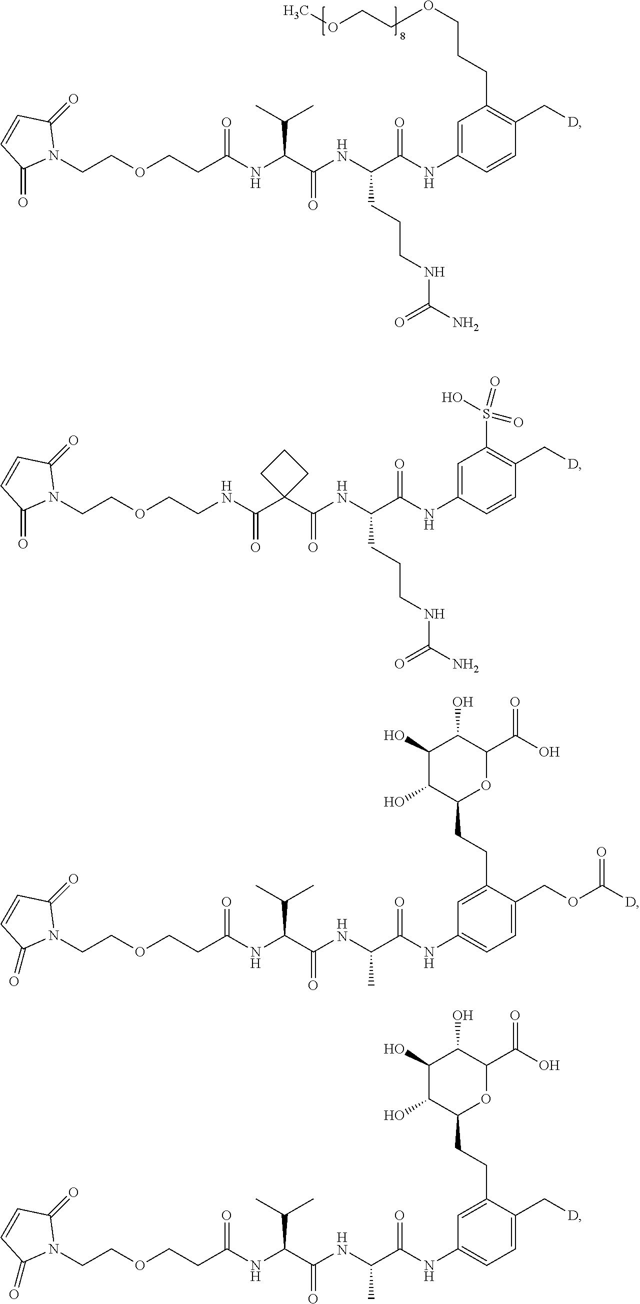

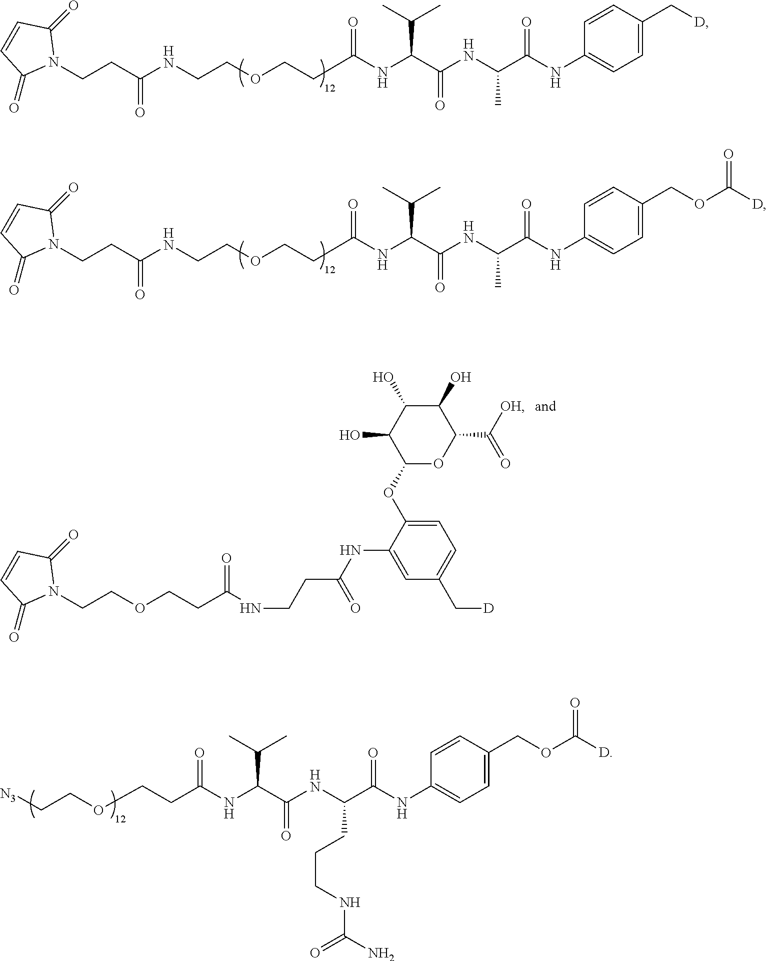

In some embodiments, the antibody-drug conjugate comprises the linker-drug group, -(L-D), which comprises a formula selected from:

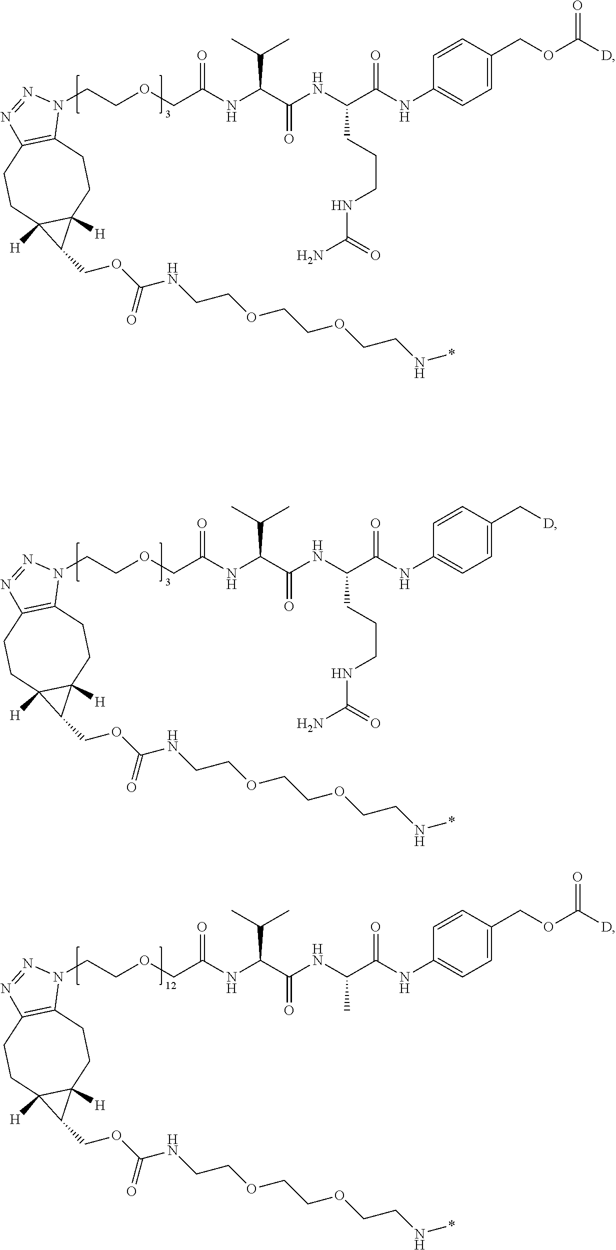

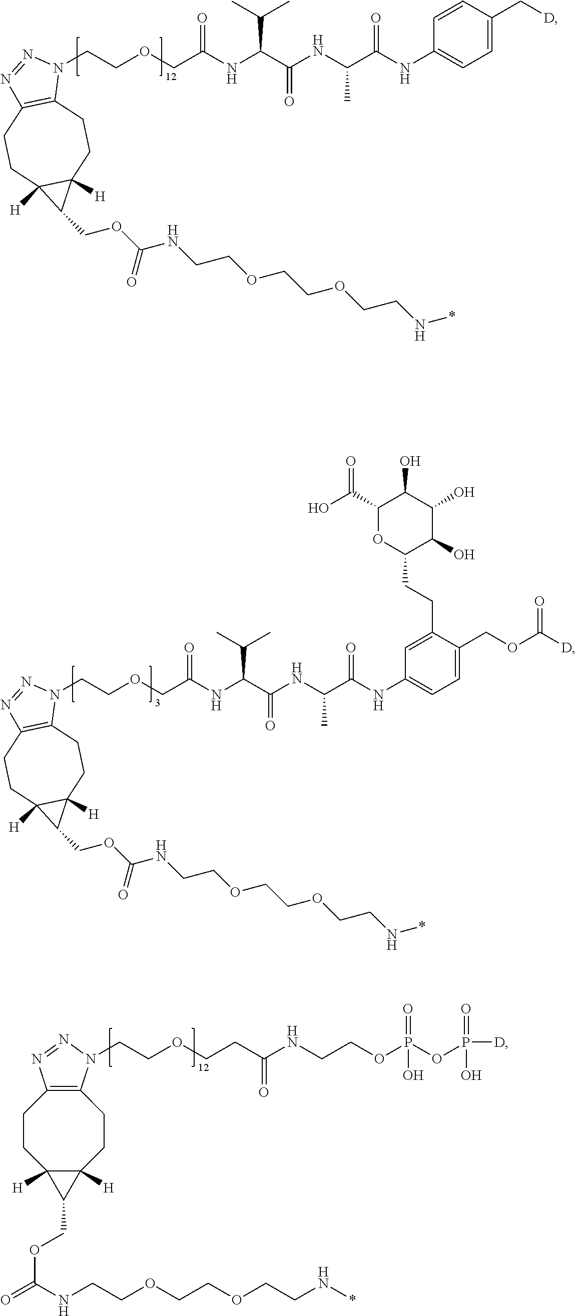

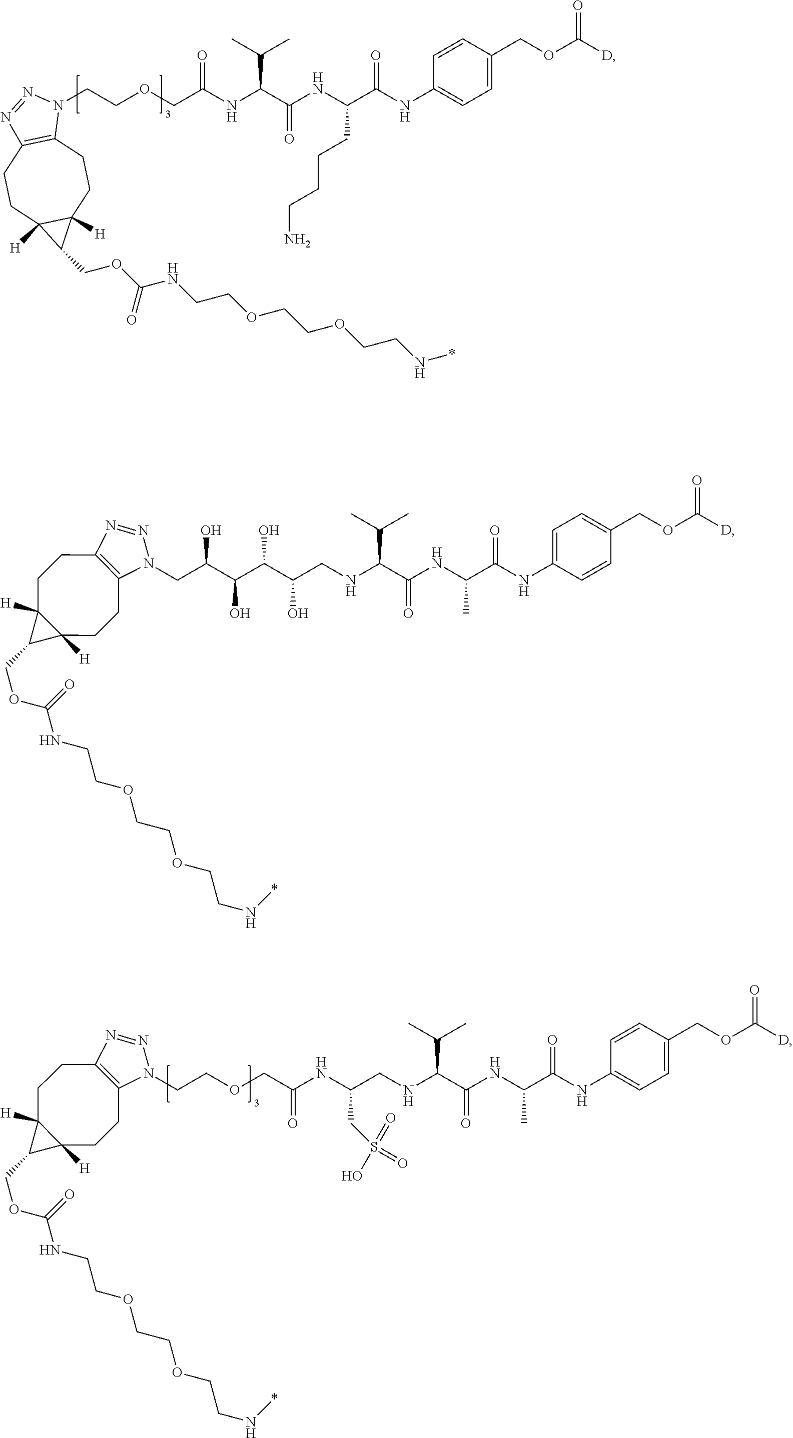

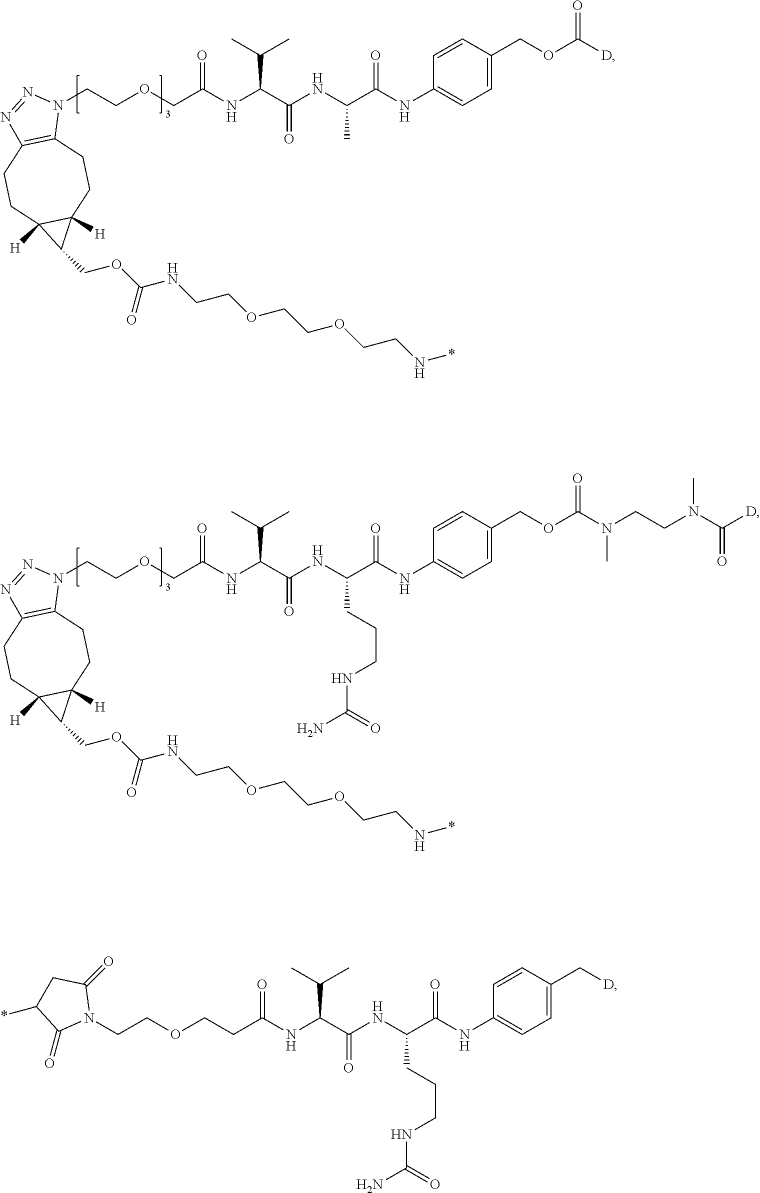

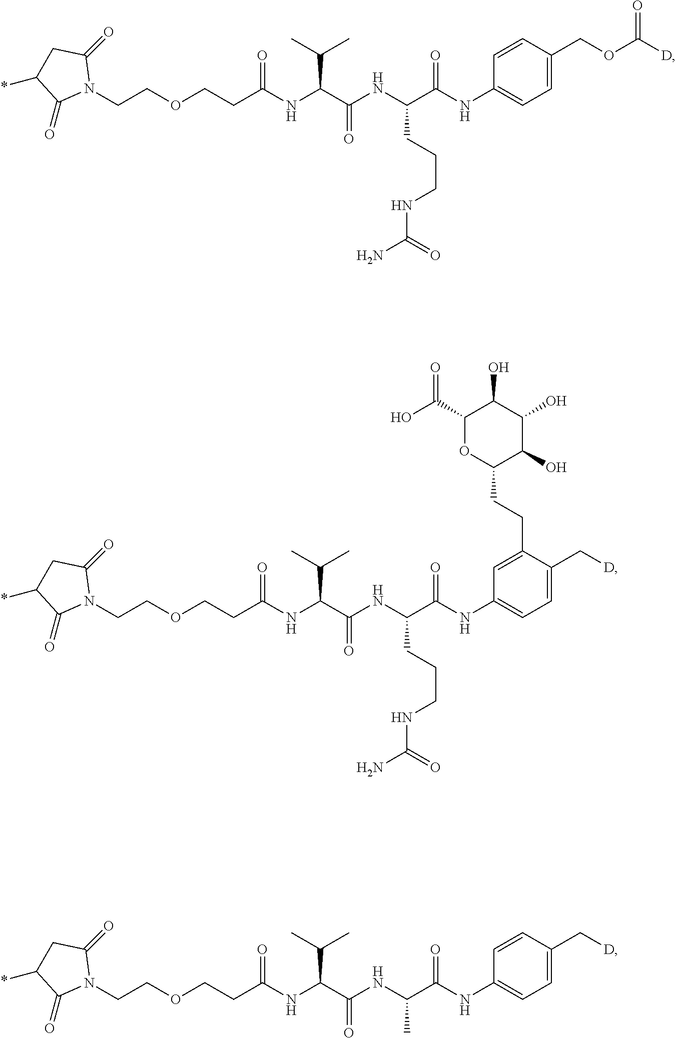

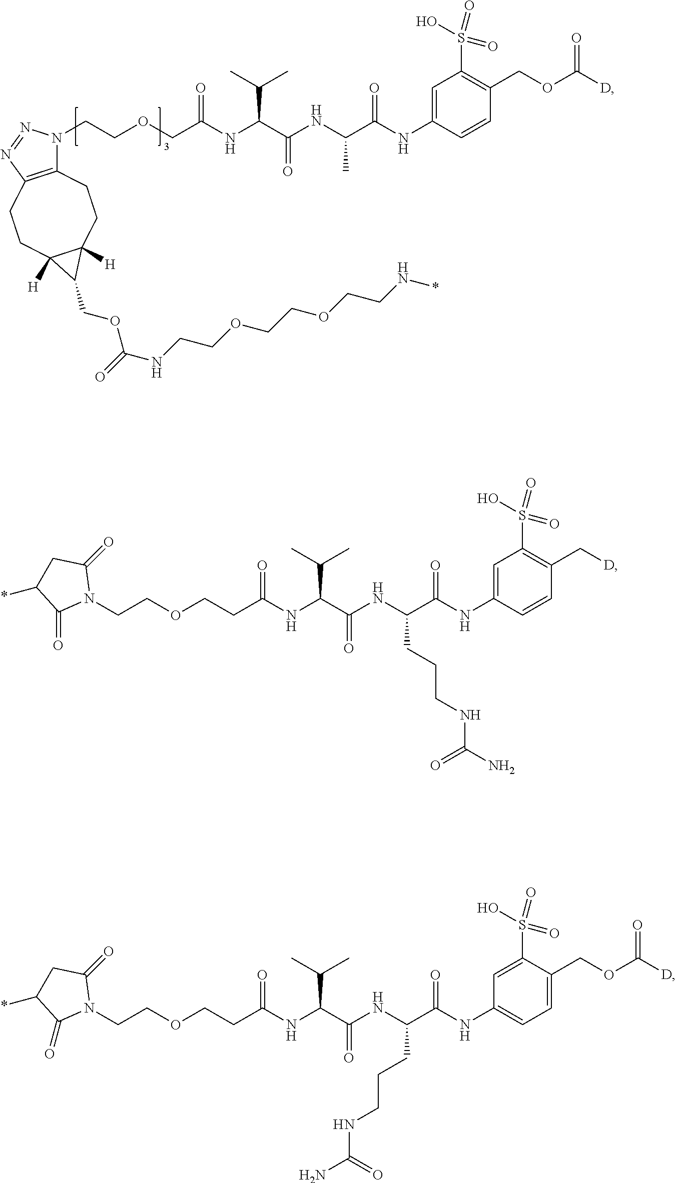

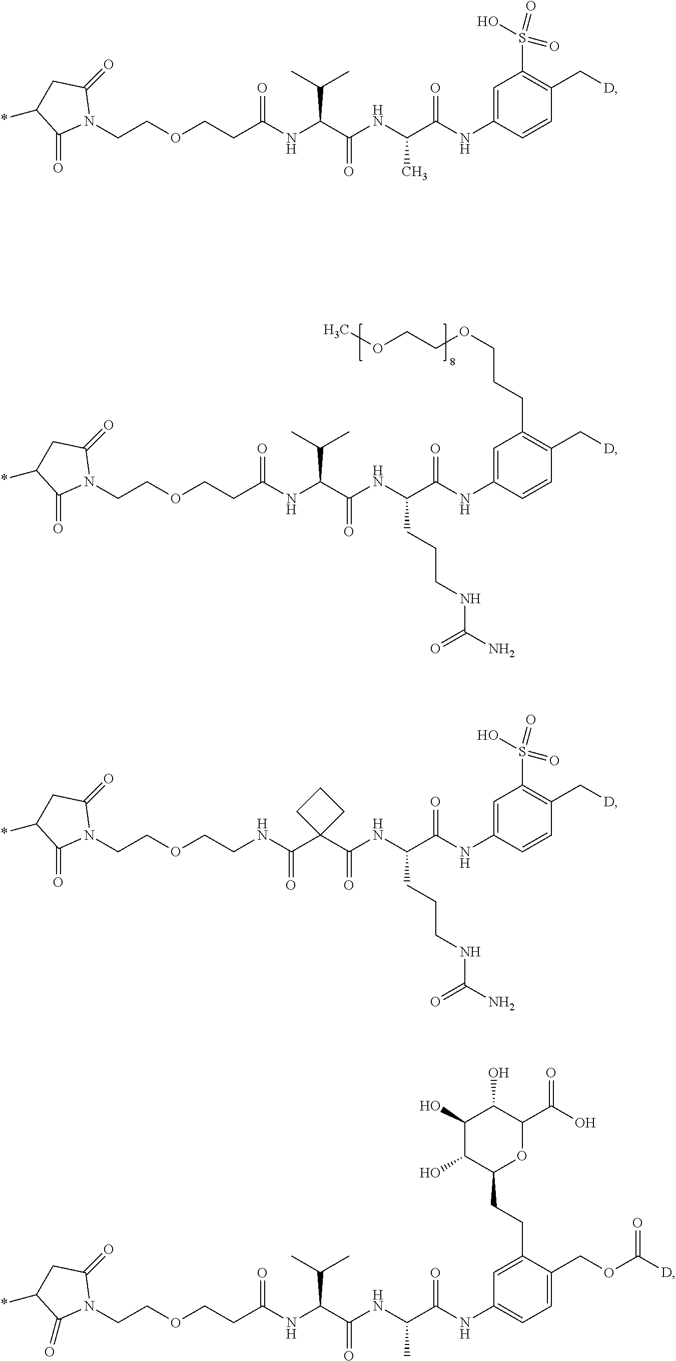

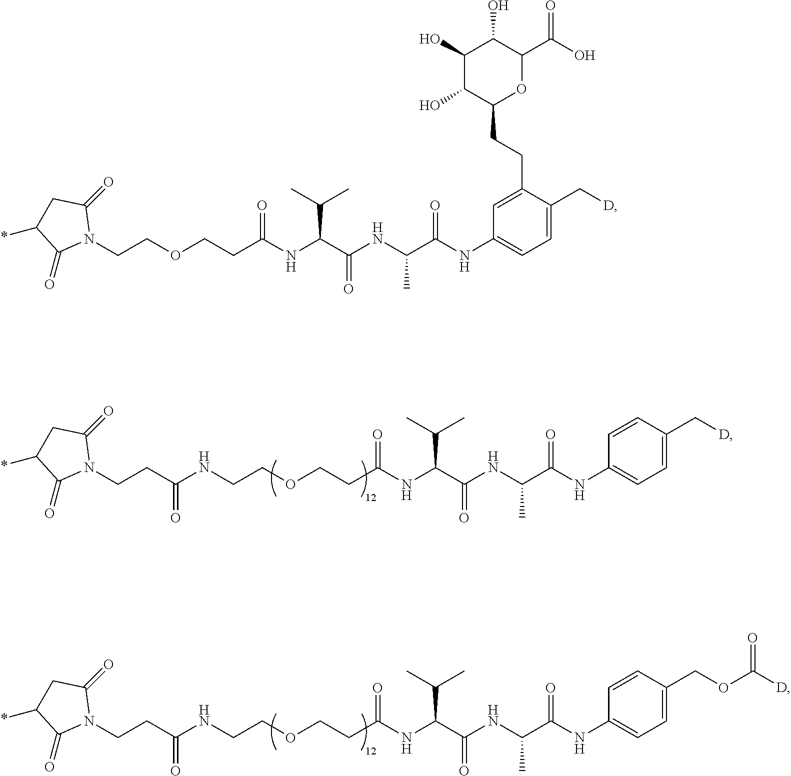

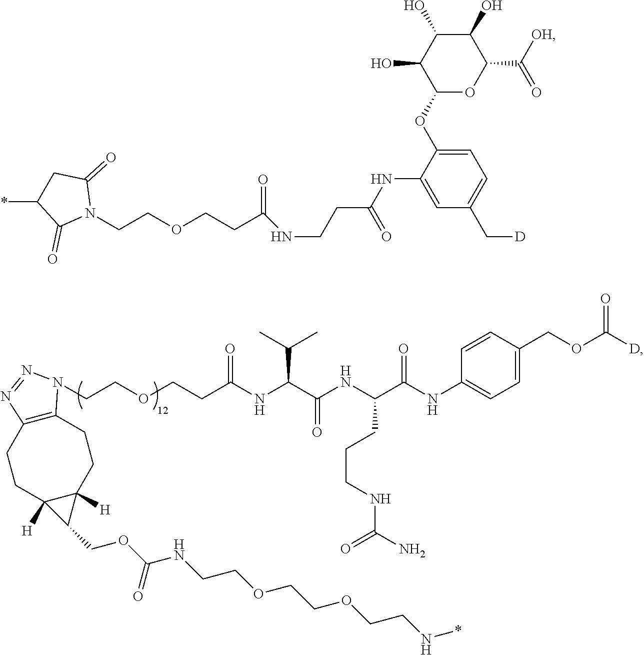

and
and wherein —* is a bond to the antibody or antigen-binding fragment thereof.

In some embodiments, the antibody-drug conjugate comprises the linker-drug group, -(L-D), which is of the formula (C):

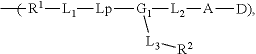

wherein: R¹ is an attachment group, L₁ is a bridging spacer; Lp is a peptide group comprising 1 to 6 amino acids; D is a panRAS inhibitor; G₁-L₂-A is a self-immolative spacer; L₂ is a bond, a methylene, a neopentylene or a C₂-C₃ alkenylene; A is a bond, —OC(═O)—*,

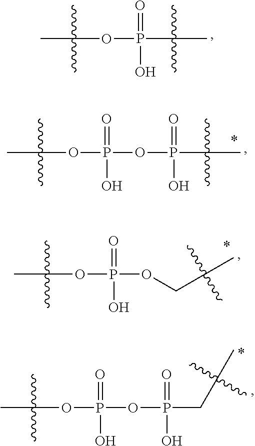 [SMILES: *OP(*)(=O)O ; *OP(=O)(O)OP(*)(=O)O ; *COP(=O)(O)O* ; *CP(=O)(O)OP(=O)(O)O*]

OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; L₃ is a spacer moiety; and R² is a hydrophilic moiety.

In some embodiments, the antibody-drug conjugate comprises the linker-drug group, -(L-D), which is of the formula (D):

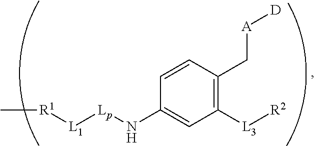

wherein: R¹ is an attachment group; Li is a bridging spacer; Lp is a peptide group comprising 1 to 6 amino acids; A is a bond, —OC(═O)—*,

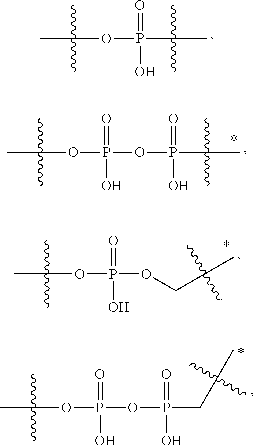 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; L₃ is a spacer moiety; and R² is a hydrophilic moiety.

In some embodiments, L₁ comprises:

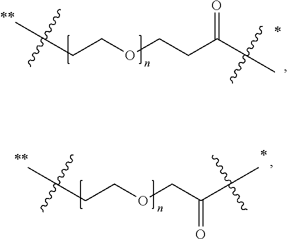

or *—CH(OH)CH(OH)CH(OH)CH(OH)—**, wherein each n is an integer from 1 to 12, wherein the * of L₁ indicates the point of direct or indirect attachment to Lp, and the ** of L₁ indicates the point of direct or indirect attachment to R¹.

In some embodiments, L₁ is

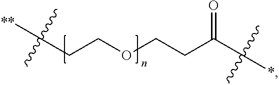

and n is an integer from 1 to 12 wherein the * of L₁ indicates the point of direct or indirect attachment to Lp, and the ** of L₁ indicates the point of direct or indirect attachment to R¹.

In some embodiments, L₁ is

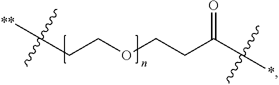

and n is 1, wherein the * of L₁ indicates the point of direct or indirect attachment to Lp, and the ** of L₁ indicates the point of direct or indirect attachment to R¹.

In some embodiments, L₁ is

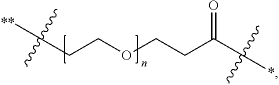

and n is 12, wherein the * of L₁ indicates the point of direct or indirect attachment to Lp, and the ** of L₁ indicates the point of direct or indirect attachment to R¹.

In some embodiments, L₁ is

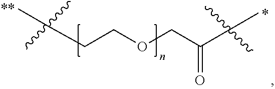

and n is an integer from 1 to 12, wherein the * of L₁ indicates the point of direct or indirect attachment to Lp, and the ** of L₁ indicates the point of direct or indirect attachment to R¹.

In some embodiments, L₁ comprises

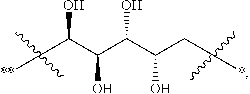

wherein the * of L₁ indicates the point of direct or indirect attachment to Lp, and the ** of L₁ indicates the point of direct or indirect attachment to R¹.

In some embodiments, L₁ is a bridging spacer comprising: *—C(═O)(CH₂)mO(CH₂)ₘ—**; *—C(═O)((CH₂)ₘO)ₜ(CH₂)ₙ—**; *—C(═O)(CH₂)ₘ—**; *—C(═O)N H((CH₂)ₘO)ₜ(CH₂)ₙ—**; *—C(═O)O(CH₂)ₘSSC(R³)₂(CH₂)ₘC(═O)NR³(CH₂)ₘNR³C(═O)(CH₂)ₘ—**; *—C(═O)O(CH₂)ₘC(═O)NH(CH₂)ₘ—**; *—C(═O)(CH₂)ₘNH(CH₂)ₘ—**; *—C(═O)(CH₂)ₘNH(CH₂)nC(═O)—**; *—C(═O)(CH₂)ₘX₁(CH₂)ₘ—**; *—C(═O)((CH₂)ₘO)ₜ(CH₂)ₙX₁(CH₂)ₙ—**; *—C(═O)(CH₂)ₘNHC(═O)(CH₂)ₙ—**; *—C(═O)((CH₂)ₘO)ₜ(CH₂)ₙNHC(═O)(CH₂)ₙ—**; *—C(═O)(CH₂)ₘNHC(═O)(CH₂)ₙX(CH₂)ₙ—**; *—C(═O)((CH₂)ₘO)ₜ(CH₂)ₙNHC(═O)(CH₂)ₙX(CH₂)ₙ—**; *—C(═O)((CH₂)ₘO)ₜ(CH₂)ₙC(═O)NH(CH₂)ₘ—**; *—C(═O)(CH₂)ₘC(R³)₂—** or *—C(═O)(CH₂)ₘC(═O)NH(CH₂)ₘ—**, where the * of L₁ indicates the point of direct or indirect attachment to Lp, and the ** of L₁ indicates the point of direct or indirect attachment to R¹, wherein X₁ is

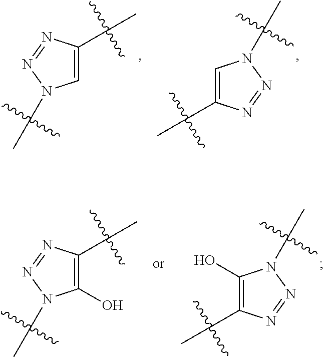 [SMILES: *c1cn(*)nn1 ; *c1cn(*)nn1 ; *c1nnn(*)c1O ; *c1nnn(*)c1O]

and
each m is independently selected from 1, 2, 3, 4, 5, 6, 7, 8, 9 and 10;
each n is independently selected from 1, 2, 3, 4, 5, 6, 7, 8, 9 and 10; and
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30.

In some embodiments, R² is a hydrophilic moiety comprising polyethylene glycol, polyalkylene glycol, a polyol, a polysarcosine, a sugar, an oligosaccharide, a polypeptide, C₂-C₆ alkyl substituted with 1 to 3

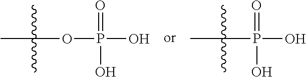 [SMILES: *OP(=O)(O)O ; *P(=O)(O)O]

groups, or C₂-C₆alkyl substituted with 1 to 2 substituents independently selected from —OC(═O)NHS(O)₂NHCH₂CH₂OCH₃, —NHC(═O)C₁₋₄alkylene-P(O)(OCH₂CH₃)₂ and —COOH groups. In some embodiments, R² is

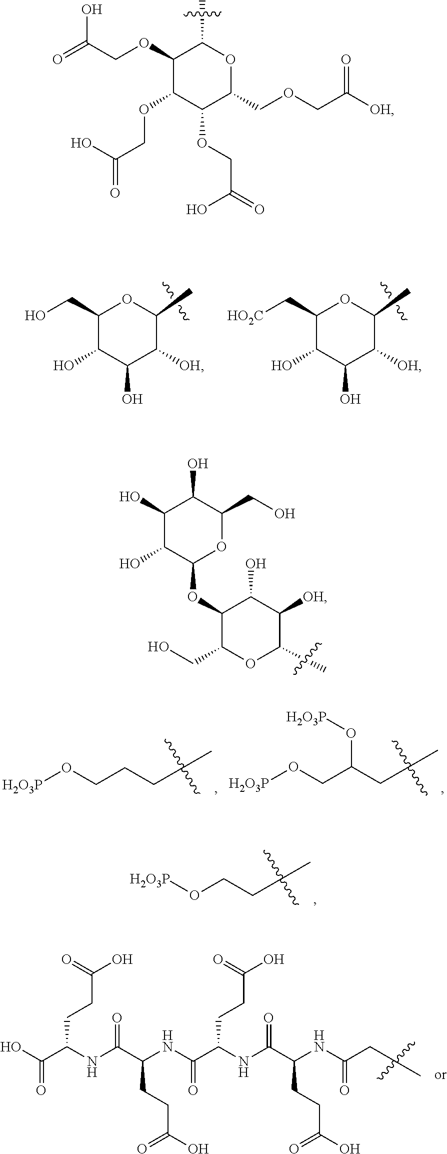

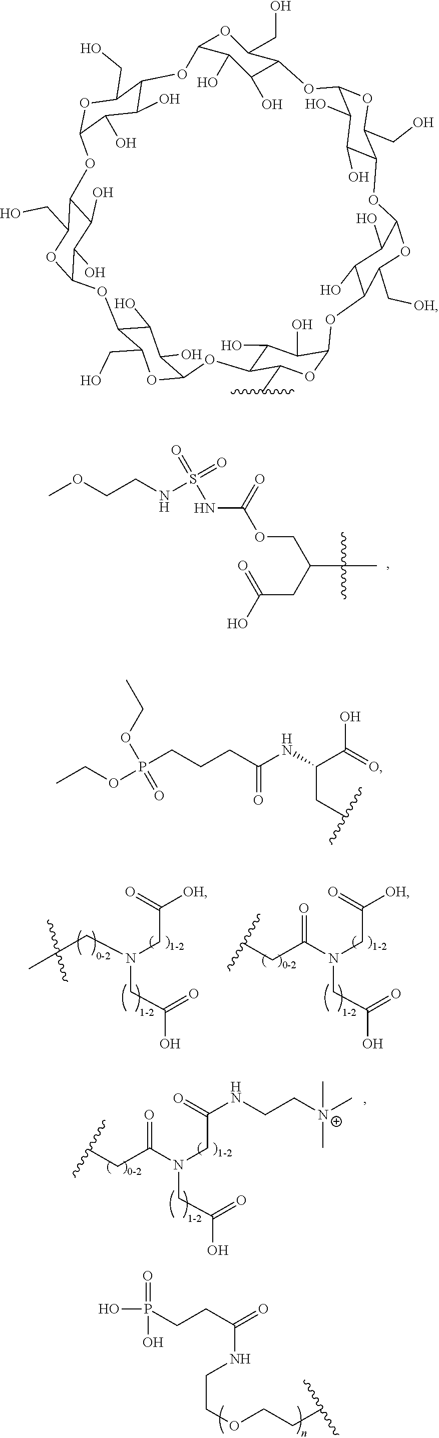

wherein n is an integer between 1 and 6,

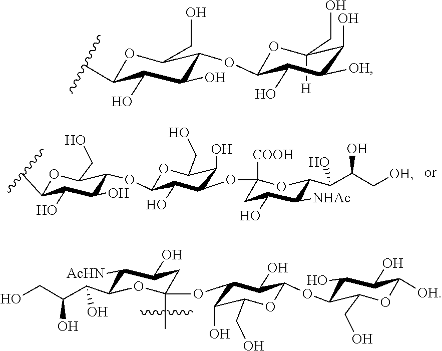

In some embodiments, the hydrophilic moiety comprises a polyethylene glycol of formula:

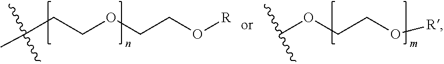

wherein R is H, —CHs CH_{Z}CH_{Z}NHC(═O)ORₐ, —CH₂CH₂NHC(═O)Rₐ, or —CH₂CH₂C(═O)ORₐ, R′ is OH, —OCH₃, CH₂CH₂NHC(═O)ORₐ, —CH₂CH₂NHC(═O)Rₐ, or —OCH₂CH₂C(═O)ORₐ, and each of m and n is an integer between 2 and 25 (e.g., between 3 and 25).

In some embodiments, the hydrophilic moiety comprises

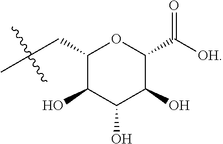 [SMILES: *C[C@@H]1O[C@H](C(=O)O)[C@@H](O)[C@H](O)[C@H]1O]

In some embodiments, the hydrophilic moiety comprises a polysarcosin, e.g., with the following moiety

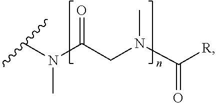

wherein n is an integer between 3 and 25; and R is H, —CH₃ or —CH₂CH₂C(═O)OH.

In some embodiments, L₃ is a spacer moiety having the structure

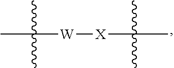

wherein:
W is —CH₂—, —CH₂O—, —CH₂N(R^{b})C(═O)O—, —NHC(═O)C(R^{b})₂NHC(═O)O—, —NHC(═O)C(R^{b})₂NH—, —NHC(═O)C(R^{b})₂NHC(═O)—, —CH₂N(X—R²)C(═O)O—, —C(═O)N(X—R²)—, —CH₂N(X—R²)C(═O)—, —C(═O)NR^{b}—, —C(═O)NH—, —CH₂NR^{b}C(═O)—, —CH₂NR^{b}C(═O)NH—, —CH₂NR^{b}C(═O)NR^{b}—, —NHC(═O)—, —NHC(═O)O—, —NHC(═O)NH—, —OC(═O)NH—, —S(O)₂NH—, —NHS(O)₂—, —C(═O)—, —C(═O)O— or —NH—, wherein each R^{b} is independently selected from H, C₁-C₆alkyl, and C₃-C₈ cycloalkyl; and
X is a bond, triazolyl or —CH₂-triazolyl-, wherein X is connected to R².

In some embodiments, L₃ is a spacer moiety having the structure

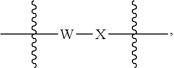

wherein:
W is —CH₂—, —CH₂O—, —CH₂N(R^{b})C(═O)O—, —NHC(═O)C(R^{b})₂NHC(═O)O—, —NHC(═O)C(R^{b})₂NH—, —NHC(═O)C(R^{b})₂NHC(═O)—, —CH₂N(X—R²)C(═O)O—, —C(═O)N(X—R²)—, —CH₂N(X—R²)C(═O)—, —C(═O)NR^{b}—, —C(═O)NH—, —CH₂NR^{b}C(═O)—, —CH₂NR^{b}C(═O)NH—, —CH₂NR^{b}C(═O)NR^{b}—, —NHC(═O)—, —NHC(═O)O—, —NHC(═O)NH—, —OC(═O)NH—, —S(O)₂NH—, —NHS(O)₂—, —C(═O)—, —C(═O)O— or —NH—, wherein each R^{b} is independently selected from H, C₁-C₆alkyl, and C₃-C₈ cycloalkyl; and
X is —CH₂-triazolyl-C₁₋₄ alkylene-OC(O)NHS(O)₂NH—, —C₄₋₆ cycloalkylene-OC(O)NHS(O)₂NH—, —(CH₂CH₂O)ₙ—C(O)NHS(O)₂NH—, —(CH₂CH₂O)ₙ—C(O)NHS(O)₂NH—(CH₂CH₂O)ₙ—, —CH₂-triazolyl-C₁₋₄ alkylene-OC(O)NHS(O)₂NH—(CH₂CH₂O)ₙ—, or —C₄₋₆ cycloalkylene-OC(O)NHS(O)₂NH—(CH₂CH₂O)ₙ—, wherein each n independently is 1, 2, or 3 and wherein X is connected to R².

In some embodiments, the attachment group is formed by a reaction comprising at least one reactive group. In some cases, the attachment group is formed by reacting: a first reactive group that is attached to the conjugate linker, and a second reactive group that is attached to the antibody or antigen-binding fragment thereof or is an amino acid residue of the antibody or antigen-binding fragment thereof.

In some embodiments, at least one of the reactive groups comprises:
a thiol,
a maleimide,
a haloacetamide,
an azide,
an alkyne,
a cyclcooctene,
a triaryl phosphine,
an oxanobornadiene,
a cyclooctyne,
a diaryl tetrazine,
a monoaryl tetrazine,
a norbornene,
an aldehyde,
a hydroxylamine,
a hydrazine,
NH₂—NH—C(═O)—,
a ketone,
a vinyl sulfone,
an aziridine,
an amino acid residue,

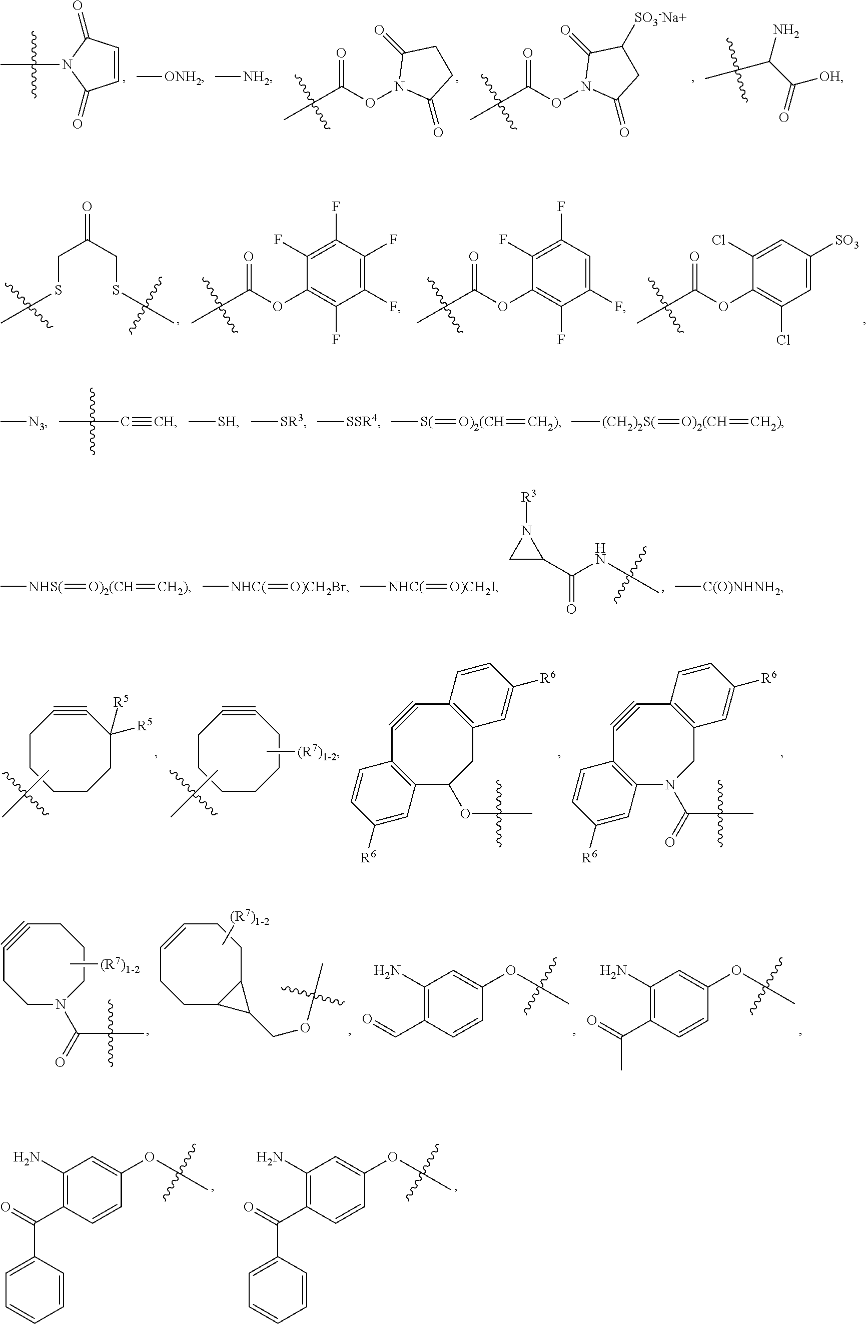

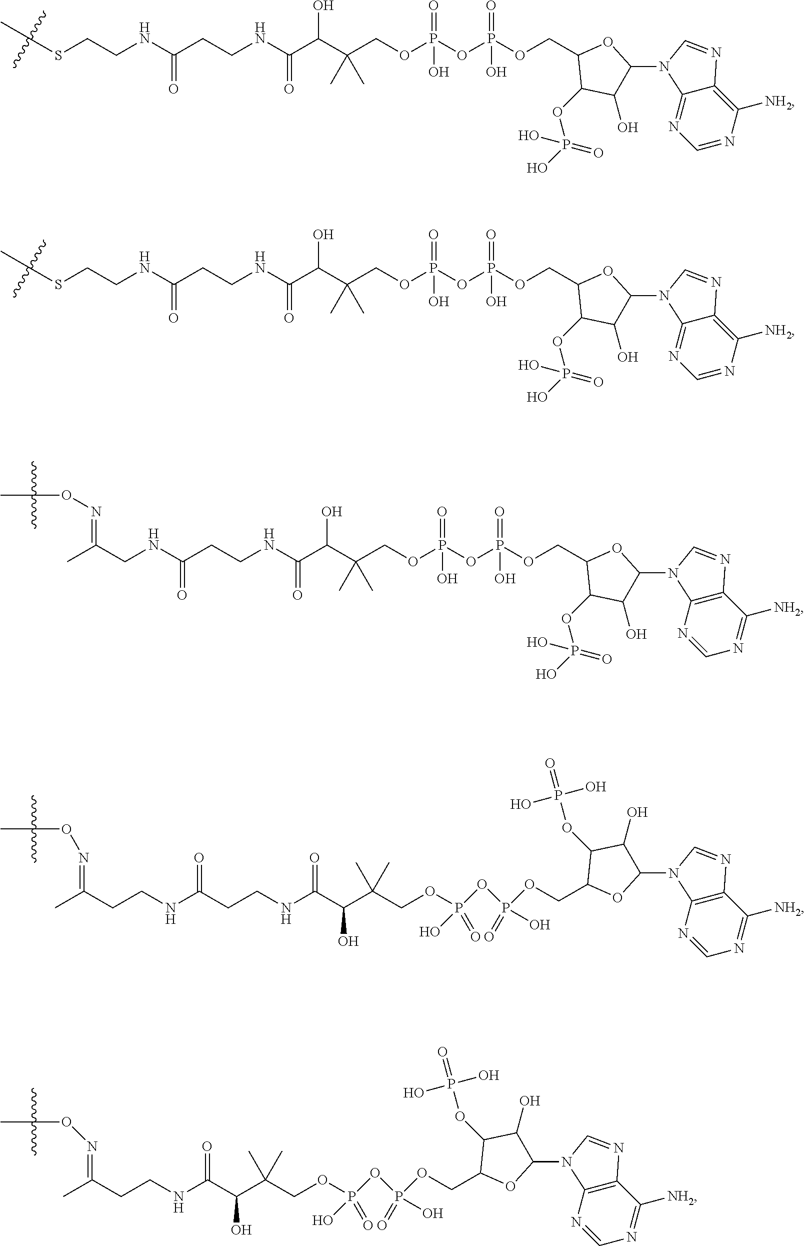

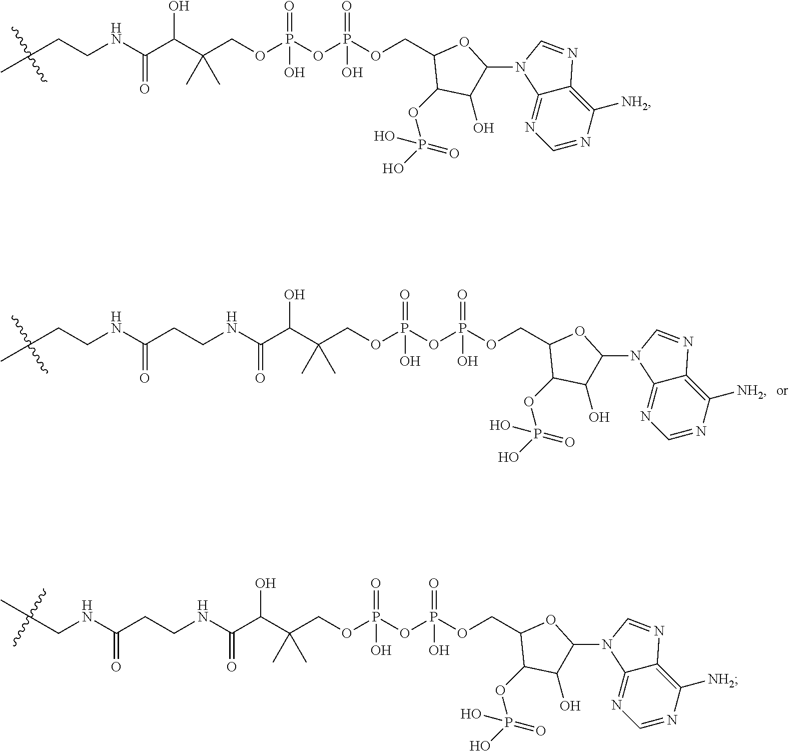

wherein:
each R³ is independently selected from H and C₁-C₆alkyl;
each R⁴ is 2-pyridyl or 4-pyridyl;
each R⁵ is independently selected from H, C₁-C₆alkyl, F, Cl, and —OH;
each R⁶ is independently selected from H, C₁-C₆alkyl, F, Cl, —NH₂, —OCH₃, —OCH₂CH₃, —N(CH₃)₂, —CN, —NO₂ and —OH;
each R⁷ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH.

In some embodiments, the first reactive group and second reactive group comprise:
a thiol and a maleimide,
a thiol and a haloacetamide,
a thiol and a vinyl sulfone,
a thiol and an aziridine,
an azide and an alkyne,
an azide and a cyclooctyne,
an azide and a cyclooctene,
an azide and a triaryl phosphine,
an azide and an oxanobornadiene,
a diaryl tetrazine and a cyclooctene,
a monoaryl tetrazine and a nonbornene,
an aldehyde and a hydroxylamine,
an aldehyde and a hydrazine,
an aldehyde and NH₂—NH—C(═O)—,
a ketone and a hydroxylamine,
a ketone and a hydrazine,
a ketone and NH2-NH—C(═O)—,
a hydroxylamine and

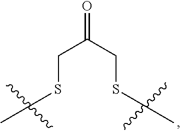 [SMILES: *SCC(=O)CS*]

an amine and

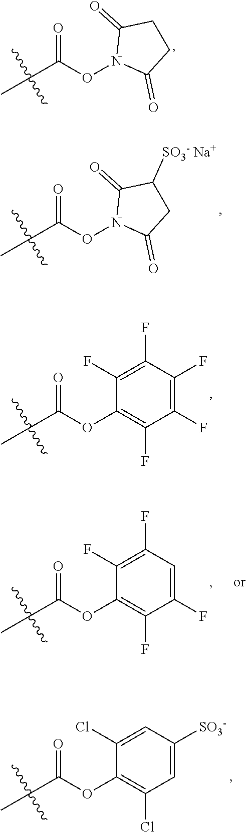

or
a CoA or CoA analogue and a serine residue.

In some embodiments, the attachment group comprises a group selected from:

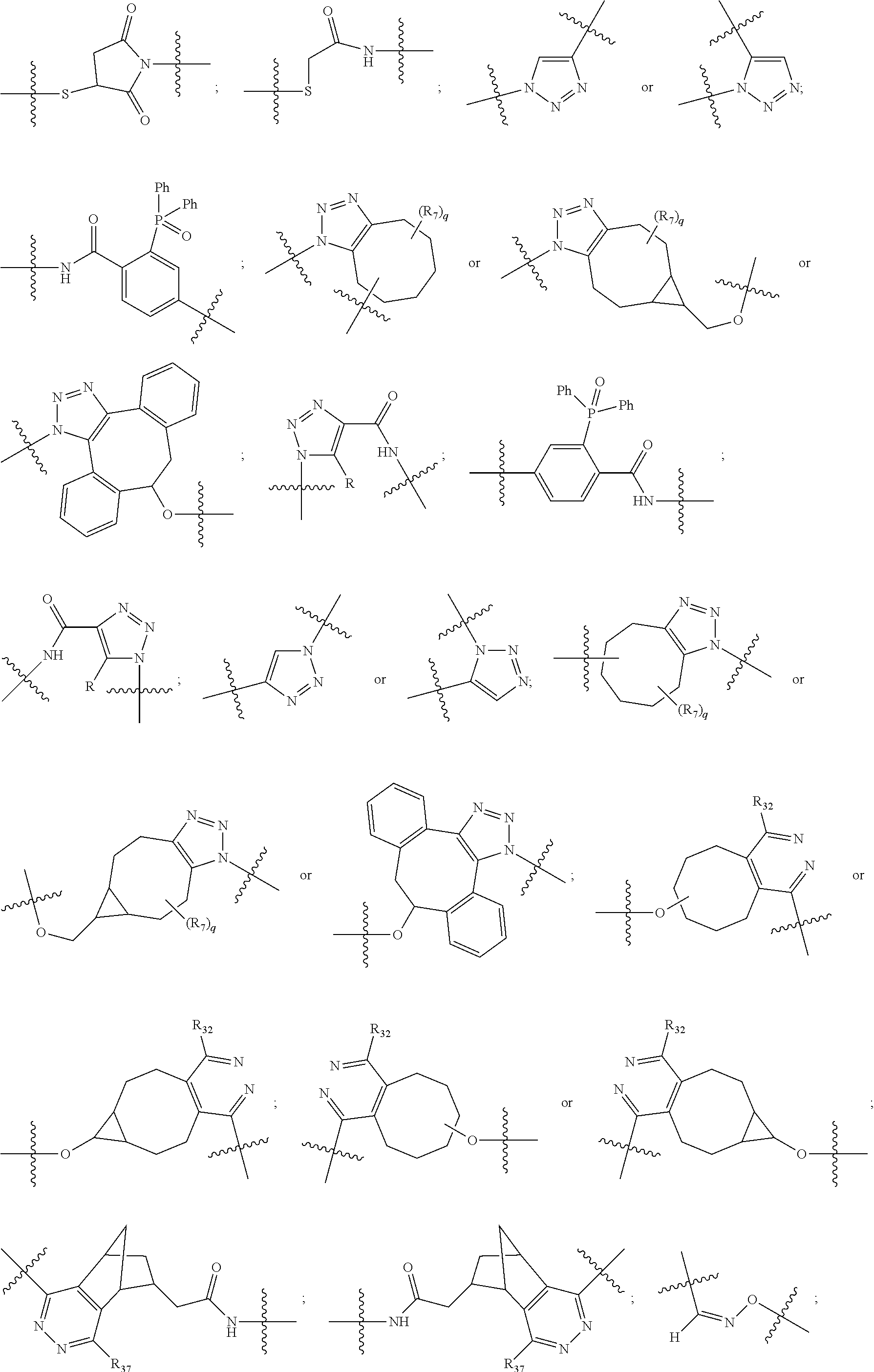

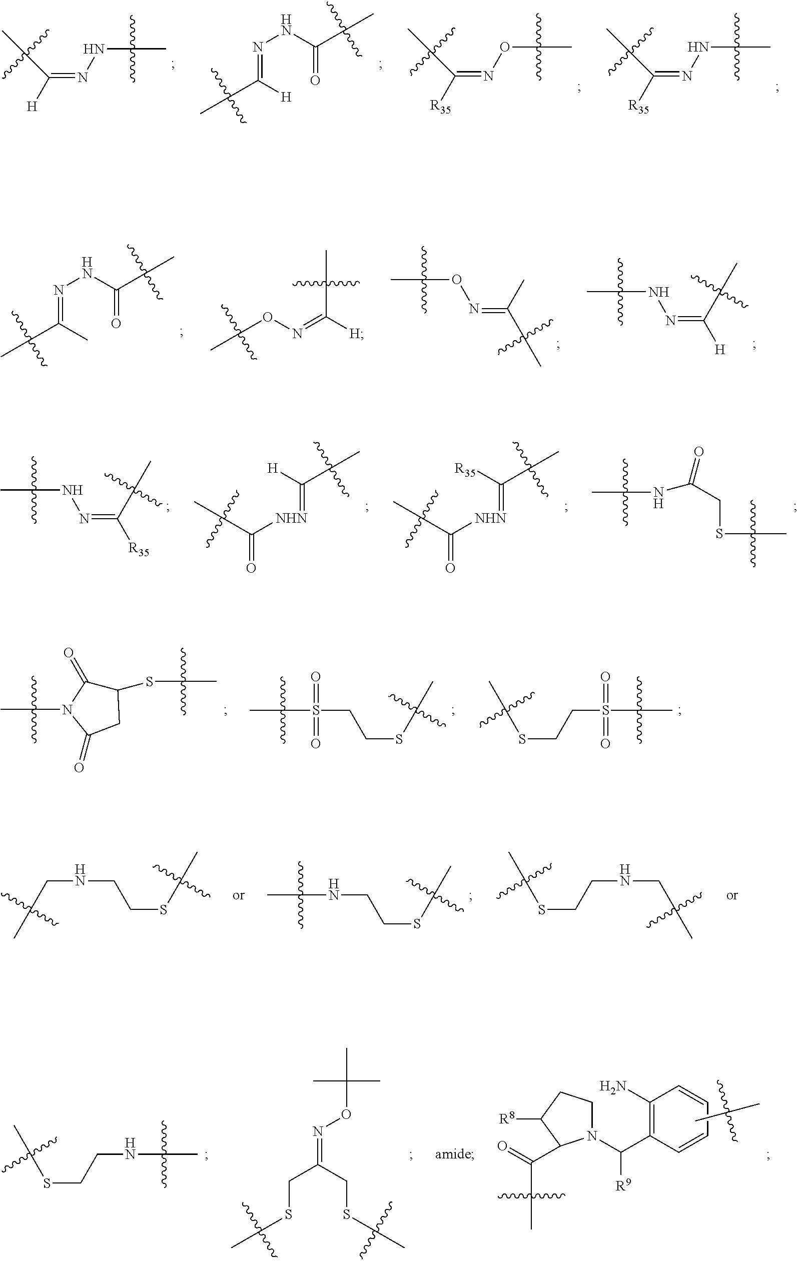

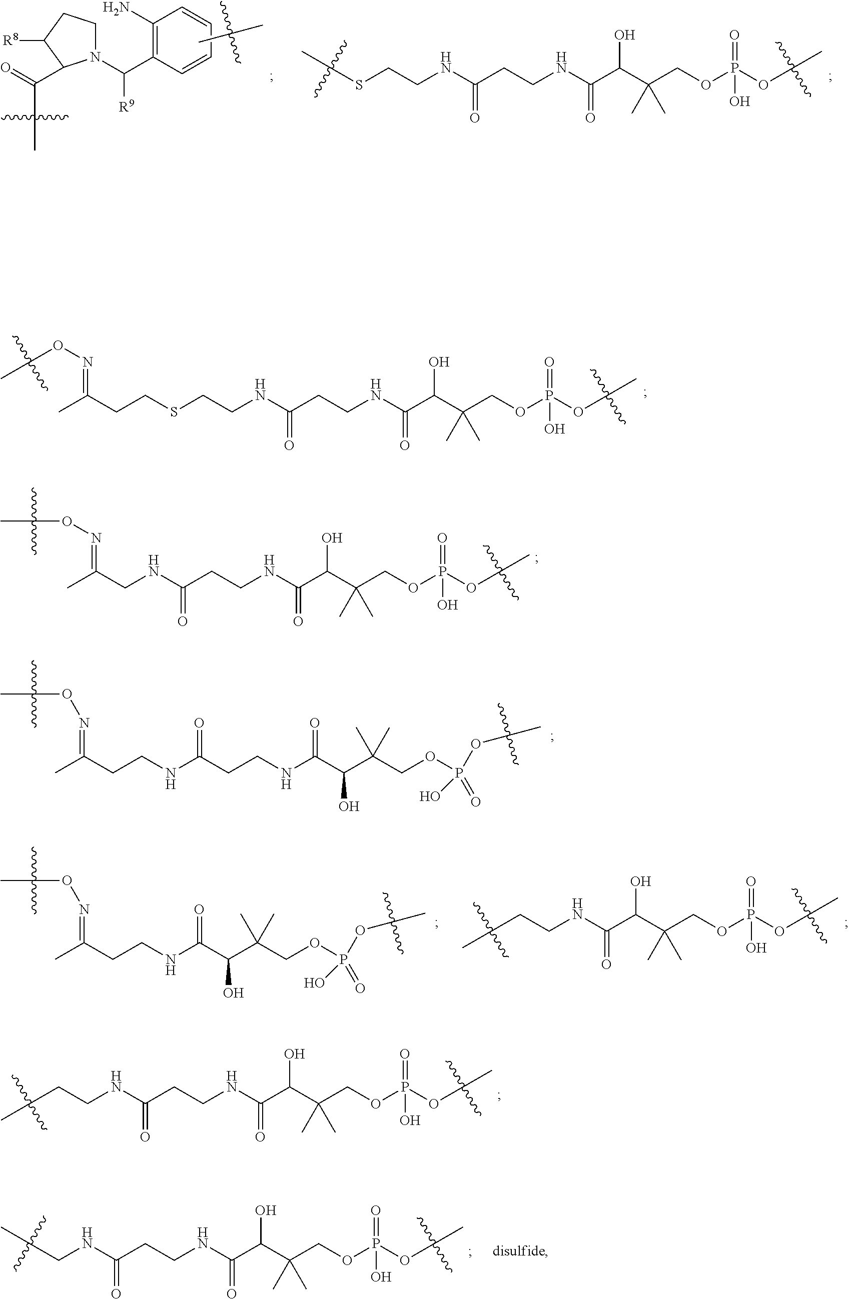

and
wherein:
R³² is H, C₁₋₄ alkyl, phenyl, pyrimidine or pyridine;
R³⁵ is H, C₁₋₆ alkyl, phenyl or C₁₋₄ alkyl substituted with 1 to 3 —OH groups;
each R⁷ is independently selected from H, C₁₋₆ alkyl, fluoro, benzyloxy substituted with —C(═O)OH, benzyl substituted with —C(═O)OH, C₁₋₄ alkoxy substituted with —C(═O)OH and C₁₋₄ alkyl substituted with —C(═O)OH;
R³⁷ is independently selected from H, phenyl and pyridine;
q is 0, 1, 2 or 3;
R⁸ is H or methyl; and
R⁹ is H, —CH₃ or phenyl.

In some embodiments, the peptide group (Lp) comprises 1 to 6 amino acid residues. In some embodiments, the peptide group (Lp) comprises 1 to 4 amino acid residues. In some embodiments, the peptide group comprises 1 to 3 amino acid residues. In some embodiments, the peptide group comprises 1 to 2 amino acid residues. In some embodiments, the amino acid residues are selected from L-glycine (Gly), L-valine (Val), L-citrulline (Cit), L-cysteic acid (sulfo-Ala), L-lysine (Lys), L-isoleucine (lie), L-phenylalanine (Phe), L-methionine (Met), L-asparagine (Asn), L-proline (Pro), L-alanine (Ala), L-leucine (Leu), L-tryptophan (Trp), and L-tyrosine (Tyr). In some embodiments, the peptide group comprises Val-Cit, Phe-Lys, Val-Ala, Val-Lys, Leu-Cit, sulfo-Ala-Val, and/or sulfo-Ala-Val-Ala. In some embodiments, Lp is selected from:

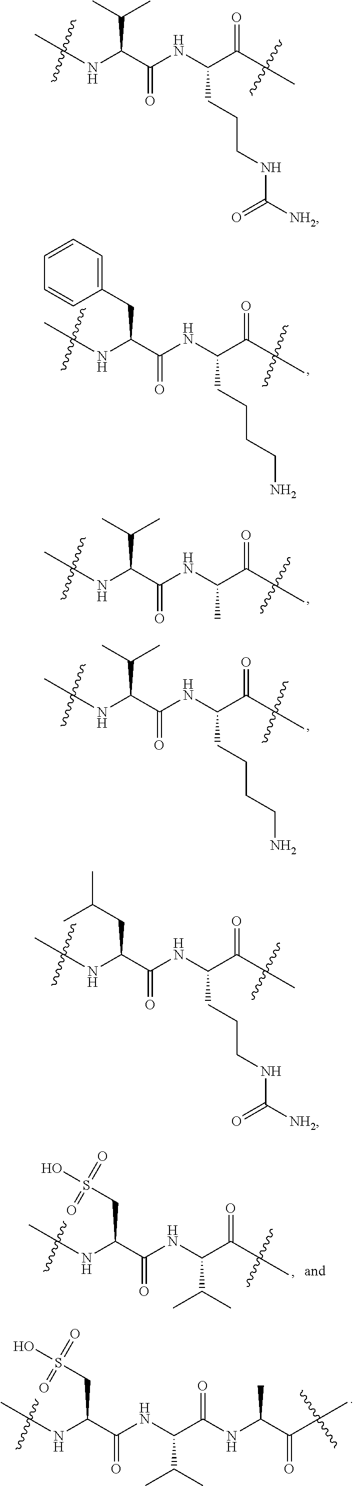

In some embodiments, the linker-drug group -(L-D) comprises or is formed from a compound of formula:

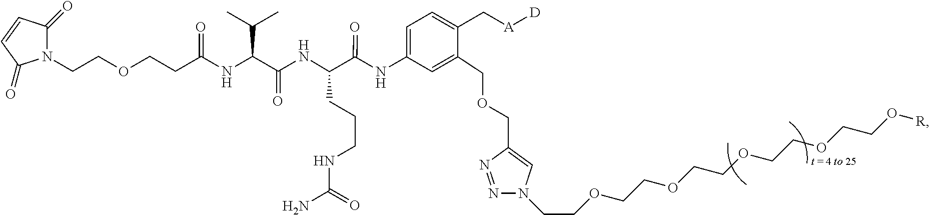

wherein:
R is H, —CH₃ or —CH₂CH₂C(═O)OH;
A is a bond, —OC(═O)—*,

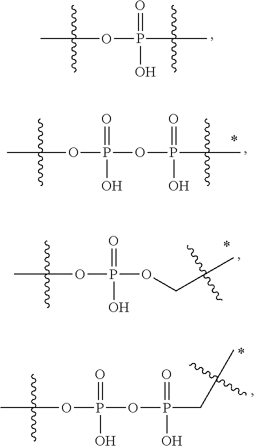 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a panRAS inhibitor. In some embodiments, the linker-drug group -(L-D) comprises the following formula:

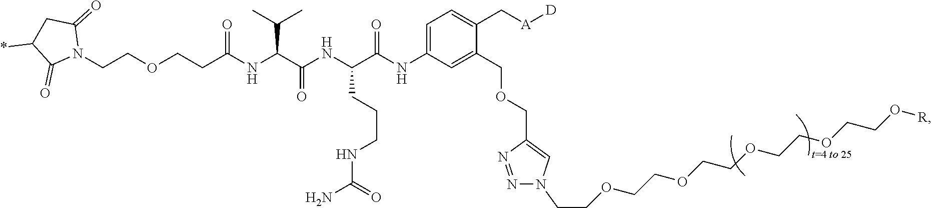

wherein:
—* is a bond to the antibody or antigen-binding fragment thereof; and A, D and R are as defined above. In some embodiments, A is a bond or —OC(═O)—*; and R is —CH₃ or —CH₂CH₂C(═O)OH.

In some embodiments, the linker-drug group -(L-D) comprises or is formed from a compound of formula:

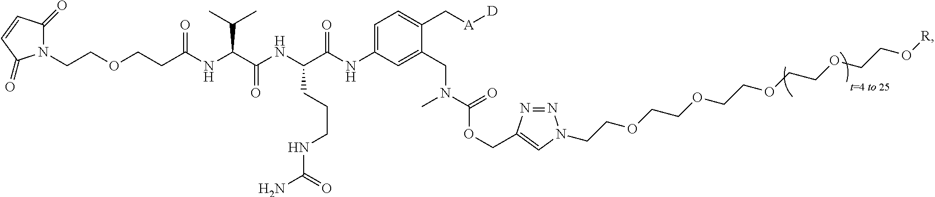

wherein:
R is H, —CH₃ or —CH₂CH₂C(═O)OH;
A is a bond, —OC(═O)—*,

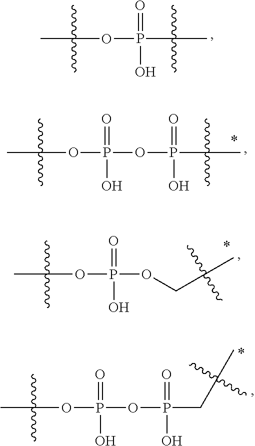 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a panRAS inhibitor. In some embodiments, the linker-drug group -(L-D) comprises the following formula:

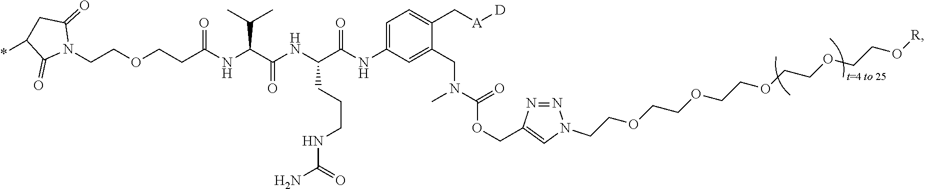

wherein: —* is a bond to the antibody or antigen-binding fragment thereof; and A, D and R are as defined above. In some embodiments, A is a bond or —OC(═O)—*; and R is —CH₃ or —CH₂CH₂C(═O)OH.

In some embodiments, the linker-drug group -(L-D) comprises or is formed from a compound of formula:

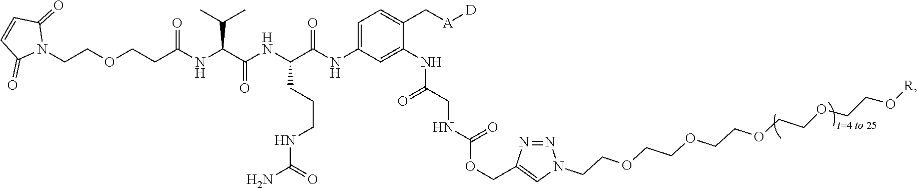

wherein:
R is H, —CH₃ or —CH₂CH₂C(═O)OH;
A is a bond, —OC(═O)—*,

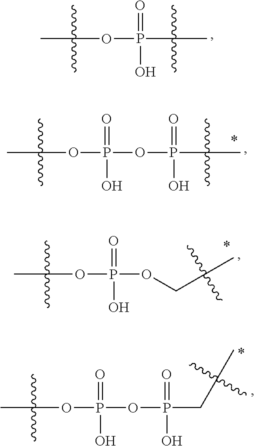 [SMILES: *OP(*)(=O)O ; *OP(=O)(O)OP(*)(=O)O ; *COP(=O)(O)O* ; *CP(=O)(O)OP(=O)(O)O*]

OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a panRAS inhibitor. In some embodiments, the linker-drug group -(L-D) comprises the following formula:

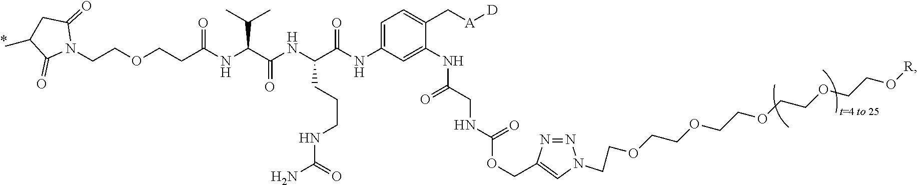

wherein:
_* is a bond to the antibody or antigen-binding fragment thereof; and A, D and R are as defined above. In some embodiments, A is a bond or —OC(═O)—*; and R is —CH₃ or —CH₂CH₂C(═O)OH.

In some embodiments, the linker-drug group -(L-D) comprises or is formed from a compound of formula:

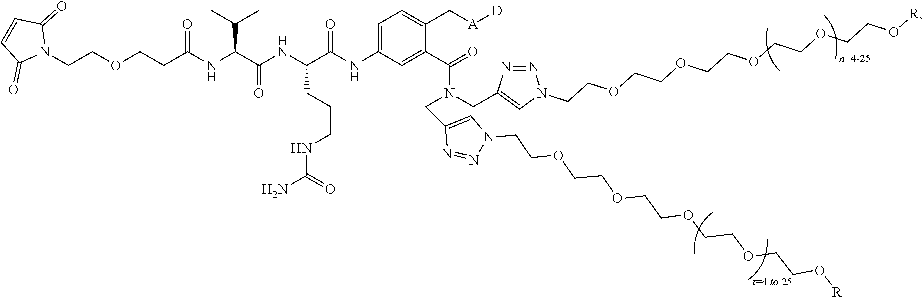

wherein:
each R is independently selected from H, —CH₃, and —CH₂CH₂C(═O)OH;
A is a bond, —OC(═O)—*,

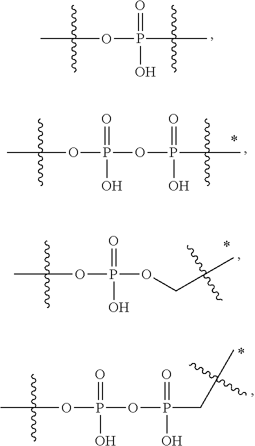 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆alkyl, and C₃-C₈ cycloalkyl and the * of A indicates the point of attachment to D; and
D is a panRAS inhibitor. In some embodiments, the linker-drug group -(L-D) comprises the following formula:

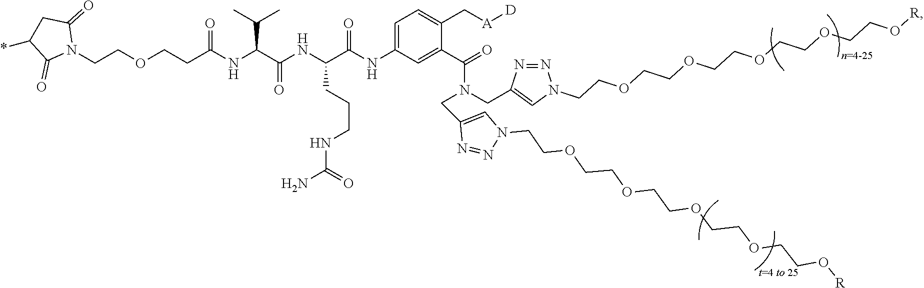

wherein:
—* is a bond to the antibody or antigen-binding fragment thereof; and A, D and R are as defined above. In some embodiments, A is a bond or —OC(═O)—*; and R is —CH₃ or —CH₂CH₂C(═O)OH.

In some embodiments, the linker-drug group -(L-D) comprises or is formed from a compound of formula:

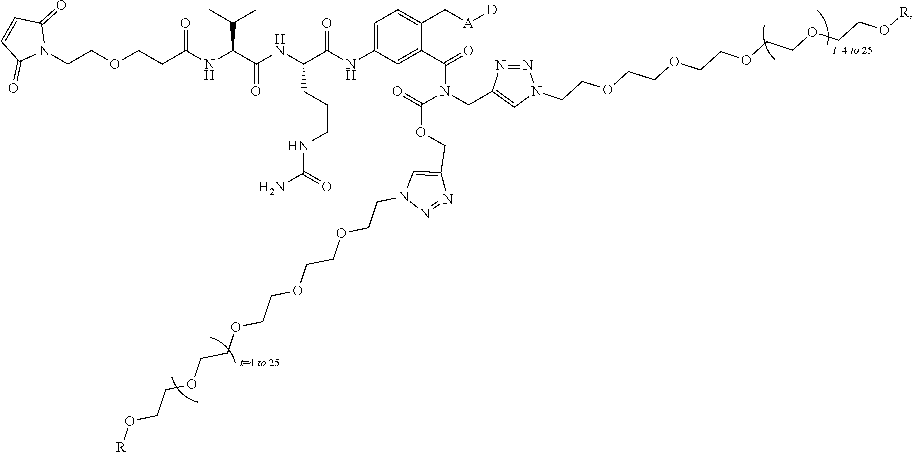

wherein:
each R is independently selected from H, —CH₃, and —CH₂CH₂C(═O)OH;
A is a bond, —OC(═O)—*,

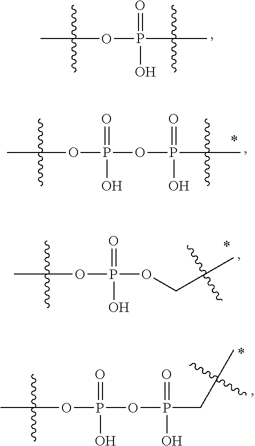 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a panRAS inhibitor. In some embodiments, the linker-drug group -(L-D) comprises the following formula:

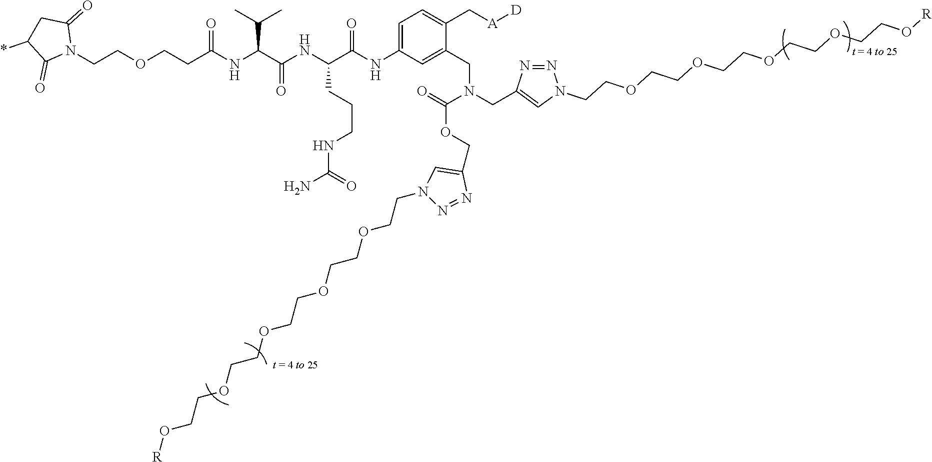

wherein: —* is a bond to the antibody or antigen-binding fragment thereof; and A, D and R are as defined above. In some embodiments, A is a bond or —OC(═O)—*; and R is —CH₃ or —CH₂CH₂C(═O)OH.

In some embodiments, the linker-drug group -(L-D) comprises or is formed from a compound of formula:

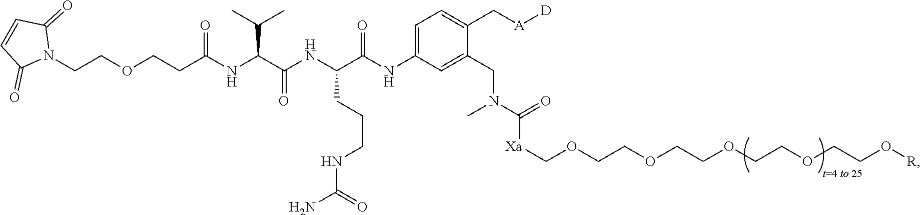

wherein:
Xa is —CH₂—, —OCH₂—, —NHCH₂— or —NRCH₂— and each R independently is H, —CH₃ or —CH₂CH₂C(═O)OH;
A is a bond, —OC(═O)—*,

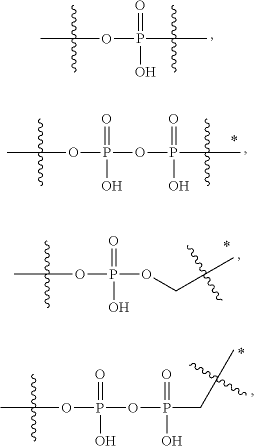 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a panRAS inhibitor. In some embodiments, the linker-drug group -(L-D) comprises the following formula:

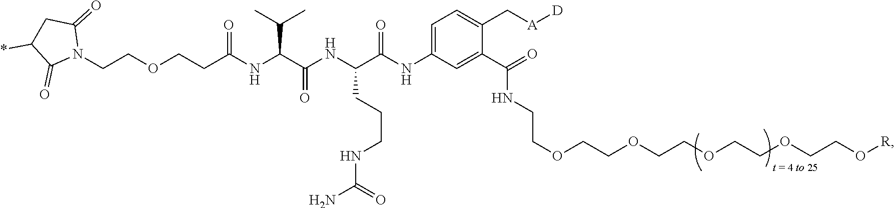

wherein: —* is a bond to the antibody or antigen-binding fragment thereof; and Xa, A, D and R are as defined above. In some embodiments, Xa is —CH₂— or —NHCH₂—; A is a bond or —OC(═O)—*; and R is —CH₃ or —CH₂CH₂C(═O)OH.

In some embodiments, the linker-drug group -(L-D) comprises or is formed from a compound of formula:

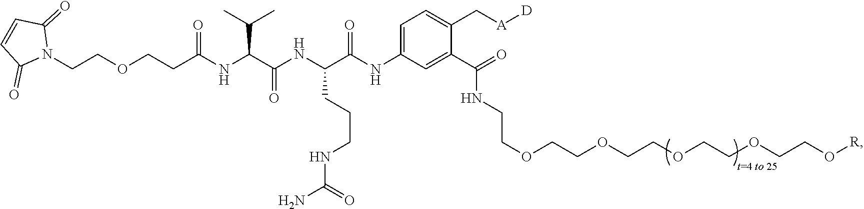

wherein:
R is H, —CH₃ or —CH₂CH₂C(═O)OH;
A is a bond, —OC(═O)—*,

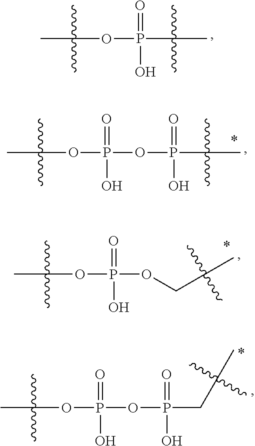 [SMILES: *OP(*)(=O)O ; *OP(=O)(O)OP(*)(=O)O ; *COP(=O)(O)O* ; *CP(=O)(O)OP(=O)(O)O*]

OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a panRAS inhibitor. In some embodiments, the linker-drug group -(L-D) comprises the following formula:

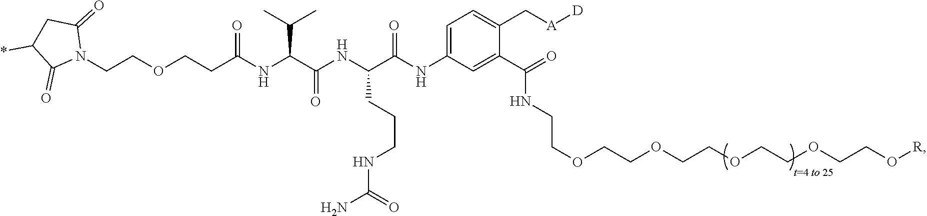

wherein: —* is a bond to the antibody or antigen-binding fragment thereof; and A, D and R are as defined above. In some embodiments, A is a bond or —OC(═O)—*; and R is —CH₃ or —CH₂CH₂C(═O)OH.

In some embodiments, the linker-drug group -(L-D) comprises or is formed from a compound of formula:

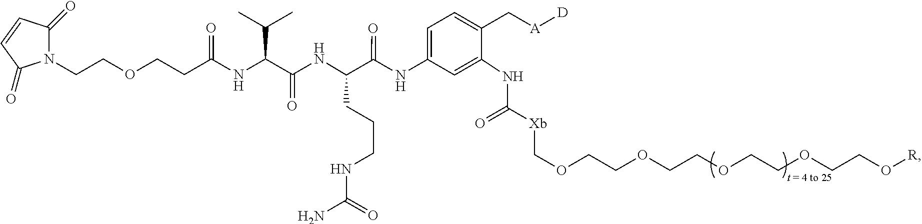

wherein:
Xb is —CH₂—, —OCH₂—, —NHCH₂— or —NRCH₂— and each R independently is H, —CH₃ or —CH₂CH₂C(═O)OH;
A is a bond, —OC(═O)—*,

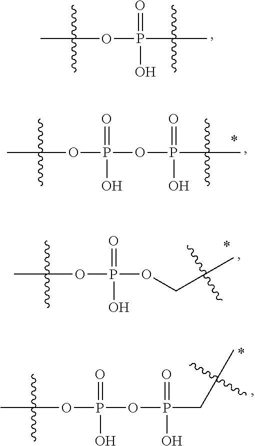 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a panRAS inhibitor. In some embodiments, the linker-drug group -(L-D) comprises the following formula:

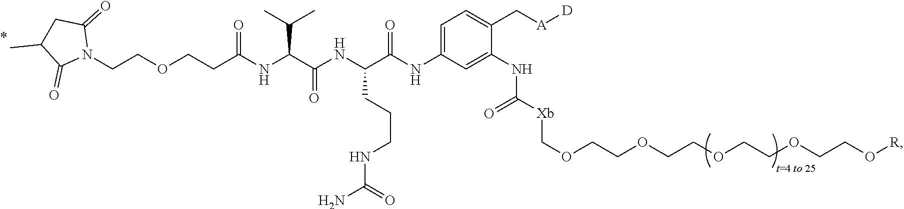

wherein —* is a bond to the antibody or antigen-binding fragment thereof; and Xb, A, D and R are as defined above. In some embodiments, A is a bond or —OC(═O)—*; and R is —CH₃ or —CH₂CH₂C(═O)OH.

In some embodiments, the linker-drug group -(L-D) comprises or is formed from a compound of formula:

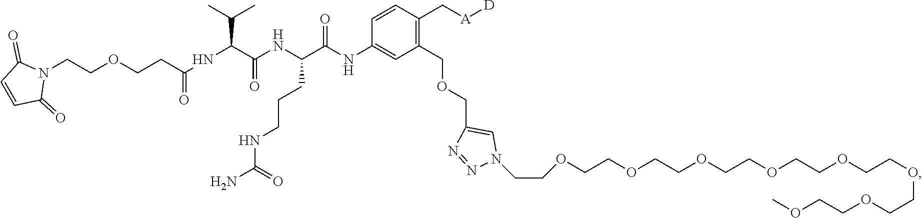

wherein:
A is a bond, —OC(═O)—*,

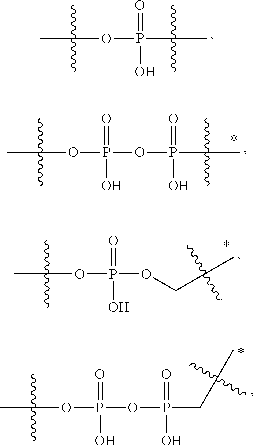 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a panRAS inhibitor. In some embodiments, the linker-drug group -(L-D) comprises the following formula:

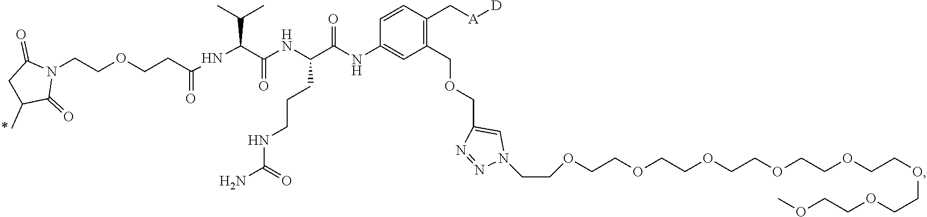

wherein: —* is a bond to the antibody or antigen-binding fragment thereof; and A and are as defined above. In some embodiments, A is a bond or —OC(═O)—*.

In some embodiments, the linker-drug group -(L-D) comprises or is formed from a compound of formula:

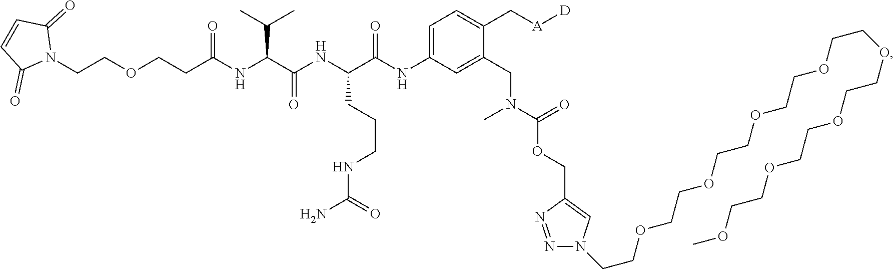

wherein:
A is a bond, —OC(═O)—*,

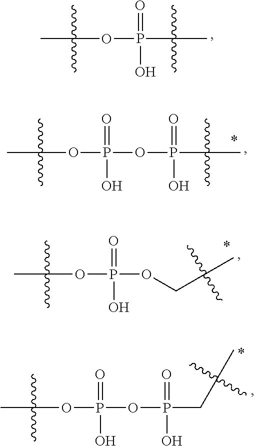 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a panRAS inhibitor. In some embodiments, the linker-drug group -(L-D) comprises the following formula:

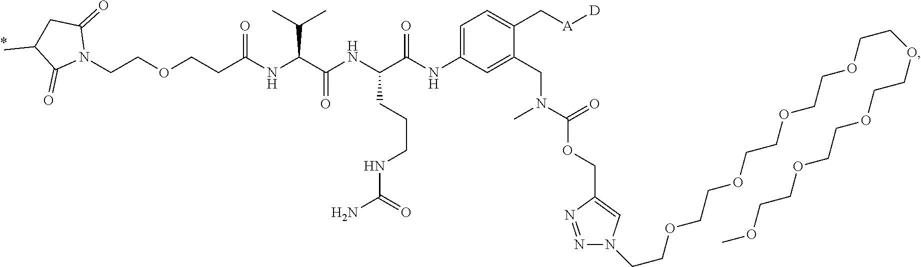

wherein: —* is a bond to the antibody or antigen-binding fragment thereof; and A and D are as defined above. In some embodiments, A is a bond or —OC(═O)—*.

In some embodiments, the linker-drug group -(L-D) comprises or is formed from a compound of formula:

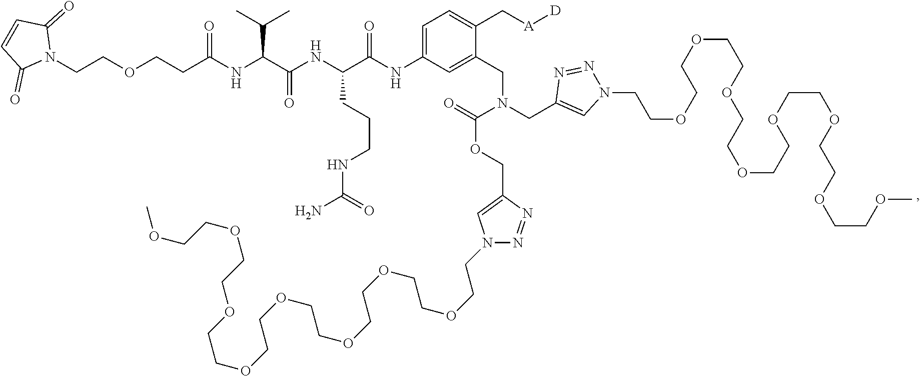

wherein:
A is a bond, —OC(═O—*,

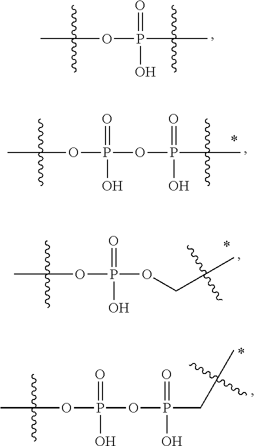 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a panRAS inhibitor. In some embodiments, the linker-drug group -(L-D) comprises the following formula:

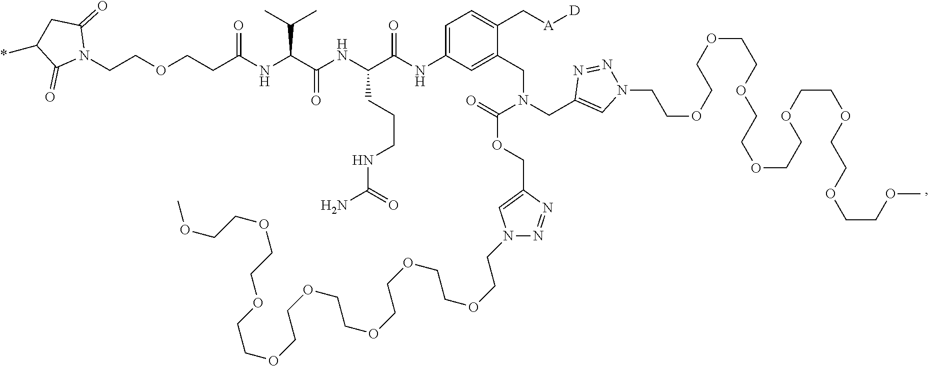

wherein: —* is a bond to the antibody or antigen-binding fragment thereof; and A and D are as defined above. In some embodiments, A is a bond or —OC(═O)—*.

In some embodiments, the linker-drug group -(L-D) comprises or is formed from a compound of formula:

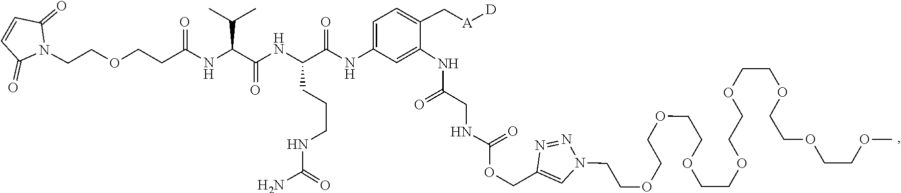

wherein:
A is a bond, —OC(═O)—*,

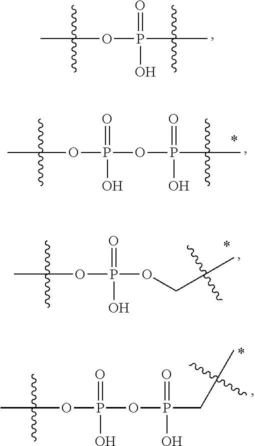 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a panRAS inhibitor. In some embodiments, the linker-drug group -(L-D) comprises the following formula:

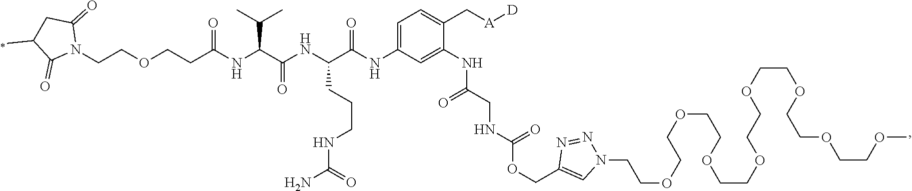

wherein: —* is a bond to the antibody or antigen-binding fragment thereof; and A and D are as defined above. In some embodiments, A is a bond or —OC(═O)—*.

In some embodiments, the linker-drug group -(L-D) comprises or is formed from a compound of formula:

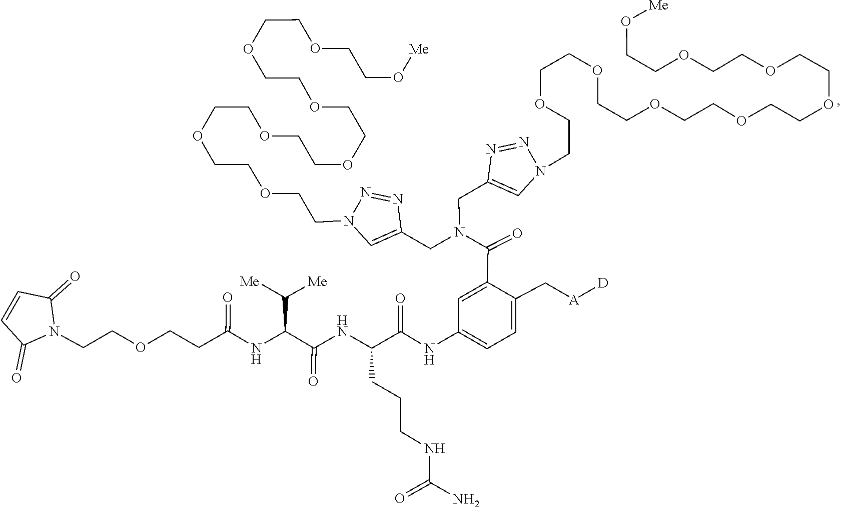

wherein:

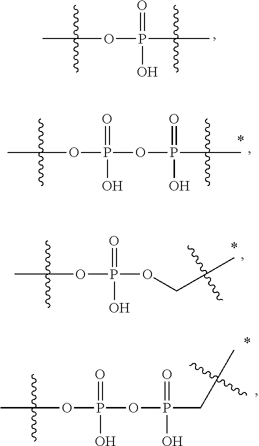 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or
—OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a panRAS inhibitor. In some embodiments, the linker-drug group -(L-D) comprises the following formula:

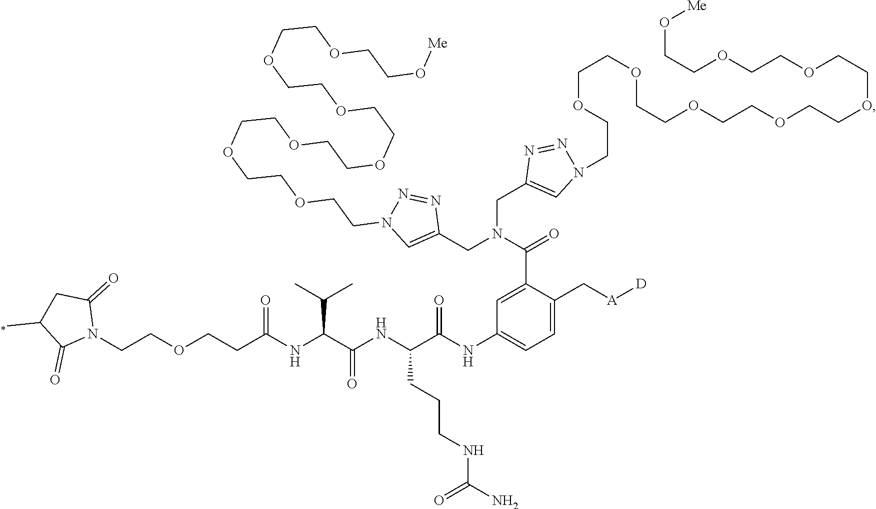

wherein: —* is a bond to the antibody or antigen-binding fragment thereof; and A and D are as defined above. In some embodiments, A is a bond or —OC(═O)—*.

In some embodiments, the linker-drug group -(L-D) comprises or is formed from a compound of formula:

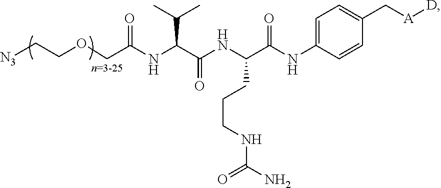

wherein:
A is a bond, —OC(═)*,

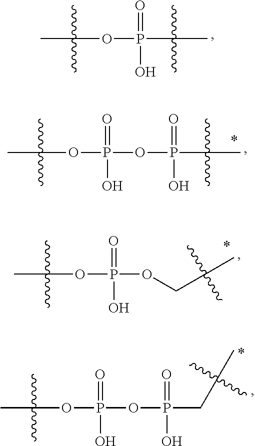 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a panRAS inhibitor. In some embodiments, the linker-drug group -(L-D) comprises the following formula:

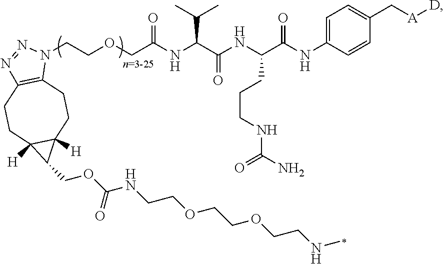

wherein: —* is a bond to the antibody or antigen-binding fragment thereof; and A and D are as defined above. In some embodiments, A is a bond or —OC(═O)—*.

In some embodiments, the linker-drug group -(L-D) comprises or is formed from a compound of formula:

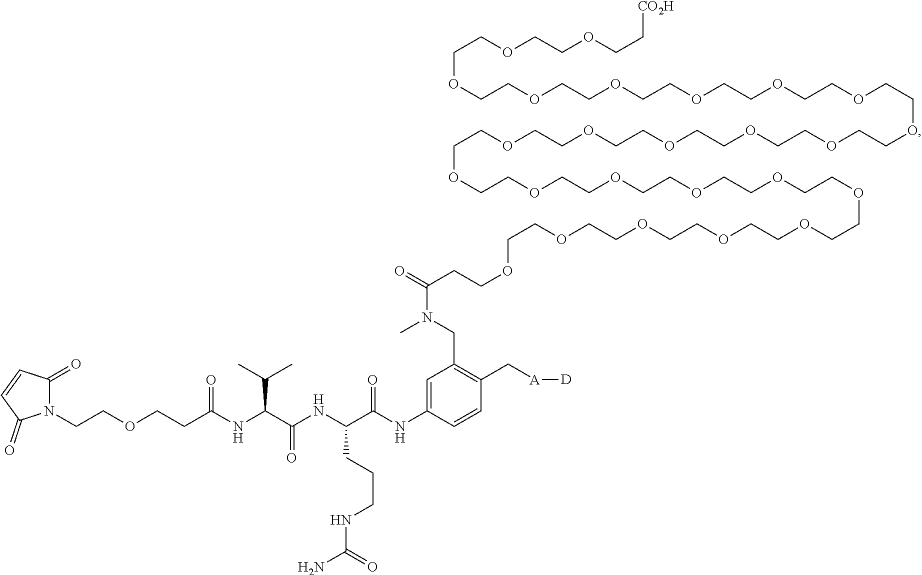

wherein:
A is a bond, —OC(═O)—*,

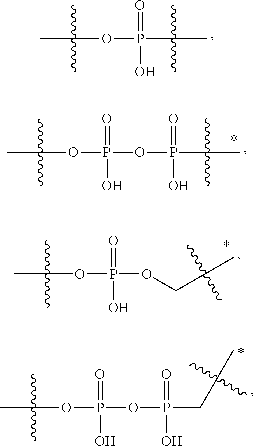 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a panRAS inhibitor. In some embodiments, the linker-drug group -(L-D) comprises the following formula:

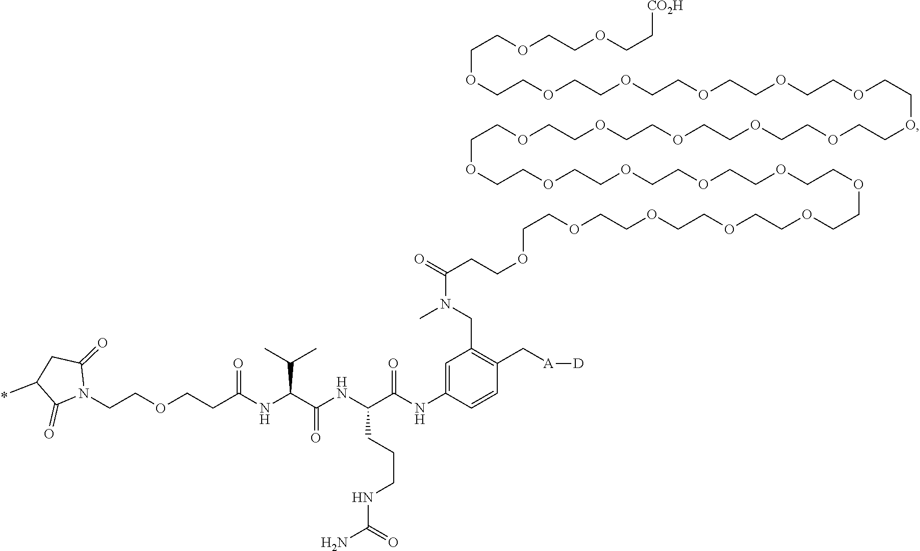

wherein: —* is a bond to the antibody or antigen-binding fragment thereof; and A and D are as defined above. In some embodiments, A is a bond or —OC(═O)—*.

In some embodiments, the linker-drug group -(L-D) comprises or is formed from a compound of formula:

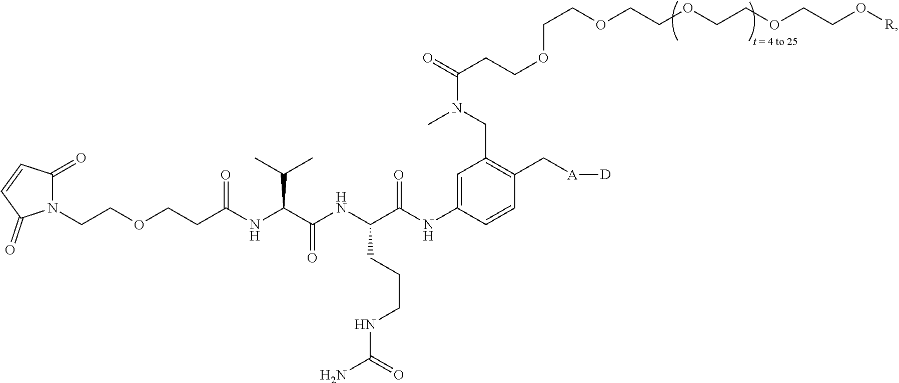

wherein:
each R independently is H, —CH₃ or —CH₂CH₂C(═O)OH;
A is a bond, OC(═OC)—*,

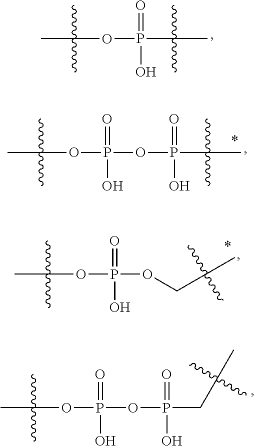 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*,
wherein each Rd is independently selected from H, C₁-C₆ alkyl, and C₃-C₈ cycloalkyl and the * of A indicates the point of attachment to 0; and
D is a panRAS inhibitor. In some embodiments, the linker-drug group -(L-D) comprises the following formula:

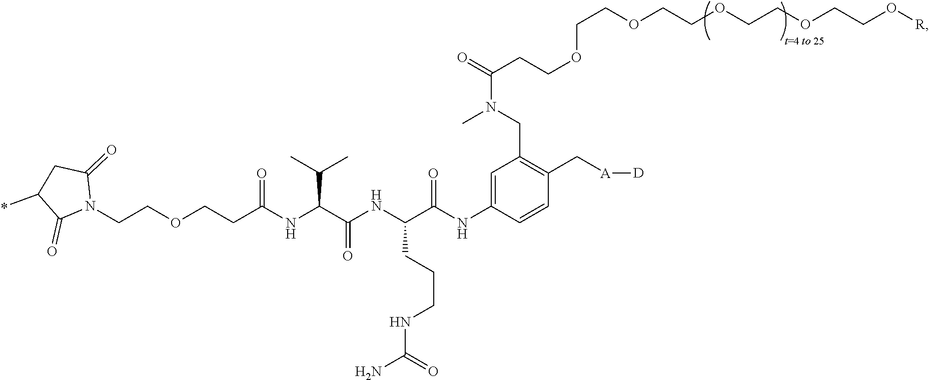

wherein: —* is a bond to the antibody or antigen-binding fragment thereof; and A, D and R are as defined above. In some embodiments, A is a bond or —OC(═O)—*; and R is —CH₃ or —CH₂CH₂C(═O)OH.

In some embodiments, the linker-drug group -(L-D) comprises or is formed from a compound of formula:

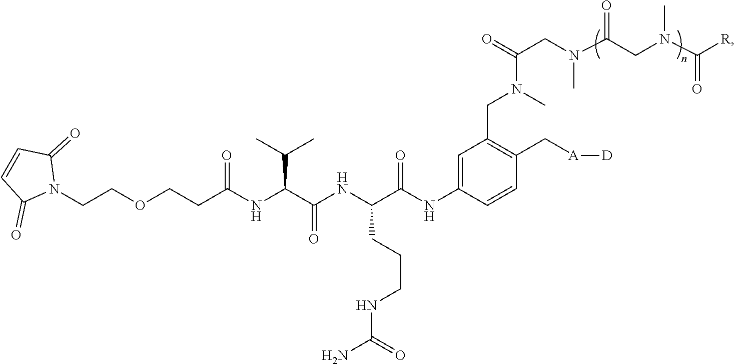

wherein:
each R independently is H, —CH₃ or —CH₂CH₂C(═O)OH;
A is a bond, —OC(═O)—*,

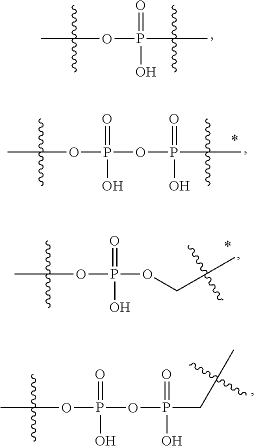 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a panRAS inhibitor. In some embodiments, the linker-drug group -(L-D) comprises the following formula:

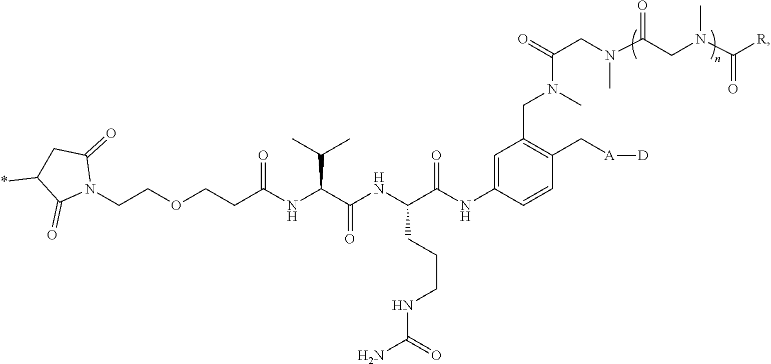

wherein: —* is a bond to the antibody or antigen-binding fragment thereof; and A, D and R are as defined above. In some embodiments, A is a bond or —OC(═O)—*; and R is —CH₃ or —CH₂CH₂C(═O)OH.

In some embodiments, the linker-drug group -(L-D) comprises or is formed from a compound of formula:

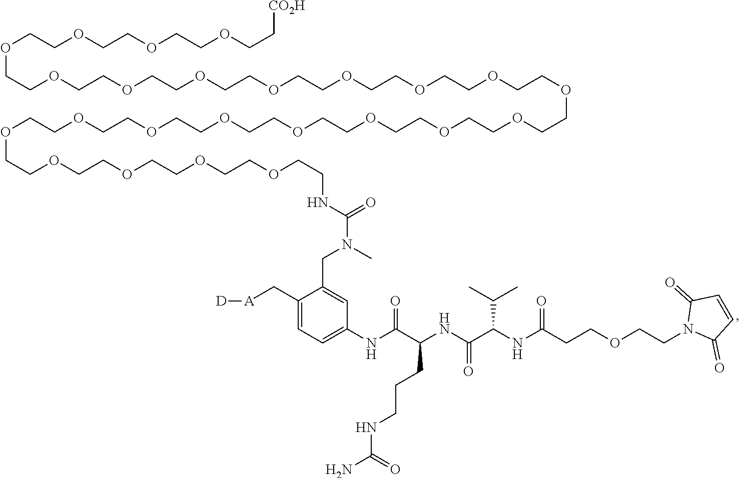

wherein:
A is a bond, —OC(═O)—*,

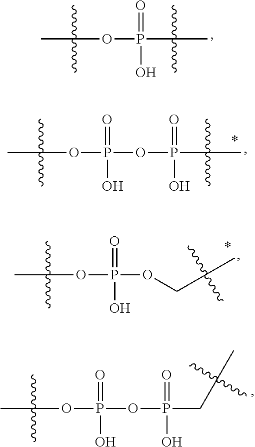 [SMILES: *OP(*)(=O)O ; *OP(=O)(O)OP(*)(=O)O ; *COP(=O)(O)O* ; *CP(=O)(O)OP(=O)(O)O*]

13 OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a panRAS inhibitor.

In some embodiments, A is a bond.

In some embodiments, A is —OC(═O)—*.

In some embodiments, R is —CH₃.

In some embodiments, R is —CH₂CH₂OOOH.

In some embodiments, the antibody-drug conjugate comprises the linker-drug group, -(L-D), which is formed from a compound selected from:

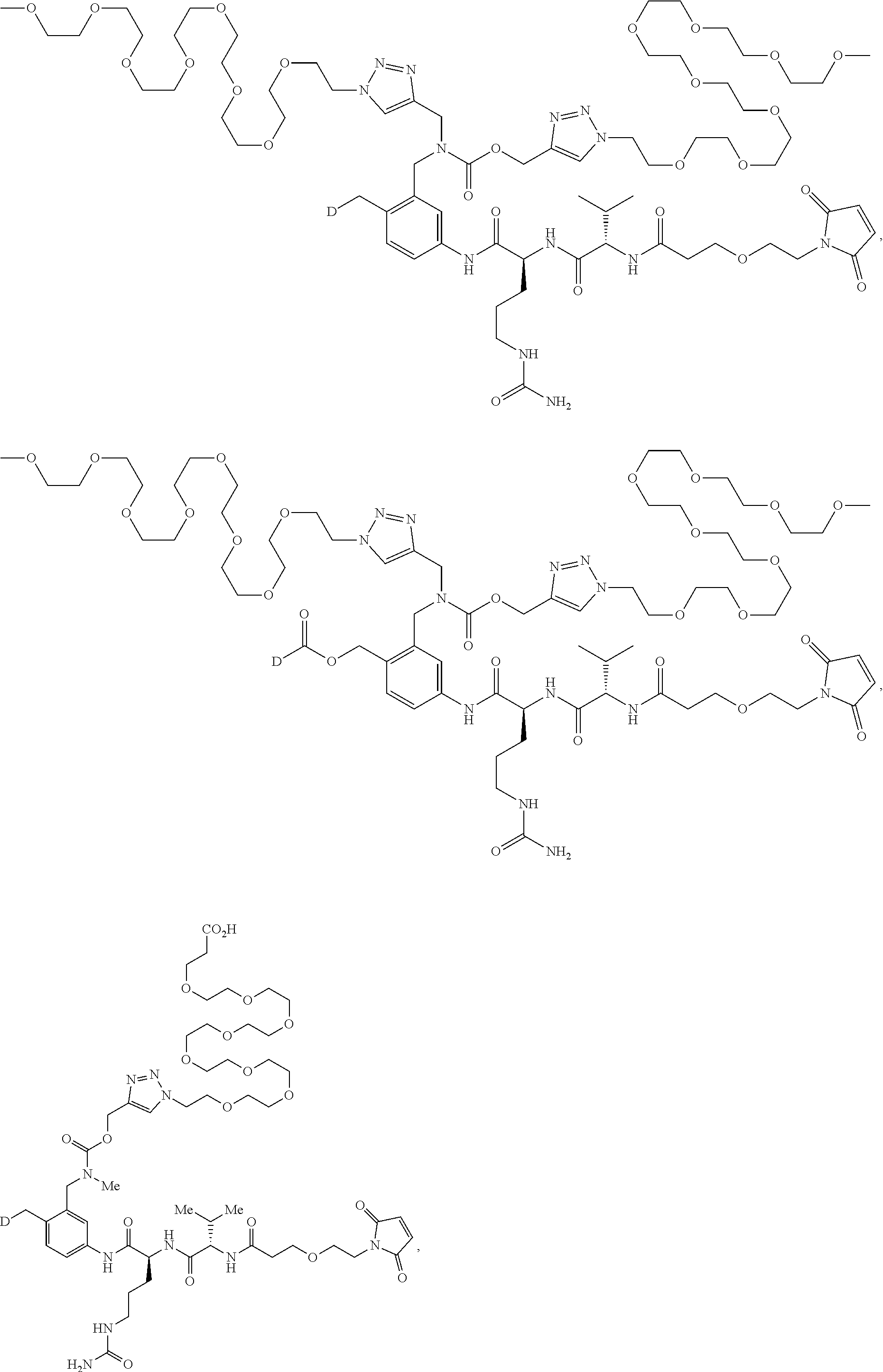 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(Cc1cn(CCOCCOCCOCCOCCOCCOCCOCCOC)nn1)C(=O)OCc1cn(CCOCCOCCOCCOCCOCCOCCOCCOC)nn1 ; [2H]C(=O)OCc1ccc(NC(=O)[C@H](CCCNC(N)=O)NC(=O)[C@@H](NC(=O)CCOCCN2C(=O)C=CC2=O)C(C)C)cc1CN(Cc1cn(CCOCCOCCOCCOCCOCCOCCOCCOC)nn1)C(=O)OCc1cn(CCOCCOCCOCCOCCOCCOCCOCCOC)nn1 ; [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)OCc1cn(CCOCCOCCOCCOCCOCCOCCOCCOCCC(=O)O)nn1]

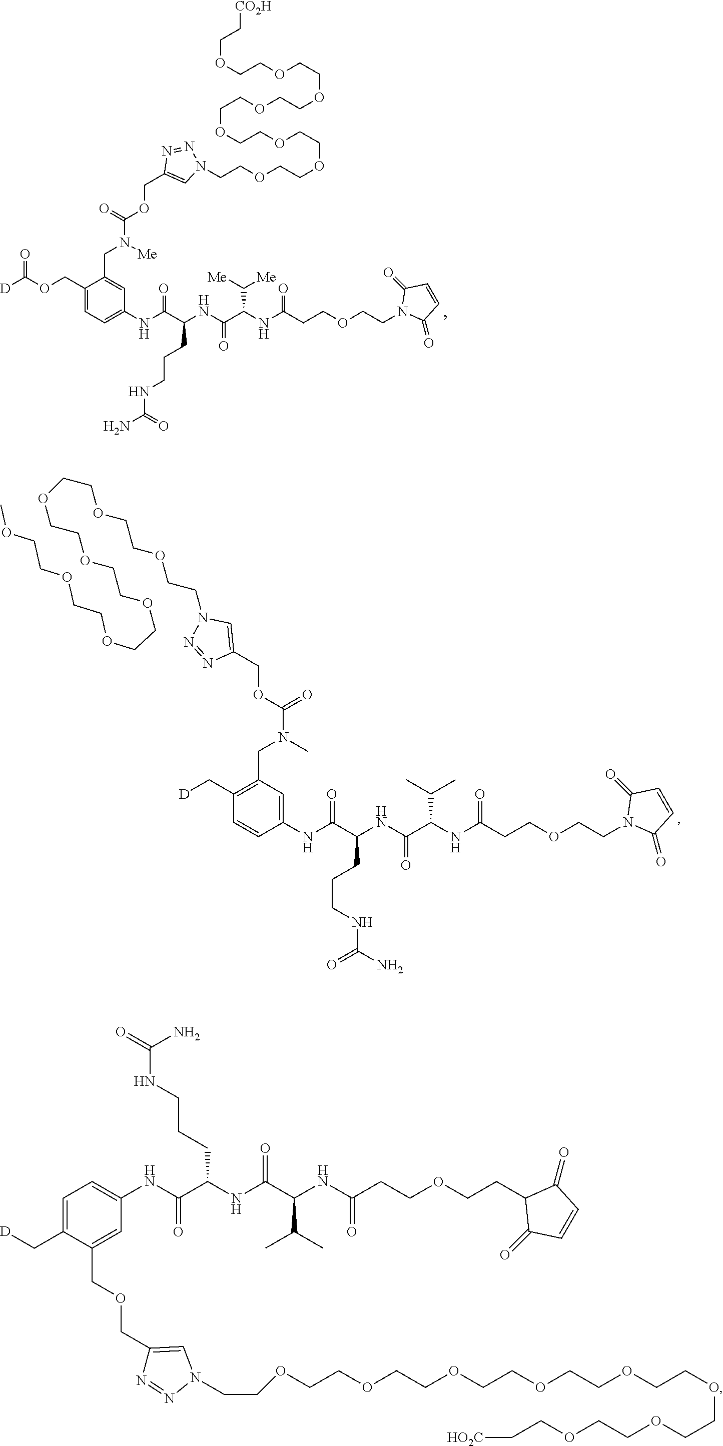 [SMILES: [2H]C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)OCc1cn(CCOCCOCCOCCOCCOCCOCCOCCOCCC(=O)O)nn1 ; [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)OCc1cn(CCOCCOCCOCCOCCOCCOCCOCCOC)nn1 ; [2H]Cc1ccc(NC(=O)[C@H](CCCNC(N)=O)NC(=O)[C@@H](NC(=O)CCOCCC2C(=O)C=CC2=O)C(C)C)cc1COCc1cn(CCOCCOCCOCCOCCOCCOCCOCCOCCC(=O)O)nn1]

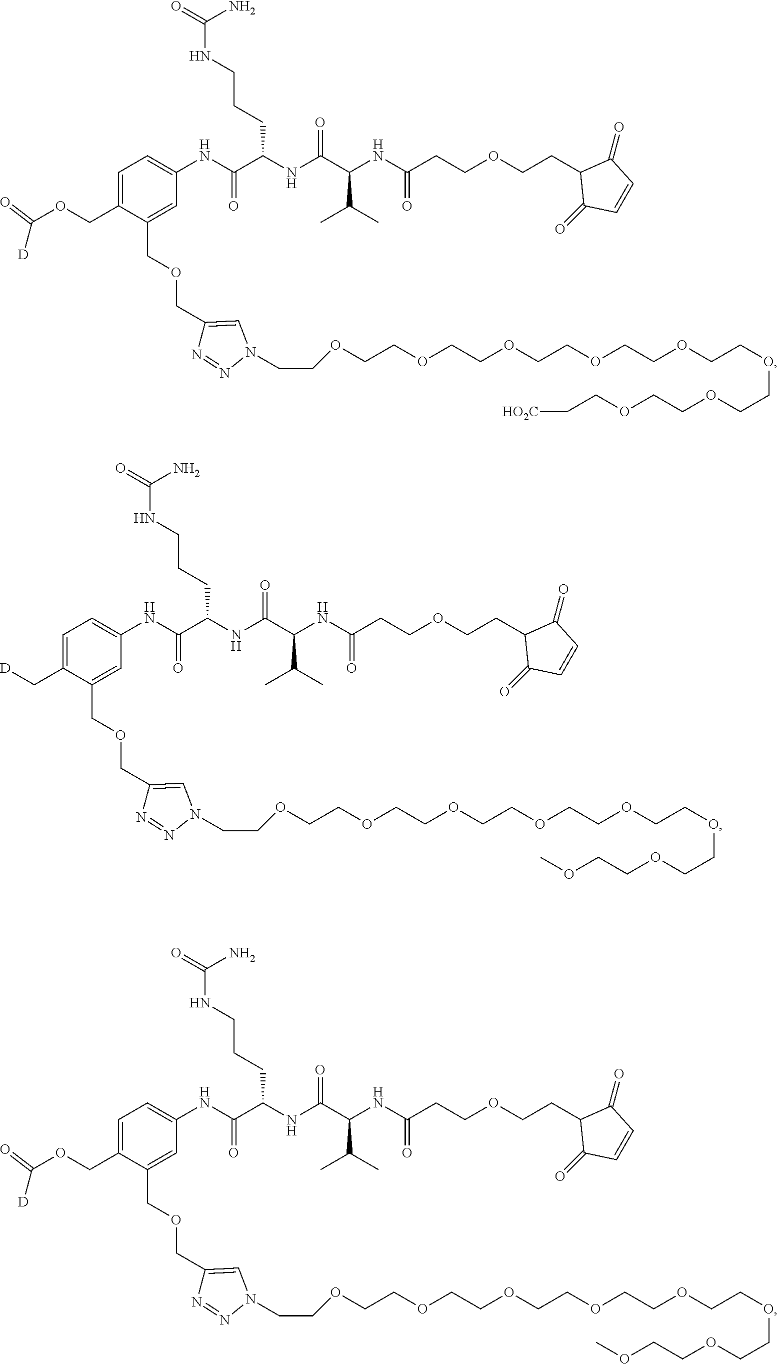 [SMILES: [2H]C(=O)OCc1ccc(NC(=O)[C@H](CCCNC(N)=O)NC(=O)[C@@H](NC(=O)CCOCCC2C(=O)C=CC2=O)C(C)C)cc1COCc1cn(CCOCCOCCOCCOCCOCCOCCOCCOCCC(=O)O)nn1 ; [2H]Cc1ccc(NC(=O)[C@H](CCCNC(N)=O)NC(=O)[C@@H](NC(=O)CCOCCC2C(=O)C=CC2=O)C(C)C)cc1COCc1cn(CCOCCOCCOCCOCCOCCOCCOCCOC)nn1 ; [2H]C(=O)OCc1ccc(NC(=O)[C@H](CCCNC(N)=O)NC(=O)[C@@H](NC(=O)CCOCCC2C(=O)C=CC2=O)C(C)C)cc1COCc1cn(CCOCCOCCOCCOCCOCCOCCOCCOC)nn1]

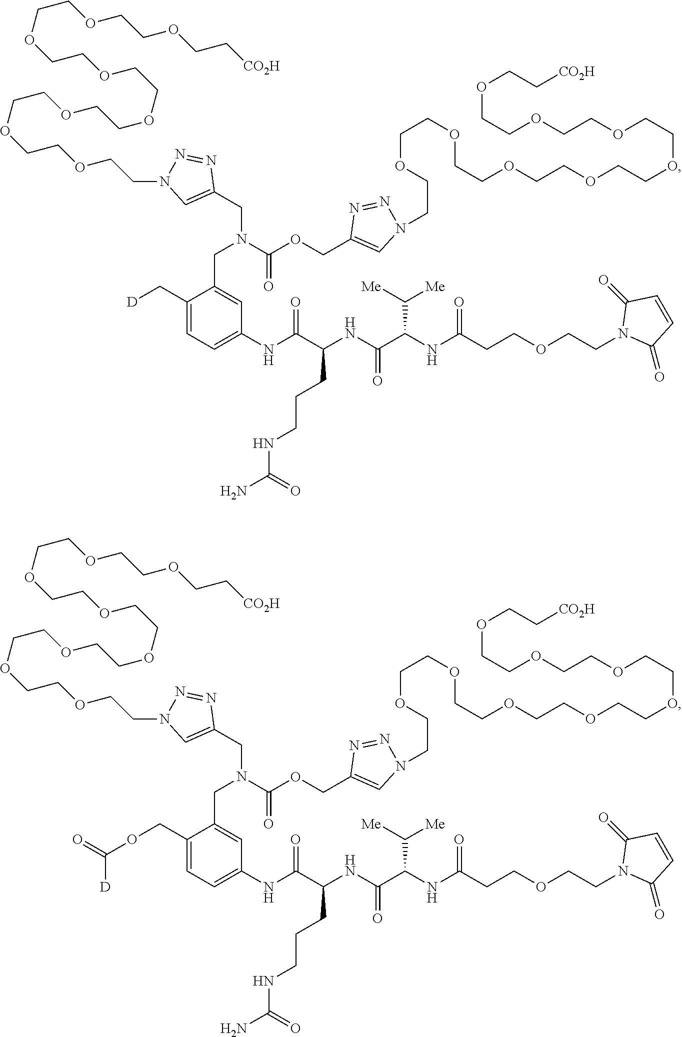 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(Cc1cn(CCOCCOCCOCCOCCOCCOCCOCCOCCC(=O)O)nn1)C(=O)OCc1cn(CCOCCOCCOCCOCCOCCOCCOCCOCCC(=O)O)nn1 ; [2H]C(=O)OCc1ccc(NC(=O)[C@H](CCCNC(N)=O)NC(=O)[C@@H](NC(=O)CCOCCN2C(=O)C=CC2=O)C(C)C)cc1CN(Cc1cn(CCOCCOCCOCCOCCOCCOCCOCCOCCC(=O)O)nn1)C(=O)OCc1cn(CCOCCOCCOCCOCCOCCOCCOCCOCCC(=O)O)nn1]

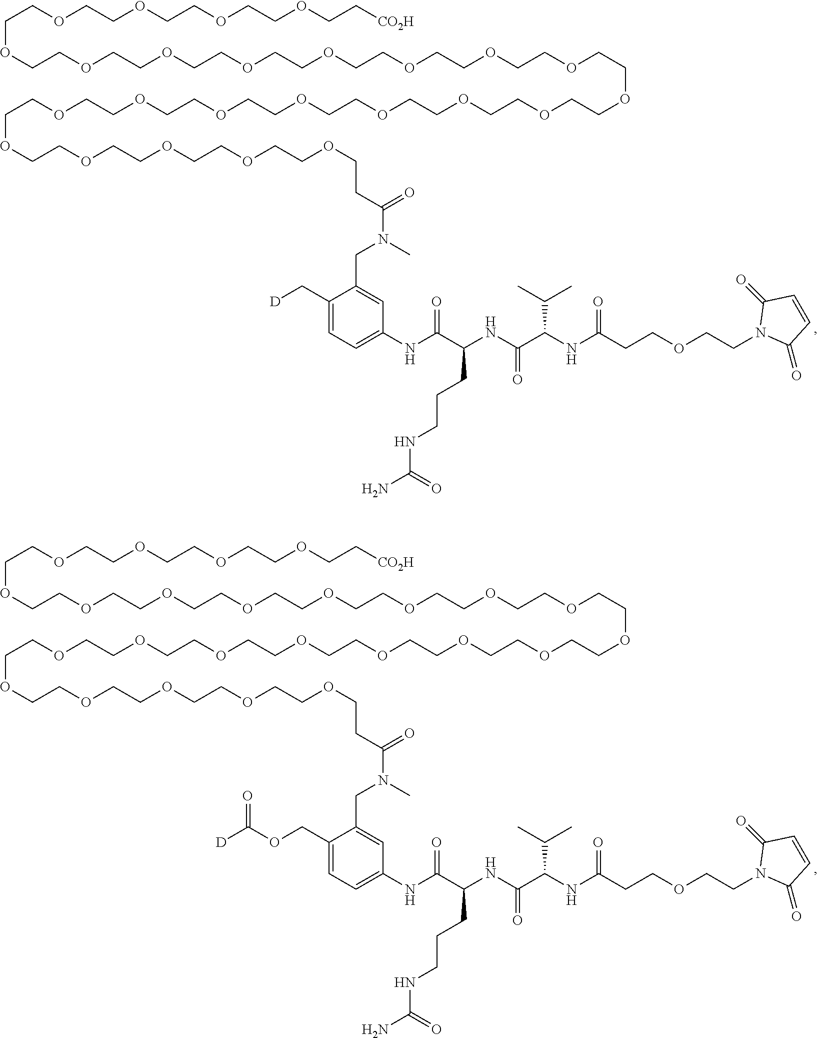 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O ; [2H]C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O]

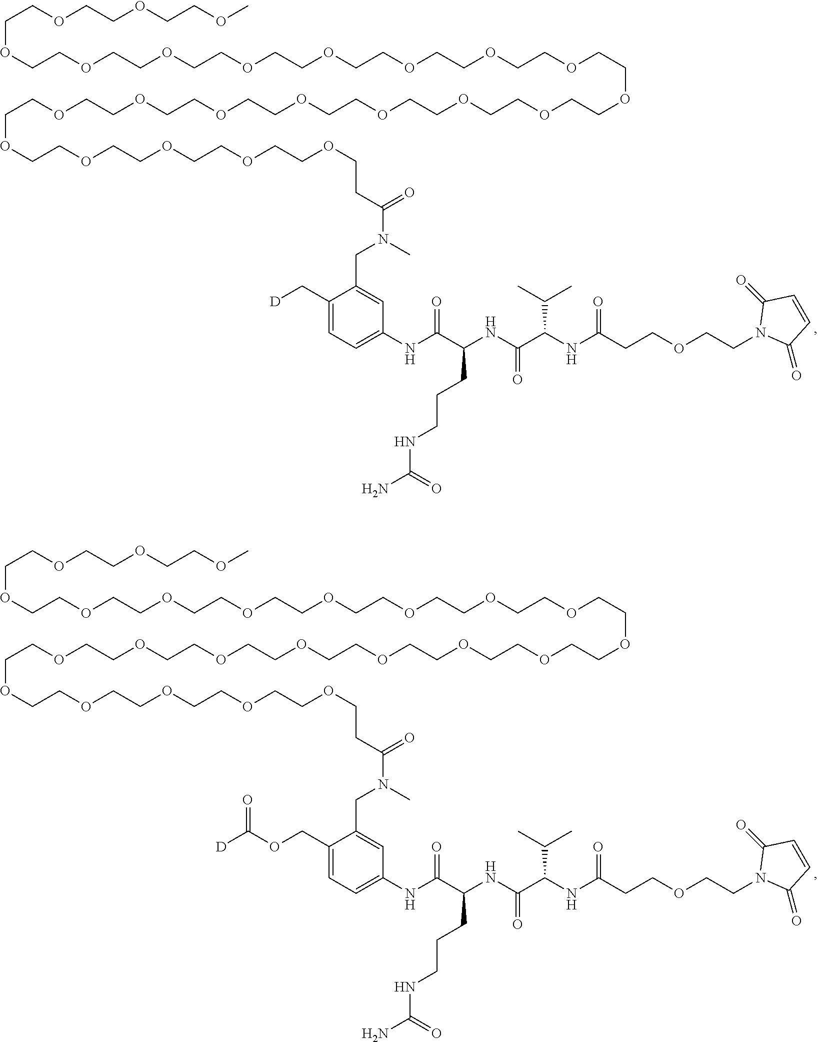 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCOCCOCCOCCOCCOCCOCCOCCOCCOCCOCCOCCOCCOCCOCCOCCOCCOCCOCCOCCOCCOCCOC ; [2H]C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCOCCOCCOCCOCCOCCOCCOCCOCCOCCOCCOCCOCCOCCOCCOCCOCCOCCOCCOCCOCCOCCOC]

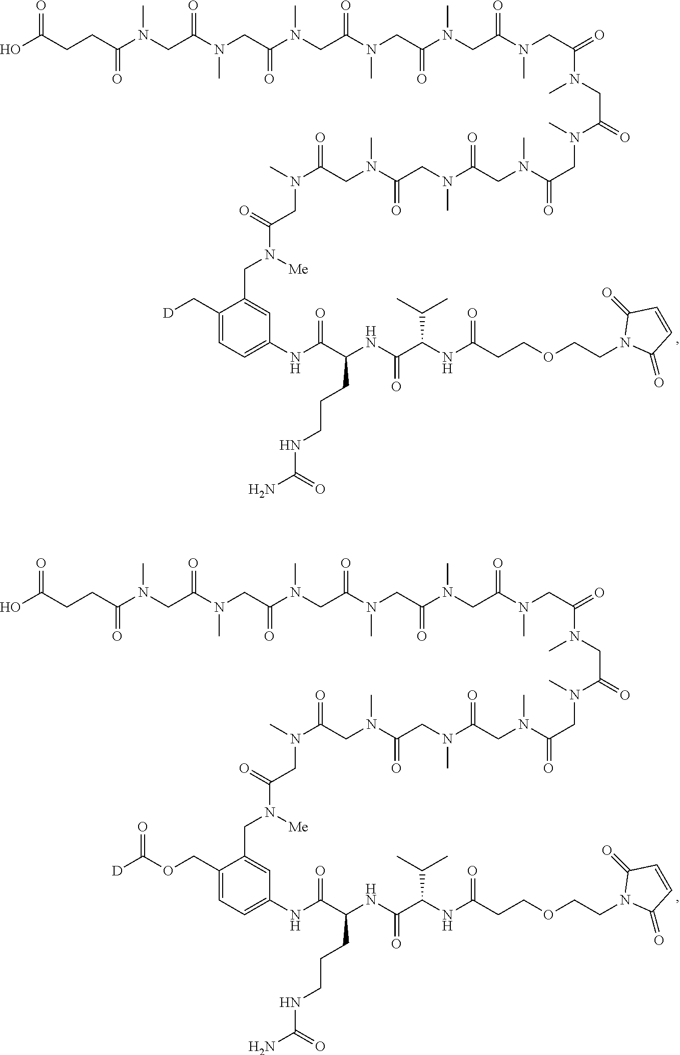 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O ; [2H]C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O]

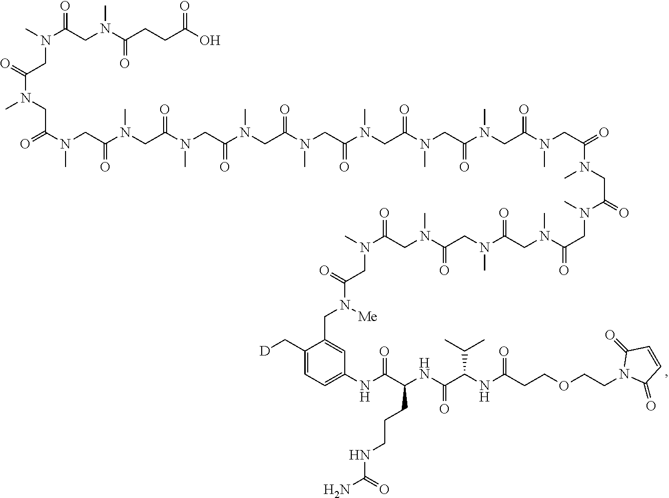 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O]

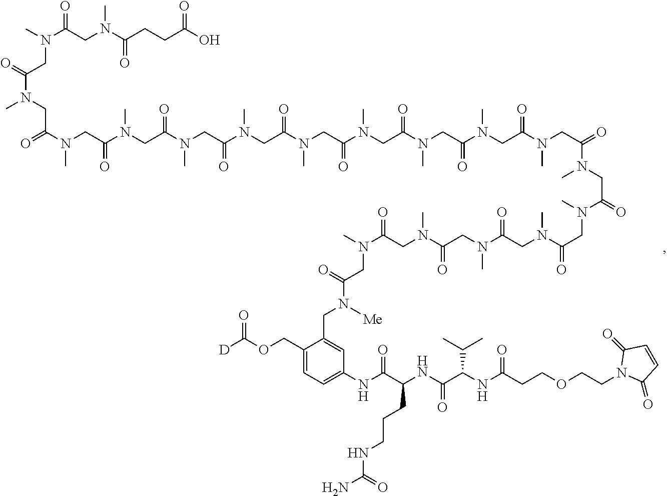 [SMILES: [2H]C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O]

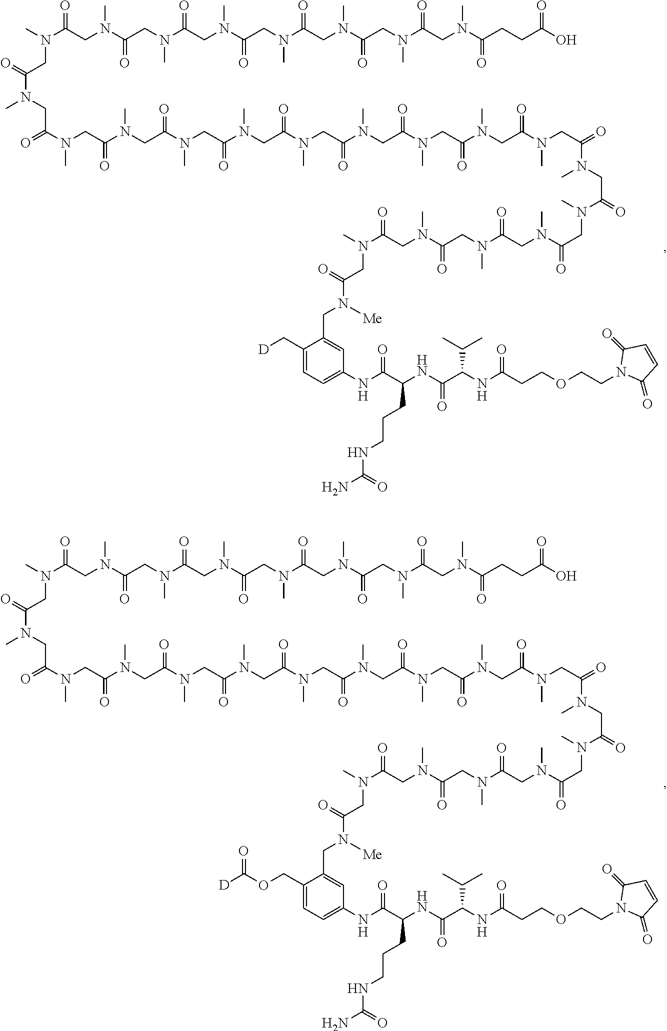

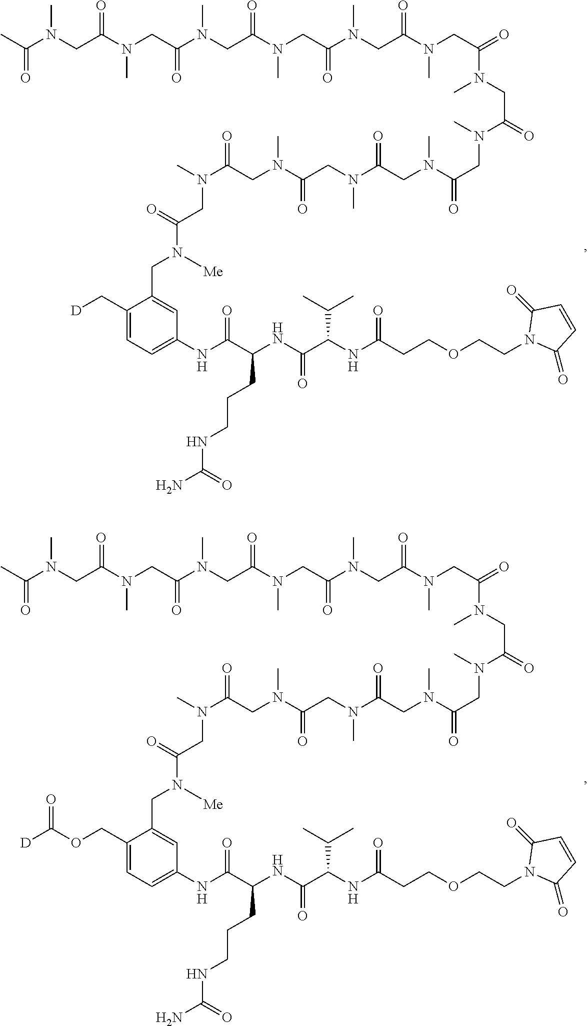 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O ; [2H]C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O]

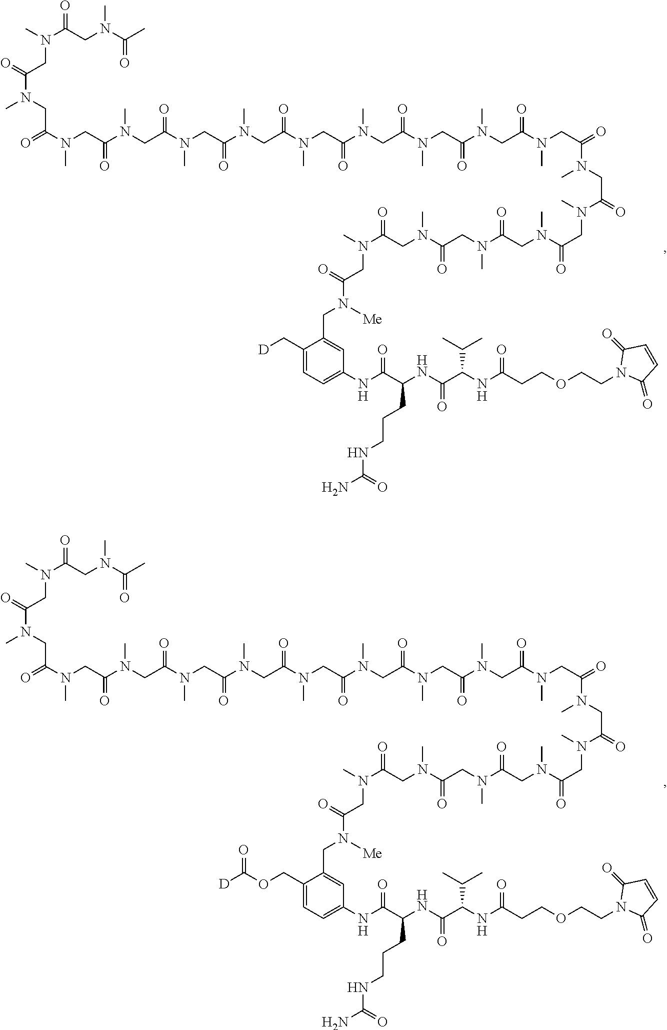

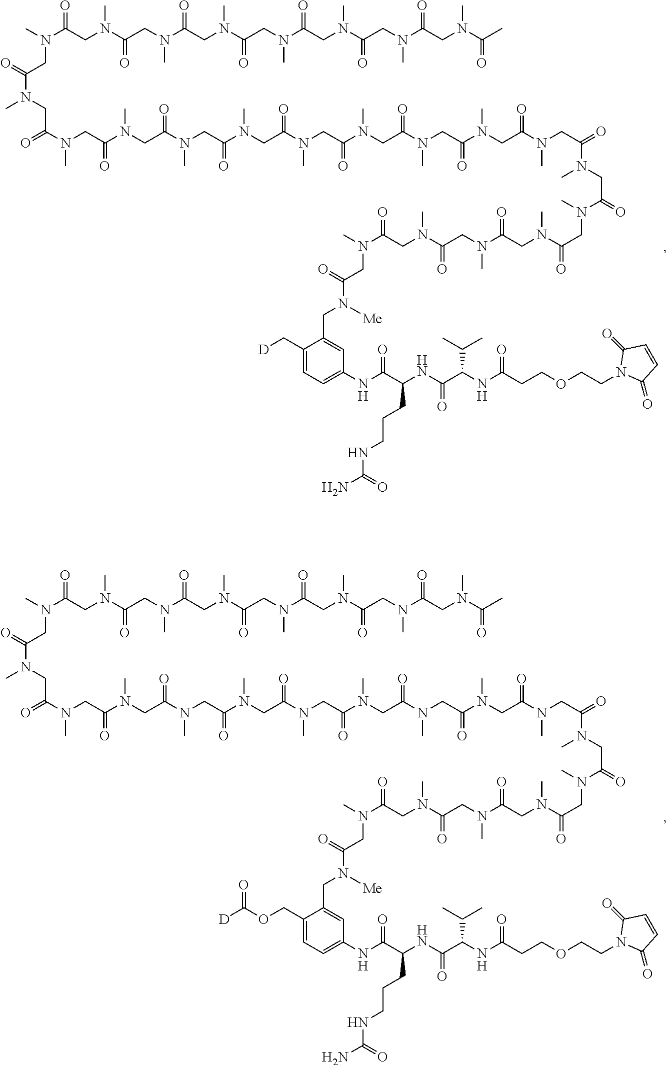

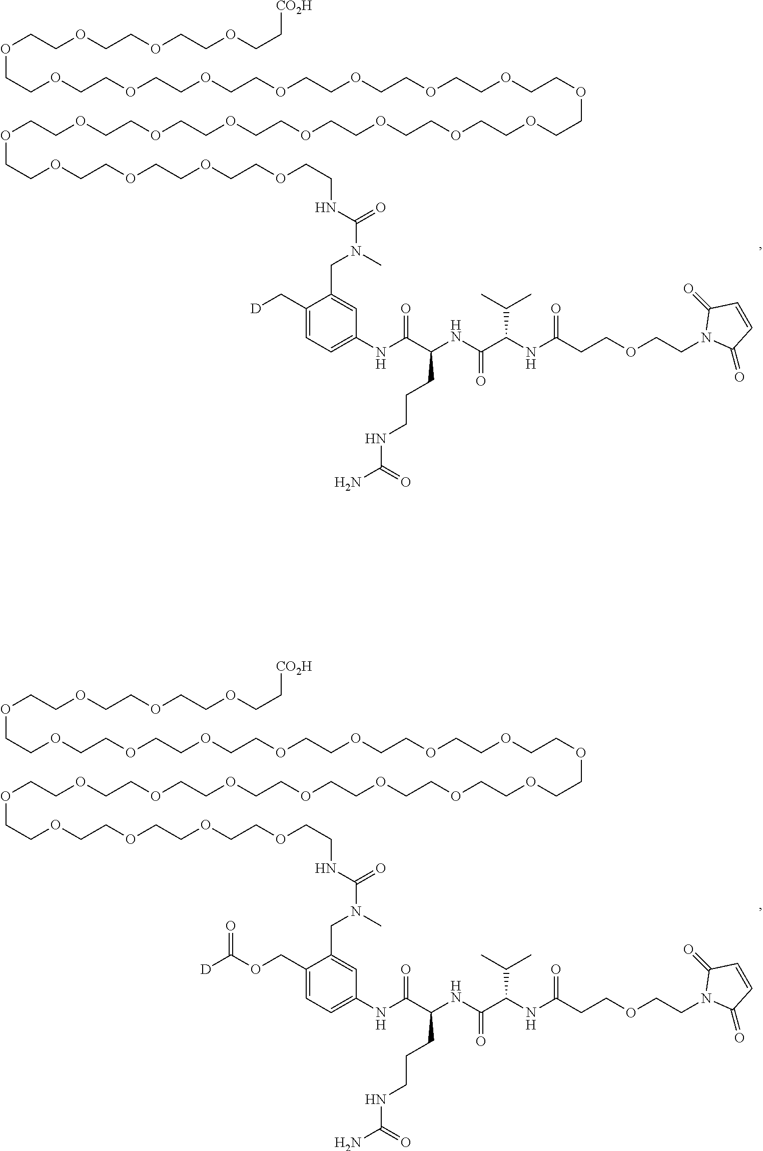 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O ; [2H]C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O]

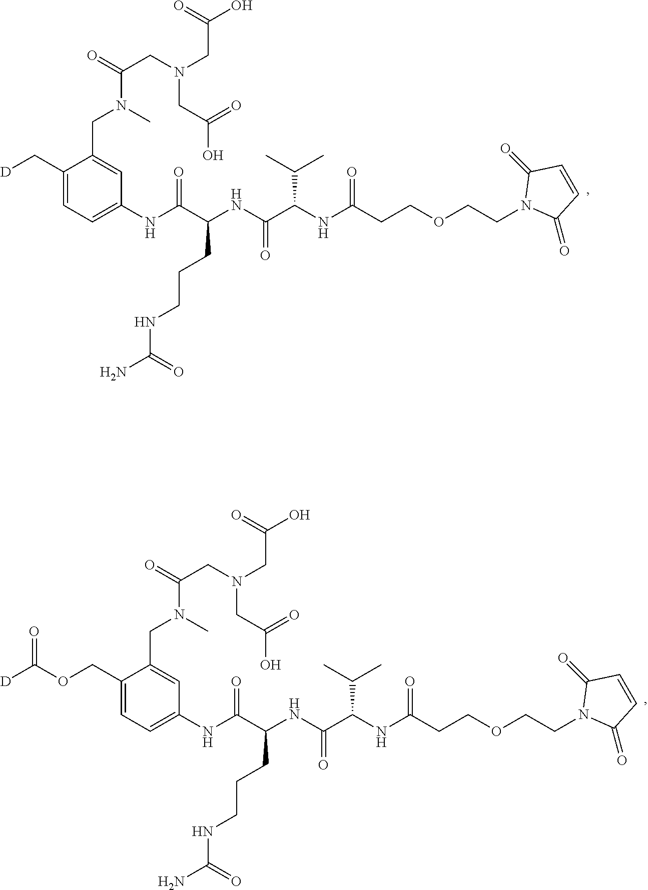 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)CN(CC(=O)O)CC(=O)O ; [2H]C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CN(CC(=O)O)CC(=O)O]

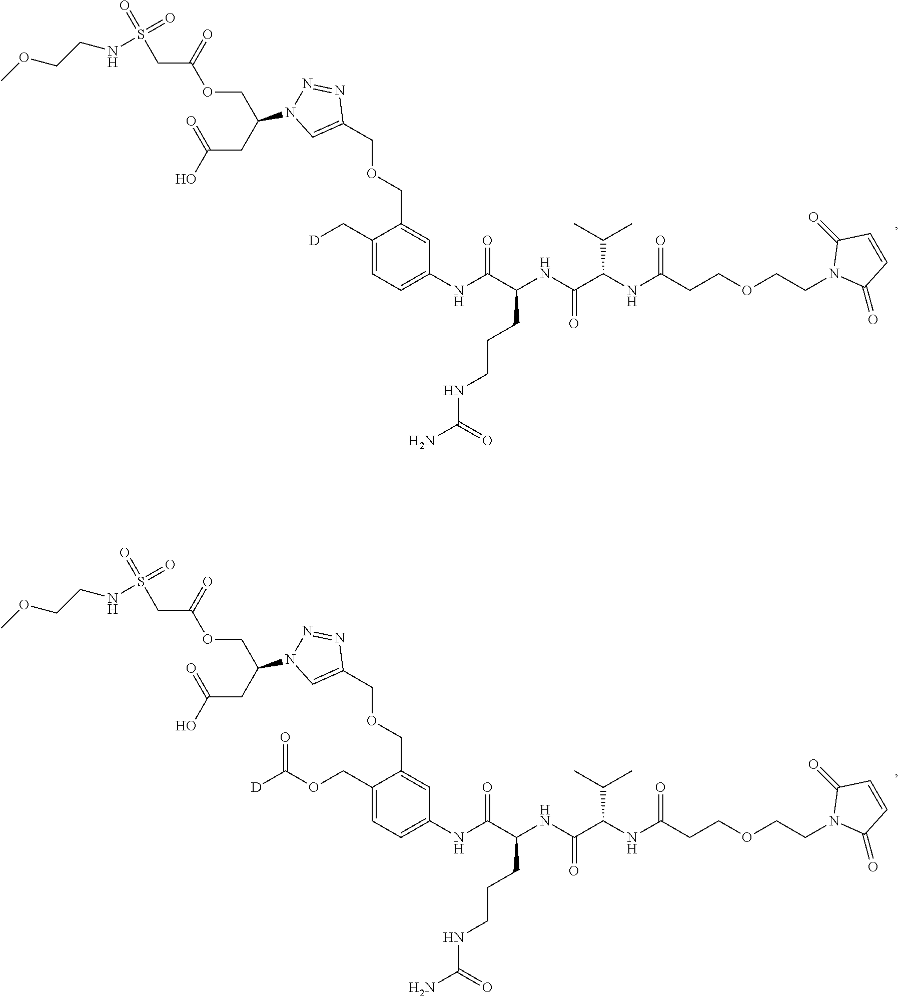 [SMILES: [2H]Cc1ccc(NC(=O)[C@H](CCCNC(N)=O)NC(=O)[C@@H](NC(=O)CCOCCN2C(=O)C=CC2=O)C(C)C)cc1COCc1cn([C@H](COC(=O)CS(=O)(=O)NCCOC)CC(=O)O)nn1 ; [2H]C(=O)OCc1ccc(NC(=O)[C@H](CCCNC(N)=O)NC(=O)[C@@H](NC(=O)CCOCCN2C(=O)C=CC2=O)C(C)C)cc1COCc1cn([C@H](COC(=O)CS(=O)(=O)NCCOC)CC(=O)O)nn1]

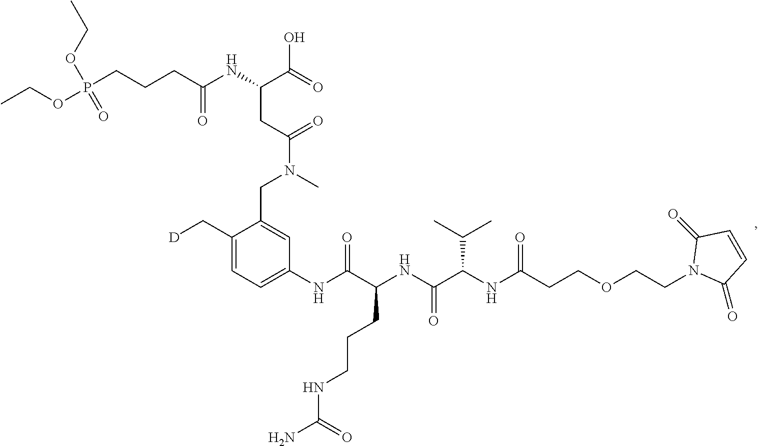 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)C[C@H](NC(=O)CCCP(=O)(OCC)OCC)C(=O)O]

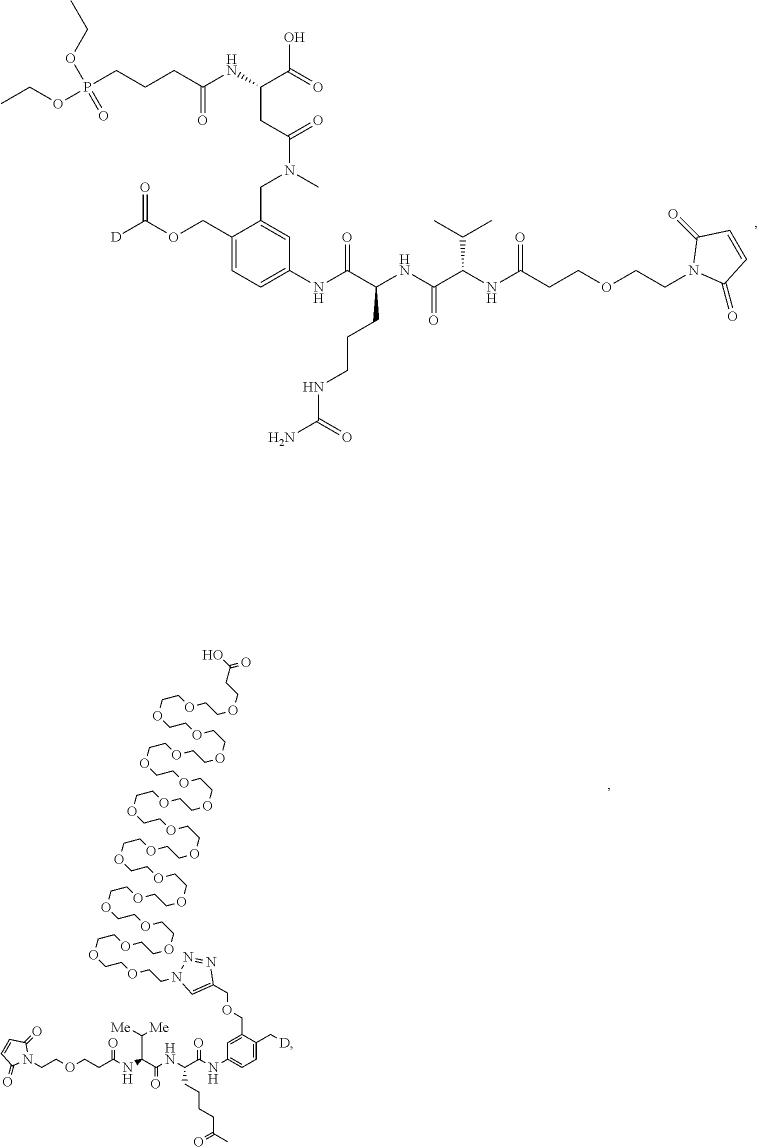

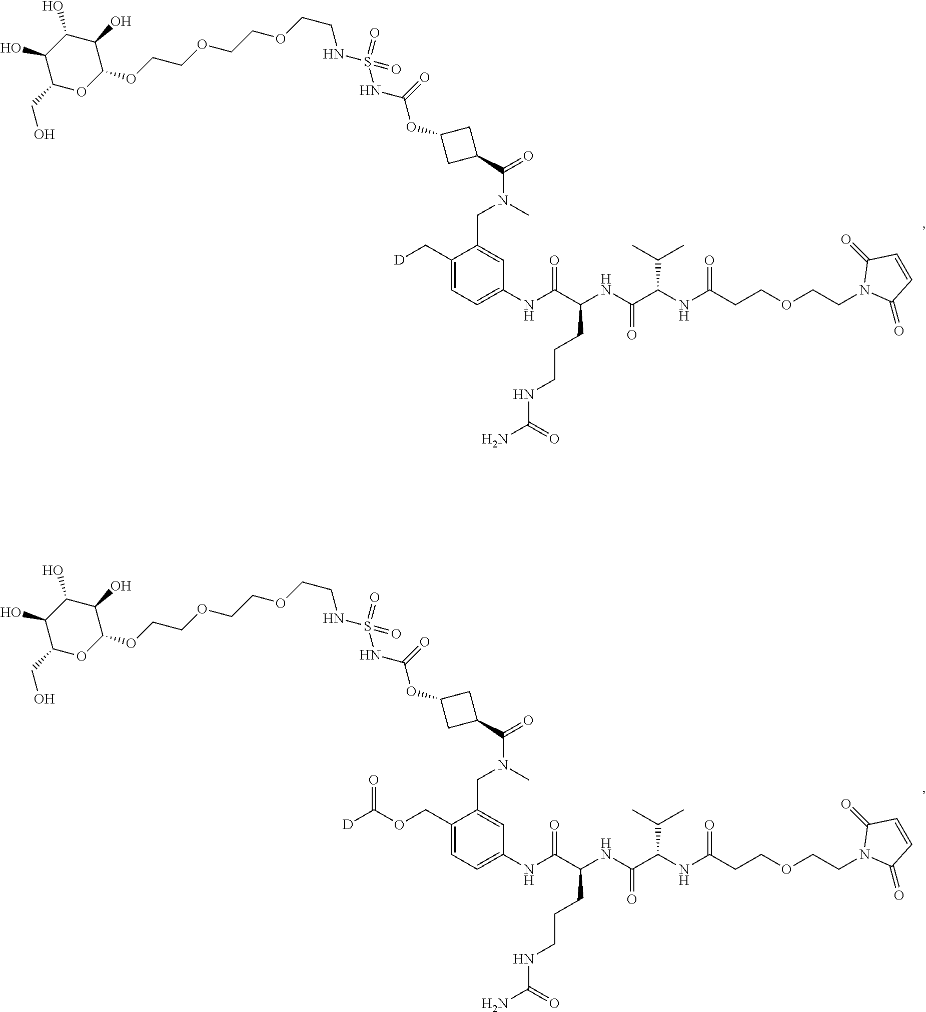 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)[C@H]1C[C@H](OC(=O)NS(=O)(=O)NCCOCCOCCO[C@@H]2O[C@H](CO)[C@@H](O)[C@H](O)[C@H]2O)C1 ; [2H]C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)[C@H]1C[C@H](OC(=O)NS(=O)(=O)NCCOCCOCCO[C@@H]2O[C@H](CO)[C@@H](O)[C@H](O)[C@H]2O)C1]

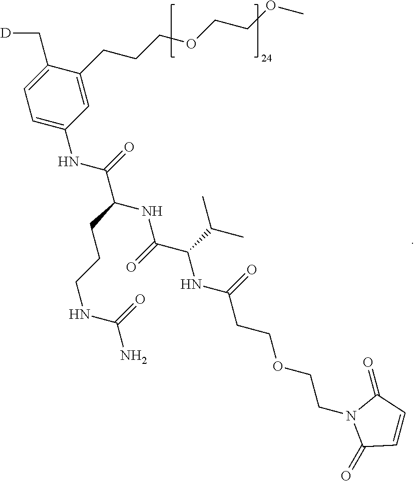

In some embodiments, the antibody-drug conjugate comprises the linker-drug group, -(L-D), which comprises a formula selected from:

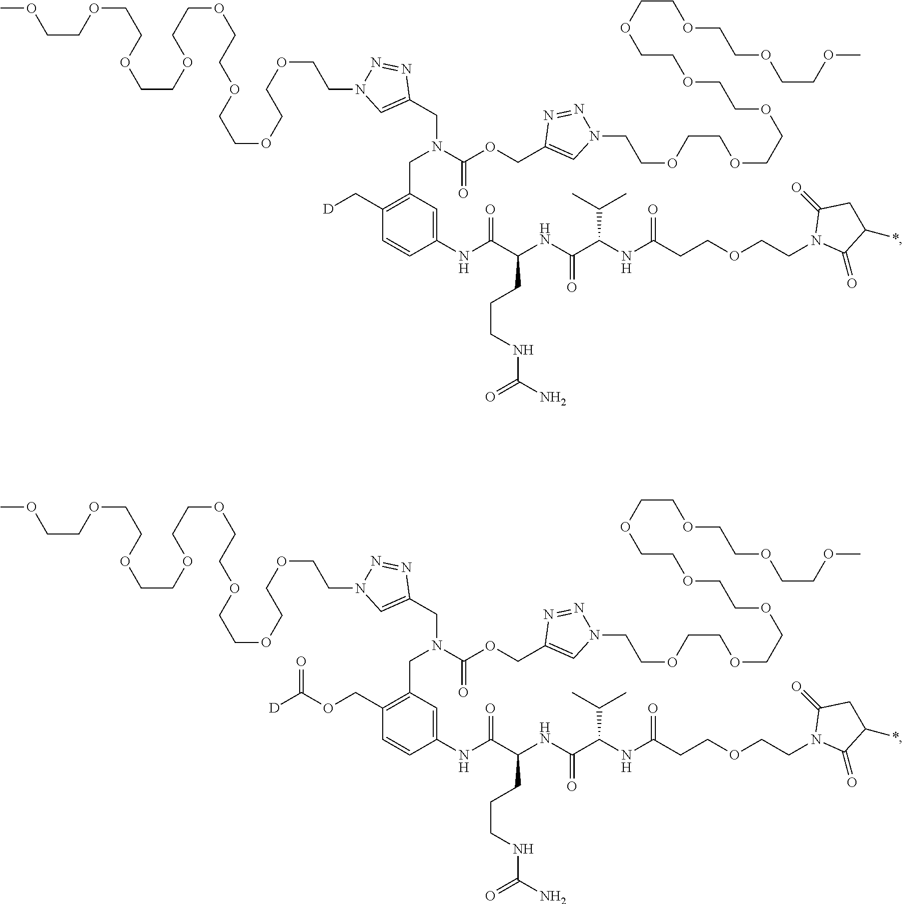

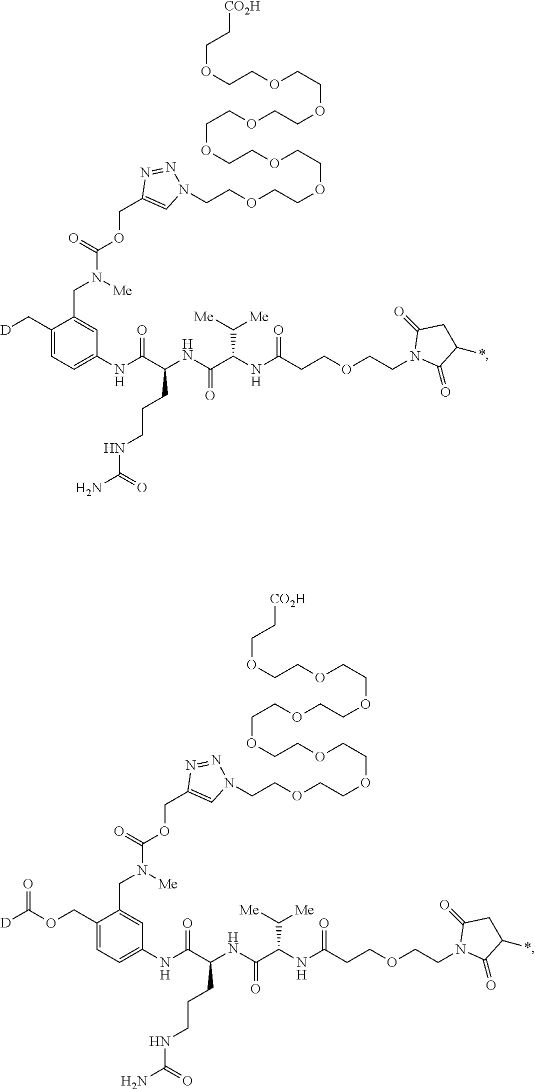

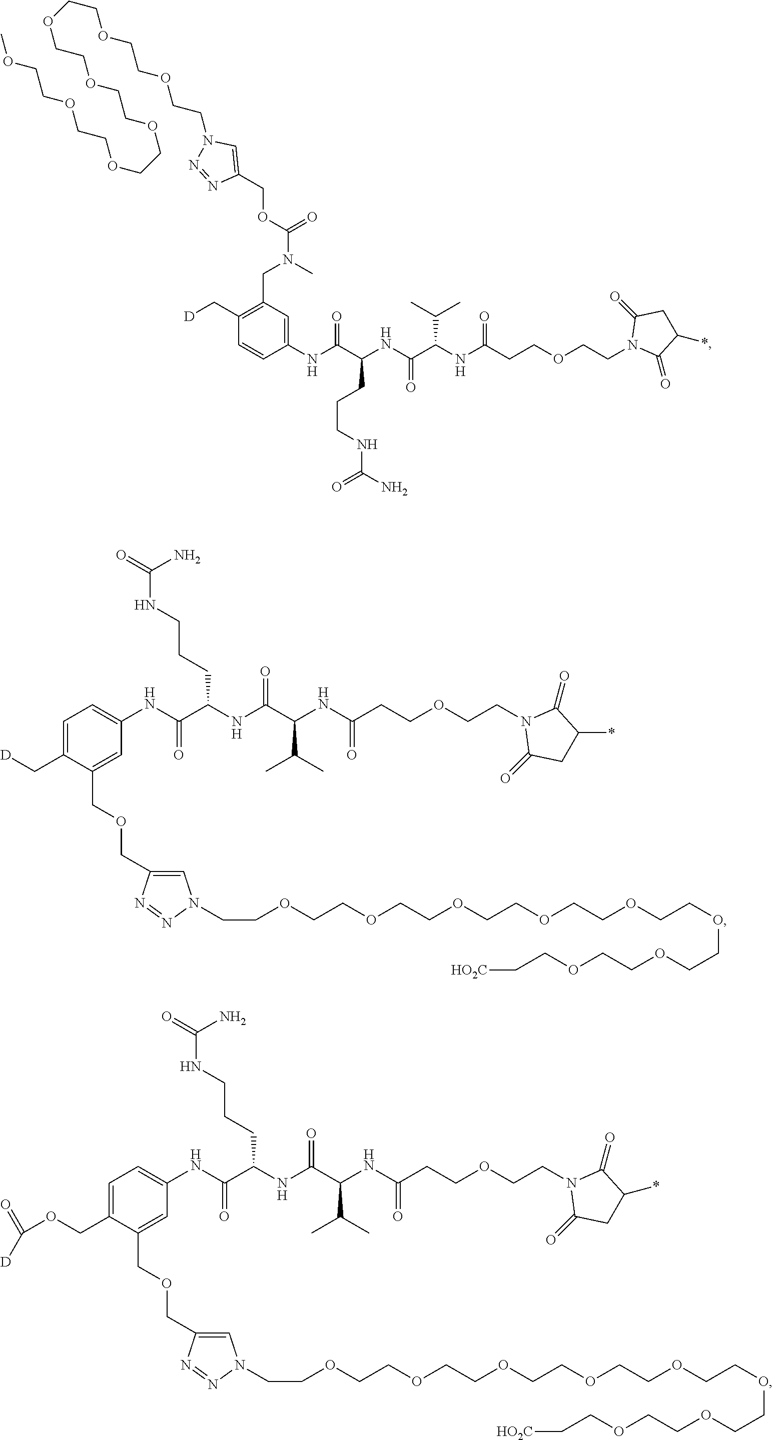

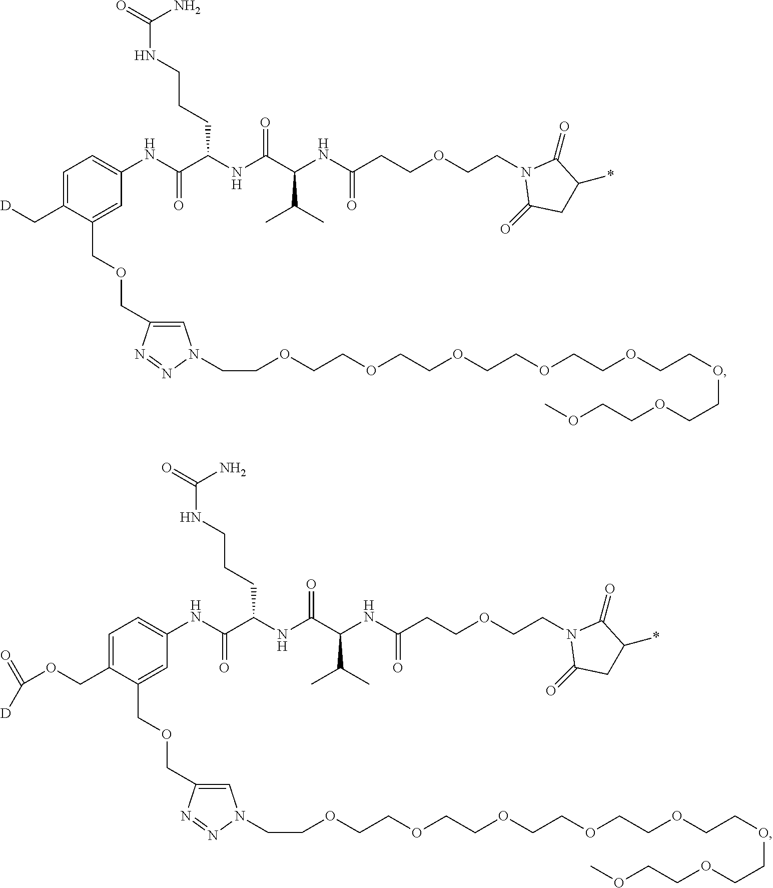 [SMILES: *C1CC(=O)N(CCOCCC(=O)N[C@H](C(=O)N[C@@H](CCCNC(N)=O)C(=O)Nc2ccc(C[2H])c(COCc3cn(CCOCCOCCOCCOCCOCCOCCOCCOC)nn3)c2)C(C)C)C1=O ; *C1CC(=O)N(CCOCCC(=O)N[C@H](C(=O)N[C@@H](CCCNC(N)=O)C(=O)Nc2ccc(COC([2H])=O)c(COCc3cn(CCOCCOCCOCCOCCOCCOCCOCCOC)nn3)c2)C(C)C)C1=O]

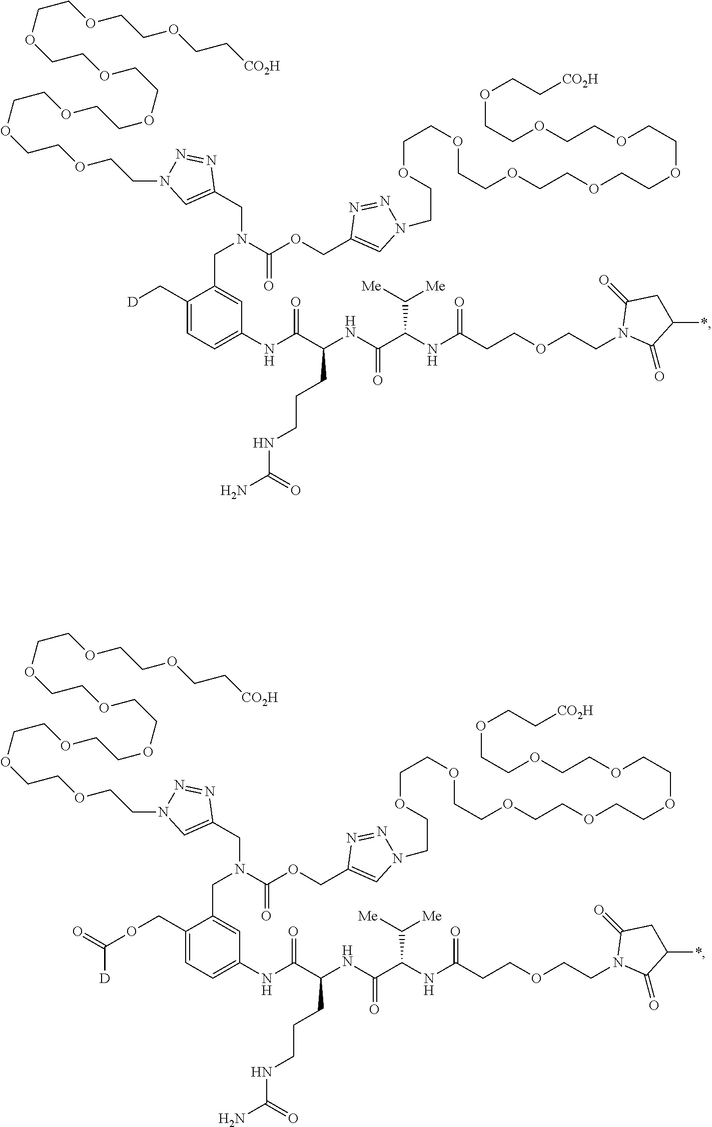

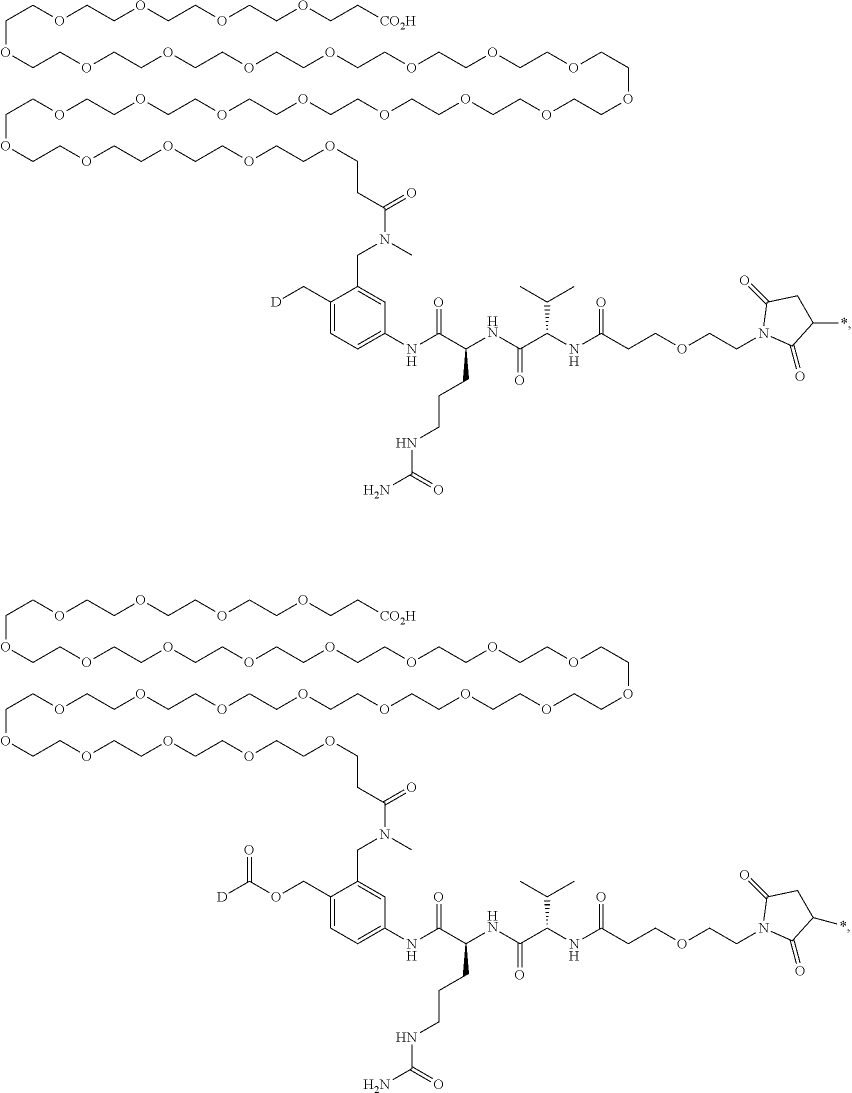

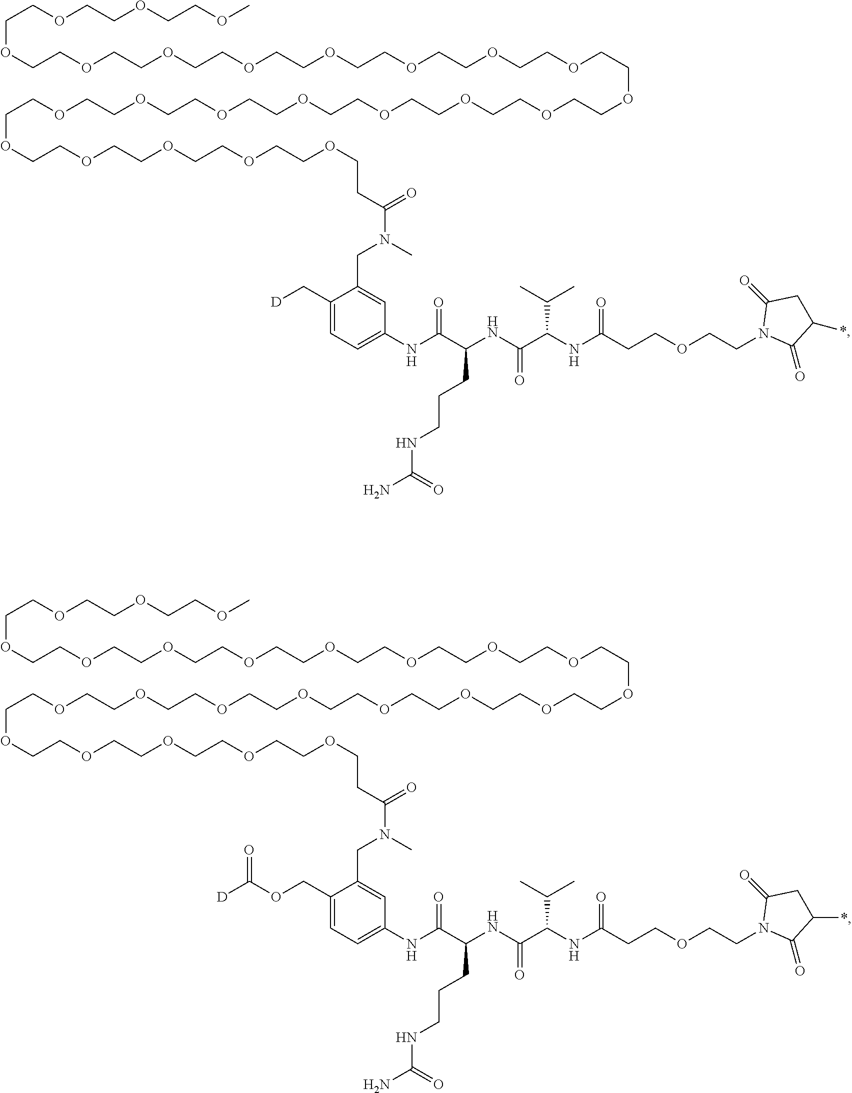

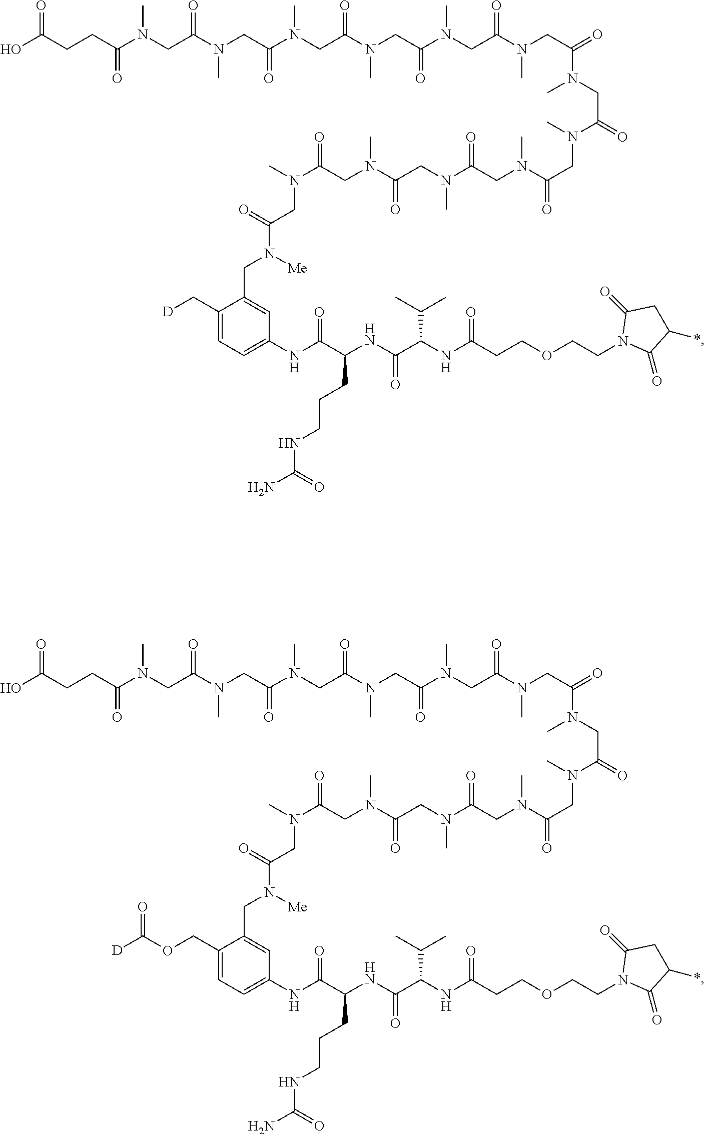

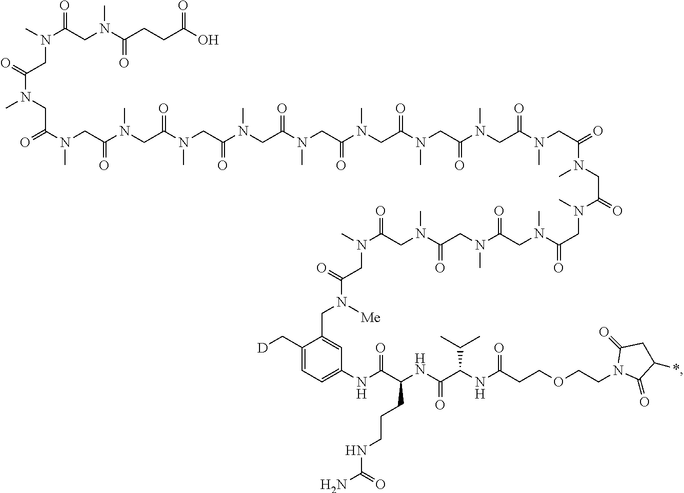

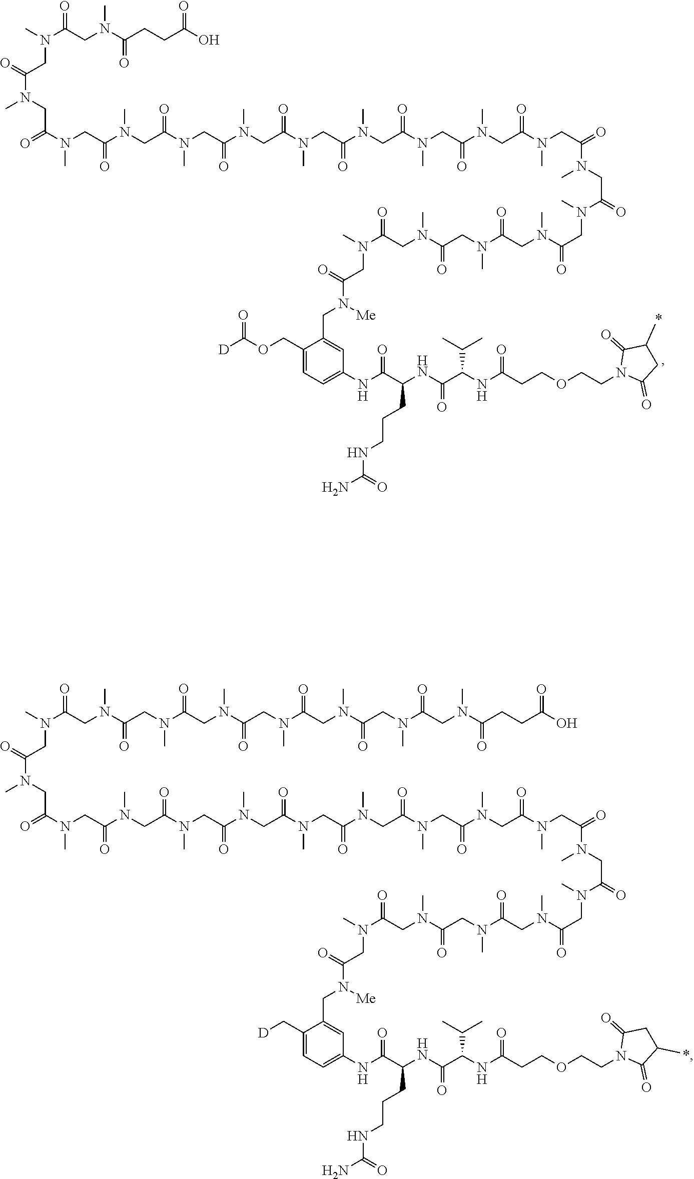

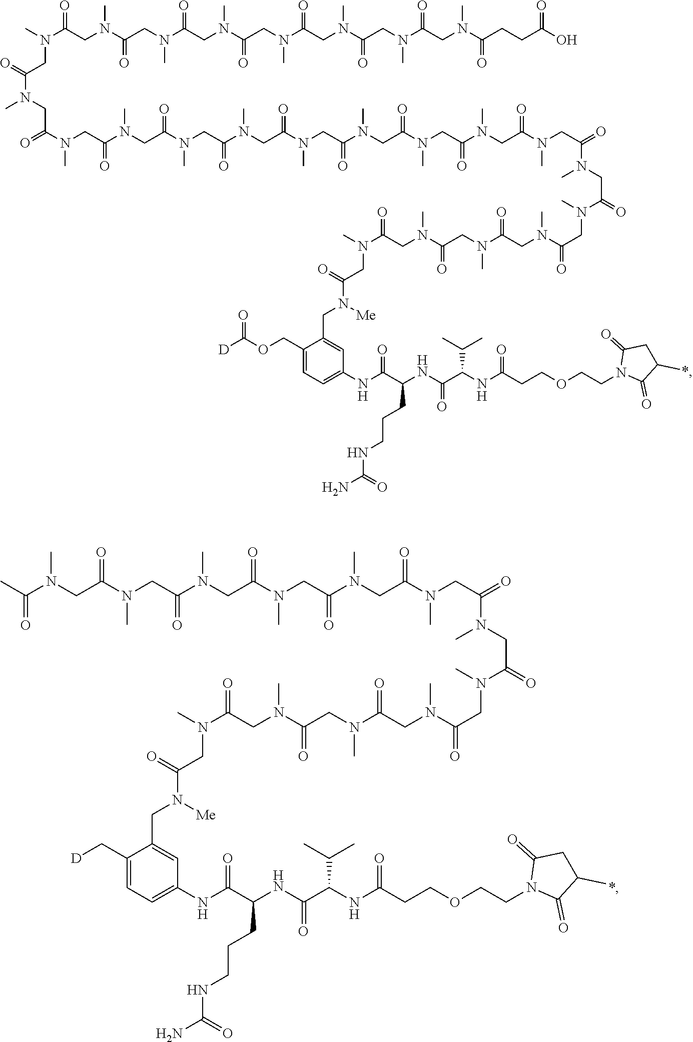

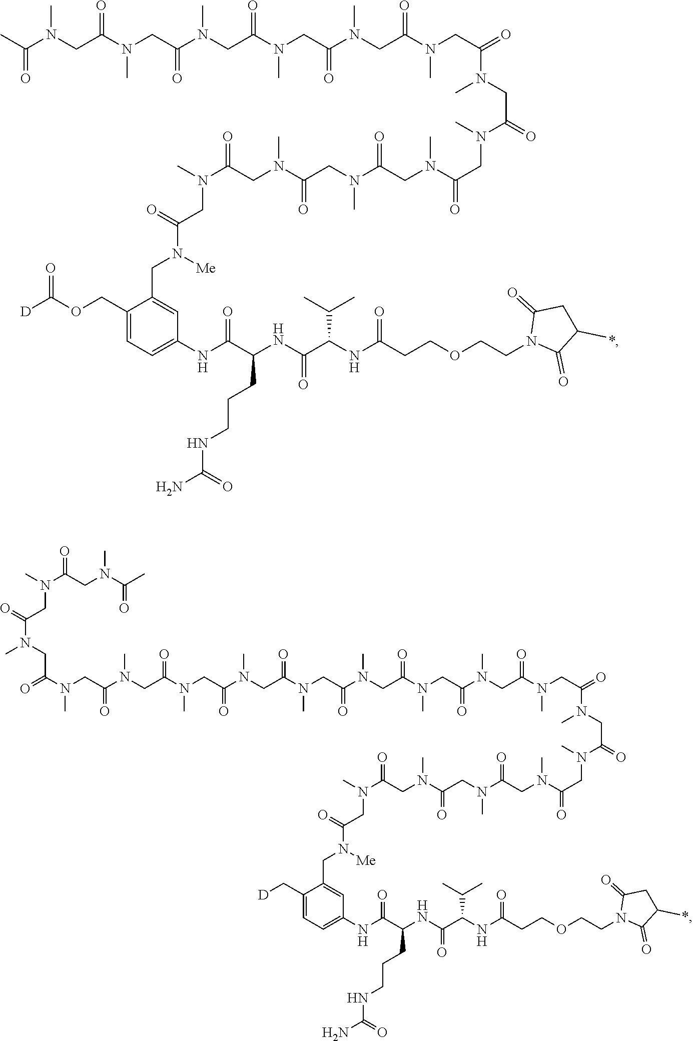 [SMILES: [2H]C(=O)OCc1ccc(NC(=O)[C@H](CCCNC(N)=O)NC(=O)[C@@H](NC(=O)CCOCCN2C(=O)CC(C)C2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O ; [2H]Cc1ccc(NC(=O)[C@H](CCCNC(N)=O)NC(=O)[C@@H](NC(=O)CCOCCN2C(=O)CC(C)C2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O]

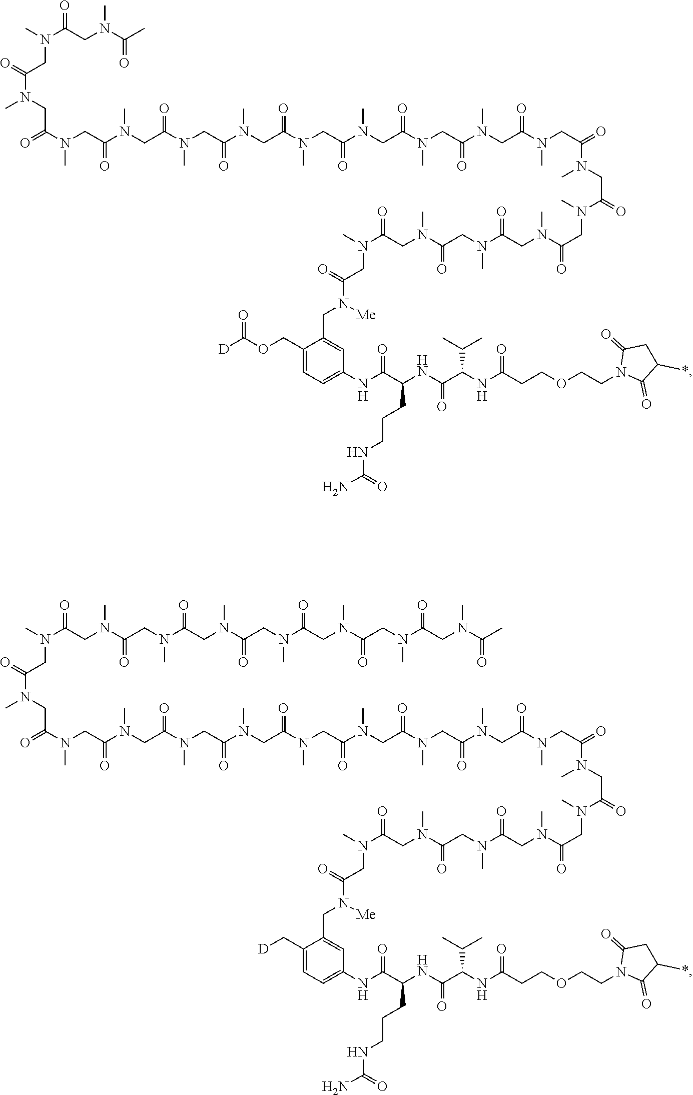

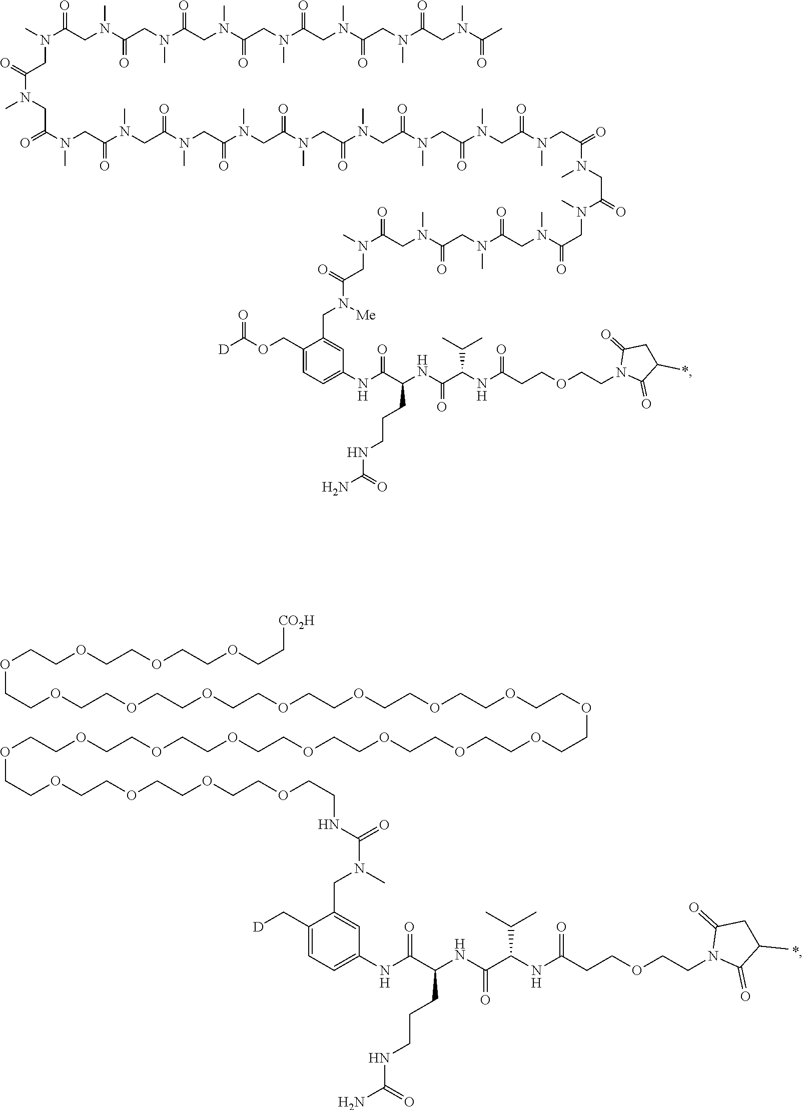

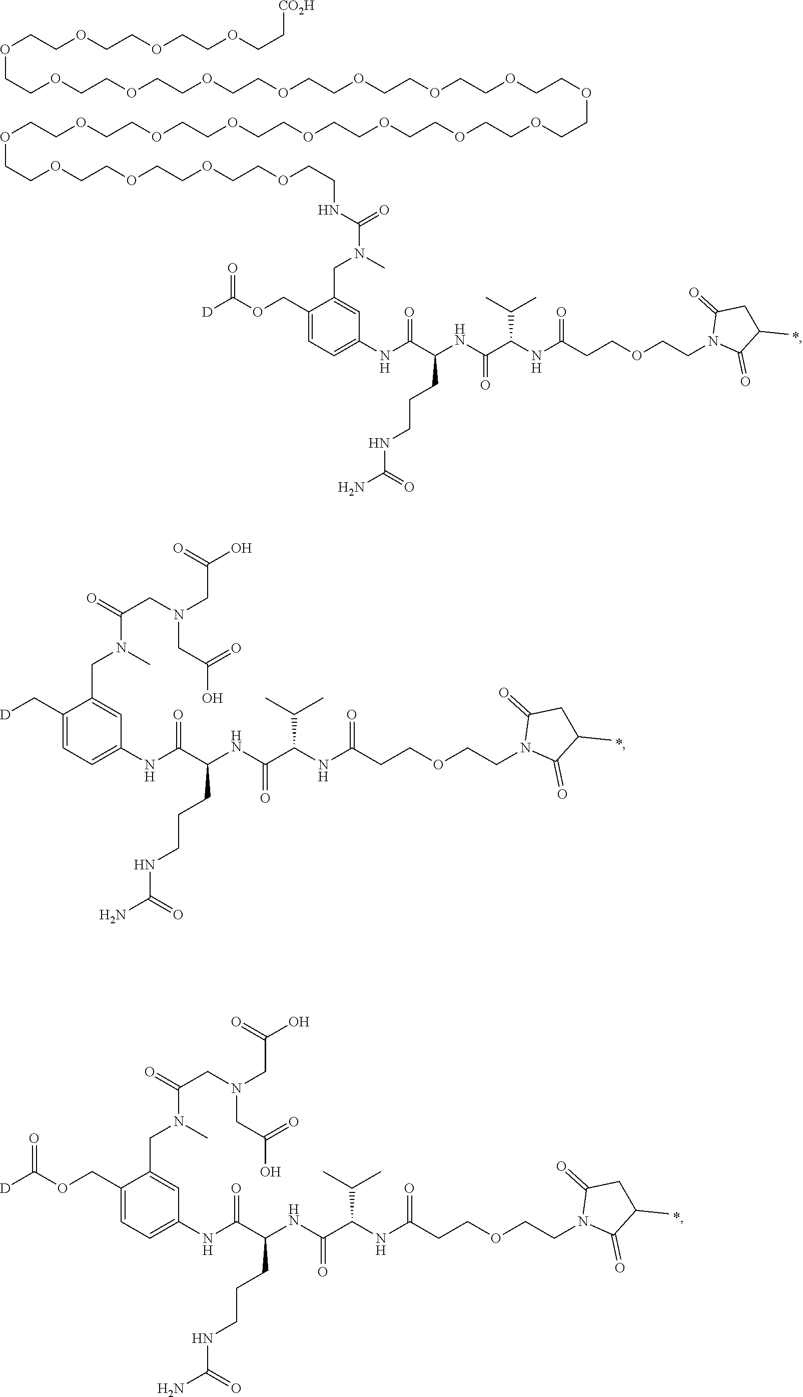 [SMILES: [2H]C(=O)OCc1ccc(NC(=O)[C@H](CCCNC(N)=O)NC(=O)[C@@H](NC(=O)CCOCCN2C(=O)CC(C)C2=O)C(C)C)cc1CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O ; [2H]Cc1ccc(NC(=O)[C@H](CCCNC(N)=O)NC(=O)[C@@H](NC(=O)CCOCCN2C(=O)CC(C)C2=O)C(C)C)cc1CN(C)C(=O)CN(CC(=O)O)CC(=O)O ; [2H]C(=O)OCc1ccc(NC(=O)[C@H](CCCNC(N)=O)NC(=O)[C@@H](NC(=O)CCOCCN2C(=O)CC(C)C2=O)C(C)C)cc1CN(C)C(=O)CN(CC(=O)O)CC(=O)O]

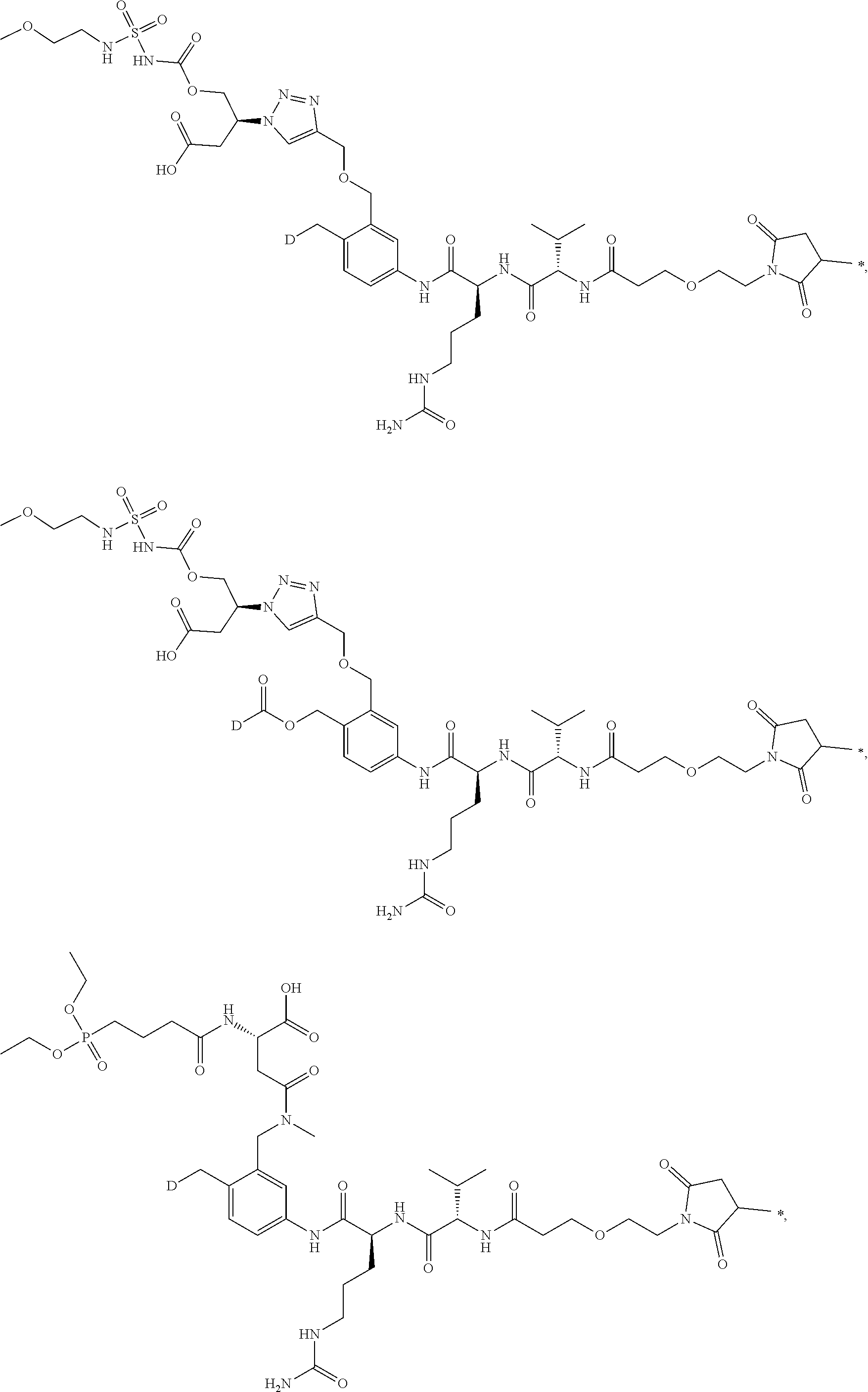

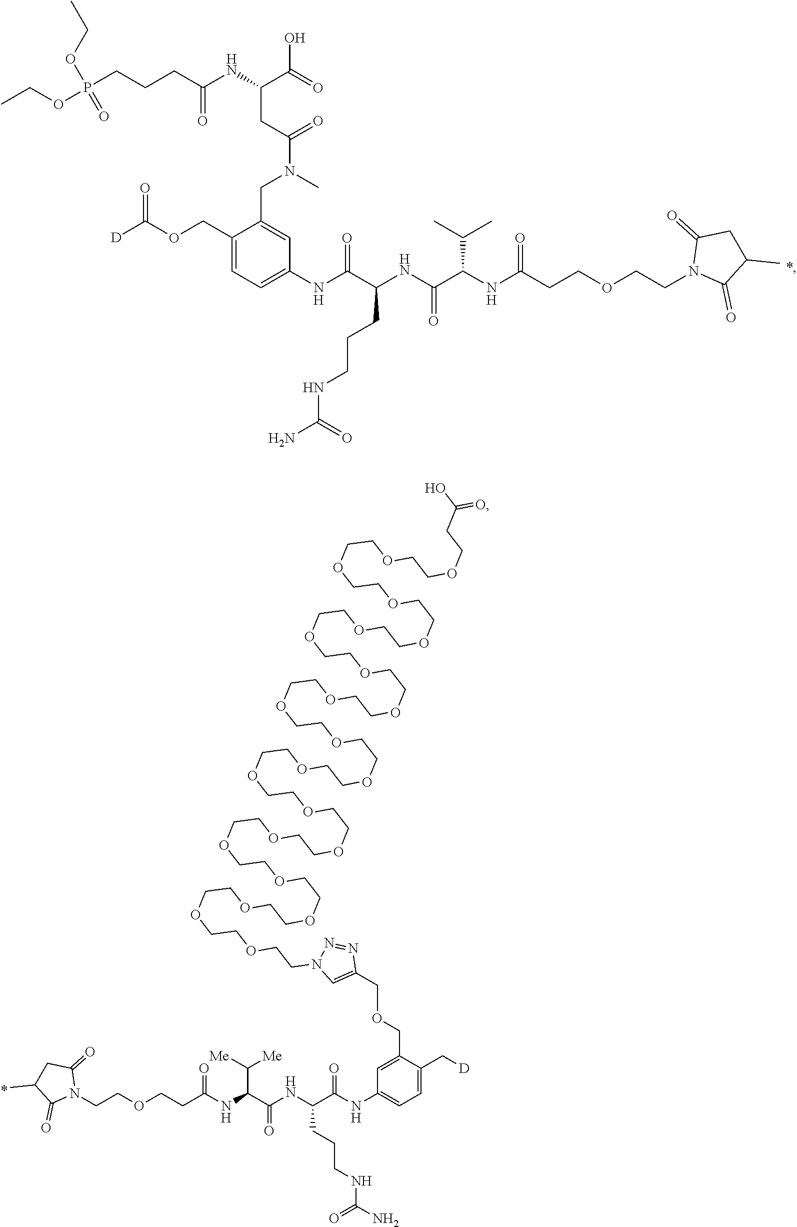

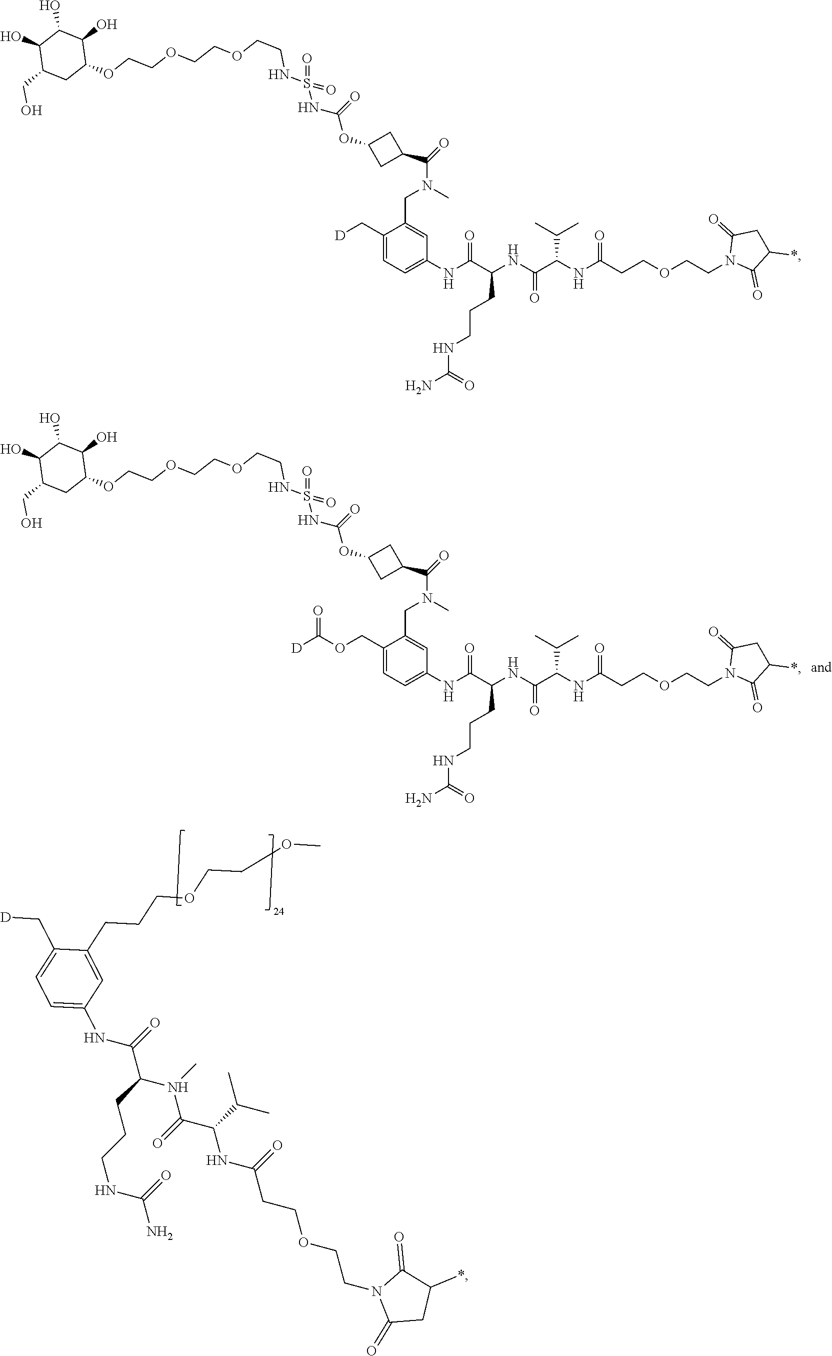

and wherein —* is a bond to the antibody or antigen-binding fragment thereof.

In some embodiments, the panRAS inhibitor (0) comprises a compound of Formula (Ia):

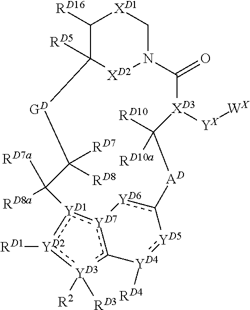

or a pharmaceutically acceptable salt thereof, wherein the variables are described above for Formula (Ia).

In some embodiments, the panRAS inhibitor (D) comprises a compound of Formula (I):

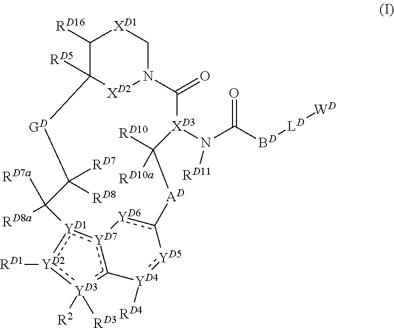

or a pharmaceutically acceptable salt thereof, wherein
the dotted lines represent zero, one, two, three, or four non-adjacent double bonds;
A^{D} is —N(H or CH₃)C(O)—(CH₂)— where the amino nitrogen is bound to the carbon atom of —C(R^{D10a})(R^{D10})—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B^{D} is —CH(R^{D9})— or >C═CR^{D9}R^{D9′} where the carbon is bound to the carbonyl carbon of —N(R^{D11})C(O)—, optionally substituted 3 to 6-membered cycloalkylene, optionally substituted 3 to 6-membered heterocycloalkylene, optionally substituted 6-membered arylene, or 5 to 6-membered heteroarylene;
G^{D} is optionally substituted C₁-C₄ alkylene, optionally substituted C₁-C₄ alkenylene, optionally substituted C₁-C₄ heteroalkylene, —C(O)O—CH(R^{D6})— where —CH(R^{D6})— is bound to —C(R^{D7}R^{D8})—, —C(O)NH—CH(R^{D6})— where —CH(R^{D6})— is bound to_C(R^{D7}R^{D8})—, optionally substituted C₁-C₄ heteroalkylene, or 3 to 8-membered heteroarylene;
L^{D} is absent or a drug linker;
W^{D} is hydrogen, cyano, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, optionally substituted 6 to 10-membered aryl, or optionally substituted 3 to 8-membered heteroaryl;
X^{D1} is optionally substituted C₁-C₂ alkylene, NR^{D}, O, or S(O)_{nD};
X^{D2} is O or NH;
X^{D3} is N or CH;
nD is 0, 1, or 2;
R^{D} is hydrogen, cyano, optionally substituted C₁-C₄ alkyl, optionally substituted C₂-C₄ alkenyl, optionally substituted C₂-C₄ alkynyl, C(O)R^{D′}, C(O)OR^{D′}, C(O)N(R^{D′})₂, S(O)R^{D′}, S(O)₂R^{D′}, or S(O)₂N(R^{D′)}₂; each R^{D′} is, independently, H or optionally substituted C₁-C₄ alkyl;
Y^{D1} is C, CH, or N;
Y^{D2}, Y^{D3}, Y^{D4} and Y^{D7} are, independently, C or N;
Y^{D5} is CH, CH₂, or N;
Y^{D6} is C(O), CH, CH₂, or N;
R^{D1} is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R^{D1} and R^{D2} combine with the atoms to which they are attached to form an optionally substituted 3 to 14-membered heterocycloalkyl;
R^{D2} is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R^{D3} is absent or R^{D2} and R^{D3} combine with the atom to which they are attached to form an optionally substituted 3 to 8-membered cycloalkyl or optionally substituted 3 to 14-membered heterocycloalkyl;
R^{D4} is absent, hydrogen, halogen, cyano, or methyl optionally substituted with 1 to 3 halogens;
R^{D5} is hydrogen, C₁-C₄ alkyl optionally substituted with halogen, cyano, hydroxy, or C₁-C₄ alkoxy, cyclopropyl, or cyclobutyl;
R^{D6} is hydrogen or methyl;
R^{D7} is hydrogen, halogen, or optionally substituted C₁-C₃ alkyl, or
R^{D6} and R^{D7} combine with the carbon atoms to which they are attached to form an optionally substituted 3 to 6-membered cycloalkyl or optionally substituted 3 to 7-membered heterocycloalkyl;
R^{D8} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{D7} and R^{D8} combine with the carbon atom to which they are attached to form C═CR^{D7′}R^{D8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{D7a} and R^{D8a} are, independently, hydrogen, halo, optionally substituted C₁-C₃ alkyl, or combine with the carbon to which they are attached to form a carbonyl;
R^{D7′} is hydrogen, halogen, or optionally substituted C₁-C₃ alkyl;
R^{D8′} is hydrogen, halogen, hydroxyl, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{D7′} and R^{D8′} combine with the carbon atom to which they are attached to form optionally substituted 3 to 6-membered cycloalkyl or optionally substituted 3 to 7-membered heterocycloalkyl;
R^{D9} is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R^{D9} and L^{D} combine with the atoms to which they are attached to form an optionally substituted 3 to 14-membered heterocycloalkyl;
R^{D9′} is hydrogen or optionally substituted C₁-C₆ alkyl;
R^{D10} is hydrogen, halo, hydroxyl, C₁-C₃ alkoxyl, or C₁-C₃ alkyl;
R^{D10a} is hydrogen or halogen;
R^{D11} is hydrogen or C₁-C₃ alkyl; and
R^{D16} is hydrogen or C₁-C₃ alkyl.

In some embodiments, the panRAS inhibitor (D) comprises a compound of Formula (Ic):

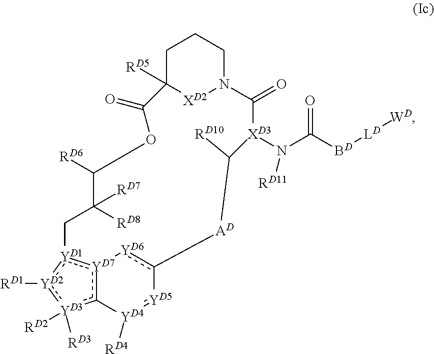

or
a pharmaceutically acceptable salt thereof, wherein
the dotted lines represent zero, one, two, three, or four non-adjacent double bonds;
A^{D} is —N(H or CH₃)C(O)—(CH₂)— where the amino nitrogen is bound to the carbon atom of —CH(R^{D10})—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B^{D} is —CH(R^{D9})— where the carbon is bound to the carbonyl carbon of —N(R^{D11})C(O)—, optionally substituted 3 to 6-membered cycloalkylene, optionally substituted 3 to 6-membered heterocycloalkylene, optionally substituted 6-membered arylene, or 5 to 6-membered heteroarylene;
L^{D} is absent or a drug linker;
W^{D} is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
X^{D2} is O or NH;
X^{D3} is N or CH;
R^{D} is hydrogen, cyano, optionally substituted C₁-C₄ alkyl, optionally substituted C₂-C₄ alkenyl, optionally substituted C₂-C₄ alkynyl, C(O)R^{D′}, C(O)OR^{D′}, C(O)N(R^{D′})₂, S(O)R^{D′}, S(O)₂R^{D′}, or S(O)₂N(R^{D′})₂;
each R^{D′} is, independently, H or optionally substituted C₁-C₄ alkyl;
Y^{D1} is C, CH, or N;
Y^{D2}, Y^{D3}, Y^{D4}, and Y^{D7} are, independently, C or N;
Y^{D5} and Y^{D6} are, independently, CH or N;
R^{D1} is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R^{D2} is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R^{D3} is absent; or
R^{D2} and R^{D3} combine with the atom to which they are attached to form an optionally substituted 3 to 8-membered cycloalkyl or optionally substituted 3 to 14-membered heterocycloalkyl;
R^{D4} is absent, hydrogen, halogen, cyano, or methyl optionally substituted with 1 to 3 halogens;
R^{D5} is hydrogen, C₁-C₄ alkyl optionally substituted with halogen, cyano, hydroxy, or C₁-C₄ alkoxy, cyclopropyl, or cyclobutyl;
R^{D6} is hydrogen or methyl;
R^{D7} is hydrogen, halogen, or optionally substituted 01-O₃ alkyl, or
R^{D6} and R^{D7} combine with the carbon atoms to which they are attached to form an optionally substituted 3 to 6-membered cycloalkyl or optionally substituted 3 to 7-membered heterocycloalkyl;
R^{D8} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{D7} and R^{D8} combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{D7′} is hydrogen, halogen, or optionally substituted C₁-C₃ alkyl;
R^{D8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{D7′} and R^{D8′} combine with the carbon atom to which they are attached to form optionally substituted 3 to 6-membered cycloalkyl or optionally substituted 3 to 7-membered heterocycloalkyl;
R^{D9} is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R^{D10} is hydrogen, hydroxy, C₁-C₃ alkoxy, or C₁-C₃ alkyl; and
R^{D11} is hydrogen or C₁-C₃ alkyl.

In some embodiments, the panRAS inhibitor (D) comprises a compound of Formula (If):

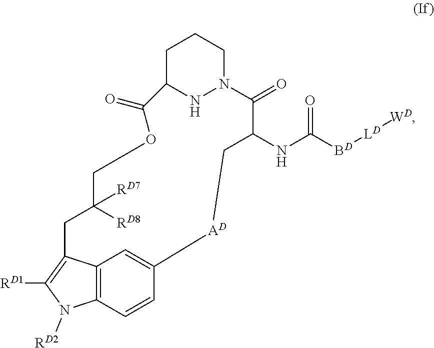

or a pharmaceutically acceptable salt thereof, wherein
A^{D} is —N(H or CH₃)C(O)—(CH₂)— where the amino nitrogen is bound to the carbon atom of —CH₂—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B^{D} is —CH(R^{D9})— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L^{D} is absent or a drug linker;
W^{D} is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R^{D1} is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R^{D2} is C₁-C₆ alkyl or 3 to 6-membered cycloalkyl;
R^{D7} is₁-C₃ alkyl;
R^{D8} is C₁-C₃ alkyl; and
R^{D9} is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

In some embodiments, for any one of embodiments described above R^{D1} is 5 to 10-membered heteroaryl.

In some embodiments, R^{D1} is optionally substituted 6-membered aryl or optionally substituted 6-membered heteroaryl.

In some embodiments, the panRAS inhibitor D is attached to the conjugate linker represented by L at A^{D} or R^{D1} position.

In some embodiments, the panRAS inhibitor D comprises a compound of formula (Ig):

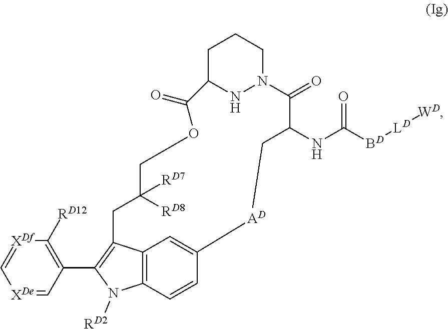

or
a pharmaceutically acceptable salt thereof, wherein:
A^{D} is, optionally substituted 3 to 6-membered cycloalkylene, optionally substituted 3 to 6-membered heterocycloalkylene, optionally substituted 6-membered arylene, or optionally substituted 5 to 6-membered heteroarylene;
B^{D} is —CH(R^{D9})— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L^{D} is absent or a drug linker;
W^{D} is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R^{D2} is C₁-C₆ alkyl or 3 to 6-membered cycloalkyl;
R^{D7} is C₁-C₃ alkyl;
R^{D8} is C₁-C₃ alkyl;
R^{D9} is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
X^{De} is N, CH, or CR^{D17};
X^{Df} is N or CH;
R^{D12} is optionally substituted C₁-C₆ alkyl or optionally substituted C₁-C₆ heteroalkyl; and
R^{D17} is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

In some embodiments, A^{D} is optionally substituted 6-membered arylene.

In some embodiments, A^{D} is optionally substituted 5 to 6-membered heteroarylene.

In some embodiments, B^{D} is —CHR^{D9}_.

In some embodiments, R^{D9} is optionally substituted C₁-C₆ alkyl or optionally substituted 3 to 6-membered cycloalkyl.

In some embodiments, the drug linker in the panRAS inhibitors described in any one of the embodiments above is the structure of Formula II:

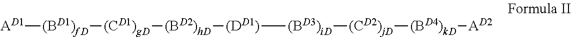

wherein
A^{D1} is a bond between the drug linker and B; A^{D2} is a bond between Wand the drug linker;
B^{D1}, B^{D2}, B^{D3}, and B^{D4} each, independently, is selected from optionally substituted C₁-C₂ alkylene, optionally substituted C₁-C₃ heteroalkylene, O, S, and NR^{DN}; R^{DN} is hydrogen, optionally substituted C₁-C₄ alkyl, optionally substituted C₁-C₃ cycloalkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl;
C^{D1} and C^{D2} are each, independently, selected from carbonyl, thiocarbonyl, sulphonyl, or phosphoryl;
fD, gD, hD, iD, jD, and kD are each, independently, 0 or 1; and
D^{D1} is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A^{D1}-(B^{D1})_{fD}—(C^{D1})_{gD}—(B^{D2})_{hD}— to —(B^{D3})_{iD}—(CD²)_{Dj}-(B^{D4})_{Dk}-A^{D2}.

In some embodiments, the drug linker has the structure of Formula Ila:

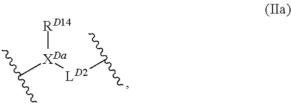

wherein
X^{Da} is absent or N;
R^{D14} is absent, hydrogen, optionally substituted C₁-C₆ alkyl, or optionally substituted C₁-C₃ cycloalkyl; and
L^{D2} is absent, —C(O)—, —SO₂—, optionally substituted C₁-C₄ alkylene or optionally substituted C₁-C₄ heteroalkylene, wherein at least one of X^{Da}, R^{D14}, or L^{D2} is present.

In some embodiments, for the panRAS inhibitors described in any one of the embodiments above, W^{D} is hydrogen.

In some embodiments, for the panRAS inhibitors described in any one of the embodiments above, W^{D} is C₀-C₄ alkyl optionally substituted 3 to 11-membered heterocycloalkyl.

In some embodiments, the panRAS inhibitor D is attached to the conjugate linker represented by L at A^{D} or R^{D17} position.

In some embodiments, the panRAS inhibitor D comprises a compound of formula (Ih):

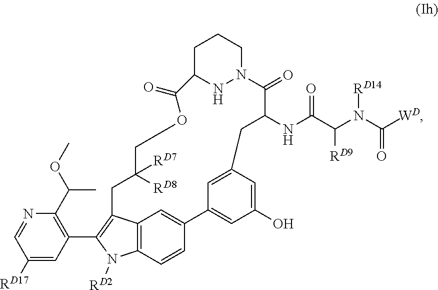

a pharmaceutically acceptable salt thereof, wherein:
R^{D2} is C₁-C₃ alkyl;
R^{D7} is C₁-C₃ alkyl;
R^{D8} is C₁₋₃ alkyl;
R^{D9} is C₁-C₆ alkyl;
R^{D14} is hydrogen or C₁-C₆ alkyl,
R^{D17} is optionally substituted 3 to 6-membered cycloalkyl or optionally substituted 3 to 6-membered heterocycloalkyl; and
W^{D} is optionally substituted 3 to 11-membered heterocycloalkyl.

In some embodiments, R^{D9} is C₁-C₃ alkyl; R^{D14} is C₁-C₃ alkyl; R^{D17} is optionally substituted 3 to 6-membered heterocycloalkyl; and W^{D} is optionally substituted 5 to 6-membered heterocycloalkyl.

In some embodiments, the panRAS inhibitor D comprises a compound represented by

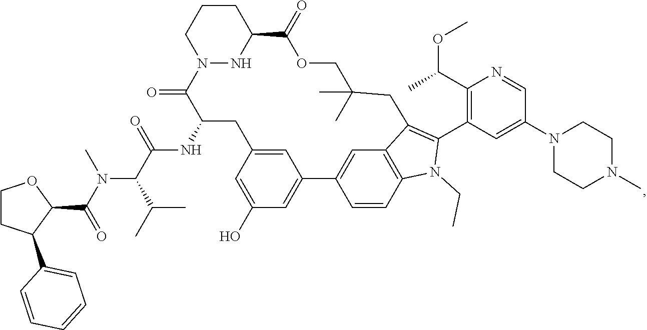 [SMILES: CCn1c(-c2cc(N3CCN(C)CC3)cnc2[C@H](C)OC)c2c3cc(ccc31)-c1cc(O)cc(c1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2]

or
a pharmaceutically acceptable salt thereof.

In some embodiments, the panRAS inhibitor D comprises a compound of formula (Ij):

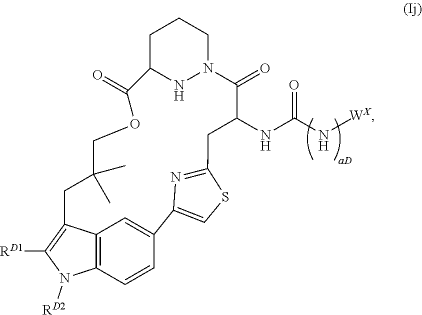

or
a pharmaceutically acceptable salt thereof, wherein aD is 0 or 1. The definitions of the other variables are provided in any one of the embodiments described above.

In some embodiments, the panRAS inhibitor D comprises a compound of formula (Ik):

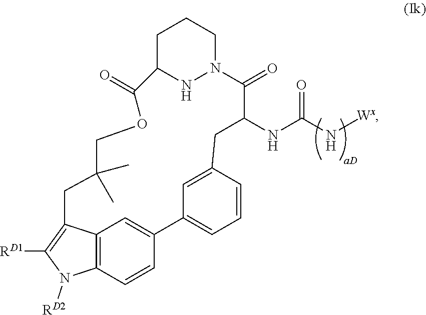

a pharmaceutically acceptable salt thereof, wherein aD is 0 or 1. The definitions of the other variables are provided in any one of the embodiments described above.

In some embodiments, the panRAS inhibitor D comprises a compound of formula (Im):

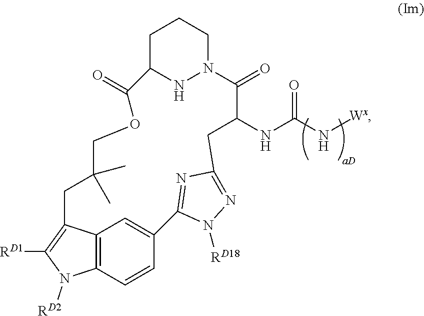

or
a pharmaceutically acceptable salt thereof, wherein aD is 0 or 1. The definitions of the other variables are provided in any one of the embodiments described above.

In some embodiments, the panRAS inhibitor D comprises a compound of formula (In):

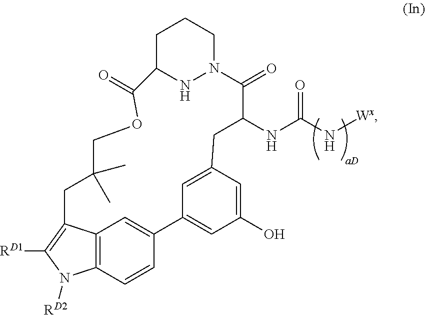

a pharmaceutically acceptable salt thereof, wherein aD is 0 or 1. The definitions of the other variables are provided in any one of the embodiments described above.

In some embodiments, D represents a panRAS inhibitor attached to the conjugate linker L by a covalent bond, wherein the panRAS inhibitor is selected from a compound in Table A1:

TABLE A1

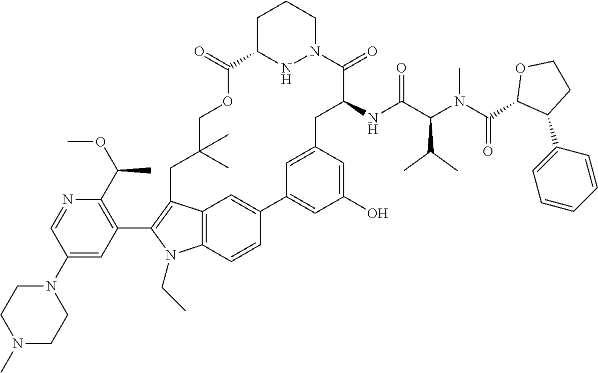 [SMILES: CCn1c(-c2cc(N3CCN(C)CC3)cnc2[C@H](C)OC)c2c3cc(ccc31)-c1cc(O)cc(c1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2]

Compound D101

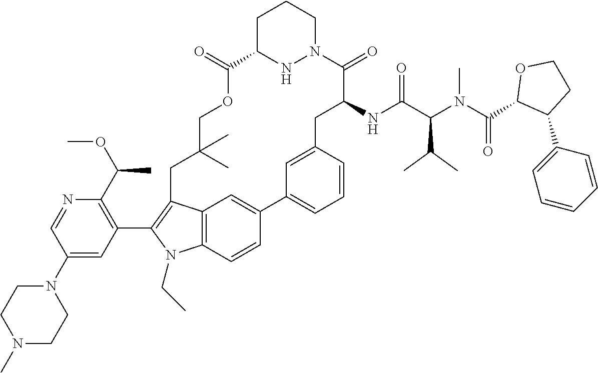 [SMILES: CCn1c(-c2cc(N3CCN(C)CC3)cnc2[C@H](C)OC)c2c3cc(ccc31)-c1cccc(c1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2]

Compound D102

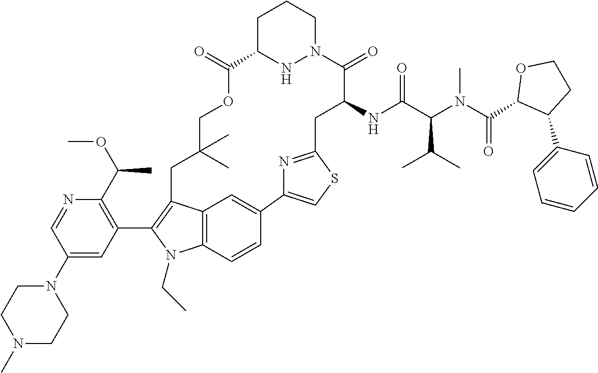 [SMILES: CCn1c(-c2cc(N3CCN(C)CC3)cnc2[C@H](C)OC)c2c3cc(ccc31)-c1csc(n1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2]

Compound D103

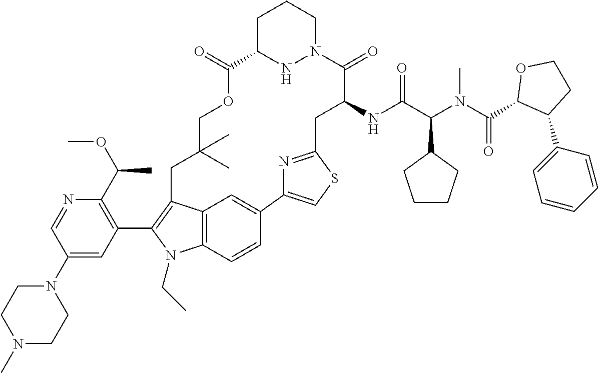 [SMILES: CCn1c(-c2cc(N3CCN(C)CC3)cnc2[C@H](C)OC)c2c3cc(ccc31)-c1csc(n1)C[C@H](NC(=O)[C@H](C1CCCC1)N(C)C(=O)[C@@H]1OCC[C@@H]1c1ccccc1)C(=O)N1CCC[C@H](N1)C(=O)OCC(C)(C)C2]

Compound D104

or a pharmaceutically acceptable salt thereof.

In some embodiments, the panRAS inhibitor 0 comprises a formula selected from any one of the formulae in Table A2, or a pharmaceutically acceptable salt thereof.

TABLE A2

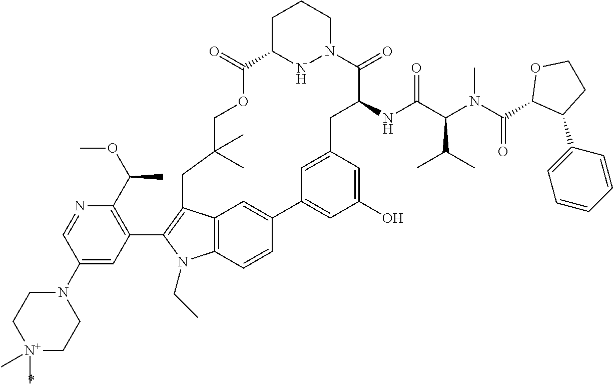 [SMILES: CCn1c(-c2cc(N3CC[N+](C)(C)CC3)cnc2[C@H](C)OC)c2c3cc(ccc31)-c1cc(O)cc(c1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2]

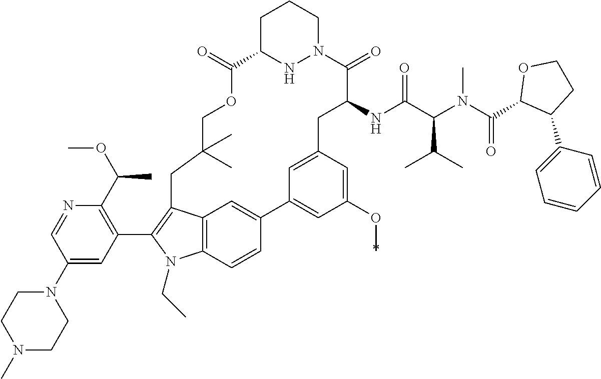 [SMILES: CCn1c(-c2cc(N3CCN(C)CC3)cnc2[C@H](C)OC)c2c3cc(ccc31)-c1cc(cc(OC)c1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2]

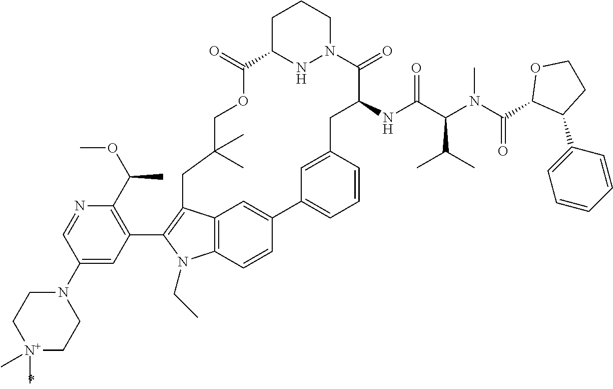 [SMILES: CCn1c(-c2cc(N3CC[N+](C)(C)CC3)cnc2[C@H](C)OC)c2c3cc(ccc31)-c1cccc(c1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2]

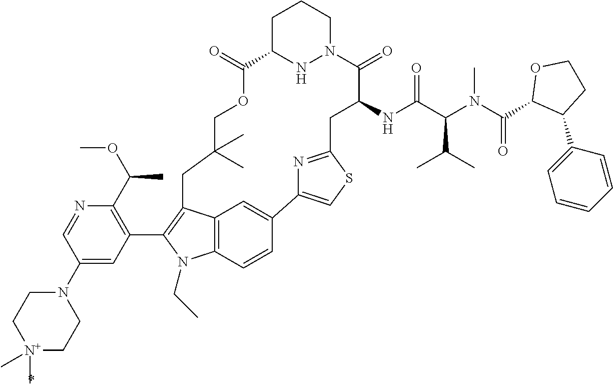 [SMILES: CCn1c(-c2cc(N3CC[N+](C)(C)CC3)cnc2[C@H](C)OC)c2c3cc(ccc31)-c1csc(n1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2]

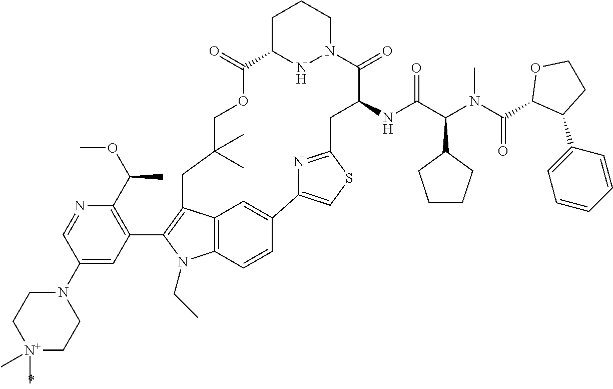 [SMILES: CCn1c(-c2cc(N3CC[N+](C)(C)CC3)cnc2[C@H](C)OC)c2c3cc(ccc31)-c1csc(n1)C[C@H](NC(=O)[C@H](C1CCCC1)N(C)C(=O)[C@@H]1OCC[C@@H]1c1ccccc1)C(=O)N1CCC[C@H](N1)C(=O)OCC(C)(C)C2]

wherein — represents a bond to the conjugate linker.

In some embodiments, -(L-D) is formed from a compound selected from Table B or an enantiomer, a diastereoisomer, and/or a pharmaceutically acceptable salt thereof. In some embodiments, the maleimide group

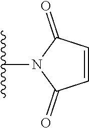 [SMILES: *N1C(=O)C=CC1=O]

in the compound of Table B form a covalent bond with the antibody or antigen-binding fragment thereof (Ab) to form the ADC compound of formula (1) comprising a

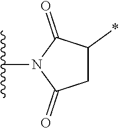 [SMILES: *C1CC(=O)N(*)C1=O]

moiety, wherein * indicates the connection point to Ab. For compounds in Table A1, Table A2, Table B and Table 1, depending on their electronic charge, these compounds can contain one pharmaceutically acceptable monovalent anionic counterion M₁⁻. In some embodiments, the monovalent anionic counterion M₁⁻ can be selected from bromide, chloride, iodide, acetate, trifluoroacetate, benzoate, mesylate, tosylate, triflate, formate, or the like. In some embodiments, the monovalent anionic counterion M₁⁻ is trifluoroacetate or formate.

TABLE B
Exemplary Linker-Drug Groups

| Name | Linker Payload Structure |
|---|---|
| L101- D101 | 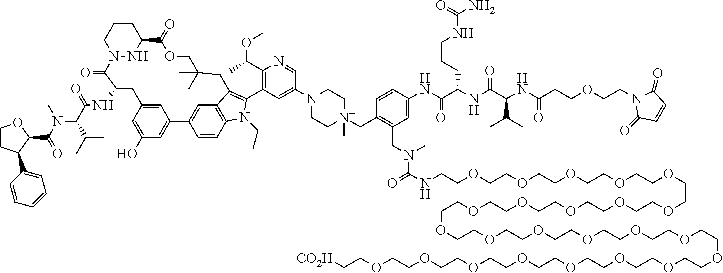 [SMILES: CCn1c(-c2cc(N3CC[N+](C)(Cc4ccc(NC(=O)[C@H](CCCNC(N)=O)NC(=O)[C@@H](NC(=O)CCOCCN5C(=O)C=CC5=O)C(C)C)cc4CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)CC3)cnc2[C@H](C)OC)c2c3cc(ccc31)-c1cc(O)cc(c1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2] |
| L102- D101 | 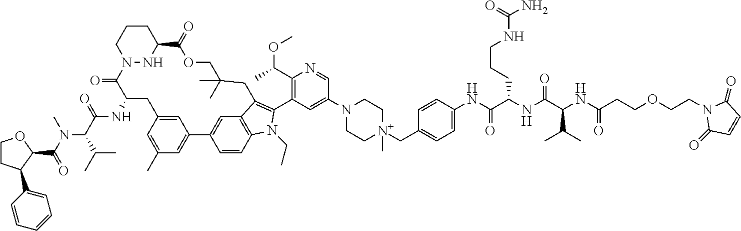 [SMILES: CCn1c(-c2cc(N3CC[N+](C)(Cc4ccc(NC(=O)[C@H](CCCNC(N)=O)NC(=O)[C@@H](NC(=O)CCOCCN5C(=O)C=CC5=O)C(C)C)cc4)CC3)cnc2[C@H](C)OC)c2c3cc(ccc31)-c1cc(C)cc(c1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2] |
| L103- D101 | 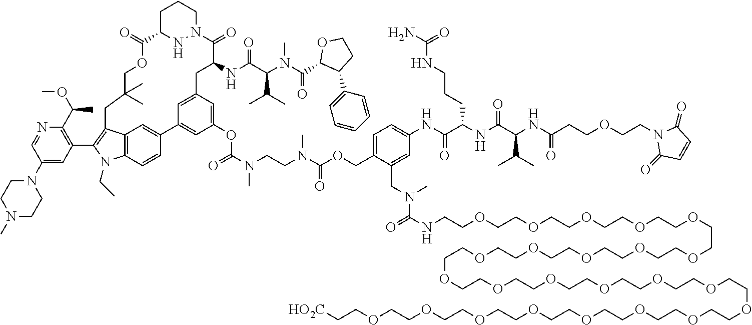 [SMILES: CCn1c(-c2cc(N3CCN(C)CC3)cnc2[C@H](C)OC)c2c3cc(ccc31)-c1cc(cc(OC(=O)N(C)CCN(C)C(=O)OCc3ccc(NC(=O)[C@H](CCCNC(N)=O)NC(=O)[C@@H](NC(=O)CCOCCN4C(=O)C=CC4=O)C(C)C)cc3CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)c1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2] |
| L101- D102 | 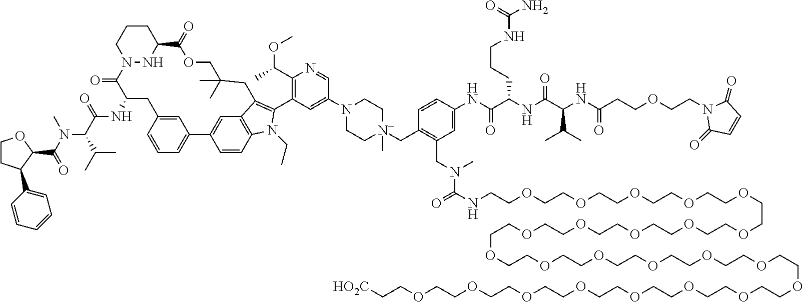 [SMILES: CCn1c(-c2cc(N3CC[N+](C)(Cc4ccc(NC(=O)[C@H](CCCNC(N)=O)NC(=O)[C@@H](NC(=O)CCOCCN5C(=O)C=CC5=O)C(C)C)cc4CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)CC3)cnc2[C@H](C)OC)c2c3cc(ccc31)-c1cccc(c1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2] |
| L101- D103 | 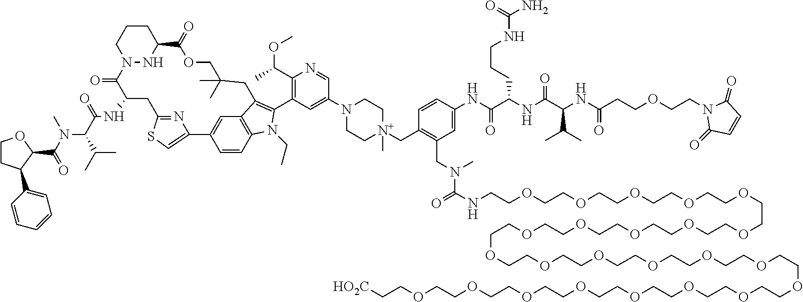 [SMILES: CCn1c(-c2cc(N3CC[N+](C)(Cc4ccc(NC(=O)[C@H](CCCNC(N)=O)NC(=O)[C@@H](NC(=O)CCOCCN5C(=O)C=CC5=O)C(C)C)cc4CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)CC3)cnc2[C@H](C)OC)c2c3cc(ccc31)-c1csc(n1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2] |
| L101- D104 | 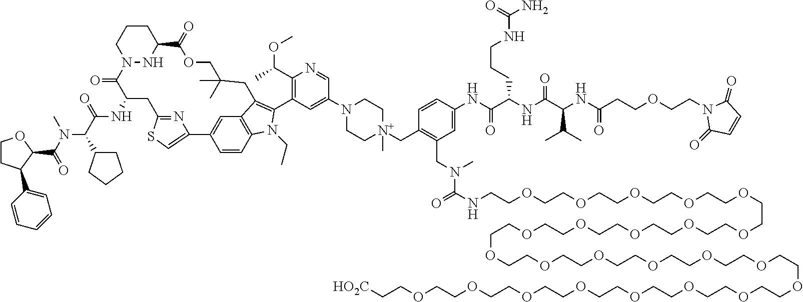 [SMILES: CCn1c(-c2cc(N3CC[N+](C)(Cc4ccc(NC(=O)[C@H](CCCNC(N)=O)NC(=O)[C@@H](NC(=O)CCOCCN5C(=O)C=CC5=O)C(C)C)cc4CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)CC3)cnc2[C@H](C)OC)c2c3cc(ccc31)-c1csc(n1)C[C@H](NC(=O)[C@H](C1CCCC1)N(C)C(=O)[C@@H]1OCC[C@@H]1c1ccccc1)C(=O)N1CCC[C@H](N1)C(=O)OCC(C)(C)C2] |
| L102- D102 | 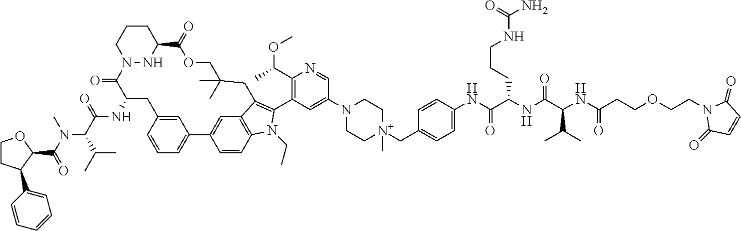 [SMILES: CCn1c(-c2cc(N3CC[N+](C)(Cc4ccc(NC(=O)[C@H](CCCNC(N)=O)NC(=O)[C@@H](NC(=O)CCOCCN5C(=O)C=CC5=O)C(C)C)cc4)CC3)cnc2[C@H](C)OC)c2c3cc(ccc31)-c1cccc(c1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2] |
| L102- D103 | 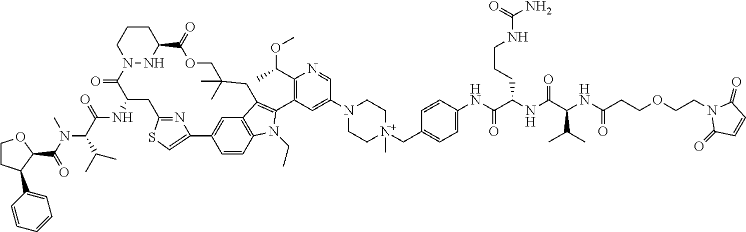 [SMILES: CCn1c(-c2cc(N3CC[N+](C)(Cc4ccc(NC(=O)[C@H](CCCNC(N)=O)NC(=O)[C@@H](NC(=O)CCOCCN5C(=O)C=CC5=O)C(C)C)cc4)CC3)cnc2[C@H](C)OC)c2c3cc(ccc31)-c1csc(n1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2] |
| L102- D104 | 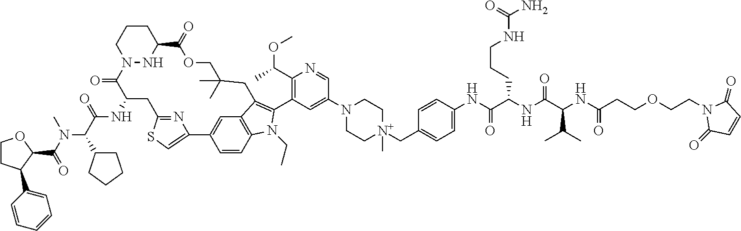 [SMILES: CCn1c(-c2cc(N3CC[N+](C)(Cc4ccc(NC(=O)[C@H](CCCNC(N)=O)NC(=O)[C@@H](NC(=O)CCOCCN5C(=O)C=CC5=O)C(C)C)cc4)CC3)cnc2[C@H](C)OC)c2c3cc(ccc31)-c1csc(n1)C[C@H](NC(=O)[C@H](C1CCCC1)N(C)C(=O)[C@@H]1OCC[C@@H]1c1ccccc1)C(=O)N1CCC[C@H](N1)C(=O)OCC(C)(C)C2] |

In some embodiments, the antibody-drug conjugate has a formula according to any one of the structures shown in Table 1.

TABLE 1
ADC Structures

| Name* | ADC Structure* |
|---|---|
| Ab-L101- D101 | 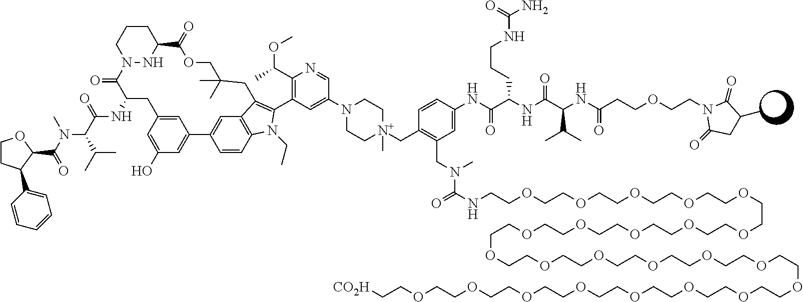 [SMILES: CCn1c(-c2cc(N3CC[N+](C)(Cc4ccc(NC(=O)[C@H](CCCNC(N)=O)NC(=O)[C@@H](NC(=O)CCOCCN5C(=O)CC(C)C5=O)C(C)C)cc4CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)CC3)cnc2[C@H](C)OC)c2c3cc(ccc31)-c1cc(O)cc(c1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2] |
| Ab-L102- D101 | 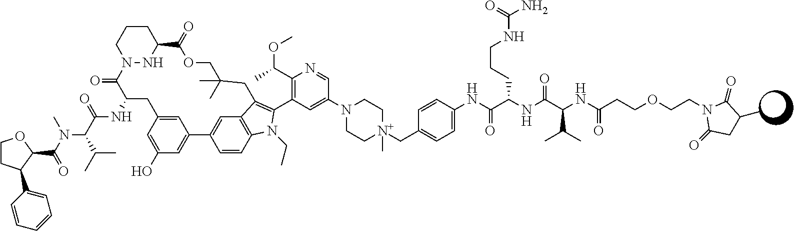 [SMILES: CCn1c(-c2cc(N3CC[N+](C)(Cc4ccc(NC(=O)[C@H](CCCNC(N)=O)NC(=O)[C@@H](NC(=O)CCOCCN5C(=O)CC(C)C5=O)C(C)C)cc4)CC3)cnc2[C@H](C)OC)c2c3cc(ccc31)-c1cc(O)cc(c1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2] |
| Ab-L103- D101 | 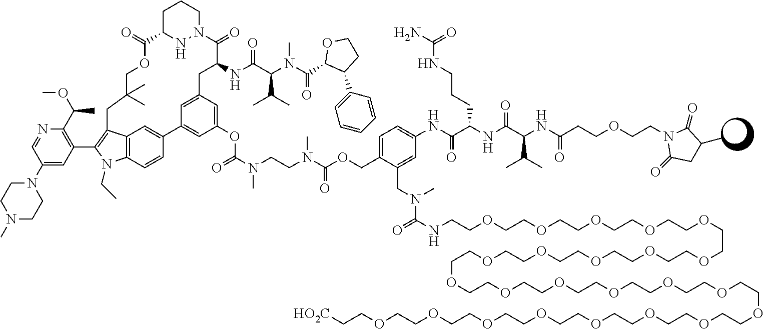 [SMILES: CCn1c(-c2cc(N3CCN(C)CC3)cnc2[C@H](C)OC)c2c3cc(ccc31)-c1cc(cc(OC(=O)N(C)CCN(C)C(=O)OCc3ccc(NC(=O)[C@H](CCCNC(N)=O)NC(=O)[C@@H](NC(=O)CCOCCN4C(=O)CC(C)C4=O)C(C)C)cc3CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)c1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2] |
| Ab-L101- D102 | 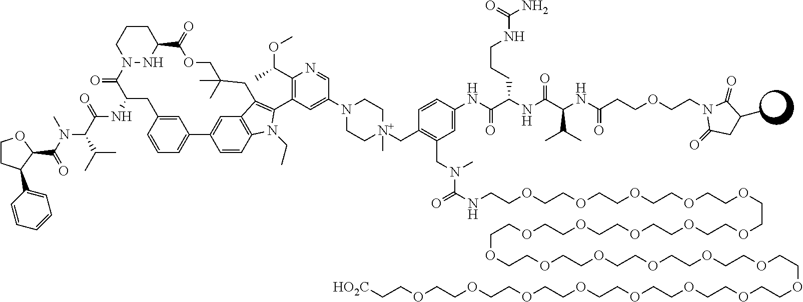 [SMILES: CCn1c(-c2cc(N3CC[N+](C)(Cc4ccc(NC(=O)[C@H](CCCNC(N)=O)NC(=O)[C@@H](NC(=O)CCOCCN5C(=O)CC(C)C5=O)C(C)C)cc4CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)CC3)cnc2[C@H](C)OC)c2c3cc(ccc31)-c1cccc(c1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2] |
| Ab-L101- D103 | 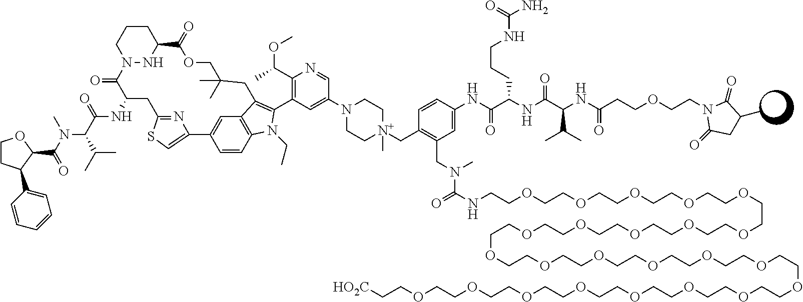 [SMILES: CCn1c(-c2cc(N3CC[N+](C)(Cc4ccc(NC(=O)[C@H](CCCNC(N)=O)NC(=O)[C@@H](NC(=O)CCOCCN5C(=O)CC(C)C5=O)C(C)C)cc4CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)CC3)cnc2[C@H](C)OC)c2c3cc(ccc31)-c1csc(n1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2] |
| Ab-L101- D104 | 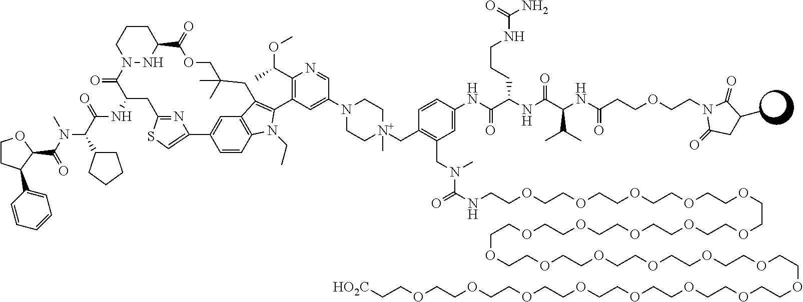 [SMILES: CCn1c(-c2cc(N3CC[N+](C)(Cc4ccc(NC(=O)[C@H](CCCNC(N)=O)NC(=O)[C@@H](NC(=O)CCOCCN5C(=O)CC(C)C5=O)C(C)C)cc4CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)CC3)cnc2[C@H](C)OC)c2c3cc(ccc31)-c1csc(n1)C[C@H](NC(=O)[C@H](C1CCCC1)N(C)C(=O)[C@@H]1OCC[C@@H]1c1ccccc1)C(=O)N1CCC[C@H](N1)C(=O)OCC(C)(C)C2] |
| Ab-L102- D102 | 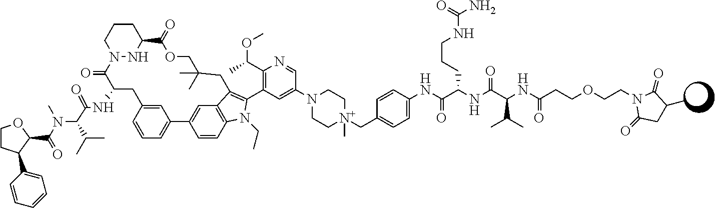 [SMILES: CCn1c(-c2cc(N3CC[N+](C)(Cc4ccc(NC(=O)[C@H](CCCNC(N)=O)NC(=O)[C@@H](NC(=O)CCOCCN5C(=O)CC(C)C5=O)C(C)C)cc4)CC3)cnc2[C@H](C)OC)c2c3cc(ccc31)-c1cccc(c1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2] |
| Ab-L102- D103 | 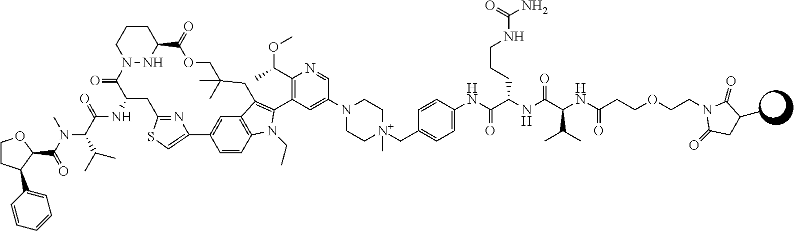 [SMILES: CCn1c(-c2cc(N3CC[N+](C)(Cc4ccc(NC(=O)[C@H](CCCNC(N)=O)NC(=O)[C@@H](NC(=O)CCOCCN5C(=O)CC(C)C5=O)C(C)C)cc4)CC3)cnc2[C@H](C)OC)c2c3cc(ccc31)-c1csc(n1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2] |
| Ab-L102- D104 | 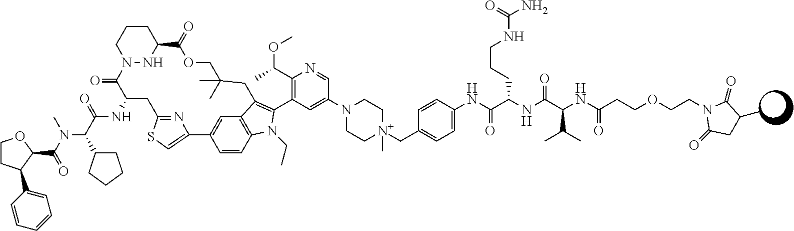 [SMILES: CCn1c(-c2cc(N3CC[N+](C)(Cc4ccc(NC(=O)[C@H](CCCNC(N)=O)NC(=O)[C@@H](NC(=O)CCOCCN5C(=O)CC(C)C5=O)C(C)C)cc4)CC3)cnc2[C@H](C)OC)c2c3cc(ccc31)-c1csc(n1)C[C@H](NC(=O)[C@H](C1CCCC1)N(C)C(=O)[C@@H]1OCC[C@@H]1c1ccccc1)C(=O)N1CCC[C@H](N1)C(=O)OCC(C)(C)C2] |

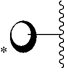

or Ab: Any antibody or antigen-binding fragment thereof described herein, e.g., anti-EphA2 antibody, anti-B7-H3 antibody, or antigen-binding fragments thereof.

The ADCs depicted above can also be represented by the following formula:

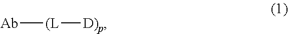

wherein Ab or

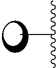

represents an antibody or an antigen-binding fragment thereof covalently linked to the linker-payload (L-D) depicted above; p is an integer from 1 to 16. In some embodiments, p is an integer from 1 to 8. In some embodiments, p is an integer from 1 to 5. In some embodiments, p is an integer from 2 to 4. In some embodiments, p is 1. In some embodiments, p is 2. In some embodiments, p is 4. In some embodiments, p is determined by liquid chromatography-mass spectrometry (LC-MS).

As used herein, “L-D” refers to the linker-payloads, linker-drugs, or linker-compounds disclosed herein and the terms “L #-D #” is used to refer to a specific linker-drug disclosed herein, while the codes “D #” is used to refer to a specific compound unless otherwise specified, including an enantiomer, diastereoisomer, atropisomer, deuterated derivative, and/or pharmaceutically acceptable salt of any of the foregoing.

In some embodiments, for ADCs depicted in Table 1, Ab is an antibody or an antigen-binding fragment thereof described herein. In some embodiments, for ADCs depicted in Table 1, Ab is an anti-EphA2 antibody or antigen-binding fragment thereof. In some embodiments, Ab is an anti-B7-H3 antibody or antigen-binding fragment thereof.

In some embodiments, the antibody or antigen-binding fragment binds to a target antigen on a cancer cell. In some embodiments, the target antigen is EphA2 or B7-H3 (CD276).

In some embodiments, the target antigen is EphA2. In some embodiments, the target antigen is B7-H3 (CD276).

In some embodiments, the antibody or antigen-binding fragment is an anti-EphA2 antibody or antigen-binding fragment. In some embodiments, the anti-EphA2 antibody or antigen-binding fragment comprises three heavy chain complementarity determining regions (HCDRs) and three light chain complementarity determining regions (LCDRs) selected from the group consisting of:
1) heavy chain CDR1 (HCDR1) consisting of SEQ ID NO:17, heavy chain CDR2 (HCDR2) consisting of SEQ ID NO:18, heavy chain CDR3 (HCDR3) consisting of SEQ ID NO:19; light chain CDR1 (LCDR1) consisting of SEQ ID NO:26, light chain CDR2 (LCDR2) consisting of SEQ ID NO:27, and light chain CDR3 (LCDR3) consisting of SEQ ID NO:28;
2) heavy chain CDR1 (HCDR1) consisting of SEQ ID NO:20, heavy chain CDR2 (HCDR2) consisting of SEQ ID NO:21, heavy chain CDR3 (HCDR3) consisting of SEQ ID NO:19; light chain CDR1 (LCDR1) consisting of SEQ ID NO:29, light chain CDR2 (LCDR2) consisting of SEQ ID NO:30, and light chain CDR3 (LCDR3) consisting of SEQ ID NO:31;
3) heavy chain CDR1 (HCDR1) consisting of SEQ ID NO:22, heavy chain CDR2 (HCDR2) consisting of SEQ ID NO:23, heavy chain CDR3 (HCDR3) consisting of SEQ ID NO:24; light chain CDR1 (LCDR1) consisting of SEQ ID NO:32, light chain CDR2 (LCDR2) consisting of SEQ ID NO:27, and light chain CDR3 (LCDR3) consisting of SEQ ID NO:31; and
4) heavy chain CDR1 (HCDR1) consisting of SEQ ID NO:25, heavy chain CDR2 (HCDR2) consisting of SEQ ID NO:21, heavy chain CDR3 (HCDR3) consisting of SEQ ID NO:19; light chain CDR1 (LCDR1) consisting of SEQ ID NO:29, light chain CDR2 (LCDR2) consisting of SEQ ID NO:30, and light chain CDR3 (LCDR3) consisting of SEQ ID NO:31.

In some embodiments, the anti-EphA2 antibody or antigen-binding fragment comprises a heavy chain variable region comprising an amino acid sequence of SEQ ID NO:11, and a light chain variable region comprising an amino acid sequence of SEQ ID NO:12. In some embodiments, the anti-EphA2 antibody or antigen-binding fragment comprises an IgG1 heavy chain constant domain or a modified IgG1 heavy chain constant domain. In some embodiments, the IgG1 heavy chain constant domain comprises a cysteine residue (C) at position 152 and position 375. In some embodiments, the antibody or antigen-binding fragment comprises an Ig kappa light chain constant domain.

In some embodiments, the anti-EphA2 antibody or antigen-binding fragment comprises a heavy chain comprising an amino acid sequence of SEQ ID NO:3, and a light chain comprising an amino acid sequence of SEQ ID NO:5.

In some embodiments, the antibody or antigen-binding fragment is an anti-B7-H3 (CD276) antibody or antigen-binding fragment. In some embodiments, the anti-B7-H3 (CD276) antibody or antigen-binding fragment comprises three heavy chain complementarity determining regions (HCDRs) and three light chain complementarity determining regions (LCDRs) selected from the group consisting of:
1) heavy chain CDR1 (HCDR1) consisting of SEQ ID NO:33, heavy chain CDR2 (HCDR2) consisting of SEQ ID NO:34, heavy chain CDR3 (HCDR3) consisting of SEQ ID NO:35; light chain CDR1 (LCDR1) consisting of SEQ ID NO:42, light chain CDR2 (LCDR2) consisting of SEQ ID NO:43, and light chain CDR3 (LCDR3) consisting of SEQ ID NO:44;
2) heavy chain CDR1 (HCDR1) consisting of SEQ ID NO:36, heavy chain CDR2 (HCDR2) consisting of SEQ ID NO:37, heavy chain CDR3 (HCDR3) consisting of SEQ ID NO:35; light chain CDR1 (LCDR1) consisting of SEQ ID NO:45, light chain CDR2 (LCDR2) consisting of SEQ ID NO:46, and light chain CDR3 (LCDR3) consisting of SEQ ID NO:47;
3) heavy chain CDR1 (HCDR1) consisting of SEQ ID NO:38, heavy chain CDR2 (HCDR2) consisting of SEQ ID NO:39, heavy chain CDR3 (HCDR3) consisting of SEQ ID NO:40; light chain CDR1 (LCDR1) consisting of SEQ ID NO:48, light chain CDR2 (LCDR2) consisting of SEQ ID NO:43, and light chain CDR3 (LCDR3) consisting of SEQ ID NO:47;
4) heavy chain CDR1 (HCDR1) consisting of SEQ ID NO:41, heavy chain CDR2 (HCDR2) consisting of SEQ ID NO:37, heavy chain CDR3 (HCDR3) consisting of SEQ ID NO:35; light chain CDR1 (LCDR1) consisting of SEQ ID NO:45, light chain CDR2 (LCDR2) consisting of SEQ ID NO:46, and light chain CDR3 (LCDR3) consisting of SEQ ID NO:47;
5) heavy chain CDR1 (HCDR1) consisting of SEQ ID NO:49, heavy chain CDR2 (HCDR2) consisting of SEQ ID NO:50, heavy chain CDR3 (HCDR3) consisting of SEQ ID NO:51; light chain CDR1 (LCDR1) consisting of SEQ ID NO:58, light chain CDR2 (LCDR2) consisting of SEQ ID NO:59, and light chain CDR3 (LCDR3) consisting of SEQ ID NO:60;
6) heavy chain CDR1 (HCDR1) consisting of SEQ ID NO:52, heavy chain CDR2 (HCDR2) consisting of SEQ ID NO:53, heavy chain CDR3 (HCDR3) consisting of SEQ ID NO:51; light chain CDR1 (LCDR1) consisting of SEQ ID NO:61 light chain CDR2 (LCDR2) consisting of SEQ ID NO:62, and light chain CDR3 (LCDR3) consisting of SEQ ID NO:63;
7) heavy chain CDR1 (HCDR1) consisting of SEQ ID NO:54, heavy chain CDR2 (HCDR2) consisting of SEQ ID NO:55, heavy chain CDR3 (HCDR3) consisting of SEQ ID NO:56; light chain CDR1 (LCDR1) consisting of SEQ ID NO:58, light chain CDR2 (LCDR2) consisting of SEQ ID NO:59, and light chain CDR3 (LCDR3) consisting of SEQ ID NO:63; and
8) heavy chain CDR1 (HCDR1) consisting of SEQ ID NO:57, heavy chain CDR2 (HCDR2) consisting of SEQ ID NO:53, heavy chain CDR3 (HCDR3) consisting of SEQ ID NO:51; light chain CDR1 (LCDR1) consisting of SEQ ID NO:61, light chain CDR2 (LCDR2) consisting of SEQ ID NO:62, and light chain CDR3 (LCDR3) consisting of SEQ ID NO:63.

In some embodiments, the anti-B7-H3 (CD276) antibody or antigen-binding fragment comprises a heavy chain variable region comprising an amino acid sequence of SEQ ID NO:13, and a light chain variable region comprising an amino acid sequence of SEQ ID NO:14. In some embodiments, the antibody or antigen-binding fragment comprises an IgG1 heavy chain constant domain or a modified IgG1 heavy chain constant domain. In some embodiments, the IgG1 heavy chain constant domain comprises a cysteine residue (C) at position 152 and position 375. In some embodiments, the antibody or antigen-binding fragment comprises an Ig kappa light chain constant domain.

In some embodiments, the anti-B7-H3 (CD276) antibody or antigen-binding fragment comprises a heavy chain variable region comprising an amino acid sequence of SEQ ID NO:15, and a light chain variable region comprising an amino acid sequence of SEQ ID NO:16. In some embodiments, the antibody or antigen-binding fragment comprises an IgG1 heavy chain constant domain or a modified IgG1 heavy chain constant domain. In some embodiments, the IgG1 heavy chain constant domain comprises a cysteine residue (C) at position 152 and position 375. In some embodiments, the antibody or antigen-binding fragment comprises an Ig kappa light chain constant domain.

In some embodiments, the anti-B7-H3 (CD276) antibody or antigen-binding fragment comprises a heavy chain comprising an amino acid sequence of SEQ ID NO:7, and a light chain comprising an amino acid sequence of SEQ ID NO:8.

In some embodiments, the anti-B7-H3 (CD276) antibody or antigen-binding fragment comprises a heavy chain comprising an amino acid sequence of SEQ ID NO:9, and a light chain comprising an amino acid sequence of SEQ ID NO:10.

Also provided herein, in some embodiments, are compositions comprising multiple copies of an antibody-drug conjugate (e.g., any of the exemplary antibody-drug conjugates described herein). In some embodiments, the average p of the antibody-drug conjugates in the composition is from about 2 to about 4.

Also provided herein, in some embodiments, are pharmaceutical compositions comprising an antibody-drug conjugate (e.g., any of the exemplary antibody-drug conjugates described herein) or a composition (e.g., any of the exemplary compositions described herein), and a pharmaceutically acceptable carrier.

Further provided herein, in some embodiments, are therapeutic uses for the described ADC compounds and compositions, e.g., in treating a cancer. In some embodiments, the present disclosure provides methods of treating a cancer (e.g., a cancer that expresses an antigen targeted by the antibody or antigen-binding fragment of the ADC, such as EphA2 or B7-H3 (CD276)). In some embodiments, the present disclosure provides methods of reducing or slowing the expansion of a cancer cell population in a subject. In some embodiments, the present disclosure provides methods of determining whether a subject having or suspected of having a cancer will be responsive to treatment with an ADC compound or composition disclosed herein.

An exemplary embodiment is a method of treating a subject having or suspected of having a cancer, comprising administering to the subject a therapeutically effective amount of an antibody-drug conjugate, composition, or pharmaceutical composition (e.g., any of the exemplary antibody-drug conjugates, compositions, or pharmaceutical compositions disclosed herein). In some embodiments, the cancer expresses a target antigen. In some embodiments, the target antigen is BCMA, CD33, HER2, CD38, CD48, CD79b, PCAD, CD74, CD138, SLAMF7, CD123, CLL1, FLT3, CD7, CKIT, CD56, DLL3, DLK1, B7-H3, B7-H4, EGFR, CD71, EPCAM, FOLR1, ENPP3, MET, AXL, SLC34A2 (NaPi2b), Nectin4, TROP2, LIV1, CD46, MSLN, CD142 (F3), MUC1, MUC16, SLC39A6, TFRC, TACSTD2, GPNMB, EphA2, CD56, SEZ6, CD25, CCR8, CEACAM5, CEACAM6, 4-1 BB, 5AC, 5T4, Alpha-fetoprotein, angiopoietin 2, ASLG659, TCLI, BMPRIB, Brevican BCAN, BEHAB, C242 antigen, C5, CA-125, CA-125 (imitation), CA-IX (Carbonic anhydrase 9), CCR4, CD140a, CD152, CD19, CD20, CD200, CD21 (C3DR) I), CD22 (B-cell receptor CD22-B isoform), CD221, CD23 (gE receptor), CD28, CD30 (TNFRSF8), CD37, CD4, CD40, CD44 v6, CD51, CD52, CD70, CD72 (Lyb-2, B-cell differentiation antigen CD72), CD79a, CD80, CD166 (ALCAM), CDH17, CA9, CEA, CEA-related antigen, ch4D5, CLDN18.2, CRIPTO (CR, CRI, CRGF, TDGF1, CFC1B), CTLA-4, CXCR5, DLL4, DR5, E16 (LATI, SLC7A5), EGFL7, EphB2R (DRT, ERK, Hek5, EPHT3, Tyro5), Episialin, ERBB3, ETBR (Endothelin type B receptor), FCRHI (Fc receptor-like protein I), FcRH2 (IFGP4, IRTA4, SPAPI, SPAP IB, SPAP IC), Fibronectin extra domain-B, Frizzled receptor, GD2, GD3 ganglioside, GEDA, HER1, HER2/neu, HER3, HGF, HLA-DOB, HLA-DR, Human scatter factor receptor kinase, IGF-I receptor, IL-13, IL20R (ZCYTOR7), IL-6, ILGF2, ILFRIR, integrin u, IRTA2 (Immunoglobulin superfamily receptor translocation associated 2), Lewis-Y antigen, LY64 (RP105), LY6E, STEAP1, ADAM9, PTK7, MMP14, TM4SF1, ITGB6, FXYD5, MCP-I, MDP (DPEPI), MPF, MSLN, SMR, mesothelin, megakaryocyte, PD-I, PDCDI, PDGF-R u, Prostate specific membrane antigen (PSMA), PSCA (Prostate stem cell antigen precursor), PRLR (Prolactin Receptor), PSCA hlg, RANKL, RON, SDCI, Sema Sb, STEAP I, STEAP2, PCANAP I, STAMP I, STEAP2, STMP, prostate cancer associated gene I, TAG-72, TEMI, Tenascin C, TENB2, (TMEFF2, tomoregulin, TPEF, HPPI, TR), TGF-IJ, TRAIL-E2, TRAIL-RI, TRAIL-R2, T17M4 (BR22450, FLJ20041, TRPM4, TRPM4B, transient receptor potential cation channel subfamily M, member 4), TWEAK-R, TYRP I (glycoprotein 75), VEGF, VEGF-A, EGFR-I, VEGFR-2, or Vimentin.

In some embodiments, the target antigen is EphA2 or B7-H3 (CD276).

In some embodiments, the target antigen is EphA2.

In some embodiments, the target antigen is B7-H3 (CD276).

In some embodiments, the cancer is a tumor or a hematological cancer. In some embodiments, the cancer is a breast cancer including ER positive breast cancer, multiple myeloma, plasma cell myeloma, leukemia, lymphoma, sarcoma, gastric or stomach cancer, acute myeloid leukemia, bladder cancer, brain cancer, bone marrow cancer, cervical cancer, chronic lymphocytic leukemia, colorectal cancer, pancreatic cancer, esophageal cancer, hepatocellular cancer, lymphoblastic leukemia including acute lymphoblastic leukemia, follicular lymphoma, lymphoid malignancies of T-cell or B-cell origin, metastatic castration resistant prostate cancer, bladder urothelial carcinoma, melanoma, myelogenous leukemia, myeloma, oral cancer, ovarian cancer, non-small cell lung cancer, prostate cancer, small cell lung cancer, spleen cancer, or head and neck cancer.

Another exemplary embodiment is a method of reducing or inhibiting the growth of a tumor in a subject, comprising administering to the subject a therapeutically effective amount of an antibody-drug conjugate, composition, or pharmaceutical composition (e.g., any of the exemplary antibody-drug conjugates, compositions, or pharmaceutical compositions disclosed herein). In some embodiments, the tumor expresses a target antigen. In some embodiments, the target antigen is BCMA, CD33, HER2, CD38, CD48, CD79b, PCAD, CD74, CD138, SLAMF7, CD123, CLL1, FLT3, CD7, CKIT, CD56, DLL3, DLK1, B7-H3, B7-H4, EGFR, CD71, EPCAM, FOLR1, ENPP3, MET, AXL, SLC34A2 (NaPi2b), Nectin4, TROP2, LIV1, CD46, MSLN, CD142 (F3), MUC1, MUC16, SLC39A6, TFRC, TACSTD2, GPNMB, EphA2, CD56, SEZ6, CD25, CCR8, CEACAM5, CEACAM6, 4-1 BB, 5AC, 5T4, Alpha-fetoprotein, angiopoietin 2, ASLG659, TCLI, BMPRIB, Brevican BCAN, BEHAB, C242 antigen, C5, CA-125, CA-125 (imitation), CA-IX (Carbonic anhydrase 9), CCR4, CD140a, CD152, CD19, CD20, CD200, CD21 (C3DR) I), CD22 (B-cell receptor CD22-B isoform), CD221, CD23 (gE receptor), CD28, CD30 (TNFRSF8), CD37, CD4, CD40, CD44 v6, CD51, CD52, CD70, CD72 (Lyb-2, B-cell differentiation antigen CD72), CD79a, CD80, CD166 (ALCAM), CDH17, CA9, CEA, CEA-related antigen, ch4D5, CLDN18.2, CRIPTO (CR, CRI, CRGF, TDGF1, CFC11B), CTLA-4, CXCR5, DLL4, DR5, E16 (LATI, SLC7A5), EGFL7, EphB2R (DRT, ERK, Hek5, EPHT3, Tyro5), Episialin, ERBB3, ETBR (Endothelin type B receptor), FCRHI (Fc receptor-like protein I), FcRH2 (IFGP4, IRTA4, SPAPI, SPAP IB, SPAP IC), Fibronectin extra domain-B, Frizzled receptor, GD2, GD3 ganglioside, GEDA, HER1, HER2/neu, HER3, HGF, HLA-DOB, HLA-DR, Human scatter factor receptor kinase, IGF-I receptor, IL-13, IL20R (ZCYTOR7), IL-6, ILGF2, ILFRIR, integrin u, IRTA2 (Immunoglobulin superfamily receptor translocation associated 2), Lewis-Y antigen, LY64 (RP105), LY6E, STEAP1, ADAM9, PTK7, MMP14, TM4SF1, ITGB6, FXYD5, MCP-I, MDP (DPEPI), MPF, MSLN, SMR, mesothelin, megakaryocyte, PD-I, PDCDI, PDGF-R u, Prostate specific membrane antigen (PSMA), PSCA (Prostate stem cell antigen precursor), PRLR (Prolactin Receptor), PSCA hlg, RANKL, RON, SDCI, Sema Sb, STEAP I, STEAP2, PCANAP I, STAMP I, STEAP2, STMP, prostate cancer associated gene I, TAG-72, TEMI, Tenascin C, TENB2, (TMEFF2, tomoregulin, TPEF, HPPI, TR), TGF-IJ, TRAIL-E2, TRAIL-RI, TRAIL-R2, T17M4 (BR22450, FLJ20041, TRPM4, TRPM4B, transient receptor potential cation channel subfamily M, member 4), TWEAK-R, TYRP I (glycoprotein 75), VEGF, VEGF-A, EGFR-I, VEGFR-2, or Vimentin. In some embodiments, the target antigen is EphA2 or B7-H3 (CD276). In some embodiments, the target antigen is EphA2. In some embodiments, the target antigen is B7-H3 (CD276). In some embodiments, the tumor is a breast cancer including ER positive breast cancer, multiple myeloma, plasma cell myeloma, leukemia, lymphoma, sarcoma, gastric or stomach cancer, acute myeloid leukemia, bladder cancer, brain cancer, bone marrow cancer, cervical cancer, chronic lymphocytic leukemia, colorectal cancer, pancreatic cancer, esophageal cancer, hepatocellular cancer, lymphoblastic leukemia including acute lymphoblastic leukemia, follicular lymphoma, lymphoid malignancies of T-cell or B-cell origin, metastatic castration resistant prostate cancer, bladder urothelial carcinoma, melanoma, myelogenous leukemia, myeloma, oral cancer, ovarian cancer, non-small cell lung cancer, prostate cancer, small cell lung cancer, spleen cancer, or head and neck cancer. In some embodiments, administration of the antibody-drug conjugate, composition, or pharmaceutical composition reduces or inhibits the growth of the tumor by at least about 10%, at least about 20%, at least about 30%, at least about 40%, at least about 50%, at least about 60%, at least about 70%, at least about 80%, at least about 90%, at least about 95%, or at least about 99%.

Another exemplary embodiment is a method of reducing or slowing the expansion of a cancer cell population in a subject, comprising administering to the subject a therapeutically effective amount of an antibody-drug conjugate, composition, or pharmaceutical composition (e.g., any of the exemplary antibody-drug conjugates, compositions, or pharmaceutical compositions disclosed herein). In some embodiments, the cancer cell population expresses a target antigen. In some embodiments, the target antigen is BCMA, CD33, HER2, CD38, CD48, CD79b, PCAD, CD74, CD138, SLAMF7, CD123, CLL1, FLT3, CD7, CKIT, CD56, DLL3, DLK1, B7-H3, B7-H4, EGFR, CD71, EPCAM, FOLR1, ENPP3, MET, AXL, SLC34A2 (NaPi2b), Nectin4, TROP2, LIV1, CD46, MSLN, CD142 (F3), MUC1, MUC16, SLC39A6, TFRC, TACSTD2, GPNMB, EphA2, CD56, SEZ6, CD25, CCR8, CEACAM5, CEACAM6, 4-1BB, 5AC, 5T4, Alpha-fetoprotein, angiopoietin 2, ASLG659, TCLI, BMPRIB, Brevican BCAN, BEHAB, C242 antigen, C5, CA-125, CA-125 (imitation), CA-IX (Carbonic anhydrase 9), CCR4, CD140a, CD152, CD19, CD20, CD200, CD21 (C3DR) I), CD22 (B-cell receptor CD22-B isoform), CD221, CD23 (gE receptor), CD28, CD30 (TNFRSF8), CD37, CD4, CD40, CD44 v6, CD51, CD52, CD70, CD72 (Lyb-2, B-cell differentiation antigen CD72), CD79a, CD80, CD166 (ALCAM), CDH17, CA9, CEA, CEA-related antigen, ch4D5, CLDN18.2, CRIPTO (CR, CRI, CRGF, TDGF1, CFC1B), CTLA-4, CXCR5, DLL4, DR5, E16 (LATI, SLC7A5), EGFL7, EphB2R (DRT, ERK, Hek5, EPHT3, Tyro5), Episialin, ERBB3, ETBR (Endothelin type B receptor), FCRHI (Fc receptor-like protein I), FcRH2 (IFGP4, IRTA4, SPAPI, SPAP IB, SPAP IC), Fibronectin extra domain-B, Frizzled receptor, GD2, GD3 ganglioside, GEDA, HER1, HER2/neu, HER3, HGF, HLA-DOB, HLA-DR, Human scatter factor receptor kinase, IGF-I receptor, IL-13, IL20R (ZCYTOR7), IL-6, ILGF2, ILFRIR, integrin u, IRTA2 (Immunoglobulin superfamily receptor translocation associated 2), Lewis-Y antigen, LY64 (RP105), LY6E, STEAP1, ADAM9, PTK7, MMP14, TM4SF1, ITGB6, FXYD5, MCP-I, MDP (DPEPI), MPF, MSLN, SMR, mesothelin, megakaryocyte, PD-I, PDCDI, PDGF-R u, Prostate specific membrane antigen (PSMA), PSCA (Prostate stem cell antigen precursor), PRLR (Prolactin Receptor), PSCA hlg, RANKL, RON, SDCI, Sema Sb, STEAP I, STEAP2, PCANAP I, STAMP I, STEAP2, STMP, prostate cancer associated gene I, TAG-72, TEMI, Tenascin C, TENB2, (TMEFF2, tomoregulin, TPEF, HPPI, TR), TGF-IJ, TRAIL-E2, TRAIL-RI, TRAIL-R2, T17M4 (BR22450, FLJ20041, TRPM4, TRPM4B, transient receptor potential cation channel subfamily M, member 4), TWEAK-R, TYRP I (glycoprotein 75), VEGF, VEGF-A, EGFR-I, VEGFR-2, or Vimentin. In some embodiments, the target antigen is EphA2 or B7-H3 (CD276). In some embodiments, the target antigen is EphA2. In some embodiments, the target antigen is B7-H3 (CD276). In some embodiments, the cancer cell population is from a tumor or a hematological cancer. In some embodiments, the cancer cell population is a breast cancer including ER positive breast cancer, multiple myeloma, plasma cell myeloma, leukemia, lymphoma, sarcoma, gastric or stomach cancer, acute myeloid leukemia, bladder cancer, brain cancer, bone marrow cancer, cervical cancer, chronic lymphocytic leukemia, colorectal cancer, pancreatic cancer, esophageal cancer, hepatocellular cancer, lymphoblastic leukemia including acute lymphoblastic leukemia, follicular lymphoma, lymphoid malignancies of T-cell or B-cell origin, metastatic castration resistant prostate cancer, bladder urothelial carcinoma, melanoma, myelogenous leukemia, myeloma, oral cancer, ovarian cancer, non-small cell lung cancer, prostate cancer, small cell lung cancer, spleen cancer, or head and neck cancer.

In some embodiments, administration of the antibody-drug conjugate, composition, or pharmaceutical composition reduces the cancer cell population by at least about 10%, at least about 20%, at least about 30%, at least about 40%, at least about 50%, at least about 60%, at least about 70%, at least about 80%, at least about 90%, at least about 95%, or at least about 99%. In some embodiments, administration of the antibody-drug conjugate, composition, or pharmaceutical composition slows the expansion of the cancer cell population by at least about 10%, at least about 20%, at least about 30%, at least about 40%, at least about 50%, at least about 60%, at least about 70%, at least about 80%, at least about 90%, at least about 95%, or at least about 99%.

Another exemplary embodiment is an antibody-drug conjugate, composition, or pharmaceutical composition (e.g., any of the exemplary antibody-drug conjugates, compositions, or pharmaceutical compositions disclosed herein) for use in treating a subject having or suspected of having a cancer. In some embodiments, the cancer expresses a target antigen. In some embodiments, the target antigen is BCMA, CD33, HER2, CD38, CD48, CD79b, PCAD, CD74, CD138, SLAMF7, CD123, CLL1, FLT3, CD7, CKIT, CD56, DLL3, DLK1, B7-H3, B7-H4, EGFR, CD71, EPCAM, FOLR1, ENPP3, MET, AXL, SLC34A2 (NaPi2b), Nectin4, TROP2, LIV1, CD46, MSLN, CD142 (F3), MUC1, MUC16, SLC39A6, TFRC, TACSTD2, GPNMB, EphA2, CD56, SEZ6, CD25, CCR8, CEACAM5, CEACAM6, 4-1BB, 5AC, 5T4, Alpha-fetoprotein, angiopoietin 2, ASLG659, TCLI, BMPRIB, Brevican BCAN, BEHAB, C242 antigen, C5, CA-125, CA-125 (imitation), CA-IX (Carbonic anhydrase 9), CCR4, CD140a, CD152, CD19, CD20, CD200, CD21 (C3DR) I), CD22 (B-cell receptor CD22-B isoform), CD221, CD23 (gE receptor), CD28, CD30 (TNFRSF8), CD37, CD4, CD40, CD44 v6, CD51, CD52, CD70, CD72 (Lyb-2, B-cell differentiation antigen CD72), CD79a, CD80, CD166 (ALCAM), CDH17, CA9, CEA, CEA-related antigen, ch4D5, CLDN18.2, CRIPTO (CR, CRI, CRGF, TDGF1, CFC1B), CTLA-4, CXCR5, DLL4, DR5, E16 (LATI, SLC7A5), EGFL7, EphB2R (DRT, ERK, Hek5, EPHT3, Tyro5), Episialin, ERBB3, ETBR (Endothelin type B receptor), FCRHI (Fc receptor-like protein I), FcRH2 (IFGP4, IRTA4, SPAPI, SPAP IB, SPAP IC), Fibronectin extra domain-B, Frizzled receptor, GD2, GD3 ganglioside, GEDA, HER1, HER2/neu, HER3, HGF, HLA-DOB, HLA-DR, Human scatter factor receptor kinase, IGF-I receptor, IL-13, IL20R (ZCYTOR7), IL-6, ILGF2, ILFRIR, integrin u, IRTA2 (Immunoglobulin superfamily receptor translocation associated 2), Lewis-Y antigen, LY64 (RP105), LY6E, STEAP1, ADAM9, PTK7, MMP14, TM4SF1, ITGB6, FXYD5, MCP-I, MDP (DPEPI), MPF, MSLN, SMR, mesothelin, megakaryocyte, PD-I, PDCDI, PDGF-R u, Prostate specific membrane antigen (PSMA), PSCA (Prostate stem cell antigen precursor), PRLR (Prolactin Receptor), PSCA hlg, RANKL, RON, SDCI, Sema Sb, STEAP I, STEAP2, PCANAP I, STAMP I, STEAP2, STMP, prostate cancer associated gene I, TAG-72, TEMI, Tenascin C, TENB2, (TMEFF2, tomoregulin, TPEF, HPPI, TR), TGF-IJ, TRAIL-E2, TRAIL-RI, TRAIL-R2, T17M4 (BR22450, FLJ20041, TRPM4, TRPM4B, transient receptor potential cation channel subfamily M, member 4), TWEAK-R, TYRP I (glycoprotein 75), VEGF, VEGF-A, EGFR-I, VEGFR-2, or Vimentin. In some embodiments, the target antigen is EphA2 or B7-H3 (CD276). In some embodiments, the target antigen is EphA2. In some embodiments, the target antigen is B7-H3 (CD276). In some embodiments, the cancer is a tumor or a hematological cancer. In some embodiments, the cancer is a breast cancer including ER positive breast cancer, multiple myeloma, plasma cell myeloma, leukemia, lymphoma, sarcoma, gastric or stomach cancer, acute myeloid leukemia, bladder cancer, brain cancer, bone marrow cancer, cervical cancer, chronic lymphocytic leukemia, colorectal cancer, pancreatic cancer, esophageal cancer, hepatocellular cancer, lymphoblastic leukemia including acute lymphoblastic leukemia, follicular lymphoma, lymphoid malignancies of T-cell or B-cell origin, metastatic castration resistant prostate cancer, bladder urothelial carcinoma, melanoma, myelogenous leukemia, myeloma, oral cancer, ovarian cancer, non-small cell lung cancer, prostate cancer, small cell lung cancer, spleen cancer, or head and neck cancer.

Another exemplary embodiment is a use of an antibody-drug conjugate, composition, or pharmaceutical composition (e.g., any of the exemplary antibody-drug conjugates, compositions, or pharmaceutical compositions disclosed herein) in treating a subject having or suspected of having a cancer. In some embodiments, the cancer expresses a target antigen. In some embodiments, the target antigen is BCMA, CD33, HER2, CD38, CD48, CD79b, PCAD, CD74, CD138, SLAMF7, CD123, CLL1, FLT3, CD7, CKIT, CD56, DLL3, DLK1, B7-H3, B7-H4, EGFR, CD71, EPCAM, FOLR1, ENPP3, MET, AXL, SLC34A2 (NaPi2b), Nectin4, TROP2, LIV1, CD46, MSLN, CD142 (F3), MUC1, MUC16, SLC39A6, TFRC, TACSTD2, GPNMB, EphA2, CD56, SEZ6, CD25, CCR8, CEACAM5, CEACAM6, 4-1BB, 5AC, 5T4, Alpha-fetoprotein, angiopoietin 2, ASLG659, TCLI, BMPRIB, Brevican BCAN, BEHAB, C242 antigen, C5, CA-125, CA-125 (imitation), CA-IX (Carbonic anhydrase 9), CCR4, CD140a, CD152, CD19, CD20, CD200, CD21 (C3DR) I), CD22 (B-cell receptor CD22-B isoform), CD221, CD23 (gE receptor), CD28, CD30 (TNFRSF8), CD37, CD4, CD40, CD44 v6, CD51, CD52, CD70, CD72 (Lyb-2, B-cell differentiation antigen CD72), CD79a, CD80, CD166 (ALCAM), CDH17, CA9, CEA, CEA-related antigen, ch4D5, CLDN18.2, CRIPTO (CR, CRI, CRGF, TDGF1, CFC1B), CTLA-4, CXCR5, DLL4, DR5, E16 (LATI, SLC7A5), EGFL7, EphB2R (DRT, ERK, Hek5, EPHT3, Tyro5), Episialin, ERBB3, ETBR (Endothelin type B receptor), FCRHI (Fc receptor-like protein I), FcRH2 (IFGP4, IRTA4, SPAPI, SPAP IB, SPAP IC), Fibronectin extra domain-B, Frizzled receptor, GD2, GD3 ganglioside, GEDA, HER1, HER2/neu, HER3, HGF, HLA-DOB, HLA-DR, Human scatter factor receptor kinase, IGF-I receptor, IL-13, IL20R (ZCYTOR7), IL-6, ILGF2, ILFRIR, integrin u, IRTA2 (Immunoglobulin superfamily receptor translocation associated 2), Lewis-Y antigen, LY64 (RP105), LY6E, STEAP1, ADAM9, PTK7, MMP14, TM4SF1, ITGB6, FXYD5, MCP-I, MDP (DPEPI), MPF, MSLN, SMR, mesothelin, megakaryocyte, PD-I, PDCDI, PDGF-R u, Prostate specific membrane antigen (PSMA), PSCA (Prostate stem cell antigen precursor), PRLR (Prolactin Receptor), PSCA hlg, RANKL, RON, SDCI, Sema Sb, STEAP I, STEAP2, PCANAP I, STAMP I, STEAP2, STMP, prostate cancer associated gene I, TAG-72, TEMI, Tenascin C, TENB2, (TMEFF2, tomoregulin, TPEF, HPPI, TR), TGF-IJ, TRAIL-E2, TRAIL-RI, TRAIL-R2, T17M4 (BR22450, FLJ20041, TRPM4, TRPM4B, transient receptor potential cation channel subfamily M, member 4), TWEAK-R, TYRP I (glycoprotein 75), VEGF, VEGF-A, EGFR-I, VEGFR-2, or Vimentin. In some embodiments, the target antigen is EphA2 or B7-H3 (CD276). In some embodiments, the target antigen is EphA2. In some embodiments, the target antigen is B7-H3 (CD276). In some embodiments, the cancer is a tumor or a hematological cancer. In some embodiments, the cancer is a breast cancer including ER positive breast cancer, multiple myeloma, plasma cell myeloma, leukemia, lymphoma, sarcoma, gastric or stomach cancer, acute myeloid leukemia, bladder cancer, brain cancer, bone marrow cancer, cervical cancer, chronic lymphocytic leukemia, colorectal cancer, pancreatic cancer, esophageal cancer, hepatocellular cancer, lymphoblastic leukemia including acute lymphoblastic leukemia, follicular lymphoma, lymphoid malignancies of T-cell or B-cell origin, metastatic castration resistant prostate cancer, bladder urothelial carcinoma, melanoma, myelogenous leukemia, myeloma, oral cancer, ovarian cancer, non-small cell lung cancer, prostate cancer, small cell lung cancer, spleen cancer, or head and neck cancer.

Another exemplary embodiment is a use of an antibody-drug conjugate, composition, or pharmaceutical composition (e.g., any of the exemplary antibody-drug conjugates, compositions, or pharmaceutical compositions disclosed herein) in a method of manufacturing a medicament for treating a subject having or suspected of having a cancer. In some embodiments, the cancer expresses a target antigen. In some embodiments, the target antigen is BCMA, CD33, HER2, CD38, CD48, CD79b, PCAD, CD74, CD138, SLAMF7, CD123, CLL1, FLT3, CD7, CKIT, CD56, DLL3, DLK1, B7-H3, B7-H4, EGFR, CD71, EPCAM, FOLR1, ENPP3, MET, AXL, SLC34A2 (NaPi2b), Nectin4, TROP2, LIV1, CD46, MSLN, CD142 (F3), MUC1, MUC16, SLC39A6, TFRC, TACSTD2, GPNMB, EphA2, CD56, SEZ6, CD25, CCR8, CEACAM5, CEACAM6, 4-1BB, 5AC, 5T4, Alpha-fetoprotein, angiopoietin 2, ASLG659, TCLI, BMPRIB, Brevican BCAN, BEHAB, C242 antigen, C5, CA-125, CA-125 (imitation), CA-IX (Carbonic anhydrase 9), CCR4, CD140a, CD152, CD19, CD20, CD200, CD21 (C3DR) I), CD22 (B-cell receptor CD22-B isoform), CD221, CD23 (gE receptor), CD28, CD30 (TNFRSF8), CD37, CD4, CD40, CD44 v6, CD51, CD52, CD70, CD72 (Lyb-2, B-cell differentiation antigen CD72), CD79a, CD80, CD166 (ALCAM), CDH17, CA9, CEA, CEA-related antigen, ch4D5, CLDN18.2, CRIPTO (CR, CRI, CRGF, TDGF1, CFC1B), CTLA-4, CXCR5, DLL4, DR5, E16 (LATI, SLC7A5), EGFL7, EphB2R (DRT, ERK, Hek5, EPHT3, Tyro5), Episialin, ERBB3, ETBR (Endothelin type B receptor), FCRHI (Fc receptor-like protein I), FcRH2 (IFGP4, IRTA4, SPAPI, SPAP IB, SPAP IC), Fibronectin extra domain-B, Frizzled receptor, GD2, GD3 ganglioside, GEDA, HER1, HER2/neu, HER3, HGF, HLA-DOB, HLA-DR, Human scatter factor receptor kinase, IGF-I receptor, IL-13, IL20R (ZCYTOR7), IL-6, ILGF2, ILFRIR, integrin u, IRTA2 (Immunoglobulin superfamily receptor translocation associated 2), Lewis-Y antigen, LY64 (RP105), LY6E, STEAP1, ADAM9, PTK7, MMP14, TM4SF1, ITGB6, FXYD5, MCP-I, MDP (DPEPI), MPF, MSLN, SMR, mesothelin, megakaryocyte, PD-I, PDCDI, PDGF-R u, Prostate specific membrane antigen (PSMA), PSCA (Prostate stem cell antigen precursor), PRLR (Prolactin Receptor), PSCA hlg, RANKL, RON, SDCI, Sema Sb, STEAP I, STEAP2, PCANAP I, STAMP I, STEAP2, STMP, prostate cancer associated gene I, TAG-72, TEMI, Tenascin C, TENB2, (TMEFF2, tomoregulin, TPEF, HPPI, TR), TGF-IJ, TRAIL-E2, TRAIL-RI, TRAIL-R2, T17M4 (BR22450, FLJ20041, TRPM4, TRPM4B, transient receptor potential cation channel subfamily M, member 4), TWEAK-R, TYRP I (glycoprotein 75), VEGF, VEGF-A, EGFR-I, VEGFR-2, or Vimentin. In some embodiments, the target antigen is EphA2 or B7-H3 (CD276). In some embodiments, the target antigen is EphA2. In some embodiments, the target antigen is B7-H3 (CD276). In some embodiments, the cancer is a tumor or a hematological cancer. In some embodiments, the cancer is a breast cancer including ER positive breast cancer, multiple myeloma, plasma cell myeloma, leukemia, lymphoma, sarcoma, gastric or stomach cancer, acute myeloid leukemia, bladder cancer, brain cancer, bone marrow cancer, cervical cancer, chronic lymphocytic leukemia, colorectal cancer, pancreatic cancer, esophageal cancer, hepatocellular cancer, lymphoblastic leukemia including acute lymphoblastic leukemia, follicular lymphoma, lymphoid malignancies of T-cell or B-cell origin, metastatic castration resistant prostate cancer, bladder urothelial carcinoma, melanoma, myelogenous leukemia, myeloma, oral cancer, ovarian cancer, non-small cell lung cancer, prostate cancer, small cell lung cancer, spleen cancer, or head and neck cancer.

Another exemplary embodiment is a method of determining whether a subject having or suspected of having a cancer will be responsive to treatment with an antibody-drug conjugate, composition, or pharmaceutical composition (e.g., any of the exemplary antibody-drug conjugates, compositions, or pharmaceutical compositions disclosed herein) by providing a biological sample from the subject; contacting the sample with the antibody-drug conjugate; and detecting binding of the antibody-drug conjugate to cancer cells in the sample. In some embodiments, the cancer cells in the sample express a target antigen. In some embodiments, the cancer expresses a target antigen. In some embodiments, the target antigen is BCMA, CD33, HER2, CD38, CD48, CD79b, PCAD, CD74, CD138, SLAMF7, CD123, CLL1, FLT3, CD7, CKIT, CD56, DLL3, DLK1, B7-H3, B7-H4, EGFR, CD71, EPCAM, FOLR1, ENPP3, MET, AXL, SLC34A2 (NaPi2b), Nectin4, TROP2, LIV1, CD46, MSLN, CD142 (F3), MUC1, MUC16, SLC39A6, TFRC, TACSTD2, GPNMB, EphA2, CD56, SEZ6, CD25, CCR8, CEACAM5, CEACAM6, 4-1BB, 5AC, 5T4, Alpha-fetoprotein, angiopoietin 2, ASLG659, TCLI, BMPRIB, Brevican BCAN, BEHAB, C242 antigen, C5, CA-125, CA-125 (imitation), CA-IX (Carbonic anhydrase 9), CCR4, CD140a, CD152, CD19, CD20, CD200, CD21 (C3DR) I), CD22 (B-cell receptor CD22-B isoform), CD221, CD23 (gE receptor), CD28, CD30 (TNFRSF8), CD37, CD4, CD40, CD44 v6, CD51, CD52, CD70, CD72 (Lyb-2, B-cell differentiation antigen CD72), CD79a, CD80, CD166 (ALCAM), CDH17, CA9, CEA, CEA-related antigen, ch4D5, CLDN18.2, CRIPTO (CR, CRI, CRGF, TDGF1, CFC1B), CTLA-4, CXCR5, DLL4, DR5, E16 (LATI, SLC7A5), EGFL7, EphB2R (DRT, ERK, Hek5, EPHT3, Tyro5), Episialin, ERBB3, ETBR (Endothelin type B receptor), FCRHI (Fc receptor-like protein I), FcRH2 (IFGP4, IRTA4, SPAPI, SPAP IB, SPAP IC), Fibronectin extra domain-B, Frizzled receptor, GD2, GD3 ganglioside, GEDA, HER1, HER2/neu, HER3, HGF, HLA-DOB, HLA-DR, Human scatter factor receptor kinase, IGF-I receptor, IL-13, IL20R (ZCYTOR7), IL-6, ILGF2, ILFRIR, integrin u, IRTA2 (Immunoglobulin superfamily receptor translocation associated 2), Lewis-Y antigen, LY64 (RP105), LY6E, STEAP1, ADAM9, PTK7, MMP14, TM4SF1, ITGB6, FXYD5, MCP-I, MDP (DPEPI), MPF, MSLN, SMR, mesothelin, megakaryocyte, PD-1, PDCDI, PDGF-R u, Prostate specific membrane antigen (PSMA), PSCA (Prostate stem cell antigen precursor), PRLR (Prolactin Receptor), PSCA hlg, RANKL, RON, SDCI, Sema Sb, STEAP I, STEAP2, PCANAP I, STAMP 1, STEAP2, STMP, prostate cancer associated gene 1, TAG-72, TEMI, Tenascin C, TENB2, (TMEFF2, tomoregulin, TPEF, HPPI, TR), TGF-IJ, TRAIL-E2, TRAIL-RI, TRAIL-R2, T17M4 (BR22450, FLJ20041, TRPM4, TRPM4B, transient receptor potential cation channel subfamily M, member 4), TWEAK-R, TYRP I (glycoprotein 75), VEGF, VEGF-A, EGFR-I, VEGFR-2, or Vimentin. In some embodiments, the target antigen is EphA2 or B7-H3 (CD276). In some embodiments, the target antigen is EphA2. In some embodiments, the target antigen is B7-H3 (CD276). In some embodiments, the cancer is a tumor or a hematological cancer. In some embodiments, the cancer is a breast cancer including ER positive breast cancer, multiple myeloma, plasma cell myeloma, leukemia, lymphoma, sarcoma, gastric or stomach cancer, acute myeloid leukemia, bladder cancer, brain cancer, bone marrow cancer, cervical cancer, chronic lymphocytic leukemia, colorectal cancer, pancreatic cancer, esophageal cancer, hepatocellular cancer, lymphoblastic leukemia including acute lymphoblastic leukemia, follicular lymphoma, lymphoid malignancies of T-cell or B-cell origin, metastatic castration resistant prostate cancer, bladder urothelial carcinoma, melanoma, myelogenous leukemia, myeloma, oral cancer, ovarian cancer, non-small cell lung cancer, prostate cancer, small cell lung cancer, spleen cancer, or head and neck cancer. In some embodiments, the sample is a tissue biopsy sample, a blood sample, or a bone marrow sample.

Methods of producing the described ADC compounds and compositions are also disclosed. An exemplary embodiment is a method of producing an antibody-drug conjugate by reacting an antibody or antigen-binding fragment with a cleavable conjugate linker joined or covalently attached to a panRAS inhibitor under conditions that allow conjugation.

### BRIEF DESCRIPTION OF THE DRAWINGS

FIG. 1 shows in vitro activities of panRAS ADCs, isotype ADC, and Sotorasib in multiple cancer cell lines (LU65, HPAC, H727, and SW1271).

### DETAILED DESCRIPTION OF ILLUSTRATIVE EMBODIMENTS

The disclosed compositions and methods may be understood more readily by reference to the following detailed description taken in connection with the accompanying figures, which form a part of this disclosure.

Throughout this text, the descriptions refer to compositions and methods of using the compositions. Where the disclosure describes or claims a feature or embodiment associated with a composition, such a feature or embodiment is equally applicable to the methods of using the composition. Likewise, where the disclosure describes or claims a feature or embodiment associated with a method of using a composition, such a feature or embodiment is equally applicable to the composition.

When a range of values is expressed, it includes embodiments using any particular value within the range. Further, reference to values stated in ranges includes each and every value within that range. All ranges are inclusive of their endpoints and combinable. When values are expressed as approximations, by use of the antecedent “about,” it will be understood that the particular value forms another embodiment. Reference to a particular numerical value includes at least that particular value, unless the context clearly dictates otherwise. The use of “or” will mean “and/or” unless the specific context of its use dictates otherwise. All references cited herein are incorporated by reference for any purpose. Where a reference and the specification conflict, the specification will control.

Unless the context of a description indicates otherwise, e.g., in the absence of symbols indicating specific point(s) of connectivity, when a structure or fragment of a structure is drawn, it may be used on its own or attached to other components of an ADC, and it may do so with any orientation, e.g., with the antibody or antigen-binding fragment thereof attached at any suitable attachment point to a chemical moiety such as a linker-drug. Where indicated, however, components of an ADC are attached in the orientation shown in a given formula. For example, if Formula (1) is described as Ab-(L-D)p and the group “-(L-D)” is described as

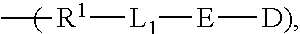

then the elaborated structure of Formula (1) is

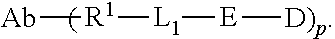

### It is not

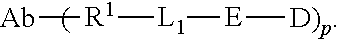

It is to be appreciated that certain features of the disclosed compositions and methods, which are, for clarity, described herein in the context of separate embodiments, may also be provided in combination in a single embodiment. Conversely, various features of the disclosed compositions and methods that are, for brevity, described in the context of a single embodiment, may also be provided separately or in any sub-combination.

As used throughout this application, antibody drug conjugates can be identified using a naming convention in the general format of “target antigen/antibody-linker-payload”. For example only, if an antibody drug conjugate is referred to as “Target X-L0-P0”, such a conjugate would comprise an antibody that binds Target X, a conjugate linker designated as L0, and a payload designated as P0. Alternatively, if an antibody drug conjugate is referred to as “anti-Target X-L0-P0”, such a conjugate would comprise an antibody that binds Target X, a conjugate linker designated as L0, and a payload designated as P0. In another alternative, if an antibody drug conjugate is referred to as “AbX-L0-P0”, such a conjugate would comprise the antibody designated as AbX, a conjugate linker designated as L0, and a payload designated as P0. A control antibody drug conjugate comprising a non-specific, isotype control antibody may be referenced as “isotype control IgG1-L0-P0” or “IgG1-L0-P0”.

Any formula given herein is also intended to represent unlabeled forms as well as isotopically labeled forms of the compounds. Isotopically labeled compounds have structures depicted by the formulae given herein except that one or more atoms are replaced by an atom having a selected atomic mass or mass number. Isotopes that can be incorporated into compounds of the invention include, for example, isotopes of hydrogen, carbon, nitrogen, oxygen, fluorine, and chlorine, such as ³H, ¹¹C ¹³C, ¹⁴C, ¹⁵N, ¹⁸F, and ³⁶Cl. Accordingly, it should be understood that the present disclosure includes compounds that incorporate one or more of any of the aforementioned isotopes, including for example, radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds can generally be prepared by conventional techniques known to those skilled in the art, e.g., using an appropriate isotopically-labeled reagents in place of the non-labeled reagent previously employed.

### Definitions

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, the singular forms “a,” “an,” and “the” include plural forms unless the context clearly dictates otherwise. The terms “comprising”, “having”, “being of” as in “being of a chemical formula”, “including”, and “containing” are to be construed as open terms (i.e., meaning “including but not limited to”) unless otherwise noted. Additionally, whenever “comprising” or another open-ended term is used in an embodiment, it is to be understood that the same embodiment can be more narrowly claimed using the intermediate term “consisting essentially of” or the closed term “consisting of”.

The term “about” or “approximately,” when used in the context of numerical values and ranges, refers to values or ranges that approximate or are close to the recited values or ranges such that the embodiment may perform as intended, as is apparent to the skilled person from the teachings contained herein. In some embodiments, about means plus or minus 20%, 15%, 10%, 5%, 1%, 0.5%, or 0.1% of a numerical amount. In one embodiment, the term “about” refers to a range of values which are 10% more or less than the specified value. In another embodiment, the term “about” refers to a range of values which are 5% more or less than the specified value. In another embodiment, the term “about” refers to a range of values which are 1% more or less than the specified value.

The terms “antibody-drug conjugate,” “antibody conjugate,” “conjugate,” “immunoconjugate,” and “ADC” are used interchangeably, and refer to one or more therapeutic compounds (e.g., a panRAS inhibitor) that is linked to one or more antibodies or antigen-binding fragments. In some embodiments, the ADC is defined by the generic formula: Ab-(L-D)ₚ (Formula 1), wherein Ab=an antibody or antigen-binding fragment (e.g., an anti-EphA2 antibody or anti-B7-H3 antibody, or an antigen-binding fragment thereof), L=a conjugate linker moiety, D=a drug moiety (e.g., a panRAS inhibitor drug moiety), and p=the number of drug moieties per antibody or antigen-binding fragment. In ADCs comprising a panRAS inhibitor drug moiety, “p” refers to the number of panRAS inhibitor compounds linked to the antibody or antigen-binding fragment.

The term “antibody” is used in the broadest sense to refer to an immunoglobulin molecule that recognizes and specifically binds to a target, such as a protein, polypeptide, carbohydrate, polynucleotide, lipid, or combinations of the foregoing through at least one antigen recognition site within the variable region of the immunoglobulin molecule. An antibody can be polyclonal or monoclonal, multiple or single chain, or an intact immunoglobulin, and may be derived from natural sources or from recombinant sources. An “intact” antibody is a glycoprotein that typically comprises at least two heavy (H) chains and two light (L) chains inter-connected by disulfide bonds. Each heavy chain is comprised of a heavy chain variable region (abbreviated herein as VH) and a heavy chain constant region. The heavy chain constant region comprises three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. An antibody can be a monoclonal antibody, human antibody, humanized antibody, camelised antibody, or chimeric antibody. The antibodies can be of any isotype (e.g., IgG, IgE, IgM, IgD, IgA and IgY), class (e.g., IgG1, IgG2, IgG3, IgG4, IgA1 and IgA2), or subclass. An antibody can be an intact antibody or an antigen-binding fragment thereof.

In some embodiments, the antibody or antibody fragment disclosed herein include modified or engineered amino acid residues, e.g., one or more cysteine residues, as sites for conjugation to a drug moiety (Junutula J R, et al., Nat Biotechnol 2008, 26:925-932). In one embodiment, the disclosure provides a modified antibody or antibody fragment comprising a substitution of one or more amino acids with cysteine at the positions described herein. Sites for cysteine substitution are in the constant regions of the antibody or antibody fragment and are thus applicable to a variety of antibody or antibody fragment, and the sites are selected to provide stable and homogeneous conjugates. A modified antibody or fragment can have one, two or more cysteine substitutions, and these substitutions can be used in combination with other modification and conjugation methods as described herein. Methods for inserting cysteine at specific locations of an antibody are known in the art, see, e.g., Lyons et al., (1990) Protein Eng., 3:703-708, WO 2011/005481, WO2014/124316, WO 2015/138615. In certain embodiments, a modified antibody comprises a substitution of one or more amino acids with cysteine on its constant region selected from positions 117, 119, 121, 124, 139, 152, 153, 155, 157, 164, 169, 171, 174, 189, 191, 195, 197, 205, 207, 246, 258, 269, 274, 286, 288, 290, 292, 293, 320, 322, 326, 333, 334, 335, 337, 344, 355, 360, 375, 382, 390, 392, 398, 400 and 422 of a heavy chain of the antibody, and wherein the positions are numbered according to the EU system. In some embodiments a modified antibody or antibody fragment comprises a substitution of one or more amino acids with cysteine on its constant region selected from positions 107, 108, 109, 114, 129, 142, 143, 145, 152, 154, 156, 159, 161, 165, 168, 169, 170, 182, 183, 197, 199, and 203 of a light chain of the antibody or antibody fragment, wherein the positions are numbered according to the EU system, and wherein the light chain is a human kappa light chain. In certain embodiments a modified antibody or antibody fragment thereof comprises a combination of substitution of two or more amino acids with cysteine on its constant regions wherein the combinations comprise substitutions at positions 375 of an antibody heavy chain, position 152 of an antibody heavy chain, position 360 of an antibody heavy chain, or position 107 of an antibody light chain and wherein the positions are numbered according to the EU system. In certain embodiments a modified antibody or antibody fragment thereof comprises a substitution of one amino acid with cysteine on its constant regions wherein the substitution is position 375 of an antibody heavy chain, position 152 of an antibody heavy chain, position 360 of an antibody heavy chain, position 107 of an antibody light chain, position 165 of an antibody light chain or position 159 of an antibody light chain and wherein the positions are numbered according to the EU system, and wherein the light chain is a kappa chain. In particular embodiments a modified antibody or antibody fragment thereof comprises a combination of substitution of two amino acids with cysteine on its constant regions wherein the combinations comprise substitutions at positions 375 of an antibody heavy chain and position 152 of an antibody heavy chain, wherein the positions are numbered according to the EU system. In particular embodiments a modified antibody or antibody fragment thereof comprises a substitution of one amino acid with cysteine at position 360 of an antibody heavy chain, wherein the positions are numbered according to the EU system. In other particular embodiments a modified antibody or antibody fragment thereof comprises a substitution of one amino acid with cysteine at position 107 of an antibody light chain and wherein the positions are numbered according to the EU system, and wherein the light chain is a kappa chain.

The term “antibody fragment” or “antigen-binding fragment” or “functional antibody fragment,” as used herein, refers to at least one portion of an antibody that retains the ability to specifically interact with (e.g., by binding, steric hinderance, stabilizing/destabilizing, spatial distribution) an epitope of an antigen (e.g., EphA2 or B7-H3 (CD276)). Antigen-binding fragments may also retain the ability to internalize into an antigen-expressing cell. In some embodiments, antigen-binding fragments also retain immune effector activity. The terms antibody, antibody fragment, antigen-binding fragment, and the like, are intended to embrace the use of binding domains from antibodies in the context of larger macromolecules such as ADCs. It has been shown that fragments of a full-length antibody can perform the antigen binding function of a full-length antibody. Examples of antibody fragments include, but are not limited to, Fab, Fab′, F(ab′)₂, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen-binding fragment can also be incorporated into single domain antibodies, maxibodies, minibodies, nanobodies, intrabodies, diabodies, triabodies, tetrabodies, bispecific or multi-specific antibody constructs, ADCs, v-NAR and bis-scFv (see, e.g., Holliger and Hudson (2005) Nat Biotechnol. 23(9):1126-36). Antigen-binding fragments can also be grafted into scaffolds based on polypeptides such as a fibronectin type Ill (Fn3) (see U.S. Pat. No. 6,703,199, which describes fibronectin polypeptide minibodies). The term “scFv” refers to a fusion protein comprising at least one antigen-binding fragment comprising a variable region of a light chain and at least one antigen-binding fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, an scFv may have the VL and VH variable regions in either order, e.g., with respect to the N-terminal and C-terminal ends of the polypeptide, the scFv may comprise VL-linker-VH or may comprise VH-linker-VL. Antigen-binding fragments are obtained using conventional techniques known to those of skill in the art, and the binding fragments are screened for utility (e.g., binding affinity, internalization) in the same manner as are intact antibodies. Antigen-binding fragments, for example, may be prepared by cleavage of the intact protein, e.g., by protease or chemical cleavage.

The term “complementarity determining region” or “CDR,” as used herein, refers to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991) “Sequences of Proteins of Immunological Interest,” 5th Ed. Public Health Service, National Institutes of Health, Bethesda, MD (“Kabat” numbering scheme); AI-Lazikani et al. (1997) J Mol Biol. 273(4):927-48 (“Chothia” numbering scheme); ImMunoGenTics (IMGT) numbering (Lefranc (2001) Nucleic Acids Res. 29(1):207-9; Lefranc et al. (2003) Dev Comp Immunol. 27(1):55-77) (“IMGT” numbering scheme); or a combination thereof. In a combined Kabat and Chothia numbering scheme for a given CDR region (for example, HC CDR1, HC CDR2, HC CDR3, LC CDR1, LC CDR2, or LC CDR3), in some embodiments, the CDRs correspond to the amino acid residues that are defined as part of the Kabat CDR, together with the amino acid residues that are defined as part of the Chothia CDR. As used herein, the CDRs defined according to the “Chothia” number scheme are also sometimes referred to as “hypervariable loops.”

In some embodiments, under Kabat, the CDR amino acid residues in the heavy chain variable domain (VH) are numbered 31-35 (HCDR1) (e.g., insertion(s) after position 35), 50-65 (HCDR2), and 95-102 (HCDR3); and the CDR amino acid residues in the light chain variable domain (VL) are numbered 24-34 (LCDR1) (e.g., insertion(s) after position 27), 50-56 (LCDR2), and 89-97 (LCDR3). In some embodiments, under Chothia, the CDR amino acids in the VH are numbered 26-32 (HCDR1) (e.g., insertion(s) after position 31), 52-56 (HCDR2), and 95-102 (HCDR3); and the amino acid residues in VL are numbered 26-32 (LCDR1) (e.g., insertion(s) after position 30), 50-52 (LCDR2), and 91-96 (LCDR3). By combining the CDR definitions of both Kabat and Chothia, in some embodiments, the CDRs comprise or consist of, e.g., amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. In some embodiments, under IMGT, the CDR amino acid residues in the VH are numbered approximately 26-35 (CDR1), 51-57 (CDR2) and 93-102 (CDR3), and the CDR amino acid residues in the VL are numbered approximately 27-32 (CDR1), 50-52 (CDR2), and 89-97 (CDR3). In some embodiments, under IMGT, the CDR regions of an antibody may be determined using the program IMGT/DomainGap Align.

The term “monoclonal antibody,” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. In contrast, conventional (polyclonal) antibody preparations typically include a multitude of antibodies directed against (or specific for) different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al. (1975) Nature 256:495, or may be made by recombinant DNA methods (see, e.g., U.S. Pat. No. 4,816,567). Monoclonal antibodies may also be isolated from phage antibody libraries using the techniques described in Clackson et al. (1991) Nature 352:624-8, and Marks et al. (1991) J Mol Biol. 222:581-97, for example. The term also includes preparations of antibody molecules of single molecular composition. A monoclonal antibody composition displays a single binding specificity and affinity for a particular epitope.

The monoclonal antibodies described herein can be non-human, human, or humanized. The term specifically includes “chimeric” antibodies, in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they specifically bind the target antigen and/or exhibit the desired biological activity.

The term “human antibody,” as used herein, refers an antibody produced by a human or an antibody having an amino acid sequence of an antibody produced by a human. The term includes antibodies having variable regions in which both the framework and CDR regions are derived from sequences of human origin. Furthermore, if the antibody contains a constant region, the constant region is also derived from such human sequences, e.g., human germline sequences, or mutated versions of human germline sequences or antibody containing consensus framework sequences derived from human framework sequences analysis, for example, as described in Knappik et al. ((2000) J Mol Biol. 296(1):57-86). The structures and locations of immunoglobulin variable domains, e.g., CDRs, may be defined using well known numbering schemes, e.g., the Kabat numbering scheme, the Chothia numbering scheme, or a combination of Kabat and Chothia, and/or ImMunoGenTics (IMGT) numbering. The human antibodies of the invention may include amino acid residues not encoded by human sequences (e.g., mutations introduced by random or site-specific mutagenesis in vitro or by somatic mutation in vivo, or a conservative substitution to promote stability or manufacturing). However, the term “human antibody,” as used herein, is not intended to include antibodies in which CDR sequences derived from the germline of another mammalian species, such as a mouse, have been grafted onto human framework sequences.

The term “recombinant human antibody,” as used herein, refers to a human antibody that is prepared, expressed, created, or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In some embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “chimeric antibody,” as used herein, refers to antibodies wherein the amino acid sequence of the immunoglobulin molecule is derived from two or more species. In some instances, the variable regions of both heavy and light chains correspond to the variable regions of antibodies derived from one species with the desired specificity, affinity, and activity while the constant regions are homologous to antibodies derived from another species (e.g., human) to minimize an immune response in the latter species.

As used herein, the term “humanized antibody” refers to forms of antibodies that contain sequences from non-human (e.g., murine) antibodies as well as human antibodies. Such antibodies are a type of chimeric antibody which contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the framework (FR) regions are those of a human immunoglobulin sequence. The humanized antibody optionally also will comprise at least a portion of an immunoglobulin constant region (Fc), typically that of a human immunoglobulin. The humanized antibody can be further modified by the substitution of residues, either in the Fv framework region and/or within the replaced non-human residues to refine and optimize antibody specificity, affinity, and/or activity.

The term “Fc region,” as used herein, refers to a polypeptide comprising the CH₃, CH₂ and at least a portion of the hinge region of a constant domain of an antibody. Optionally, an Fc region may include a CH₄ domain, present in some antibody classes. An Fc region may comprise the entire hinge region of a constant domain of an antibody. In some embodiments, an antibody or antigen-binding fragment comprises an Fc region and a CH1 region of an antibody. In some embodiments, an antibody or antigen-binding fragment comprises an Fc region CH3 region of an antibody. In some embodiments, an antibody or antigen-binding fragment comprises an Fc region, a CH1 region, and a kappa/lambda region from the constant domain of an antibody. In some embodiments, an antibody or antigen-binding fragment comprises a constant region, e.g., a heavy chain constant region and/or a light chain constant region. In some embodiments, such a constant region is modified compared to a wild-type constant region. That is, the polypeptide may comprise alterations or modifications to one or more of the three heavy chain constant domains (CH1, CH2, or CH3) and/or to the light chain constant region domain (CL). Example modifications include additions, deletions, or substitutions of one or more amino acids in one or more domains. Such changes may be included to optimize effector function, half-life, etc.

“Internalizing” as used herein in reference to an antibody or antigen-binding fragment refers to an antibody or antigen-binding fragment that is capable of being taken through the cell's lipid bilayer membrane to an internal compartment (i.e., “internalized”) upon binding to the cell, preferably into a degradative compartment in the cell. For example, an internalizing anti-EphA2 antibody is one that is capable of being taken into the cell after binding to EphA2 on the cell membrane. In some embodiments, the antibody or antigen-binding fragment used in the ADCs disclosed herein targets a cell surface antigen (e.g., EphA2 or B7-H3 (CD276)) and is an internalizing antibody or internalizing antigen-binding fragment (i.e., the ADC transfers through the cellular membrane after antigen binding). In some embodiments, the internalizing antibody or antigen-binding fragment binds a receptor on the cell surface. An internalizing antibody or internalizing antigen-binding fragment that targets a receptor on the cell membrane may induce receptor-mediated endocytosis. In some embodiments, the internalizing antibody or internalizing antigen-binding fragment is taken into the cell via receptor-mediated endocytosis.

“Non-internalizing” as used herein in reference to an antibody or antigen-binding fragment refers to an antibody or antigen-binding fragment that remains at the cell surface upon binding to the cell. In some embodiments, the antibody or antigen-binding fragment used in the ADCs disclosed herein targets a cell surface antigen and is a non-internalizing antibody or non-internalizing antigen-binding fragment (i.e., the ADC remains at the cell surface and does not transfer through the cellular membrane after antigen binding). In some embodiments, the non-internalizing antibody or antigen-binding fragment binds a non-internalizing receptor or other cell surface antigen. Exemplary non-internalizing cell surface antigens include but are not limited to CA125 and CEA, and antibodies that bind to non-internalizing antigen targets are also known in the art (see, e.g., Bast et al. (1981) J Clin Invest. 68(5):1331-7; Scholler and Urban (2007) Biomark Med. 1(4):513-23; and Boudousq et al. (2013) PLoS One 8(7): e69613).

The terms “EPH receptor A2,” “ephrin type-A receptor 2,” and “EphA2” are used interchangeably herein and refer to any native form of human EphA2. The terms encompass full-length human EphA2 (e.g., NCBI Reference Sequence: NP_004422.2; SEQ ID NO: 1), as well as any form of human EphA2 that may result from cellular processing. The terms also encompass functional variants or fragments of human EphA2, including but not limited to splice variants, allelic variants, and isoforms that retain one or more biologic functions of human EphA2 (i.e., variants and fragments are encompassed unless the context indicates that the term is used to refer to the wild-type protein only). EphA2 can be isolated from human, or may be produced recombinantly or by synthetic methods.

The term “anti-EphA2 antibody” or “antibody that binds to EphA2,” as used herein, refers to any form of antibody or antigen-binding fragment thereof that binds, e.g., specifically binds, to EphA2. The term encompasses monoclonal antibodies (including full-length monoclonal antibodies), polyclonal antibodies, and biologically functional antigen-binding fragments so long as they bind, e.g., specifically bind, to EphA2. WO2007/030642 provides and is incorporated herein by reference for exemplary EphA2-binding sequences, including exemplary anti-EphA2 antibody sequences. In some embodiments, the anti-EphA2 antibody used in the ADCs disclosed herein is an internalizing antibody or internalizing antigen-binding fragment. 1C1 (WO2007/030642) is an example of an exemplary anti-EphA2 antibody.

The terms “B7 homology 3 protein,” “B7-H3” and “CD276,” are used interchangeably herein and refer to any native form of human B7-H3 or CD276. The terms encompass full-length human B7-H3 (CD276) (e.g., NCBI Reference Sequence: NP_001019907.1), as well as any form of human B7-H3 (CD276) that may result from cellular processing. The terms also encompass functional variants or fragments of human B7-H3, including but not limited to splice variants, allelic variants, and isoforms that retain one or more biologic functions of human B7-H3 (CD276) (i.e., variants and fragments are encompassed unless the context indicates that the term is used to refer to the wild-type protein only). B7-H3 (CD276) can be isolated from human, or may be produced recombinantly or by synthetic methods.

The term “anti-B7-H3 antibody” or “antibody that binds to B7-H3 (CD276),” as used herein, refers to any form of antibody or antigen-binding fragment thereof that binds, e.g., specifically binds, to B7-H3 (CD276). The term encompasses monoclonal antibodies (including full-length monoclonal antibodies), polyclonal antibodies, and biologically functional antigen-binding fragments so long as they bind, e.g., specifically bind, to B7-H3 (CD276). WO2017214322 and WO2012147713 provide, and are incorporated herein by reference, exemplary B7-H3-binding sequences, including exemplary anti-B7-H3 (CD276) antibody sequences. ABBV-155 and DS-5573a are examples of exemplary anti-B7-H3 (CD276) antibodies.

The term “binding specificity,” as used herein, refers to the ability of an individual antibody or antigen binding fragment to preferentially react with one antigenic determinant over a different antigenic determinant. The degree of specificity indicates the extent to which an antibody or fragment preferentially binds to one antigenic determinant over a different antigenic determinant. Also, as used herein, the term “specific,” “specifically binds,” and “binds specifically” refers to a binding reaction between an antibody or antigen-binding fragment (e.g., an anti-EphA2 antibody or an anti-B7-H3 antibody) and a target antigen (e.g., EphA2 or B7-H3 (CD276)) in a heterogeneous population of proteins and other biologics. Antibodies can be tested for specificity of binding by comparing binding to an appropriate antigen to binding to an irrelevant antigen or antigen mixture under a given set of conditions. If the antibody binds to the appropriate antigen with at least 2, 5, 7, 10 or more times more affinity than to the irrelevant antigen or antigen mixture, then it is considered to be specific. A “specific antibody” or a “target-specific antibody” is one that only binds the target antigen (e.g., EphA2 or B7-H3 (CD276)), but does not bind (or exhibits minimal binding) to other antigens. In some embodiments, an antibody or antigen-binding fragment that specifically binds a target antigen (e.g., EphA2 or B7-H3 (CD276)) has a K_{D} of less than 1×10⁻⁶ M, less than 1×10⁻⁷ M, less than 1×10⁻⁸ M, less than 1×10⁻⁹ M, less than 1×10⁻¹⁰ M, less than 1×10⁻¹¹ M, less than 1×10⁻¹² M, or less than 1×10⁻¹³ M. In some embodiments, the K_{D} is 1 pM to 500 pM. In some embodiments, the K_{D} is between 500 pM to 1 μM, 1 μM to 100 nM, or 100 mM to 10 nM.

The term “affinity,” as used herein, refers to the strength of interaction between antibody and antigen at single antigenic sites. Without being bound by theory, within each antigen binding site, the variable region of the antibody “arm” interacts through weak non-covalent forces with the antigen at numerous sites; the more interactions, typically the stronger the affinity. The binding affinity of an antibody is the sum of the attractive and repulsive forces operating between the antigenic determinant and the binding site of the antibody.

The term “kₒₙ” or “kₐ” refers to the on-rate constant for association of an antibody to the antigen to form the antibody/antigen complex. The rate can be determined using standard assays, such as a surface plasmon resonance, biolayer inferometry, or ELISA assay.

The term “k_{off}” or “k_{d}” refers to the off-rate constant for dissociation of an antibody from the antibody/antigen complex. The rate can be determined using standard assays, such as a surface plasmon resonance, biolayer inferometry, or ELISA assay.

The term “K_{D}” refers to the equilibrium dissociation constant of a particular antibody-antigen interaction. K_{D} is calculated by kₐ/k_{d}. The rate can be determined using standard assays, such as a surface plasmon resonance, biolayer inferometry, or ELISA assay.

The term “epitope” refers to the portion of an antigen capable of being recognized and specifically bound by an antibody (or antigen-binding fragment). Epitope determinants generally consist of chemically active surface groupings of molecules such as amino acids or carbohydrate or sugar side chains and can have specific three-dimensional structural characteristics, as well as specific charge characteristics. When the antigen is a polypeptide, epitopes can be formed from contiguous amino acids or noncontiguous amino acids juxtaposed by tertiary folding of the polypeptide. An epitope may be “linear” or “conformational.” Conformational and linear epitopes are distinguished in that the binding to the former but not the latter is lost in the presence of denaturing solvents. The epitope bound by an antibody (or antigen-binding fragment) may be identified using any epitope mapping technique known in the art, including X-ray crystallography for epitope identification by direct visualization of the antigen-antibody complex, as well as monitoring the binding of the antibody to fragments or mutated variations of the antigen, or monitoring solvent accessibility of different parts of the antibody and the antigen. Exemplary strategies used to map antibody epitopes include, but are not limited to, array-based oligo-peptide scanning, limited proteolysis, site-directed mutagenesis, high-throughput mutagenesis mapping, hydrogen-deuterium exchange, and mass spectrometry (see, e.g., Gershoni et al. (2007) BioDrugs 21:145-56; and Hager-Braun and Tomer (2005) Expert Rev Proteomics 2:745-56).

Competitive binding and epitope binning can also be used to determine antibodies sharing identical or overlapping epitopes. Competitive binding can be evaluated using a cross-blocking assay, such as the assay described in “Antibodies, A Laboratory Manual,” Cold Spring Harbor Laboratory, Harlow and Lane (1^{st} edition 1988, 2^{nd} edition 2014). In some embodiments, competitive binding is identified when a test antibody or binding protein reduces binding of a reference antibody or binding protein to a target antigen such as EphA2 or B7-H3 (CD276) (e.g., a binding protein comprising CDRs and/or variable domains selected from those identified in Tables 3-5), by at least about 50% in the cross-blocking assay (e.g., 50%, 60%, 70%, 80%, 90%, 95%, 99%, 99.5%, or more, or any percentage in between), and/or vice versa. In some embodiments, competitive binding can be due to shared or similar (e.g., partially overlapping) epitopes, or due to steric hindrance where antibodies or binding proteins bind at nearby epitopes (see, e.g., Tzartos, Methods in Molecular Biology (Morris, ed. (1998) vol. 66, pp. 55-66)). In some embodiments, competitive binding can be used to sort groups of binding proteins that share similar epitopes. For example, binding proteins that compete for binding can be “binned” as a group of binding proteins that have overlapping or nearby epitopes, while those that do not compete are placed in a separate group of binding proteins that do not have overlapping or nearby epitopes.

As used herein, the terms “peptide,” “polypeptide,” and “protein” are used interchangeably to refer to a polymer of amino acid residues. The terms encompass amino acid polymers comprising two or more amino acids joined to each other by peptide bonds, amino acid polymers in which one or more amino acid residues is an artificial chemical mimetic of a corresponding naturally-occurring amino acid, as well as naturally-occurring amino acid polymers and non-naturally-occurring amino acid polymers. The terms include, for example, biologically active fragments, substantially homologous polypeptides, oligopeptides, homodimers, heterodimers, variants of polypeptides, modified polypeptides, derivatives, analogs, fusion proteins, among others. The terms also include natural peptides, recombinant peptides, synthetic peptides, or a combination thereof. Unless otherwise indicated, a particular polypeptide sequence also implicitly encompasses conservatively modified variants thereof.

A “recombinant” protein refers to a protein (e.g., an antibody) made using recombinant techniques, e.g., through the expression of a recombinant nucleic acid.

An “isolated” protein refers to a protein unaccompanied by at least some of the material with which it is normally associated in its natural state. For example, a naturally-occurring polynucleotide or polypeptide present in a living organism is not isolated, but the same polynucleotide or polypeptide separated from some or all of the coexisting materials in the living organism, is isolated. The definition includes the production of an antibody in a wide variety of organisms and/or host cells that are known in the art.

An “isolated antibody,” as used herein, is an antibody that has been identified and separated from one or more (e.g., the majority) of the components (by weight) of its source environment, e.g., from the components of a hybridoma cell culture or a different cell culture that was used for its production. In some embodiments, the separation is performed such that it sufficiently removes components that may otherwise interfere with the suitability of the antibody for the desired applications (e.g., for therapeutic use). Methods for preparing isolated antibodies are known in the art and include, without limitation, protein A chromatography, anion exchange chromatography, cation exchange chromatography, virus retentive filtration, and ultrafiltration.

As used herein, the term “variant” refers to a nucleic acid sequence or an amino acid sequence that differs from a reference nucleic acid sequence or amino acid sequence respectively, but retains one or more biological properties of the reference sequence. A variant may contain one or more amino acid substitutions, deletions, and/or insertions (or corresponding substitution, deletion, and/or insertion of codons) with respect to a reference sequence. Changes in a nucleic acid variant may not alter the amino acid sequence of a peptide encoded by the reference nucleic acid sequence, or may result in amino acid substitutions, additions, deletions, fusions, and/or truncations. In some embodiments, a nucleic acid variant disclosed herein encodes an identical amino acid sequence to that encoded by the unmodified nucleic acid or encodes a modified amino acid sequence that retains one or more functional properties of the unmodified amino acid sequence. Changes in the sequence of peptide variants are typically limited or conservative, so that the sequences of the unmodified peptide and the variant are closely similar overall and, in many regions, identical. In some embodiments, a peptide variant retains one or more functional properties of the unmodified peptide sequence. A variant and unmodified peptide can differ in amino acid sequence by one or more substitutions, additions, deletions in any combination.

A variant of a nucleic acid or peptide can be a naturally-occurring variant or a variant that is not known to occur naturally. Variants of nucleic acids and peptides may be made by mutagenesis techniques, by direct synthesis, or by other techniques known in the art. A variant does not necessarily require physical manipulation of the reference sequence. As long as a sequence contains a different nucleic acid or amino acid as compared to a reference sequence, it is considered a “variant” regardless of how it was synthesized. In some embodiments, a variant has high sequence identity (i.e., 60% nucleic acid or amino acid sequence identity or higher) as compared to a reference sequence. In some embodiments, a peptide variant encompasses polypeptides having amino acid substitutions, deletions, and/or insertions as long as the polypeptide has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% amino acid sequence identity with a reference sequence, or with a corresponding segment (e.g., a functional fragment) of a reference sequence, e.g., those variants that also retain one or more functions of the reference sequence. In some embodiments, a nucleic acid variant encompasses polynucleotides having amino acid substitutions, deletions, and/or insertions as long as the polynucleotide has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% nucleic acid sequence identity with a reference sequence, or with a corresponding segment (e.g., a functional fragment) of a reference sequence.

The term “conservatively modified variant” applies to both amino acid and nucleic acid sequences. For nucleic acid sequences, conservatively modified variants refer to those nucleic acids which encode identical or essentially identical amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence. For polypeptide sequences, conservatively modified variants include individual substitutions, deletions, or additions to a polypeptide sequence which result in the substitution of an amino acid with a chemically similar amino acid. Conservative substitutions providing functionally similar amino acids are well known in the art.

The term “conservative sequence modifications,” as used herein, refers to amino acid modifications that do not significantly affect or alter the binding characteristics of, e.g., an antibody or antigen-binding fragment containing the amino acid sequence. Such conservative modifications include amino acid substitutions, additions, and deletions. Modifications can be introduced into an antibody or antigen-binding fragment by standard techniques known in the art, such as, e.g., site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, in some embodiments, one or more amino acid residues within an antibody can be replaced with other amino acid residues from the same side chain family and the altered antibody can be tested using the functional assays described herein.

The term “homologous” or “identity,” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions. For example, if half (e.g., five positions in a polymer ten subunits in length) of the positions in two sequences are matched or homologous, the two sequences are 50% homologous; if 90% of the positions (e.g., 9 of 10), are matched or homologous, the two sequences are 90% homologous.

Percentage of “sequence identity” can be determined by comparing two optimally aligned sequences over a comparison window, where the fragment of the amino acid sequence in the comparison window may comprise additions or deletions (e.g., gaps or overhangs) as compared to the reference sequence (which does not comprise additions or deletions) for optimal alignment of the two sequences. The percentage can be calculated by determining the number of positions at which the identical amino acid residue occurs in both sequences to yield the number of matched positions, dividing the number of matched positions by the total number of positions in the window of comparison, and multiplying the result by 100 to yield the percentage of sequence identity. The output is the percent identity of the subject sequence with respect to the query sequence. The percent identity between the two sequences is a function of the number of identical positions shared by the sequences, taking into account the number of gaps, and the length of each gap, which need to be introduced for optimal alignment of the two sequences. Generally, the amino acid identity or homology between proteins disclosed herein and variants thereof, including variants of target antigens (such as EphA2 or B7-H3 (CD276)) and variants of antibody variable domains (including individual variant CDRs), is at least 80% to the sequences depicted herein, e.g., identities or homologies of at least 85%, 90%, 91%, 92%, 93%, 94%, 95%, 96%, 97%, 98%, 99%, almost 100%, or 100%.

The comparison of sequences and determination of percent identity between two sequences can be accomplished using a mathematical algorithm. In some embodiments, the percent identity between two amino acid sequences is determined using the Needleman and Wunsch ((1970) J Mol Biol. 48:444-53) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In some embodiments, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. An exemplary set of parameters is a Blossum 62 scoring matrix with a gap penalty of 12, a gap extend penalty of 4, and a frameshift gap penalty of 5. The percent identity between two amino acid or nucleotide sequences can also be determined using the algorithm of Meyers and Miller ((1989) CABIOS 4:11-17) which has been incorporated into the ALIGN program (version 2.0), using a PAM120 weight residue table, a gap length penalty of 12 and a gap penalty of 4.

The term “agent” is used herein to refer to a chemical compound, a mixture of chemical compounds, a biological macromolecule, an extract made from biological materials, or a combination of two or more thereof. The term “therapeutic agent” or “drug” refers to an agent that is capable of modulating a biological process and/or has biological activity. The panRAS inhibitors and the ADCs comprising them, as described herein, are exemplary therapeutic agents.

The term “chemotherapeutic agent” or “anti-cancer agent” is used herein to refer to all agents that are effective in treating cancer (regardless of mechanism of action). Inhibition of metastasis or angiogenesis is frequently a property of a chemotherapeutic agent. Chemotherapeutic agents include antibodies, biological molecules, and small molecules, and encompass the panRAS inhibitors and ADCs comprising them, as described herein. A chemotherapeutic agent may be a cytotoxic or cytostatic agent. The term “cytostatic agent” refers to an agent that inhibits or suppresses cell growth and/or multiplication of cells. The term “cytotoxic agent” refers to a substance that causes cell death primarily by interfering with a cell's expression activity and/or functioning.

The term “Rat Sarcoma Virus (Ras)” or “panRAS,” as used herein, refers to any native form of the human Ras protein family (e.g., K-Ras (including splice variants KRAS4A and KRAS4B), H-Ras and N-Ras). The term encompasses full-length human K-Ras (Kristen Rat Sarcoma Virus) (e.g., UniProt Reference Sequence: P01116; SEQ ID NO:64), H-Ras (Harvey Rat Sarcoma Virus) (e.g., UniProt Reference Sequence: P01112; SEQ ID NO:65), N-Ras (Neuroblastoma Rat Sarcoma Virus) (e.g., UniProt Reference Sequence: P01111; SEQ ID NO:66), as well as any form of human Ras that may result from cellular processing. The term also encompasses functional variants or fragments of human Ras proteins, including but not limited to splice variants, allelic variants, and isoforms that retain one or more biologic functions of human Ras proteins (i.e., variants and fragments are encompassed unless the context indicates that the term is used to refer to the wild-type protein only). Ras proteins can be isolated from human, or may be produced recombinantly or by synthetic methods. Exemplary Ras protein amino acid sequences are listed in Table C below.

TABLE C
Exemplary RAS amino acid sequences

| Ras Protein | SEQ ID NO | Amino acid sequence |
|---|---|---|
| K-Ras | 64 | MTEYKLVVVGAGGVGKSALTIQLIQNHFVDEYDPTIEDSYRKQVV |
| | | IDGETCLLDILDTAGQEEYSAMRDQYMRTGEGFLCVFAINNTKSF |
| | | EDIHHYREQIKRVKDSEDVPMVLVGNKCDLPSRTVDTKQAQDLAR |
| | | SYGIPFIETSAKTRQRVEDAFYTLVREIRQYRLKKISKEEKTPGC |
| | | VKIKKCIIM |
| H-Ras | 65 | MTEYKLVVVGAGGVGKSALTIQLIQNHFVDEYDPTIEDSYRKQVV |
| | | IDGETCLLDILDTAGQEEYSAMRDQYMRTGEGFLCVFAINNTKSF |
| | | EDIHQYREQIKRVKDSDDVPMVLVGNKCDLAARTVESRQAQDLAR |
| | | SYGIPYIETSAKTRQGVEDAFYTLVREIRQHKLRKLNPPDESGPG |
| | | CMSCKCVLS |
| N-Ras | 66 | MTEYKLVVVGAGGVGKSALTIQLIQNHFVDEYDPTIEDSYRKQVV |
| | | IDGETCLLDILDTAGQEEYSAMRDQYMRTGEGFLCVFAINNSKSF |
| | | ADINLYREQIKRVKDSDDVPMVLVGNKCDLPTRTVDTKQAHELAK |
| | | SYGIPFIETSAKTRQGVEDAFYTLVREIRQYRMKKLNSSDDGTQG |
| | | CMGLPCVVM |

The term “inhibit” or “inhibition” or “inhibiting,” as used herein, means to reduce a biological activity or process by a measurable amount, and can include but does not require complete prevention or inhibition. In some embodiments, “inhibition” means to reduce the expression and/or activity of panRAS and/or one or more upstream modulators or downstream targets thereof.

The term “panRAS inhibitor,” as used herein, refers to an agent capable of reducing the expression and/or activity of panRAS (e.g., K-Ras (including splice variants KRAS4A and KRAS4B), H-Ras and N-Ras) and/or one or more upstream modulators or downstream targets thereof. Exemplary panRAS modulators (including exemplary inhibitors of panRAS) are described in WO2021/091956 or WO2022/060836, each of which are incorporated herein by reference as exemplary panRAS modulators, including exemplary panRAS inhibitors, that can be included as drug moieties in the disclosed ADCs.

As used herein, a “panRAS inhibitor drug moiety”, “panRAS inhibitor”, and the like refer to the component of an ADC or composition that provides the structure of a panRAS inhibitor compound or a compound modified for attachment to an ADC that retains essentially the same, similar, or enhanced biological function or activity as compared to the original compound. In some embodiments, panRAS inhibitor drug moiety is component (D) in an ADC of Formula (1).

The term “cancer,” as used herein, refers to the presence of cells possessing characteristics typical of cancer-causing cells, such as uncontrolled proliferation, immortality, metastatic potential, rapid growth and proliferation rate, and/or certain morphological features. Often, cancer cells can be in the form of a tumor or mass, but such cells may exist alone within a subject, or may circulate in the blood stream as independent cells, such as leukemic or lymphoma cells. The term “cancer” includes all types of cancers and cancer metastases, including hematological cancers, solid tumors, sarcomas, carcinomas and other solid and non-solid tumor cancers. Hematological cancers may include B-cell malignancies, cancers of the blood (leukemias), cancers of plasma cells (myelomas, e.g., multiple myeloma), or cancers of the lymph nodes (lymphomas). Exemplary B-cell malignancies include chronic lymphocytic leukemia (CLL), follicular lymphoma, mantle cell lymphoma, and diffuse large B-cell lymphoma. Leukemias may include acute lymphoblastic leukemia (ALL), acute myeloid leukemia (AML), chronic lymphocytic leukemia (CLL), chronic myelogenous leukemia (CML), chronic myelomonocytic leukemia (CMML), acute monocytic leukemia (AMoL), etc. The terms “acute lymphoblastic leukemia” and “acute lymphocytic leukemia” can be used interchangeably to describe ALL. Lymphomas may include Hodgkin's lymphoma, non-Hodgkin's lymphoma, etc. Other hematologic cancers may include myelodysplasia syndrome (MDS). Solid tumors may include carcinomas such as adenocarcinoma, e.g., a breast cancer including ER positive breast cancer, multiple myeloma, plasma cell myeloma, leukemia, lymphoma, sarcoma, gastric or stomach cancer, acute myeloid leukemia, bladder cancer, brain cancer, bone marrow cancer, cervical cancer, chronic lymphocytic leukemia, colorectal cancer, pancreatic cancer, esophageal cancer, hepatocellular cancer, lymphoblastic leukemia including acute lymphoblastic leukemia, follicular lymphoma, lymphoid malignancies of T-cell or B-cell origin, metastatic castration resistant prostate cancer, bladder urothelial carcinoma, melanoma, myelogenous leukemia, myeloma, oral cancer, ovarian cancer, non-small cell lung cancer, prostate cancer, small cell lung cancer, spleen cancer, or head and neck cancer.

As used herein, the term “tumor” refers to any mass of tissue that results from excessive cell growth or proliferation, either benign or malignant, including precancerous lesions. In some embodiments, the tumor is a breast cancer including ER positive breast cancer, multiple myeloma, plasma cell myeloma, leukemia, lymphoma, sarcoma, gastric or stomach cancer, acute myeloid leukemia, bladder cancer, brain cancer, bone marrow cancer, cervical cancer, chronic lymphocytic leukemia, colorectal cancer, pancreatic cancer, esophageal cancer, hepatocellular cancer, lymphoblastic leukemia including acute lymphoblastic leukemia, follicular lymphoma, lymphoid malignancies of T-cell or B-cell origin, metastatic castration resistant prostate cancer, bladder urothelial carcinoma, melanoma, myelogenous leukemia, myeloma, oral cancer, ovarian cancer, non-small cell lung cancer, prostate cancer, small cell lung cancer, spleen cancer, or head and neck cancer.

The terms “tumor cell” and “cancer cell” may be used interchangeably herein and refer to individual cells or the total population of cells derived from a tumor or cancer, including both non-tumorigenic cells and cancer stem cells. The terms “tumor cell” and “cancer cell” will be modified by the term “non-tumorigenic” when referring solely to those cells lacking the capacity to renew and differentiate to distinguish those cells from cancer stem cells.

The term “target-negative,” “target antigen-negative,” or “antigen-negative,” as used herein, refers to the absence of target antigen expression by a cell or tissue. The term “target-positive,” “target antigen-positive,” or “antigen-positive” refers to the presence of target antigen expression. For example, a cell or a cell line that does not express a target antigen may be described as target-negative, whereas a cell or cell line that expresses a target antigen may be described as target-positive.

The terms “subject” and “patient” are used interchangeably herein to refer to any human or non-human animal in need of treatment. Non-human animals include all vertebrates (e.g., mammals and non-mammals) such as any mammal. Non-limiting examples of mammals include humans, chimpanzees, apes, monkeys, cattle, horses, sheep, goats, swine, rabbits, dogs, cats, rats, mice, and guinea pigs. Non-limiting examples of non-mammals include birds and fish. In some embodiments, the subject is a human.

The term “a subject in need of treatment,” as used herein, refers to a subject that would benefit biologically, medically, or in quality of life from a treatment (e.g., a treatment with any one or more of the exemplary ADC compounds described herein).

As used herein, the term “treat,” “treating,” or “treatment” refers to any improvement of any consequence of disease, disorder, or condition, such as prolonged survival, less morbidity, and/or a lessening of side effects which result from an alternative therapeutic modality. In some embodiments, treatment comprises delaying or ameliorating a disease, disorder, or condition (i.e., slowing or arresting or reducing the development of a disease or at least one of the clinical symptoms thereof). In some embodiments, treatment comprises delaying, alleviating, or ameliorating at least one physical parameter of a disease, disorder, or condition, including those which may not be discernible by the patient. In some embodiments, treatment comprises modulating a disease, disorder, or condition, either physically (e.g., stabilization of a discernible symptom), physiologically (e.g., stabilization of a physical parameter), or both. In some embodiments, treatment comprises administration of a described ADC compound or composition to a subject, e.g., a patient, to obtain a treatment benefit enumerated herein. The treatment can be to cure, heal, alleviate, delay, prevent, relieve, alter, remedy, ameliorate, palliate, improve, or affect a disease, disorder, or condition (e.g., a cancer), the symptoms of a disease, disorder, or condition (e.g., a cancer), or a predisposition toward a disease, disorder, or condition (e.g., a cancer). In some embodiments, in addition to treating a subject having a disease, disorder, or condition, a composition disclosed herein can also be provided prophylactically to prevent or reduce the likelihood of developing that disease, disorder, or condition.

As used herein, the term “prevent”, “preventing,” or “prevention” of a disease, disorder, or condition refers to the prophylactic treatment of the disease, disorder, or condition; or delaying the onset or progression of the disease, disorder, or condition.

As used herein, a “pharmaceutical composition” refers to a preparation of a composition, e.g., an ADC compound or composition, in addition to at least one other (and optionally more than one other) component suitable for administration to a subject, such as a pharmaceutically acceptable carrier, stabilizer, diluent, dispersing agent, suspending agent, thickening agent, and/or excipient. The pharmaceutical compositions provided herein are in such form as to permit administration and subsequently provide the intended biological activity of the active ingredient(s) and/or to achieve a therapeutic effect. The pharmaceutical compositions provided herein preferably contain no additional components which are unacceptably toxic to a subject to which the formulation would be administered.

As used herein, the terms “pharmaceutically acceptable carrier” and “physiologically acceptable carrier,” which may be used interchangeably, refer to a carrier or a diluent that does not cause significant irritation to a subject and does not abrogate the biological activity and properties of the administered ADC compound or composition and/or any additional therapeutic agent in the composition. Pharmaceutically acceptable carriers may enhance or stabilize the composition or can be used to facilitate preparation of the composition. Pharmaceutically acceptable carriers can include solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated. The carrier may be selected to minimize adverse side effects in the subject, and/or to minimize degradation of the active ingredient(s). An adjuvant may also be included in any of these formulations.

As used herein, the term “excipient” refers to an inert substance added to a pharmaceutical composition to further facilitate administration of an active ingredient. Formulations for parenteral administration can, for example, contain excipients such as sterile water or saline, polyalkylene glycols such as polyethylene glycol, vegetable oils, or hydrogenated napthalenes. Other exemplary excipients include, but are not limited to, calcium bicarbonate, calcium phosphate, various sugars and types of starch, cellulose derivatives, gelatin, ethylene-vinyl acetate co-polymer particles, and surfactants, including, for example, polysorbate 20.

The term “pharmaceutically acceptable salt,” as used herein, refers to a salt which does not abrogate the biological activity and properties of the compounds of the invention, and does not cause significant irritation to a subject to which it is administered. Examples of such salts include, but are not limited to: (a) acid addition salts formed with inorganic acids, for example, hydrochloric acid, hydrobromic acid, sulfuric acid, phosphoric acid, nitric acid and the like; and salts formed with organic acids, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (b) salts formed from elemental anions such as chlorine, bromine, and iodine. See, e.g., Haynes et al., “Commentary: Occurrence of Pharmaceutically Acceptable Anions and Cations in the Cambridge Structural Database,” J. Pharmaceutical Sciences, vol. 94, no. 10 (2005), and Berge et al., “Pharmaceutical Salts,” J. Pharmaceutical Sciences, vol. 66, no. 1 (1977), which are incorporated by reference herein.

In some embodiments, depending on their electronic charge, the antibody-drug conjugates (ADCs), conjugate linkers, payloads and linker-payloads described herein can contain a monovalent anionic counterion M₁⁻. Any suitable anionic counterion can be used. In certain embodiments, the monovalent anionic counterion is a pharmaceutically acceptable monovalent anionic counterion. In certain embodiments, the monovalent anionic counterion M₁⁻ can be selected from bromide, chloride, iodide, acetate, trifluoroacetate, benzoate, mesylate, tosylate, triflate, formate, or the like. In some embodiments, the monovalent anionic counterion M₁⁻ is trifluoroacetate or formate.

As used herein, the term “therapeutically effective amount” or “therapeutically effective dose,” refers to an amount of a compound described herein, e.g., an ADC compound or composition described herein, to effect the desired therapeutic result (i.e., reduction or inhibition of an enzyme or a protein activity, amelioration of symptoms, alleviation of symptoms or conditions, delay of disease progression, a reduction in tumor size, inhibition of tumor growth, prevention of metastasis). In some embodiments, a therapeutically effective amount does not induce or cause undesirable side effects. In some embodiments, a therapeutically effective amount induces or causes side effects but only those that are acceptable by a treating clinician in view of a patient's condition. In some embodiments, a therapeutically effective amount is effective for detectable killing, reduction, and/or inhibition of the growth or spread of cancer cells, the size or number of tumors, and/or other measure of the level, stage, progression and/or severity of a cancer. The term also applies to a dose that will induce a particular response in target cells, e.g., a reduction, slowing, or inhibition of cell growth. A therapeutically effective amount can be determined by first administering a low dose, and then incrementally increasing that dose until the desired effect is achieved. A therapeutically effective amount can also vary depending upon the intended application (in vitro or in vivo), or the subject and disease condition being treated, e.g., the weight and age of the subject, the severity of the disease condition, the manner of administration and the like, which can readily be determined by one of ordinary skill in the art. The specific amount may vary depending on, for example, the particular pharmaceutical composition, the subject and their age and existing health conditions or risk for health conditions, the dosing regimen to be followed, the severity of the disease, whether it is administered in combination with other agents, timing of administration, the tissue to which it is administered, and the physical delivery system in which it is carried. In the case of cancer, a therapeutically effective amount of an ADC may reduce the number of cancer cells, reduce tumor size, inhibit (e.g., slow or stop) tumor metastasis, inhibit (e.g., slow or stop) tumor growth, and/or relieve one or more symptoms.

As used herein, the term “prophylactically effective amount” or “prophylactically effective dose,” refers to an amount of a compound disclosed herein, e.g., an ADC compound or composition described herein, that is effective, at dosages and for periods of time necessary, to achieve the desired prophylactic result. Typically, since a prophylactic dose is used in subjects prior to or at an earlier stage of disease, the prophylactically effective amount will be less than the therapeutically effective amount. In some embodiments, a prophylactically effective amount can prevent the onset of disease symptoms, including symptoms associated with a cancer.

The term “p” or “drug loading” or “drug:antibody ratio” or “drug-to-antibody ratio” or “DAR” refers to the number of drug moieties per antibody or antigen-binding fragment, i.e., drug loading, or the number of -L-D moieties per antibody or antigen-binding fragment (Ab) in ADCs of Formula (1). In ADCs comprising a panRAS inhibitor drug moiety, “p” refers to the number of panRAS inhibitor compounds linked to the antibody or antigen-binding fragment. For example, if two panRAS inhibitor compounds are linked to an antibody or antigen-binding fragment, p=2. In compositions comprising multiple copies of ADCs of Formula (1), “average p” refers to the average number of -L-D moieties per antibody or antigen-binding fragment, also referred to as “average drug loading.”

### Antibody-Drug Conjugates

The antibody-drug conjugate (ADC) compounds of the present disclosure include those with anti-cancer activity. In particular, the ADC compounds include an antibody or antigen-binding fragment conjugated (i.e., covalently attached by a conjugate linker) to a drug moiety (e.g., a panRAS inhibitor), wherein the drug moiety when not conjugated to an antibody or antigen-binding fragment has a cytotoxic or cytostatic effect. In some embodiments, the drug moiety when not conjugated to an antibody or antigen-binding fragment is capable of reducing the expression and/or activity of panRAS and/or one or more upstream modulators or downstream targets thereof. Without being bound by theory, by targeting panRAS expression and/or activity, in some embodiments, the ADCs disclosed herein may provide potent anti-cancer agents. Also, without being bound by theory, by conjugating the drug moiety to an antibody that binds an antigen associated with expression in a tumor cell or cancer, the ADC may provide improved activity, better cytotoxic specificity, and/or reduced off-target killing as compared to the drug moiety when administered alone.

In some embodiments, therefore, the components of the ADC are selected to (i) retain one or more therapeutic properties exhibited by the antibody and drug moieties in isolation, (ii) maintain the specific binding properties of the antibody or antigen-binding fragment; (iii) optimize drug loading and drug-to-antibody ratios; (iv) allow delivery, e.g., intracellular delivery, of the drug moiety via stable attachment to the antibody or antigen-binding fragment; (v) retain ADC stability as an intact conjugate until transport or delivery to a target site; (vi) minimize aggregation of the ADC prior to or after administration; (vii) allow for the therapeutic effect, e.g., cytotoxic effect, of the drug moiety after cleavage or other release mechanism in the cellular environment; (viii) exhibit in vivo anti-cancer treatment efficacy comparable to or superior to that of the antibody and drug moieties in isolation; (ix) minimize off-target killing by the drug moiety; and/or (x) exhibit desirable pharmacokinetic and pharmacodynamics properties, formulatability, and toxicologic/immunologic profiles. Each of these properties may provide for an improved ADC for therapeutic use (Ab et al. (2015) Mol Cancer Ther. 14:1605-13).

The ADC compounds of the present disclosure may selectively deliver an effective dose of a cytotoxic or cytostatic agent to cancer cells or to tumor tissue. In some embodiments, the cytotoxic and/or cytostatic activity of the ADC is dependent on target antigen expression in a cell. In some embodiments, the disclosed ADCs are particularly effective at killing cancer cells expressing a target antigen while minimizing off-target killing. In some embodiments, the disclosed ADCs do not exhibit a cytotoxic and/or cytostatic effect on cancer cells that do not express a target antigen.

Provided herein, in certain aspects, are ADC compounds comprising an antibody or antigen-binding fragment thereof (Ab), a panRAS inhibitor drug moiety (D), and a conjugate linker moiety (L) that covalently attaches Ab to D. In some embodiments, provided herein, are ADC compounds comprising an antibody or antigen-binding fragment thereof (Ab) which targets a cancer cell, a panRAS inhibitor drug moiety (D), and a conjugate linker moiety (L) that covalently attaches Ab to D. In some embodiments, the antibody or antigen-binding fragment is able to bind to a tumor-associated antigen (e.g., EphA2 or B7-H3 (CD276)), e.g., with high specificity and high affinity. In some embodiments, the antibody or antigen-binding fragment is internalized into a target cell upon binding, e.g., into a degradative compartment in the cell. In some embodiments, the ADCs internalize upon binding to a target cell, undergo degradation, and release the panRAS inhibitor drug moiety to kill cancer cells. The panRAS inhibitor drug moiety may be released from the antibody and/or the conjugate linker moiety of the ADC by enzymatic action, hydrolysis, oxidation, or any other mechanism.

An exemplary ADC has Formula (1):

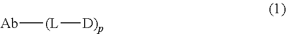

wherein Ab=an antibody or antigen-binding fragment, L=a conjugate linker moiety, D=a panRAS inhibitor drug moiety, and p=the number of panRAS inhibitor drug moieties per antibody or antigen-binding fragment.

### Antibodies

The antibody or antigen-binding fragment (Ab) of Formula (1) includes within its scope any antibody or antigen-binding fragment that specifically binds to a target antigen on a cell. In some embodiments, the target antigen is BCMA, CD33, HER2, CD38, CD48, CD79b, PCAD, CD74, CD138, SLAMF7, CD123, CLL1, FLT3, CD7, CKIT, CD56, DLL3, DLK1, B7-H3, B7-H4, EGFR, CD71, EPCAM, FOLR1, ENPP3, MET, AXL, SLC34A2 (NaPi2b), Nectin4, TROP2, LIV1, CD46, MSLN, CD142 (F3), MUC1, MUC16, SLC39A6, TFRC, TACSTD2, GPNMB, EphA2, CD56, SEZ6, CD25, CCR8, CEACAM5, CEACAM6, 4-1BB, 5AC, 5T4, Alpha-fetoprotein, angiopoietin 2, ASLG659, TCLI, BMPRIB, Brevican BCAN, BEHAB, C242 antigen, C5, CA-125, CA-125 (imitation), CA-IX (Carbonic anhydrase 9), CCR4, CD140a, CD152, CD19, CD20, CD200, CD21 (C3DR) I), CD22 (B-cell receptor CD22-B isoform), CD221, CD23 (gE receptor), CD28, CD30 (TNFRSF8), CD37, CD4, CD40, CD44 v6, CD51, CD52, CD70, CD72 (Lyb-2, B-cell differentiation antigen CD72), CD79a, CD80, CD166 (ALCAM), CDH17, CA9, CEA, CEA-related antigen, ch4D5, CLDN18.2, CRIPTO (CR, CRI, CRGF, TDGF1, CFC1B), CTLA-4, CXCR5, DLL4, DR5, E16 (LATI, SLC7A5), EGFL7, EphB2R (DRT, ERK, Hek5, EPHT3, Tyro5), Episialin, ERBB3, ETBR (Endothelin type B receptor), FCRHI (Fc receptor-like protein I), FcRH2 (IFGP4, IRTA4, SPAPI, SPAP IB, SPAP IC), Fibronectin extra domain-B, Frizzled receptor, GD2, GD3 ganglioside, GEDA, HER1, HER2/neu, HER3, HGF, HLA-DOB, HLA-DR, Human scatter factor receptor kinase, IGF-I receptor, IL-13, IL20R (ZCYTOR7), IL-6, ILGF2, ILFRIR, integrin u, IRTA2 (Immunoglobulin superfamily receptor translocation associated 2), Lewis-Y antigen, LY64 (RP105), LY6E, STEAP1, ADAM9, PTK7, MMP14, TM4SF1, ITGB6, FXYD5, MCP-I, MDP (DPEPI), MPF, MSLN, SMR, mesothelin, megakaryocyte, PD-I, PDCDI, PDGF-R u, Prostate specific membrane antigen (PSMA), PSCA (Prostate stem cell antigen precursor), PRLR (Prolactin Receptor), PSCA hlg, RANKL, RON, SDCI, Sema Sb, STEAP I, STEAP2, PCANAP I, STAMP I, STEAP2, STMP, prostate cancer associated gene I, TAG-72, TEMI, Tenascin C, TENB2, (TMEFF2, tomoregulin, TPEF, HPPI, TR), TGF-IJ, TRAIL-E2, TRAIL-RI, TRAIL-R2, T17M4 (BR22450, FLJ20041, TRPM4, TRPM4B, transient receptor potential cation channel subfamily M, member 4), TWEAK-R, TYRP I (glycoprotein 75), VEGF, VEGF-A, EGFR-I, VEGFR-2, or Vimentin. In some embodiments, the target antigen is EphA2 or B7-H3. In some embodiments, the antibody or antigen-binding fragment (Ab) of Formula (1) includes within its scope any antibody or antigen-binding fragment that specifically binds to a target antigen on a cancer cell. In some embodiments, said cell or said cancer cell expresses EphA2. In some embodiments, the target antigen EphA2 has the amino acid sequence described in Table 6.

In some embodiments, the target antigen B7-H3 (CD276) has the amino acid sequence described in Table 6.

The antibody or antigen-binding fragment (e.g., anti-EphA2 or anti-B7-H3 antibody or antigen-binding fragment) may bind to a target antigen with a dissociation constant (K_{D}) of ≤1 mM, ≤100 nM or ≤10 nM, or any amount in between, as measured by, e.g., BIAcore® analysis. In some embodiments, the K_{D} is 1 pM to 500 pM. In some embodiments, the K_{D} is between 500 pM to 1 μM, 1 μM to 100 nM, or 100 mM to 10 nM.

In some embodiments, the antibody or antigen-binding fragment (e.g., anti-EphA2 or anti-B7-H3 antibody or antigen-binding fragment) is a four-chain antibody (also referred to as an immunoglobulin or a full-length or intact antibody), comprising two heavy chains and two light chains. In some embodiments, the antibody or antigen-binding fragment (e.g., anti-EphA2 or anti-B7-H3 antibody or antigen-binding fragment) is an antigen-binding fragment of an immunoglobulin. In some embodiments, the antibody or antigen-binding fragment (e.g., anti-EphA2 or anti-B7-H3 antibody or antigen-binding fragment) is an antigen-binding fragment of an immunoglobulin that retains the ability to bind a target cancer antigen and/or provide at least one function of the immunoglobulin.

In some embodiments, the antibody or antigen-binding fragment (e.g., anti-EphA2 or anti-B7-H3 antibody or antigen-binding fragment) is an internalizing antibody or internalizing antigen-binding fragment thereof. In some embodiments, the internalizing antibody (e.g. anti-EphA2 antibody or anti-B7-H3 antibody) or internalizing antigen-binding fragment thereof (e.g., anti-EphA2 or anti-B7-H3 antigen-binding fragment) binds to a target cancer antigen expressed on the surface of a cell and enters the cell upon binding. In some embodiments, the panRAS inhibitor drug moiety of the ADC is released from the antibody or antigen-binding fragment (e.g., anti-EphA2 or anti-B7-H3 antibody or antigen-binding fragment) of the ADC after the ADC enters and is present in a cell expressing the target cancer antigen (i.e., after the ADC has been internalized), e.g., by cleavage, by degradation of the antibody or antigen-binding fragment, or by any other suitable release mechanism.

In some embodiments, the antibodies (e.g. anti-EphA2 antibody or anti-B7-H3 antibody) comprise mutations that mediate reduced or no antibody-dependent cellular cytotoxicity (ADCC) or complement-dependent cytotoxicity (CDC). In some embodiments, these mutations are known as Fc Silencing, Fc Silent, or Fc Silenced mutations. In some embodiments, amino acid residues L234 and L235 of the IgG1 constant region are substituted to A234 and A235 (also known as “LALA”). In some embodiments, amino acid residue N297 of the IgG1 constant region is substituted to A297 (also known as “N297A”). In some embodiments, amino acid residues D265 and P329 of the IgG1 constant region are substituted to A265 and A329 (also known as “DAPA”). Other antibody Fc silencing mutations may also be used. In some embodiments, the Fc silencing mutations are used in combination, for example D265A, N297A and P329A (also known as “DANAPA”).

Amino acid sequences of exemplary antibodies of the present disclosure, in addition to exemplary antigen targets, are set forth in Tables 2-6.

TABLE 2
Antibodies Exemplified

| Antibody Target | Antibody Code | mAb Reference |
|---|---|---|
| EphA2 | EphA2 CysMab | 1C1 |
| B7-H3 | B7-H3 CysMab | ABBV-155 |
| B7-H3 | B7-H3 | DS-5573a |

TABLE 3
Amino acid sequences of mAb variable regions

| | | SEQ | |
|---|---|---|---|
| | IgG | ID | |
| mAb | chain | NO | Amino acid sequence |
| EphA2 | VH | 11 | EVQLLESGGGLVQPGGSLRLSCAASGFTF |
| 1C1 | | | SHYMMAWVRQAPGKGLEWVSRIGPSGGPT |
| | | | HYADSVKGRFTISRDNSKNTLYLQMNSLR |
| | | | AEDTAVYYCAGYDSGYDYVAVAGPAEYFQ |
| | | | HWGQGTLVTVSS |
| EphA2 | VL | 12 | DIQMTQSPSSLSASVGDRVTITCRASQSI |
| 1C1 | | | STWLAWYQQKPGKAPKLLIYKASNLHTGV |
| | | | PSRFSGSGSGTEFSLTISGLQPDDFATYY |
| | | | CQQYNSYSRTFGQGTKVEIK |
| B7-H3 | VH | 13 | EVQLVQSGAEVKKPGSSVKVSCKASGYTF |
| ABBV-155 | | | SSYWMHWVRQAPGQGLEWIGLIHPESGST |
| | | | NYNEMFKNRATLTVDRSTSTAYMELSSLR |
| | | | SEDTAVYYCAGGGRLYFDYWGQGTTVTVS |
| | | | S |
| B7-H3 | VL | 14 | DIVMTQSPLSLPVTPGEPASISCRSSQSL |
| ABBV-155 | | | VHSNRDTYLRWYLQKPGQSPQLLIYKVSN |
| | | | RFSGVPDRESGSGSGTDFTLKISRVEAED |
| | | | VGVYYCSQSTHVPYTFGGGTKVEIK |
| B7-H3 | VH | 15 | QVQLVQSGAEVKKPGSSVKVSCKASGYTF |
| DS-5573a | | | TNYVMHWVRQAPGQGLEWMGYINPYNDDV |
| | | | KYNEKFKGRVTITADESTSTAYMELSSLR |
| | | | SEDTAVYYCARWGYYGSPLYYFDYWGQGT |
| | | | LVTVSS |
| B7-H3 | VL | 16 | EIVLTQSPATLSLSPGERATLSCRASSRL |
| DS-5573a | | | IYMHWYQQKPGQAPRPLIYATSNLASGIP |
| | | | ARFSGSGSGTDFTLTISSLEPEDFAVYYC |
| | | | QQWNSNPPTFGQGTKVEIK |

TABLE 4
Amino acid sequences of mAb CDRs (Combined)

| | | | |
|---|---|---|---|
| | | SEQ | |
| | Numbering | ID | |
| CDR | Scheme | NO | Amino acid sequence |

EphA2 1C1

| | | | |
|---|---|---|---|
| HCDR1 | Chothia | 17 | GFTFSHY |
| HCDR2 | Chothia | 18 | GPSGGP |
| HCDR3 | Chothia | 19 | YDSGYDYVAVAGPAEYFQH |
| HCDR1 | Kabat | 20 | HYMMA |
| HCDR2 | Kabat | 21 | RIGPSGGPTHYADSVKG |
| HCDR3 | Kabat | 19 | YDSGYDYVAVAGPAEYFQH |
| HCDR1 | IMGT | 22 | GFTFSHYM |
| HCDR2 | IMGT | 23 | IGPSGGPT |
| HCDR3 | IMGT | 24 | AGYDSGYDYVAVAGPAEYFQH |
| HCDR1 | Combined | 25 | GFTFSHYMMA |
| HCDR2 | Combined | 21 | RIGPSGGPTHYADSVKG |
| HCDR3 | Combined | 19 | YDSGYDYVAVAGPAEYFQH |
| LCDR1 | Chothia | 26 | SQSISTW |
| LCDR2 | Chothia | 27 | KAS |
| LCDR3 | Chothia | 28 | YNSYSR |
| LCDR1 | Kabat | 29 | RASQSISTWLA |
| LCDR2 | Kabat | 30 | KASNLHT |
| LCDR3 | Kabat | 31 | QQYNSYSRT |
| LCDR1 | IMGT | 32 | QSISTW |
| LCDR2 | IMGT | 27 | KAS |
| LCDR3 | IMGT | 31 | QQYNSYSRT |
| LCDR1 | Combined | 29 | RASQSISTWLA |
| LCDR2 | Combined | 30 | KASNLHT |
| LCDR3 | Combined | 31 | QQYNSYSRT |

B7-H3 (CD276) ABBV-155

| | | | |
|---|---|---|---|
| HCDR1 | Chothia | 33 | GYTFSSY |
| HCDR2 | Chothia | 34 | HPESGS |
| HCDR3 | Chothia | 35 | GGRLYFDY |
| HCDR1 | Kabat | 36 | SYWMH |
| HCDR2 | Kabat | 37 | LIHPESGSTNYNEMFKN |
| HCDR3 | Kabat | 35 | GGRLYFDY |
| HCDR1 | IMGT | 38 | GYTFSSYW |
| HCDR2 | IMGT | 39 | IHPESGST |
| HCDR3 | IMGT | 40 | AGGGRLYFDY |
| HCDR1 | Combined | 41 | GYTFSSYWMH |
| HCDR2 | Combined | 37 | LIHPESGSTNYNEMFKN |
| HCDR3 | Combined | 35 | GGRLYFDY |
| LCDR1 | Chothia | 42 | SQSLVHSNRDTY |
| LCDR2 | Chothia | 43 | KVS |
| LCDR3 | Chothia | 44 | STHVPY |
| LCDR1 | Kabat | 45 | RSSQSLVHSNRDTYLR |
| LCDR2 | Kabat | 46 | KVSNRFS |
| LCDR3 | Kabat | 47 | SQSTHVPYT |
| LCDR1 | IMGT | 48 | QSLVHSNRDTY |
| LCDR2 | IMGT | 43 | KVS |
| LCDR3 | IMGT | 47 | SQSTHVPYT |
| LCDR1 | Combined | 45 | RSSQSLVHSNRDTYLR |
| LCDR2 | Combined | 46 | KVSNRFS |
| LCDR3 | Combined | 47 | SQSTHVPYT |

B7-H3 (CD276) DS-5573a

| | | | |
|---|---|---|---|
| HCDR1 | Chothia | 49 | GYTFTNY |
| HCDR2 | Chothia | 50 | NPYNDD |
| HCDR3 | Chothia | 51 | WGYYGSPLYYFDY |
| HCDR1 | Kabat | 52 | NYVMH |
| HCDR2 | Kabat | 53 | YINPYNDDVKYNEKFKG |
| HCDR3 | Kabat | 51 | WGYYGSPLYYFDY |
| HCDR1 | IMGT | 54 | GYTFTNYV |
| HCDR2 | IMGT | 55 | INPYNDDV |
| HCDR3 | IMGT | 56 | ARWGYYGSPLYYFDY |
| HCDR1 | Combined | 57 | GYTFTNYVMH |
| HCDR2 | Combined | 53 | YINPYNDDVKYNEKFKG |
| HCDR3 | Combined | 51 | WGYYGSPLYYFDY |
| LCDR1 | Chothia | 58 | SSRLIY |
| LCDR2 | Chothia | 59 | ATS |
| LCDR3 | Chothia | 60 | WNSNPP |
| LCDR1 | Kabat | 61 | RASSRLIYMH |
| LCDR2 | Kabat | 62 | ATSNLAS |
| LCDR3 | Kabat | 63 | QQWNSNPPT |
| LCDR1 | IMGT | 58 | SRLIY |
| LCDR2 | IMGT | 59 | ATS |
| LCDR3 | IMGT | 63 | QQWNSNPPT |
| LCDR1 | Combined | 61 | RASSRLIYMH |
| LCDR2 | Combined | 62 | ATSNLAS |
| LCDR3 | Combined | 63 | QQWNSNPPT |

TABLE 5
Amino acid and nucleic acid sequences of full-length mAb Ig chains

| mAb | IgG chain | SEQ ID NO | Amino acid sequence |
|---|---|---|---|
| EphA2 1C1 | Heavy Chain | 3 | EVQLLESGGGLVQPGGSLRLSCAASGFT |
| | (Wild Type | | FSHYMMAWVRQAPGKGLEWVSRIGPSGG |
| | Fc) | | PTHYADSVKGRFTISRDNSKNTLYLQMN |
| | | | SLRAEDTAVYYCAGYDSGYDYVAVAGPA |
| | | | EYFQHWGQGTLVTVSSASTKGPSVFPLA |
| | | | PSSKSTSGGTAALGCLVKDYFPCPVTVS |
| | | | WNSGALTSGVHTFPAVLQSSGLYSLSSV |
| | | | VTVPSSSLGTQTYICNVNHKPSNTKVDK |
| | | | RVEPKSCDKTHTCPPCPAPELLGGPSVF |
| | | | LFPPKPKDTLMISRTPEVTCVVVDVSHE |
| | | | DPEVKFNWYVDGVEVHNAKTKPREEQYN |
| | | | STYRVVSVLTVLHQDWLNGKEYKCKVSN |
| | | | KALPAPIEKTISKAKGQPREPQVYTLPP |
| | | | SREEMTKNQVSLTCLVKGFYPCDIAVEW |
| | | | ESNGQPENNYKTTPPVLDSDGSFFLYSK |
| | | | LTVDKSRWQQGNVFSCSVMHEALHNHYT |
| | | | QKSLSLSPGK |
| | DNA Heavy | 4 | GAAGTTCAGCTGCTTGAATCTGGCGGCG |
| | Chain | | GACTGGTTCAACCTGGCGGATCTCTGAG |
| | | | ACTGAGCTGTGCCGCCAGCGGCTTCACC |
| | | | TTCAGCCACTATATGATGGCCTGGGTCC |
| | | | GACAGGCCCCTGGCAAAGGACTTGAATG |
| | | | GGTGTCCAGAATCGGCCCCTCTGGCGGC |
| | | | CCTACACACTACGCTGATTCTGTGAAGG |
| | | | GCAGATTCACCATCAGCCGGGACAACAG |
| | | | CAAGAACACCCTGTACCTGCAGATGAAC |
| | | | AGCCTGAGAGCCGAGGACACCGCCGTGT |
| | | | ATTACTGTGCCGGCTACGACAGCGGCTA |
| | | | CGATTATGTGGCTGTGGCCGGACCTGCC |
| | | | GAGTACTTTCAGCATTGGGGACAGGGCA |
| | | | CCCTGGTCACCGTTAGTTCTGCTAGCAC |
| | | | CAAGGGCCCAAGTGTGTTTCCCCTGGCC |
| | | | CCCAGCAGCAAGTCTACTTCCGGCGGAA |
| | | | CTGCTGCCCTGGGTTGCCTGGTGAAGGA |
| | | | CTACTTCCCCTGTCCCGTGACAGTGTCC |
| | | | TGGAACTCTGGGGCTCTGACTTCCGGCG |
| | | | TGCACACCTTCCCCGCCGTGCTGCAGAG |
| | | | CAGCGGCCTGTACAGCCTGAGCAGCGTG |
| | | | GTGACAGTGCCCTCCAGCTCTCTGGGAA |
| | | | CCCAGACCTATATCTGCAACGTGAACCA |
| | | | CAAGCCCAGCAACACCAAGGTGGACAAG |
| | | | AGAGTGGAGCCCAAGAGCTGCGACAAGA |
| | | | CCCACACCTGCCCCCCCTGCCCAGCTCC |
| | | | AGAACTGCTGGGAGGGCCTTCCGTGTTC |
| | | | CTGTTCCCCCCCAAGCCCAAGGACACCC |
| | | | TGATGATCAGCAGGACCCCCGAGGTGAC |
| | | | CTGCGTGGTGGTGGACGTGTCCCACGAG |
| | | | GACCCAGAGGTGAAGTTCAACTGGTACG |
| | | | TGGACGGCGTGGAGGTGCACAACGCCAA |
| | | | GACCAAGCCCAGAGAGGAGCAGTACAAC |
| | | | AGCACCTACAGGGTGGTGTCCGTGCTGA |
| | | | CCGTGCTGCACCAGGACTGGCTGAACGG |
| | | | CAAAGAATACAAGTGCAAAGTCTCCAAC |
| | | | AAGGCCCTGCCAGCCCCAATCGAAAAGA |
| | | | CAATCAGCAAGGCCAAGGGCCAGCCACG |
| | | | GGAGCCCCAGGTGTACACCCTGCCCCCC |
| | | | AGCCGGGAGGAGATGACCAAGAACCAGG |
| | | | TGTCCCTGACCTGTCTGGTGAAGGGCTT |
| | | | CTACCCCTGTGATATCGCCGTGGAGTGG |
| | | | GAGAGCAACGGCCAGCCCGAGAACAACT |
| | | | ACAAGACCACCCCCCCAGTGCTGGACAG |
| | | | CGACGGCAGCTTCTTCCTGTACAGCAAG |
| | | | CTGACCGTGGACAAGTCCAGGTGGCAGC |
| | | | AGGGCAACGTGTTCAGCTGCAGCGTGAT |
| | | | GCACGAGGCCCTGCACAACCACTACACC |
| | | | CAGAAGTCCCTGAGCCTGAGCCCCGGCA |
| | | | AG |
| EphA2 1C1 | Light Chain | 5 | DIQMTQSPSSLSASVGDRVTITCRASQS |
| | | | ISTWLAWYQQKPGKAPKLLIYKASNLHT |
| | | | GVPSRFSGSGSGTEFSLTISGLQPDDFA |
| | | | TYYCQQYNSYSRTFGQGTKVEIKRTVAA |
| | | | PSVFIFPPSDEQLKSGTASVVCLLNNFY |
| | | | PREAKVQWKVDNALQSGNSQESVTEQDS |
| | | | KDSTYSLSSTLTLSKADYEKHKVYACEV |
| | | | THQGLSSPVTKSFNRGEC |
| | DNA Light | 6 | GACATTCAGATGACACAGAGCCCTAGCA |
| | Chain | | GCCTGAGCGCCTCTGTGGGAGACAGAGT |
| | | | GACCATCACCTGTAGAGCCAGCCAGAGC |
| | | | ATCAGCACATGGCTGGCCTGGTATCAGC |
| | | | AGAAGCCTGGCAAGGCCCCTAAGCTGCT |
| | | | GATCTACAAGGCCAGCAATCTGCACACC |
| | | | GGCGTGCCCAGCAGATTTTCTGGCTCTG |
| | | | GAAGCGGCACCGAGTTCAGCCTGACAAT |
| | | | CTCTGGCCTGCAGCCTGACGACTTCGCC |
| | | | ACCTACTACTGCCAGCAGTACAACAGCT |
| | | | ACAGCCGGACCTTTGGCCAGGGAACAAA |
| | | | GGTGGAAATCAAGCGTACGGTGGCCGCT |
| | | | CCCAGCGTGTTCATCTTCCCCCCCAGCG |
| | | | ACGAGCAGCTGAAGAGTGGCACCGCCAG |
| | | | CGTGGTGTGCCTGCTGAACAACTTCTAC |
| | | | CCCCGGGAGGCCAAGGTGCAGTGGAAGG |
| | | | TGGACAACGCCCTGCAGAGCGGCAACAG |
| | | | CCAGGAGAGCGTCACCGAGCAGGACAGC |
| | | | AAGGACTCCACCTACAGCCTGAGCAGCA |
| | | | CCCTGACCCTGAGCAAGGCCGACTACGA |
| | | | GAAGCATAAGGTGTACGCCTGCGAGGTG |
| | | | ACCCACCAGGGCCTGTCCAGCCCCGTGA |
| | | | CCAAGAGCTTCAACAGGGGCGAGTGC |
| B7-H3 | Heavy chain | 7 | EVQLVQSGAEVKKPGSSVKVSCKASGYT |
| ABBV-155 | | | FSSYWMHWVRQAPGQGLEWIGLIHPESG |
| | | | STNYNEMFKNRATLTVDRSTSTAYMELS |
| | | | SLRSEDTAVYYCAGGGRLYFDYWGQGTT |
| | | | VTVSSASTKGPSVFPLAPSSKSTSGGTA |
| | | | ALGCLVKDYFPCPVTVSWNSGALTSGVH |
| | | | TFPAVLQSSGLYSLSSVVTVPSSSLGTQ |
| | | | TYICNVNHKPSNTKVDKRVEPKSCDKTH |
| | | | TCPPCPAPELLGGPSVFLFPPKPKDTLM |
| | | | ISRTPEVTCVVVDVSHEDPEVKFNWYVD |
| | | | GVEVHNAKTKPREEQYNSTYRVVSVLTV |
| | | | LHQDWLNGKEYKCKVSNKALPAPIEKTI |
| | | | SKAKGQPREPQVYTLPPSREEMTKNQVS |
| | | | LTCLVKGFYPCDIAVEWESNGQPENNYK |
| | | | TTPPVLDSDGSFFLYSKLTVDKSRWQQG |
| | | | NVFSCSVMHEALHNHYTQKSLSLSPGK |
| B7-H3 | Light chain | 8 | DIVMTQSPLSLPVTPGEPASISCRSSQS |
| ABBV-155 | | | LVHSNRDTYLRWYLQKPGQSPQLLIYKV |
| | | | SNRFSGVPDRFSGSGSGTDFTLKISRVE |
| | | | AEDVGVYYCSQSTHVPYTFGGGTKVEIK |
| | | | RTVAAPSVFIFPPSDEQLKSGTASVVCL |
| | | | LNNFYPREAKVQWKVDNALQSGNSQESV |
| | | | TEQDSKDSTYSLSSTLTLSKADYEKHKV |
| | | | YACEVTHQGLSSPVTKSFNRGEC |
| B7-H3 | Heavy Chain | 9 | QVQLVQSGAEVKKPGSSVKVSCKASGYT |
| DS-5573a | | | FTNYVMHWVRQAPGOGLEWMGYINPYND |
| | | | DVKYNEKFKGRVTITADESTSTAYMELS |
| | | | SLRSEDTAVYYCARWGYYGSPLYYFDYW |
| | | | GQGTLVTVSSASTKGPSVFPLAPSSKST |
| | | | SGGTAALGCLVKDYFPCPVTVSWNSGAL |
| | | | TSGVHTFPAVLQSSGLYSLSSVVTVPSS |
| | | | SLGTQTYICNVNHKPSNTKVDKRVEPKS |
| | | | CDKTHTCPPCPAPELLGGPSVFLFPPKP |
| | | | KDTLMISRTPEVTCVVVDVSHEDPEVKF |
| | | | NWYVDGVEVHNAKTKPREEQYNSTYRVV |
| | | | SVLTVLHQDWLNGKEYKCKVSNKALPAP |
| | | | IEKTISKAKGQPREPQVYTLPPSREEMT |
| | | | KNQVSLTCLVKGFYPCDIAVEWESNGQP |
| | | | ENNYKTTPPVLDSDGSFFLYSKLTVDKS |
| | | | RWQQGNVFSCSVMHEALHNHYTQKSLSL |
| | | | SPGK |
| B7-H3 | Light Chain | 10 | EIVLTQSPATLSLSPGERATLSCRASSR |
| DS-5573a | | | LIYMHWYQQKPGQAPRPLIYATSNLASG |
| | | | IPARFSGSGSGTDFTLTISSLEPEDFAV |
| | | | YYCQQWNSNPPTFGQGTKVEIKRTVAAP |
| | | | SVFIFPPSDEQLKSGTASVVCLLNNFYP |
| | | | REAKVQWKVDNALQSGNSQESVTEQDSK |
| | | | DSTYSLSSTLTLSKADYEKHKVYACEVT |
| | | | HQGLSSPVTKSFNRGEC |

TABLE 6
Exemplary target antigen amino acid sequences

| Antigen | SEQ ID NO | Amino acid sequence |
|---|---|---|
| EphA2 | 1 | MELQAARACFALLWGCALAAAAAAQGKEVVLLDFAAAGGELG |
| | | WLTHPYGKGWDLMQNIMNDMPIYMYSVCNVMSGDQDNWLRTN |
| | | WVYRGEAERIFIELKFTVRDCNSFPGGASSCKETFNLYYAES |
| | | DLDYGTNFQKRLFTKIDTIAPDEITVSSDFEARHVKLNVEER |
| | | SVGPLTRKGFYLAFQDIGACVALLSVRVYYKKCPELLQGLAH |
| | | FPETIAGSDAPSLATVAGTCVDHAVVPPGGEEPRMHCAVDGE |
| | | WLVPIGQCLCQAGYEKVEDACQACSPGFFKFEASESPCLECP |
| | | EHTLPSPEGATSCECEEGFFRAPQDPASMPCTRPPSAPHYLT |
| | | AVGMGAKVELRWTPPQDSGGREDIVYSVTCEQCWPESGECGP |
| | | CEASVRYSEPPHGLTRTSVTVSDLEPHMNYTFTVEARNGVSG |
| | | LVTSRSFRTASVSINQTEPPKVRLEGRSTTSLSVSWSIPPPQ |
| | | QSRVWKYEVTYRKKGDSNSYNVRRTEGFSVTLDDLAPDTTYL |
| | | VQVQALTQEGQGAGSKVHEFQTLSPEGSGNLAVIGGVAVGVV |
| | | LLLVLAGVGFFIHRRRKNQRARQSPEDVYFSKSEQLKPLKTY |
| | | VDPHTYEDPNQAVLKFTTEIHPSCVTRQKVIGAGEFGEVYKG |
| | | MLKTSSGKKEVPVAIKTLKAGYTEKQRVDFLGEAGIMGQFSH |
| | | HNIIRLEGVISKYKPMMIITEYMENGALDKFLREKDGEFSVL |
| | | QLVGMLRGIAAGMKYLANMNYVHRDLAARNILVNSNLVCKVS |
| | | DFGLSRVLEDDPEATYTTSGGKIPIRWTAPEAISYRKFTSAS |
| | | DVWSFGIVMWEVMTYGERPYWELSNHEVMKAINDGFRLPTPM |
| | | DCPSAIYQLMMQCWQQERARRPKFADIVSILDKLIRAPDSLK |
| | | TLADFDPRVSIRLPSTSGSEGVPFRTVSEWLESIKMQQYTEH |
| | | FMAAGYTAIEKVVQMINDDIKRIGVRLPGHQKRIAYSLLGLK |
| | | DQVNTVGIPI |
| B7-H3 | 2 | MLRRRGSPGMGVHVGAALGALWFCLTGALEVQVPEDPVVALV |
| | | GTDATLCCSFSPEPGESLAQLNLIWQLTDTKQLVHSFAEGQD |
| | | QGSAYANRTALFPDLLAQGNASLRLQRVRVADEGSFTCFVSI |
| | | RDFGSAAVSLQVAAPYSKPSMTLEPNKDLRPGDTVTITCSSY |
| | | QGYPEAEVFWQDGQGVPLTGNVTTSQMANEQGLFDVHSILRV |
| | | VLGANGTYSCLVRNPVLQQDAHSSVTITPQRSPTGAVEVQVP |
| | | EDPVVALVGTDATLRCSFSPEPGFSLAQLNLIWOLTDTKQLV |
| | | HSFTEGRDQGSAYANRTALFPDLLAQGNASLRLQRVRVADEG |
| | | SFTCFVSIRDFGSAAVSLQVAAPYSKPSMTLEPNKDLRPGDT |
| | | VTITCSSYRGYPEAEVFWQDGQGVPLTGNVTTSQMANEQGLF |
| | | DVHSVLRVVLGANGTYSCLVRNPVLQQDAHGSVTITGQPMTF |
| | | PPEALWVTVGLSVCLIALLVALAFVCWRKIKQSCEEENAGAE |
| | | DODGEGEGSKTALQPLKHSDSKEDDGQEIA |

In some embodiments, the antibody or antigen-binding fragment of an ADC disclosed herein may comprise any set of heavy and light chain variable domains listed in the tables above or a set of six CDRs from any set of heavy and light chain variable domains listed in the tables above. In some embodiments, the antibody or antigen-binding fragment of an ADC disclosed herein may comprise amino acid sequences that are conservatively modified and/or homologous to the sequences listed in the tables above, so long as the ADC retains the ability to bind to its target cancer antigen (e.g., with a K_{D} of less than 1×10⁻⁸ M) and retains one or more functional properties of the ADCs disclosed herein (e.g., ability to internalize, bind to an antigen target, e.g., an antigen expressed on a tumor or other cancer cell, etc.).

In some embodiments, the antibody or antigen-binding fragment of an ADC disclosed herein further comprises human heavy and light chain constant domains or fragments thereof. For instance, the antibody or antigen-binding fragment of the described ADCs may comprise a human IgG heavy chain constant domain (such as an IgG1) and a human kappa or lambda light chain constant domain. In some embodiments, the antibody or antigen-binding fragment of the described ADCs comprises a human immunoglobulin G subtype 1 (IgG1) heavy chain constant domain with a human Ig kappa light chain constant domain.

In some embodiments, the target cancer antigen for an ADC is EphA2.

In some embodiments, the anti-EphA2 antibody or antigen-binding fragment of an ADC disclosed herein further comprises human heavy and light chain constant domains or fragments thereof. For instance, the anti-EphA2 antibody or antigen-binding fragment of the described ADCs may comprise a human IgG heavy chain constant domain (such as an IgG1) and a human kappa or lambda light chain constant domain. In some embodiments, the anti-EphA2 antibody or antigen-binding fragment of the described ADCs comprises a human immunoglobulin G subtype 1 (IgG1) heavy chain constant domain with a human Ig kappa light chain constant domain.

In some embodiments, the anti-EphA2 antibody or antigen-binding fragment thereof comprises three heavy chain CDRs and three light chain CDRs as follows: heavy chain CDR1 (HCDR1) consisting of SEQ ID NO:17, heavy chain CDR2 (HCDR2) consisting of SEQ ID NO:18, heavy chain CDR3 (HCDR3) consisting of SEQ ID NO:19; light chain CDR1 (LCDR1) consisting of SEQ ID NO:26, light chain CDR2 (LCDR2) consisting of SEQ ID NO:27, and light chain CDR3 (LCDR3) consisting of SEQ ID NO:28.

In some embodiments, the anti-EphA2 antibody or antigen-binding fragment thereof comprises three heavy chain CDRs and three light chain CDRs as follows: heavy chain CDR1 (HCDR1) consisting of SEQ ID NO:20, heavy chain CDR2 (HCDR2) consisting of SEQ ID NO:21, heavy chain CDR3 (HCDR3) consisting of SEQ ID NO:19; light chain CDR1 (LCDR1) consisting of SEQ ID NO:29, light chain CDR2 (LCDR2) consisting of SEQ ID NO:30, and light chain CDR3 (LCDR3) consisting of SEQ ID NO:31.

In some embodiments, the anti-EphA2 antibody or antigen-binding fragment thereof comprises three heavy chain CDRs and three light chain CDRs as follows: heavy chain CDR1 (HCDR1) consisting of SEQ ID NO:22, heavy chain CDR2 (HCDR2) consisting of SEQ ID NO:23, heavy chain CDR3 (HCDR3) consisting of SEQ ID NO:24; light chain CDR1 (LCDR1) consisting of SEQ ID NO:32, light chain CDR2 (LCDR2) consisting of SEQ ID NO:27, and light chain CDR3 (LCDR3) consisting of SEQ ID NO:31.

In some embodiments, the anti-EphA2 antibody or antigen-binding fragment thereof comprises three heavy chain CDRs and three light chain CDRs as follows: heavy chain CDR1 (HCDR1) consisting of SEQ ID NO:25, heavy chain CDR2 (HCDR2) consisting of SEQ ID NO:21, heavy chain CDR3 (HCDR3) consisting of SEQ ID NO:19; light chain CDR1 (LCDR1) consisting of SEQ ID NO:29, light chain CDR2 (LCDR2) consisting of SEQ ID NO:30, and light chain CDR3 (LCDR3) consisting of SEQ ID NO:31.

In some embodiments, the anti-EphA2 antibody or antigen-binding fragment thereof comprises the three heavy chain CDRs and three light chain CDRs, wherein the CDRs include no more than one, two, three, four, five, or six amino acid additions, deletions or substitutions of HCDR1 (SEQ ID NO:17), HCDR2 (SEQ ID NO:18), HCDR3 (SEQ ID NO:19); LCDR1 (SEQ ID NO:26), LCDR2 (SEQ ID NO:27), and LCDR3 (SEQ ID NO:28).

In some embodiments, the anti-EphA2 antibody or antigen-binding fragment thereof comprises the three heavy chain CDRs and three light chain CDRs, wherein the CDRs include no more than one, two, three, four, five, or six amino acid additions, deletions or substitutions of HCDR1 (SEQ ID NO:20), HCDR2 (SEQ ID NO:21), HCDR3 (SEQ ID NO:19); LCDR1 (SEQ ID NO:29), LCDR2 (SEQ ID NO:30), and LCDR3 (SEQ ID NO:31).

In some embodiments, the anti-EphA2 antibody or antigen-binding fragment thereof comprises the three heavy chain CDRs and three light chain CDRs, wherein the CDRs include no more than one, two, three, four, five, or six amino acid additions, deletions or substitutions of HCDR1 (SEQ ID NO:22), HCDR2 (SEQ ID NO:23), HCDR3 (SEQ ID NO:24); LCDR1 (SEQ ID NO:32), LCDR2 (SEQ ID NO:27), and LCDR3 (SEQ ID NO:31).

In some embodiments, the anti-EphA2 antibody or antigen-binding fragment thereof comprises the three heavy chain CDRs and three light chain CDRs, wherein the CDRs include no more than one, two, three, four, five, or six amino acid additions, deletions or substitutions of HCDR1 (SEQ ID NO:25), HCDR2 (SEQ ID NO:21), HCDR3 (SEQ ID NO:19); LCDR1 (SEQ ID NO:29), LCDR2 (SEQ ID NO:30), and LCDR3 (SEQ ID NO:31).

In some embodiments, the anti-EphA2 antibody or antigen-binding fragment thereof comprises the heavy chain variable region amino acid sequence of SEQ ID NO: 11 and the light chain variable region amino acid sequence of SEQ ID NO:12. In some embodiments, the anti-EphA2 antibody or antigen-binding fragment thereof comprises the heavy chain variable region amino acid sequence of SEQ ID NO: 11 and the light chain variable region amino acid sequence of SEQ ID NO:12, or sequences that are at least 95% identical to the disclosed sequences. In some embodiments, the anti-EphA2 antibody or antigen-binding fragment thereof has a heavy chain variable region amino acid sequence that is at least 96%, at least 97%, at least 98%, or at least 99% identical to SEQ ID NO: 11 and/or a light chain variable region amino acid sequence that is at least 96%, at least 97%, at least 98%, or at least 99% identical to SEQ ID NO:12.

In some embodiments, the anti-EphA2 antibody comprises the heavy chain amino acid sequence of SEQ ID NO:3 or a sequence that is at least 95% identical to SEQ ID NO:3, and the light chain amino acid sequence of SEQ ID NO:5 or a sequence that is at least 95% identical to SEQ ID NO:5. In some embodiments, the anti-EphA2 antibody comprises the heavy chain amino acid sequence of SEQ ID NO:3 and the light chain amino acid sequence of SEQ ID NO:5, or sequences that are at least 95% identical to the disclosed sequences. In some embodiments, the anti-EphA2 antibody has a heavy chain amino acid sequence that is at least 96%, at least 97%, at least 98%, or at least 99% identical to SEQ ID NO:3 and a light chain amino acid sequence that is at least 96%, at least 97%, at least 98%, or at least 99% identical to SEQ ID NO:5.

In some embodiments, the target cancer antigen for an ADC is B7-H3 (CD276).

In some embodiments, the anti-B7-H3 (CD276) antibody or antigen-binding fragment of an ADC disclosed herein further comprises human heavy and light chain constant domains or fragments thereof. For instance, the anti-B7-H3 (CD276) antibody or antigen-binding fragment of the described ADCs may comprise a human IgG heavy chain constant domain (such as an IgG1) and a human kappa or lambda light chain constant domain. In some embodiments, the anti-B7-H3 (CD276) antibody or antigen-binding fragment of the described ADCs comprises a human immunoglobulin G subtype 1 (IgG1) heavy chain constant domain with a human Ig kappa light chain constant domain.

In some embodiments, the anti-B7-H3 (CD276) antibody or antigen-binding fragment thereof comprises three heavy chain CDRs and three light chain CDRs as follows: heavy chain CDR1 (HCDR1) consisting of SEQ ID NO:33, heavy chain CDR2 (HCDR2) consisting of SEQ ID NO:34, heavy chain CDR3 (HCDR3) consisting of SEQ ID NO:35; light chain CDR1 (LCDR1) consisting of SEQ ID NO:42, light chain CDR2 (LCDR2) consisting of SEQ ID NO:43, and light chain CDR3 (LCDR3) consisting of SEQ ID NO:44.

In some embodiments, the anti-B7-H3 (CD276) antibody or antigen-binding fragment thereof comprises three heavy chain CDRs and three light chain CDRs as follows: heavy chain CDR1 (HCDR1) consisting of SEQ ID NO:36, heavy chain CDR2 (HCDR2) consisting of SEQ ID NO:37, heavy chain CDR3 (HCDR3) consisting of SEQ ID NO:35; light chain CDR1 (LCDR1) consisting of SEQ ID NO:45, light chain CDR2 (LCDR2) consisting of SEQ ID NO:46, and light chain CDR3 (LCDR3) consisting of SEQ ID NO:47.

In some embodiments, the anti-B7-H3 (CD276) antibody or antigen-binding fragment thereof comprises three heavy chain CDRs and three light chain CDRs as follows: heavy chain CDR1 (HCDR1) consisting of SEQ ID NO:38, heavy chain CDR2 (HCDR2) consisting of SEQ ID NO:39, heavy chain CDR3 (HCDR3) consisting of SEQ ID NO:40; light chain CDR1 (LCDR1) consisting of SEQ ID NO:48, light chain CDR2 (LCDR2) consisting of SEQ ID NO:43, and light chain CDR3 (LCDR3) consisting of SEQ ID NO:47.

In some embodiments, the anti-B7-H3 (CD276) antibody or antigen-binding fragment thereof comprises three heavy chain CDRs and three light chain CDRs as follows: heavy chain CDR1 (HCDR1) consisting of SEQ ID NO:41, heavy chain CDR2 (HCDR2) consisting of SEQ ID NO:37, heavy chain CDR3 (HCDR3) consisting of SEQ ID NO:35; light chain CDR1 (LCDR1) consisting of SEQ ID NO:45, light chain CDR2 (LCDR2) consisting of SEQ ID NO:46, and light chain CDR3 (LCDR3) consisting of SEQ ID NO:47.

In some embodiments, the anti-B7-H3 (CD276) antibody or antigen-binding fragment thereof comprises the three heavy chain CDRs and three light chain CDRs, wherein the CDRs include no more than one, two, three, four, five, or six amino acid additions, deletions or substitutions of HCDR1 (SEQ ID NO:33), HCDR2 (SEQ ID NO:34), HCDR3 (SEQ ID NO:35); LCDR1 (SEQ ID NO:42), LCDR2 (SEQ ID NO:43), and LCDR3 (SEQ ID NO:44).

In some embodiments, the anti-B7-H3 (CD276) antibody or antigen-binding fragment thereof comprises the three heavy chain CDRs and three light chain CDRs, wherein the CDRs include no more than one, two, three, four, five, or six amino acid additions, deletions or substitutions of HCDR1 (SEQ ID NO:36), HCDR2 (SEQ ID NO:37), HCDR3 (SEQ ID NO:35); LCDR1 (SEQ ID NO:45), LCDR2 (SEQ ID NO:46), and LCDR3 (SEQ ID NO:47).

In some embodiments, the anti-B7-H3 (CD276) antibody or antigen-binding fragment thereof comprises the three heavy chain CDRs and three light chain CDRs, wherein the CDRs include no more than one, two, three, four, five, or six amino acid additions, deletions or substitutions of HCDR1 (SEQ ID NO:38), HCDR2 (SEQ ID NO:39), HCDR3 (SEQ ID NO:40); LCDR1 (SEQ ID NO:48), LCDR2 (SEQ ID NO:43), and LCDR3 (SEQ ID NO:47).

In some embodiments, the anti-B7-H3 (CD276) antibody or antigen-binding fragment thereof comprises the three heavy chain CDRs and three light chain CDRs, wherein the CDRs include no more than one, two, three, four, five, or six amino acid additions, deletions or substitutions of HCDR1 (SEQ ID NO:41), HCDR2 (SEQ ID NO:37), HCDR3 (SEQ ID NO:35); LCDR1 (SEQ ID NO:45), LCDR2 (SEQ ID NO:46), and LCDR3 (SEQ ID NO:47).

In some embodiments, the anti-B7-H3 (CD276) antibody or antigen-binding fragment thereof comprises three heavy chain CDRs and three light chain CDRs as follows: heavy chain CDR1 (HCDR1) consisting of SEQ ID NO:49, heavy chain CDR2 (HCDR2) consisting of SEQ ID NO:50, heavy chain CDR3 (HCDR3) consisting of SEQ ID NO:51; light chain CDR1 (LCDR1) consisting of SEQ ID NO:58, light chain CDR2 (LCDR2) consisting of SEQ ID NO:59, and light chain CDR3 (LCDR3) consisting of SEQ ID NO:60.

In some embodiments, the anti-B7-H3 (CD276) antibody or antigen-binding fragment thereof comprises three heavy chain CDRs and three light chain CDRs as follows: heavy chain CDR1 (HCDR1) consisting of SEQ ID NO:52, heavy chain CDR2 (HCDR2) consisting of SEQ ID NO:53, heavy chain CDR3 (HCDR3) consisting of SEQ ID NO:51; light chain CDR1 (LCDR1) consisting of SEQ ID NO:61 light chain CDR2 (LCDR2) consisting of SEQ ID NO:62, and light chain CDR3 (LCDR3) consisting of SEQ ID NO:63.

In some embodiments, the anti-B7-H3 (CD276) antibody or antigen-binding fragment thereof comprises three heavy chain CDRs and three light chain CDRs as follows: heavy chain CDR1 (HCDR1) consisting of SEQ ID NO:54, heavy chain CDR2 (HCDR2) consisting of SEQ ID NO:55, heavy chain CDR3 (HCDR3) consisting of SEQ ID NO:56; light chain CDR1 (LCDR1) consisting of SEQ ID NO:58, light chain CDR2 (LCDR2) consisting of SEQ ID NO:59, and light chain CDR3 (LCDR3) consisting of SEQ ID NO:63.

In some embodiments, the anti-B7-H3 (CD276) antibody or antigen-binding fragment thereof comprises three heavy chain CDRs and three light chain CDRs as follows: heavy chain CDR1 (HCDR1) consisting of SEQ ID NO:57, heavy chain CDR2 (HCDR2) consisting of SEQ ID NO:53, heavy chain CDR3 (HCDR3) consisting of SEQ ID NO:51; light chain CDR1 (LCDR1) consisting of SEQ ID NO:61, light chain CDR2 (LCDR2) consisting of SEQ ID NO:62, and light chain CDR3 (LCDR3) consisting of SEQ ID NO:63.

In some embodiments, the anti-B7-H3 (CD276) antibody or antigen-binding fragment thereof comprises the three heavy chain CDRs and three light chain CDRs, wherein the CDRs include no more than one, two, three, four, five, or six amino acid additions, deletions or substitutions of HCDR1 (SEQ ID NO:49), HCDR2 (SEQ ID NO:50), HCDR3 (SEQ ID NO:51); LCDR1 (SEQ ID NO:58), LCDR2 (SEQ ID NO:59), and LCDR3 (SEQ ID NO:60).

In some embodiments, the anti-B7-H3 (CD276) antibody or antigen-binding fragment thereof comprises the three heavy chain CDRs and three light chain CDRs, wherein the CDRs include no more than one, two, three, four, five, or six amino acid additions, deletions or substitutions of HCDR1 (SEQ ID NO:52), HCDR2 (SEQ ID NO:53), HCDR3 (SEQ ID NO:51); LCDR1 (SEQ ID NO:61), LCDR2 (SEQ ID NO:62), and LCDR3 (SEQ ID NO:63).

In some embodiments, the anti-B7-H3 (CD276) antibody or antigen-binding fragment thereof comprises the three heavy chain CDRs and three light chain CDRs, wherein the CDRs include no more than one, two, three, four, five, or six amino acid additions, deletions or substitutions of HCDR1 (SEQ ID NO:54), HCDR2 (SEQ ID NO:55), HCDR3 (SEQ ID NO:56); LCDR1 (SEQ ID NO:58), LCDR2 (SEQ ID NO:59), and LCDR3 (SEQ ID NO:63).

In some embodiments, the anti-B7-H3 (CD276) antibody or antigen-binding fragment thereof comprises the three heavy chain CDRs and three light chain CDRs, wherein the CDRs include no more than one, two, three, four, five, or six amino acid additions, deletions or substitutions of HCDR1 (SEQ ID NO:57), HCDR2 (SEQ ID NO:53), HCDR3 (SEQ ID NO:51); LCDR1 (SEQ ID NO:61), LCDR2 (SEQ ID NO:62), and LCDR3 (SEQ ID NO:63).

In some embodiments, the anti-B7-H3 (CD276) antibody or antigen-binding fragment thereof comprises a heavy chain variable region comprising the amino acid sequence of SEQ ID NO:13, and a light chain variable region comprising the amino acid sequence of SEQ ID NO:14. In some embodiments, the anti-B7-H3 (CD276) antibody or antigen-binding fragment thereof comprises the heavy chain variable region amino acid sequence of SEQ ID NO:13 and the light chain variable region amino acid sequence of SEQ ID NO:14, or sequences that are at least 95% identical to the disclosed sequences. In some embodiments, the anti-B7-H3 (CD276) antibody or antigen-binding fragment thereof has a heavy chain variable region amino acid sequence that is at least 96%, at least 97%, at least 98%, or at least 99% identical to SEQ ID NO:13 and/or a light chain variable region amino acid sequence that is at least 96%, at least 97%, at least 98%, or at least 99% identical to SEQ ID NO:14.

In some embodiments, the anti-B7-H3 (CD276) antibody or antigen-binding fragment thereof comprises a heavy chain variable region comprising the amino acid sequence of SEQ ID NO:15, and a light chain variable region comprising the amino acid sequence of SEQ ID NO:16. In some embodiments, the anti-B7-H3 (CD276) antibody or antigen-binding fragment thereof comprises the heavy chain variable region amino acid sequence of SEQ ID NO:15 and the light chain variable region amino acid sequence of SEQ ID NO:16, or sequences that are at least 95% identical to the disclosed sequences. In some embodiments, the anti-B7-H3 (CD276) antibody or antigen-binding fragment thereof has a heavy chain variable region amino acid sequence that is at least 96%, at least 97%, at least 98%, or at least 99% identical to SEQ ID NO:15 and/or a light chain variable region amino acid sequence that is at least 96%, at least 97%, at least 98%, or at least 99% identical to SEQ ID NO:16.

In some embodiments, the anti-B7-H3 (CD276) antibody comprises the heavy chain amino acid sequence of SEQ ID NO:7 or a sequence that is at least 95% identical to SEQ ID NO:7, and the light chain amino acid sequence of SEQ ID NO:8 or a sequence that is at least 95% identical to SEQ ID NO:8. In some embodiments, the anti-B7-H3 (CD276) antibody comprises the heavy chain amino acid sequence of SEQ ID NO:7 and the light chain amino acid sequence of SEQ ID NO:8, or sequences that are at least 95% identical to the disclosed sequences. In some embodiments, the anti-B7-H3 (CD276) antibody has a heavy chain amino acid sequence that is at least 96%, at least 97%, at least 98%, or at least 99% identical to SEQ ID NO:7 and a light chain amino acid sequence that is at least 96%, at least 97%, at least 98%, or at least 99% identical to SEQ ID NO:8.

In some embodiments, the anti-B7-H3 (CD276) antibody comprises a heavy chain amino acid sequence of SEQ ID NO:9 or a sequence that is at least 95% identical to SEQ ID NO:9, and the light chain amino acid sequence of SEQ ID NO:10 or a sequence that is at least 95% identical to SEQ ID NO:10. In some embodiments, the anti-B7-H3 (CD276) antibody comprises the heavy chain amino acid sequence of SEQ ID NO:9 and the light chain amino acid sequence of SEQ ID NO:10, or sequences that are at least 95% identical to the disclosed sequences. In some embodiments, the anti-B7-H3 (CD276) antibody has a heavy chain amino acid sequence that is at least 96%, at least 97%, at least 98%, or at least 99% identical to SEQ ID NO:9 and a light chain amino acid sequence that is at least 96%, at least 97%, at least 98%, or at least 99% identical to SEQ ID NO:10.

Residues in two or more polypeptides are said to “correspond” if the residues occupy an analogous position in the polypeptide structures. Analogous positions in two or more polypeptides can be determined by aligning the polypeptide sequences based on amino acid sequence or structural similarities. Those skilled in the art understand that it may be necessary to introduce gaps in either sequence to produce a satisfactory alignment.

In some embodiments, amino acid substitutions are of single residues. Insertions usually will be on the order of from about 1 to about 20 amino acid residues, although considerably larger insertions may be tolerated as long as biological function is retained (e.g., binding to a target antigen). Deletions usually range from about 1 to about 20 amino acid residues, although in some cases deletions may be much larger. Substitutions, deletions, insertions, or any combination thereof may be used to arrive at a final derivative or variant. Generally, these changes are done on a few amino acids to minimize the alteration of the molecule, particularly the immunogenicity and specificity of the antigen binding protein. However, larger changes may be tolerated in certain circumstances. Conservative substitutions can be made in accordance with the following chart depicted as Table 7.

TABLE 7

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

In some embodiments where variant antibody sequences are used in an ADC, the variants typically exhibit the same qualitative biological activity and will elicit the same immune response, although variants may also be selected to modify the characteristics of the antigen binding proteins as needed. Alternatively, the variant may be designed such that the biological activity of the antigen binding protein is altered. For example, glycosylation sites may be altered or removed.

Various antibodies may be used with the ADCs used herein to target cancer cells. As shown below, the linker-payloads in the ADCs disclosed herein are surprisingly effective with different tumor antigen-targeting antibodies. Suitable antigens expressed on cancer cells but not healthy cells, or expressed on cancer cells at a higher level than on healthy cells, are known in the art, as are antibodies directed against them. Further antibodies against those antigen targets may be prepared by those of skill in the art. These antibodies may be used with the conjugate linkers and panRAS inhibitor payloads disclosed herein. In some embodiments, the antibody or antigen-binding fragment targets EphA2 or B7-H3 (CD276) provided particularly improved drug:antibody ratio, aggregation level, stability (i.e., in vitro and in vivo stability), tumor targeting (i.e., cytotoxicity, potency), minimized off-target killing, and/or treatment efficacy. Improved treatment efficacy can be measured in vitro or in vivo, and may include reduced tumor growth rate and/or reduced tumor volume.

In some embodiments, alternate antibodies to the same targets or antibodies to different antigen targets are used and provide at least some of the favorable functional properties described above (e.g., improved stability, improved tumor targeting, improved treatment efficacy, etc.). In some embodiments, some or all of these favorable functional properties are observed when the disclosed conjugate linkers and panRAS inhibitor payloads are conjugated to an alternate EphA2 or B7-H3 (CD276) targeting antibody or antigen-binding fragment. In some other embodiments, some or all of these favorable functional properties are observed when the disclosed conjugate linkers and panRAS inhibitor payloads are conjugated to a EphA2-targeting antibody or antigen-binding fragment. In some embodiments, the antibody or antigen-binding fragment targets EphA2. In other embodiments, some or all of these favorable functional properties are observed when the disclosed conjugate linkers and panRAS inhibitor payloads are conjugated to a B7-H3 (CD276)-targeting antibody or antigen-binding fragment. In some embodiments, the antibody or antigen-binding fragment targets B7-H3 (CD276).

### Conjugate Linkers

In some embodiments, the conjugate linker in an ADC is stable extracellularly in a sufficient manner to be therapeutically effective. In some embodiments, the conjugate linker is stable outside a cell, such that the ADC remains intact when present in extracellular conditions (e.g., prior to transport or delivery into a cell). The term “intact,” used in the context of an ADC, means that the antibody or antigen-binding fragment remains attached to the drug moiety (e.g., the panRAS inhibitor).

As used herein, “stable,” in the context of a conjugate linker or ADC comprising a conjugate linker, means that no more than 20%, no more than about 15%, no more than about 10%, no more than about 5%, no more than about 3%, or no more than about 1% of the conjugate linkers (or any percentage in between) in a sample of ADC are cleaved (or in the case of an overall ADC are otherwise not intact) when the ADC is present in extracellular conditions. In some embodiments, the conjugate linkers and/or ADCs disclosed herein are stable compared to alternate conjugate linkers and/or ADCs with alternate conjugate linkers and/or panRAS inhibitor payloads. In some embodiments, the ADCs disclosed herein can remain intact for more than about 48 hours, more than 60 hours, more than about 72 hours, more than about 84 hours, or more than about 96 hours.

Whether a conjugate linker is stable extracellularly can be determined, for example, by including an ADC in plasma for a predetermined time period (e.g., 2, 4, 6, 8, 16, 24, 48, or 72 hours) and then quantifying the amount of free drug moiety present in the plasma. Stability may allow the ADC time to localize to target cancer cells and prevent the premature release of the drug moiety, which could lower the therapeutic index of the ADC by indiscriminately damaging both normal and cancer tissues. In some embodiments, the conjugate linker is stable outside of a target cell and releases the drug moiety from the ADC once inside of the cell, such that the drug can bind to its target. Thus, an effective conjugate linker will: (i) maintain the specific binding properties of the antibody or antigen-binding fragment; (ii) allow delivery, e.g., intracellular delivery, of the drug moiety via stable attachment to the antibody or antigen-binding fragment; (iii) remain stable and intact until the ADC has been transported or delivered to its target site; and (iv) allow for the therapeutic effect, e.g., cytotoxic effect, of the drug moiety after cleavage or alternate release mechanism.

Conjugate linkers may impact the physico-chemical properties of an ADC. As many cytotoxic agents are hydrophobic in nature, linking them to the antibody with an additional hydrophobic moiety may lead to aggregation. ADC aggregates are insoluble and often limit achievable drug loading onto the antibody, which can negatively affect the potency of the ADC. Protein aggregates of biologics, in general, have also been linked to increased immunogenicity. As shown below, conjugate linkers disclosed herein result in ADCs with low aggregation levels and desirable levels of drug loading.

A conjugate linker may be “cleavable” or “non-cleavable” (Ducry and Stump (2010) Bioconjugate Chem. 21:5-13). Cleavable conjugate linkers are designed to release the drug moiety (e.g., a panRAS inhibitor) when subjected to certain environment factors, e.g., when internalized into the target cell, whereas non-cleavable conjugate linkers generally rely on the degradation of the antibody or antigen-binding fragment itself.

The term “alkyl”, as used herein, refers to a straight or branched hydrocarbon chain radical consisting solely of carbon and hydrogen atoms, containing no unsaturation. The term “C₁-C₆alkyl”, as used herein, refers to a straight or branched hydrocarbon chain radical consisting solely of carbon and hydrogen atoms, containing no unsaturation, having from one to six carbon atoms, and which is attached to the rest of the molecule by a single bond. Non-limiting examples of “C₁-C₆alkyl” groups include methyl (a C₁alkyl), ethyl (a C₂alkyl), 1-methylethyl (a C₃alkyl), n-propyl (a C₃alkyl), isopropyl (a C₃alkyl), n-butyl (a C₄alkyl), isobutyl (a C₄alkyl), sec-butyl (a C₄alkyl), tert-butyl (a C₄alkyl), n-pentyl (a C₅alkyl), isopentyl (a C₅alkyl), neopentyl (a C₅alkyl) and hexyl (a C₆alkyl).

The term “alkenyl”, as used herein, refers to a straight or branched hydrocarbon chain radical group consisting solely of carbon and hydrogen atoms, containing at least one double bond. The term “C₂-C₆alkenyl”, as used herein, refers to a straight or branched hydrocarbon chain radical group consisting solely of carbon and hydrogen atoms, containing at least one double bond, having from two to six carbon atoms, which is attached to the rest of the molecule by a single bond. Non-limiting examples of “C₂-C₆alkenyl” groups include ethenyl (a C₂alkenyl), prop-1-enyl (a C₃alkenyl), but-1-enyl (a C₄alkenyl), pent-1-enyl (a C₅alkenyl), pent-4-enyl (a C₅alkenyl), penta-1,4-dienyl (a C₅alkenyl), hexa-1-enyl (a C₆alkenyl), hexa-2-enyl (a C₆alkenyl), hexa-3-enyl (a C₆alkenyl), hexa-1-,4-dienyl (a C₆alkenyl), hexa-1-,5-dienyl (a C₆alkenyl) and hexa-2-,4-dienyl (a C₆alkenyl). The term “C₂-C₃alkenyl”, as used herein, refers to a straight or branched hydrocarbon chain radical group consisting solely of carbon and hydrogen atoms, containing at least one double bond, having from two to three carbon atoms, which is attached to the rest of the molecule by a single bond. Non-limiting examples of “C₂-C₃alkenyl” groups include ethenyl (a C₂alkenyl) and prop-1-enyl (a C₃alkenyl).

The term “alkylene”, as used herein, refers to a bivalent straight or branched hydrocarbon chain radical consisting solely of carbon and hydrogen atoms and containing no unsaturation. The term “C₁-C₆alkylene”, as used herein, refers to a bivalent straight or branched hydrocarbon chain radical consisting solely of carbon and hydrogen atoms, containing no unsaturation, having from one to six carbon atoms. Non-limiting examples of “C₁-C₆alkylene” groups include methylene (a C₁alkylene), ethylene (a C₂alkylene), 1-methylethylene (a C₃alkylene), n-propylene (a C₃alkylene), isopropylene (a C₃alkylene), n-butylene (a C₄alkylene), isobutylene (a C₄alkylene), sec-butylene (a C₄alkylene), tert-butylene (a C₄alkylene), n-pentylene (a C₅alkylene), isopentylene (a C₅alkylene), neopentylene (a C₅alkylene), and hexylene (a C₅alkylene).

The term “alkenylene”, as used herein, refers to a bivalent straight or branched hydrocarbon chain radical consisting solely of carbon and hydrogen atoms and containing at least one double bond. The term “C₂-C₆alkenylene”, as used herein, refers to a bivalent straight or branched hydrocarbon chain radical group consisting solely of carbon and hydrogen atoms, containing at least one double bond, and having from two to six carbon atoms. Non-limiting examples of “C₂-C₆alkenylene” groups include ethenylene (a C₂alkenylene), prop-1-enylene (a C₃alkenylene), but-1-enylene (a C₄alkenylene), pent-1-enylene (a C₅alkenylene), pent-4-enylene (a C₅alkenylene), penta-1,4-dienylene (a C₅alkenylene), hexa-1-enylene (a C₆alkenylene), hexa-2-enylene (a C₆alkenylene), hexa-3-enylene (a C₆alkenylene), hexa-1-,4-dienylene (a C₆alkenylene), hexa-1-,5-dienylene (a C₆alkenylene) and hexa-2-,4-dienylene (a C₆alkenylene). The term “C₂-C₆alkenylene”, as used herein, refers to a bivalent straight or branched hydrocarbon chain radical group consisting solely of carbon and hydrogen atoms, containing at least one double bond, and having from two to three carbon atoms. Non-limiting examples of “C₂-C₃alkenylene” groups include ethenylene (a C₂alkenylene) and prop-1-enylene (a C₃alkenylene).

The term “cycloalkyl,” as used herein, refers to a non-aromatic, monocyclic, fused bicyclic, fused tricyclic or bridged polycyclic ring system. In some embodiments, the cycloalkyl is a mono- or bi-cyclic saturated carbocyclic group containing from 3 to 10 ring members, which may include fused, bridged or spiro ring systems. Non-limiting examples of fused bicyclic or bridged polycyclic ring systems include bicyclo[1.1.1]pentane, bicyclo[2.1.1]hexane, bicyclo[2.2.1]heptane, bicyclo[3.1.1]heptane, bicyclo[3.2.1]octane, bicyclo[2.2.2]octane and adamantanyl. Non-limiting examples monocyclic C₃-C₈cycloalkyl groups include cyclopropyl, cyclobutyl, cyclopentyl, cyclohexyl, cycloheptyl and cyclooctyl groups.

The term heteroarylene, cycloalkylene, heterocycloalkylene mean a divalent heteroaryl, cycloalkyl and heterocycloalkyl.

The term “haloalkyl,” as used herein, refers to a linear or branched alkyl chain substituted with one or more halogen groups in place of hydrogens along the hydrocarbon chain. Examples of halogen groups suitable for substitution in the haloalkyl group include Fluorine, Bromine, Chlorine, and Iodine. Haloalkyl groups may include substitution with multiple halogen groups in place of hydrogens in an alkyl chain, wherein said halogen groups can be attached to the same carbon or to another carbon in the alkyl chain.

As used herein, the alkyl, alkenyl, alkynyl, alkoxy, amino, aryl, heteroaryl, cycloalkyl, and heterocycloalkyl groups may be optionally substituted by 1 to 4 groups selected from optionally substituted linear or branched (C₁-C₆)alkyl, optionally substituted linear or branched (C₂-C₆)alkenyl group, optionally substituted linear or branched (C₂-C₆)alkynyl group, optionally substituted linear or branched (C₁-C₆)alkoxy, optionally substituted (C₁-C₆)alkyl-S—, hydroxy, oxo (or N-oxide where appropriate), nitro, cyano, —C(O)—OR₀′, —O—C(O)—R₀′, —C(O)—NR₀′R₀″, —NR₀′R₀″, —(C═NR₀′)—OR₀″, linear or branched (C₁-C₆) haloalkyl, trifluoromethoxy, or halogen, wherein R₀′ and R₀″ are each independently a hydrogen atom or an optionally substituted linear or branched (C₁-C₆)alkyl group, and wherein one or more of the carbon atoms of linear or branched (C₁-C₆)alkyl group is optionally deuterated.

The term “polyoxyethylene”, “polyethylene glycol” or “PEG”, as used herein, refers to a linear chain, a branched chain or a star shaped configuration comprised of (OCH₂CH₂) groups. In certain embodiments a polyethylene or PEG group is —(OCH₂CH₂)ₜ*—, where t is 1-40 or 4-40, and where the “-” indicates the end directed toward the self-immolative spacer and the “*-” indicates the point of attachment to a terminal end group R′ where R′ is OH, OCH₃ or OCH₂CH₂C(═O)OH. In other embodiments a polyethylene or PEG group is —(CH₂CH₂O)ₜ*—, where t is 1-40 or 4-40, and where the “-” indicates the end directed toward the self-immolative spacer and the “*-” indicates the point of attachment to a terminal end group R″ where R″ is H, CH₃ or CH₂CH₂C(═O)OH. For example, the term “PEG12” as used herein means that t is 12.

The term “polyalkylene glycol”, as used herein, refers to a linear chain, a branched chain or a star shaped configuration comprised of (O(CH₂)ₘ)ₙ groups. In certain embodiments a polyethylene or PEG group is —(O(CH₂)ₘ)ₜ*—, where m is 1-10, t is 1-40 or 4-40, and where the “-” indicates the end directed toward the self-immolative spacer and the “*-” indicates the point of attachment to a terminal end group R′ where R′ is OH, OCH₃ or OCH₂CH₂C(═O)OH. In other embodiments a polyethylene or PEG group is —((CH₂)ₘO)ₜ*—, where m is 1-10, t is 1-40 or 4-40, and where the “-” indicates the end directed toward the self-immolative spacer and the “*-” indicates the point of attachment to a terminal end group R″ where R″ is H, CH₃ or CH₂CH₂C(═O)OH.

The term “reactive group”, as used herein, is a functional group capable of forming a covalent bond with a functional group of an antibody, an antibody fragment, or another reactive group attached to an antibody or antibody fragment. Non limiting examples of such functional groups include reactive groups of Table 8 provided herein.

The term “attachment group” or “coupling group”, as used herein, refers to a bivalent moiety which links the bridging spacer to the antibody or fragment thereof. The attachment or coupling group is a bivalent moiety formed by the reaction between a reaction group and a functional group on the antibody or fragment thereof. Non limiting examples of such bivalent moieties include the bivalent chemical moieties given in Table 8 and Table 9 provided herein.

The term “bridging spacer”, as used herein, refers to one or more conjugate linker components which are covalently attached together to form a bivalent moiety which links the bivalent peptide spacer to the reactive group, links the bivalent peptide space to the coupling group, or links the attachment group to the at least one cleavable group. In certain embodiments the “bridging spacer” comprises a carboxyl group attached to the N-terminus of the bivalent peptide spacer via an amide bond.

The term “spacer moiety”, as used herein, refers to one or more conjugate linker components which are covalently attached together to form a moiety which links the self-immolative spacer to the hydrophilic moiety.

The term “bivalent peptide spacer”, as used herein, refers to bivalent conjugate linker comprising one or more amino acid residues covalently attached together to form a moiety which links the bridging spacer to the self immolative spacer. The one or more amino acid residues can be an residue of amino acids selected from alanine (Ala), cysteine (Cys), aspartic acid (Asp), glutamic acid (Glu), phenylalanine (Phe), glycine (Gly), histidine (His), isoleucine (lie), lysine (Lys), leucine (Leu), methionine (Met), asparagine (Asn), proline (Pro), glutamine (Gln), arginine (Arg), serine (Ser), threonine (Thr), valine (Val), tryptophan (Trp), tyrosine (Tyr), citrulline (Cit), norvaline (Nva), norleucune (Nle), selenocysteine (Sec), pyrrolysine (Pyl), homoserine, homocysteine, and desmethyl pyrrolysine.

In certain embodiments a “bivalent peptide spacer” is a combination of 2 to four amino acid residues where each residue is independently selected from a residue of an amino acid selected from alanine (Ala), cysteine (Cys), aspartic acid (Asp), glutamic acid (Glu), phenylalanine (Phe), glycine (Gly), histidine (His), isoleucine (lie), lysine (Lys), leucine (Leu), methionine (Met), asparagine (Asn), proline (Pro), glutamine (Gln), arginine (Arg), serine (Ser), threonine (Thr), valine (Val), tryptophan (Trp), tyrosine (Tyr), citrulline (Cit), norvaline (Nva), norleucune (Nle), selenocysteine (Sec), pyrrolysine (Pyl), homoserine, homocysteine, and desmethyl pyrrolysine, for example -ValCit*; -CitVal*; -AlaAla*; -AlaCit*; -CitAla*; -AsnCit*; -CitAsn*; -CitCit*; -ValGlu*; -GluVal*; -SerCit*; -CitSer*; -LysCit*; -CitLys*; -AspCit*; -CitAsp*; -AlaVal*; -ValAla*; -PheAla*; -AlaPhe*; -PheLys*; -LysPhe*; -ValLys*; -LysVal*; -AlaLys*; -LysAla*; -PheCit*; -CitPhe*; -LeuCit*; -CitLeu*; -IleCit*; -CitIle*; -PheArg*; -ArgPhe*; -CitTrp*; -TrpCit*; -PhePheLys*; -LysPhePhe*; -DPhePheLys*; -DLysPhePhe*; -GlyPheLys*; -LysPheGly*; -GlyPheLeuGly- [SEQ ID NO:67]; -GlyLeuPheGly- [SEQ ID NO:68]; -AlaLeuAlaLeu- [SEQ ID NO:69], -GlyGlyGly*; -GlyGlyGlyGly- [SEQ ID NO:70]; -GlyPheValGly-[SEQ ID NO:71]; and -GlyValPheGly- [SEQ ID NO:72], where the “-” indicates the point of attachment to the bridging spacer and the “*” indicates the point of attachment to the self-immolative spacer.

The term “conjugate linker component”, as used herein, refers to a chemical moiety that is a part of the conjugate linker. Examples of conjugate linker components include: an alkylene group: —(CH₂)ₙ— which can either be linear or branched (where in this instance n is 1-18); an alkenylene group; an alkynylene group; an alkenyl group; an alkynyl group; an ethylene glycol unit: —OCH₂CH₂— or —CH₂CH₂O—; an polyethylene glycol unit: (—CH₂CH₂O-)ₓ (where x in this instance is 2-20); —O—; —S—; a carbonyl: —C(═O); an ester: C(═O)—O or O—C(═O); a carbonate: —OC(═O)O—; an amine: —NH—; an tertiary amine; an amide: —C(═O)—NH—, —NH—C(═O)— or —C(═O)N(C₁₋₆alkyl); a carbamate: —OC(═O)NH— or —NHC(═O)O; a urea: —NHC(═O)NH; a sulfonamide: —S(O)₂NH— or —NHS(O)₂; an ether: —CH₂O— or —OCH₂—; an alkylene substituted with one or more groups independently selected from carboxy, sulfonate, hydroxyl, amine, amino acid, saccharide, phosphate and phosphonate); an alkenylene substituted with one or more groups independently selected from carboxy, sulfonate, hydroxyl, amine, amino acid, saccharide, phosphate and phosphonate); an alkynylene substituted with one or more groups independently selected from carboxy, sulfonate, hydroxyl, amine, amino acid, saccharide, phosphate and phosphonate); a C₁-C₁₀alkylene in which one or more methylene groups is replace by one or more —S—, —NH— or —O— moieties; a ring systems having two available points of attachment such as a divalent ring selected from phenyl (including 1,2-1,3- and 1,4-di-substituted phenyls), a C₅-C₆ heteroaryl, a C₃—C cycloalkyl (including 1,1-disubstituted cyclopropyl, cyclobutyl, cyclopentyl or cyclohexyl, and 1,4-disubstituted cyclohexyl), and a C₄-C₈ heterocycloalkyl; a residue of an amino acid selected from alanine (Ala), cysteine (Cys), aspartic acid (Asp), glutamic acid (Glu), phenylalanine (Phe), glycine (Gly), histidine (His), isoleucine (lie), lysine (Lys), leucine (Leu), methionine (Met), asparagine (Asn), proline (Pro), glutamine (Gln), arginine (Arg), serine (Ser), threonine (Thr), valine (Val), tryptophan (Trp), tyrosine (Tyr), citrulline (Cit), norvaline (Nva), norleucune (Nle), selenocysteine (Sec), pyrrolysine (Pyl), homoserine, homocysteine, and desmethyl pyrrolysine; a combination of 2 or more amino acid residues where each residue is independently selected from a residue of an amino acid selected from alanine (Ala), cysteine (Cys), aspartic acid (Asp), glutamic acid (Glu), phenylalanine (Phe), glycine (Gly), histidine (His), isoleucine (lie), lysine (Lys), leucine (Leu), methionine (Met), asparagine (Asn), proline (Pro), glutamine (Gln), arginine (Arg), serine (Ser), threonine (Thr), valine (Val), tryptophan (Trp), tyrosine (Tyr), citrulline (Cit), norvaline (Nva), norleucune (Nle), selenocysteine (Sec), pyrrolysine (Pyl), homoserine, homocysteine, and desmethyl pyrrolysine, for example Val-Cit; Cit-Val; Ala-Ala; Ala-Cit; Cit-Ala; Asn-Cit; Cit-Asn; Cit-Cit; Val-Glu; Glu-Val; Ser-Cit; Cit-Ser; Lys-Cit; Cit-Lys; Asp-Cit; Cit-Asp; Ala-Val; Val-Ala; Phe-Lys; Lys-Phe; Val-Lys; Lys-Val; Ala-Lys; Lys-Ala; Phe-Cit; Cit-Phe; Leu-Cit; Cit-Leu; Ile-Cit; Cit-Ile; Phe-Arg; Arg-Phe; Cit-Trp; and Trp-Cit; and a self-immolative spacer, wherein the self-immolative spacer comprises one or more protecting (triggering) groups which are susceptible to acid-induced cleavage, peptidase-induced cleavage, esterase-induced cleavage, glycosidase induced cleavage, phosphodiesterase induced cleavage, phosphatase induced cleavage, protease induced cleavage, lipase induced cleavage or disulfide bond cleavage.

Non-limiting examples of such self-immolative spacers include:

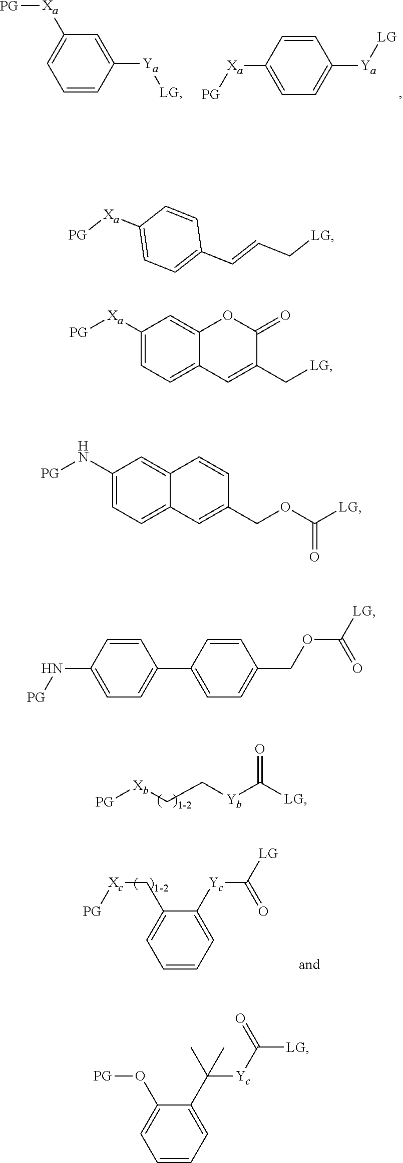

where:
PG is a protecting (triggering) group;
Xₐ is O, NH or S;
X_{b} is O, NH, NCH₃ or S;
X_{c} is O or NH;
Yₐ is CH₂, CH₂O or CH₂NH;
Y_{b} is CH₂, O or NH;
Y_{c} is a bond, CH₂, O or NH, and
LG is a leaving group such as a Drug moiety (D) of the Linker-Drug group of the invention.

Additional non-limiting examples of such self-immolative spacers are described in Angew. Chem. Int. Ed. 2015, 54, 7492-7509.

In addition, a conjugate linker component can be a chemical moiety which is readily formed by reaction between two reactive groups. Non-limiting examples of such chemical moieties are given in Table 8.

TABLE 8

| Reactive Group 1 | Reactive Group 2 | |
|---|---|---|
| (RG1) | (RG2) | Chemical Moiety |
| a thiol | a thiol | —S—S— |
| a thiol | a maleimide | 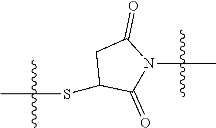 [SMILES: *SC1CC(=O)N(*)C1=O] |
| a thiol | a haloacetamide | 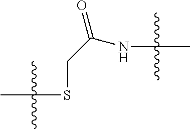 [SMILES: *NC(=O)CS*] |
| an azide | an alkyne | 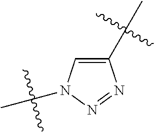 [SMILES: *c1cn(*)nn1] |
| | | or |
| | | 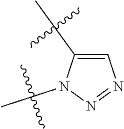 [SMILES: *c1cnnn1*] |
| an azide | a triaryl phosphine | 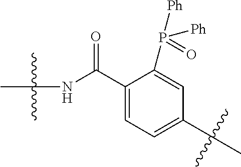 [SMILES: *NC(=O)c1ccc(*)cc1P(=O)(c1ccccc1)c1ccccc1] |
| an azide | a cyclooctyne | 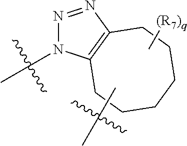 |
| | | or |
| | | 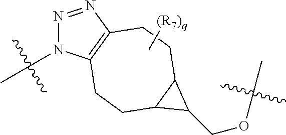 |
| | | or |
| | | 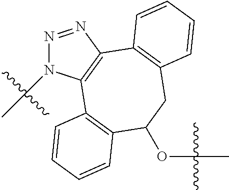 [SMILES: *OC1Cc2ccccc2-c2nnn(*)c2-c2ccccc21] |
| an azide | an oxanobornadiene | 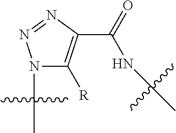 |
| a triaryl phosphine | an azide | 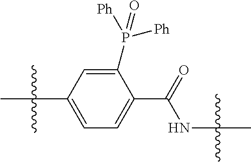 [SMILES: *NC(=O)c1ccc(*)cc1P(=O)(c1ccccc1)c1ccccc1] |
| an oxano- bornadiene | an azide | 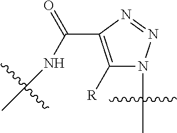 |
| an alkyne | an azide | 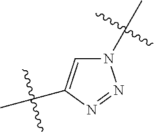 [SMILES: *c1cn(*)nn1] |
| | | or |
| | | 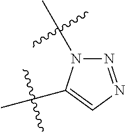 [SMILES: *c1cnnn1*] |
| a cyclooctyne | azide | 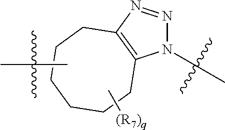 |
| | | or |
| | | 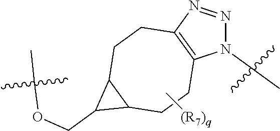 |
| | | or |
| | | 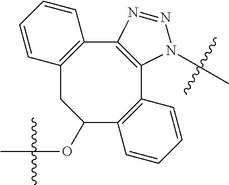 [SMILES: *OC1Cc2ccccc2-c2nnn(*)c2-c2ccccc21] |
| a cyclooctene | a diaryl tetrazine | 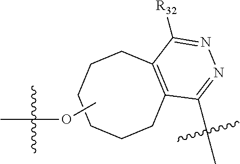 |
| | | or |
| | | 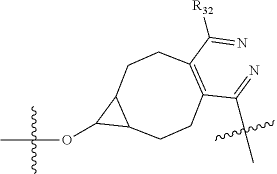 |
| a diaryl tetrazine | a cyclooctene | 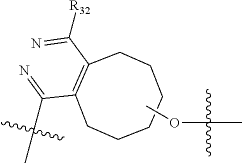 |
| | | or |
| | | 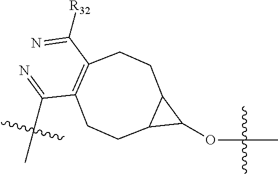 |
| a monoaryl tetrazine | a norbornene | 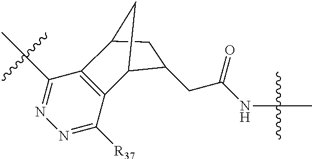 |
| a norbornene | a monoaryl tetrazine | 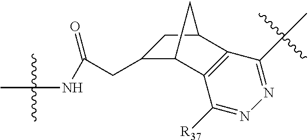 |
| an aldehyde | a hydroxylamine | 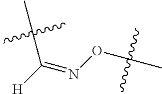 [SMILES: */C=N\O*] |
| an aldehyde | a hydrazine | 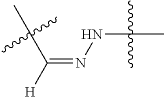 [SMILES: */C=N\N*] |
| an aldehyde | NH₂—NH— C(═O)— | 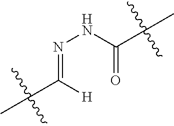 [SMILES: */C=N/NC(*)=O] |
| a ketone | a hydroxylamine | 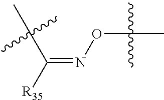 |
| a ketone | a hydrazine | 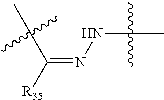 |
| a ketone | NH₂—NH— C(═O)— | 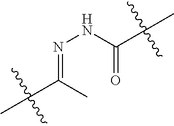 [SMILES: *C(=O)N/N=C(/*)C] |
| a hydroxylamine | an aldehyde | 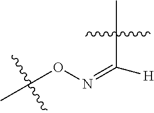 [SMILES: */C=N\O*] |
| a hydroxylamine | a ketone | 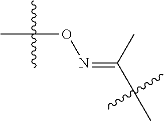 [SMILES: *O/N=C(/*)C] |
| a hydrazine | an aldehyde | 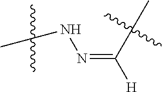 [SMILES: */C=N\N*] |
| a hydrazine | a ketone | 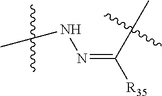 |
| NH₂—NH— C(═O)— | an aldehyde | 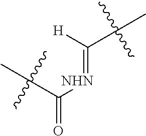 |
| NH₂—NH— C(═O)— | a ketone | 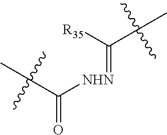 |
| a haloacetamide | a thiol | 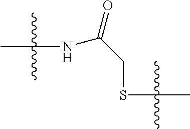 [SMILES: *NC(=O)CS*] |
| a maleimide | a thiol | 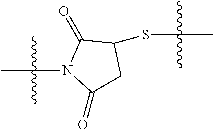 [SMILES: *SC1CC(=O)N(*)C1=O] |
| a vinyl sulfone | a thiol | 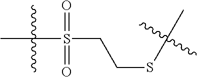 [SMILES: *SCCS(*)(=O)=O] |
| a thiol | a vinyl sulfone | 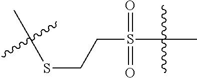 [SMILES: *SCCS(*)(=O)=O] |
| an aziridine | a thiol | 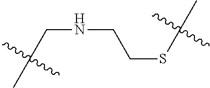 [SMILES: *CNCCS*] |
| | | or |
| | | 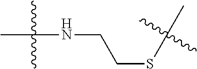 [SMILES: *NCCS*] |
| a thiol | an aziridine | 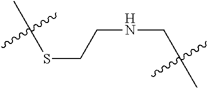 [SMILES: *CNCCS*] |
| | | or |
| | | 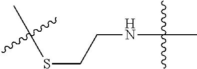 [SMILES: *NCCS*] |
| 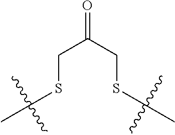 [SMILES: *SCC(=O)CS*] | hydroxylamine | 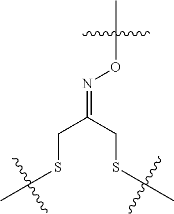 [SMILES: *ON=C(CS*)CS*] |
| 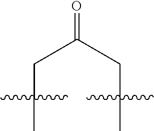 [SMILES: *CC(=O)C*] | hydroxylamine | 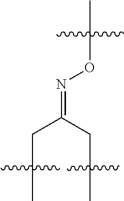 [SMILES: *CC(C*)=NO*] |
| 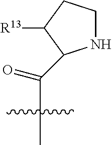 | 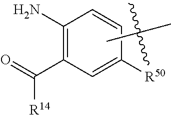 | 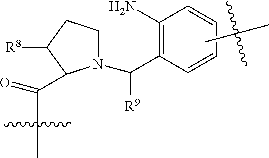 |
| 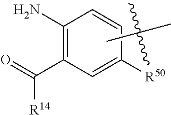 | 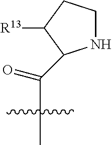 | 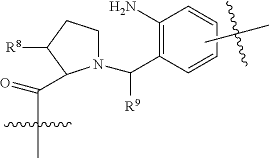 |
| 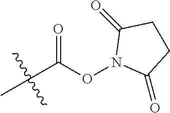 [SMILES: *C(=O)ON1C(=O)CCC1=O] | —NH₂, | amide |
| 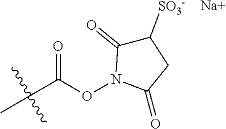 | | |
| 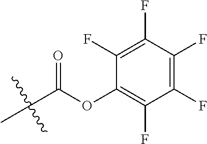 [SMILES: *C(=O)Oc1c(F)c(F)c(F)c(F)c1F] | | |
| 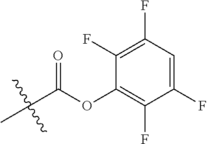 [SMILES: *C(=O)Oc1c(F)c(F)cc(F)c1F] | | |
| 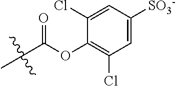 | | |
| —NH₂, | 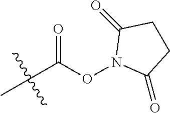 [SMILES: *C(=O)ON1C(=O)CCC1=O] | amide |
| | 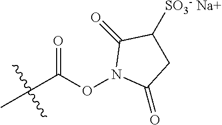 | |
| | 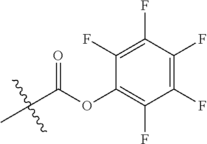 [SMILES: *C(=O)Oc1c(F)c(F)c(F)c(F)c1F] | |
| | 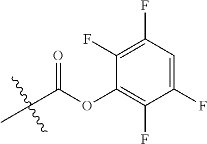 [SMILES: *C(=O)Oc1c(F)c(F)cc(F)c1F] | |
| | 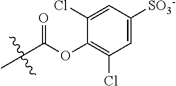 | |
| CoA or CoA analogue | Serine residue | 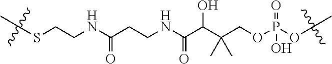 [SMILES: *OP(=O)(O)OCC(C)(C)C(O)C(=O)NCCC(=O)NCCS*] |
| | | 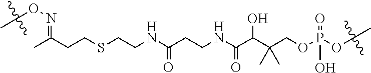 [SMILES: *O/N=C(\C)CCSCCNC(=O)CCNC(=O)C(O)C(C)(C)COP(=O)(O)O*] |
| | | 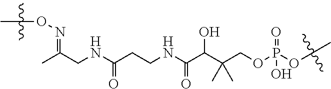 [SMILES: *O/N=C(\C)CNC(=O)CCNC(=O)C(O)C(C)(C)COP(=O)(O)O*] |
| | | 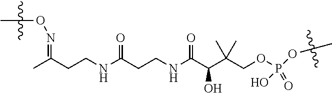 [SMILES: *O/N=C(\C)CCNC(=O)CCNC(=O)[C@H](O)C(C)(C)COP(=O)(O)O*] |
| | | 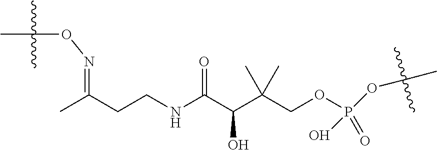 [SMILES: *O/N=C(\C)CCNC(=O)[C@H](O)C(C)(C)COP(=O)(O)O*] |
| | | 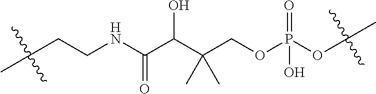 [SMILES: *CCNC(=O)C(O)C(C)(C)COP(=O)(O)O*] |
| | | 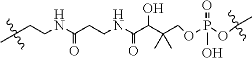 [SMILES: *CCNC(=O)CCNC(=O)C(O)C(C)(C)COP(=O)(O)O*] |
| | | 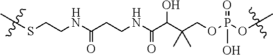 [SMILES: *OP(=O)(O)OCC(C)(C)C(O)C(=O)NCCC(=O)NCCS*] |
| pyridyldithiol | thiol | disulfide |

where: R³² in Table 8 is H, C₁₋₄ alkyl, phenyl, pyrimidine or pyridine; R³⁵ in Table 8 is H, C₁-C₆alkyl, phenyl or C₁₋₄alkyl substituted with 1 to 3 —OH groups; each R⁷ in Table 8 is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH; R³⁷ in Table 8 is independently selected from H, phenyl and pyridine; q in Table 8 is 0, 1, 2 or 3; R⁸ and R¹³ in Table 8 is H or methyl; and R⁹ and R¹⁴ in Table 8 is H, —CH₃ or phenyl; R in Table 8 is H or any suitable substituent; and R⁵⁰ in Table 8 is H.

In addition, a conjugate linker component can be a group listed in Table 9 below.

TABLE 9

| 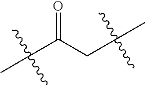 [SMILES: *CC(*)=O] | 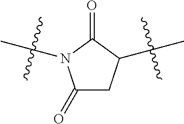 [SMILES: *C1CC(=O)N(*)C1=O] |
|---|---|
| 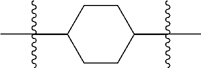 [SMILES: *C1CCC(*)CC1] | 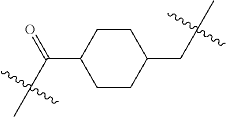 [SMILES: *CC1CCC(C(*)=O)CC1] |
| 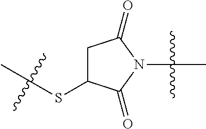 [SMILES: *SC1CC(=O)N(*)C1=O] | 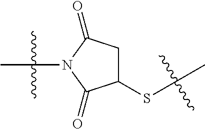 [SMILES: *SC1CC(=O)N(*)C1=O] |
| 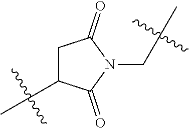 [SMILES: *CN1C(=O)CC(*)C1=O] | 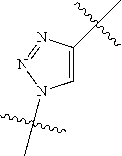 [SMILES: *c1cn(*)nn1] |
| 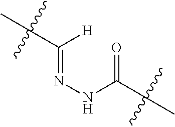 [SMILES: */C=N/NC(*)=O] | 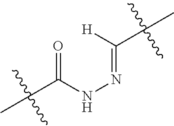 [SMILES: */C=N/NC(*)=O] |
| 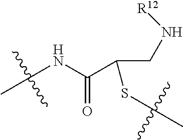 | 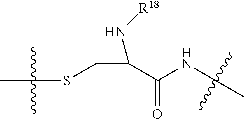 |
| 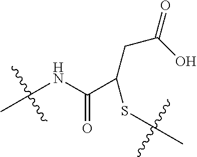 [SMILES: *NC(=O)C(CC(=O)O)S*] | 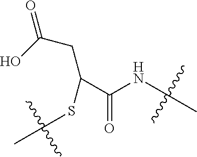 [SMILES: *NC(=O)C(CC(=O)O)S*] |
| 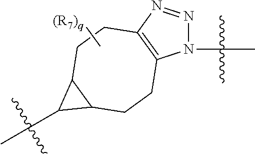 | 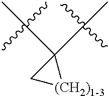 [SMILES: *C1(*)CC1] |
| 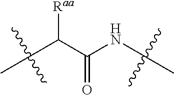 | 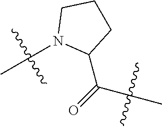 [SMILES: *C(=O)C1CCCN1*] |
| 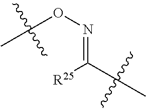 | 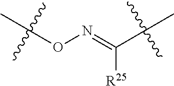 |
| 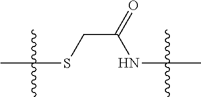 [SMILES: *NC(=O)CS*] | 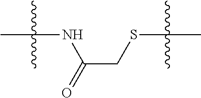 [SMILES: *NC(=O)CS*] |
| 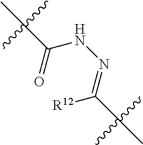 | 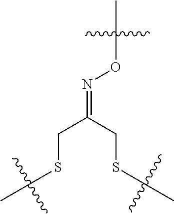 [SMILES: *ON=C(CS*)CS*] |
| 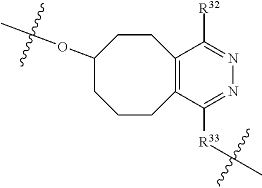 | 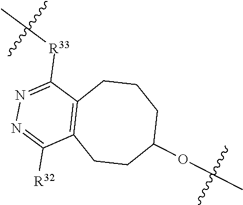 |
| 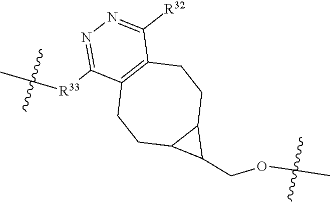 | 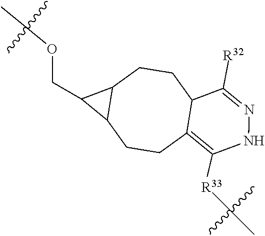 |
| 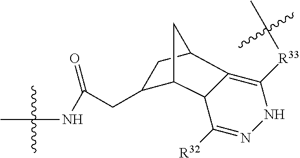 | 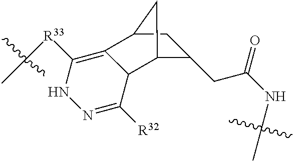 |
| 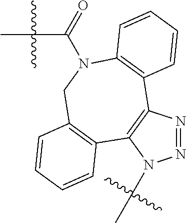 [SMILES: *C(=O)N1Cc2ccccc2-c2c(nnn2*)-c2ccccc21] | 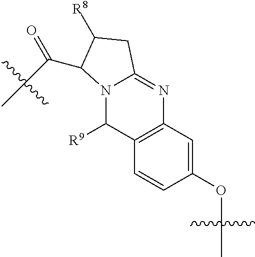 |
| 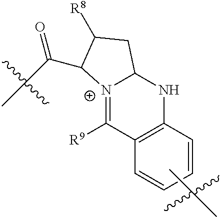 | 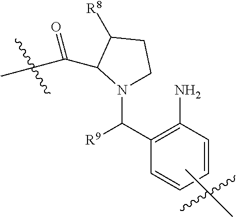 |
| 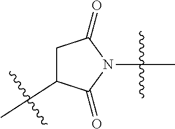 [SMILES: *C1CC(=O)N(*)C1=O] | 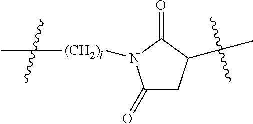 |
| 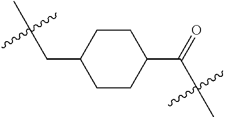 [SMILES: *CC1CCC(C(*)=O)CC1] | 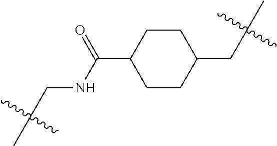 [SMILES: *CNC(=O)C1CCC(C*)CC1] |
| 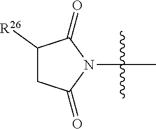 | 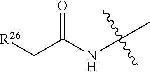 |
| 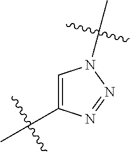 [SMILES: *c1cn(*)nn1] | 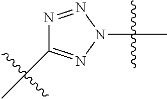 [SMILES: *c1nnn(*)n1] |
| 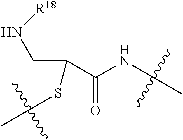 | 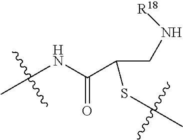 |
| 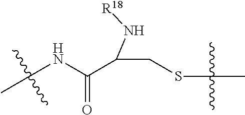 | 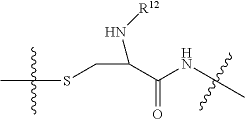 |
| 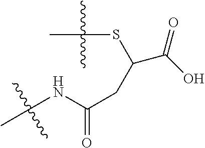 [SMILES: *NC(=O)CC(S*)C(=O)O] | 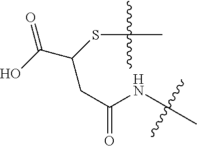 [SMILES: *NC(=O)CC(S*)C(=O)O] |
| 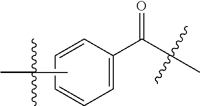 | 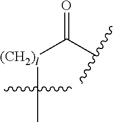 |
| 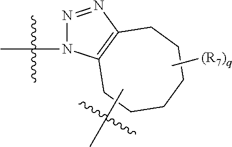 | 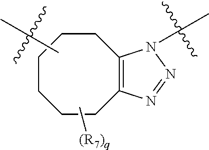 |
| 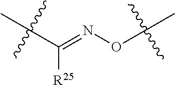 | 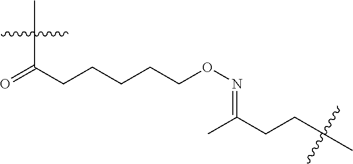 [SMILES: *CC/C(C)=N/OCCCCCC(*)=O] |
| 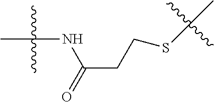 [SMILES: *NC(=O)CCS*] | 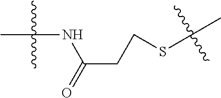 [SMILES: *NC(=O)CCS*] |
| 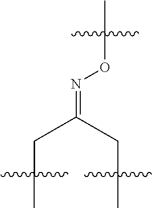 [SMILES: *CC(C*)=NO*] | 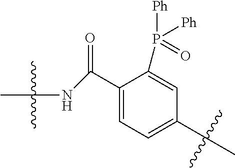 [SMILES: *NC(=O)c1ccc(*)cc1P(=O)(c1ccccc1)c1ccccc1] |
| 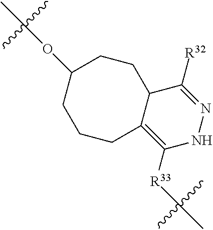 | 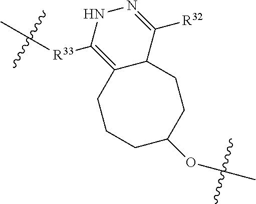 |
| 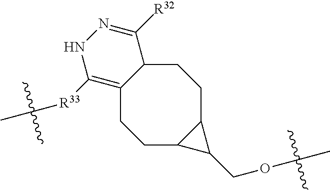 | 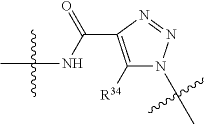 |
| 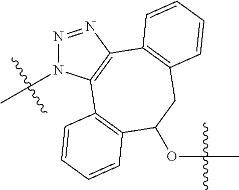 [SMILES: *OC1Cc2ccccc2-c2nnn(*)c2-c2ccccc21] | 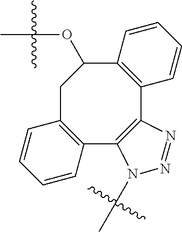 [SMILES: *OC1Cc2ccccc2-c2c(nnn2*)-c2ccccc21] |
| 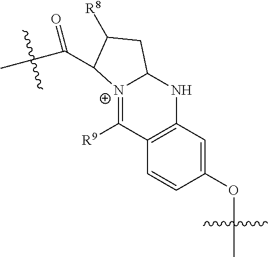 | 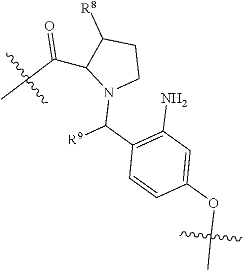 |
| 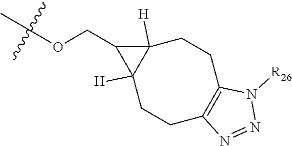 | 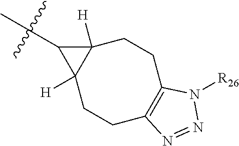 |
| | 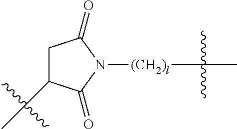 |
| | 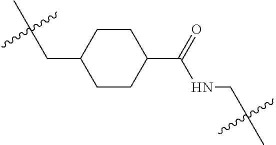 [SMILES: *CNC(=O)C1CCC(C*)CC1] |
| | 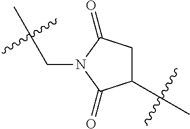 [SMILES: *CN1C(=O)CC(*)C1=O] |
| | 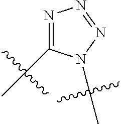 [SMILES: *c1nnnn1*] |
| | 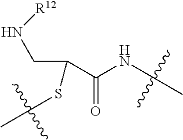 |
| | 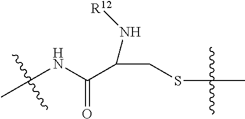 |
| | 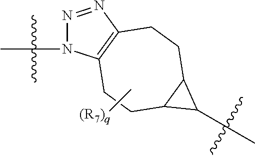 |
| | 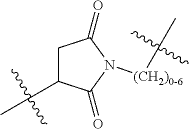 [SMILES: *CN1C(=O)CC(*)C1=O] |
| | 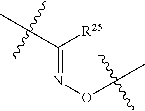 |
| | 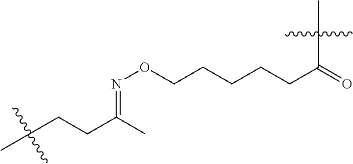 [SMILES: *CC/C(C)=N/OCCCCCC(*)=O] |
| | 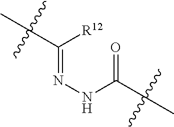 |
| | 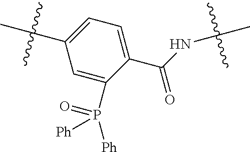 [SMILES: *NC(=O)c1ccc(*)cc1P(=O)(c1ccccc1)c1ccccc1] |
| | 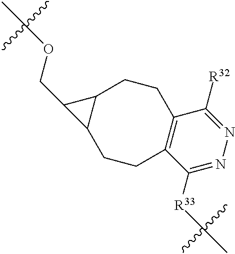 |
| | 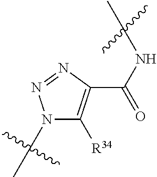 |
| | 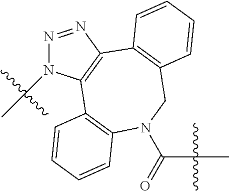 [SMILES: *C(=O)N1Cc2ccccc2-c2nnn(*)c2-c2ccccc21] |
| | 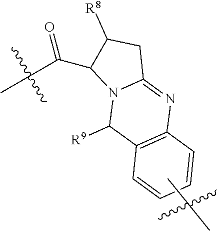 |
| | 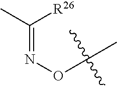 |

each R⁷ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
each R¹² is independently selected from H and C₁-C₆alkyl
R⁸ is H or methyl;
R⁹ is H, —CH₃ or phenyl;
each R²⁵ is independently selected from H or C₁₋₄ alkyl;
each R¹⁸ is independently selected from a C₁-C₆alkyl, a C₁-C₆alkyl which is substituted with azido and a C₁-C₆alkyl which is substituted with 1 to 5 hydroxyl;
q is 0, 1, 2 or 3;
I is 1, 2, 3, 4, 5 or 6;
R²⁶ is 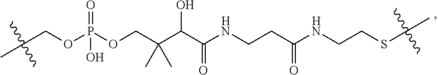 [SMILES: *COP(=O)(O)OCC(C)(C)C(O)C(=O)NCCC(=O)NCCS*] 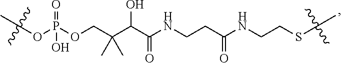 [SMILES: *OP(=O)(O)OCC(C)(C)C(O)C(=O)NCCC(=O)NCCS*] 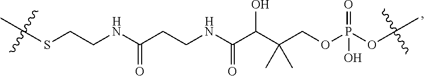 [SMILES: *OP(=O)(O)OCC(C)(C)C(O)C(=O)NCCC(=O)NCCS*] 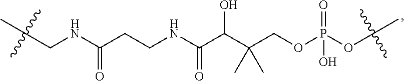 [SMILES: *CNC(=O)CCNC(=O)C(O)C(C)(C)COP(=O)(O)O*] 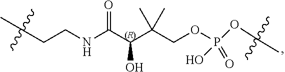 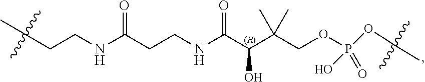 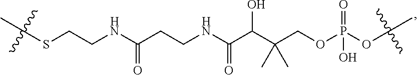 [SMILES: *OP(=O)(O)OCC(C)(C)C(O)C(=O)NCCC(=O)NCCS*] 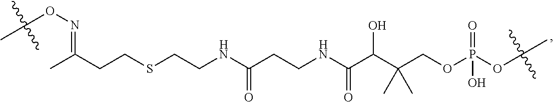 [SMILES: *O/N=C(\C)CCSCCNC(=O)CCNC(=O)C(O)C(C)(C)COP(=O)(O)O*] 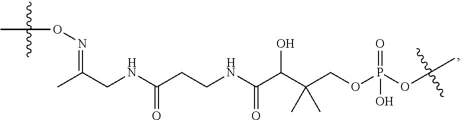 [SMILES: *O/N=C(\C)CNC(=O)CCNC(=O)C(O)C(C)(C)COP(=O)(O)O*] 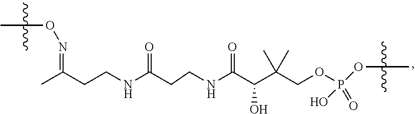 [SMILES: *O/N=C(\C)CCNC(=O)CCNC(=O)[C@@H](O)C(C)(C)COP(=O)(O)O*] 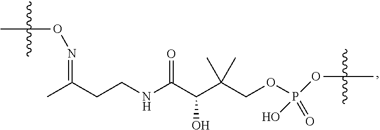 [SMILES: *O/N=C(\C)CCNC(=O)[C@@H](O)C(C)(C)COP(=O)(O)O*] 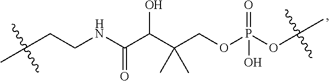 [SMILES: *CCNC(=O)C(O)C(C)(C)COP(=O)(O)O*] 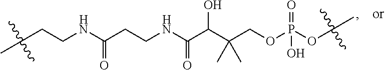 [SMILES: *CCNC(=O)CCNC(=O)C(O)C(C)(C)COP(=O)(O)O*]
or 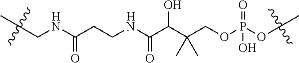 [SMILES: *CNC(=O)CCNC(=O)C(O)C(C)(C)COP(=O)(O)O*]
R³² is independently selected from H, C₁₋₄ alkyl, phenyl, pyrimidine and pyridine;
R³³ is independently selected from 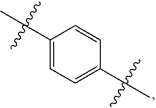 [SMILES: *c1ccc(*)cc1] 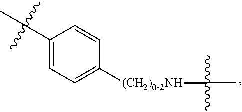 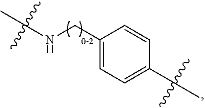 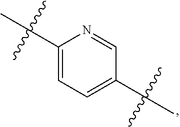 [SMILES: *c1ccc(*)nc1] 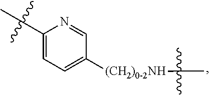 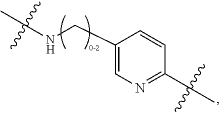 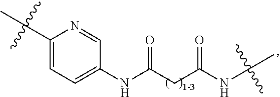
and 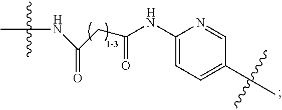
R³⁴ is independently selected from H, C₁₋₄ alkyl, C₁₋₆ haloalkyl, and
R^{aa} is an amino acid side chain.

As used herein, when a partial structure of a compound is illustrated, a wavy line ( [symbol] ) indicates the point of attachment of the partial structure to the rest of the molecule.

The terms “self-immolative spacer” and “self-immolative group”, as used herein, refer a moiety comprising one or more triggering groups (TG) which are activated by acid-induced cleavage, peptidase-induced cleavage, esterase-induced cleavage, glycosidase induced cleavage, phosphodiesterase induced cleavage, phosphatase induced cleavage, protease induced cleavage, lipase induced cleavage or disulfide bond cleavage, and after activation the protecting group is removed, which generates a cascade of disassembling reactions leading to the temporally sequential release of a leaving group. Such cascade of reactions can be, but not limited to, 1,4-, 1,6- or 1,8-elimination reactions.

Non-limiting examples of self-immolative spacer or group include:

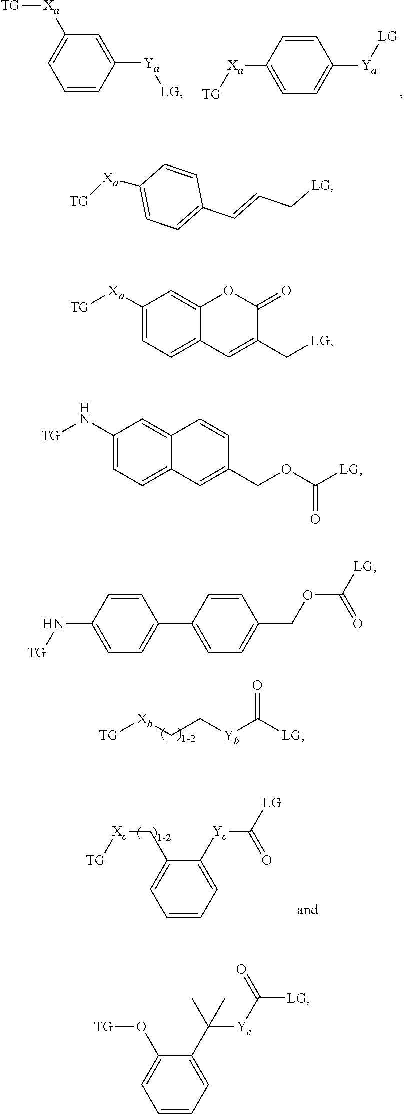

wherein such groups can be optionally substituted, and
wherein:
   TG is a triggering group;
   Xₐ is O, NH or S;
   X_{b} is O, NH, NCH₃ or S;
   X_{c} is O or NH;
   Yₐ is CH₂, CH₂O or CH₂NH;
   Y_{b} is CH₂, O or NH;
   Y_{c} is a bond, CH₂, O or NH, and
   LG is a leaving group such as a Drug moiety (D) of the Linker-Drug group of the invention.

Additional non-limiting examples of self-immolative spacers are described in Angew. Chem. Int. Ed. 2015, 54, 7492-7509.

In certain embodiment the self-immolative spacer is moiety having the structure

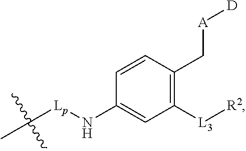

where Lp is an enzymatically cleavable bivalent peptide spacer and A, D, L₃ and R² are as defined herein.

In preferred embodiments, the self-immolative spacer is moiety having the structure

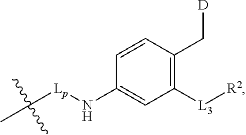

where Lp is an enzymatically cleavable bivalent peptide spacer and D, L₃ and R² are as defined herein. In some embodiments, D is a quaternized tertiary amine-containing panRAS inhibitor.

In other preferred embodiments, the self-immolative spacer is moiety having the structure

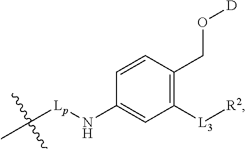

where Lp is an enzymatically cleavable bivalent peptide spacer and D, L₃ and R² are as defined herein.

The term “hydrophilic moiety”, as used herein, refers to moiety that is has hydrophilic properties which increases the aqueous solubility of the Drug moiety (D) when the Drug moiety (D) is attached to the conjugate linker group of the invention. Examples of such hydrophilic groups include, but are not limited to, polyethylene glycols, polyalkylene glycols, sugars, oligosaccharides, polypeptides a C₂-C₆alkyl substituted with 1 to 3

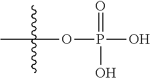 [SMILES: *OP(=O)(O)O]

groups.

### Drug Moieties

In some embodiments, an intermediate, which is the precursor of the conjugate linker moiety, is reacted with the drug moiety (e.g., the panRAS inhibitor) under appropriate conditions. In some embodiments, reactive groups are used on the drug and/or the intermediate or conjugate linker. The product of the reaction between the drug and the intermediate, or the derivatized drug (drug plus conjugate linker), is subsequently reacted with the antibody or antigen-binding fragment under conditions that facilitate conjugation of the drug and intermediate or derivatized drug and antibody or antigen-binding fragment. Alternatively, the intermediate or conjugate linker may first be reacted with the antibody or antigen-binding fragment, or a derivatized antibody or antigen-binding fragment, and then reacted with the drug or derivatized drug.

A number of different reactions are available for covalent attachment of the drug moiety and/or conjugate linker moiety to the antibody or antigen-binding fragment. This is often accomplished by reaction of one or more amino acid residues of the antibody or antigen-binding fragment, including the amine groups of lysine, the free carboxylic acid groups of glutamic acid and aspartic acid, the sulfhydryl groups of cysteine, and the various moieties of the aromatic amino acids. For instance, non-specific covalent attachment may be undertaken using a carbodiimide reaction to link a carboxy (or amino) group on a drug moiety to an amino (or carboxy) group on an antibody or antigen-binding fragment. Additionally, bifunctional agents such as dialdehydes or imidoesters may also be used to link the amino group on a drug moiety to an amino group on an antibody or antigen-binding fragment. Also available for attachment of drugs (e.g., a panRAS inhibitor) to binding agents is the Schiff base reaction. This method involves the periodate oxidation of a drug that contains glycol or hydroxy groups, thus forming an aldehyde which is then reacted with the binding agent. Attachment occurs via formation of a Schiff base with amino groups of the binding agent. Isothiocyanates may also be used as coupling agents for covalently attaching drugs to binding agents. Other techniques are known to the skilled artisan and within the scope of the present disclosure. Examples of drug moieties that can be generated and linked to an antibody or antigen-binding fragment using various chemistries known to in the art include panRAS inhibitors, e.g., the panRAS inhibitors described and exemplified herein.

Suitable drug moieties may comprise a compound of the formulas (Ia), (I), (Ic), (If), (Ig), (Ih), (Ij), (Ik), (Im), or (In) or an enantiomer, diastereoisomer, and/or addition salt thereof with a pharmaceutically acceptable acid or base. Additionally, the drug moiety may comprise any compounds of the panRAS inhibitor (D) described herein.

In some embodiments, the drug moiety (D) comprises a formula selected from Table A2.

In some embodiments, the drug moiety (D) comprises a panRAS inhibitor known in the art, for example, disclosed in WO2021/091956 or WO2022/060836, where are hereby incorporated by reference in their entirety.

In some embodiments, the drug moiety (D) comprises a panRAS inhibitor selected from:

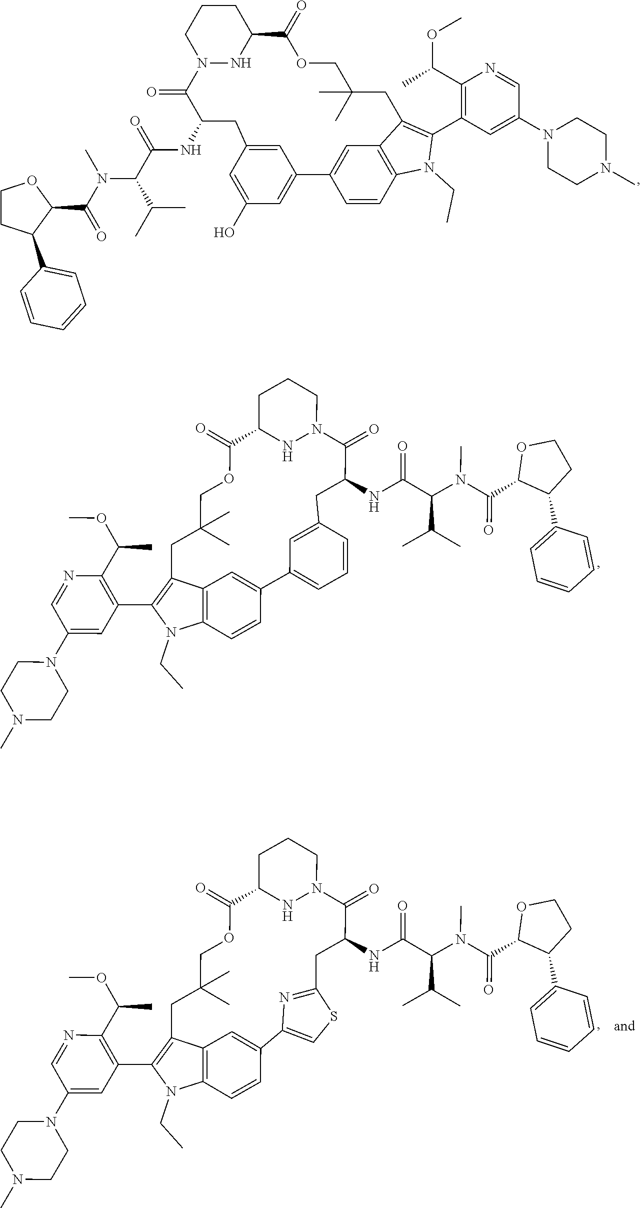

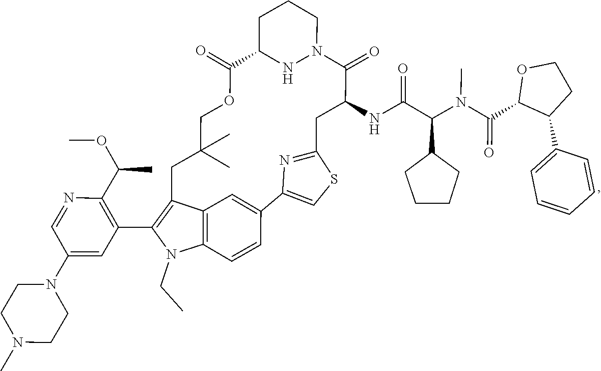 [SMILES: CCn1c(-c2cc(N3CCN(C)CC3)cnc2[C@H](C)OC)c2c3cc(ccc31)-c1csc(n1)C[C@H](NC(=O)[C@H](C1CCCC1)N(C)C(=O)[C@@H]1OCC[C@@H]1c1ccccc1)C(=O)N1CCC[C@H](N1)C(=O)OCC(C)(C)C2]

In some embodiments, the linker-drug (or “linker-payload”) moiety -(L-D) may comprise a compounds in Table B or an enantiomer, diastereoisomer, deuterated derivative, and/or a pharmaceutically acceptable salt of any of the foregoing.

### Definitions for Terms in Drug Moieties

Those skilled in the art will appreciate that certain compounds described herein can exist in one or more different isomeric (e.g., stereoisomers, geometric isomers, atropisomers, tautomers) or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

Compounds described herein can be asymmetric (e.g., having one or more stereocenters). All stereoisomers, such as enantiomers and diastereomers, are intended unless otherwise indicated.

Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion.

Unless otherwise stated, structures depicted herein are also meant to include compounds that differ only in the presence of one or more isotopically enriched atoms. Exemplary isotopes that can be incorporated into compounds of the present invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ³C- or ¹⁴C-enriched carbon. Positron emitting isotopes such as ¹⁵O, ¹³N, ¹¹C, and ¹³F are useful for positron emission tomography (PET) studies to examine substrate receptor occupancy.

Preparations of isotopically labelled compounds are known to those of skill in the art. For example, isotopically labeled compounds can generally be prepared by following procedures analogous to those disclosed for compounds of the present invention described herein, by substituting an isotopically labeled reagent for a non-isotopically labeled reagent.

As is known in the art, many chemical entities can adopt a variety of different solid forms such as, for example, amorphous forms or crystalline forms (e.g., polymorphs, hydrates, solvate). In some embodiments, compounds of the present invention may be utilized in any such form, including in any solid form. In some embodiments, compounds described or depicted herein may be provided or utilized in hydrate or solvate form.

At various places in the present specification, substituents of compounds of the present disclosure are disclosed in groups or in ranges. It is specifically intended that the present disclosure includes each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. Furthermore, where a compound includes a plurality of positions at which substituents are disclosed in groups or in ranges, unless otherwise indicated, the present disclosure is intended to cover individual compounds and groups of compounds (e.g., genera and subgenera) containing each and every individual subcombination of members at each position.

The term “optionally substituted X” (e.g., “optionally substituted alkyl”) is intended to be equivalent to “X, wherein X is optionally substituted” (e.g., “alkyl, wherein said alkyl is optionally substituted”). It is not intended to mean that the feature “X” (e.g. alkyl) per se is optional. As described herein, certain compounds of interest may contain one or more “optionally substituted” moieties. In general, the term “substituted”, whether preceded by the term “optionally” or not, means that one or more hydrogens of the designated moiety are replaced with a suitable substituent, e.g., any of the substituents or groups described herein. Unless otherwise indicated, an “optionally substituted” group may have a suitable substituent at each substitutable position of the group, and when more than one position in any given structure may be substituted with more than one substituent selected from a specified group, the substituent may be either the same or different at every position. For example, in the term “optionally substituted C₁-C₆ alkyl-C₂-C₉ heteroaryl,” the alkyl portion, the heteroaryl portion, or both, may be optionally substituted. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group may be, independently, deuterium; halogen; —(CH₂)₀₋₄R^{∘}; —(CH₂)₀₋₄OR^{∘}; —O(CH₂)₀₋₄R^{∘}; —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)0-1Ph which may be substituted with R^{∘}; —CH═CHPh, which may be substituted with R^{∘}; —(CH₂)₀₋₄O(CH₂)0-1-pyridyl which may be substituted with R^{∘}; 4-8 membered saturated or unsaturated heterocycloalkyl (e.g., pyridyl): 3-8 membered saturated or unsaturated cycloalkyl (e.g. cyclopropyl, cyclobutyl, or cyclopentyl); —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}2; —N(R^{∘})C(S)NR^{∘}2, —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂, —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄—C(O)—N(R^{∘})₂; —(CH₂)₀₋₄—C(O)—N(R^{∘})—S(O)₂—R^{∘}; —C(NCN)NR^{∘}2; —(CH₂)₀₋₄C(O)SR^{∘}; —(CH₂)₀₋₄C(O)OsiR^{∘}₃; —(CH₂)₀₋₄OC(O)R^{∘}; —OC(O)(CH₂)₀₋₄SR^{∘}; —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄ OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂: —(CH₂)₀₋₄S(O)R^{∘}; —N(R^{∘})S(O)₂NR^{∘}2; —N(R^{∘})S(O)₂R^{∘}; —N(OR^{∘})R^{∘}; —C(NOR^{∘})NR^{∘}₂, —C(NH)NR^{∘}₂, —P(O)₂R^{∘}; —P(O)R^{∘}₂; —P(O)(OR^{∘})₂; —OP(O)R^{∘}₂; —OP(O)(OR^{∘})₂; —OP(O)(OR^{∘})R^{∘}, —SiR^{∘}₃; —(C₁₋₄ straight or branched alkylene)O—N(R^{∘})₂; or —(C₁₋₄ straight or branched alkylene)C(O)O—N(R′)₂, wherein each R^{∘} may be substituted as defined below and is independently hydrogen, —C₁₋₆ aliphatic, —CH₂Ph, —O(CH₂)₀₋₁Ph, —CH₂-(5-6 membered heteroaryl ring), or a 3-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable divalent substituents on a saturated carbon atom of an “optionally substituted” group include the following: ═O, ═S, ═NNR*2, ═NNHC(O)R*, ═NNHC(O)OR*, ═NNHS(O)₂R*, ═NR*, ═NOR*, —O(C(R*2))₂-3O—, or —S(C(R*2))₂-3S-, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*2)₂-3O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R*, -(haloR*), —OH, —OR*, —O(haloR*), —CN, —C(O)OH, —C(O)OR*, —NH2, —NHR*, —NR*₂, or —NO₂, wherein each R* is unsubstituted or where preceded by “halo” is substituted only with one or more halogens, and is independently C₁₋₄ aliphatic, —CH₂Ph, —O(CH₂)0-1 Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}2, —C(O)R^{†}, —C(O)OR^{†}, —C(O)C(O)R^{†}, —C(O)CH₂C(O)R^{†}, —S(O)₂R^{†}, —S(O)₂NR^{†}2, —C(S)NR^{†}2, —C(NH)NR^{†}2, or —N(R^{†})S(O)₂R^{†}; wherein each R^{†} is independently hydrogen, C₁₋₆ aliphatic which may be substituted as defined below, unsubstituted -Oph, or an unsubstituted 3-6-membered saturated, partially unsaturated, or aryl ring having 0-4 heteroatoms independently selected from nitrogen, oxygen, or sulfur, or, notwithstanding the definition above, two independent occurrences of Rt, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on an aliphatic group of Rt are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•}is unsubstituted or where preceded by “halo” is substituted only with one or more halogens, and is independently C₁₋₄ aliphatic, —CH₂Ph, —O(CH₂)0-1 Ph, or a 5-6-membered saturated, partially unsaturated, or aryl ring having 0-4 heteroatoms independently selected from nitrogen, oxygen, or sulfur. Suitable divalent substituents on a saturated carbon atom of Rt include ═O and ═S.

The term “acetyl,” as used herein, refers to the group —C(O)CH₃.

The term “alkoxy,” as used herein, refers to a —O—C₁-C₂₀ alkyl group, wherein the alkoxy group is attached to the remainder of the compound through an oxygen atom.

The term “alkyl,” as used herein, refers to a saturated, straight or branched monovalent hydrocarbon group containing from 1 to 20 (e.g., from 1 to 10 or from 1 to 6) carbons. In some embodiments, an alkyl group is unbranched (i.e., is linear); in some embodiments, an alkyl group is branched. Alkyl groups are exemplified by, but not limited to, methyl, ethyl, n- and iso-propyl, n-, sec-, iso- and tert-butyl, and neopentyl.

The term “heteroalkyl,” as used herein, refers to an “alkyl” group, as defined herein, in which at least one carbon atom has been replaced with a heteroatom (e.g., an O, N, or S atom). The heteroatom may appear in the middle or at the end of the radical.

The term “alkylene,” as used herein, represents a saturated divalent hydrocarbon group derived from a straight or branched chain saturated hydrocarbon by the removal of two hydrogen atoms, and is exemplified by methylene, ethylene, isopropylene, and the like. The term “Cx-Cy alkylene” represents alkylene groups having between x and y carbons. Exemplary values for x are 1, 2, 3, 4, 5, and 6, and exemplary values for y are 2, 3, 4, 5, , 7, 8, 9, 10, 12, 14, 16, 18, or 20 (e.g., C₁-C₆, C₁-C₁₀, C₂-C₂₀, C₂-C₂₀, C₂-C₁₀, or C₂-C₂₀ alkylene). In some embodiments, the alkylene can be further substituted with 1, 2, 3, or 4 substituent groups as defined herein.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, and 2-butenyl. Alkenyls include both cis and trans isomers. The term “alkenylene,” as used herein, represents a divalent straight or branched chain groups of, unless otherwise specified, from 2 to 20 carbons (e.g., from 2 to 6 or from 2 to 10 carbons) containing one or more carbon-carbon double bonds.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, and 1-propynyl.

The term “amino,” as used herein, represents —N(R^{†})₂, e.g., —NH₂ and —N(CH₃)₂.

The term “aminoalkyl,” as used herein, represents an alkyl moiety substituted on one or more carbon atoms with one or more amino moieties.

The term “amino acid,” as described herein, refers to a molecule having a side chain, an amino group, and an acid group (e.g., —CO₂H or —SO₃H), wherein the amino acid is attached to the parent molecular group by the side chain, amino group, or acid group (e.g., the side chain). As used herein, the term “amino acid” in its broadest sense, refers to any compound or substance that can be incorporated into a polypeptide chain, e.g., through formation of one or more peptide bonds. In some embodiments, an amino acid has the general structure H₂N—C(H)(R^{A*})—COOH, wherein R^{A*} is any chemically feasible substituent described herein. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. Exemplary amino acids include alanine, arginine, asparagine, aspartic acid, cysteine, glutamic acid, glutamine, glycine, histidine, optionally substituted hydroxylnorvaline, isoleucine, leucine, lysine, methionine, norvaline, ornithine, phenylalanine, proline, pyrrolysine, selenocysteine, serine, taurine, threonine, tryptophan, tyrosine, and valine.

The term “aryl,” as used herein, represents a monovalent monocyclic, bicyclic, or multicyclic ring system formed by carbon atoms, wherein the ring attached to the pendant group is aromatic. Examples of aryl groups are phenyl, naphthyl, phenanthrenyl, and anthracenyl. An aryl ring can be attached to its pendant group at any heteroatom or carbon ring atom that results in a stable structure and any of the ring atoms can be optionally substituted unless otherwise specified. In some embodiments, the aryl refers to a phenyl, naphthyl, biphenyl or indenyl group.

The term “C₀,” as used herein, represents a bond. For example, part of the term —N(C(O)—(C₀-C₅ alkylene-H)— includes —N(C(O)—(C₀ alkylene-H)—, which is also represented by —N(C(O)—H)—.

The terms “carbocyclic” and “carbocyclyl,” as used herein, refer to a monovalent, optionally substituted C₃-C₁₂ monocyclic, bicyclic, or tricyclic ring structure, which may be bridged, fused or spirocyclic, in which all the rings are formed by carbon atoms and at least one ring is non-aromatic.

Carbocyclic structures include cycloalkyl, cycloalkenyl, and cycloalkynyl groups. Examples of carbocyclyl groups are cyclohexyl, cyclohexenyl, cyclooctynyl, 1,2-dihydronaphthyl, 1,2,3,4-tetrahydronaphthyl, fluorenyl, indenyl, indanyl, decalinyl, and the like. A carbocyclic ring can be attached to its pendant group at any ring atom that results in a stable structure and any of the ring atoms can be optionally substituted unless otherwise specified.

The term “carbonyl,” as used herein, represents a C(O) group, which can also be represented as C═O.

The term “carboxyl,” as used herein, means —CO₂H, (C═O)(OH), COOH, or C(O)OH or the unprotonated counterparts.

The term “cyano,” as used herein, represents a —CN group.

The term “diastereomer,” as used herein, means stereoisomers that are not mirror images of one another and are non-superimposable on one another.

The term “enantiomer,” as used herein, means each individual optically active form of a compound of the invention, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “haloalkyl,” as used herein, represents an alkyl moiety substituted on one or more carbon atoms with one or more of the same of different halogen moieties.

The term “halogen,” as used herein, represents a halogen selected from bromine, chlorine, iodine, or fluorine.

The term “heteroalkyl,” as used herein, refers to an “alkyl” group, as defined herein, in which at least one carbon atom has been replaced with a heteroatom (e.g., an O, N, or S atom). The heteroatom may appear in the middle or at the end of the radical.

The term “heteroaryl,” as used herein, represents a monovalent, monocyclic or polycyclic ring structure that contains at least one fully aromatic ring: i.e., they contain 4n+2 pi electrons within the monocyclic or polycyclic ring system and contains at least one ring heteroatom selected from N, O, or S in that aromatic ring. Exemplary unsubstituted heteroaryl groups are of 1 to 12 (e.g., 1 to 11, 1 to 10, 1 to 9, 2 to 12, 2 to 11, 2 to 10, or 2 to 9) carbons. The term “heteroaryl” includes bicyclic, tricyclic, and tetracyclic groups in which any of the above heteroaromatic rings is fused to one or more, aryl or carbocyclic rings, e.g., a phenyl ring, or a cyclohexane ring. Examples of heteroaryl groups include, but are not limited to, pyridyl, pyrazolyl, benzooxazolyl, benzoimidazolyl, benzothiazolyl, imidazolyl, thiazolyl, quinolinyl, tetrahydroquinolinyl, and 4-azaindolyl. A heteroaryl ring can be attached to its pendant group at any ring atom that results in a stable structure and any of the ring atoms can be optionally substituted unless otherwise specified. In some embodiments, the heteroaryl is substituted with 1, 2, 3, or 4 substituents groups. In some embodiments, the heteroaryl any mono- or bi-cyclic group composed of from 5 to 10 ring members, having at least one aromatic moiety and containing from 1 to 4 hetero atoms selected from oxygen, sulfur and nitrogen (including quaternary nitrogens).

The term “heterocycloalkyl,” as used herein, represents a monovalent monocyclic, bicyclic or polycyclic ring system, which may be bridged, fused or spirocyclic, wherein at least one ring is non-aromatic and wherein the non-aromatic ring contains one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocycloalkyl groups are of 1 to 12 (e.g., 1 to 11, 1 to 10, 1 to 9, 2 to 12, 2 to 11, 2 to 10, or 2 to 9) carbons.

The term “heterocycloalkyl” also represents a heterocyclic compound having a bridged multicyclic structure in which one or more carbons or heteroatoms bridges two non-adjacent members of a monocyclic ring, e.g., a quinuclidinyl group. The term “heterocycloalkyl” includes bicyclic, tricyclic, and tetracyclic groups in which any of the above heterocyclic rings is fused to one or more aromatic, carbocyclic, heteroaromatic, or heterocyclic rings, e.g., an aryl ring, a cyclohexane ring, a cyclohexene ring, a cyclopentane ring, a cyclopentene ring, a pyridine ring, or a pyrrolidine ring.

Examples of heterocycloalkyl groups are pyrrolidinyl, piperidinyl, 1,2,3,4-tetrahydroquinolinyl, decahydroquinolinyl, dihydropyrrolopyridine, and decahydronapthyridinyl. A heterocycloalkyl ring can be attached to its pendant group at any ring atom that results in a stable structure and any of the ring atoms can be optionally substituted unless otherwise specified.

The term “hydroxy,” as used herein, represents a —OH group.

The term “hydroxyalkyl,” as used herein, represents an alkyl moiety substituted on one or more carbon atoms with one or more —OH moieties.

The term “isomer,” as used herein, means any tautomer, stereoisomer, atropiosmer, enantiomer, or diastereomer of any compound of the invention. It is recognized that the compounds of the invention can have one or more chiral centers or double bonds and, therefore, exist as stereoisomers, such as double-bond isomers (i.e., geometric E/Z isomers) or diastereomers (e.g., enantiomers (i.e., (+) or (−)) or cis/trans isomers). According to the invention, the chemical structures depicted herein, and therefore the compounds of the invention, encompass all the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the invention can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

As used herein, the term “drug linker” refers to a divalent organic moiety connecting moiety B^{D} to moiety W^{D} in a compound of Formula I, such that the resulting compound is capable of achieving an IC50 of 2 μM or less in the Ras-RAF disruption assay protocol provided in the Examples below, and provided here:
The purpose of this biochemical assay is to measure the ability of test compounds to facilitate ternary complex formation between a nucleotide-loaded Ras isoform and cyclophilin A; the resulting ternary complex disrupts binding to a BRAF^{R8D} construct, inhibiting Ras signaling through a RAF effector.
In assay buffer containing 25 mM HEPES pH 7.3, 0.002% Tween20, 0.1% BSA, 100 mM NaCl and 5 mM MgCl₂, tagless Cyclophilin A, His6-K-Ras-GMPPNP (or other Ras variant), and GST-BRAF^{RBD} are combined in a 384-well assay plate at final concentrations of 25 μM, 12.5 nM and 50 nM, respectively. Compound is present in plate wells as a 10-point 3-fold dilution series starting at a final concentration of 30 μM. After incubation at 25° C. for 3 hours, a mixture of Anti-His Eu—W1024 and anti-GST allophycocyanin is then added to assay sample wells at final concentrations of 10 nM and 50 nM, respectively, and the reaction incubated for an additional 1.5 hours. TR-FRET signal is read on a microplate reader (Ex 320 nm, Em 665/615 nm). Compounds that facilitate disruption of a Ras:RAF complex are identified as those eliciting a decrease in the TR-FRET ratio relative to DMSO control wells.

In some embodiments, the drug linker comprises 20 or fewer linear atoms. In some embodiments, the drug linker comprises 15 or fewer linear atoms. In some embodiments, the drug linker comprises 10 or fewer linear atoms. In some embodiments, the drug linker has a molecular weight of under 500 g/mol. In some embodiments, the drug linker has a molecular weight of under 400 g/mol. In some embodiments, the drug linker has a molecular weight of under 300 g/mol. In some embodiments, the drug linker has a molecular weight of under 200 g/mol. In some embodiments, the drug linker has a molecular weight of under 100 g/mol. In some embodiments, the drug linker has a molecular weight of under 50 g/mol.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers or conformers of the basic molecular structure, including atropisomers. Some compounds of the present invention may exist in different tautomeric forms, all of the latter being included within the scope of the present invention.

The term “sulfonyl” or “sulphonyl,” as used herein, represents an —S(O)₂— group.

The term “thiocarbonyl,” as used herein, refers to a —C(S)— group.

### Drug Loading

Drug loading is represented by p, and is also referred to herein as the drug-to-antibody ratio (DAR). Drug loading may range from 1 to 16 drug moieties per antibody or antigen-binding fragment. In some embodiments, p is an integer from 1 to 16. In some embodiments, p is an integer from 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 to 2. In some embodiments, p is an integer from 2 to 10, 2 to 9, 2 to 8, 2 to 7, 2 to 6, 2 to 5, 2 to 4, or 2 to 3. In some embodiments, p is an integer from 1 to 16. In some embodiments, p is an integer from 1 to 8. In some embodiments, p is an integer from 1 to 5. In some embodiments, p is an integer from 2 to 4. In some embodiments, p is 1, 2, 3, 4, 5, 6, 7, or 8. In some embodiments, p is 1. In some embodiments, p is 2. In some embodiments, p is 4.

Drug loading may be limited by the number of attachment sites on the antibody or antigen-binding fragment. In some embodiments, the conjugate linker moiety (L) of the ADC attaches to the antibody or antigen-binding fragment through a chemically active group on one or more amino acid residues on the antibody or antigen-binding fragment. For example, the conjugate linker may be attached to the antibody or antigen-binding fragment via a free amino, imino, hydroxyl, thiol, or carboxyl group (e.g., to the N- or C-terminus, to the epsilon amino group of one or more lysine residues, to the free carboxylic acid group of one or more glutamic acid or aspartic acid residues, or to the sulfhydryl group of one or more cysteine residues). The site to which the conjugate linker is attached can be a natural residue in the amino acid sequence of the antibody or antigen-binding fragment, or it can be introduced into the antibody or antigen-binding fragment, e.g., by DNA recombinant technology (e.g., by introducing a cysteine residue into the amino acid sequence) or by protein biochemistry (e.g., by reduction, pH adjustment, or hydrolysis).

In some embodiments, the number of drug moieties that can be conjugated to an antibody or antigen-binding fragment is limited by the number of free cysteine residues. For example, where the attachment is a cysteine thiol group, an antibody may have only one or a few cysteine thiol groups, or may have only one or a few sufficiently reactive thiol groups through which a conjugate linker may be attached. Generally, antibodies do not contain many free and reactive cysteine thiol groups that may be linked to a drug moiety. Indeed, most cysteine thiol residues in antibodies are involved in either interchain or intrachain disulfide bonds. Conjugation to cysteines can therefore, in some embodiments, require at least partial reduction of the antibody. Over-attachment of conjugate linker-toxin to an antibody may destabilize the antibody by reducing the cysteine residues available to form disulfide bonds. Therefore, an optimal drug:antibody ratio should increase potency of the ADC (by increasing the number of attached drug moieties per antibody) without destabilizing the antibody or antigen-binding fragment. In some embodiments, an optimal ratio may be 2, 4, 6, or 8. In some embodiments, an optimal ratio may be 2 or 4.

In some embodiments, an antibody or antigen-binding fragment is exposed to reducing conditions prior to conjugation in order to generate one or more free cysteine residues. An antibody, in some embodiments, may be reduced with a reducing agent such as dithiothreitol (DTT) or tris(2-carboxyethyl)phosphine (TCEP), under partial or total reducing conditions, to generate reactive cysteine thiol groups. Unpaired cysteines may be generated through partial reduction with limited molar equivalents of TCEP, which can reduce the interchain disulfide bonds which link the light chain and heavy chain (one pair per H-L pairing) and the two heavy chains in the hinge region (two pairs per H—H pairing in the case of human IgG1) while leaving the intrachain disulfide bonds intact (Stefano et al. (2013) Methods Mol Biol. 1045:145-71). In embodiments, disulfide bonds within the antibodies are reduced electrochemically, e.g., by employing a working electrode that applies an alternating reducing and oxidizing voltage. This approach can allow for on-line coupling of disulfide bond reduction to an analytical device (e.g., an electrochemical detection device, an NMR spectrometer, or a mass spectrometer) or a chemical separation device (e.g., a liquid chromatograph (e.g., an HPLC) or an electrophoresis device (see, e.g., US 2014/0069822)). In some embodiments, an antibody is subjected to denaturing conditions to reveal reactive nucleophilic groups on amino acid residues, such as cysteine.

The drug loading of an ADC may be controlled in different ways, e.g., by: (i) limiting the molar excess of drug-linker intermediate or conjugate linker reagent relative to antibody; (ii) limiting the conjugation reaction time or temperature; (iii) partial or limiting reductive conditions for cysteine thiol modification; and/or (iv) engineering by recombinant techniques the amino acid sequence of the antibody such that the number and position of cysteine residues is modified for control of the number and/or position of linker-drug attachments.

In some embodiments, free cysteine residues are introduced into the amino acid sequence of the antibody or antigen-binding fragment. For example, cysteine engineered antibodies can be prepared wherein one or more amino acids of a parent antibody are replaced with a cysteine amino acid. Any form of antibody may be so engineered, i.e. mutated. For example, a parent Fab antibody fragment may be engineered to form a cysteine engineered Fab referred to as a “ThioFab.” Similarly, a parent monoclonal antibody may be engineered to form a “ThioMab.” A single site mutation yields a single engineered cysteine residue in a ThioFab, whereas a single site mutation yields two engineered cysteine residues in a ThioMab, due to the dimeric nature of the IgG antibody. DNA encoding an amino acid sequence variant of the parent polypeptide can be prepared by a variety of methods known in the art (see, e.g., the methods described in WO 2006/034488). These methods include, but are not limited to, preparation by site-directed (or oligonucleotide-mediated) mutagenesis, PCR mutagenesis, and cassette mutagenesis of an earlier prepared DNA encoding the polypeptide. Variants of recombinant antibodies may also be constructed by restriction fragment manipulation or by overlap extension PCR with synthetic oligonucleotides. ADCs of Formula (1) include, but are not limited to, antibodies that have 1, 2, 3, or 4 engineered cysteine amino acids (Lyon et al. (2012) Methods Enzymol. 502:123-38). In some embodiments, one or more free cysteine residues are already present in an antibody or antigen-binding fragment, without the use of engineering, in which case the existing free cysteine residues may be used to conjugate the antibody or antigen-binding fragment to a drug moiety.

Where more than one nucleophilic group reacts with a drug-linker intermediate or a conjugate linker moiety reagent followed by drug moiety reagent, in a reaction mixture comprising multiple copies of the antibody or antigen-binding fragment and conjugate linker moiety, then the resulting product can be a mixture of ADC compounds with a distribution of one or more drug moieties attached to each copy of the antibody or antigen-binding fragment in the mixture. In some embodiments, the drug loading in a mixture of ADCs resulting from a conjugation reaction ranges from 1 to 16 drug moieties attached per antibody or antigen-binding fragment. The average number of drug moieties per antibody or antigen-binding fragment (i.e., the average drug loading, or average p) may be calculated by any conventional method known in the art, e.g., by mass spectrometry (e.g., liquid chromatography-mass spectrometry (LC-MS)) and/or high-performance liquid chromatography (e.g., HIC-HPLC). In some embodiments, the average number of drug moieties per antibody or antigen-binding fragment is determined by liquid chromatography-mass spectrometry (LC-MS). In some embodiments, the average number of drug moieties per antibody or antigen-binding fragment is from about 1.5 to about 3.5, about 2.5 to about 4.5, about 3.5 to about 5.5, about 4.5 to about 6.5, about 5.5 to about 7.5, about 6.5 to about 8.5, or about 7.5 to about 9.5. In some embodiments, the average number of drug moieties per antibody or antigen-binding fragment is from about 2 to about 4, about 3 to about 5, about 4 to about 6, about 5 to about 7, about 6 to about 8, about 7 to about 9, about 2 to about 8, or about 4 to about 8.

In some embodiments, the average number of drug moieties per antibody or antigen-binding fragment is about 2. In some embodiments, the average number of drug moieties per antibody or antigen-binding fragment is about 1.5, about 1.6, about 1.7, about 1.8, about 1.9, about 2, about 2.1, about 2.2, about 2.3, about 2.4, or about 2.5. In some embodiments, the average number of drug moieties per antibody or antigen-binding fragment is 2.

In some embodiments, the average number of drug moieties per antibody or antigen-binding fragment is about 4. In some embodiments, the average number of drug moieties per antibody or antigen-binding fragment is about 3.5, about 3.6, about 3.7, about 3.8, about 3.9, about 4, about 4.1, about 4.2, about 4.3, about 4.4, or about 4.5. In some embodiments, the average number of drug moieties per antibody or antigen-binding fragment is 4.

In some embodiments, the term “about,” as used with respect to the average number of drug moieties per antibody or antigen-binding fragment, means plus or minus 20%, 15%, 10%, 5%, or 1%. In one embodiment, the term “about” refers to a range of values which are 10% more or less than the specified value. In another embodiment, the term “about” refers to a range of values which are 5% more or less than the specified value. In another embodiment, the term “about” refers to a range of values which are 1% more or less than the specified value.

Individual ADC compounds, or “species,” may be identified in the mixture by mass spectroscopy and separated by, e.g., UPLC or HPLC, e.g. hydrophobic interaction chromatography (HIC-HPLC). In some embodiments, a homogeneous or nearly homogenous ADC product with a single loading value may be isolated from the conjugation mixture, e.g., by electrophoresis or chromatography.

In some embodiments, higher drug loading (e.g., p>16) may cause aggregation, insolubility, toxicity, or loss of cellular permeability of certain antibody-drug conjugates. Higher drug loading may also negatively affect the pharmacokinetics (e.g., clearance) of certain ADCs. In some embodiments, lower drug loading (e.g., p<2) may reduce the potency of certain ADCs against target-expressing cells. In some embodiments, the drug loading for an ADC of the present disclosure ranges from about 2 to about 16, about 2 to about 10, about 2 to about 8; from about 2 to about 6; from about 2 to about 5; from about 3 to about 5; from about 2 to about 4; or from about 4 to about 8.

In some embodiments, a drug loading and/or an average drug loading of about 2 is achieved, e.g., using partial reduction of intrachain disulfides on the antibody or antigen-binding fragment, and provides beneficial properties. In some embodiments, a drug loading and/or an average drug loading of about 4 or about 6 or about 8 is achieved, e.g., using partial reduction of intrachain disulfides on the antibody or antigen-binding fragment, and provides beneficial properties. In some embodiments, a drug loading and/or an average drug loading of less than about 2 may result in an unacceptably high level of unconjugated antibody species, which can compete with the ADC for binding to a target antigen and/or provide for reduced treatment efficacy. In some embodiments, a drug loading and/or average drug loading of more than about 16 may result in an unacceptably high level of product heterogeneity and/or ADC aggregation. A drug loading and/or an average drug loading of more than about 16 may also affect stability of the ADC, due to loss of one or more chemical bonds required to stabilize the antibody or antigen-binding fragment.

The present disclosure includes methods of producing the described ADCs. Briefly, the ADCs comprise an antibody or antigen-binding fragment (e.g., anti-EphA2 or anti-B7-H3 antibody or antigen-binding fragment), a drug moiety (e.g., a panRAS inhibitor), and a conjugate linker that joins the drug moiety and the antibody or antigen-binding fragment. In some embodiments, the ADCs can be prepared using a conjugate linker having reactive functionalities for covalently attaching to the drug moiety and to the antibody or antigen-binding fragment. In some embodiments, the antibody or antigen-binding fragment is functionalized to prepare a functional group that is reactive with a conjugate linker or a drug-linker intermediate. For example, in some embodiments, a cysteine thiol of an antibody or antigen-binding fragment can form a bond with a reactive functional group of a conjugate linker or a drug-linker intermediate to make an ADC. In some embodiments, an antibody or antigen-binding fragment is prepared with bacterial transglutaminase (BTG)-reactive glutamines specifically functionalized with an amine containing cyclooctyne BCN (N-[(1R,8S,9s)-Bicyclo[6.1.0]non-4-yn-9-ylmethyloxycarbonyl]-1,8-diamino-3,6-dioxaoctane) moiety. In some embodiments, site-specific conjugation of a conjugate linker or a drug-linker intermediate to a BCN moiety of an antibody or antigen-binding fragment is performed, e.g., as described and exemplified herein. The generation of the ADCs can be accomplished by techniques known to the skilled artisan.

In some embodiments, an ADC is produced by contacting an antibody or antigen-binding fragment (e.g., anti-EphA2 or anti-B7-H3 antibody or antigen-binding fragment) with a conjugate linker and a drug moiety (e.g., a panRAS inhibitor) in a sequential manner, such that the antibody or antigen-binding fragment is covalently linked to the conjugate linker first, and then the pre-formed antibody-linker intermediate reacts with the drug moiety. The antibody-linker intermediate may or may not be subjected to a purification step prior to contacting the drug moiety. In other embodiments, an ADC is produced by contacting an antibody or antigen-binding fragment with a linker-drug compound pre-formed by reacting a conjugate linker with a drug moiety. The pre-formed linker-drug compound may or may not be subjected to a purification step prior to contacting the antibody or antigen-binding fragment. In other embodiments, the antibody or antigen-binding fragment contacts the conjugate linker and the drug moiety in one reaction mixture, allowing simultaneous formation of the covalent bonds between the antibody or antigen-binding fragment and the conjugate linker, and between the conjugate linker and the drug moiety. This method of producing ADCs may include a reaction, wherein the antibody or antigen-binding fragment contacts the antibody or antigen-binding fragment prior to the addition of the conjugate linker to the reaction mixture, and vice versa. In some embodiments, an ADC is produced by reacting an antibody or antigen-binding fragment with a conjugate linker joined to a drug moiety, such as a panRAS inhibitor, under conditions that allow conjugation.

The ADCs prepared according to the methods described above may be subjected to a purification step. The purification step may involve any biochemical methods known in the art for purifying proteins, or any combination of methods thereof. These include, but are not limited to, tangential flow filtration (TFF), affinity chromatography, ion exchange chromatography, any charge or isoelectric point-based chromatography, mixed mode chromatography, e.g., CHT (ceramic hydroxyapatite), hydrophobic interaction chromatography, size exclusion chromatography, dialysis, filtration, selective precipitation, or any combination thereof.

### Therapeutic Uses and Compositions

Disclosed herein are methods of using the compositions described herein, e.g., the disclosed ADC compounds and compositions, in treating a subject for a disorder, e.g., a cancer. Compositions, e.g., ADCs, may be administered alone or in combination with at least one additional inactive and/or active agent, e.g., at least one additional therapeutic agent, and may be administered in any pharmaceutically acceptable formulation, dosage, and dosing regimen. Treatment efficacy may be evaluated for toxicity as well as indicators of efficacy and adjusted accordingly. Efficacy measures include, but are not limited to, a cytostatic and/or cytotoxic effect observed in vitro or in vivo, reduced tumor volume, tumor growth inhibition, and/or prolonged survival.

Methods of determining whether an ADC exerts a cytostatic and/or cytotoxic effect on a cell are known. For example, the cytotoxic or cytostatic activity of an ADC can be measured by, e.g., exposing mammalian cells expressing a target antigen of the ADC in a cell culture medium; culturing the cells for a period from about 6 hours to about 6 days; and measuring cell viability (e.g., using a CellTiter-Glo® (CTG) or MTT cell viability assay). Cell-based in vitro assays may also be used to measure viability (proliferation), cytotoxicity, and induction of apoptosis (caspase activation) of the ADC.

For determining cytotoxicity, necrosis or apoptosis (programmed cell death) may be measured. Necrosis is typically accompanied by increased permeability of the plasma membrane, swelling of the cell, and rupture of the plasma membrane. Apoptosis can be quantitated, for example, by measuring DNA fragmentation. Commercial photometric methods for the quantitative in vitro determination of DNA fragmentation are available. Examples of such assays, including TUNEL (which detects incorporation of labeled nucleotides in fragmented DNA) and ELISA-based assays, are described in Biochemica (1999) 2:34-7 (Roche Molecular Biochemicals).

Apoptosis may also be determined by measuring morphological changes in a cell. For example, as with necrosis, loss of plasma membrane integrity can be determined by measuring uptake of certain dyes (e.g., a fluorescent dye such as, for example, acridine orange or ethidium bromide). A method for measuring apoptotic cell number has been described by Duke and Cohen, Current Protocols in Immunology (Coligan et al., eds. (1992) pp. 3.17.1-3.17.16). Cells also can be labeled with a DNA dye (e.g., acridine orange, ethidium bromide, or propidium iodide) and the cells observed for chromatin condensation and margination along the inner nuclear membrane. Apoptosis may also be determined, in some embodiments, by screening for caspase activity. In some embodiments, a Caspase-Glo® Assay can be used to measure activity of caspase-3 and caspase-7. In some embodiments, the assay provides a luminogenic caspase-3/7 substrate in a reagent optimized for caspase activity, luciferase activity, and cell lysis. In some embodiments, adding Caspase-Glo® 3/7 Reagent in an “add-mix-measure” format may result in cell lysis, followed by caspase cleavage of the substrate and generation of a “glow-type” luminescent signal, produced by luciferase. In some embodiments, luminescence may be proportional to the amount of caspase activity present, and can serve as an indicator of apoptosis. Other morphological changes that can be measured to determine apoptosis include, e.g., cytoplasmic condensation, increased membrane blebbing, and cellular shrinkage. Determination of any of these effects on cancer cells indicates that an ADC is useful in the treatment of cancers.

Cell viability may be measured, e.g., by determining in a cell the uptake of a dye such as neutral red, trypan blue, Crystal Violet, or ALAMAR™ blue (see, e.g., Page et al. (1993) Intl J Oncology 3:473-6). In such an assay, the cells are incubated in media containing the dye, the cells are washed, and the remaining dye, reflecting cellular uptake of the dye, is measured spectrophotometrically.

Cell viability may also be measured, e.g., by quantifying ATP, an indicator of metabolically active cells. In some embodiments, in vitro potency and/or cell viability of prepared ADCs or panRAS inhibitor compounds may be assessed using a CellTiter-Glo® (CTG) cell viability assay, as described in the examples provided herein. In this assay, in some embodiments, the single reagent (CellTiter-Glo® Reagent) is added directly to cells cultured in serum-supplemented medium. The addition of reagent results in cell lysis and generation of a luminescent signal proportional to the amount of ATP present. The amount of ATP is directly proportional to the number of cells present in culture.

Cell viability may also be measured, e.g., by measuring the reduction of tetrazolium salts. In some embodiments, in vitro potency and/or cell viability of prepared ADCs or panRAS inhibitor compounds may be assessed using an MTT cell viability assay, as described in the examples provided herein. In this assay, in some embodiments, the yellow tetrazolium MTT (3-(4, 5-dimethylthiazolyl-2)-2,5-diphenyltetrazolium bromide) is reduced by metabolically active cells, in part by the action of dehydrogenase enzymes, to generate reducing equivalents such as NADH and NADPH. The resulting intracellular purple formazan can then be solubilized and quantified by spectrophotometric means.

In certain aspects, the present disclosure features a method of killing, inhibiting or modulating the growth of a cancer cell or tissue by disrupting the expression and/or activity of panRAS (e.g., K-Ras (including splice variants KRAS4A and KRAS4B), H-Ras and N-Ras) and/or one or more upstream modulators or downstream targets thereof. The method may be used with any subject where disruption of panRAS (e.g., K-Ras (including splice variants KRAS4A and KRAS4B), H-Ras and N-Ras) expression and/or activity provides a therapeutic benefit. Subjects that may benefit from disrupting panRAS (e.g., K-Ras (including splice variants KRAS4A and KRAS4B), H-Ras and N-Ras) expression and/or activity include, but are not limited to, those having or at risk of having a cancer such as a tumor or a hematological cancer. In some embodiments, the cancer is a breast cancer including ER positive breast cancer, multiple myeloma, plasma cell myeloma, leukemia, lymphoma, sarcoma, gastric or stomach cancer, acute myeloid leukemia, bladder cancer, brain cancer, bone marrow cancer, cervical cancer, chronic lymphocytic leukemia, colorectal cancer, pancreatic cancer, esophageal cancer, hepatocellular cancer, lymphoblastic leukemia including acute lymphoblastic leukemia, follicular lymphoma, lymphoid malignancies of T-cell or B-cell origin, metastatic castration resistant prostate cancer, bladder urothelial carcinoma, melanoma, myelogenous leukemia, myeloma, oral cancer, ovarian cancer, non-small cell lung cancer, prostate cancer, small cell lung cancer, spleen cancer, or head and neck cancer.

In some embodiments, the disclosed ADCs may be administered in any cell or tissue that expresses EphA2, such as a EphA2-expressing cancer cell or tissue. An exemplary embodiment includes a method of killing a EphA2-expressing cancer cell or tissue. The method may be used with any cell or tissue that expresses EphA2, such as a cancerous cell or a metastatic lesion. Non-limiting examples of EphA2-expressing cancers include breast cancer, non-small cell lung cancer, pancreatic cancer, esophageal cancer, head and neck cancer, gastric or stomach cancer, bladder cancer, and colorectal cancer.

In some embodiments, the disclosed ADCs may be administered in any cell or tissue that expresses B7-H3 (CD276), such as a B7-H3 (CD276)-expressing cancer cell or tissue. An exemplary embodiment includes a method of killing a B7-H3 (CD276)-expressing cancer cell or tissue. The method may be used with any cell or tissue that expresses B7-H3 (CD276), such as a cancerous cell or a metastatic lesion. Non-limiting examples of B7-H3 (CD276)-expressing cancers include colorectal cancer, pancreatic cancer, lymphoma, non-small cell lung cancer, small cell lung cancer, breast cancer including ER positive breast cancer, metastatic castration resistant prostate cancer, melanoma, bladder urothelial carcinoma, head and neck cancer, and leukemia (e.g., acute myeloid leukemia).

Exemplary methods include the steps of contacting a cell with an ADC, as described herein, in an effective amount, i.e., an amount sufficient to kill the cell. The method can be used on cells in culture, e.g., in vitro, in vivo, ex vivo, or in situ. For example, cells that express EphA2 (e.g., cells collected by biopsy of a tumor or metastatic lesion; cells from an established cancer cell line; or recombinant cells), can be cultured in vitro in culture medium and the contacting step can be affected by adding the ADC to the culture medium. The method will result in killing of cells expressing EphA2, including in particular cancer cells expressing EphA2. Alternatively, the ADC can be administered to a subject by any suitable administration route (e.g., intravenous, subcutaneous, or direct contact with a tumor tissue) to have an effect in vivo. This approach can be used for antibodies targeting other cell surface antigens (e.g., B7-H3 (CD276)).

The in vivo effect of a disclosed ADC therapeutic composition can be evaluated in a suitable animal model. For example, xenogeneic cancer models can be used, wherein cancer explants or passaged xenograft tissues are introduced into immune compromised animals, such as nude or SCID mice (Klein et al. (1997) Nature Med. 3:402-8). Efficacy may be predicted using assays that measure inhibition of tumor formation, tumor regression or metastasis, and the like.

In vivo assays that evaluate the promotion of tumor death by mechanisms such as apoptosis may also be used. In some embodiments, xenografts from tumor bearing mice treated with the therapeutic composition can be examined for the presence of apoptotic foci and compared to untreated control xenograft-bearing mice. The extent to which apoptotic foci are found in the tumors of the treated mice provides an indication of the therapeutic efficacy of the composition.

Further provided herein are methods of treating a disorder, e.g., a cancer. The compositions described herein, e.g., the ADCs disclosed herein, can be administered to a non-human mammal or human subject for therapeutic purposes. The therapeutic methods include administering to a subject having or suspected of having a cancer a therapeutically effective amount of a composition comprising an panRAS inhibitor, e.g., an ADC where the inhibitor is linked to a targeting antibody that binds to an antigen (1) expressed on a cancer cell, (2) is accessible to binding, and/or (3) is localized or predominantly expressed on a cancer cell surface as compared to a non-cancer cell.

An exemplary embodiment is a method of treating a subject having or suspected of having a cancer, comprising administering to the subject a therapeutically effective amount of a composition disclosed herein, e.g., an ADC, composition, or pharmaceutical composition (e.g., any of the exemplary ADCs, compositions, or pharmaceutical compositions disclosed herein). In some embodiments, the cancer expresses a target antigen. In some embodiments, the target antigen is BCMA, CD33, HER2, CD38, CD48, CD79b, PCAD, CD74, CD138, SLAMF7, CD123, CLL1, FLT3, CD7, CKIT, CD56, DLL3, DLK1, B7-H3, B7-H4, EGFR, CD71, EPCAM, FOLR1, ENPP3, MET, AXL, SLC34A2 (NaPi2b), Nectin4, TROP2, LIV1, CD46, MSLN, CD142 (F3), MUC1, MUC16, SLC39A6, TFRC, TACSTD2, GPNMB, EphA2, CD56, SEZ6, CD25, CCR8, CEACAM5, CEACAM6, 4-1 BB, 5AC, 5T4, Alpha-fetoprotein, angiopoietin 2, ASLG659, TCLI, BMPRIB, Brevican BCAN, BEHAB, C242 antigen, C5, CA-125, CA-125 (imitation), CA-IX (Carbonic anhydrase 9), CCR4, CD140a, CD152, CD19, CD20, CD200, CD21 (C3DR) I), CD22 (B-cell receptor CD22-B isoform), CD221, CD23 (gE receptor), CD28, CD30 (TNFRSF8), CD37, CD4, CD40, CD44 v6, CD51, CD52, CD70, CD72 (Lyb-2, B-cell differentiation antigen CD72), CD79a, CD80, CD166 (ALCAM), CDH17, CA9, CEA, CEA-related antigen, ch4D5, CLDN18.2, CRIPTO (CR, CRI, CRGF, TDGF1, CFC1B), CTLA-4, CXCR5, DLL4, DR5, E16 (LATI, SLC7A5), EGFL7, EphB2R (DRT, ERK, Hek5, EPHT3, Tyro5), Episialin, ERBB3, ETBR (Endothelin type B receptor), FCRHI (Fc receptor-like protein I), FcRH2 (IFGP4, IRTA4, SPAPI, SPAP IB, SPAP IC), Fibronectin extra domain-B, Frizzled receptor, GD2, GD3 ganglioside, GEDA, HER1, HER2/neu, HER3, HGF, HLA-DOB, HLA-DR, Human scatter factor receptor kinase, IGF-I receptor, IL-13, IL20R (ZCYTOR7), IL-6, ILGF2, ILFRIR, integrin u, IRTA2 (Immunoglobulin superfamily receptor translocation associated 2), Lewis-Y antigen, LY64 (RP105), LY6E, STEAP1, ADAM9, PTK7, MMP14, TM4SF1, ITGB6, FXYD5, MCP-I, MDP (DPEPI), MPF, MSLN, SMR, mesothelin, megakaryocyte, PD-I, PDCDI, PDGF-R u, Prostate specific membrane antigen (PSMA), PSCA (Prostate stem cell antigen precursor), PRLR (Prolactin Receptor), PSCA hlg, RANKL, RON, SDCI, Sema Sb, STEAP I, STEAP2, PCANAP I, STAMP I, STEAP2, STMP, prostate cancer associated gene I, TAG-72, TEMI, Tenascin C, TENB2, (TMEFF2, tomoregulin, TPEF, HPPI, TR), TGF-IJ, TRAIL-E2, TRAIL-RI, TRAIL-R2, T17M4 (BR22450, FLJ20041, TRPM4, TRPM4B, transient receptor potential cation channel subfamily M, member 4), TWEAK-R, TYRP I (glycoprotein 75), VEGF, VEGF-A, EGFR-I, VEGFR-2, or Vimentin. In some embodiments, the target antigen is EphA2 or B7-H3 (CD276). In some embodiments, the cancer is a tumor or a hematological cancer. In some embodiments, the cancer is a breast cancer including ER positive breast cancer, multiple myeloma, plasma cell myeloma, leukemia, lymphoma, sarcoma, gastric or stomach cancer, acute myeloid leukemia, bladder cancer, brain cancer, bone marrow cancer, cervical cancer, chronic lymphocytic leukemia, colorectal cancer, pancreatic cancer, esophageal cancer, hepatocellular cancer, lymphoblastic leukemia including acute lymphoblastic leukemia, follicular lymphoma, lymphoid malignancies of T-cell or B-cell origin, metastatic castration resistant prostate cancer, bladder urothelial carcinoma, melanoma, myelogenous leukemia, myeloma, oral cancer, ovarian cancer, non-small cell lung cancer, prostate cancer, small cell lung cancer, spleen cancer, or head and neck cancer.

Another exemplary embodiment is a method of delivering a panRAS inhibitor to a cell expressing EphA2, comprising conjugating the panRAS inhibitor to an antibody or antigen-binding fragment that immunospecifically binds to a EphA2 epitope and exposing the cell to the ADC. Exemplary cancer cells that express EphA2 for which the ADCs of the present disclosure are indicated include breast cancer, non-small cell lung cancer, pancreatic cancer, esophageal cancer, head and neck cancer, gastric or stomach cancer, bladder cancer, and colorectal cancer cells.

Another exemplary embodiment is a method of delivering a panRAS inhibitor to a cell expressing B7-H3 (CD276), comprising conjugating the panRAS inhibitor to an antibody or antigen-binding fragment that immunospecifically binds to a B7-H3 (CD276) epitope and exposing the cell to the ADC. Exemplary cancer cells that express B7-H3 (CD276) for which the ADCs of the present disclosure are indicated include colorectal cancer, pancreatic cancer, lymphoma, and leukemia cells.

In certain aspects, the present disclosure further provides methods of reducing or inhibiting growth of a tumor (e.g., an EphA2-expressing tumor, a B7-H3 (CD276)-expressing tumor), comprising administering a therapeutically effective amount of an ADC or composition comprising an ADC. In some embodiments, the treatment is sufficient to reduce or inhibit the growth of the patient's tumor, reduce the number or size of metastatic lesions, reduce tumor load, reduce primary tumor load, reduce invasiveness, prolong survival time, and/or maintain or improve the quality of life. In some embodiments, the tumor is resistant or refractory to treatment with the antibody or antigen-binding fragment of the ADC (e.g., an anti-EphA2 antibody or antigen-binding fragment, an anti-B7-H3 (CD276) antibody or antigen-binding fragment) when administered alone, and/or the tumor is resistant or refractory to treatment with the panRAS inhibitor drug moiety when administered alone.

An exemplary embodiment is a method of reducing or inhibiting the growth of a tumor in a subject, comprising administering to the subject a therapeutically effective amount of an ADC, composition, or pharmaceutical composition (e.g., any of the exemplary ADCs, compositions, or pharmaceutical compositions disclosed herein). In some embodiments, the tumor expresses a target antigen. In some embodiments, the target antigen is BCMA, CD33, HER2, CD38, CD48, CD79b, PCAD, CD74, CD138, SLAMF7, CD123, CLL1, FLT3, CD7, CKIT, CD56, DLL3, DLK1, B7-H3, B7-H4, EGFR, CD71, EPCAM, FOLR1, ENPP3, MET, AXL, SLC34A2 (NaPi2b), Nectin4, TROP2, LIV1, CD46, MSLN, CD142 (F3), MUC1, MUC16, SLC39A6, TFRC, TACSTD2, GPNMB, EphA2, CD56, SEZ6, CD25, CCR8, CEACAM5, CEACAM6, 4-1BB, 5AC, 5T4, Alpha-fetoprotein, angiopoietin 2, ASLG659, TCLI, BMPRIB, Brevican BCAN, BEHAB, C242 antigen, C5, CA-125, CA-125 (imitation), CA-IX (Carbonic anhydrase 9), CCR4, CD140a, CD152, CD19, CD20, CD200, CD21 (C3DR) I), CD22 (B-cell receptor CD22-B isoform), CD221, CD23 (gE receptor), CD28, CD30 (TNFRSF8), CD37, CD4, CD40, CD44 v6, CD51, CD52, CD70, CD72 (Lyb-2, B-cell differentiation antigen CD72), CD79a, CD80, CD166 (ALCAM), CDH17, CA9, CEA, CEA-related antigen, ch4D5, CLDN18.2, CRIPTO (CR, CRI, CRGF, TDGF1, CFC1B), CTLA-4, CXCR5, DLL4, DR5, E16 (LATI, SLC7A5), EGFL7, EphB2R (DRT, ERK, Hek5, EPHT3, Tyro5), Episialin, ERBB3, ETBR (Endothelin type B receptor), FCRHI (Fc receptor-like protein I), FcRH2 (IFGP4, IRTA4, SPAPI, SPAP IB, SPAP IC), Fibronectin extra domain-B, Frizzled receptor, GD2, GD3 ganglioside, GEDA, HER1, HER2/neu, HER3, HGF, HLA-DOB, HLA-DR, Human scatter factor receptor kinase, IGF-I receptor, IL-13, IL20R (ZCYTOR7), IL-6, ILGF2, ILFRIR, integrin u, IRTA2 (Immunoglobulin superfamily receptor translocation associated 2), Lewis-Y antigen, LY64 (RP105), LY6E, STEAP1, ADAM9, PTK7, MMP14, TM4SF1, ITGB6, FXYD5, MCP-I, MDP (DPEPI), MPF, MSLN, SMR, mesothelin, megakaryocyte, PD-I, PDCDI, PDGF-R u, Prostate specific membrane antigen (PSMA), PSCA (Prostate stem cell antigen precursor), PRLR (Prolactin Receptor), PSCA hlg, RANKL, RON, SDCI, Sema Sb, STEAP I, STEAP2, PCANAP I, STAMP I, STEAP2, STMP, prostate cancer associated gene I, TAG-72, TEMI, Tenascin C, TENB2, (TMEFF2, tomoregulin, TPEF, HPPI, TR), TGF-IJ, TRAIL-E2, TRAIL-RI, TRAIL-R2, T17M4 (BR22450, FLJ20041, TRPM4, TRPM4B, transient receptor potential cation channel subfamily M, member 4), TWEAK-R, TYRP I (glycoprotein 75), VEGF, VEGF-A, EGFR-I, VEGFR-2, or Vimentin. In some embodiments, the target antigen is EphA2 or B7-H3 (CD276). In some embodiments, the tumor is a breast cancer including ER positive breast cancer, multiple myeloma, plasma cell myeloma, leukemia, lymphoma, sarcoma, gastric or stomach cancer, acute myeloid leukemia, bladder cancer, brain cancer, bone marrow cancer, cervical cancer, chronic lymphocytic leukemia, colorectal cancer, pancreatic cancer, esophageal cancer, hepatocellular cancer, iymphoblastic leukemia including acute lymphoblastic leukemia, follicular lymphoma, lymphoid malignancies of T-cell or B-cell origin, metastatic castration resistant prostate cancer, bladder urothelial carcinoma, melanoma, myelogenous leukemia, myeloma, oral cancer, ovarian cancer, non-small cell lung cancer, prostate cancer, small cell lung cancer, spleen cancer, or head and neck cancer. In some embodiments, the tumor is a gastric cancer. In some embodiments, administration of the ADC, composition, or pharmaceutical composition reduces or inhibits the growth of the tumor by at least about 10%, at least about 20%, at least about 30%, at least about 40%, at least about 50%, at least about 60%, at least about 70%, at least about 80%, at least about 90%, at least about 95%, or at least about 99%, as compared to growth in the absence of treatment.

Another exemplary embodiment is a method of delaying or slowing the growth of a tumor in a subject, comprising administering to the subject a therapeutically effective amount of an ADC, composition, or pharmaceutical composition (e.g., any of the exemplary ADCs, compositions, or pharmaceutical compositions disclosed herein). In some embodiments, the target antigen is EphA2 or B7-H3 (CD276). In some embodiments, the tumor is a breast cancer including ER positive breast cancer, multiple myeloma, plasma cell myeloma, leukemia, lymphoma, sarcoma, gastric or stomach cancer, acute myeloid leukemia, bladder cancer, brain cancer, bone marrow cancer, cervical cancer, chronic lymphocytic leukemia, colorectal cancer, pancreatic cancer, esophageal cancer, hepatocellular cancer, lymphoblastic leukemia including acute lymphoblastic leukemia, follicular lymphoma, lymphoid malignancies of T-cell or B-cell origin, metastatic castration resistant prostate cancer, bladder urothelial carcinoma, melanoma, myelogenous leukemia, myeloma, oral cancer, ovarian cancer, non-small cell lung cancer, prostate cancer, small cell lung cancer, spleen cancer, or head and neck cancer. In some embodiments, the tumor is a gastric cancer. In some embodiments, administration of the ADC, composition, or pharmaceutical composition delays or slows the growth of the tumor by at least about 10%, at least about 20%, at least about 30%, at least about 40%, at least about 50%, at least about 60%, at least about 70%, at least about 80%, at least about 90%, at least about 95%, or at least about 99%, as compared to growth in the absence of treatment.

In certain aspects, the present disclosure further provides methods of reducing or slowing the expansion of a cancer cell population (e.g., a EphA2-expressing cancer cell population, a B7-H3 (CD276)-expressing cancer cell population), comprising administering a therapeutically effective amount of an ADC or composition comprising an ADC.

An exemplary embodiment is a method of reducing or slowing the expansion of a cancer cell population in a subject, comprising administering to the subject a therapeutically effective amount of an ADC, composition, or pharmaceutical composition (e.g., any of the exemplary ADCs, compositions, or pharmaceutical compositions disclosed herein). In some embodiments, the target antigen is EphA2 or B7-H3 (CD276). In some embodiments, the cancer cell population is from a tumor or a hematological cancer. In some embodiments, the cancer cell population is a breast cancer including ER positive breast cancer, multiple myeloma, plasma cell myeloma, leukemia, lymphoma, sarcoma, gastric or stomach cancer, acute myeloid leukemia, bladder cancer, brain cancer, bone marrow cancer, cervical cancer, chronic lymphocytic leukemia, colorectal cancer, pancreatic cancer, esophageal cancer, hepatocellular cancer, lymphoblastic leukemia including acute lymphoblastic leukemia, follicular lymphoma, lymphoid malignancies of T-cell or B-cell origin, metastatic castration resistant prostate cancer, bladder urothelial carcinoma, melanoma, myelogenous leukemia, myeloma, oral cancer, ovarian cancer, non-small cell lung cancer, prostate cancer, small cell lung cancer, spleen cancer, or head and neck cancer. In some embodiments, administration of the ADC, composition, or pharmaceutical composition reduces the cancer cell population by at least about 10%, at least about 20%, at least about 30%, at least about 40%, at least about 50%, at least about 60%, at least about 70%, at least about 80%, at least about 90%, at least about 95%, or at least about 99%, as compared to the population in the absence of treatment. In some embodiments, administration of the ADC, composition, or pharmaceutical composition slows the expansion of the cancer cell population by at least about 10%, at least about 20%, at least about 30%, at least about 40%, at least about 50%, at least about 60%, at least about 70%, at least about 80%, at least about 90%, at least about 95%, or at least about 99%, as compared to expansion in the absence of treatment.

Also provided herein are methods of determining whether a subject having or suspected of having a cancer will be responsive to treatment with the disclosed ADCs and compositions. An exemplary embodiment is a method of determining whether a subject having or suspected of having a cancer will be responsive to treatment with an ADC, composition, or pharmaceutical composition (e.g., any of the exemplary ADCs, compositions, or pharmaceutical compositions disclosed herein) by providing a biological sample from the subject; contacting the sample with the ADC; and detecting binding of the ADC to cancer cells in the sample. In some embodiments, the sample is a tissue biopsy sample, a blood sample, or a bone marrow sample. In some embodiments, the method comprises providing a biological sample from the subject; contacting the sample with the ADC; and detecting one or more markers of cancer cell death in the sample (e.g., increased expression of one or more apoptotic markers, reduced expansion of a cancer cell population in culture, etc.).

Further provided herein are therapeutic uses of the disclosed ADCs and compositions. An exemplary embodiment is an ADC, composition, or pharmaceutical composition (e.g., any of the exemplary ADCs, compositions, or pharmaceutical compositions disclosed herein) for use in treating a subject having or suspected of having a cancer (e.g., an EphA2-expressing cancer, a B7-H3 (CD276)-expressing cancer). Another exemplary embodiment is a use of an ADC, composition, or pharmaceutical composition (e.g., any of the exemplary ADCs, compositions, or pharmaceutical compositions disclosed herein) in treating a subject having or suspected of having a cancer (e.g., an EphA2-expressing cancer, a B7-H3 (CD276)-expressing cancer). Another exemplary embodiment is a use of an ADC, composition, or pharmaceutical composition (e.g., any of the exemplary ADCs, compositions, or pharmaceutical compositions disclosed herein) in a method of manufacturing a medicament for treating a subject having or suspected of having a cancer (e.g., an EphA2-expressing cancer, a B7-H3 (CD276)-expressing cancer). Methods for identifying subjects having cancers that express a target antigen (e.g., EphA2 or B7-H3 (CD276)) are known in the art and may be used to identify suitable patients for treatment with a disclosed ADC compound or composition.

Moreover, ADCs of the present disclosure may be administered to a non-human mammal expressing an antigen with which the ADC is capable of binding for veterinary purposes or as an animal model of human disease. Regarding the latter, such animal models may be useful for evaluating the therapeutic efficacy of the disclosed ADCs (e.g., testing of dosages and time courses of administration).

The therapeutic compositions used in the practice of the foregoing methods may be formulated into pharmaceutical compositions comprising a pharmaceutically acceptable carrier suitable for the desired delivery method. An exemplary embodiment is a pharmaceutical composition comprising an ADC of the present disclosure and a pharmaceutically acceptable carrier, e.g., one suitable for a chosen means of administration, e.g., intravenous administration. The pharmaceutical composition may also comprise one or more additional inactive and/or therapeutic agents that are suitable for treating or preventing, for example, a cancer (e.g., a standard-of-care agent, etc.). The pharmaceutical composition may also comprise one or more carrier, excipient, and/or stabilizer components, and the like. Methods of formulating such pharmaceutical compositions and suitable formulations are known in the art (see, e.g., “Remington's Pharmaceutical Sciences,” Mack Publishing Co., Easton, PA).

Suitable carriers include any material that, when combined with the therapeutic composition, retains the anti-tumor function of the therapeutic composition and is generally non-reactive with the patient's immune system. Pharmaceutically acceptable carriers include any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol, mesylate salt, and the like, as well as combinations thereof. In many cases, isotonic agents are included, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the ADC.

A pharmaceutical composition of the present disclosure can be administered by a variety of methods known in the art. The route and/or mode of administration may vary depending upon the desired results. In some embodiments, the therapeutic formulation is solubilized and administered via any route capable of delivering the therapeutic composition to the cancer site. Potentially effective routes of administration include, but are not limited to, parenteral (e.g., intravenous, subcutaneous), intraperitoneal, intramuscular, intratumor, intradermal, intraorgan, orthotopic, and the like. In some embodiments, the administration is intravenous, subcutaneous, intraperitoneal, or intramuscular. The pharmaceutically acceptable carrier should be suitable for the route of administration, e.g., intravenous or subcutaneous administration (e.g., by injection or infusion). Depending on the route of administration, the active compound(s), i.e., the ADC and/or any additional therapeutic agent, may be coated in a material to protect the compound(s) from the action of acids and other natural conditions that may inactivate the compound(s). Administration can be either systemic or local.

The therapeutic compositions disclosed herein may be sterile and stable under the conditions of manufacture and storage, and may be in a variety of forms. These include, for example, liquid, semi-solid, and solid dosage forms, such as liquid solutions (e.g., injectable and infusible solutions), dispersions or suspensions, tablets, pills, powders, liposomes, and suppositories. The form depends on the intended mode of administration and therapeutic application. In some embodiments, the disclosed ADCs can be incorporated into a pharmaceutical composition suitable for parenteral administration. The injectable solution may be composed of either a liquid or lyophilized dosage form in a flint or amber vial, ampule, or pre-filled syringe, or other known delivery or storage device. In some embodiments, one or more of the ADCs or pharmaceutical compositions is supplied as a dry sterilized lyophilized powder or water free concentrate in a hermetically sealed container and can be reconstituted (e.g., with water or saline) to the appropriate concentration for administration to a subject.

Typically, a therapeutically effective amount or efficacious amount of a disclosed composition, e.g., a disclosed ADC, is employed in the pharmaceutical compositions of the present disclosure. The composition, e.g., one comprising an ADC, may be formulated into a pharmaceutically acceptable dosage form by conventional methods known in the art. Dosages and administration protocols for the treatment of cancers using the foregoing methods will vary with the method and the target cancer, and will generally depend on a number of other factors appreciated in the art.

Dosage regimens for compositions disclosed herein, e.g., those comprising ADCs alone or in combination with at least one additional inactive and/or active therapeutic agent, may be adjusted to provide the optimum desired response (e.g., a therapeutic response). For example, a single bolus of one or both agents may be administered at one time, several divided doses may be administered over a predetermined period of time, or the dose of one or both agents may be proportionally increased or decreased as indicated by the exigencies of the therapeutic situation. In some embodiments, treatment involves single bolus or repeated administration of the ADC preparation via an acceptable route of administration. In some embodiments, the ADC is administered to the patient daily, weekly, monthly, or any time period in between. For any particular subject, specific dosage regimens may be adjusted over time according to the individual's need, and the professional judgment of the treating clinician. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Dosage values for compositions comprising an ADC and/or any additional therapeutic agent(s), may be selected based on the unique characteristics of the active compound(s), and the particular therapeutic effect to be achieved. A physician or veterinarian can start doses of the ADC employed in the pharmaceutical composition at levels lower than that required to achieve the desired therapeutic effect and gradually increase the dosage until the desired effect is achieved. In general, effective doses of the compositions of the present disclosure, for the treatment of a cancer may vary depending upon many different factors, including means of administration, target site, physiological state of the patient, whether the patient is human or an animal, other medications administered, and whether treatment is prophylactic or therapeutic. The selected dosage level may also depend upon a variety of pharmacokinetic factors including the activity of the particular compositions of the present disclosure employed, or the ester, salt, or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors. Treatment dosages may be titrated to optimize safety and efficacy.

Toxicity and therapeutic efficacy of compounds provided herein can be determined by standard pharmaceutical procedures in cell culture or in animal models. For example, LD50, ED50, EC50, and IC50 may be determined, and the dose ratio between toxic and therapeutic effects (LD50/ED50) may be calculated as the therapeutic index. The data obtained from in vitro and in vivo assays can be used in estimating or formulating a range of dosage for use in humans. For example, the compositions and methods disclosed herein may initially be evaluated in xenogeneic cancer models (e.g., an NCI-H929 multiple myeloma mouse model).

In some embodiments, an ADC or composition comprising an ADC is administered on a single occasion. In other embodiments, an ADC or composition comprising an ADC is administered on multiple occasions. Intervals between single dosages can be, e.g., daily, weekly, monthly, or yearly. Intervals can also be irregular, based on measuring blood levels of the administered agent (e.g., the ADC) in the patient in order to maintain a relatively consistent plasma concentration of the agent. The dosage and frequency of administration of an ADC or composition comprising an ADC may also vary depending on whether the treatment is prophylactic or therapeutic. In prophylactic applications, a relatively low dosage may be administered at relatively infrequent intervals over a long period of time. Some patients continue to receive treatment for the rest of their lives. In therapeutic applications, a relatively higher dosage at relatively shorter intervals is sometimes required until progression of the disease is reduced or terminated, and preferably until the patient shows partial or complete amelioration of one or more symptoms of disease. Thereafter, the patient may be administered a lower, e.g., prophylactic regime.

The above therapeutic approaches can be combined with any one of a wide variety of additional surgical, chemotherapy, or radiation therapy regimens. In some embodiments, the ADCs or compositions disclosed herein are co-formulated and/or co-administered with one or more additional therapeutic agents, e.g., one or more chemotherapeutic agents, one or more standard-of-care agents for the particular condition being treated.

Kits for use in the therapeutic and/or diagnostic applications described herein are also provided. Such kits may comprise a carrier, package, or container that is compartmentalized to receive one or more containers such as vials, tubes, and the like, each of the container(s) comprising one of the separate elements to be used in a method disclosed herein. A label may be present on or with the container(s) to indicate that an ADC or composition within the kit is used for a specific therapy or non-therapeutic application, such as a prognostic, prophylactic, diagnostic, or laboratory application. A label may also indicate directions for either in vivo or in vitro use, such as those described herein. Directions and or other information may also be included on an insert(s) or label(s), which is included with or on the kit. The label may be on or associated with the container. A label may be on a container when letters, numbers, or other characters forming the label are molded or etched into the container itself. A label may be associated with a container when it is present within a receptacle or carrier that also holds the container, e.g., as a package insert. The label may indicate that an ADC or composition within the kit is used for diagnosing or treating a condition, such as a cancer a described herein.

In some embodiments, a kit comprises an ADC or composition comprising an ADC. In some embodiments, the kit further comprises one or more additional components, including but not limited to: instructions for use; other reagents, e.g., a therapeutic agent (e.g., a standard-of-care agent); devices, containers, or other materials for preparing the ADC for administration; pharmaceutically acceptable carriers; and devices, containers, or other materials for administering the ADC to a subject. Instructions for use can include guidance for therapeutic applications including suggested dosages and/or modes of administration, e.g., in a patient having or suspected of having a cancer. In some embodiments, the kit comprises an ADC and instructions for use of the ADC in treating, preventing, and/or diagnosing a cancer.

It is known that elevated panRAS (e.g., K-Ras (including splice variants KRAS4A and KRAS4B), H-Ras and N-Ras) expression correlates with resistance to radiation therapy and chemotherapy. Antibody-drug conjugates (ADCs) that may not be sufficiently effective as monotherapy to treat cancer can be administered in combination with other therapeutic agents (including non-targeted and targeted therapeutic agents) or radiation therapy (including radioligand therapy) to provide therapeutic benefit. Without wishing to be bound by theory, it is believed that the ADCs described herein sensitize tumor cells to the treatment with other therapeutic agents (including standard of care chemotherapeutic agents to which the tumor cells may have developed resistance) and/or radiation therapy. In some embodiments, antibody drug conjugates described herein, are administered to a subject having cancer in an amount effective to sensitize the tumor cells. As used herein, the term “sensitize” means that the treatment with ADC increases the potency or efficacy of the treatment with other therapeutic agents and/or radiation therapy against tumor cells.

### Combination Therapies

In some embodiments, the present disclosure provides methods of treatment wherein the antibody-drug conjugates disclosed herein are administered in combination with one or more (e.g., 1 or 2) additional therapeutic agents. Exemplary combination partners are disclosed herein.

In certain embodiments, a combination described herein comprises a PD-1 inhibitor. In some embodiments, the PD-1 inhibitor is chosen from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune). In some embodiments, the PD-1 inhibitor is PDR001. PDR001 is also known as Spartalizumab.

In certain embodiments, a combination described herein comprises a LAG-3 inhibitor. In some embodiments, the LAG-3 inhibitor is chosen from LAG525 (Novartis), BMS-986016 (Bristol-Myers Squibb), or TSR-033 (Tesaro).

In certain embodiments, a combination described herein comprises a TIM-3 inhibitor. In some embodiments, the TIM-3 inhibitor is MBG453 (Novartis), TSR-022 (Tesaro), LY-3321367 (Eli Lily), Sym23 (Symphogen), BGB-A425 (Beigene), INCAGN-2390 (Agenus), BMS-986258 (BMS), RO-7121661 (Roche), or LY-3415244 (Eli Lilly).

In certain embodiments, a combination described herein comprises a PDL1 inhibitor. In one embodiment, the PDL1 inhibitor is chosen from FAZ053 (Novartis), atezolizumab (Genentech), durvalumab (Astra Zeneca), or avelumab (Pfizer).

In certain embodiments, a combination described herein comprises a GITR agonist. In some embodiments, the GITR agonist is chosen from GWN323 (NVS), BMS-986156, MK-4166 or MK-1248 (Merck), TRX518 (Leap Therapeutics), INCAGN1876 (Incyte/Agenus), AMG 228 (Amgen) or INBRX-110 (Inhibrx).

In some embodiments, a combination described herein comprises an IAP inhibitor. In some embodiments, the IAP inhibitor comprises LCL161 or a compound disclosed in International Application Publication No. WO 2008/016893.

In an embodiment, the combination comprises an mTOR inhibitor, e.g., RAD001 (also known as everolimus).

In an embodiment, the combination comprises a HDAC inhibitor, e.g., LBH589. LBH589 is also known as panobinostat.

In an embodiment, the combination comprises an IL-17 inhibitor, e.g., CJM112.

In certain embodiments, a combination described herein comprises an estrogen receptor (ER) antagonist. In some embodiments, the estrogen receptor antagonist is used in combination with a PD-1 inhibitor, a CDK4/6 inhibitor, or both. In some embodiments, the combination is used to treat an ER positive (ER+) cancer or a breast cancer (e.g., an ER+ breast cancer).

In some embodiments, the estrogen receptor antagonist is a selective estrogen receptor degrader (SERD). SERDs are estrogen receptor antagonists which bind to the receptor and result in e.g., degradation or down-regulation of the receptor (Boer K. et al., (2017) Therapeutic Advances in Medical Oncology 9(7): 465-479). ER is a hormone-activated transcription factor important for e.g., the growth, development and physiology of the human reproductive system. ER is activated by, e.g., the hormone estrogen (17beta estradiol). ER expression and signaling is implicated in cancers (e.g., breast cancer), e.g., ER positive (ER+) breast cancer. In some embodiments, the SERD is chosen from LSZ102, fulvestrant, brilanestrant, or elacestrant.

In some embodiments, the SERD comprises a compound disclosed in International Application Publication No. WO 2014/130310, which is hereby incorporated by reference in its entirety.

In some embodiments, the SERD comprises LSZ102. LSZ102 has the chemical name: (E)-3-(4-((2-(2-(1,1-difluoroethyl)-4-fluorophenyl)-6-hydroxybenzo[b]thiophen-3-yl)oxy)phenyl)acrylic acid. In some embodiments, the SERD comprises fulvestrant (CAS Registry Number: 129453-61-8), or a compound disclosed in International Application Publication No. WO 2001/051056, which is hereby incorporated by reference in its entirety. In some embodiments, the SERD comprises elacestrant (CAS Registry Number: 722533-56-4), or a compound disclosed in U.S. Pat. No. 7,612,114, which is incorporated by reference in its entirety. Elacestrant is also known as RAD1901, ER-306323 or (6R)-6-{2-[Ethyl({4-[2-(ethylamino)ethyl]phenyl}methyl)amino]-4-methoxyphenyl}-5,6,7,8-tetrahydronaphthalen-2-ol. Elacestrant is an orally bioavailable, non-steroidal combined selective estrogens receptor modulator (SERM) and a SERD. Elacestrant is also disclosed, e.g., in Garner F et al., (2015) Anticancer Drugs 26(9):948-56. In some embodiments, the SERD is brilanestrant (CAS Registry Number: 1365888-06-7), or a compound disclosed in International Application Publication No. WO 2015/136017, which is incorporated by reference in its entirety.

In some embodiments, the SERD is chosen from RU 58668, GW7604, AZD9496, bazedoxifene, pipendoxifene, arzoxifene, OP-1074, or acolbifene, e.g., as disclosed in McDonell et al. (2015) Journal of Medicinal Chemistry 58(12) 4883-4887.

Other exemplary estrogen receptor antagonists are disclosed, e.g., in WO 2011/156518, WO 2011/159769, WO 2012/037410, WO 2012/037411, and US 2012/0071535, all of which are hereby incorporated by reference in their entirety.

In certain embodiments, a combination described herein comprises an inhibitor of Cyclin-Dependent Kinases 4 or 6 (CDK4/6). In some embodiments, the CDK4/6 inhibitor is used in combination with a PD-1 inhibitor, an estrogen receptor (ER) antagonist, or both. In some embodiments, the combination is used to treat an ER positive (ER+) cancer or a breast cancer (e.g., an ER+ breast cancer). In some embodiments, the CDK4/6 inhibitor is chosen from ribociclib, abemaciclib (Eli Lilly), or palbociclib.

In some embodiments, the CDK4/6 inhibitor comprises ribociclib (CAS Registry Number: 1211441-98-3), or a compound disclosed in U.S. Pat. Nos. 8,415,355 and 8,685,980, which are incorporated by reference in their entirety.

In some embodiments, the CDK4/6 inhibitor comprises a compound disclosed in International Application Publication No. WO 2010/020675 and U.S. Pat. Nos. 8,415,355 and 8,685,980, which are incorporated by reference in their entirety.

In some embodiments, the CDK4/6 inhibitor comprises ribociclib (CAS Registry Number: 1211441-98-3). Ribociclib is also known as LEE011, KISQALI®, or 7-cyclopentyl-N,N-dimethyl-2-((5-(piperazin-1-yl)pyridin-2-yl)amino)-7H-pyrrolo[2,3-d]pyrimidine-6-carboxamide.

In some embodiments, the CDK4/6 inhibitor comprises abemaciclib (CAS Registry Number: 1231929-97-7). Abemaciclib is also known as LY835219 or N-[5-[(4-Ethyl-1-piperazinyl)methyl]-2-pyridinyl]-5-fluoro-4-[4-fluoro-2-methyl-1-(1-methylethyl)-1H-benzimidazol-6-yl]-2-pyrimidinamine. Abemaciclib is a CDK inhibitor selective for CDK4 and CDK6 and is disclosed, e.g., in Torres-Guzman R et al. (2017) Oncotarget 10.18632/oncotarget.17778.

In some embodiments, the CDK4/6 inhibitor comprises palbociclib (CAS Registry Number: 571190-30-2). Palbociclib is also known as PD-0332991, IBRANCE® or 6-Acetyl-8-cyclopentyl-5-methyl-2-{[5-(1-piperazinyl)-2-pyridinyl]amino}pyrido[2,3-d]pyrimidin-7(8H)-one. Palbociclib inhibits CDK4 with an IC50 of 11 nM, and inhibits CDK6 with an IC50 of 16 nM, and is disclosed, e.g., in Finn et al. (2009) Breast Cancer Research 11(5):R77.

In certain embodiments, a combination described herein comprises an inhibitor of chemokine (C-X-C motif) receptor 2 (CXCR2). In some embodiments, the CXCR2 inhibitor is chosen from 6-chloro-3-((3,4-dioxo-2-(pentan-3-ylamino)cyclobut-1-en-1-yl)amino)-2-hydroxy-N-methoxy-N-methylbenzenesulfonamide, danirixin, reparixin, or navarixin.

In some embodiments, the CSF-1/1R binding agent is chosen from an inhibitor of macrophage colony-stimulating factor (M-CSF), e.g., a monoclonal antibody or Fab to M-CSF (e.g., MCS110), a CSF-1R tyrosine kinase inhibitor (e.g., 4-((2-(((1R,2R)-2-hydroxycyclohexyl)amino)benzo[d]thiazol-6-yl)oxy)-N-methylpicolinamide or BLZ945), a receptor tyrosine kinase inhibitor (RTK) (e.g., pexidartinib), or an antibody targeting CSF-1R (e.g., emactuzumab or FPA008). In some embodiments, the CSF-1/1R inhibitor is BLZ945. In some embodiments, the CSF-1/1R binding agent is MCS110. In other embodiments, the CSF-1/1R binding agent is pexidartinib.

In certain embodiments, a combination described herein comprises a c-MET inhibitor. c-MET, a receptor tyrosine kinase overexpressed or mutated in many tumor cell types, plays key roles in tumor cell proliferation, survival, invasion, metastasis, and tumor angiogenesis. Inhibition of c-MET may induce cell death in tumor cells overexpressing c-MET protein or expressing constitutively activated c-MET protein. In some embodiments, the c-MET inhibitor is chosen from capmatinib (INC280), JNJ-3887605, AMG 337, LY2801653, MSC2156119J, crizotinib, tivantinib, or golvatinib.

In certain embodiments, a combination described herein comprises a transforming growth factor beta (also known as TGF-β TGFβ, TGFb, or TGF-beta, used interchangeably herein) inhibitor. In some embodiments, the TGF-β inhibitor is chosen from fresolimumab or XOMA 089.

In certain embodiments, a combination described herein comprises an adenosine A2a receptor (A2aR) antagonist (e.g., an inhibitor of A2aR pathway, e.g., an adenosine inhibitor, e.g., an inhibitor of A2aR or CD-73). In some embodiments, the A2aR antagonist is used in combination with a PD-1 inhibitor, and one or more (e.g., two, three, four, five, or all) of a CXCR2 inhibitor, a CSF-1/1R binding agent, LAG-3 inhibitor, a GITR agonist, a c-MET inhibitor, or an IDO inhibitor. In some embodiments, the combination is used to treat a pancreatic cancer, a colorectal cancer, a gastric cancer, or a melanoma (e.g., a refractory melanoma). In some embodiments, the A2aR antagonist is chosen from PBF509 (NIR178) (Palobiofarma/Novartis), CP1444/V81444 (Corvus/Genentech), AZD4635/HTL-1071 (AstraZeneca/Heptares), Vipadenant (Redox/Juno), GBV-2034 (Globavir), AB928 (Arcus Biosciences), Theophylline, Istradefylline (Kyowa Hakko Kogyo), Tozadenant/SYN-115 (Acorda), KW-6356 (Kyowa Hakko Kogyo), ST-4206 (Leadiant Biosciences), or Preladenant/SCH 420814 (Merck/Schering). Without wishing to be bound by theory, it is believed that in some embodiments, inhibition of A2aR leads to upregulation of IL-1b.

In certain embodiments, a combination described herein comprises an inhibitor of indoleamine 2,3-dioxygenase (IDO) and/or tryptophan 2,3-dioxygenase (TDO). In some embodiments, the IDO inhibitor is used in combination with a PD-1 inhibitor, and one or more (e.g., two, three, four, or all) of a TGF-β inhibitor, an A2aR antagonist, a CSF-1/1R binding agent, a c-MET inhibitor, or a GITR agonist. In some embodiments, the combination is used to treat a pancreatic cancer, a colorectal cancer, a gastric cancer, or a melanoma (e.g., a refractory melanoma). In some embodiments, the IDO inhibitor is chosen from (4E)-4-[(3-chloro-4-fluoroanilino)-nitrosomethylidene]-1,2,5-oxadiazol-3-amine (also known as epacadostat or INCB24360), indoximod (NLG8189), (1-methyl-D-tryptophan), α-cyclohexyl-5H-Imidazo[5,1-a]isoindole-5-ethanol (also known as NLG919), indoximod, BMS-986205 (formerly F001287).

In certain embodiments, a combination described herein comprises a Galectin, e.g., Galectin-1 or Galectin-3, inhibitor. In some embodiments, the combination comprises a Galectin-1 inhibitor and a Galectin-3 inhibitor. In some embodiments, the combination comprises a bispecific inhibitor (e.g., a bispecific antibody molecule) targeting both Galectin-1 and Galectin-3. In some embodiments, the Galectin inhibitor is used in combination with one or more therapeutic agents described herein. In some embodiments, the Galectin inhibitor is chosen from an anti-Galectin antibody molecule, GR-MD-02 (Galectin Therapeutics), Galectin-3C (Mandal Med), Anginex, or OTX-008 (OncoEthix, Merck). In some embodiments, a combination described herein comprises an inhibitor of the MAP kinase pathway including ERK inhibitors, MEK inhibitors and RAF inhibitors.

In some embodiments, a combination described herein comprises a MEK inhibitor. In some embodiments, the MEK inhibitor is chosen from Trametinib, selumetinib, AS703026, BIX 02189, BIX 02188, CI-1040, PD0325901, PD98059, U0126, XL-518, G-38963, or G02443714.

In some embodiments, the MEK inhibitor is trametinib. Trametinib is also known as JTP-74057, TMT212, N-(3-{3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl}phenyl)acetamide, or Mekinist (CAS Number 871700-17-3).

In some embodiments, the MEK inhibitor comprises selumetinib which has the chemical name: (5-[(4-bromo-2-chlorophenyl)amino]-4-fluoro-N-(2-hydroxyethoxy)-1-methyl-1H-benzimidazole-6-carboxamide. Selumetinib is also known as AZD6244 or ARRY 142886, e.g., as described in PCT Publication No. WO2003077914.

In some embodiments, the MEK inhibitor comprises AS703026, BIX 02189 or BIX 02188.

In some embodiments, the MEK inhibitor comprises 2-[(2-Chloro-4-iodophenyl)amino]-N-(cyclopropylmethoxy)-3,4-difluoro-benzamide (also known as CI-1040 or PD184352), e.g., as described in PCT Publication No. WO2000035436).

In some embodiments, the MEK inhibitor comprises N-[(2R)-2,3-Dihydroxypropoxy]-3,4-difluoro-2-[(2-fluoro-4-iodophenyl)amino]-benzamide (also known as PD0325901), e.g., as described in PCT Publication No. WO2002006213).

In some embodiments, the MEK inhibitor comprises 2′-amino-3′-methoxyflavone (also known as PD98059) which is available from Biaffin GmbH & Co., KG, Germany.

In some embodiments, the MEK inhibitor comprises 2,3-bis[amino[(2-aminophenyl)thio]methylene]-butanedinitrile (also known as U0126), e.g., as described in U.S. Pat. No. 2,779,780).

In some embodiments, the MEK inhibitor comprises XL-518 (also known as GDC-0973) which has a CAS No. 1029872-29-4 and is available from ACC Corp.

In some embodiments, the MEK inhibitor comprises G-38963.

In some embodiments, the MEK inhibitor comprises G02443714 (also known as AS703206)

Additional examples of MEK inhibitors are disclosed in WO 2013/019906, WO 03/077914, WO 2005/121142, WO 2007/04415, WO 2008/024725 and WO 2009/085983, the contents of which are incorporated herein by reference. Further examples of MEK inhibitors include, but are not limited to, 2,3-Bis[amino[(2-aminophenyl)thio]methylene]-butanedinitrile (also known as U0126 and described in U.S. Pat. No. 2,779,780); (3S,4R,5Z,8S,9S,11E)-14-(Ethylamino)-8,9,16-trihydroxy-3,4-dimethyl-3,4,9,19-tetrahydro-1H-2-benzoxacyclotetradecine-1,7(8H)-dione] (also known as E6201, described in PCT Publication No. WO2003076424); vemurafenib (PLX-4032, CAS 918504-65-1); (R)-3-(2,3-Dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5); pimasertib (AS-703026, CAS 1204531-26-9); 2-(2-Fluoro-4-iodophenylamino)-N-(2-hydroxyethoxy)-1,5-dimethyl-6-oxo-1,6-dihydropyridine-3-carboxamide (AZD 8330); and 3,4-Difluoro-2-[(2-fluoro-4-iodophenyl)amino]-N-(2-hydroxyethoxy)-5-[(3-oxo-[1,2]oxazinan-2-yl)methyl]benzamide (CH 4987655 or Ro 4987655).

In some embodiments, a combination described herein comprises a RAF inhibitor.

RAF inhibitors include, but are not limited to, Vemurafenib (or Zelboraf®, PLX-4032, CAS 918504-65-1), GDC-0879, PLX-4720 (available from Symansis), Dabrafenib (or GSK2118436), LGX 818, CEP-32496, UI-152, RAF 265, Regorafenib (BAY 73-4506), CCT239065, or Sorafenib (or Sorafenib Tosylate, or Nexavar®).

In some embodiments, the RAF inhibitor is Dabrafenib.

In some embodiments, the RAF inhibitor is LXH254.

In some embodiments, a combination described herein comprises an ERK inhibitor.

ERK inhibitors include, but are not limited to, LTT462, ulixertinib (BVD-523), LY3214996, GDC-0994, KO-947 and MK-8353.

In some embodiments, the ERK inhibitor is LTT462. LTT462 is 4-(3-amino-6-((1S,3S,4S)-3-fluoro-4-hydroxy¬cyclohexyl)pyrazin-2-yl)-N—((S)-1-(3-bromo-5-fluorophenyl)-2-(methylamino),ethyl)-2-fluorobenzamide and is the compound of the following structure:

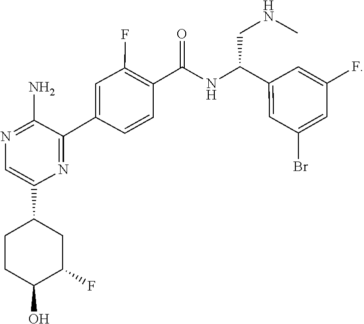 [SMILES: CNC[C@@H](NC(=O)c1ccc(-c2nc([C@H]3CC[C@H](O)[C@@H](F)C3)cnc2N)cc1F)c1cc(F)cc(Br)c1]

The preparation of LTT462 is described in PCT patent application publication WO2015/066188. LTT462 is an inhibitor of extracellular signal-regulated kinases 1 and 2 (ERK 1/2).

In some embodiments, a combination described herein comprises a taxane, a vinca alkaloid, a MEK inhibitor, an ERK inhibitor, or a RAF inhibitor.

In some embodiments, a combination described herein comprises at least two inhibitors selected, independently, from a MEK inhibitor, an ERK inhibitor, and a RAF inhibitor.

In some embodiments, a combination described herein comprises an anti-mitotic drug.

In some embodiments, a combination described herein comprises a taxane.

Taxanes include, but are not limited to, docetaxel, paclitaxel, or cabazitaxel. In some embodiments, the taxane is docetaxel.

In some embodiments, a combination described herein comprises a vinca alkaloid.

Vinca alkaloids include, but are not limited to, vincristine, vinblastine, and leurosine.

In some embodiments, a combination described herein comprises a topoisomerase inhibitor.

Topoisomerase inhibitors include, but are not limited to, topotecan, irinotecan, camptothecin, diflomotecan, lamellarin D, ellipticines, etoposide (VP-16), teniposide, doxorubicin, daunorubicin, mitoxantrone, amsacrine, aurintricarboxylic acid, and HU-331.

In one embodiment, a combination described herein includes an interleukin-1 beta (IL-1p) inhibitor. In some embodiments, the IL-1β inhibitor is chosen from canakinumab, gevokizumab, Anakinra, or Rilonacept.

In certain embodiments, a combination described herein comprises an IL-15/IL-15Ra complex. In some embodiments, the IL-15/IL-15Ra complex is chosen from NIZ985 (Novartis), ATL-803 (Altor) or CYP0150 (Cytune).

In certain embodiments, a combination described herein comprises a mouse double minute 2 homolog (MDM2) inhibitor. The human homolog of MDM2 is also known as HDM2. In some embodiments, an MDM2 inhibitor described herein is also known as a HDM2 inhibitor. In some embodiments, the MDM2 inhibitor is chosen from HDM201 or CGM097.

In an embodiment the MDM2 inhibitor comprises (S)-1-(4-chlorophenyl)-7-isopropoxy-6-methoxy-2-(4-(methyl(((1r,4S)-4-(4-methyl-3-oxopiperazin-1-yl)cyclohexyl)methyl)amino)phenyl)-1,2-dihydroisoquinolin-3(4H)-one (also known as CGM097) or a compound disclosed in PCT Publication No. WO 2011/076786 to treat a disorder, e.g., a disorder described herein). In one embodiment, a therapeutic agent disclosed herein is used in combination with CGM097.

In some embodiments, a combination described herein comprises a hypomethylating agent (HMA). In some embodiments, the HMA is chosen from decitabine or azacitidine.

In some embodiments, a combination described herein comprises a glucocorticoid. In some embodiments, the glucocorticoid is dexamethasone.

In some embodiments, a combination described herein comprises asparaginase.

In certain embodiments, a combination described herein comprises an inhibitor acting on any pro-survival proteins of the Bcl2 family. In certain embodiments, a combination described herein comprises a Bcl-2 inhibitor. In some embodiments, the Bcl-2 inhibitor is venetoclax (also known as ABT-199):

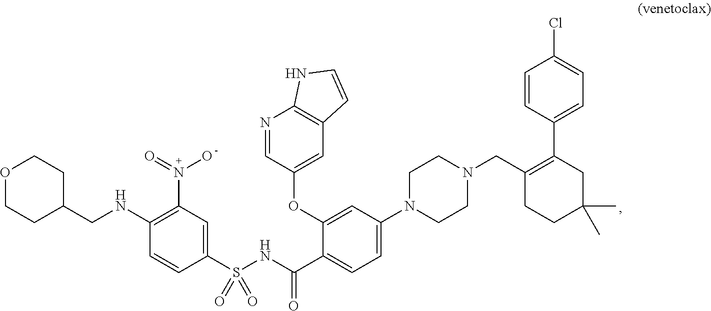

In one embodiment, the Bcl-2 inhibitor is selected from the compounds described in WO 2013/110890 and WO 2015/011400. In some embodiments, the Bcl-2 inhibitor comprises navitoclax (ABT-263), ABT-737, BP1002, SPC2996, APG-1252, obatoclax mesylate (GX15-070MS), PNT2258, Zn-d5, BGB-11417, or oblimersen (G3139). In some embodiments, the Bcl-2 inhibitor is N-(4-hydroxyphenyl)-3-[6-[(3S)-3-(morpholinomethyl)-3,4-dihydro-1H-isoquinoline-2-carbonyl]-1,3-benzodioxol-5-yl]-N-phenyl-5,6,7,8-tetrahydroindolizine-1-carboxamide, compound A1:

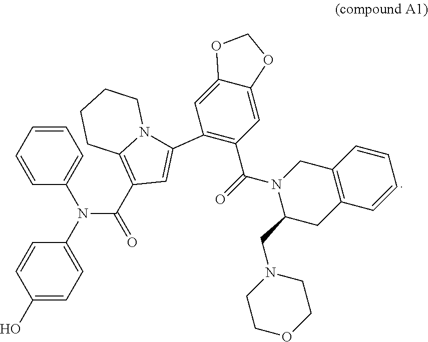 [SMILES: O=C(c1cc(-c2cc3c(cc2C(=O)N2Cc4ccccc4C[C@H]2CN2CCOCC2)OCO3)n2c1CCCC2)N(c1ccccc1)c1ccc(O)cc1]

In some embodiments, the Bcl-2 inhibitor is (S)-5-(5-chloro-2-(3-(morpholinomethyl)-1,2,3,4-tetrahydroisoquinoline-2-carbonyl)phenyl)-N-(5-cyano-1,2-dimethyl-1H-pyrrol-3-yl)-N-(4-hydroxyphenyl)-1,2-dimethyl-1H-pyrrole-3-carboxamide), compound A2:

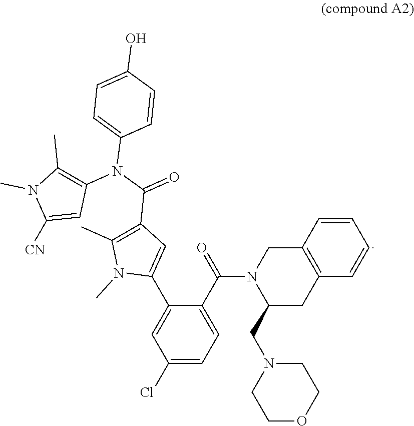 [SMILES: Cc1c(N(C(=O)c2cc(-c3cc(Cl)ccc3C(=O)N3Cc4ccccc4C[C@H]3CN3CCOCC3)n(C)c2C)c2ccc(O)cc2)cc(C#N)n1C]

In one embodiment, the antibody-drug conjugates or combinations disclosed herein are suitable for the treatment of cancer in vivo. For example, the combination can be used to inhibit the growth of cancerous tumors. The combination can also be used in combination with one or more of: a standard of care treatment (e.g., for cancers or infectious disorders), a vaccine (e.g., a therapeutic cancer vaccine), a cell therapy, a hormone therapy (e.g., with anti-estrogens or anti-androgens), a radiation therapy, surgery, or any other therapeutic agent or modality, to treat a disorder herein. For example, to achieve antigen-specific enhancement of immunity, the combination can be administered together with an antigen of interest. A combination disclosed herein can be administered in either order or simultaneously.

### ADDITIONAL EMBODIMENTS

The disclosure provides the following additional embodiments for linker-drug groups, antibody-drug conjugates, conjugate linker groups, and methods of conjugation.

### Linker-Drug Group

In some embodiments, the Linker-Drug group of the invention may be a compound having the structure of Formula (A′), or a pharmaceutically acceptable salt thereof:

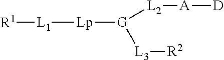

wherein:
   R¹ is a reactive group;
   L₁ is a bridging spacer;
   Lp is a bivalent peptide spacer;
   G-L₂-A is a self-immolative spacer;
   R² is a hydrophilic moiety;
   L₂ is a bond, a methylene, a neopentylene or a C₂-C₃alkenylene;
   A is a bond, —OC(═O)—*,

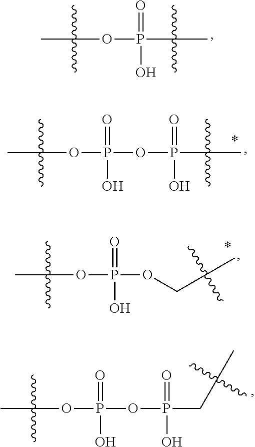 [SMILES: *OP(*)(=O)O ; *OP(=O)(O)OP(*)(=O)O ; *COP(=O)(O)O* ; *CP(=O)(O)OP(=O)(O)O*]

OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D;
L₃ is a spacer moiety; and
D is a Drug moiety that is capable of inhibiting the activity of panRAS (e.g., K-Ras (including splice variants KRAS4A and KRAS4B), H-Ras and N-Ras) when, e.g., released from the Antibody Drug Conjugates or immunoconjugates disclosed herein.

Certain aspects and examples of the Linker-Drug group of the invention are provided in the following listing of enumerated embodiments. It will be recognized that features specified in each embodiment may be combined with other specified features to provide further embodiments of the present invention.

Embodiment 1. The compound of Formula (A′), or pharmaceutically acceptable salt thereof, wherein:
R¹ is a reactive group;
L₁ is a bridging spacer;
Lp is a bivalent peptide spacer comprising two to four amino acid residues;
G-L₂-A is a self-immolative spacer;
R² is a hydrophilic moiety;
L₂ is a bond, a methylene_, a neopentylene or a C₂-C₃alkenylene;
A is a bond, —OC(═O)—*,

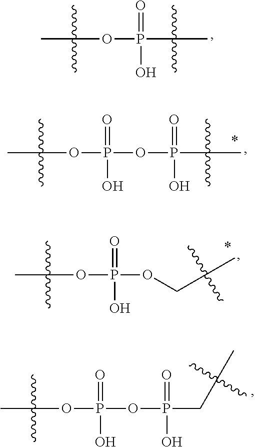 [SMILES: *OP(*)(=O)O ; *OP(=O)(O)OP(*)(=O)O ; *COP(=O)(O)O* ; *CP(=O)(O)OP(=O)(O)O*]

OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D;
L₃ is a spacer moiety; and
D is a Drug moiety as defined herein, e.g., a panRAS inhibitor.

Embodiment 2. The compound of Formula (A′), or pharmaceutically acceptable salt thereof, wherein:
R¹ is a reactive group;
L₁ is a bridging spacer;
Lp is a bivalent peptide spacer comprising two to four amino acid residues;
the

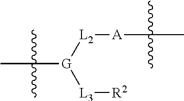

group is selected from:

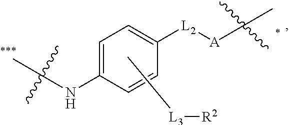

wherein the * of

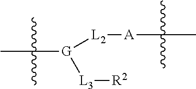

indicates the point of attachment to D (e.g., to an N or a O of the Drug moiety), the *** of

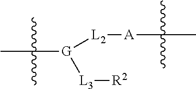

indicates the point of attachment to Lp;
R² is a hydrophilic moiety;
L₂ is a bond, a methylene, a neopentylene or a C₂-C₃alkenylene;
A is a bond, —OC(═O)—*,

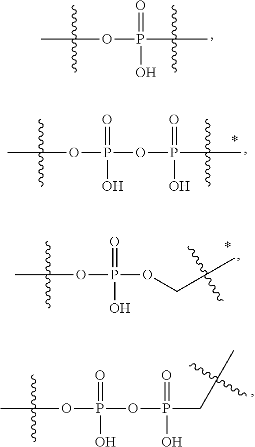 [SMILES: *OP(*)(=O)O ; *OP(=O)(O)OP(*)(=O)O ; *COP(=O)(O)O* ; *CP(=O)(O)OP(=O)(O)O*]

OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆alkyl, and C₃-C₈ cycloalkyl and the * of A indicates the point of attachment to D;
L₃ is a spacer moiety; and
D is a Drug moiety as defined herein, e.g., a panRAS inhibitor.

Embodiment 3. The compound of Formula (A′), or pharmaceutically acceptable salt thereof, having the structure of Formula (B′):

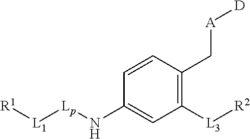

wherein:
   R¹ is a reactive group;
   L₁ is a bridging spacer;
   Lp is a bivalent peptide spacer comprising two to four amino acid residues;
   R² is a hydrophilic moiety;
   A is a bond, —OC(═O)—*,

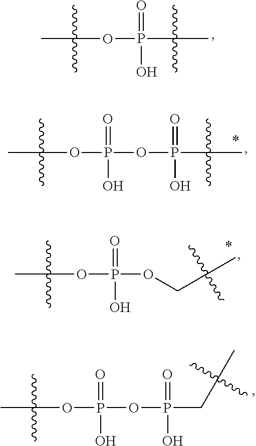 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C1-C₆ alkyl, and C₃-C₈ cycloalkyl and the * of A indicates the point of attachment to D;
L₃ is a spacer moiety; and
D is a Drug moiety as defined herein and comprising an N, wherein D is connected to A via a direct bond from A to the N of the Drug moiety.

Embodiment 4. The compound of Formula (A′) or of any one of Embodiments 1 to 3, or pharmaceutically acceptable salt thereof, wherein:
R¹ is

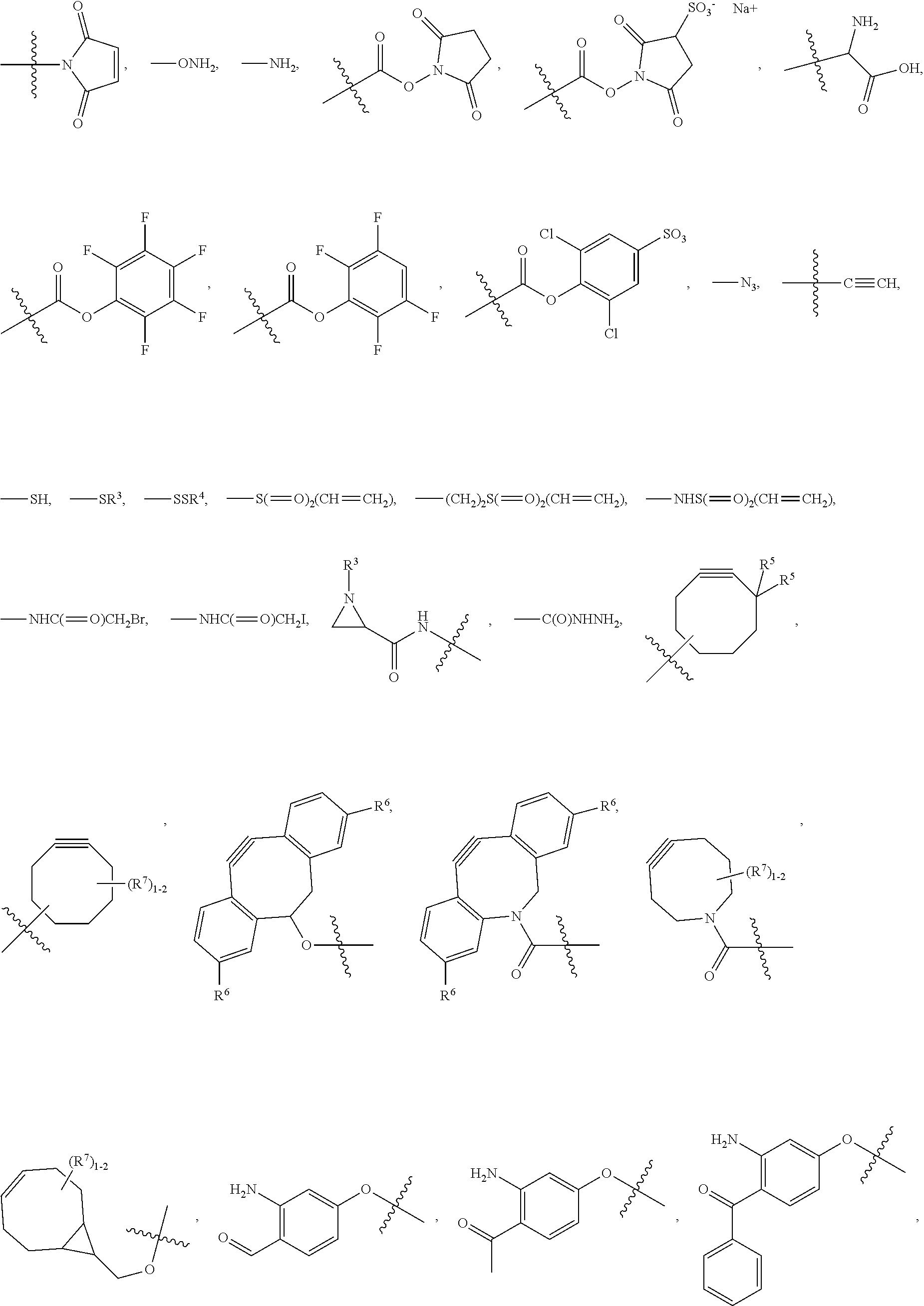

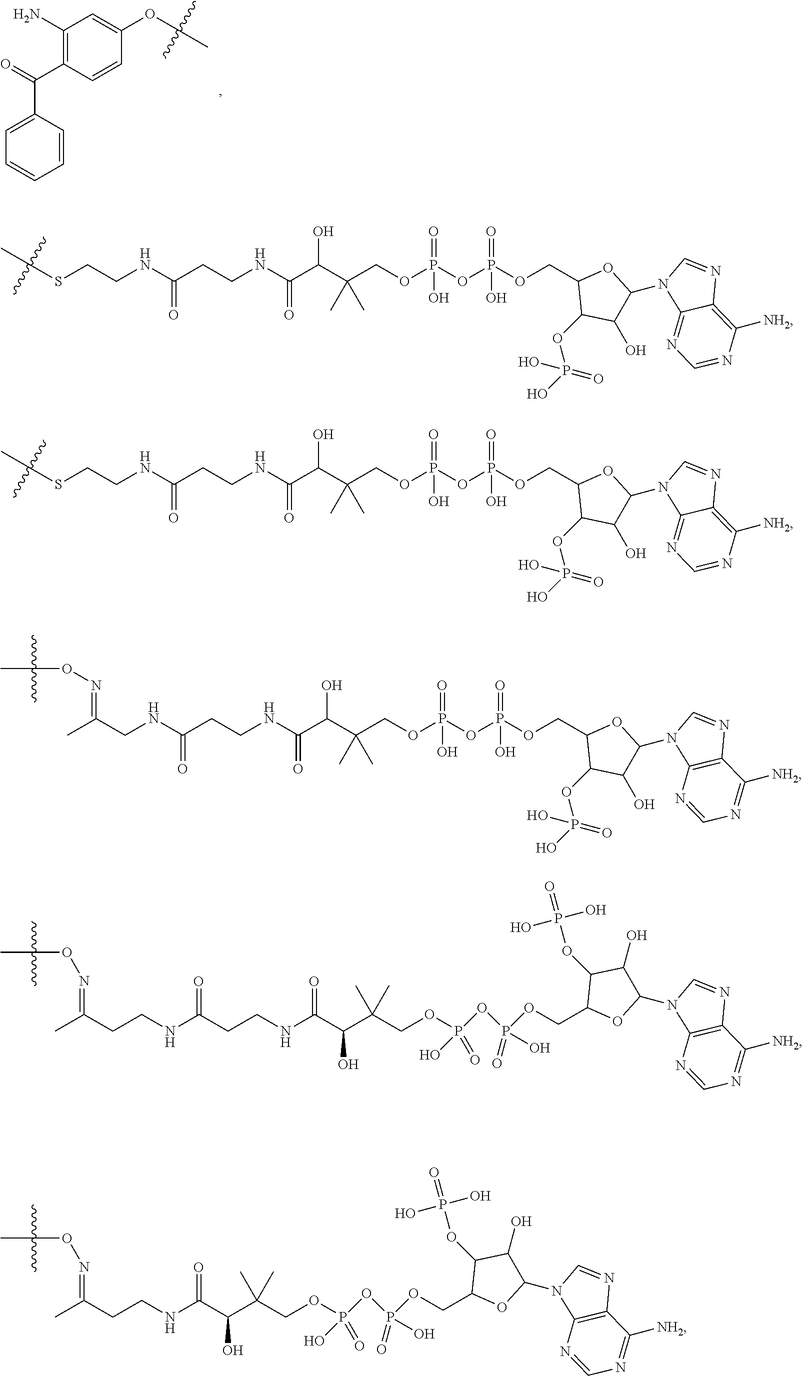

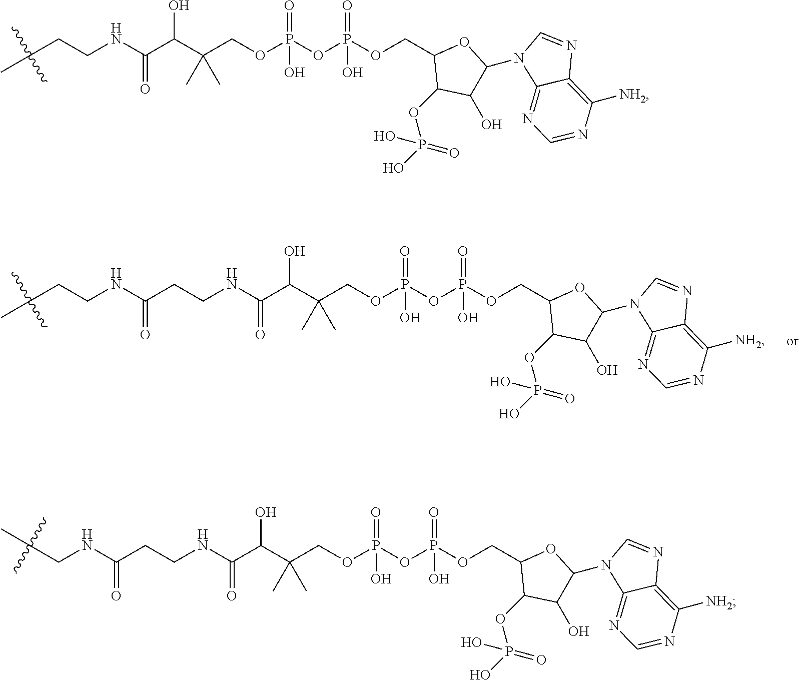 [SMILES: *CCNC(=O)C(O)C(C)(C)COP(=O)(O)OP(=O)(O)OCC1OC(n2cnc3c(N)ncnc32)C(O)C1OP(=O)(O)O ; *CCNC(=O)CCNC(=O)C(O)C(C)(C)COP(=O)(O)OP(=O)(O)OCC1OC(n2cnc3c(N)ncnc32)C(O)C1OP(=O)(O)O ; *CNC(=O)CCNC(=O)C(O)C(C)(C)COP(=O)(O)OP(=O)(O)OCC1OC(n2cnc3c(N)ncnc32)C(O)C1OP(=O)(O)O]

L₁ is *—C(═O)(CH₂)ₘO(CH₂)ₘ—**;
   *—C(═O)((CH₂)ₘO)ₜ(CH₂)ₙ—**;
   *—C(═O)(CH₂)ₘ—**;
   *—C(═O)NH((CH₂)ₘO)ₜ(CH₂)ₙ—**;
   *—C(═O)O(CH₂)ₘSSC(R³)₂(CH₂)ₘC(═O)NR³(CH₂)ₘNR³C(═O)(CH₂)ₘ—**;
   *—C(═O)O(CH₂)ₘC(═O)NH(CH₂)ₘ—**;
   *—C(═O)(CH₂)ₘNH(CH₂)ₘ—**; *—C(═O)(CH₂)ₘNH(CH₂)ₙC(═O)—**;
   *—C(═O)(CH₂)ₘX₁(CH₂)ₘ—**; *—C(═O)((CH₂)ₘO)ₜ(CH₂)ₙX₁(CH₂)ₙ—**;
   *—C(═O)(CH₂)ₘNHC(═O)(CH₂)ₙ—**;
   *—C(═O)((CH₂)ₘO)ₜ(CH₂)ₙNHC(═O)(CH₂)ₙ—**;
   *—C(═O)(CH₂)ₘNHC(═O)(CH₂)ₙX₁(CH₂)ₙ—**;
   *—C(═O)((CH₂)ₘO)ₜ(CH₂)ₙNHC(═O)(CH₂)ₙX₁(CH₂)ₙ—**;
   *—C(═O)((CH₂)ₘO)ₜ(CH₂)ₙC(═O)NH(CH₂)ₘ—**;
   *—C(═O)(CH₂)ₘC(R³)₂—**; or
   *—C(═O)(CH₂)ₘC(═O)N H(CH₂)ₘ—**,
where the * of L₁ indicates the point of attachment to Lp, and the ** of L₁ indicates the point of attachment to R¹;
R² is a hydrophilic moiety selected from polyethylene glycol, polyalkylene glycol, a sugar, an oligosaccharide, a polypeptide or C₂-C₆alkyl substituted with 1 to 3

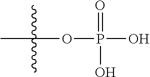 [SMILES: *OP(=O)(O)O]

groups;
each R³ is independently selected from H and C₁-C₆alkyl;
R⁴ is 2-pyridyl or 4-pyridyl;
each R⁵ is independently selected from H, C₁-C₆alkyl, F, Cl, and —OH;
each R⁶ is independently selected from H, C₁-C₆alkyl, F, Cl, —NH₂, —OCH₃, —OCH₂CH₃, —N(CH₃)₂, —CN, —NO₂ and —OH;
each R⁷ is independently selected from H, C₁₋₆-alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
X₁ is

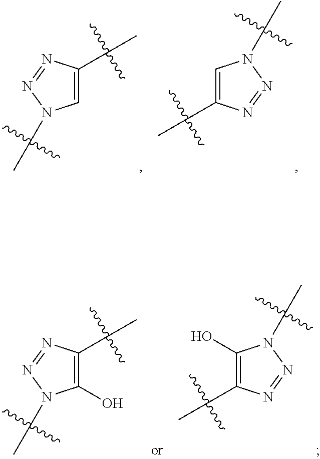 [SMILES: *c1cn(*)nn1 ; *c1cn(*)nn1 ; *c1nnn(*)c1O ; *c1nnn(*)c1O]

each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer comprising an amino acid residue selected from glycine, valine, citrulline, lysine, isoleucine, phenylalanine, methionine, asparagine, proline, alanine, leucine, tryptophan, and tyrosine;
A is a bond, —OC(═O)—*,

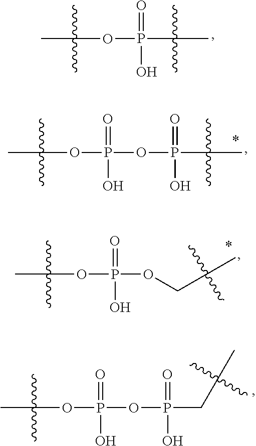 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D;
L₃ is a spacer moiety having the structure

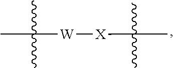

where
   W is —CH₂O—**, —CH₂N(R^{b})C(═O)O—**, —NHC(═O)C(R^{b})₂NHC(═O)O—**, —NHC(═O)C(R^{b})₂NH—**, —NHC(═O)C(R^{b})₂NHC(═O)—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²; and
   the * of L₃ indicates the point of attachment to R²;
and
D is a Drug moiety as defined herein and comprising an N or an O, wherein D is connected to A via a direct bond from A to the N or the O of the Drug moiety.

Embodiment 5. The compound of Formula (A′) or of any one of Embodiments 1 to 4, or pharmaceutically acceptable salt thereof, wherein:
R¹ is

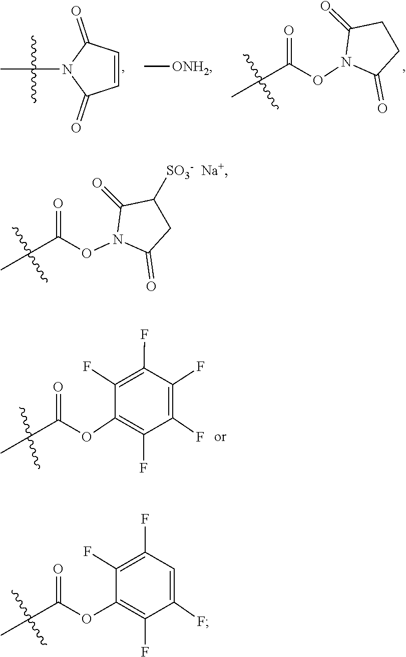

L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp, and the ** of L₁ indicates the point of attachment to R¹;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

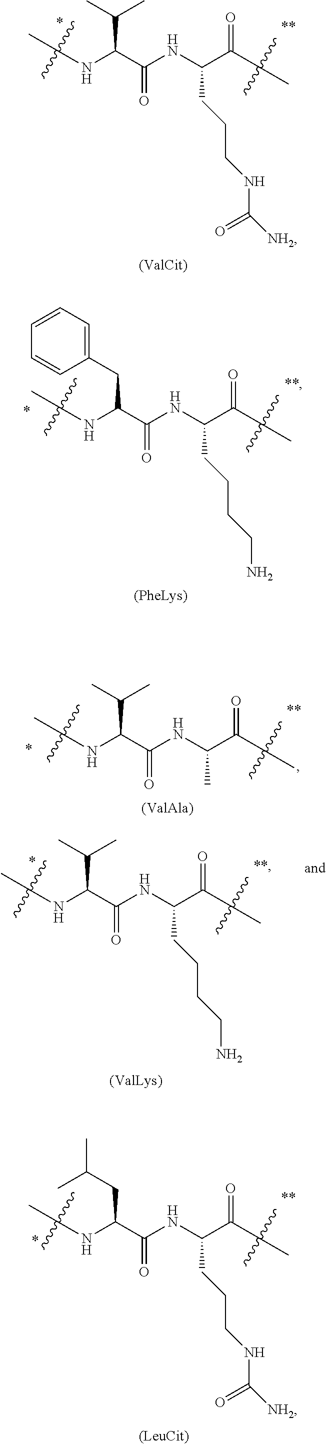

where the * of Lp indicates the attachment point to L₁ and the ** of Lp indicates the attachment point to the —NH— group of G;
L₃ is a spacer moiety having the structure

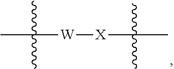

where
   W is —CH₂O—**, —CH₂N(R^{b})C(═O)O—**, —NHC(═O)CH₂NHC(═O)O—**, —NHC(═O)CH₂NH—**, —NHC(═O)CH₂NHC(═O)—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈acycloalkyl and wherein the ** of W indicates the point of attachment to X;
   X is a bond, triazolyl or ***—CH2-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide, C₂-C₆alkyl substituted with 1 to 3

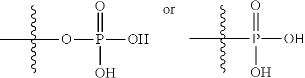 [SMILES: *OP(=O)(O)O ; *P(=O)(O)O]

A is a bond, —OC(═O)—*,

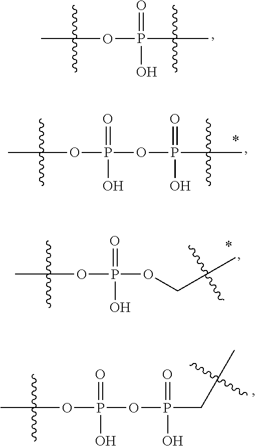 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D; and
D is a Drug moiety as defined herein and comprising an N or an O, wherein D is connected to A via a direct bond from A to the N or the O of the Drug moiety.

Embodiment 6. The compound of Formula (A′) or of any one of Embodiments 1 to 5, or pharmaceutically acceptable salt thereof, wherein:
R¹ is

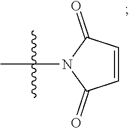 [SMILES: *N1C(=O)C=CC1=O]

L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp, and the ** of L₁ indicates the point of attachment to R¹;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

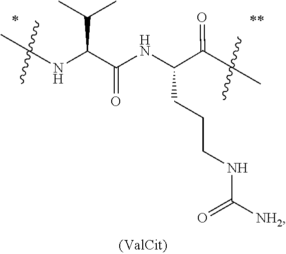 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of G;
L₃ is a spacer moiety having the structure

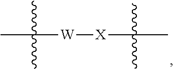

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈acycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide, C₂-C₆alkyl substituted with 1 to 3

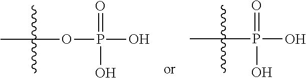 [SMILES: *OP(=O)(O)O ; *P(=O)(O)O]

groups;
A is a bond, —OC(═O)—*,

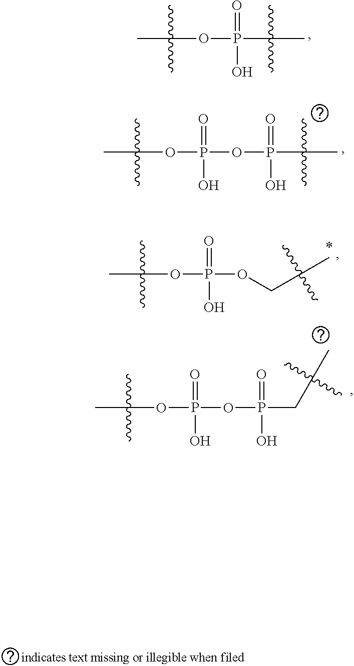 [SMILES: *OP(*)(=O)O ; *OP(=O)(O)OP(*)(=O)O ; "," ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D;
and
D is a Drug moiety as defined herein and comprising an N or an O, wherein D is connected to A via a direct bond from A to the N or the O of the Drug moiety.

Embodiment 7. The compound of Formula (A′) or of any one of Embodiments 1 to 6, or pharmaceutically acceptable salt thereof, wherein:

R¹ is

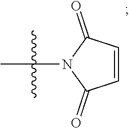 [SMILES: *N1C(=O)C=CC1=O]

L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp and the ** of L₁ indicates the point of attachment to R¹;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

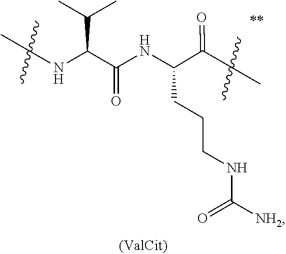 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of G;
L₃ is a spacer moiety having the structure

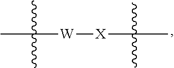

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, or —NHC(═O)NH—**, wherein each R^{b} is independently selected from H, C₁-C₆alkyl or C₃-C₈acycloalkyl and wherein the ** of W indicates the point of attachment to X;
   X is a bond, triazolyl or ***—CH2-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide, C₂-C₆alkyl substituted with 1 to 3

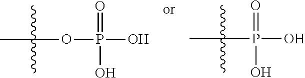 [SMILES: *OP(=O)(O)O ; *P(=O)(O)O]

A is a bond or —OC(═O)*, in which * indicates the attachment point to D;
and
D is a Drug moiety as defined herein and comprising an N or an O, wherein D is connected to A via a direct bond from A to the N or the O of the Drug moiety.

Embodiment 8. The compound of Formula (A′) or of any one of Embodiments 1 to 7, or pharmaceutically acceptable salt thereof, wherein:
R¹ is

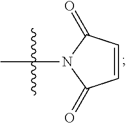 [SMILES: *N1C(=O)C=CC1=O]

L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp and the ** of L₁ indicates the point of attachment to R¹:
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

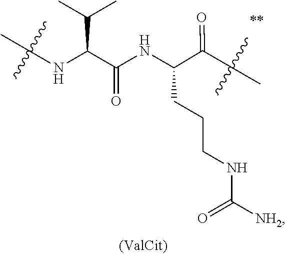 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of G;
L₃ is a spacer moiety having the structure

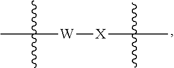

where
   W is —CH₂O—**, —CH₂N(R^{b})C(═O)O—**, —NHC(═O)CH₂NHC(═O)O—**, —CH₂N(X—R²)C(═O)O—**, or —C(═O)N(X—R²)—**, wherein each R^{b} is independently selected from H, C₁-C₆alkyl or C₃-C₈acycloalkyl and wherein the ** of W indicates the point of attachment to X;
   X is ***—CH₂-triazolyl-*, wherein the *** of X indicates the point of attachment to W and the * of X indicates the point of attachment to R²; and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

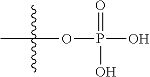 [SMILES: *OP(=O)(O)O]

groups;
A is a bond or —OC(═O)* in which * indicates the attachment point to D;
and
D is a Drug moiety as defined herein and comprising an N or an O, wherein D is connected to A via a direct bond from A to the N or the O of the Drug moiety.

Embodiment 9. The compound of Formula (A′) or of any one of Embodiments 1 to 8, or pharmaceutically acceptable salt thereof, wherein R¹ is a reactive group selected from Table 8.

Embodiment 10. The compound of Formula (A′) or of any one of Embodiments 1 to 9, or pharmaceutically acceptable salt thereof, wherein:
R₁ is

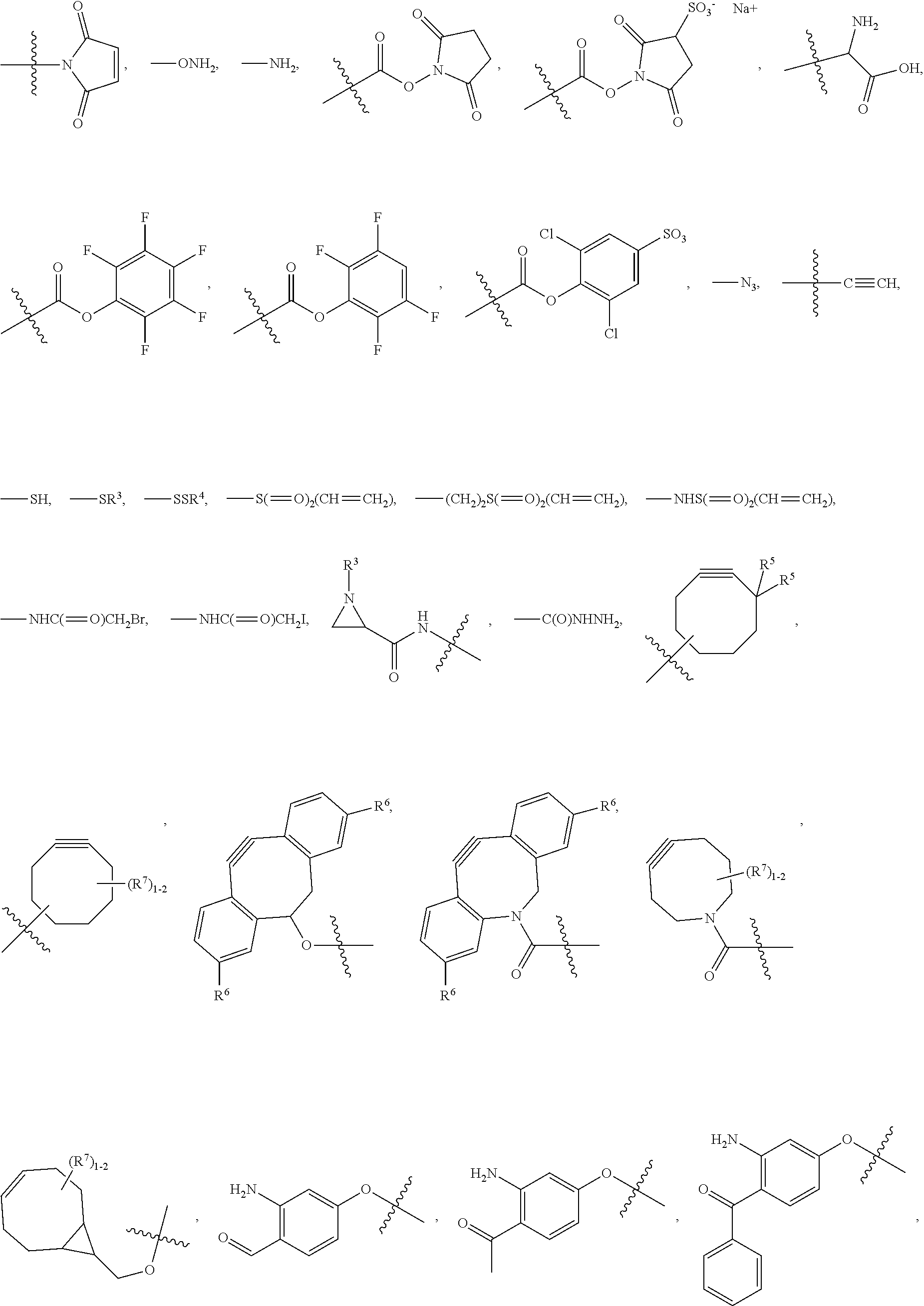

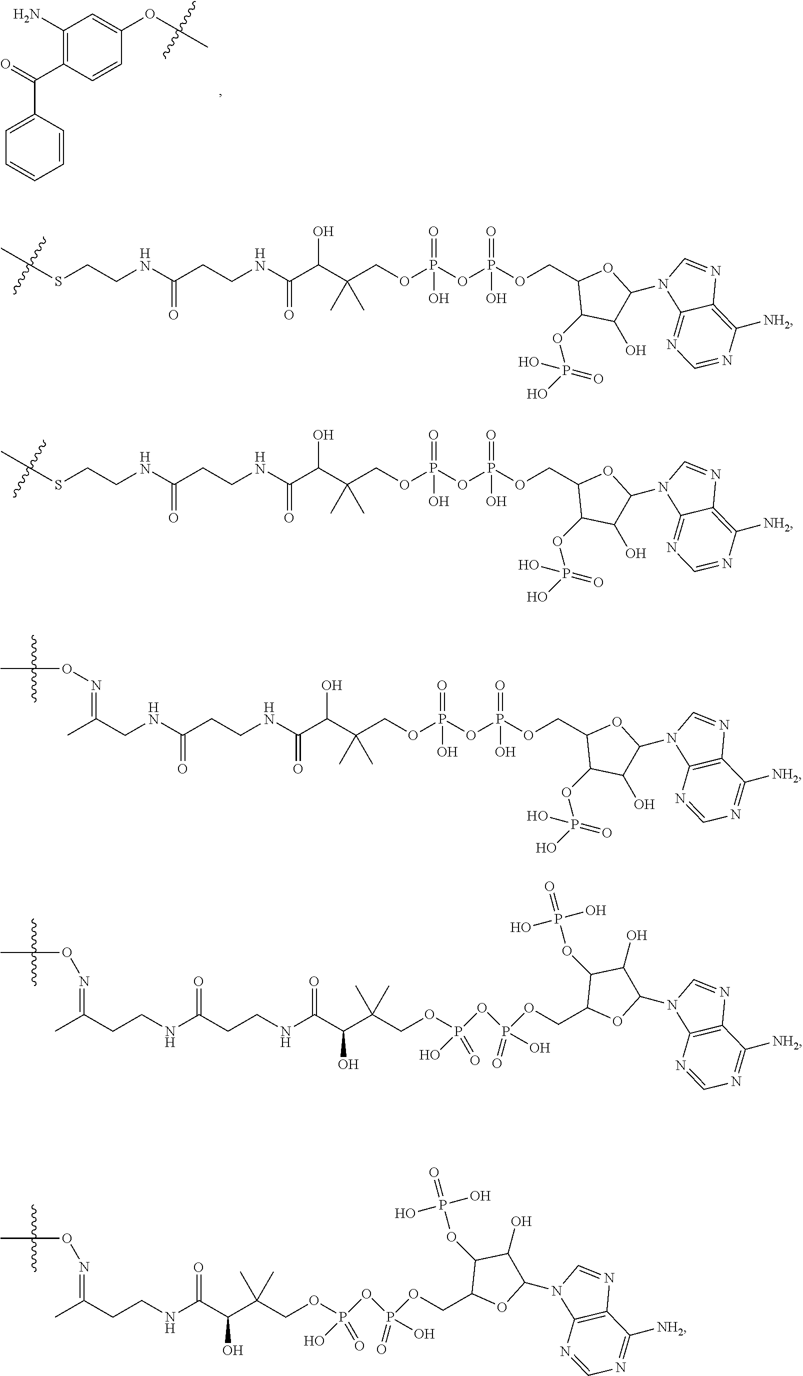

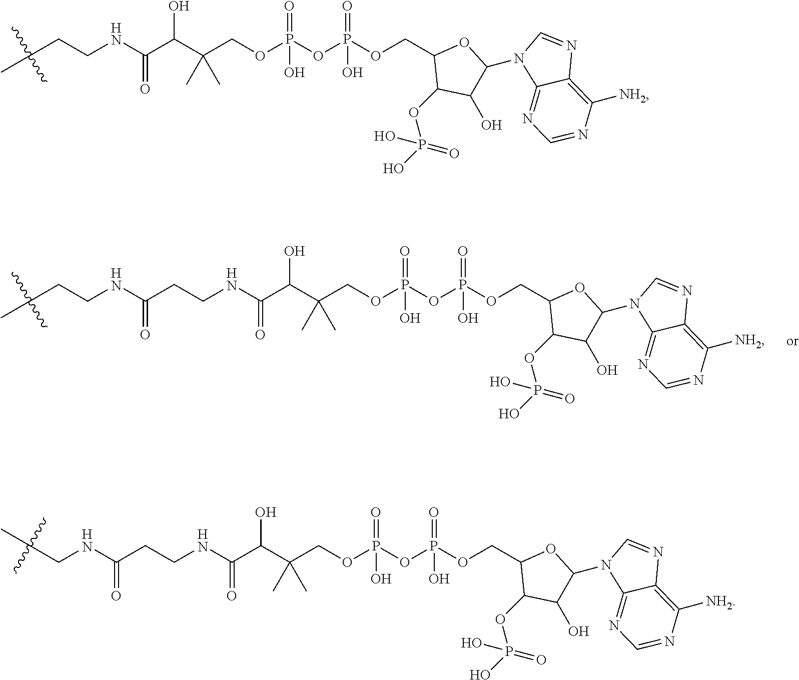 [SMILES: *CCNC(=O)C(O)C(C)(C)COP(=O)(O)OP(=O)(O)OCC1OC(n2cnc3c(N)ncnc32)C(O)C1OP(=O)(O)O ; *CCNC(=O)CCNC(=O)C(O)C(C)(C)COP(=O)(O)OP(=O)(O)OCC1OC(n2cnc3c(N)ncnc32)C(O)C1OP(=O)(O)O ; *CNC(=O)CCNC(=O)C(O)C(C)(C)COP(=O)(O)OP(=O)(O)OCC1OC(n2cnc3c(N)ncnc32)C(O)C1OP(=O)(O)O]

Embodiment 11. The compound of Formula (A′) or of any one of Embodiments 1 to 9, or pharmaceutically acceptable salt thereof, wherein:
R¹ is

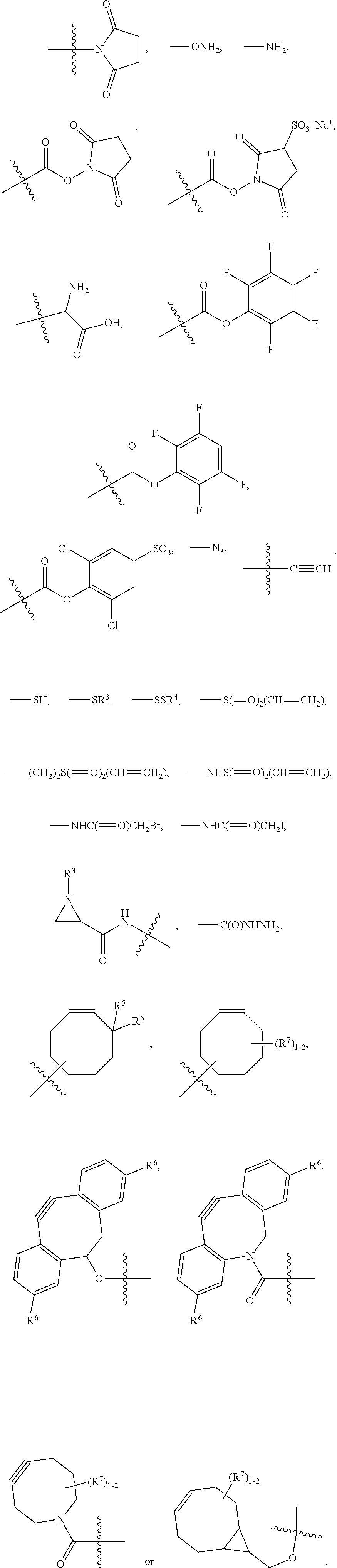

Embodiment 12. The compound of Formula (A′) or of any one of Embodiments 1 to 9, or pharmaceutically acceptable salt thereof, wherein:
R¹ is

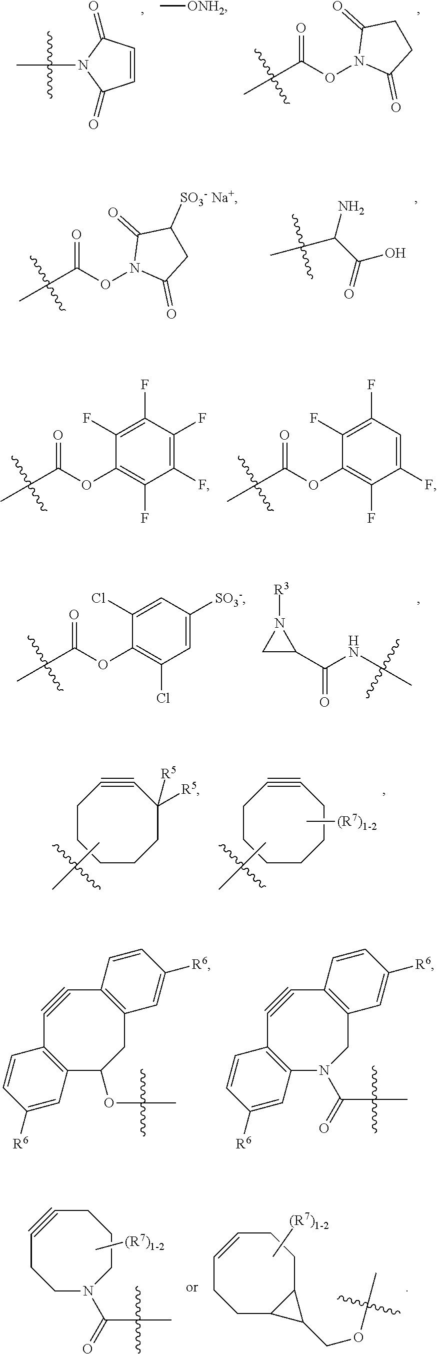

Embodiment 13. The compound of Formula (A′) or of any one of Embodiments 1 to 9, or pharmaceutically acceptable salt thereof, wherein:
R¹ is

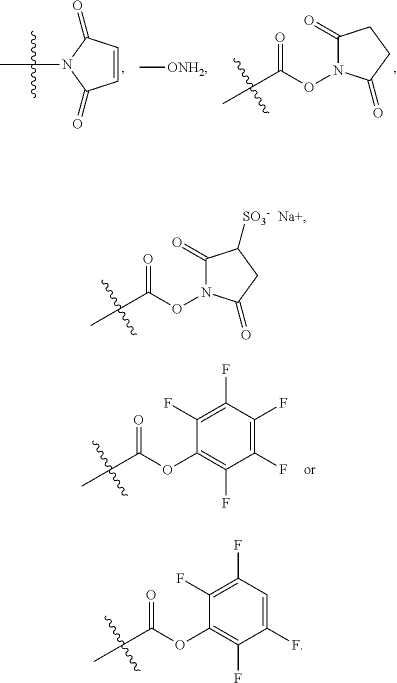

Embodiment 14. The compound of Formula (A′) or of any one of Embodiments 1 to 9, or pharmaceutically acceptable salt thereof, wherein R¹ is

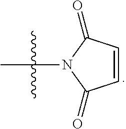 [SMILES: *N1C(=O)C=CC1=O ; "."]

Embodiment 15. The compound of Formula (A′) or of any one of Embodiments 1 to 9, or pharmaceutically acceptable salt thereof, wherein R¹ is —ONH₂.

Embodiment 16. The compound of Formula (A′) or of any one of Embodiments 1 to 9, or pharmaceutically acceptable salt thereof, wherein: R¹ is

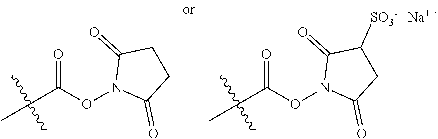

Embodiment 17. The compound of Formula (A′) or of any one of Embodiments 1 to 9, or pharmaceutically acceptable salt thereof, wherein:
R¹ is or

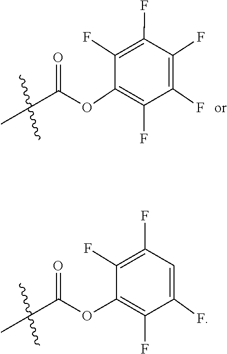 [SMILES: *C(=O)Oc1c(F)c(F)c(F)c(F)c1F ; *C(=O)Oc1c(F)c(F)cc(F)c1F]

Embodiment 18. The compound of Formula (A′) or of any one of Embodiments 1 to 9, or pharmaceutically acceptable salt thereof, having the structure:

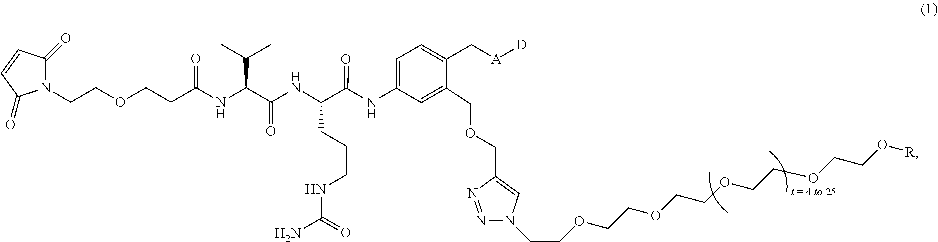

where
R is H, —CH₃ or —CH₂CH₂C(═O)OH.

Embodiment 19. The compound of Formula (A′) or of any one of Embodiments 1 to 9, or pharmaceutically acceptable salt thereof, having the structure:

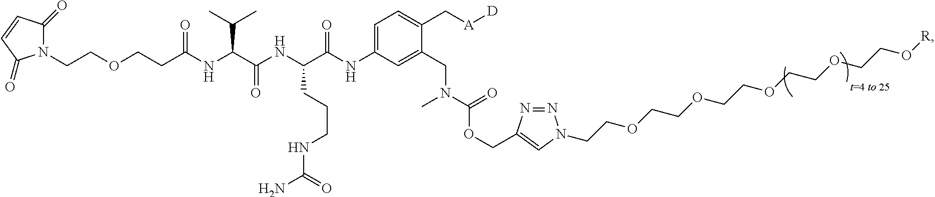

where
R is H, —CH₃ or —CH₂CH₂C(═O)OH.

Embodiment 20. The compound of Formula (A′) or of any one of Embodiments 1 to 9, or pharmaceutically acceptable salt thereof, having the structure:

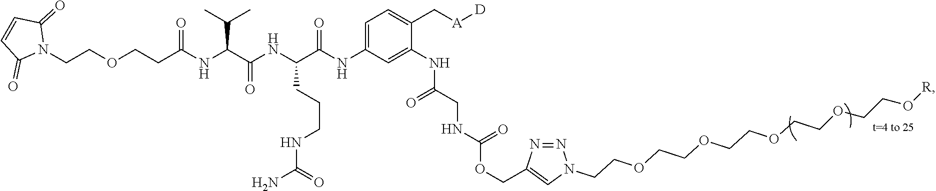

where
R is H, —CH₃ or —CH₂CH₂C(═O)OH.

Embodiment 21. The compound of Formula (A′) or of any one of Embodiments 1 to 9, or pharmaceutically acceptable salt thereof, having the structure:

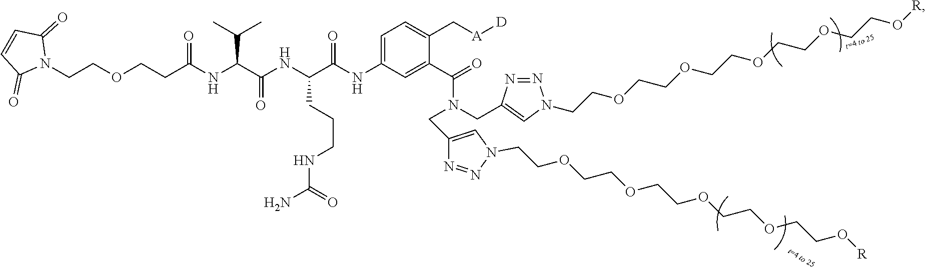

where
each R is independently selected from H, —CH₃ or —CH₂CH₂C(═O)OH.

Embodiment 22. The compound of Formula (A′) or of any one of Embodiments 1 to 9, or pharmaceutically acceptable salt thereof, having the structure:

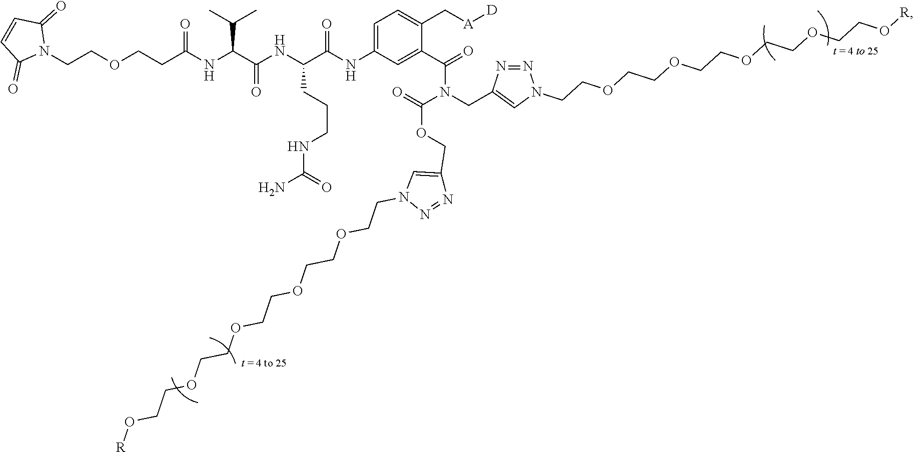

where
each R is independently selected from H, —CH₃ or —CH₂CH₂C(═O)OH.

Embodiment 23. The compound of Formula (A′) or of any one of Embodiments 1 to 9 or pharmaceutically acceptable salt thereof, having the structure:

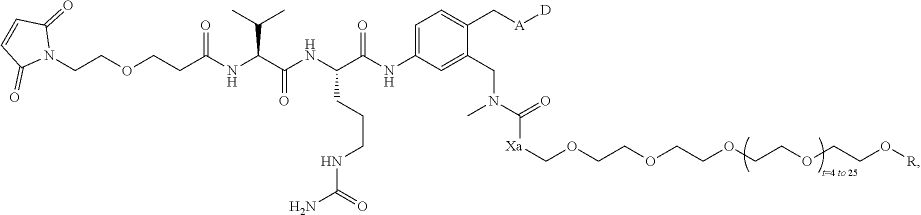

where
Xa is —CH₂—, —OCH₂—, —NHCH₂— or —NRCH₂— and each R independently is H, —CH₃ or —CH₂CH₂C(═O)OH.

Embodiment 24. The compound of Formula (A′) or of any one of Embodiments 1 to 9, or pharmaceutically acceptable salt thereof, having the structure:

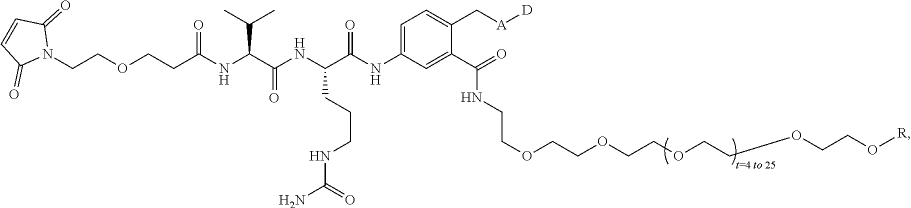

where
R is H, —CH₃ or —CH₂CH₂C(═O)OH.

Embodiment 25. The compound of Formula (A′) or of any one of Embodiments 1 to 9, or pharmaceutically acceptable salt thereof, having the structure:

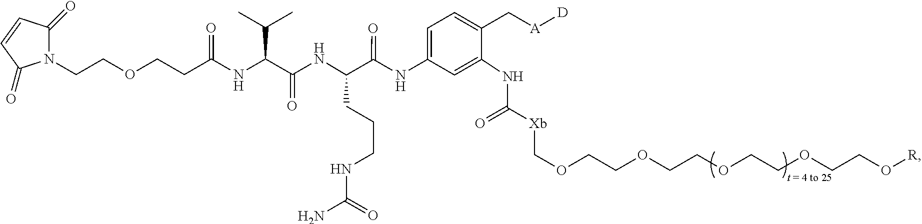

where
Xb is —CH₂—, —OCH₂—, —NHCH₂— or —NRCH₂— and each R independently is H, —CH₃ or —CH₂CH₂C(═O)OH.

Embodiment 26. The compound of Formula (A′) or of any one of Embodiments 1 to 9, or pharmaceutically acceptable salt thereof, having the structure:

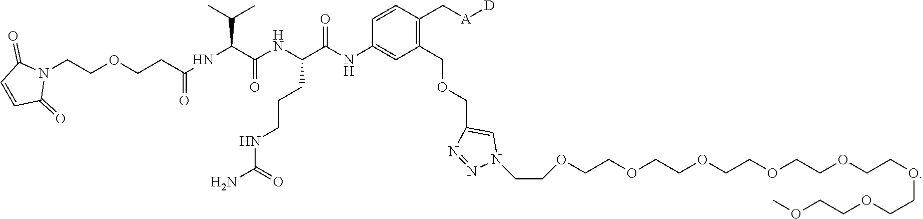

Embodiment 27. The compound of Formula (A′) or of any one of Embodiments 1 to 9, or pharmaceutically acceptable salt thereof, having the structure:

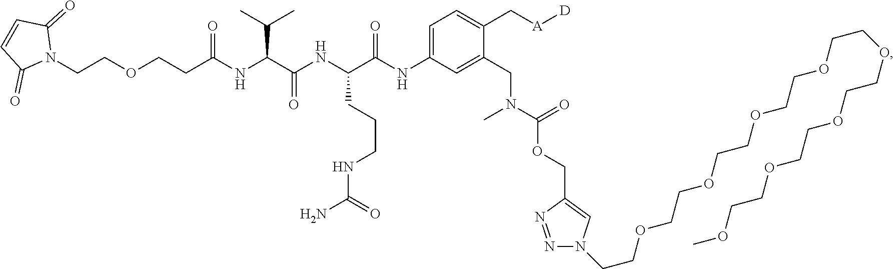

Embodiment 28. The compound of Formula (A′) or of any one of Embodiments 1 to 9, or pharmaceutically acceptable salt thereof, having the structure:

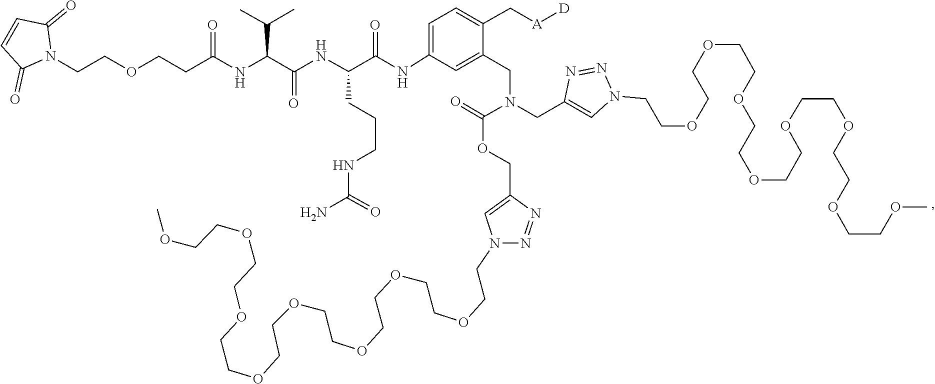

Embodiment 29. The compound of Formula (A′) or of any one of Embodiments 1 to 9, or pharmaceutically acceptable salt thereof, having the structure:

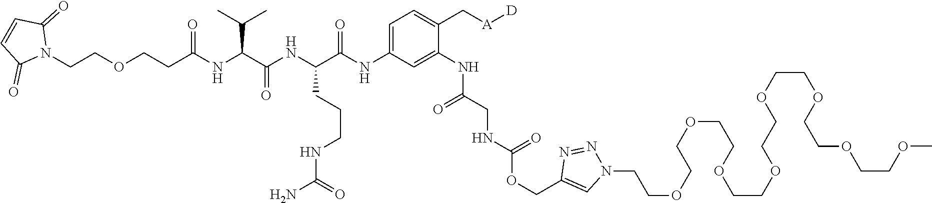

Embodiment 30. The compound of Formula (A′) or of any one of Embodiments 1 to 9, or pharmaceutically acceptable salt thereof, having the structure:

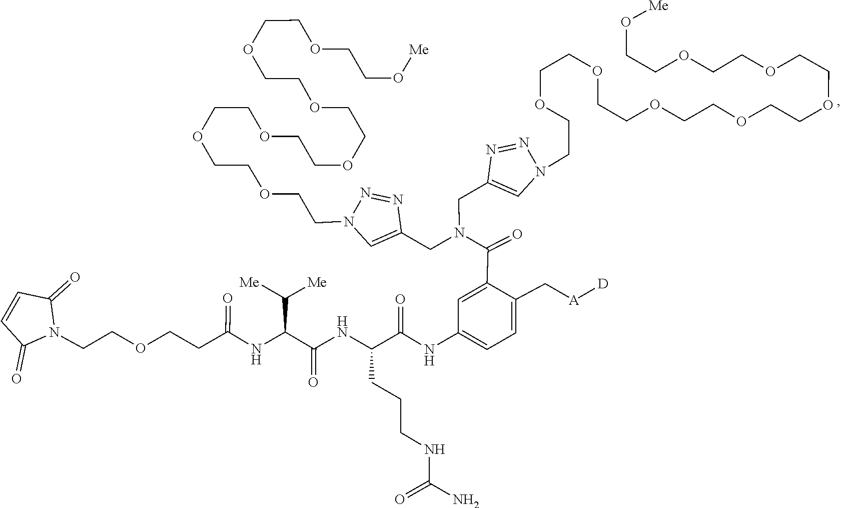

Embodiment 31. The compound of Formula (A′) or of any one of Embodiments 1 to 9, or pharmaceutically acceptable salt thereof, having the structure:

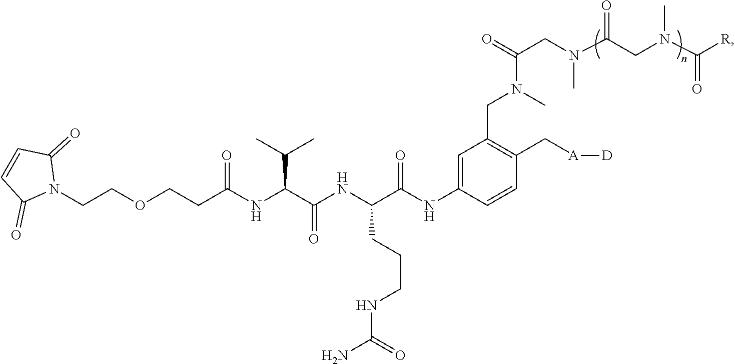

where n is an integer between 2 and 24.

Embodiment 32. The compound of Formula (A′) or of any one of Embodiments 1 to 9, or pharmaceutically acceptable salt thereof, having the structure of a compound in Table B.

Embodiment 33. A conjugate linker of the Linker-Drug group of Formula (A′) having the structure of Formula (C′),

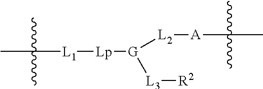

wherein
   L₁ is a bridging spacer;
   Lp is a bivalent peptide spacer;
   G-L₂-A is a self-immolative spacer;
   R² is a hydrophilic moiety;
   L₂ is a bond, a methylene, a neopentylene or a C₂-C₃alkenylene;
   A is a bond, —OC(═O)—*,

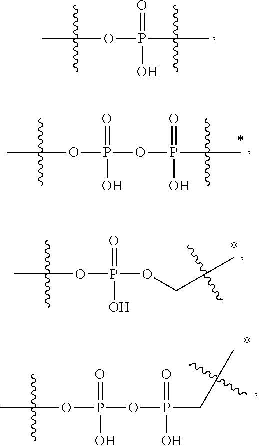 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D,
and
L₃ is a spacer moiety.

Embodiment 34. The conjugate linker of Embodiment 33, wherein:
L₁ is a bridging spacer;
Lp is a bivalent peptide spacer comprising two to four amino acid residues;
G-L₂-A is a self-immolative spacer;
R² is a hydrophilic moiety;
L₂ is a bond, a methylene, a neopentylene or a C₂-C₃alkenylene;
A is a bond, —OC(═O)—*,

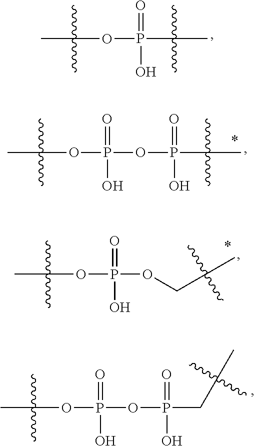 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D,
and
L₃ is a spacer moiety.

Embodiment 35. The conjugate linker of Embodiment 33 or 34, wherein:
L₁ is a bridging spacer;
Lp is a bivalent peptide spacer comprising two to four amino acid residues;
the

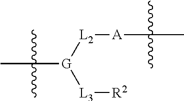

group is selected from:

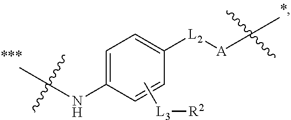

wherein the * of

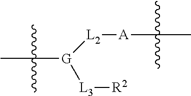

indicates the point of attachment to D (e.g., to an N or a O of the Drug moiety), the *** of

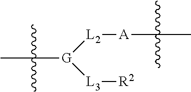

indicates the point of attachment to Lp;
R² is a hydrophilic moiety;
L₂ is a bond, a methylene, a neopentylene or a C₂-C₃alkenylene;
A is a bond, —OC(═O)—*,

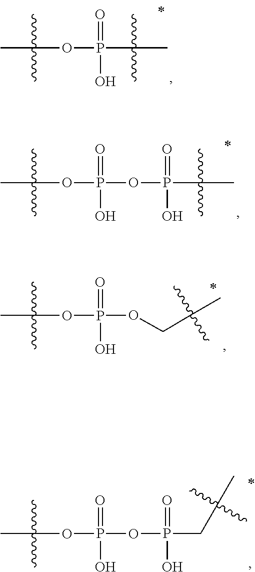 [SMILES: *OP(*)(=O)O ; *OP(=O)(O)OP(*)(=O)O ; *COP(=O)(O)O* ; *CP(=O)(O)OP(=O)(O)O*]

OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D,
and
L₃ is a spacer moiety.

Embodiment 36. The conjugate linker of any one of Embodiments 33 to 35, wherein:
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; *—C(═O)NH((CH₂)ₘO)ₜ(CH₂)ₙ—**; *—C(═O)O(CH₂)ₘSSC(R³)₂(CH₂)ₘC(═O)NR³(CH₂)ₘNR³C(═O)(CH₂)ₘ—**; *—C(═O)O(CH₂)ₘC(═O)NH(CH₂)ₘ—**; *—C(═O)(CH₂)ₘNH(CH₂)ₘ—**; *—C(═O)(CH₂)ₘNH(CH₂)ₙC(═O)—**; *—C(═O)(CH₂)ₘX₁(CH₂)ₘ—**; *—C(═O)((CH₂)ₘO)ₜ(CH₂)ₙX₁(CH₂)ₙ—**; *—C(═O)(CH₂)ₘNHC(═O)(CH₂)ₙ—**; *—C(═O)((CH₂)ₘO)ₜ(CH₂)ₙNHC(═O)(CH₂)ₙ—**; *—C(═O)(CH₂)ₘNHC(═O)(CH₂)ₙX₁(CH₂)ₙ—**; *—C(═O)((CH₂)ₘO)ₜ(CH₂)ₙNHC(═O)(CH₂)ₙX₁(CH₂)ₙ—**; *—C(═O)((CH₂)ₘO)ₜ(CH₂)ₙC(═O)NH(CH₂)ₘ—**; *—C(═O)(CH₂)ₘC(R³)₂—** or *—C(═O)(CH₂)ₘC(═O)NH(CH₂)ₘ—**, where the * of L₁ indicates the point of attachment to Lp;
R² is a hydrophilic moiety selected from polyethylene glycol, polyalkylene glycol, a sugar, an oligosaccharide, a polypeptide or C₂-C₆alkyl substituted with 1 to 3

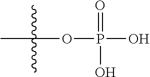 [SMILES: *OP(=O)(O)O]

groups;
each R³ is independently selected from H and C₁-C₆alkyl;
X₁ is

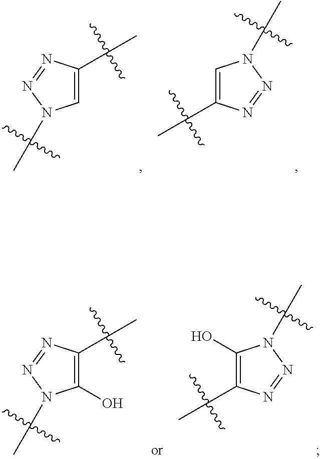 [SMILES: *c1cn(*)nn1 ; *c1cn(*)nn1 ; *c1nnn(*)c1O ; *c1nnn(*)c1O]

each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer comprising an amino acid residue selected from glycine, valine, citrulline, lysine, isoleucine, phenylalanine, methionine, asparagine, proline, alanine, leucine, tryptophan, and tyrosine;
A is a bond, —OC(═O)—*,

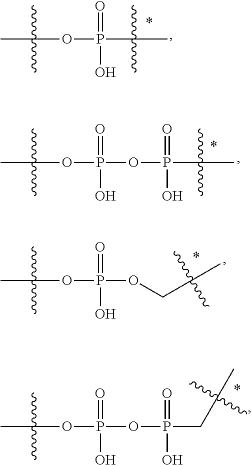 [SMILES: *OP(*)(=O)O ; *OP(=O)(O)OP(*)(=O)O ; *COP(=O)(O)O* ; *CP(=O)(O)OP(=O)(O)O*]

—C(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D;
L₃ is a spacer moiety having the structure

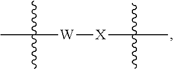

where
   W is —CH₂O—**, —CH₂N(R^{b})C(═O)O—**, —NHC(═O)C(R^{b})₂NHC(═O)O—**, —NHC(═O)C(R^{b})₂NH—**, —NHC(═O)C(R^{b})₂NHC(═O)—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
and
   the * of L₃ indicates the point of attachment to R².

Embodiment 37. The conjugate linker of any one of Embodiments 33 to 36, wherein:
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16,17,18,19, 20, 21, 22, 23, 24, 25, 26, 27, 28, 29 and 30;
Lp is a bivalent peptide spacer selected from

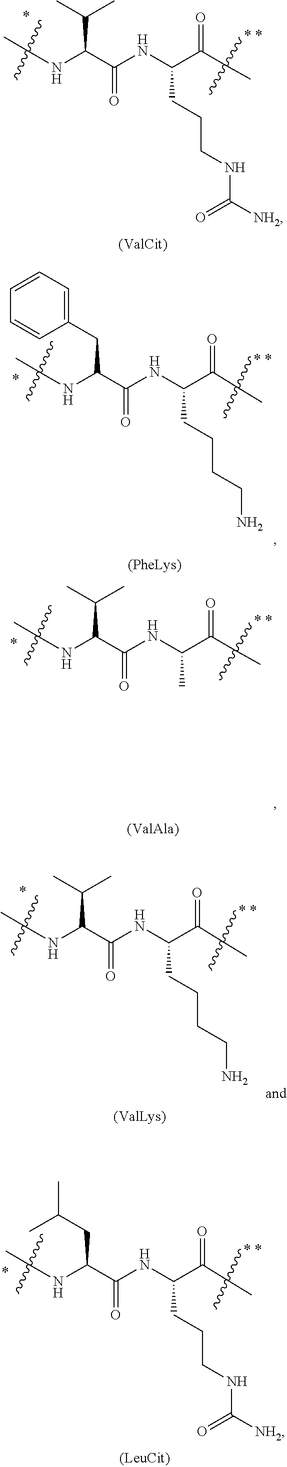

where the * of Lp indicates the attachment point to L₁;
L₃ is a spacer moiety having the structure

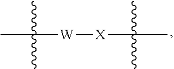

where
   W is —CH₂O—**, —CH₂N(R^{b})C(═O)O—**, —NHC(═O)CH₂NHC(═O)O—**, —NHC(═O)CH₂NH—**, —NHC(═O)CH₂NHC(═O)—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

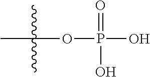 [SMILES: *OP(=O)(O)O]

groups;
and
A is a bond, —OC(═O)—*,

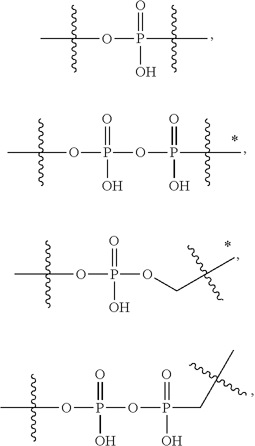 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D.

Embodiment 38. The conjugate linker of any one of Embodiments 33 to 37, wherein:
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

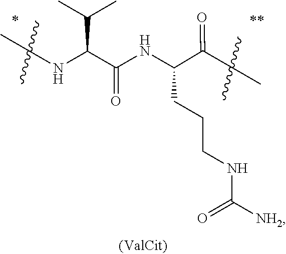 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of G;
L₃ is a spacer moiety having the structure

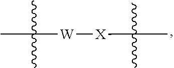

where
   W is —CH₂O—**, —CH₂N(Rb)C(═O)O—**, —NHC(═O)CH₂NHC(═O)O—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

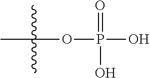 [SMILES: *OP(=O)(O)O]

groups;
and
A is a bond, —OC(═O)—*,

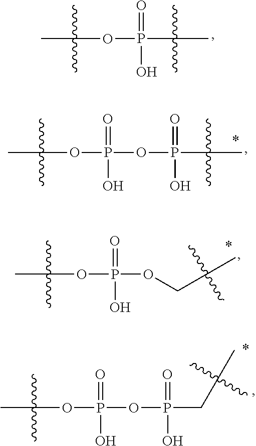 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D.

Embodiment 39. The conjugate linker of any one of Embodiments 33 to 38, wherein:
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

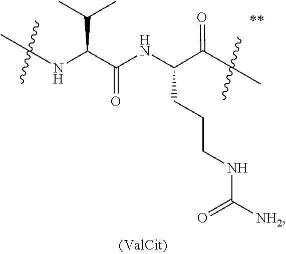 [SMILES: *N[C@H](C(=O)N[C@@H](CCCNC(N)=O)C(*)=O)C(C)C]

where the * of Lp indicates the attachment point to L₁;
L₃ is a spacer moiety having the structure

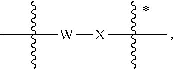

where
   W is —CH₂O—**, —CH₂N(Rb)C(═O)O—**, —NHC(═O)CH₂NHC(═O)O—**, —CH₂N(X—R²)C(═O)O—**, —C(═O)N(X—R²)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, or —NHC(═O)NH—**, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of Lₐ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

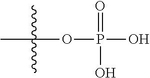 [SMILES: *OP(=O)(O)O]

groups;
and
A is a bond or —OC(═O)* in which * indicates the attachment point to D.

Embodiment 40. The conjugate linker of any one of Embodiments 33 to 39, wherein:
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

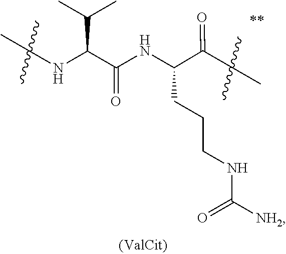 [SMILES: *N[C@H](C(=O)N[C@@H](CCCNC(N)=O)C(*)=O)C(C)C]

where the * of Lp indicates the attachment point to L₁;
L₃ is a spacer moiety having the structure

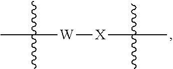

where
   W is —CH₂O—**, —CH₂N(Rb)C(═O)O—**, —NHC(═O)CH₂NHC(═O)O—**, —CH₂N(X—R²)C(═O)O—**, or —C(═O)N(X—R²)—**, wherein each R^{b} is independently selected from H, C₁-C₆alkyl or C₃-C₈cycloalkyl and wherein the ** of W indicates the point of attachment to X;
      X is ***—CH₂-triazolyl-*, wherein the *** of X indicates the point of attachment to W and the * of X indicates the point of attachment to R²;
      and
      the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

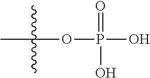 [SMILES: *OP(=O)(O)O]

groups;
and
A is a bond or —OC(═O)* in which * indicates the attachment point to D.

Embodiment 41. The conjugate linker of Formula (C′) having the structure having the structure of Formula (D′),

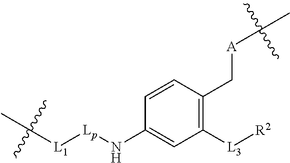

wherein
   L₁ is a bridging spacer;
   Lp is a bivalent peptide spacer;
   R² is a hydrophilic moiety;
   A is a bond, —OC(═O)—*,

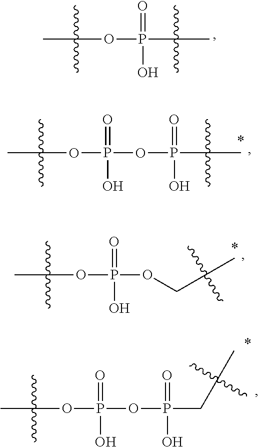 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D,
and
L₃ is a spacer moiety.

Embodiment 42. The conjugate linker of Embodiments 41, wherein:
L₁ is a bridging spacer;
Lp is a bivalent peptide spacer comprising two to four amino acid residues;
R² is a hydrophilic moiety;
A is a bond, —OC(═O)—*,

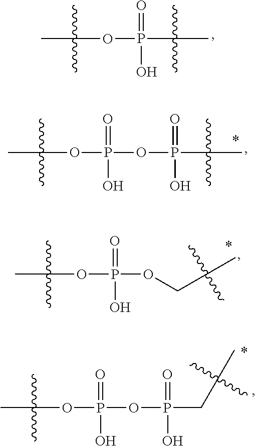 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D,
and
L₃ is a spacer moiety.

Embodiment 43. The conjugate linker of Embodiment 41 or 42, wherein:
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; *—C(═O)NH((CH₂)ₘO)ₜ(CH₂)ₙ—**; *—C(═O)O(CH₂)ₘSSC(R³)₂(CH₂)ₘC(═O)NR³(CH₂)ₘNR³C(═O)(CH₂)ₘ—**; *—C(═O)O(CH₂)ₘC(═O)NH(CH₂)ₘ—**; *—C(═O)(CH₂)ₘNH(CH₂)ₘ—**; *—C(═O)(CH₂)ₘNH(CH₂)ₙC(═O)—**; *—C(═O)(CH₂)ₘX₁(CH₂)ₘ—**; *—C(═O)((CH₂)ₘO)ₜ(CH₂)ₙX₁(CH₂)ₙ—**; *—C(═O)(CH₂)ₘNHC(═O)(CH₂)ₙ—**; *—C(═O)((CH₂)ₘO)ₜ(CH₂)ₙNHC(═O)(CH₂)ₙ—**; *—C(═O)(CH₂)ₘNHC(═O)(CH₂)ₙX₁(CH₂)ₙ—**; *—C(═O)((CH₂)ₘO)ₜ(CH₂)ₙNHC(═O)(CH₂)ₙX₁(CH₂)ₙ—**; *—C(═O)((CH₂)ₘO)ₜ(CH₂)ₙC(═O)NH(CH₂)ₘ—**; *—C(═O)(CH₂)ₘC(R³)₂—** or *—C(═O)(CH₂)ₘC(═O)NH(CH₂)ₘ—**, where the * of L₁ indicates the point of attachment to Lp;
R² is a hydrophilic moiety selected from polyethylene glycol, polyalkylene glycol, a sugar, an oligosaccharide, a polypeptide or C₂-C₆alkyl substituted with 1 to 3

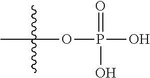 [SMILES: *OP(=O)(O)O]

groups;
each R³ is independently selected from H and C₁-C₆alkyl;
X₁ is

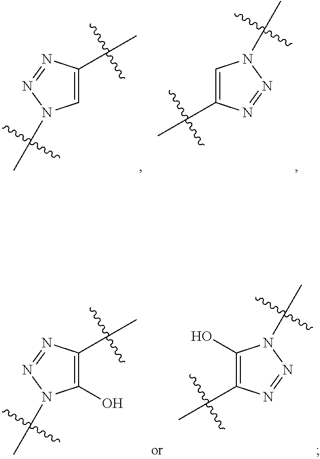 [SMILES: *c1cn(*)nn1 ; *c1cn(*)nn1 ; *c1nnn(*)c1O ; *c1nnn(*)c1O]

each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer comprising an amino acid residue selected from glycine, valine, citrulline, lysine, isoleucine, phenylalanine, methionine, asparagine, proline, alanine, leucine, tryptophan, and tyrosine; A is a bond, —OC(═O)—*,

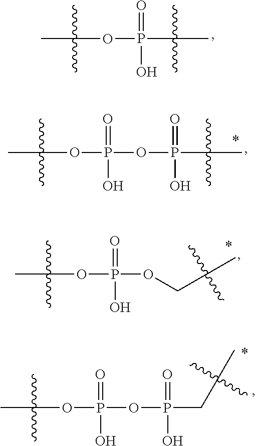 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D;
L₃ is a spacer moiety having the structure

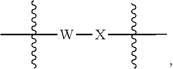

where
   W is —CH₂O—**, —CH₂N(Rb)C(═O)O—**, —NHC(═O)CH₂NHC(═O)O—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R².

Embodiment 44. The conjugate linker of any one of Embodiments 41 to 43, wherein:
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

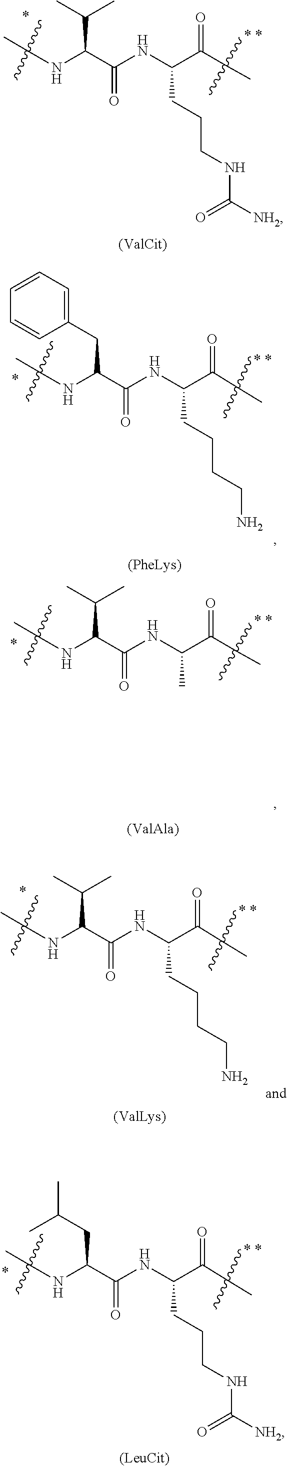

where the * of Lp indicates the attachment point to L₁ and the ** of Lp indicates the attachment point to the —NH— group of G;
L₃ is a spacer moiety having the structure

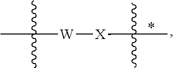

where
   W is —CH₂O—**, —CH₂N(Rb)C(═O)O—**, —NHC(═O)CH₂NHC(═O)O—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

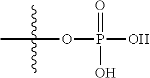 [SMILES: *OP(=O)(O)O]

groups;
and
A is a bond, —OC(═O)—*,

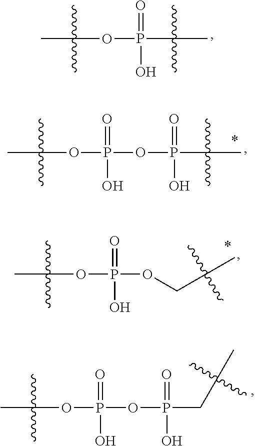 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D.

Embodiment 45. The conjugate linker of any one of Embodiments 41 to 44, wherein:
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

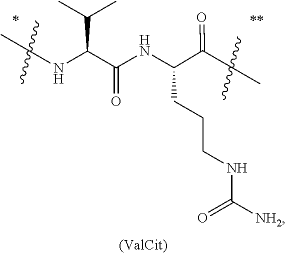 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of G;
L₃ is a spacer moiety having the structure

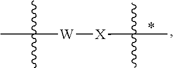

where
   W is —CH₂O—**, —CH₂N(Rb)C(═O)O—**, —NHC(═O)CH₂NHC(═O)O—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

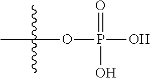 [SMILES: *OP(=O)(O)O]

groups;
and
A is a bond, —OC(═O)—*,

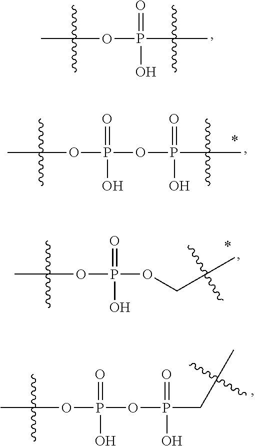 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D.

Embodiment 46. The conjugate linker of any one of Embodiments 41 to 45, wherein:
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

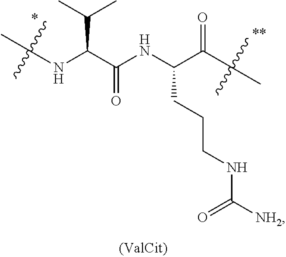 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of G;
L₃ is a spacer moiety having the structure

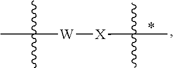

where
   W is —CH₂O—**, —CH₂N(Rb)C(═O)O—**, —NHC(═O)CH₂NHC(═O)O—**, —CH₂N(X—R²)C(═O)O—**, —C(═O)N(X—R²)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, or —NHC(═O)NH—**, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

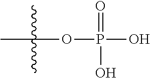 [SMILES: *OP(=O)(O)O]

groups;
and
A is a bond or —OC(═O)* in which * indicates the attachment point to D.

Embodiment 47. The conjugate linker of any one of Embodiments 41 to 46, wherein:
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

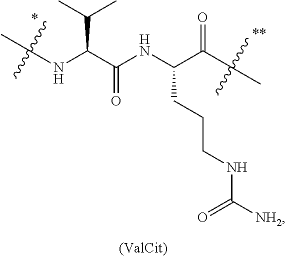 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of G;
L₃ is a spacer moiety having the structure

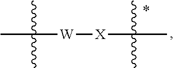

where
   W is —CH₂O—**, —CH₂N(Rb)C(═O)O—**, —NHC(═O)CH₂NHC(═O)O—**, —CH₂N(X—R²)C(═O)O—**, or —C(═O)N(X—R²)—**, wherein each R^{b} is independently selected from H, C₁-C₆alkyl or C₃-C₈cycloalkyl and wherein the ** of W indicates the point of attachment to X;
   X is ***—CH₂-triazolyl-*, wherein the *** of X indicates the point of attachment to W and the * of X indicates the point of attachment to R²
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

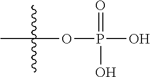 [SMILES: *OP(=O)(O)O]

groups;
and
A is a bond or —OC(═O)* in which * indicates the attachment point to D.

Embodiment 48. The conjugate linker of any one of Embodiments 33 to 47, having the structure:

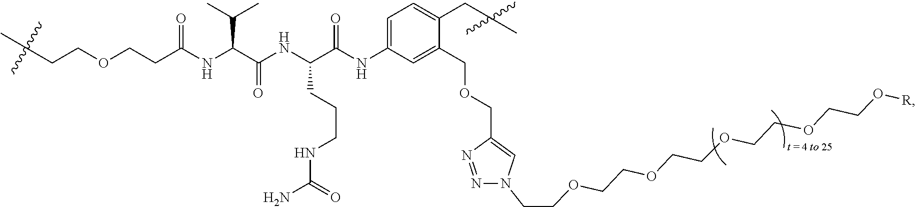

where R is H, —CH₃ or —CH₂CH₂C(═O)OH.

Embodiment 49. The conjugate linker of any one of Embodiments 33 to 47, having the structure:

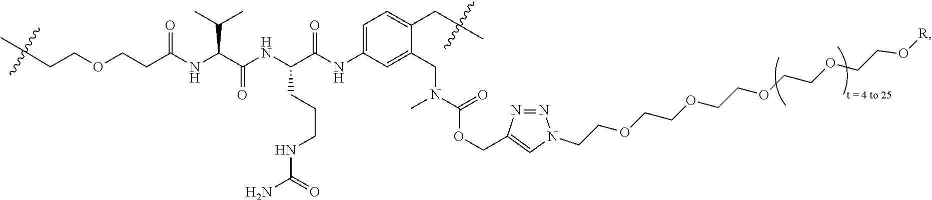

where R is H, —CH₃ or —CH₂CH₂C(═O)OH.

Embodiment 50. The conjugate linker of any one of Embodiments 33 to 47, having the structure:

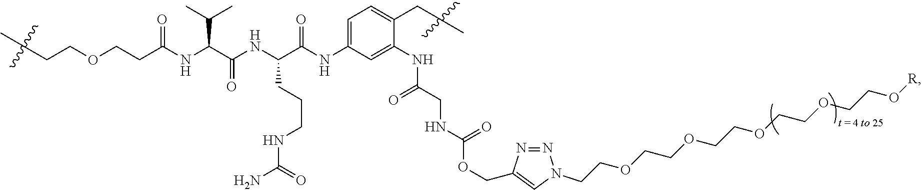

where R is H, —CH₃ or —CH₂CH₂C(═O)OH.

Embodiment 51. The conjugate linker of any one of Embodiments 33 to 47, having the structure:

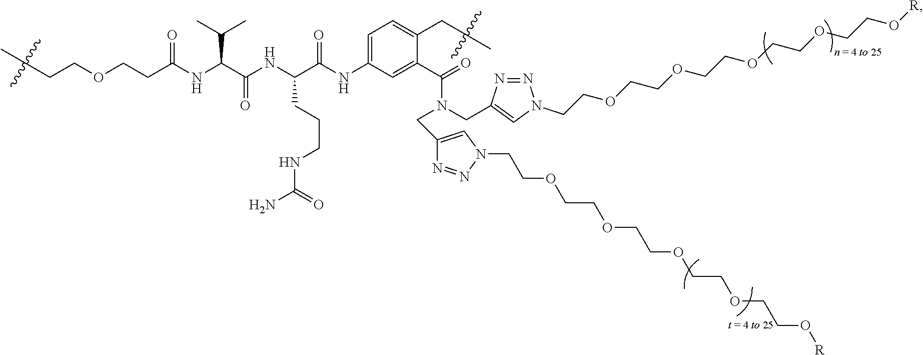

where each R is independently selected from H, —CH₃ or —CH₂CH₂C(═O)OH.

Embodiment 52. The conjugate linker of any one of Embodiments 37 to 47, having the structure:

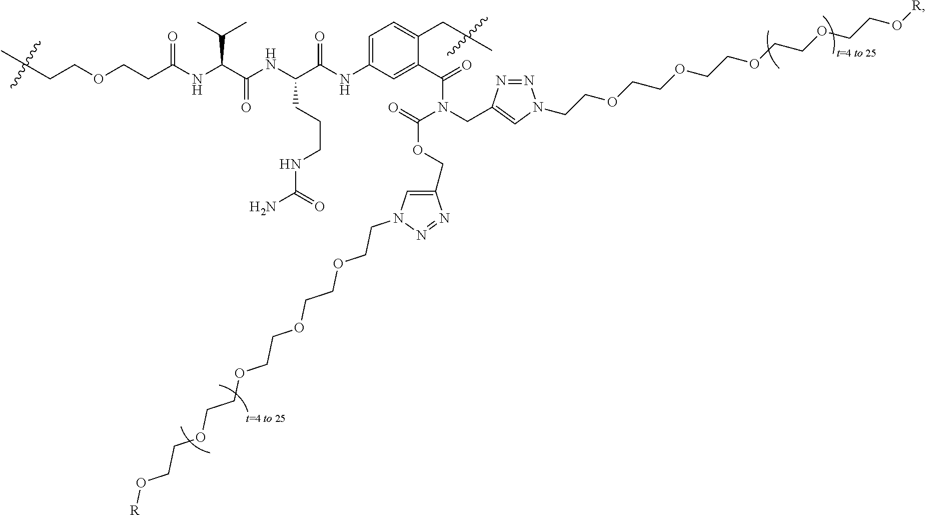

where each R is independently selected from H, —CH₃ or —CH₂CH₂C(═O)OH.

Embodiment 53. The conjugate linker of any one of Embodiments 33 to 47, having the structure:

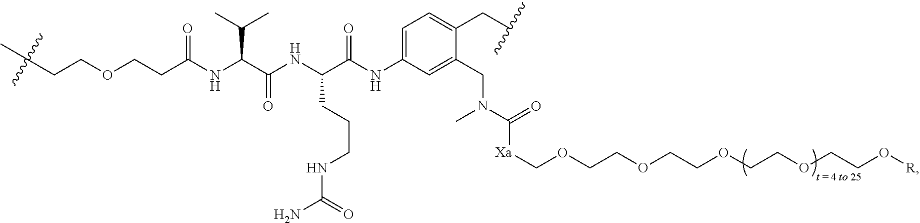

where Xa is —CH₂—, —OCH₂—, —NHCH₂— or —NRCH₂— and each R independently is H, —CH₃ or —CH₂CH₂C(═O)OH.

Embodiment 54. The conjugate linker of any one of Embodiments 33 to 47, having the structure:

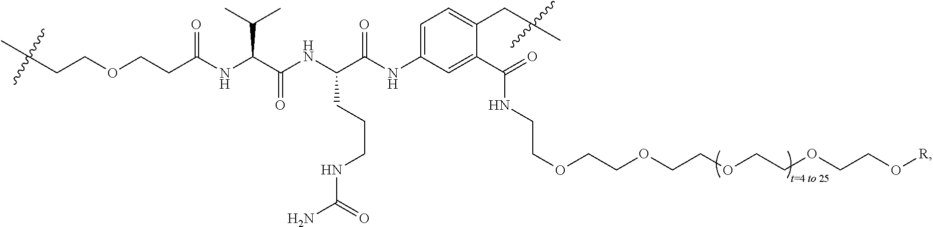

where R is H, —CH₃ or —CH₂CH₂C(═O)OH.

Embodiment 55. The conjugate linker of any one of Embodiments 33 to 47, having the structure:

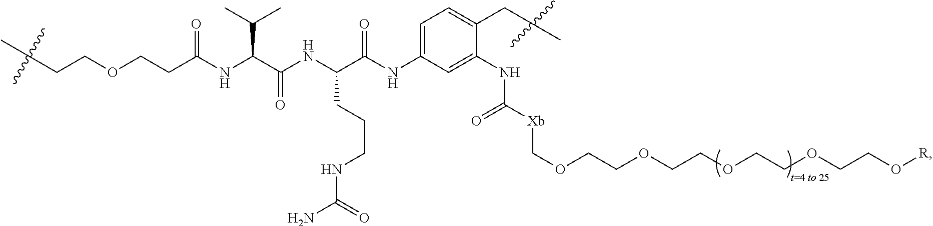

where Xb is —CH₂—, —OCH₂—, —NHCH₂— or —NRCH₂— and each R independently is H, —CH₃ or —CH₂CH₂C(═O)OH.

Embodiment 56. The conjugate linker of any one of Embodiments 33 to 47, having the structure:

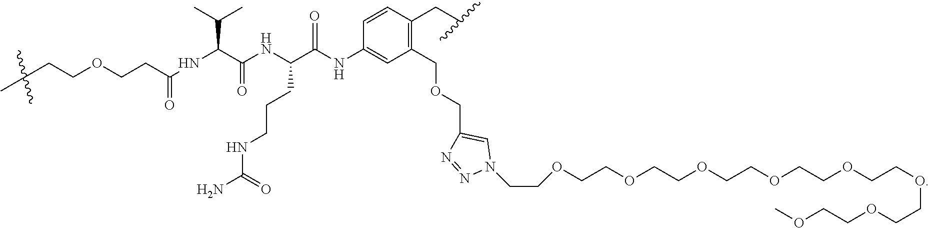 [SMILES: *CCOCCC(=O)N[C@H](C(=O)N[C@@H](CCCNC(N)=O)C(=O)Nc1ccc(C*)c(COCc2cn(CCOCCOCCOCCOCCOCCOCCOCCOC)nn2)c1)C(C)C]

Embodiment 57. The conjugate linker of any one of Embodiments 33 to 47, having the structure:

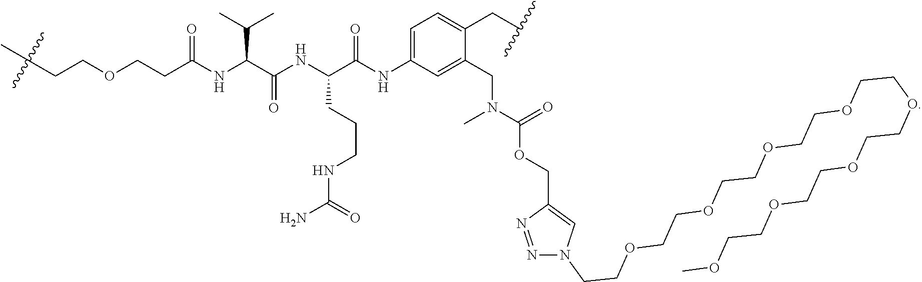 [SMILES: *CCOCCC(=O)N[C@H](C(=O)N[C@@H](CCCNC(N)=O)C(=O)Nc1ccc(C*)c(CN(C)C(=O)OCc2cn(CCOCCOCCOCCOCCOCCOCCOCCOC)nn2)c1)C(C)C]

Embodiment 58. The conjugate linker of any one of Embodiments 37 to 47, having the structure:

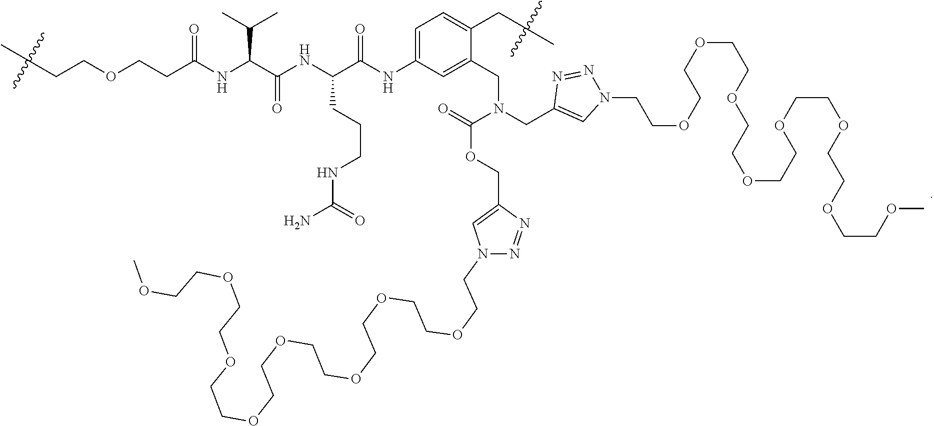 [SMILES: *CCOCCC(=O)N[C@H](C(=O)N[C@@H](CCCNC(N)=O)C(=O)Nc1ccc(C*)c(CN(Cc2cn(CCOCCOCCOCCOCCOCCOCCOCCOC)nn2)C(=O)OCc2cn(CCOCCOCCOCCOCCOCCOCCOCCOC)nn2)c1)C(C)C]

Embodiment 59. The conjugate linker of any one of Embodiments 33 to 47, having the structure:

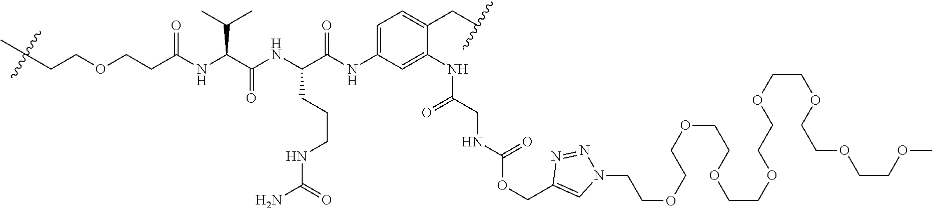 [SMILES: *CCOCCC(=O)N[C@H](C(=O)N[C@@H](CCCNC(N)=O)C(=O)Nc1ccc(C*)c(NC(=O)CNC(=O)OCc2cn(CCOCCOCCOCCOCCOCCOCCOCCOC)nn2)c1)C(C)C]

Embodiment 60. The conjugate linker of any one of Embodiments 33 to 47, having the structure:

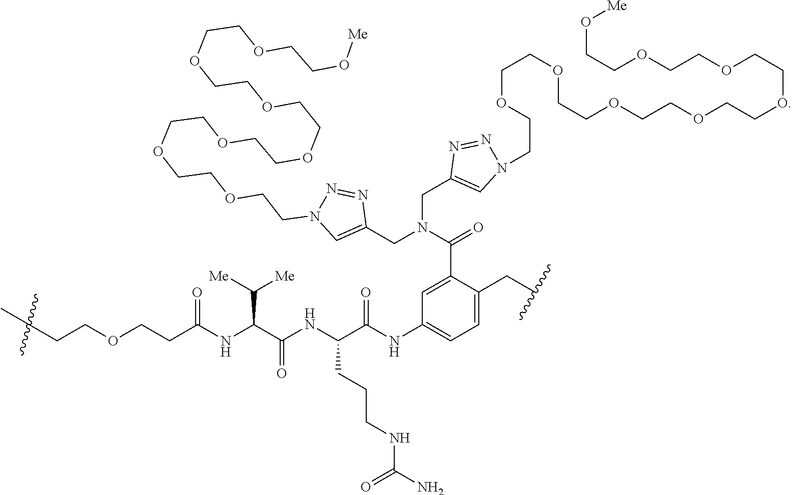 [SMILES: *CCOCCC(=O)N[C@H](C(=O)N[C@@H](CCCNC(N)=O)C(=O)Nc1ccc(C*)c(C(=O)N(Cc2cn(CCOCCOCCOCCOCCOCCOCCOCCOC)nn2)Cc2cn(CCOCCOCCOCCOCCOCCOCCOCCOC)nn2)c1)C(C)C]

Embodiment 61. The conjugate linker of any one of Embodiments 33 to 47, having the structure:

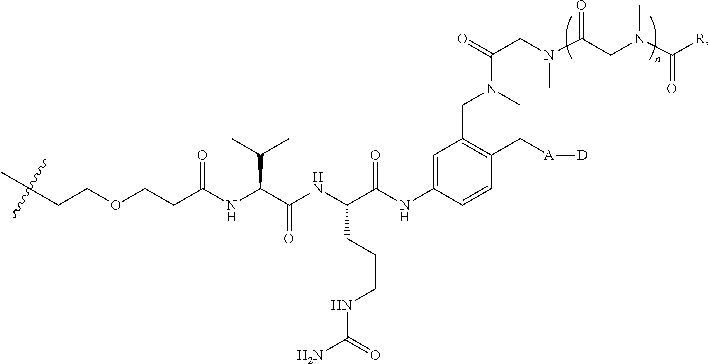

where n is an integer between 2 and 24.

For illustrative purposes, the general reaction schemes depicted herein provide potential routes for synthesizing the compounds of the present invention as well as key intermediates. For a more detailed description of the individual reaction steps, see the Examples section below. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

By way of example, a general synthesis for compounds of Formula (B′) is shown below in Scheme 1.

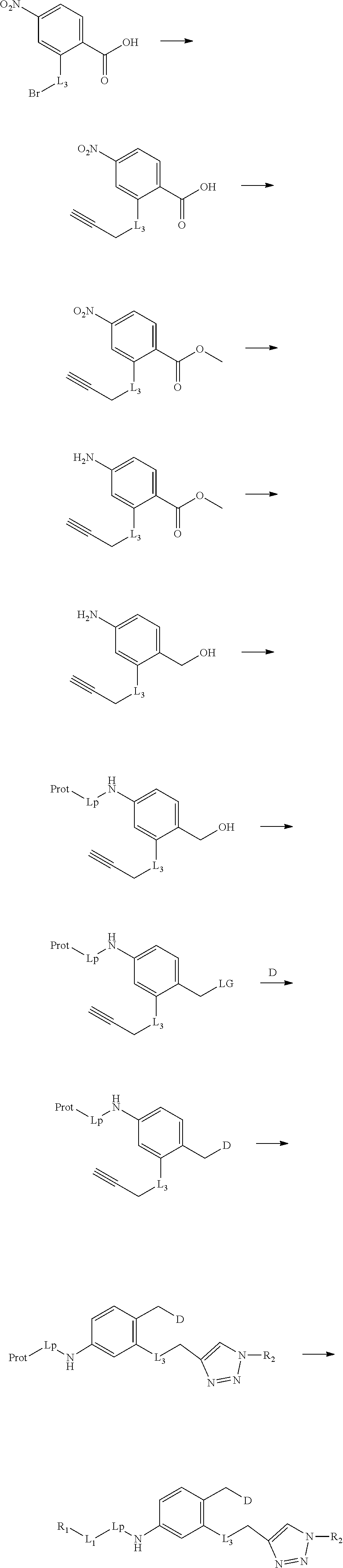

### Antibody Drug Conjugates of the Invention

The present invention provides Antibody Drug Conjugates, also referred to herein as immunoconjugates, which comprise conjugate linkers which comprise one or more hydrophilic moieties.

The Antibody Drug Conjugates of the invention have the structure of Formula (E′):

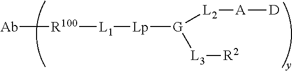

wherein:
   Ab is an antibody (e.g., anti-EphA2 antibody or anti-B7-H3 antibody) or fragment thereof;
   R¹⁰⁰ is a coupling group;
   L₁ is a bridging spacer;
   Lp is a bivalent peptide spacer;
   G-L₂-A is a self-immolative spacer;
   R² is a hydrophilic moiety;
   L₂ is a bond, a methylene, a neopentylene or a C₂-C₃alkenylene;
   A is a bond, —OC(═O)—*,

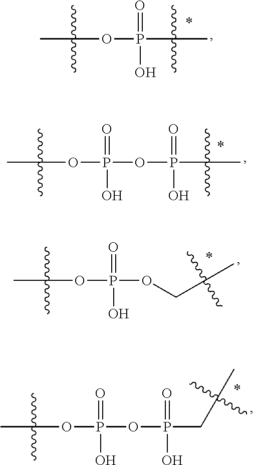 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D;
L₃ is a spacer moiety;
D is a Drug moiety as defined herein, e.g., a panRAS inhibitor, and may comprise an N, wherein D can be connected to A via a direct bond from A to the N of the Drug moiety,
and
y is 1, 2, 3, 4, 5, 6, 7, 8, 9, 10, 11, 12, 13, 14, 15 or 16.

Certain aspects and examples of the Antibody Drug Conjugates of the invention are provided in the following listing of enumerated embodiments. It will be recognized that features specified in each embodiment may be combined with other specified features to provide further embodiments of the present invention.

Embodiment 62. The immunoconjugate of Formula (E′) wherein:
Ab is an antibody (e.g., anti-EphA2 antibody or anti-B7-H3 antibody) or fragment thereof;
R¹⁰⁰ is a coupling group;
L₁ is a bridging spacer;
Lp is a bivalent peptide spacer comprising two to four amino acid residues;
G-L₂-A is a self-immolative spacer;
R² is a hydrophilic moiety;
L₂ is a bond, a methylene, a neopentylene or a C₂-C₃alkenylene;
A is a bond, —OC(═O)—*,

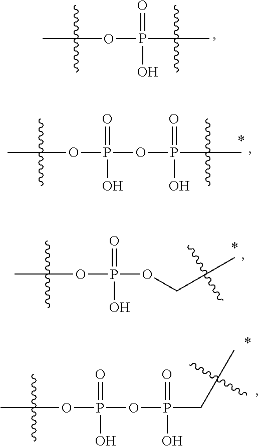 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D;
L₃ is a spacer moiety;
D is a Drug moiety as defined herein wherein D is connected to A via a direct bond from A to D (e.g., an N of the Drug moiety),
and
y is 1, 2, 3, 4, 5, 6, 7, 8, 9, 10, 11, 12, 13, 14, 15 or 16.

Embodiment 63. The immunoconjugate of Formula (E′) or Embodiment 62, wherein:
Ab is an antibody (e.g., anti-EphA2 antibody or anti-B7-H3 antibody) or fragment thereof;
R¹⁰⁰ is a coupling group;
L₁ is a bridging spacer;
Lp is a bivalent peptide spacer comprising two to four amino acid residues;
the

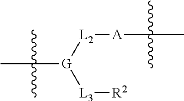

group is selected from:

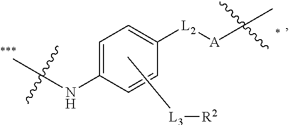

wherein the * of

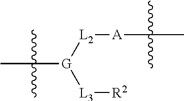

indicates the point of attachment to D (e.g., to an N or a O of the Drug moiety), the *** of

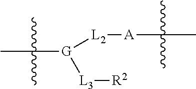

indicates the point of attachment to Lp;
R² is a hydrophilic moiety;
L₂ is a bond, a methylene, a neopentylene or a C₂-C₃alkenylene;
A is a bond, —OC(═O)—*,

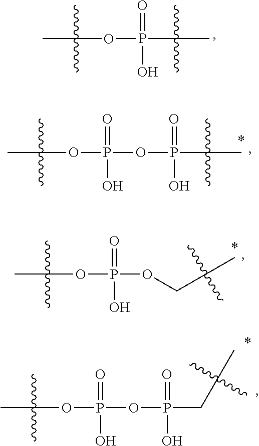 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C1-C₆ alkyl, and C₃-C₈ cycloalkyl and the * of A indicates the point of attachment to D;
L₃ is a spacer moiety;
D is a Drug moiety as defined herein and comprising an N, wherein D is connected to A via a direct bond from A to the N of the Drug moiety,
and
y is 1, 2, 3, 4, 5, 6, 7, 8, 9, 10, 11, 12, 13, 14, 15 or 16.

Embodiment 64. The immunoconjugate of Formula (E′) or any one of Embodiments 62 to 63 having the structure of Formula (F′),

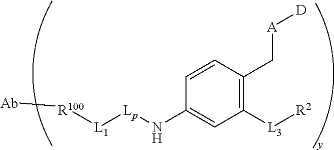

wherein:
   Ab is an antibody (e.g., anti-EphA2 antibody or anti-B7-H3 antibody) or fragment thereof;
   R¹⁰⁰ is a coupling group;
L₁ is a bridging spacer;
Lp is a bivalent peptide spacer comprising two to four amino acid residues;
R² is a hydrophilic moiety;
A is a bond, —OC(═O)—*,

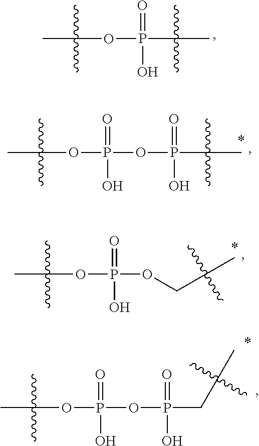 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D;
L₃ is a spacer moiety;
D is a Drug moiety as defined herein and comprising an N, wherein D is connected to A via a direct bond from A to the N of the Drug moiety,
and
y is 1, 2, 3, 4, 5, 6, 7, 8, 9, 10, 11, 12, 13, 14, 15 or 16.

Embodiment 65. The immunoconjugate of Formula (D′) or any one of Embodiments 62 to 64, wherein:
Ab is an antibody (e.g., anti-EphA2 antibody or anti-B7-H3 antibody) or fragment thereof;
R¹⁰⁰ is

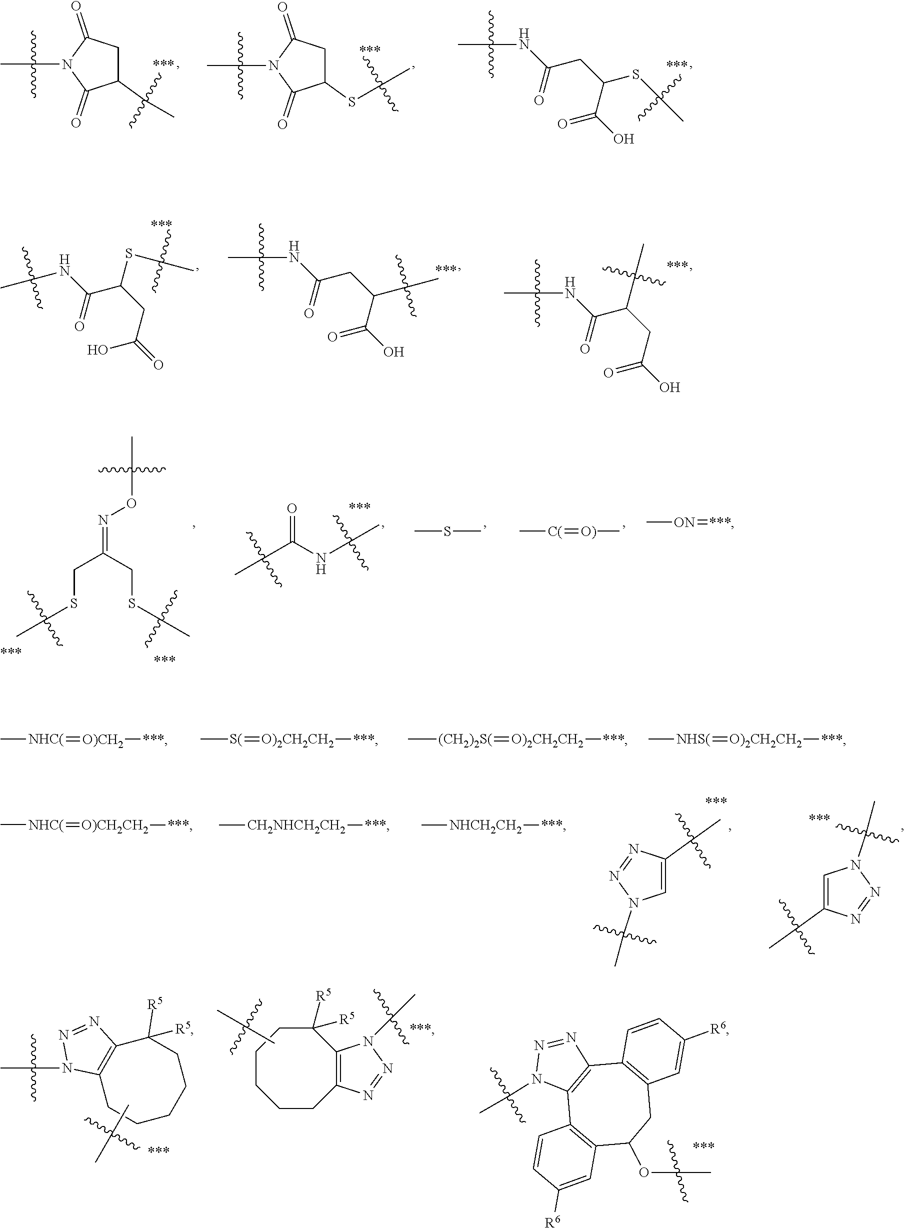

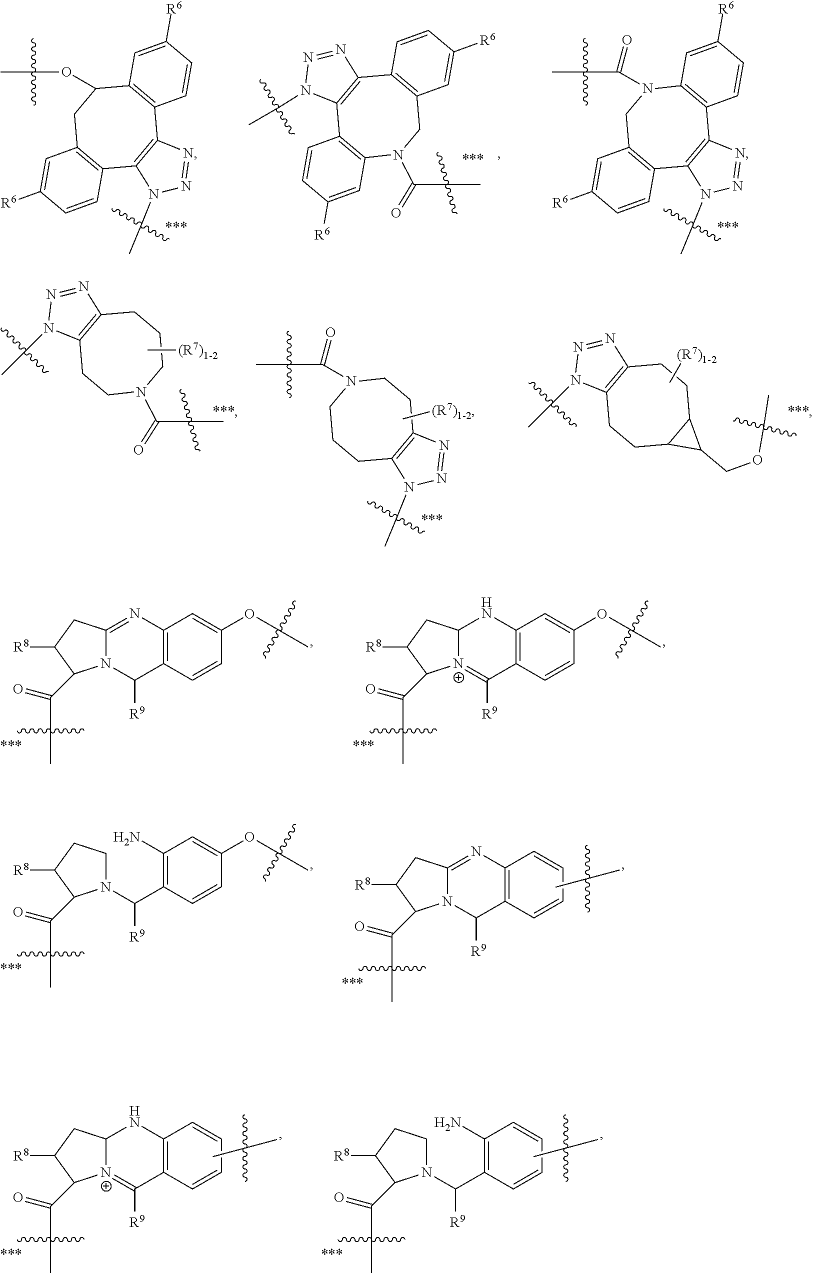

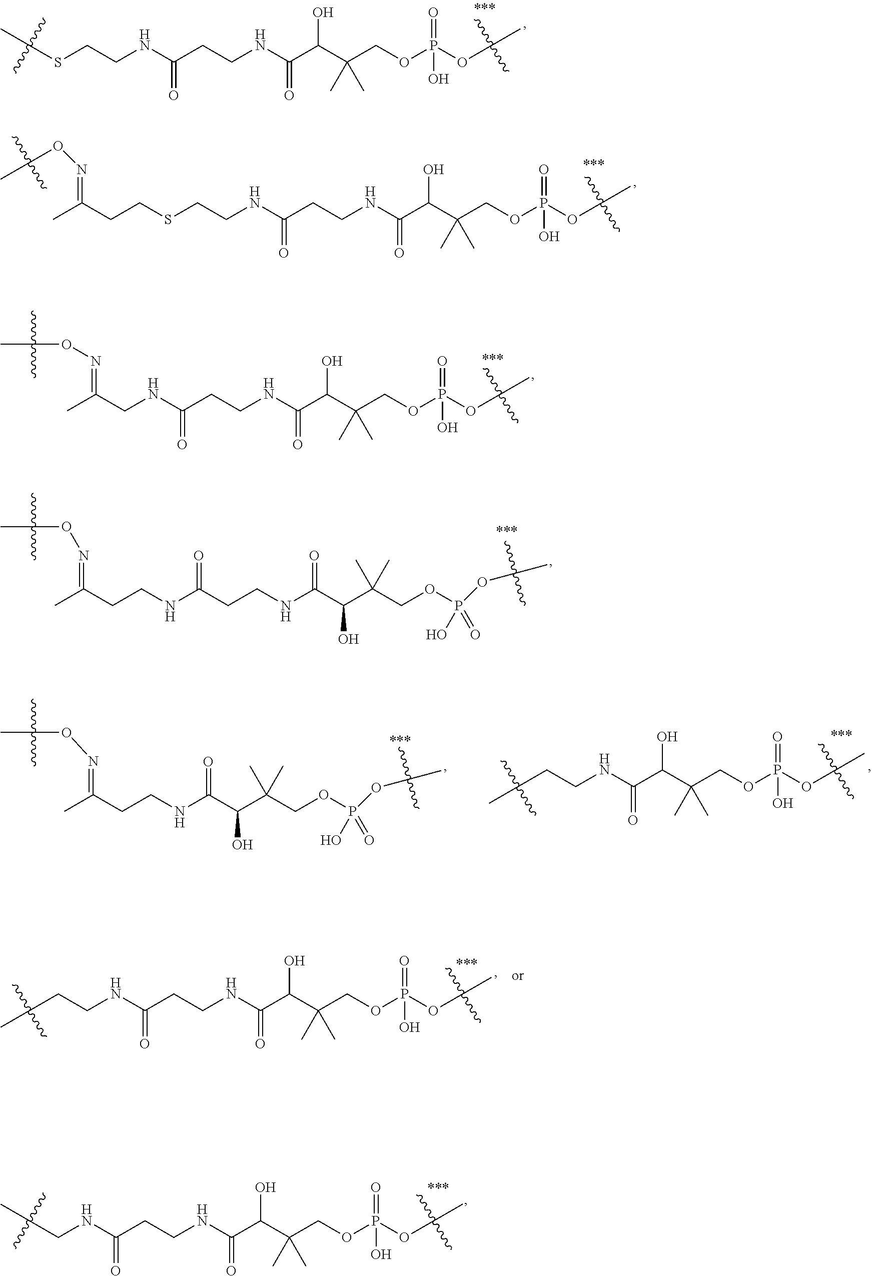

where the *** of R¹⁰⁰ indicates the point of attachment to Ab;
L₁ is *—C(═O)(CH₂)ₘO(CH₂)ₘ—**; * C(═O)((CH)ₘO)(CH)ₘ—**; *C(═O)NH((CH₂)ₘO)*(CH₂)ₙ**; *—C(═O)O(CH₂)ₘSSC(R³)₂(CH₂)ₘC(═O)NR³(CH₂)ₘNR³C(O)(CH₂)ₘ**; *—C(═O)O(CH₂)ₘC(═O)NH(CH₂)ₘ—**; *—C(═O)(CH)N H(CH)ₘ—**; *—C(═O)(CH₂)ₘNH(CH₂)ₙC(═O)—**; *—C(═O)(CH₂)ₘX₁(CH₂)ₘ—**; *—C(═O)((CH₂)ₘO)ₜ(CH₂)ₙX₁(CH₂)ₙ**; *C(═O)(CH₂)ₘN—HC(═O)(CH₂)ₙ—**; *—C(═O)((CH₂)ₘO)ₜ(CH₂)ₙNHC(═O)(CH₂)ₙ**; *—C(═O)(CH₂)ₘNHC(═O)(CH₂)ₙX₁(CH₂)ₙ—**; *—C(═O)((CH₂)ₘO)ₜ(CH₂)ₙNHC(═O)(CH₂)ₙX₁(CH₂)ₙ—**; *—C(═O)((CH₂)ₘO)ₜ(CH₂)ₙC(═O)NH(CH₂)ₘ—**; *—C(═O)(CH₂)ₘC(R³)₂—** or *—C(═O)(CH₂)ₘC(═O)NH(CH₂)ₘ—**, where the * of L₁ indicates the point of attachment to Lp, and the ** of L₁ indicates the point of attachment to R¹⁰⁰;
R² is a hydrophilic moiety selected from polyethylene glycol, polyalkylene glycol, a sugar, an oligosaccharide, a polypeptide or C₂-C₆alkyl substituted with 1 to 3

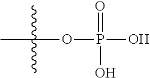 [SMILES: *OP(=O)(O)O]

groups;
each R³ is independently selected from H and C₁-C₆alkyl;
R⁴ is 2-pyridyl or 4-pyridyl;
each R⁵ is independently selected from H, C₁-C₆alkyl, F, Cl, and —OH;
each R⁶ is independently selected from H, C₁-C₆alkyl, F, Cl, —NH₂, —OCH₃, —OCH₂CH₃, —N(CH₃)₂, —CN, —NO₂ and —OH;
each R⁷ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
X is

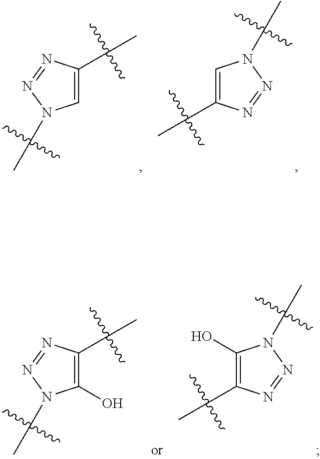 [SMILES: *c1cn(*)nn1 ; *c1cn(*)nn1 ; *c1nnn(*)c1O ; *c1nnn(*)c1O]

each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer comprising an amino acid residue selected from valine, citrulline, lysine, isoleucine, phenylalanine, methionine, asparagine, proline, alanine, leucine, tryptophan, and tyrosine;
A is a bond, —OC(═O)—*,

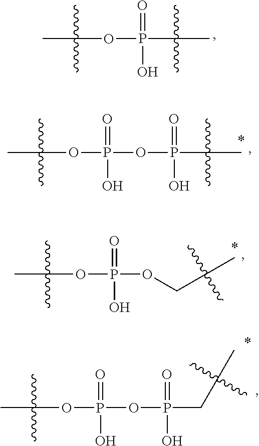 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D;
L₃ is a spacer moiety having the structure

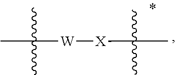

where
   W is —CH₂O—**, —CH₂N(Rb)C(═O)O—**, —NHC(═O)C(R^{b})₂NHC(═O)O—**, —NHC(═O)C(R^{b})₂NH—**, —NHC(═O)C(R^{b})₂NHC(═O)—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
D is a Drug moiety as defined herein and comprising an N, wherein D is connected to A via a direct bond from A to the N of the Drug moiety,
and
y is 1, 2, 3, 4, 5, 6, 7, 8, 9, 10, 11, 12, 13, 14, 15 or 16.

Embodiment 66. The immunoconjugate of Formula (D′) or any one of Embodiments 62 to 65, wherein:
Ab is an antibody (e.g., anti-EphA2 antibody or anti-B7-H3 antibody) or fragment thereof;
R¹⁰⁰ is

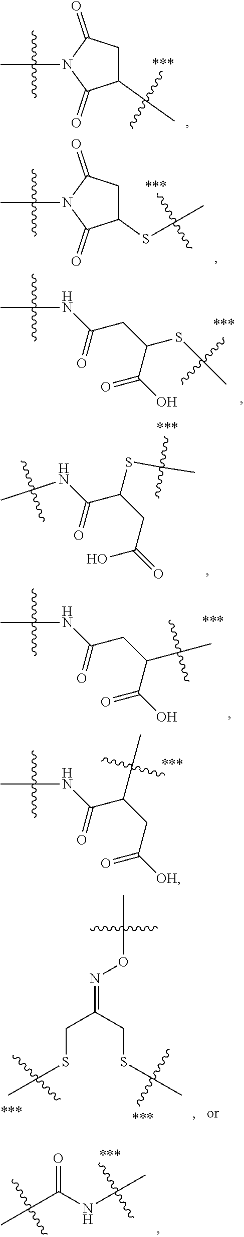

where the *** of R¹⁰⁰ indicates the point of attachment to Ab;
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp, and the ** of L₁ indicates the point of attachment to R¹⁰⁰;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

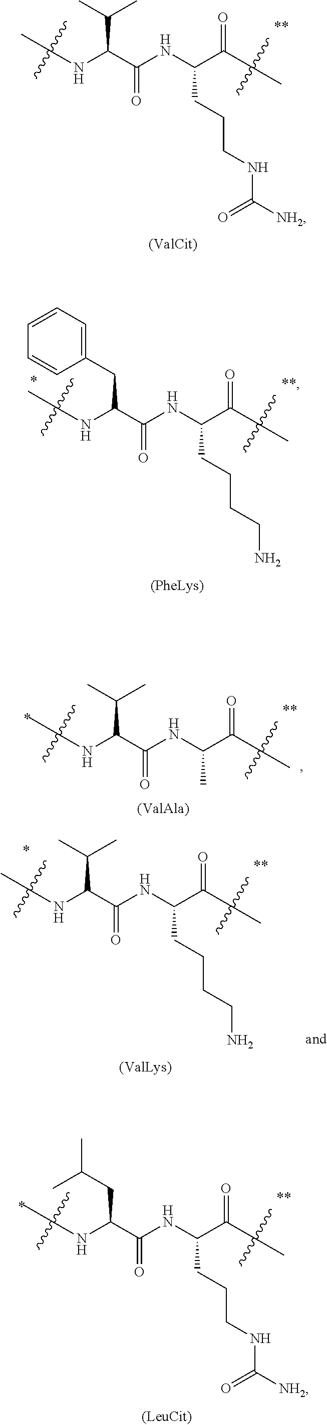

where the * of Lp indicates the attachment point to L₁ and the ** of Lp indicates the attachment point to the —NH— group of G;
L₃ is a spacer moiety having the structure

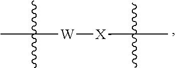

where
   W is —CH₂O—**, —CH₂N(Rb)C(═O)O—**, —NHC(═O)CH₂NHC(═O)O—**, —NHC(═O)CH₂NH—**, —NHC(═O)CH₂NHC(═O)—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

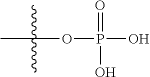 [SMILES: *OP(=O)(O)O]

groups;
A is a bond, —OC(═O)—*,

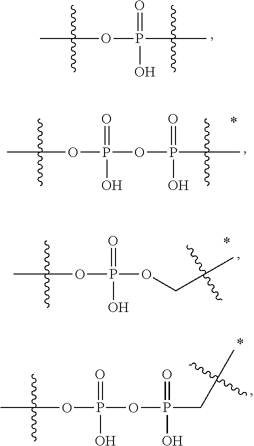 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D;
D is a Drug moiety as defined herein and comprising an N or an O, wherein D is connected to A via a direct bond from A to the N or the O of the Drug moiety,
and
y is 1, 2, 3, 4, 5, 6, 7, 8, 9, 10, 11, 12, 13, 14, 15 or 16.

Embodiment 67. The immunoconjugate of Formula (E′) or any one of Embodiments 62 to 66, wherein:
Ab is an antibody (e.g., anti-EphA2 antibody or anti-B7-H3 antibody) or fragment thereof;
R¹⁰⁰ is

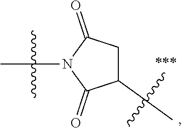 [SMILES: *C1CC(=O)N(*)C1=O]

where the *** of R¹⁰⁰ indicates the point of attachment to Ab;
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp, and the ** of L₁ indicates the point of attachment to R¹⁰⁰;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

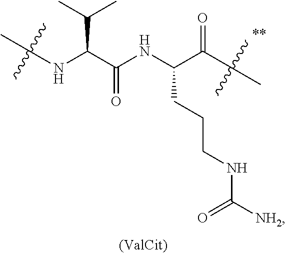 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of G;
L₃ is a spacer moiety having the structure

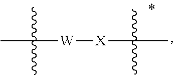

where
   W is —CH₂O—**, —CH₂N(Rb)C(═O)O—**, —NHC(═O)CH₂NHC(═O)O—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3 A is a bond, —OC(═O)—*,

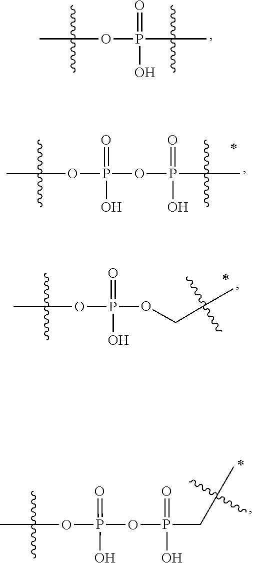 [SMILES: *OP(*)(=O)O ; *OP(=O)(O)OP(*)(=O)O ; *COP(=O)(O)O* ; *CP(=O)(O)OP(=O)(O)O*]

—OC(═O)N(CH₃)CH₂CH₂N(CH₃)C(═O)—* or —OC(═O)N(CH₃)C(R^{a})₂C(R^{a})₂N(CH₃)C(═O)—*, wherein each R^{a} is independently selected from H, C₁-C₆ alkyl, and C₃-C₈ cycloalkyl and the * of A indicates the point of attachment to D;
D is a Drug moiety as defined herein and comprising an N or an O, wherein D is connected to A via a direct bond from A to the N or the O of the Drug moiety,
and
y is 1, 2, 3, 4, 5, 6, 7, 8, 9, 10, 11, 12, 13, 14, 15 or 16.

Embodiment 68. The immunoconjugate of Formula (E′) or any one of Embodiments 62 to 67, wherein:
Ab is an antibody (e.g., anti-EphA2 antibody or anti-B7-H3 antibody) or fragment thereof;
R¹⁰⁰ is

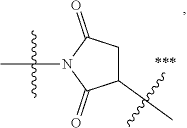 [SMILES: *C1CC(=O)N(*)C1=O]

where the *** of R¹⁰⁰ indicates the point of attachment to Ab;
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp and the ** of L₁ indicates the point of attachment to R¹⁰⁰;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

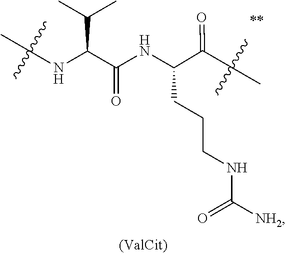 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of G;
L₃ is a spacer moiety having the structure

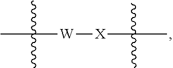

where
   W is —CH₂O—**, —CH₂N(Rb)C(═O)O—**, —NHC(═O)CH₂NHC(═O)O—**, —CH₂N(X—R²)C(═O)O—**, —C(═O)N(X—R²)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, or —NHC(═O)NH—**, wherein each R^{b} is independently selected from H, C₁-C₆alkyl or C₃-C₈cycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

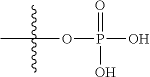 [SMILES: *OP(=O)(O)O]

groups;
A is a bond or —OC(═O)* in which * indicates the attachment point to D;
D is a Drug moiety as defined herein and comprising an N or an O, wherein D is connected to A via a direct bond from A to the N or the O of the Drug moiety, and
y is 1, 2, 3, 4, 5, 6, 7, 8, 9, 10, 11, 12, 13, 14, 15 or 16.

Embodiment 69. The immunoconjugate of Formula (E′) or any one of Embodiments 62 to 68, wherein:
Ab is an antibody (e.g., anti-EphA2 antibody or anti-B7-H3 antibody) or fragment thereof;

R¹⁰⁰ is

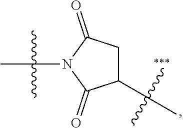 [SMILES: *C1CC(=O)N(*)C1=O]

where the *** of R¹⁰⁰ indicates the point of attachment to Ab;
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; or *—C(═O)NH((CH₂)ₘO)ₜ(CH₂)ₙ—, where the * of L₁ indicates the point of attachment to Lp and the ** of L₁ indicates the point of attachment to R¹⁰⁰;
each m is independently selected from 1, 2, 3, 4, 5, 6, 7, 8, 9 and 10;
each n is independently selected from 1, 2, 3, 4, 5, 6, 7, 8, 9 and 10;
each t is independently selected from 2, 3, 4, 5, 6, 7, 8, 9, 10, 11, 12, 13, 14, 15, 16, 17, 18, 19, 20, 21, 22, 23, 24, 25, 26, 27, 28, 29 and 30;
Lp is a bivalent peptide spacer selected from

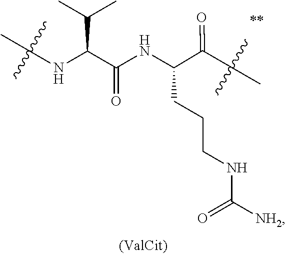 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of G;
L₃ is a spacer moiety having the structure

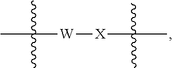

where
   W is —CH₂O—**, —CH₂N(Rb)C(═O)O—**, —NHC(═O)CH₂NHC(═O)O—**, —CH₂N(X—R²)C(═O)O—**, or —C(═O)N(X—R²)—**, wherein each R^{b} is independently selected from H, C₁-C₆alkyl or C₃-C₈ acycloalkyl and wherein the ** of W indicates the point of attachment to X;
   X is ***—CH₂-triazolyl-*, wherein the *** of X indicates the point of attachment to W and the * of X indicates the point of attachment to R²
   and
   the * of L₃ indicates the point of attachment to R²;
R² is a hydrophilic moiety selected from polyethylene glycol, polyalkylene glycol, a sugar, an oligosaccharide, a polypeptide or C₂-C₆alkyl substituted with 1 to 3

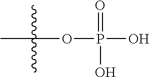 [SMILES: *OP(=O)(O)O]

groups;
A is a bond or —OC(═O)* in which * indicates the attachment point to D;
D is a Drug moiety as defined herein and comprising an N or an O, wherein D is connected to A via a direct bond from A to the N or the O of the Drug moiety,
and
y is 1,2,3,4,5,6,7,8,9,10,11,12,13,14, 15 or 16.

Embodiment 70. The immunoconjugate of Formula (E′) or any one of Embodiments 62 to 65, wherein
R¹⁰⁰ is

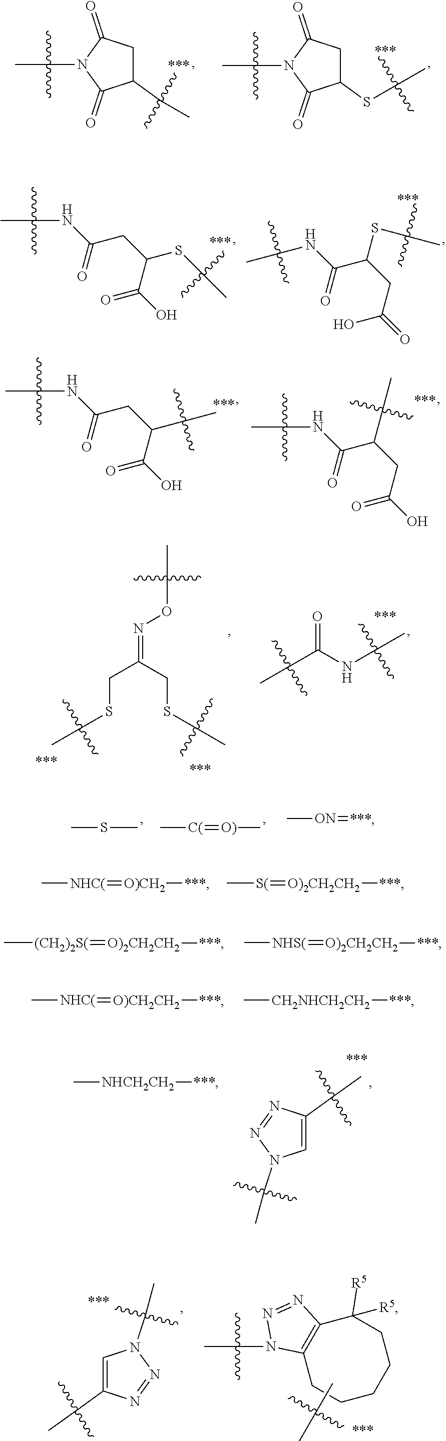

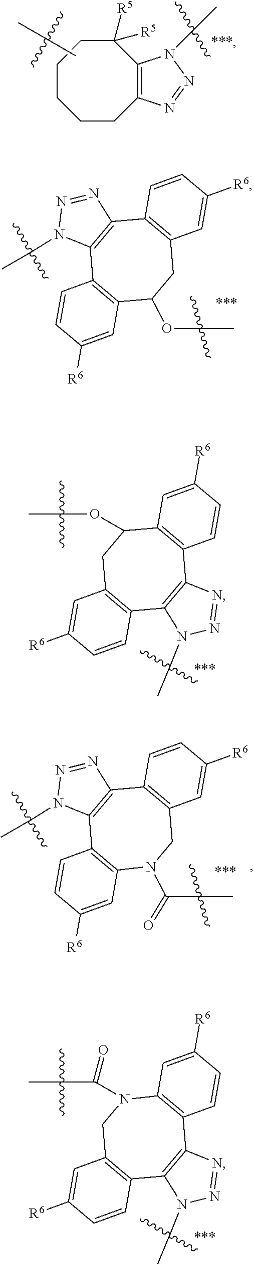

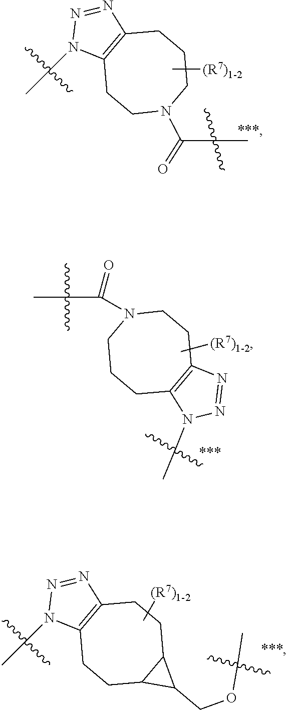

where the *** of R¹⁰⁰ indicates the point of attachment to Ab.

Embodiment 71. The immunoconjugate of Formula (E′) or any one of Embodiments 60 to 63, wherein
R¹⁰⁰ is

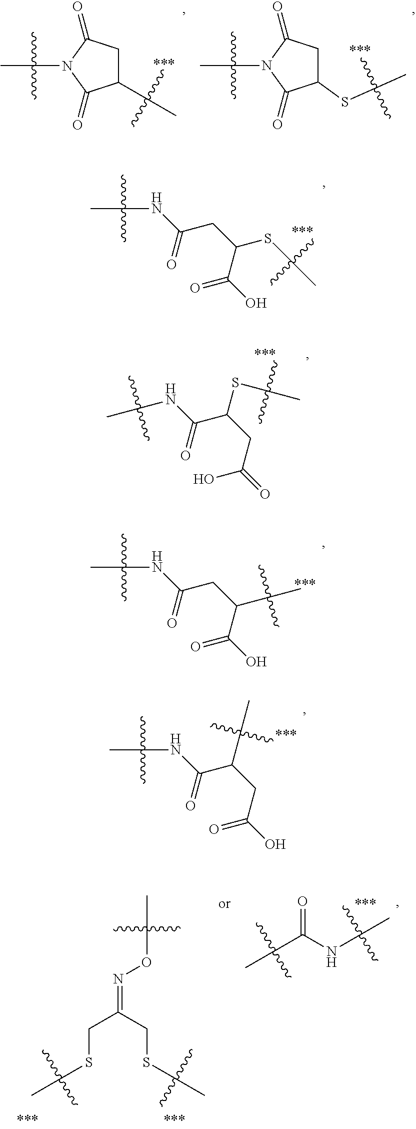

where the *** of R¹⁰⁰ indicates the point of attachment to Ab.

Embodiment 72. The immunoconjugate of Formula (E′) or any one of Embodiments 62 to 65, wherein
R¹⁰⁰ is

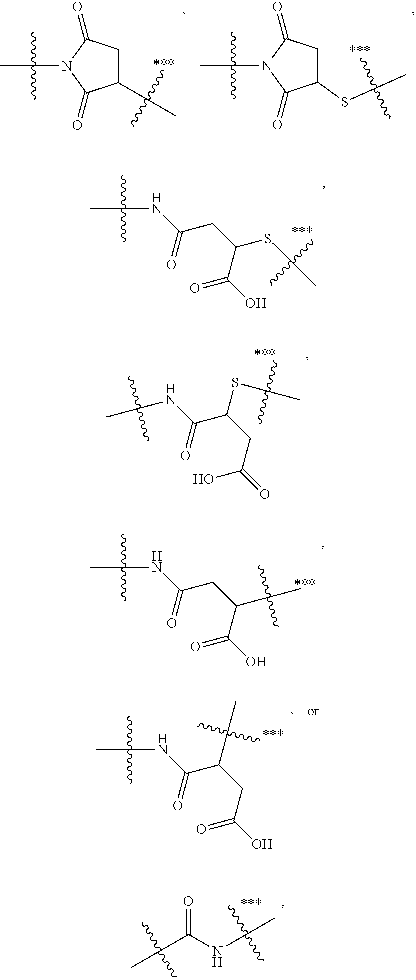

where the *** of R¹⁰⁰ indicates the point of attachment to Ab.

Embodiment 73. The immunoconjugate of Formula (E′) or any one of Embodiments 62 to 72 having the structure:

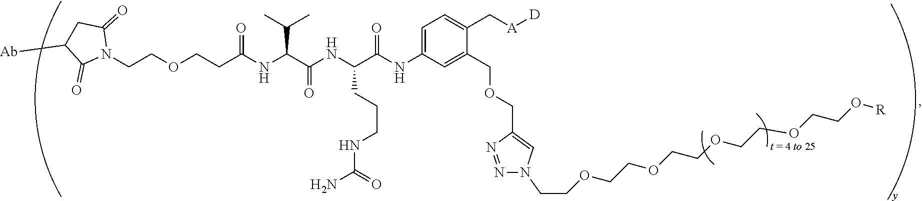

where
R is H, —CH₃ or —CH₂CH₂C(═O)OH and y is 1, 2, 3, 4, 5, 6, 7, 8, 9, 10, 11, 12, 13, 14, 15 or 16.

Embodiment 74. The immunoconjugate of Formula (E′) or any one of Embodiments 62 to 72 having the structure:

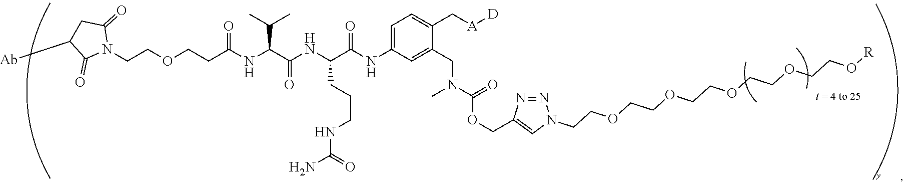

where
R is H, —CH₃ or —CH₂CH₂C(═O)OH and y is 1, 2, 3, 4, 5, 6, 7, 8, 9, 10, 11, 12, 13, 14, 15 or 16.

Embodiment 75. The immunoconjugate of Formula (E′) or any one of Embodiments 62 to 72 having the structure:

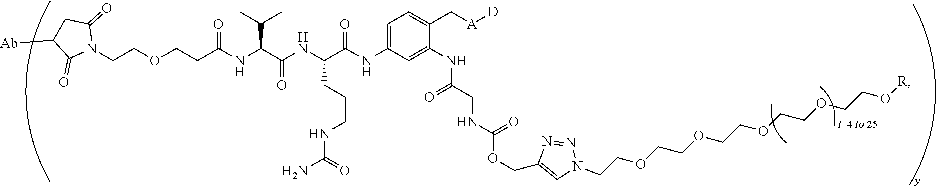

where R is H, —CH₃ or —CH₂CH₂C(═O)OH and y is 1, 2, 3, 4, 5, 6, 7, 8, 9, 10, 11, 12, 13, 14, 15 or 16.

Embodiment 76. The immunoconjugate of Formula (E′) or any one of Embodiments 62 to 72 having the structure:

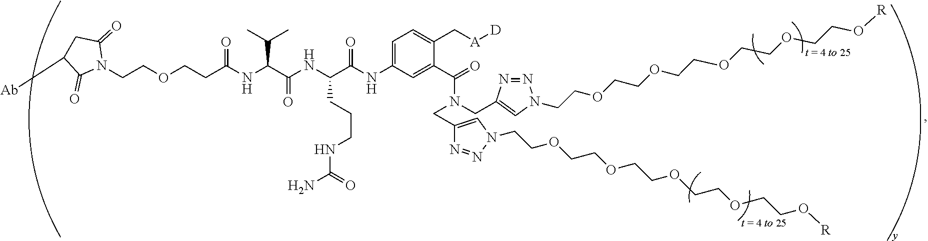

where each R is independently selected from H, —CH₃ or —CH₂CH₂C(═O)OH and y is 1, 2, 3, 4, 5, 6, 7, 8, 9, 10, 11, 12, 13, 14, 15 or 16.

Embodiment 77. The immunoconjugate of Formula (E′) or any one of Embodiments 62 to 72 having the structure:

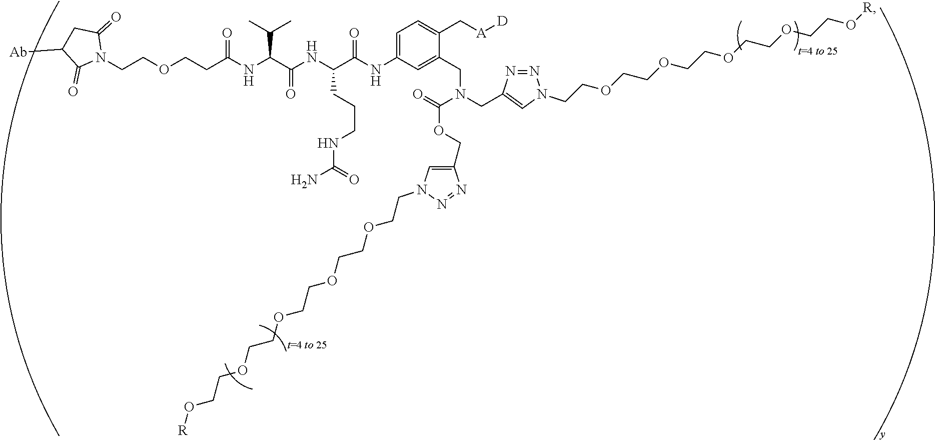

where each R is independently selected from H, —CH₃ or —CH₂CH₂C(═O)OH and y is 1, 2, 3, 4, 5, 6, 7, 8, 9, 10, 11, 12, 13, 14, 15 or 16.

Embodiment 78. The immunoconjugate of Formula (E′) or any one of Embodiments 62 to 72 having the structure:

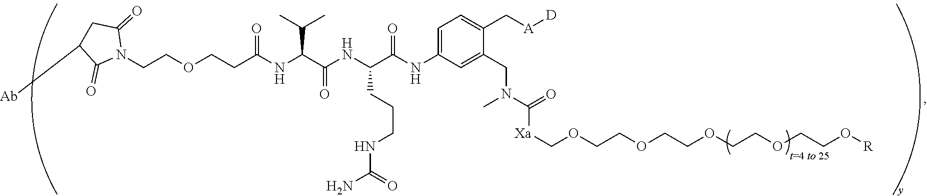

where Xa is —CH₂—, —OCH₂—, —NHCH₂— or —NRCH₂— and each R is independently H, —CH₃ or —CH₂CH₂C(═O)OH and y is 1, 2, 3, 4, 5, 6, 7, 8, 9, 10, 11, 12, 13, 14, 15 or 16.

Embodiment 79. The immunoconjugate of Formula (E′) or any one of Embodiments 62 to 72 having the structure:

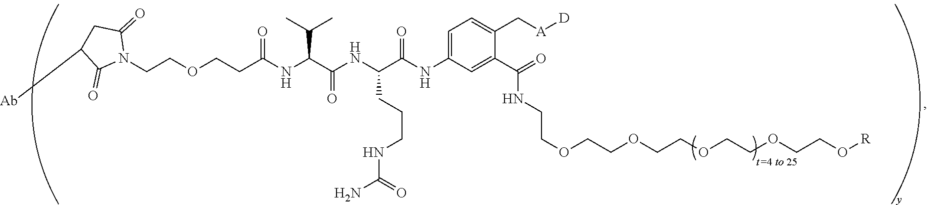

where R is H, —CH₃ or —CH₂CH₂C(═O)OH and y is 1, 2, 3, 4, 5, 6, 7, 8, 9, 10, 11, 12, 13, 14, 15 or 16.

Embodiment 80. The immunoconjugate of Formula (E′) or any one of Embodiments 62 to 72 having the structure:

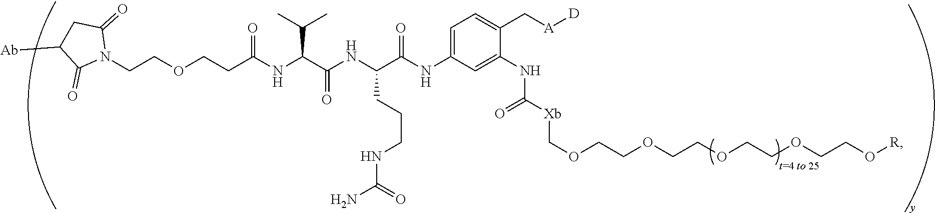

where Xb is —CH₂—, —OCH₂—, —NHCH₂— or —NRCH₂— and each R independently is H, —CH₃ or —CH₂CH₂C(═O)OH and y is 1, 2, 3, 4, 5, 6, 7, 8, 9, 10, 11, 12, 13, 14, 15 or 16.

Embodiment 81. The immunoconjugate of Formula (E′) or any one of Embodiments 62 to 72 having the structure:

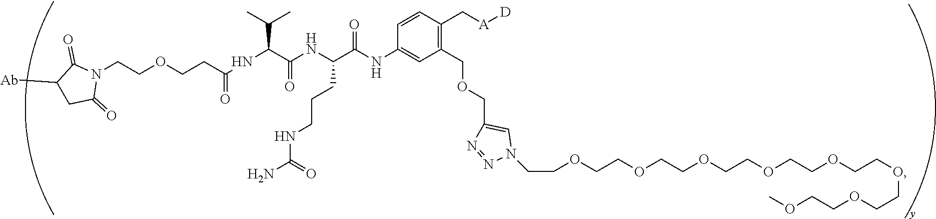

where y is 1, 2, 3, 4, 5, 6, 7, 8, 9, 10, 11, 12, 13, 14, 15 or 16.

Embodiment 82. The immunoconjugate of Formula (E′) or any one of Embodiments 62 to 72 having the structure:

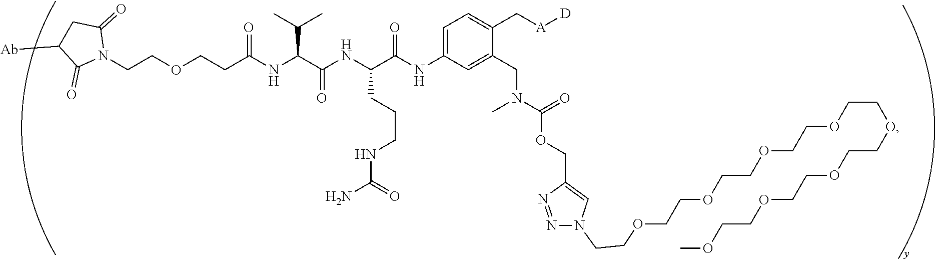

where y is 1, 2, 3, 4 , 5, 6, 7, 8,9, 10, 11, 12, 13, 14, 15 or 16.

Embodiment 83. The immunoconjugate of Formula (E′) or any one of Embodiments 62 to 72 having the structure:

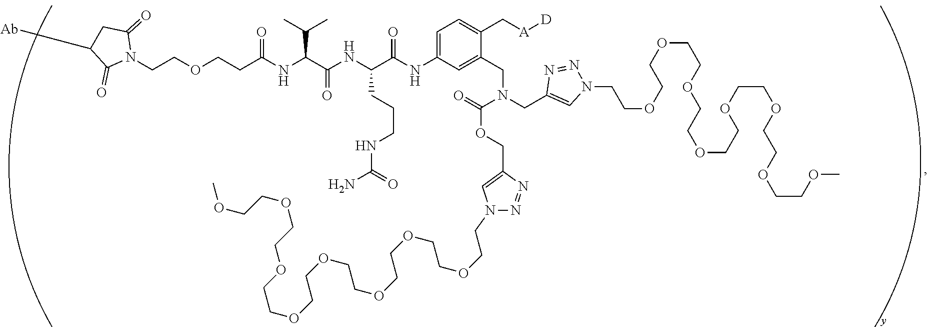

where y is 1, 2, 3, 4, 5, 6, 7, 8, 9, 10, 11, 12, 13, 14, 15 or 16.

Embodiment 84. The immunoconjugate of Formula (E′) or any one of Embodiments 62 to 72 having the structure:

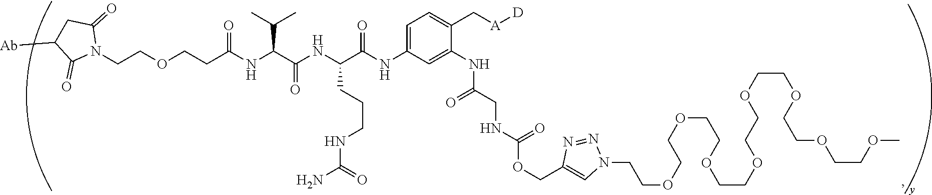

where y is 1, 2, 3, 4, 5, 6, 7, 8, 9, 10, 11, 12, 13, 14, 15 or 16.

Embodiment 85. The immunoconjugate of Formula (E′) or any one of Embodiments 62 to 72 having the structure:

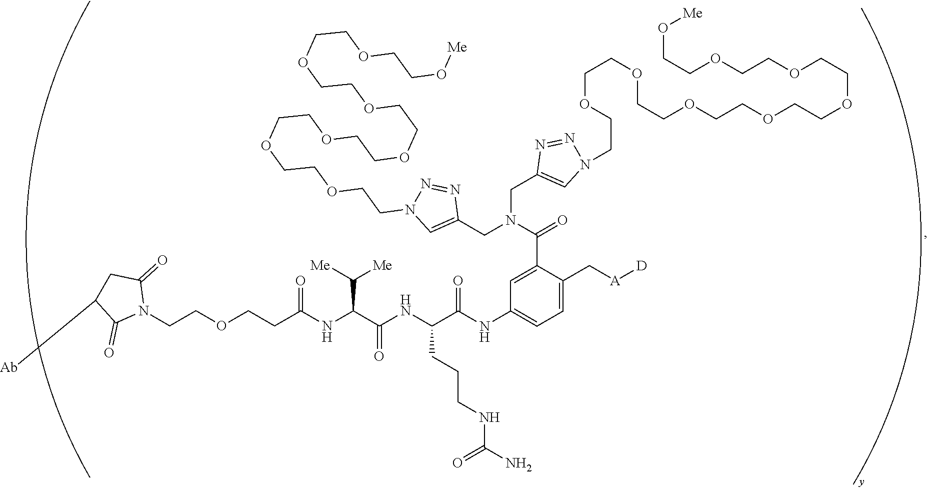

where y is 1, 2, 3, 4, 5, 6, 7, 8, 9, 10, 11, 12, 13, 14, 15 or 16

Embodiment 86. The immunoconjugate of Formula (E′) or any one of Embodiments 62 to 72 having the structure:

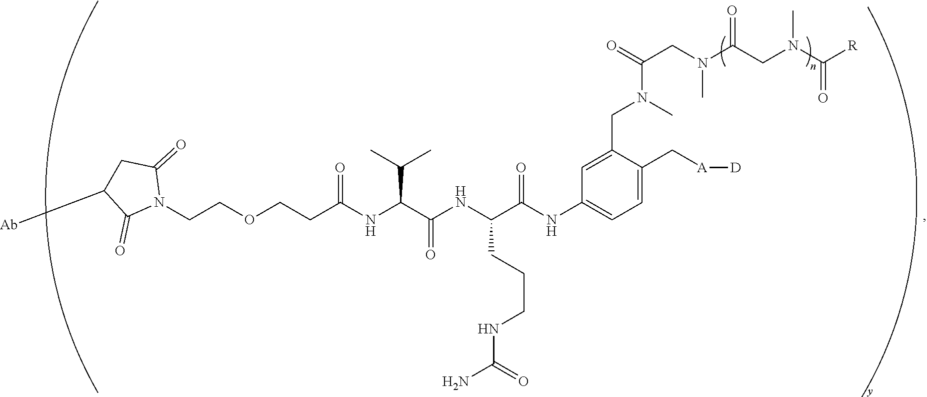

where y is 1, 2, 3, 4, 5, 6, 7, 8, 9, 10, 11, 12, 13, 14, 15 or 16.

Certain aspects and examples of the Linker-Drug groups, the conjugate linkers and the Antibody Drug Conjugates of the invention are provided in the following listing of additional enumerated embodiments. It will be recognized that features specified in each embodiment may be combined with other specified features to provide further embodiments of the present invention.

Embodiment 87. The compound of Formula (A′) or any one of Embodiments 1 to 2, or pharmaceutically acceptable salt thereof, the conjugate linker of Formula (C′) or any one of Embodiments 33 to 40, and the immunoconjugate of Formula (E′) or any one of Embodiments 62 to 63, wherein:
G is

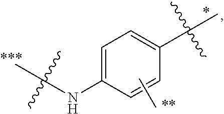

where the * of G indicates the point of attachment to L₂, and the ** of G indicates the point of attachment to L₃ and the *** of G indicates the point of attachment to Lp.

Embodiment 88. The compound of Formula (A′) or any one of Embodiments 1 to 2, or pharmaceutically acceptable salt thereof, the conjugate linker of Formula (C′) or any one of Embodiments 33 to 40, and the immunoconjugate of Formula (E′) or any one of Embodiments 62 to 63, wherein:
G is

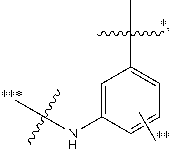

where the * of G indicates the point of attachment to L₂, and the ** of G indicates the point of attachment to L₃ and the *** of G indicates the point of attachment to Lp.

Embodiment 89. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, wherein:
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; *—C(═O)NH((CH₂)ₘO)ₜ(CH₂)ₙ—**; *—C(═O)O(CH₂)ₘSSC(R³)₂(CH₂)ₘC(═O)NR³(CH₂)ₘNR³C(═O)(CH₂)ₘ—**; *—C(═O)O(CH₂)ₘC(═O)NH(CH₂)ₘ—**; *—C(═O)(CH₂)ₘNH(CH₂)ₘ—**; *—C(═O)(CH₂)ₘNH(CH₂)ₙC(═O)—**; *—C(═O)(CH₂)ₘX(CH₂)ₘ—**; *—C(═O)((CH₂)ₘO)ₜ(CH₂)ₙX₁(CH₂)ₙ—**; *—C(═O)(CH₂)ₘNHC(═O)(CH₂)ₙ—**; *—C(═O)((CH₂)ₘO)ₜ(CH₂)ₙNHC(═O)(CH₂)ₙ—**; *—C(═O)(CH₂)ₘNHC(═O)(CH₂)ₙX₁(CH₂)ₙ—**; *—C(═O)((CH₂)ₘO)ₜ(CH₂)ₙNHC(═O)(CH₂)ₙX₁(CH₂)ₙ—**; *—C(═O)((CH₂)ₘO)ₜ(CH₂)ₙC(═O)NH(CH₂)ₘ—**; *—C(═O)(CH₂)ₘC(R³)₂—** or *—C(═O)(CH₂)ₘC(═O)NH(CH₂)ₘ—**, where the * of L₁ indicates the point of attachment to Lp, and the ** of L₁ indicates the point of attachment to R¹ if present or the ** of L₁ indicates the point of attachment to R¹⁰⁰ if present.

Embodiment 90. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, wherein:
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; *—C(═O)NH((CH₂)ₘO)ₜ(CH₂)ₙ—**; *—C(═O)(CH₂)ₘNH(CH₂)ₘ—**; *—C(═O)(CH₂)ₘNH(CH₂)ₙC(═O)—**; *—C(═O)(CH₂)ₘNHC(═O)(CH₂)ₙ—**; *—C(═O)((CH₂)ₘO)ₜ(CH₂)ₙNHC(═O)(CH₂)ₙ—**; *—C(═O)((CH₂)ₘO)ₜ(CH₂)ₙC(═O)NH(CH₂)ₘ—**; *—C(═O)(CH₂)ₘC(R³)₂—** or *—C(═O)(CH₂)ₘC(═O)NH(CH₂)ₘ—**, where the * of L₁ indicates the point of attachment to Lp, and the ** of L₁ indicates the point of attachment to R¹ if present or the ** of L₁ indicates the point of attachment to R¹⁰⁰ if present.

Embodiment 91. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, wherein:
L₁ is *—C(═O)(CH₂)ₘO(CH₂)ₘ—**; *—C(═O)((CH₂)ₘO)ₜ(CH₂)ₙ—**; *—C(═O)(CH₂)ₘ—**; *—C(═O)NH((CH₂)ₘO)ₜ(CH₂)ₙ—**; *—C(═O)(CH₂)ₘNH(CH₂)ₘ—**; *—C(═O)(CH₂)ₘNH(CH₂)ₙC(═O)—**; or *—C(═O)(CH₂)ₘNHC(═O)(CH₂)ₙ—**, where the * of L₁ indicates the point of attachment to Lp, and the ** of L₁ indicates the point of attachment to R¹ if present or the ** of L₁ indicates the point of attachment to R¹⁰⁰ if present.

Embodiment 92. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, wherein:
L₁ is *—C(═O)(CH₂)ₘO(CH₂)ₘ—**; *—C(═O)((CH₂)ₘO)ₜ(CH₂)ₙ—**; *—C(═O)(CH₂)ₘ—** or *—C(═O)NH((CH₂)ₘO)ₜ(CH₂)ₙ—**, where the * of L₁ indicates the point of attachment to Lp, and the ** of L₁ indicates the point of attachment to R¹ if present or the ** of L₁ indicates the point of attachment to R¹⁰⁰ if present.

Embodiment 93. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, wherein L₁ is *—C(═O)(CH₂)ₘO(CH₂)ₘ—**, where the * of L₁ indicates the point of attachment to Lp, and the ** of L₁ indicates the point of attachment to R¹ if present or the ** of L₁ indicates the point of attachment to R¹⁰⁰ if present.

Embodiment 94. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, wherein L₁ is *—C(═O)((CH₂)ₘO)ₜ(CH₂)ₙ—**, where the * of L₁ indicates the point of attachment to Lp, and the ** of L₁ indicates the point of attachment to R¹ if present or the ** of L₁ indicates the point of attachment to R¹⁰⁰ if present.

Embodiment 95. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, wherein L₁ is *—C(═O)(CH₂)ₘ—**, where the * of L₁ indicates the point of attachment to Lp, and the ** of L₁ indicates the point of attachment to R¹ if present or the ** of L₁ indicates the point of attachment to R¹⁰⁰ if present.

Embodiment 96. The compound of Formula (A′) or any one of Embodiments 1 to 17, or pharmaceutically acceptable salt thereof, the conjugate linker of Formula (C′) or any one of Embodiments 32 to 46, and the immunoconjugate of Formula (E′) or any one of Embodiments 60 to 70, wherein L₁ is *—C(═O)NH((CH₂)ₘO)ₜ(CH₂)ₙ—**, where the * of L₁ indicates the point of attachment to Lp, and the ** of L₁ indicates the point of attachment to R¹ if present or the ** of L₁ indicates the point of attachment to R¹⁰⁰ if present.

Embodiment 97. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 84 to 93, wherein Lp is an enzymatically cleavable bivalent peptide spacer.

Embodiment 98. The compound of Formula (A′) or any one of Embodiments 1 to 17, or pharmaceutically acceptable salt thereof, the conjugate linker of Formula (C′) or any one of Embodiments 32 to 46, and the immunoconjugate of Formula (E′) or any one of Embodiments 60 to 70, or any one of Embodiments 87 to 97, wherein Lp is a bivalent peptide spacer comprising an amino acid residue selected from glycine, valine, citrulline, lysine, isoleucine, phenylalanine, methionine, asparagine, proline, alanine, leucine, tryptophan, and tyrosine.

Embodiment 99. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 98, wherein Lp is a bivalent peptide spacer comprising two to four amino acid residues.

Embodiment 100. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 99, wherein Lp is a bivalent peptide spacer comprising two to four amino acid residues each independently selected from glycine, valine, citrulline, lysine, isoleucine, phenylalanine, methionine, asparagine, proline, alanine, leucine, tryptophan, and tyrosine.

Embodiment 101. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 100, wherein:
Lp is a bivalent peptide spacer selected from

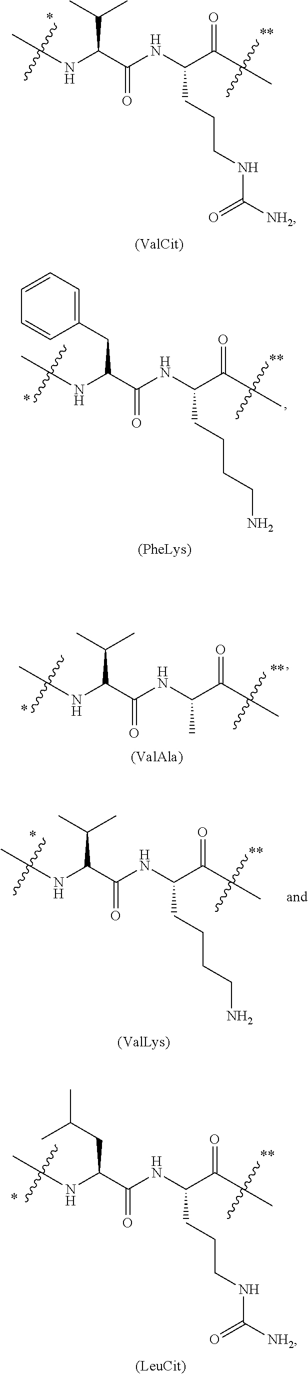

where the * of Lp indicates the attachment point to L₁ and the ** of Lp indicates the attachment point to the —NH— group of Formula (B′) or the ** of Lp indicates the attachment point to the G of Formula (A′).

Embodiment 102. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 101, wherein:
Lp is

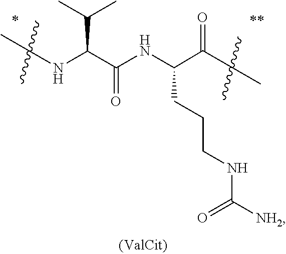 [SMILES: *N[C@H](C(=O)N[C@@H](CCCNC(N)=O)C(*)=O)C(C)C]

where the * of Lp indicates the attachment point to L₁ and the ** of Lp indicates the attachment point to the —NH— group of Formula (B′) or the ** of Lp indicates the attachment point to the G of Formula (A′).

Embodiment 103. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 101, wherein:
Lp is

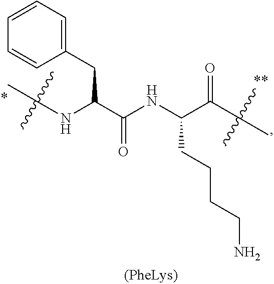

where the * of Lp indicates the attachment point to L₁ and the ** of Lp indicates the attachment point to the —NH— group of Formula (B′) or the ** of Lp indicates the attachment point to the G of Formula (A′).

Embodiment 104. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 101, wherein:
Lp is

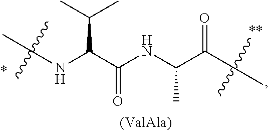

where the * of Lp indicates the attachment point to L₁ and the ** of Lp indicates the attachment point to the —NH— group of Formula (B′) or the ** of Lp indicates the attachment point to the G of Formula (A′).

Embodiment 105. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 101, wherein:
Lp is

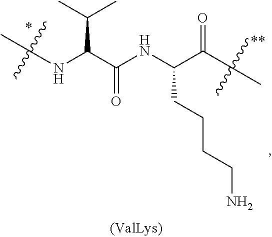

where the * of Lp indicates the attachment point to L₁ and the ** of Lp indicates the attachment point to the —NH— group of Formula (B′) or the ** of Lp indicates the attachment point to the G of Formula (A′).

Embodiment 106. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 101, wherein:
Lp is

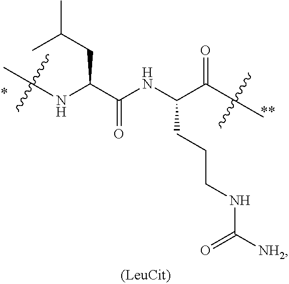 [SMILES: *N[C@@H](CC(C)C)C(=O)N[C@@H](CCCNC(N)=O)C(*)=O]

where the * of Lp indicates the attachment point to L₁ and the ** of Lp indicates the attachment point to —NH— group of Formula (B′) or the ** of Lp indicates the attachment point to the G of Formula (A′).

Embodiment 107. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 106, wherein L₂ is a bond, a methylene, or a C₂-C₃alkenylene.

Embodiment 108. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 107, wherein L₂ is a bond or a methylene.

Embodiment 109. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 108, wherein L₂ is a bond.

Embodiment 110. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 108, wherein L₂ is a methylene.

Embodiment 111. The compound of Formula (A′) or any one of Embodiments 1 to 30, or pharmaceutically acceptable salt thereof, the conjugate linker of Formula (C′) or any one of Embodiments 33 to 47, and the immunoconjugate of Formula (E′) or any one of Embodiments 62 to 85, or any one of Embodiments 87 to 110, wherein:
A is a bond, —OC(═O)—, —OC(═O)N(CH₃)CH₂CH₂N(CH₃)C(═O)— or —OC(═O)N(CH₃)C(R^{a})₂C(R^{a})₂N(CH₃)C(═O)—, wherein each R^{a} is independently selected from H, C₁-C₆alkyl or a C₃-C₈acycloalkyl.

Embodiment 112. The compound of Formula (A′) or any one of Embodiments 1 to 32, or pharmaceutically acceptable salt thereof, the conjugate linker of Formula (C′) or any one of Embodiments 33 to 61, and the immunoconjugate of Formula (E′) or any one of Embodiments 62 to 86, or any one of Embodiments 87 to 111, wherein A is a bond or —OC(═O).

Embodiment 113. The compound of Formula (A′) or any one of Embodiments 1 to 32, or pharmaceutically acceptable salt thereof, the conjugate linker of Formula (C′) or any one of Embodiments 33 to 61, and the immunoconjugate of Formula (E′) or any one of Embodiments 62 to 86, or any one of Embodiments 87 to 112, wherein A is a bond.

Embodiment 114. The compound of Formula (A′) or any one of Embodiments 1 to 32, or pharmaceutically acceptable salt thereof, the conjugate linker of Formula (C′) or any one of Embodiments 33 to 61, and the immunoconjugate of Formula (E′) or any one of Embodiments 62 to 86, or any one of Embodiments 87 to 112, wherein A is —OC(═O).

Embodiment 115. The compound of Formula (A′) or any one of Embodiments 1 to 32, or pharmaceutically acceptable salt thereof, the conjugate linker of Formula (C′) or any one of Embodiments 33 to 61, and the immunoconjugate of Formula (E′) or any one of Embodiments 62 to 86, or any one of Embodiments 87 to 110, wherein:
A is

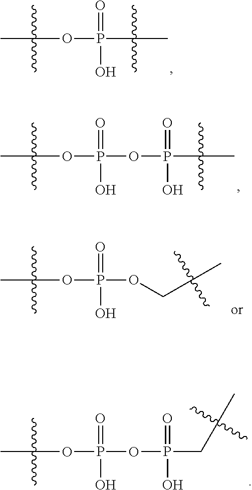 [SMILES: *OP(*)(=O)O ; *OP(=O)(O)OP(*)(=O)O ; *COP(=O)(O)O* ; *CP(=O)(O)OP(=O)(O)O*]

Embodiment 116. The compound of Formula (A′) or any one of Embodiments 1 to 32, or pharmaceutically acceptable salt thereof, the conjugate linker of Formula (C′) or any one of Embodiments 33 to 61, and the immunoconjugate of Formula (E′) or any one of Embodiments 62 to 85, or any one of Embodiments 86 to 110, wherein:
A is —OC(═O)N(CH₃)CH₂CH₂N(CH₃)C(═O)— or —OC(═O)N(CH₃)C(R^{a})₂C(R^{a})₂N(CH₃)C(═O)—,
wherein each R^{a} is independently selected from H, C₁-C₆alkyl or a C₃-C₈cycloalkyl.

Embodiment 117. The compound of Formula (A′) or any one of Embodiments 1 to 17, or pharmaceutically acceptable salt thereof, the conjugate linker of Formula (C′) or any one of Embodiments 33 to 49, and the immunoconjugate of Formula (E′) or any one of Embodiments 62 to 72, or any one of Embodiments 87 to 116, wherein:
L₃ is a spacer moiety having the structure

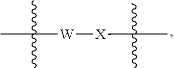

where
   W is —CH₂O—**, —CH₂N(R^{b})C(═O)O—**, —NHC(═O)C(R^{b})₂NHC(═O)O—**, —NHC(═O)C(R^{b})₂NH—**, —NHC(═O)C(R^{b})₂NHC(═O)—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H C₁-C₆alkyl or C₃-C₈acycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²;
   and
the * of L₃ indicates the point of attachment to R².

Embodiment 118. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 117, wherein:
L₃ is a spacer moiety having the structure

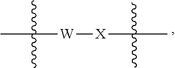

where
   W is —CH₂O—**, —CH₂N(R^{b})C(═O)O—**, —NHC(═O)CH₂NHC(═O)O—**, —NHC(═O)CH₂NH—**, —NHC(═O)CH₂NHC(═O)—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H C₁-C₆alkyl or C₃-C₈cycloalkyl and wherein the ** of W indicates the point of attachment to X;
   X is a bond;
   and
the * of L₃ indicates the point of attachment to R².

Embodiment 119. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 118, wherein:
L₃ is a spacer moiety having the structure

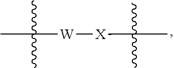

where
   W is —CH₂O—**, —CH₂N(R^{b})C(═O)O—**, —NHC(═O)CH₂NHC(═O)O—**, —NHC(═O)CH₂NH—**, —NHC(═O)CH₂NHC(═O)—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H C₁-C₆alkyl or C₃-C₈cycloalkyl and wherein the ** of W indicates the point of attachment to X;
   X is a triazolyl, wherein the *** of X indicates the point of attachment to W and the * of X indicates the point of attachment to R²
   and
the * of L₃ indicates the point of attachment to R².

Embodiment 120. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 118, wherein:
L₃ is a spacer moiety having the structure

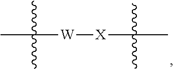

where
   W is —CH₂O—**, —CH₂N(R^{b})C(═O)O—**, —NHC(═O)CH₂NHC(═O)O—**, —NHC(═O)CH₂NH—**, —NHC(═O)CH₂NHC(═O)—**, —CH₂N(X—R²)C(═O)O—**, —C(═O)N(X—R²)—**, —CH₂N(X—R²)C(═O)—**, —C(═O)NR^{b}—**, —C(═O)NH—**, —CH₂NR^{b}C(═O)—**, —CH₂NR^{b}C(═O)NH—**, —CH₂NR^{b}C(═O)NR^{b}—**, —NHC(═O)—**, —NHC(═O)O—**, —NHC(═O)NH—**, —OC(═O)NH—**, —S(O)₂NH—**, —NHS(O)₂—**, —C(═O)—, —C(═O)O—** or —NH—, wherein each R^{b} is independently selected from H, C₁-C₆alkyl or C₃-C₈cycloalkyl and wherein the ** of W indicates the point of attachment to X;
   X is ***—CH₂-triazolyl-*, wherein the *** of X indicates the point of attachment to W and the * of X indicates the point of attachment to R²
   and
the * of L₃ indicates the point of attachment to R².

Embodiment 121. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 118, wherein:
L₃ is a spacer moiety having the structure

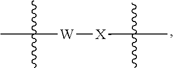

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, wherein each R^{b} is independently selected from H, C₁-C₆alkyl or C₃-C₈acycloalkyl and wherein the ** of W indicates the point of attachment to X;
   X is a bond, triazolyl or ***—CH₂-triazolyl-*, wherein the *** of X indicates the point of attachment to W and the * of X indicates the point of attachment to R²
   and
the * of L₃ indicates the point of attachment to R².

Embodiment 122. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 118, wherein:
L₃ is a spacer moiety having the structure

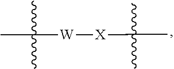

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, wherein each R^{b} is independently selected from H, C₁-C₆alkyl or C₃-C₈cycloalkyl and wherein the ** of W indicates the point of attachment to X;
   X is a bond;
   and
the * of L₃ indicates the point of attachment to R².

Embodiment 123. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 83 to 118, wherein:
L₃ is a spacer moiety having the structure

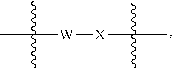

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, wherein each R^{b} is independently selected from H, C₁-C₆alkyl or C₃-C₈cycloalkyl and wherein the ** of W indicates the point of attachment to X;
   X is a triazolyl, wherein the *** of X indicates the point of attachment to W and the * of X indicates the point of attachment to R²;
   and
the * of L₃ indicates the point of attachment to R².

Embodiment 124. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 118, wherein:
L₃ is a spacer moiety having the structure

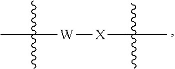

where
   W is —CH₂O—**, —CH₂N(R^{b})C(═O)O—**, —NHC(═O)CH₂NHC(═O)O—**, —CH₂N(X—R²)C(═O)O—**, —C(═O)N(X—R²)—**, wherein each R^{b} is independently selected from H, C₁-C₆alkyl or C₃-C₈cycloalkyl and wherein the ** of W indicates the point of attachment to X;
   X is ***—CH₂-triazolyl-*, wherein the *** of X indicates the point of attachment to W and the * of X indicates the point of attachment to R²
   and
the * of L₃ indicates the point of attachment to R².

Embodiment 125. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 86 to 124, wherein R² is a hydrophilic moiety selected from polyethylene glycol, polyalkylene glycol, a sugar, an oligosaccharide, a polypeptide or C₂-C₆alkyl substituted with 1 to 3

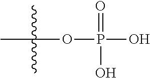 [SMILES: *OP(=O)(O)O]

groups.

Embodiment 126. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 125, wherein R² is a sugar.

Embodiment 127. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 125, wherein R² is an oligosaccharide.

Embodiment 128. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 125, wherein R² is a polypeptide.

Embodiment 129. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 125, wherein R² is a polyalkylene glycol.

Embodiment 130. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 125, wherein R² is a polyalkylene glycol having the structure —(O(CH₂)ₘ)ₜR′, where R′ is OH, OCH₃ or OCH₂CH₂C(═O)OH, m is 1-10 and t is 4-40.

Embodiment 131. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 125, wherein R² is a polyalkylene glycol having the structure —((CH₂)ₘO)ₜR″—, where R″ is H, CH₃ or CH₂CH₂C(═O)OH, m is 1-10 and t is 4-40.

Embodiment 132. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 125, wherein R² is a polyethylene glycol.

Embodiment 133. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 125, wherein R² is a polyethylene glycol having the structure —(OCH₂CH₂)ₜR′, where R′ is OH, OCH₃ or OCH₂CH₂C(═O)OH and t is 4-40,

Embodiment 134. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 125, wherein R² is a polyethylene glycol having the structure —(CH₂CH₂O)ₜR″—, where R″ is H, CH₃ or CH₂CH₂C(═O)OH and t is 4-40.

Embodiment 135. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 125, wherein:
R² is

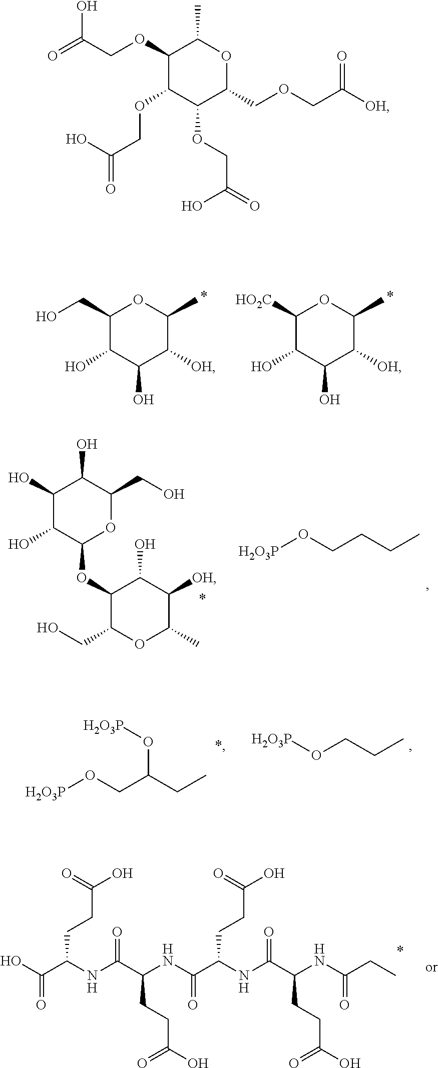

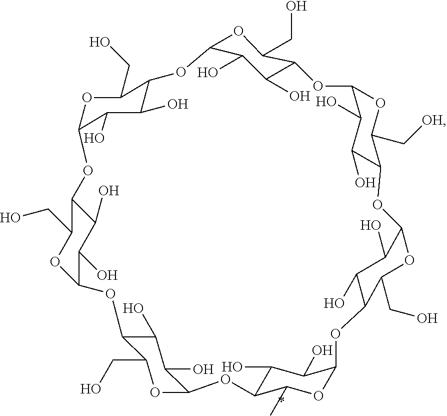

where the * of R² indicates the point of attachment to X or L₃.

Embodiment 136. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 125, wherein:

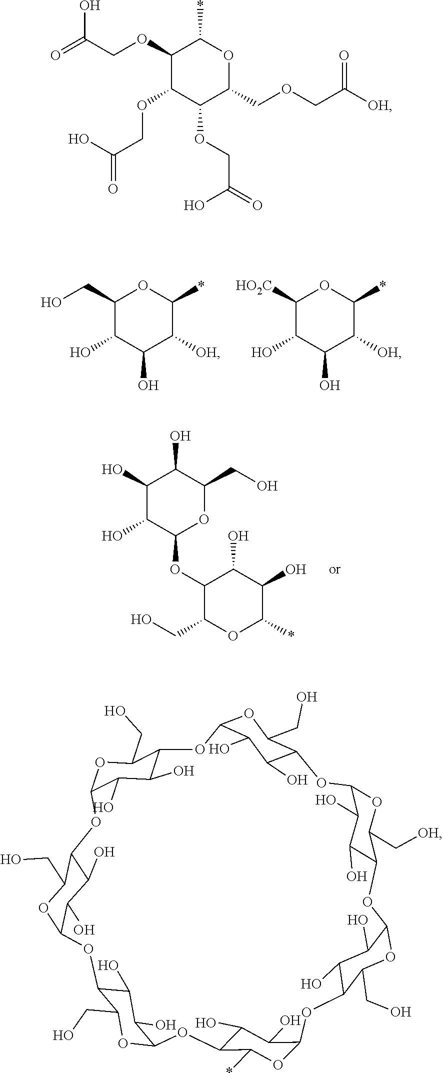 [SMILES: *[C@@H]1O[C@H](COCC(=O)O)[C@H](OCC(=O)O)[C@H](OCC(=O)O)[C@H]1OCC(=O)O ; *[C@@H]1O[C@H](CO)[C@@H](O)[C@H](O)[C@H]1O ; *[C@@H]1O[C@H](C(=O)O)[C@@H](O)[C@H](O)[C@H]1O ; *[C@@H]1O[C@H](CO)C(O[C@@H]2O[C@H](CO)[C@H](O)[C@H](O)[C@H]2O)[C@H](O)[C@H]1O ; *C1OC2OC3C(CO)OC(OC4C(CO)OC(OC5C(CO)OC(OC6C(CO)OC(OC7C(CO)OC(OC8C(CO)OC(OC1C(O)C2O)C(O)C8O)C(O)C7O)C(O)C6O)C(O)C5O)C(O)C4O)C(O)C3O]

where the * of R² indicates the point of attachment to X or L₃.

Embodiment 137. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 125, wherein:
R² is

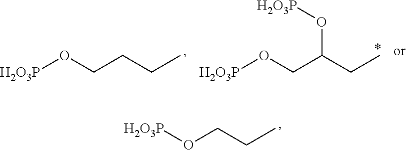

where the * of R² indicates the point of attachment to X or L₃.

Embodiment 138. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 125, wherein:

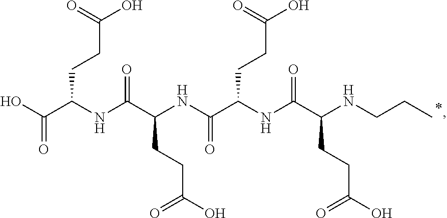

R² is
where the * of R² indicates the point of attachment to X or L₃.

Embodiment 139. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 138, wherein:
X₁ is

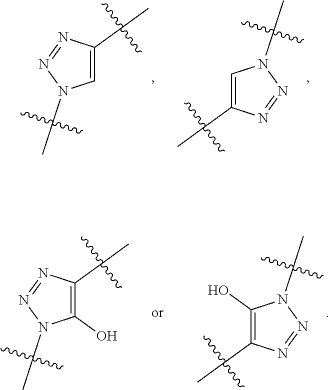 [SMILES: *c1cn(*)nn1 ; *c1cn(*)nn1 ; *c1nnn(*)c1O ; *c1nnn(*)c1O]

Embodiment 140. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 138, wherein:
X₁ is

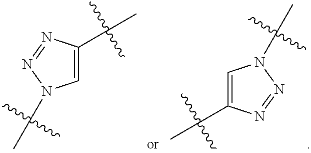 [SMILES: *c1cn(*)nn1 ; *c1cn(*)nn1]

Embodiment 141. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 140, wherein:
each m is independently selected from 1, 2, 3, 4, and 5.

Embodiment 142. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 140, wherein:
each m is independently selected from 1, 2 and 3.

Embodiment 143. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 142, wherein:
each n is independently selected from 1, 2, 3, 4 and 5.

Embodiment 144. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 142, wherein:
each n is independently selected from 1, 2 and 3.

Embodiment 145. The compound of Formula (A′) or any one of Embodiments 1 to 17, or pharmaceutically acceptable salt thereof, the linker of Formula (C′) or any one of Embodiments 33 to 47, and the immunoconjugate of Formula (E′) or any one of Embodiments 62 to 72, or any one of Embodiments 87 to 144, wherein:
each t is independently selected from 2, 3, 4, 5, 6, 7, 8, 9, 10, 11, 12, 13, 14, 15, 16, 17, 18, 19, 20, 21, 22, 23, 24, 25, 26, 27, 28, 29 and 30.

Embodiment 146. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 144, wherein:
each t is independently selected from 4, 5, 6, 7, 8, 9, 10, 11, 12, 13, 14, 15, 16, 17, 18, 19,20,21,22,23,24 or 25.

Embodiment 147. The compound of Formula (A′) or any one of Embodiments 1 to 17, or pharmaceutically acceptable salt thereof, the conjugate linker of Formula (C′) or any one of Embodiments 33 to 47, and the immunoconjugate of Formula (E′) or any one of Embodiments 62 to 72, or any one of Embodiments 87 to 144, wherein:
each t is independently selected from 1, 2, 3, 4, 5, 6, 7, 8, 9, 10, 11, 12, 13, 14, 15, 16, 17 and 18.

Embodiment 148. The immunoconjugate of Formula (E′) or any one of Embodiments 62 to 72, or any one of Embodiments 87 to 147, wherein y is 1, 2, 3, 4, 5, 6, 7, 8, 9, 10, 11, 12, 13, or 14.

Embodiment 149. The immunoconjugate of Formula (E′) or any one of Embodiments 62 to 72, or any one of Embodiments 87 to 147, wherein y is 1, 2, 3, 4, 5, 6, 7, 8, 9, 10, 11 or 12.

Embodiment 150. The immunoconjugate of Formula (E′) or any one of Embodiments 62 to 72, or any one of Embodiments 87 to 147, wherein y is 1, 2, 3, 4, 5, 6, 7, 8, 9 or 10.

Embodiment 151. The immunoconjugate of Formula (E′) or any one of Embodiments 62 to 72, or any one of Embodiments 87 to 147, wherein y is 1, 2, 3, 4, 5, 6, 7 or 8.

Embodiment 152. The immunoconjugate of Formula (E′) or any one of Embodiments 62 to 72, or any one of Embodiments 87 to 147, wherein y is 1, 2, 3, 4, 5 or 6.

Embodiment 153. The immunoconjugate of Formula (E′) or any one of Embodiments 62 to 72, or any one of Embodiments 87 to 147, wherein y is 1, 2, 3 or 4.

Embodiment 154. The immunoconjugate of Formula (E′) or any one of Embodiments 62 to 72, or any one of Embodiments 87 to 147, wherein y is 1 or 2.

Embodiment 155. The immunoconjugate of Formula (E′) or any one of Embodiments 62 to 72, or any one of Embodiments 87 to 147, wherein y is 2.

Embodiment 156. The immunoconjugate of Formula (E′) or any one of Embodiments 62 to 72, or any one of Embodiments 87 to 147, wherein y is 4.

Embodiment 157. The immunoconjugate of Formula (E′) or any one of Embodiments 62 to 72, or any one of Embodiments 87 to 147, wherein y is 6.

Embodiment 158. The immunoconjugate of Formula (E′) or any one of Embodiments 62 to 72, or any one of Embodiments 87 to 147, wherein y is 8.

Embodiment 159. The compound of Formula (A′) or any one of Embodiments 1 to 31, or pharmaceutically acceptable salt thereof, the immunoconjugate of Formula (E′) or any one of Embodiments 62 to 72, or any one of Embodiments 87 to 158, wherein D is a panRAS inhibitor when released from the immunoconjugates.

### Other Conjugate Linker Groups

Other examples of conjugate linker groups that are suitable for making ADCs or immunoconjugates of a panRAS inhibitor disclosed herein includes those disclosed in international application publications such as WO2018200812, WO2017214456, WO2017214458, WO2017214462, WO2017214233, WO2017214282, WO2017214301, WO2017214322, WO2017214335, WO2017214339, WO2016094509, WO2016094517, and WO2016094505, the contents of each of which are incorporated by reference in their entireties.

For example, the immunoconjugates of panRAS inhibitors disclosed herein can have a linker-payload (“-L-D”) structure selected from:

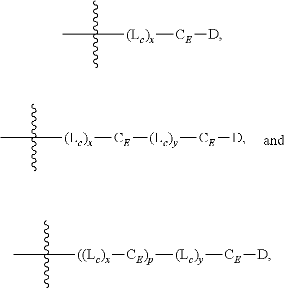

wherein:
Lc is a conjugate linker component and each Lc is independently selected from a conjugate linker component as disclosed herein;
x is an integer selected from 1, 2, 3, 4, 5, 6, 7, 8, 9, 10, 11, 12, 13, 14, 15, 16, 17, 18, 19 and 20;
y is an integer selected from 1, 2, 3, 4, 5, 6, 7, 8, 9, 10, 11, 12, 13, 14, 15, 16, 17, 18, 19 and 20;
p is an integer selected from 1, 2, 3, 4, 5, 6, 7, 8, 9 and 10;
D is a panRAS inhibitor disclosed herein;
and each cleavage element (C_{E}) is independently selected from a self-immolative spacer and a group that is susceptible to cleavage selected from acid-induced cleavage, peptidase-induced cleavage, esterase-induced cleavage, glycosidase induced cleavage, phosphodiesterase induced cleavage, phosphatase induced cleavage, protease induced cleavage, lipase induced cleavage or disulfide bond cleavage.

In some embodiments, L has a structure selected from the following, or L comprises a structural component selected from the following:

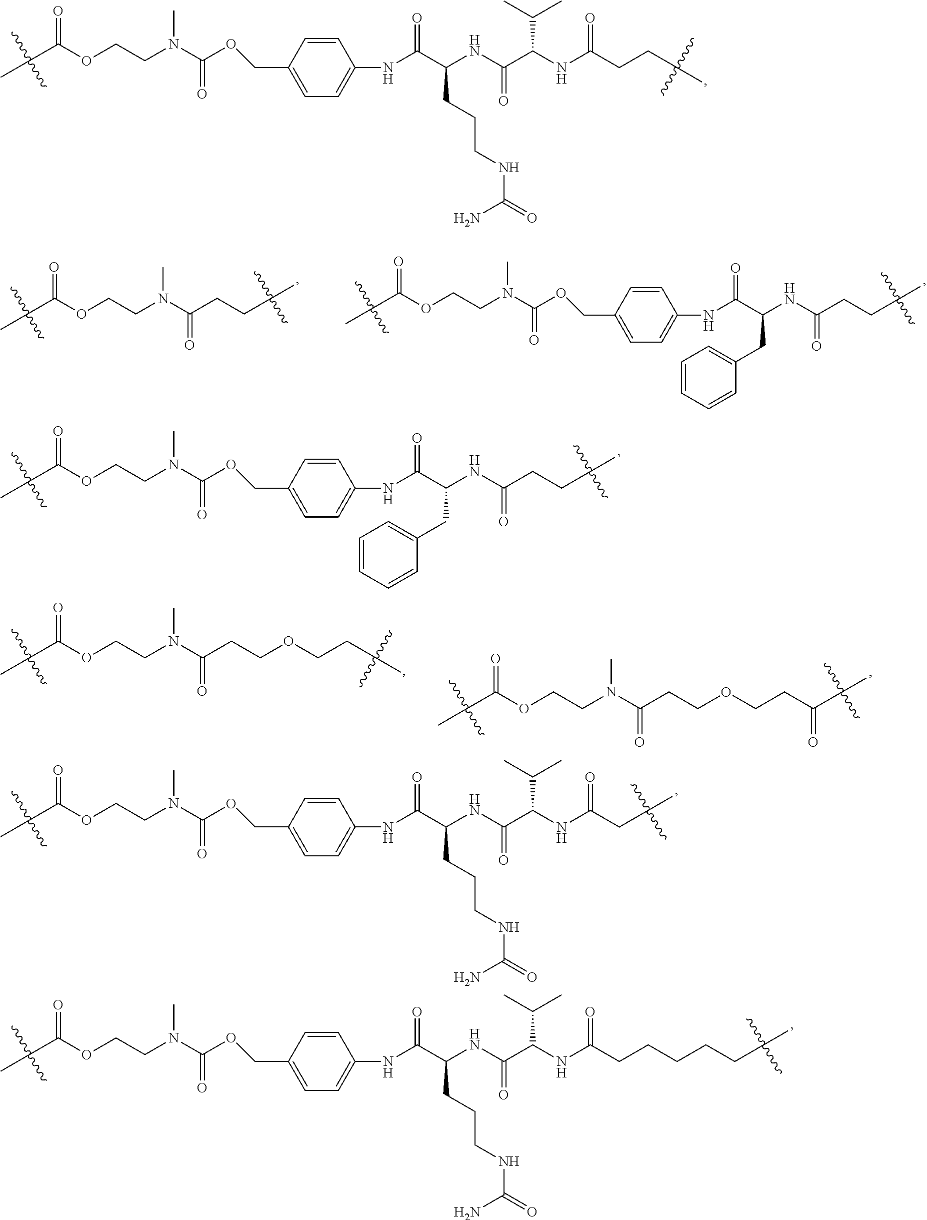

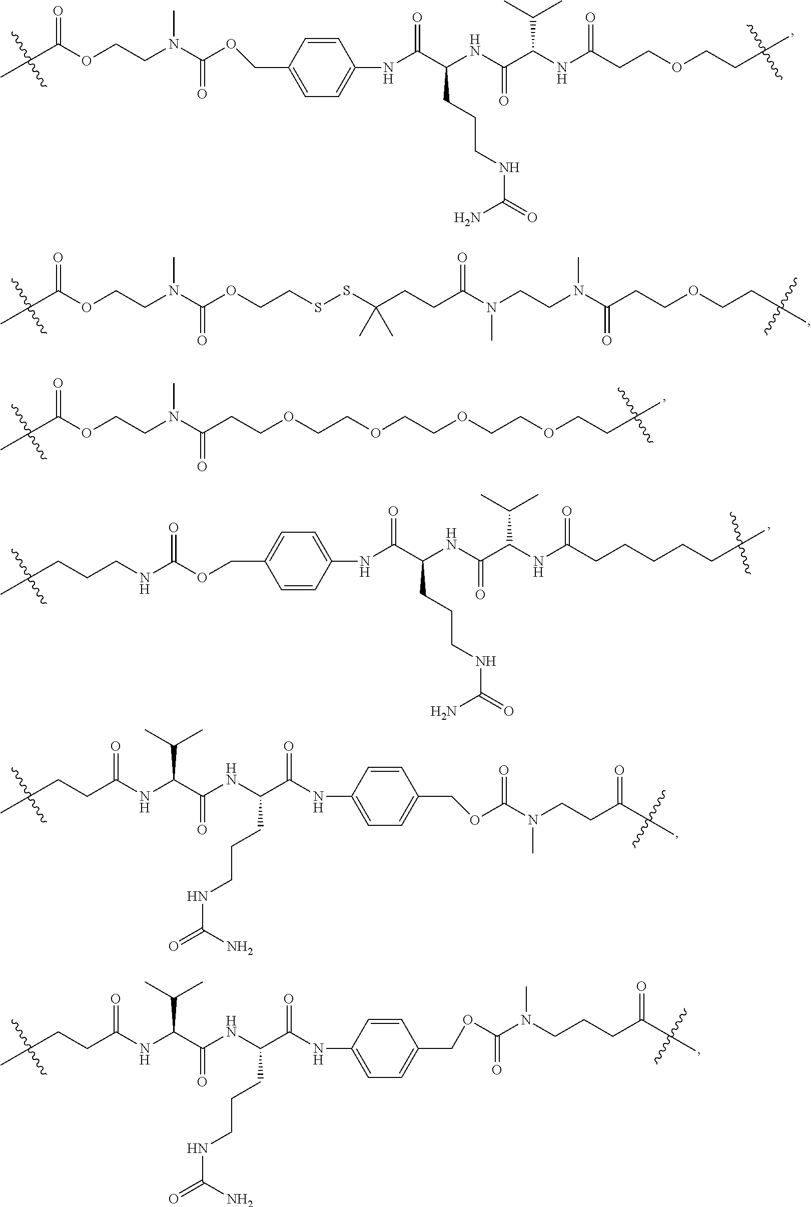

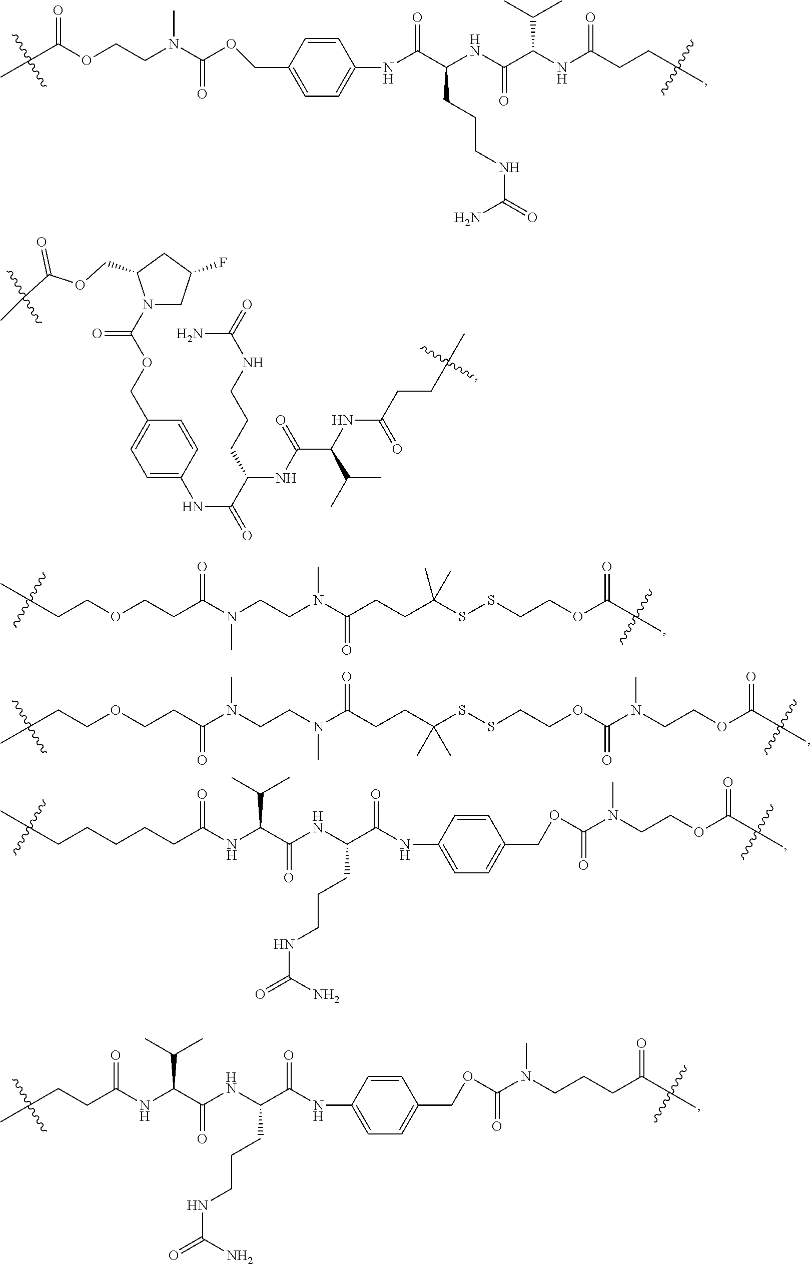

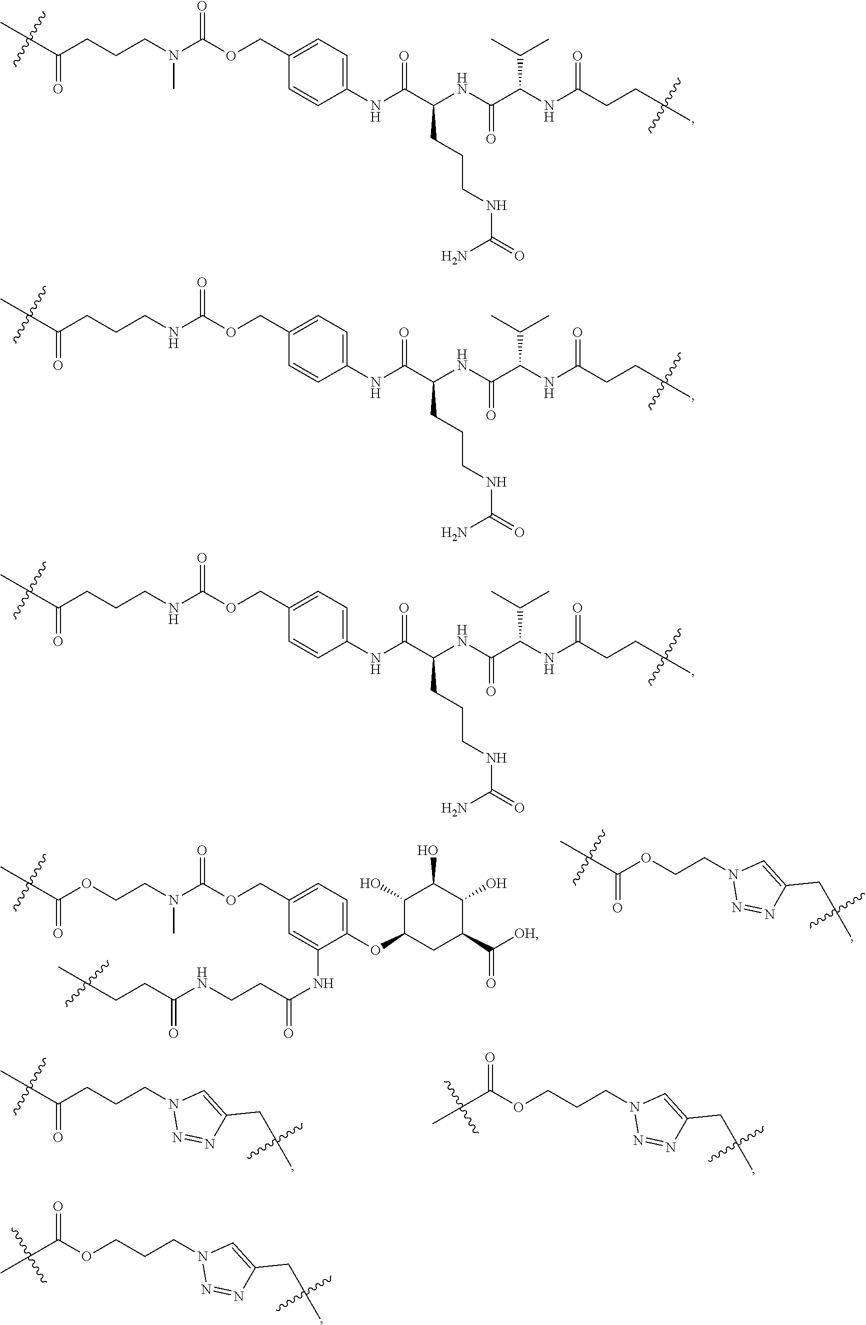

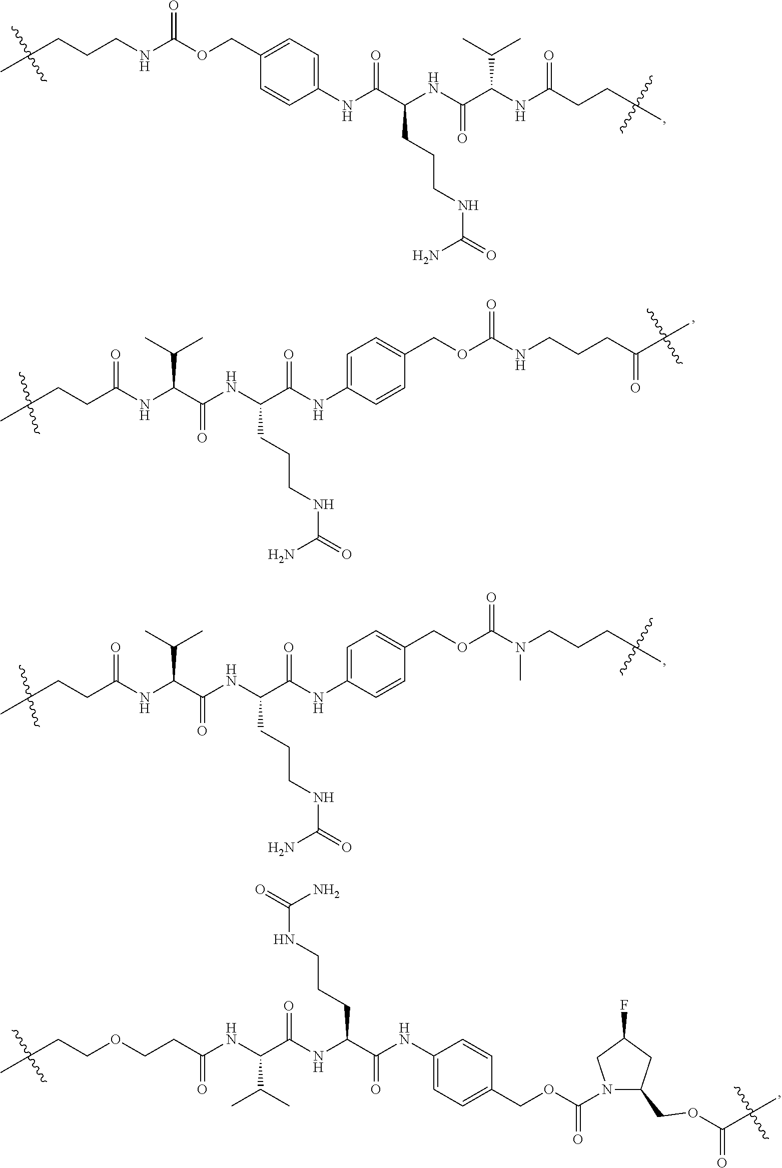

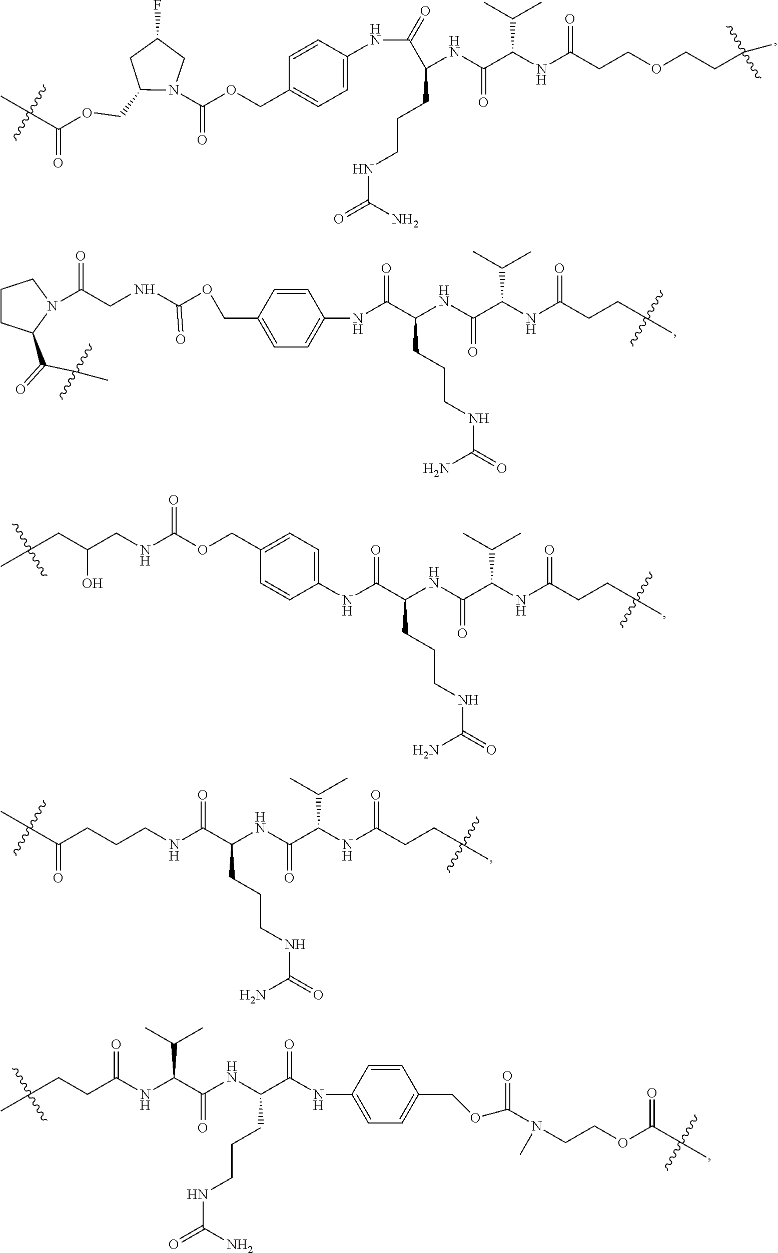

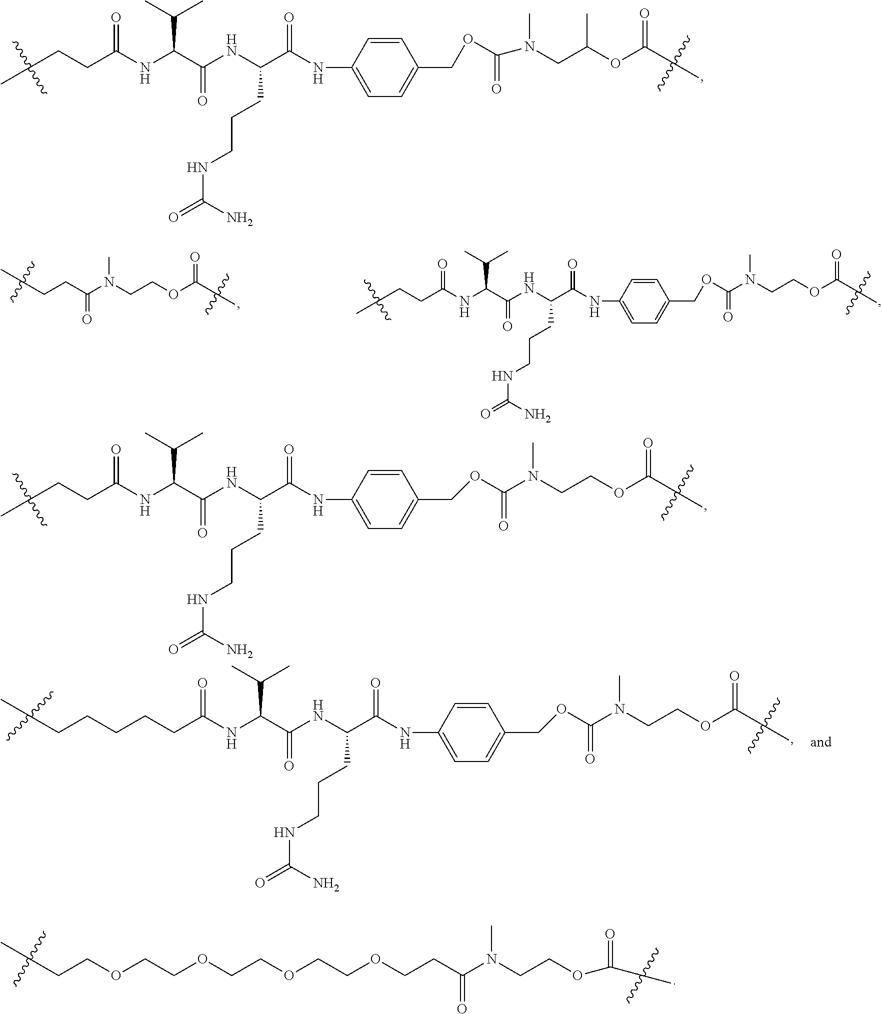

In some embodiments, Lc is a conjugate linker component and each Lc is independently selected from

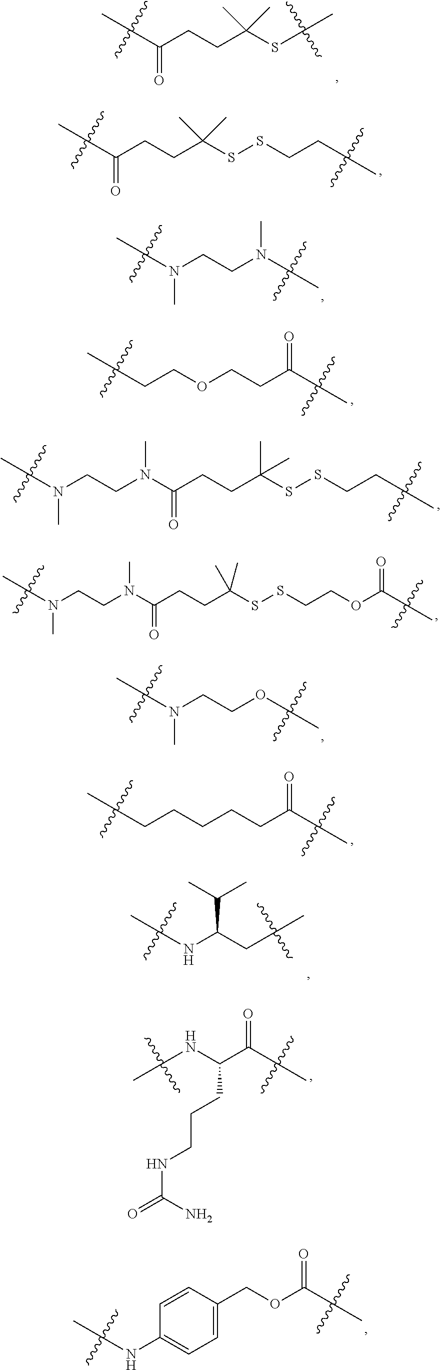

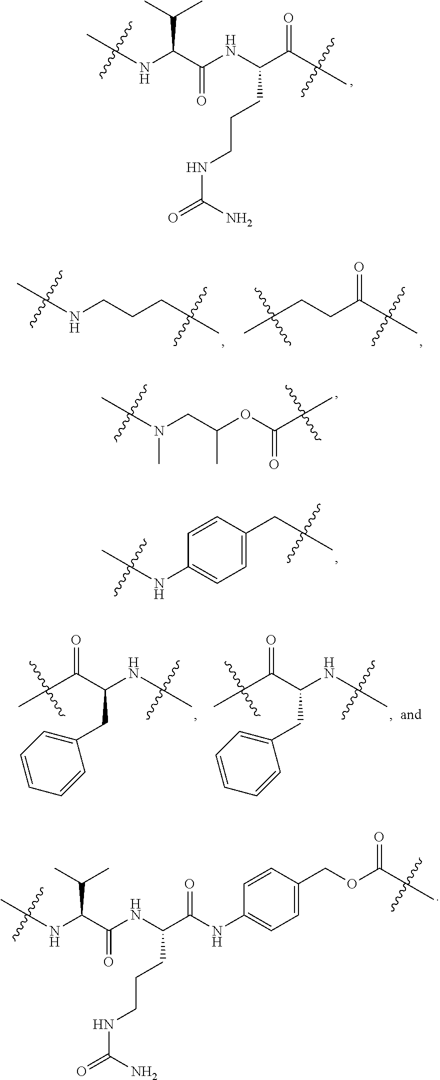

In some embodiments, the conjugate linker L comprises a conjugate linker component that is selected from:
—**C(═O)O(CH₂)ₘNR¹¹C(═O)(CH₂)ₘ; —**C(O)O(CH₂)ₘNR¹¹C(═O)(CH₂)ₘO(CH₂)ₘ—;
   —**C(═O)O(CH₂)ₘNR¹¹C(═O)X₁ₐX₂ₐC(O)(CH₂)ₘ—;
—**C(═O)OC(R²)₂(CH₂)—NR¹¹C(O)X₁ₐX₂ₐC(═O)(CH₂)ₘ;
—**C(═O)O(CH₂)ₘNR¹¹C(═O)X₁ₐX₂ₐC(═O)(CH₂)ₘO(CH₂)—;
—**C(═O)O(CH₂)NR¹¹C(═O)X₁ₐX₂ₐC(═O)(CH₂)ₘO(CH₂)ₘC(═O)—;
—**C(═O)O(CH₂)ₘNR¹¹C(═O)XC(═O)NR¹¹(CH₂)ₘR¹¹C(═O)(CH₂)O(CH₂)ₘ;
—**C(═O)O(CH₂)ₘNR¹¹C(═O)X₅C(═O)(CH₂)ₘNR¹¹C(═O)(CH₂)ₘOC₂
—**C(═O)X₄C(═O)NR¹¹(CH₂)ₘNR¹¹C(═O)(CH₂)ₘO(CH₂)ₘ—;
—**C(═O)(CH₂)ₘNR¹¹C(═O)X₁ₐX₂ₐC(═O)(CH₂)ₘ—;
—**C(═O)O(CH₂)ₘ)C(CO)X₁ₐX₂ₐC(═O)(CH₂)₂—;
—**C(═O)(CH₂)ₘNR¹¹C(C0)((CH₂)ₘ)ₙ(CH₂)ₘ—;
   —**C(═O)O(CH₂)ₘX₆C(═O)(CH₂)ₘ—; —**C(═O)O(CH₂)ₘX₆C(═O)(CH₂)ₘ,O(CH₂)ₘ—;
—**C(═O)O(CH₂)ₘX₆C(═O)X₁ₐX₂ₐC(═O)(CH₂)ₘ—;
—**C(═O)O(CH₂)ₘX₆C(═O)X₁ₐX₂ₐC(═O)(CH₂)ₘO(CH₂)ₘ—;
—**C(O)O(CH)ₘC(═O)X₁ₐX₂C(═O)(CH₂),O(CH₂),C(═O)—;
—**C(═O)O(CH₂)ₘX₆C(═O)X₄C(═O)NR¹¹(CH₂)ₘNR¹¹C(═O)(CH₂)ₘO(CH₂)ₘ—;
—**C(═O)X₄C(═O)X₆(CH₂)ₘNR¹¹C(═O)(CH₂)ₘ,O(CH₂)ₘ—;
—**C(═O)(CH₂)ₘX₆C(═O)X₁ₐX₂ₐC(═O)(CH₂)ₘ—;
—**C(═O)O((CH₂)ₘO)ₙ(CH₂)ₘNR¹¹C(═O)X₅C(═O)(CH₂)ₘ—;
—**C(═O)O((CH₂)ₘO)(CH)ₙNR¹¹C(O)X₅C(═O)(CH₂)NR¹¹C(═O)(CH₂)ₘ—;
—**C(═O)O((CH₂)ₘO)ₙ(CH₂)ₘNR₁₁C(═O)X₅C(═O)(CH₂)ₘX₃(CH₂)ₘ—;
—**C(═O)O((CH₂)ₘO)ₙ(CH₂)ₘNR¹¹C(═O)X₅C(═O)((CH₂)ₘO)ₙ(CH₂)ₘ—;
—**C(═O)O((CH₂)ₘO)ₙ(CH₂)ₘNR¹¹C(═O)X₅C(═O)((CH₂)ₘO)ₙ(CH₂)ₘNR¹¹C(═O)(CH₂)ₘ—;
—**C(═O)O((CH₂)ₘO)ₙ(CH₂)ₘNR¹¹C(═O)X₅C(═O)((CH₂)ₘO)ₙ(CH₂)ₘNR¹¹C(═O)(CH₂)ₘX₃(CH₂)ₘ—;
—**C(═O)O((CH₂)ₘO)ₙ(CH₂)ₘNR¹¹C(═O))XC(═O)((CH₂)ₘO)ₙ(CH₂)ₘX₃(CH₂)ₘ—;
—**C(═O)O((CH₂)ₘO)ₙ(CH₂)ₘNR¹¹C(═O)X₅C(═O)(CH₂)ₘNR¹¹C(═O)((CH₂)ₘO)ₙ(CH₂)ₘ—;
—**C(═O)O((CH₂)ₘO)ₙ(CH₂)ₘNR¹¹C(═O)X₅C(═O)(CH₂)ₘNR¹¹C(═O)((CH₂)ₘO)ₙ(CH₂)ₘX₃(CH₂)ₘ—;
—**C(═O)O((CH₂)ₘO)ₙ(CH₂)ₘNR¹¹C(═O)X₅(CH₂)ₘX₃(CH₂)ₘ—;
—**C(═O)O((CH₂)ₘO)ₙ(CH₂)ₘNR¹¹C(═O)X₅((CH₂)ₘO)ₙ(CH₂)ₘ—;
—**C(═O)((CH₂)O)ₙ(CH₂)ₘNR¹¹C(═O)X((CH₂)ₘO)ₙ(CH₂)ₘNR¹¹C(═O)(CH₂)ₘ—;
—**C(═O)O((CH₂)ₘO)ₙ(CH₂)ₘNR¹¹C(═O)X((CH₂)ₘO)ₙ(CH₂)ₘNR¹¹C(═O)(CH₂)ₘX₃(CH₂)ₘ—;
—**C(═O)O((CH₂)ₘO)ₙ(CH₂)ₘNR¹¹C(═O)X((CH₂)ₘO)ₙ(CH₂)ₘX₃(CH₂)ₘ—;
—**C(═O)O((CH₂)ₘO)ₙ(CH₂)ₘNR¹¹C(═O)X₅(CH₂)ₘNR¹¹((CH₂)ₘO)ₙ(CH₂)ₘ—;
—**C(═O)O((CH₂)ₘO)ₙ(CH₂)ₘNR¹¹C(═O)X₅C(═O)(CH₂)ₘNR¹¹((CH₂)ₘO)ₙ(CH₂)ₘX₃(CH₂)ₘ—;
—**C(═O)O((CH₂)ₘO)ₙ(CH₂)ₘNR¹¹C(═O)X₅(CH₂)ₘ—;
—**C(═O)O((CH₂)ₘO)ₙ(CH₂)ₘNR¹¹C(═O)X₅C(═O)((CH₂)ₘO)ₙ(CH₂)ₘ—;
—**C(═O)O((CH₂)ₘO)ₙ(CH₂)ₘNR¹¹C(═O)X₅(CH₂)ₘX₃(CH₂)ₘ—; —**C(═O)O(CH₂)ₘ—;
—**C(═O)O((CH₂)ₘO)ₙ(CH₂)ₘ—; —**C(═O)O(CH₂)ₘNR¹¹(CH₂)ₘ—;
—**C(═O)O(CH₂)ₘNR¹¹(CH₂)ₘC(═O)X₂ₐX₁ₐC(═O)—;
—**C(═O)O(CH₂)ₘX₃(CH₂)ₘ—; —**C(═O)O((CH₂)ₘO)ₙ(CH₂)ₘX₃(CH₂)ₘ—;
—**C(═O)O((CH₂)ₘO)ₙ(CH₂)ₘNR¹¹C(═O)(CH₂)ₘ—; —**C(═O)O(CH₂)ₘNR¹¹C(═O(CH₂)ₘ,X₃(CH₂)ₘ—;
—**C(═O)O((CH₂)ₘO)ₙ(CH₂)ₘNR¹¹C(═O)(CH₂)ₘX₃(CH₂)ₘ—;
—**C(═O)O((CH₂)ₘO)ₘX₃(CH₂)ₘ—; —**C(═O)O((CH₂)ₘO)ₙ(CH₂)ₘX₃(CH₂)ₘ—;
—**C(═O)O((CH₂)ₘO)ₙ(CH₂)ₘC(═O)NR¹¹(CH₂)ₘ—; —**C(═O)O(CH₂)ₘC(R¹²)₂—;
—**C(═O)OCH₂)ₘC(R¹²)₂SS(CH₂)ₘNR¹¹C(═O)(CH₂)ₘ—, and
—**C(═O)O(CH₂)ₘC(═O)NR¹¹(CH₂)ₘ—, where: ** indicates point of attachment to the drug moiety (D) and the other end can be connected to R¹⁰⁰, i.e., the coupling group as described herein;
   wherein:
   X₁ₐ is

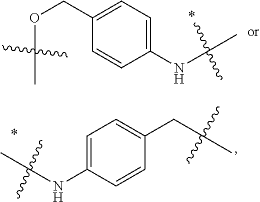 [SMILES: *Nc1ccc(CO*)cc1 ; *Cc1ccc(N*)cc1]

where the * indicates the point of attachment to X₂ₐ;

X₂ₐ is selected from

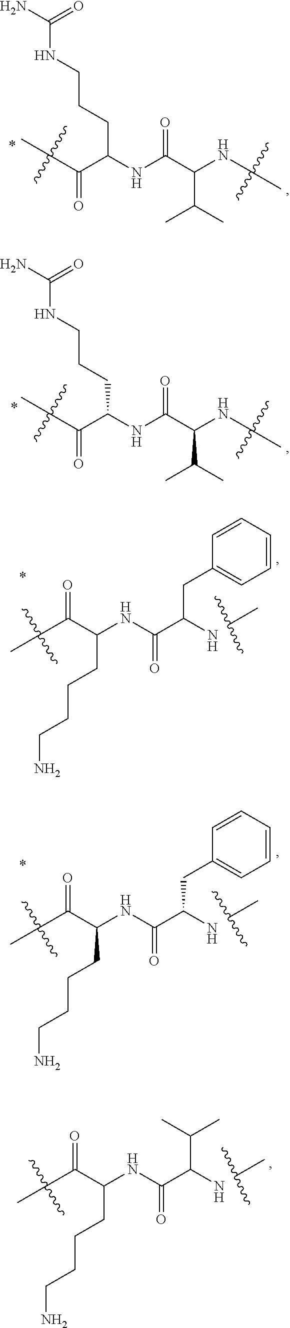

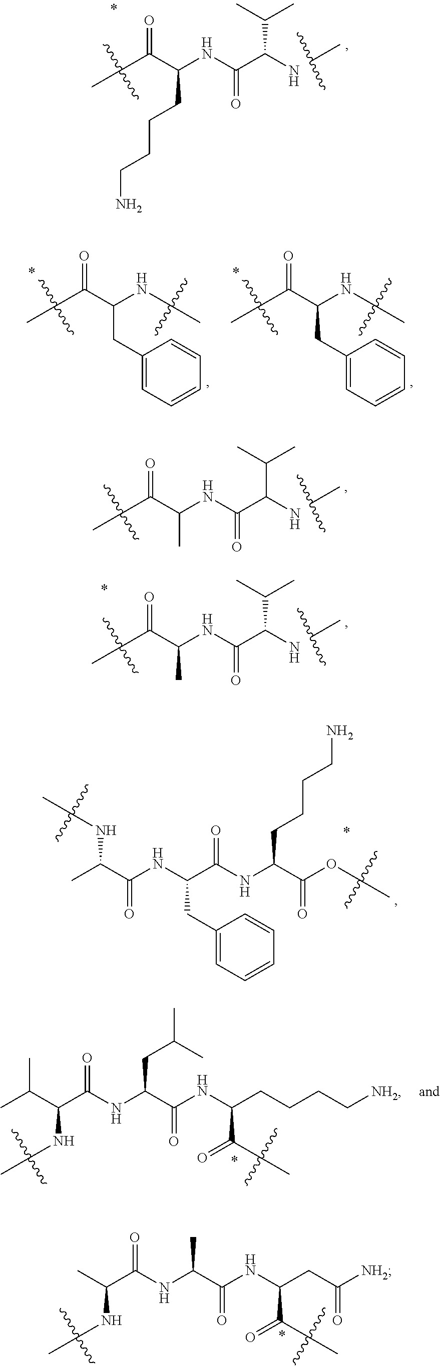

where the * indicates the point of attachment to X₁ₐ;
X₃ is

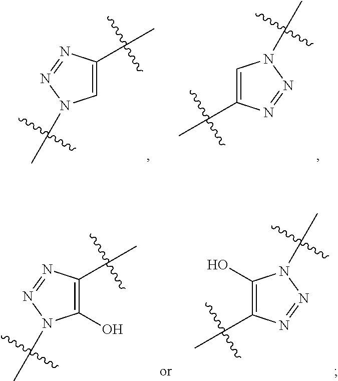 [SMILES: *c1cn(*)nn1 ; *c1cn(*)nn1 ; *c1nnn(*)c1O ; *c1nnn(*)c1O]

X₄ is —O(CH₂)ₙSSC(R¹²)₂(CH₂)ₙ— or —(CH₂)ₙC(R¹²)₂SS(CH₂)ₙO—;
X₅ is

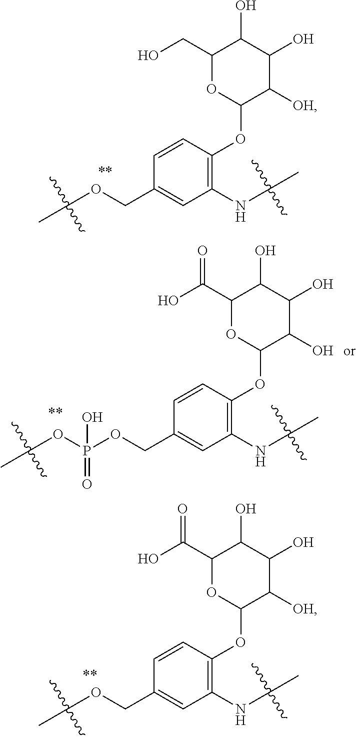 [SMILES: *Nc1cc(CO*)ccc1OC1OC(CO)C(O)C(O)C1O ; *Nc1cc(COP(=O)(O)O*)ccc1OC1OC(C(=O)O)C(O)C(O)C1O ; *Nc1cc(CO*)ccc1OC1OC(C(=O)O)C(O)C(O)C1O]

where the ** indicates orientation toward the Drug moiety;
X₆ is

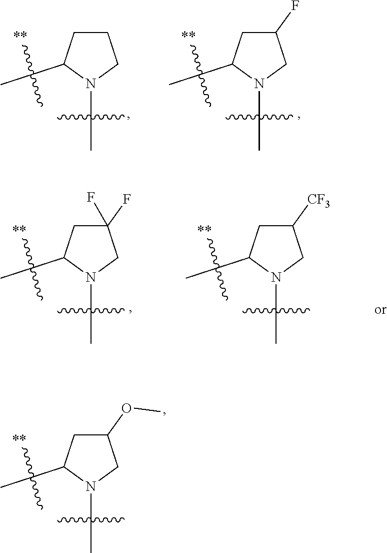

where the ** indicates orientation toward the Drug moiety;
each R¹¹ is independently selected from H and C₁-C₆alkyl;
each R¹² is independently selected from H and C₁-C₆alkyl;
each m is independently selected from 1, 2, 3, 4, 5, 6, 7, 8, 9 and 10, and
each n is independently selected from 1, 2, 3, 4, 5, 6, 7, 8, 9, 10, 11, 12, 13, 14, 15, 16,17 and 18.

### Methods of Conjugation

The present invention provides various methods of conjugating Linker-Drug groups of the invention to antibodies or antibody fragments to produce Antibody Drug Conjugates which comprise a conjugate linker having one or more hydrophilic moieties.

A general reaction scheme for the formation of Antibody Drug Conjugates of Formula (E′) is shown in Scheme 2 below:

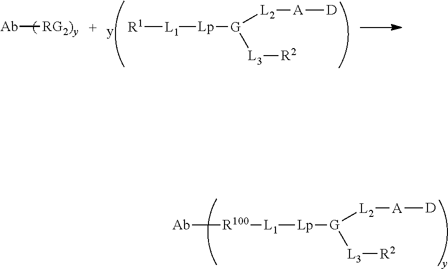

where: RG₂ is a reactive group which reacts with a compatible R¹ group to form a corresponding R¹⁰⁰ group (such groups are illustrated in Table 8 and Table 9). D, R¹, L₁, Lp, L₂, L₃, R², A, G, Ab, y and R¹⁰⁰are as defined herein.

Scheme 3 further illustrates this general approach for the formation of Antibody Drug Conjugates of Formula (E′), wherein the antibody comprises reactive groups (RG₂) which react with an R¹ group (as defined herein) to covalently attach the Linker-Drug group to the antibody via an R¹⁰⁰ group (as defined herein). For illustrative purposes only Scheme 3 shows the antibody having four RG₂ groups.

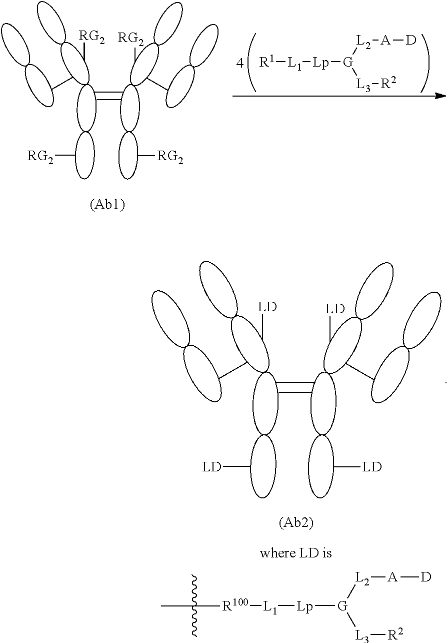

In one aspect, Linker-Drug groups are conjugated to antibodies via modified cysteine residues in the antibodies (see for example WO2014/124316). Scheme 4 illustrates this approach for the formation of Antibody Drug Conjugates of Formula (E′) wherein a free thiol group generated from the engineered cysteine residues in the antibody react with an R¹ group (where R¹ is a maleimide) to covalently attach the Linker-Drug group to the antibody via an R¹⁰⁰ group (where R¹⁰⁰ is a succinimide ring). For illustrative purposes only Scheme 4 shows the antibody having four free thiol groups.

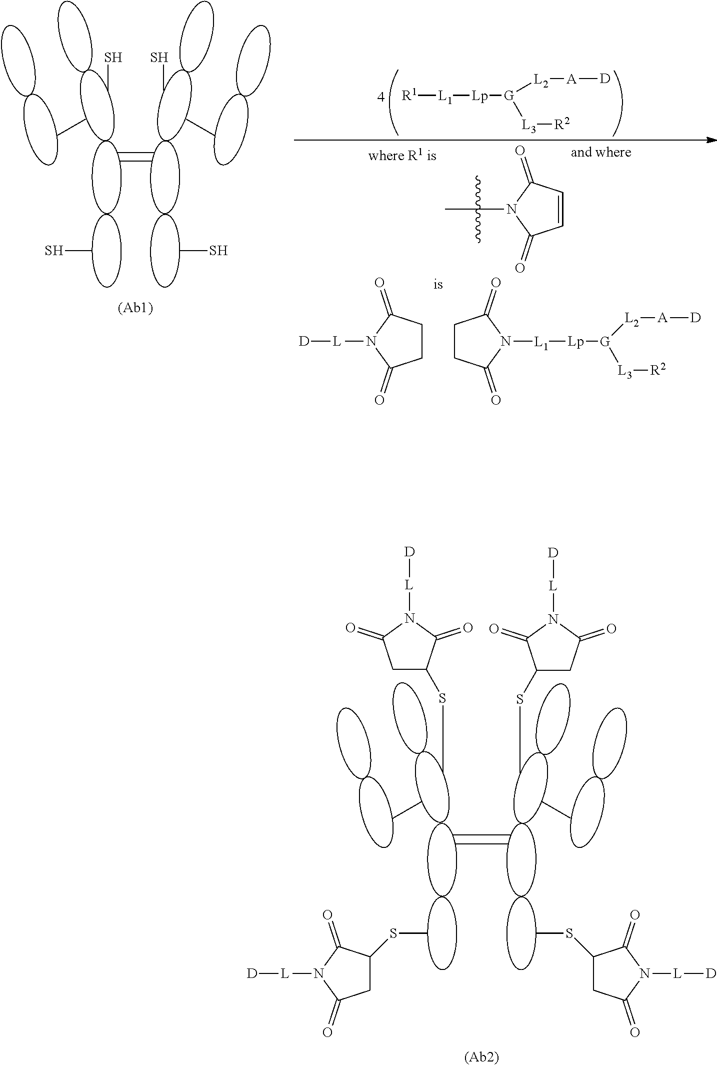

In another aspect, Linker-Drug groups are conjugated to antibodies via lysine residues in the antibodies. Scheme 5 illustrates this approach for the formation of Antibody Drug Conjugates of Formula (E′) wherein a free amine group from the lysine residues in the antibody react with an R¹ group (where R¹ is an NHS ester, a pentafluorophenyl or a tetrafluorophenyl) to covalently attach the Linker-Drug group to the antibody via an R¹⁰⁰ group (where R¹⁰⁰ is an amide). For illustrative purposes only Scheme 5 shows the antibody having four amine groups.

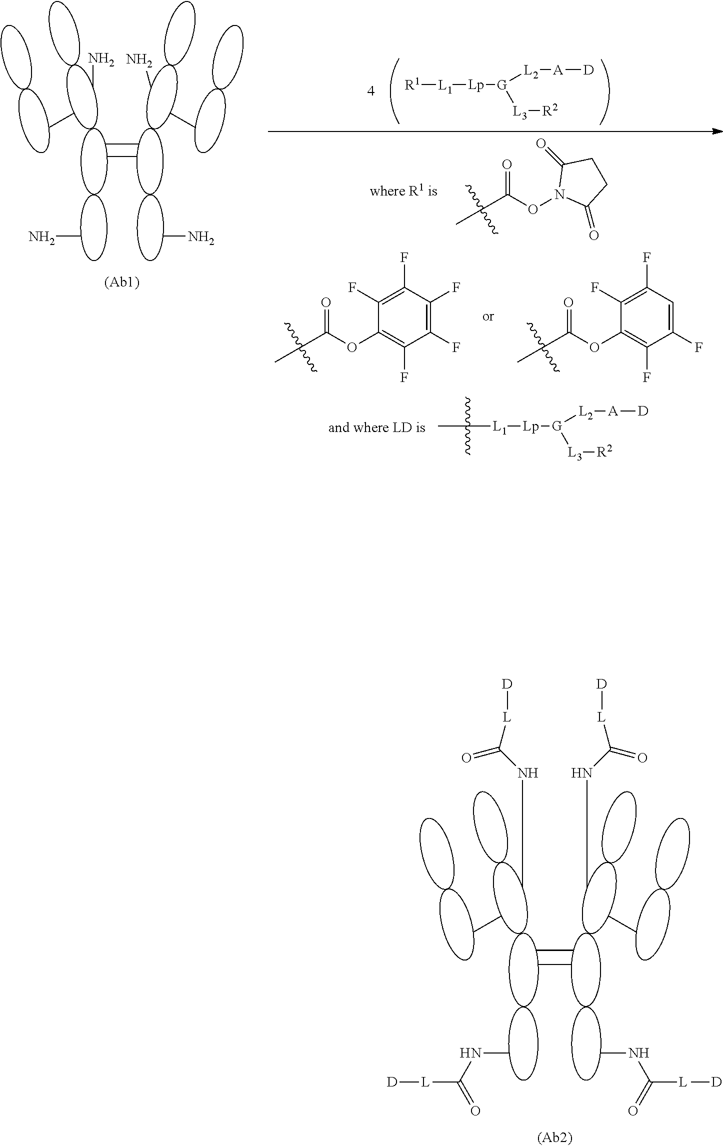

In another aspect, Linker-Drug groups are conjugated to antibodies via formation of an oxime bridge at the naturally occurring disulfide bridges of an antibody. The oxime bridge is formed by initially creating a ketone bridge by reduction of an interchain disulfide bridge of the antibody and re-bridging using a 1,3-dihaloacetone (e.g. 1,3-dichloroacetone). Subsequent reaction with a Linker-Drug group comprising a hydroxyl amine thereby form an oxime linkage (oxime bridge) which attaches the Linker-Drug group to the antibody (see for example WO2014/083505). Scheme 6 illustrates this approach for the formation of Antibody Drug Conjugates of Formula (E′).

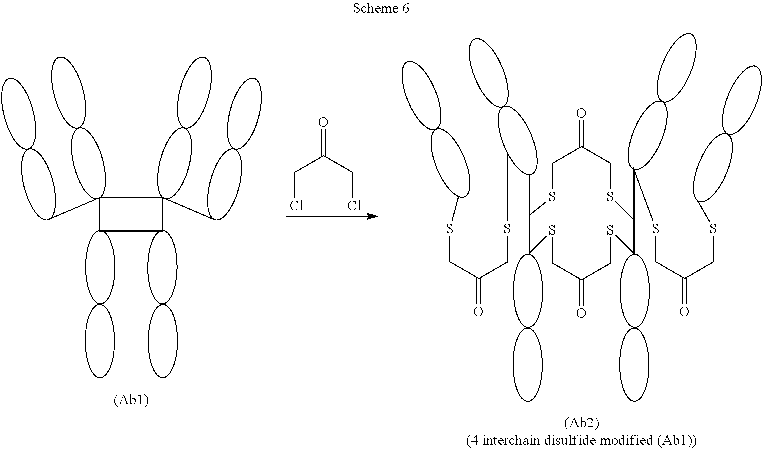

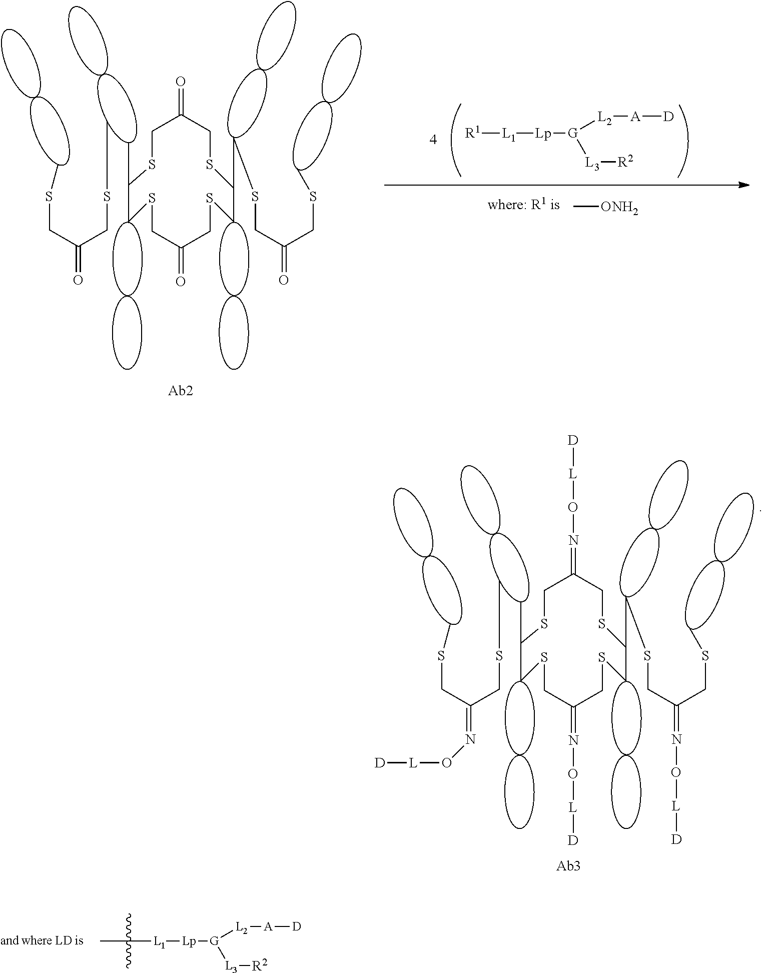

A general reaction scheme for the formation of Antibody Drug Conjugates of Formula (F′) is shown in Scheme 7 below:

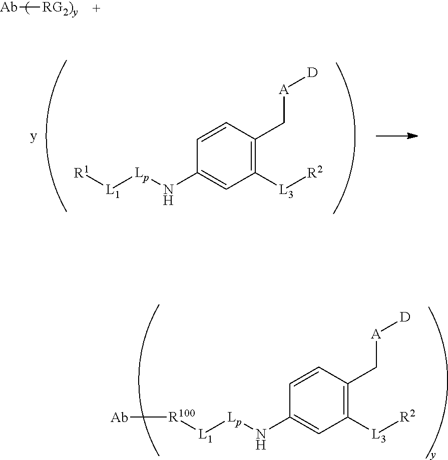

where: RG₂ is a reactive group which reacts with a compatible R¹ group to form a corresponding R¹⁰⁰ group (such groups are illustrated in Table 8 and Table 9). D, R¹, L₁, Lp, Ab, y and R¹⁰⁰are as defined herein.

Scheme 8 further illustrates this general approach for the formation of Antibody Drug Conjugates of Formula (F′), wherein the antibody comprises reactive groups (RG₂) which react with an R¹ group (as defined herein) to covalently attach the Linker-Drug group to the antibody via an R¹⁰⁰ group (as defined herein). For illustrative purposes only Scheme 8 shows the antibody having four RG₂ groups.

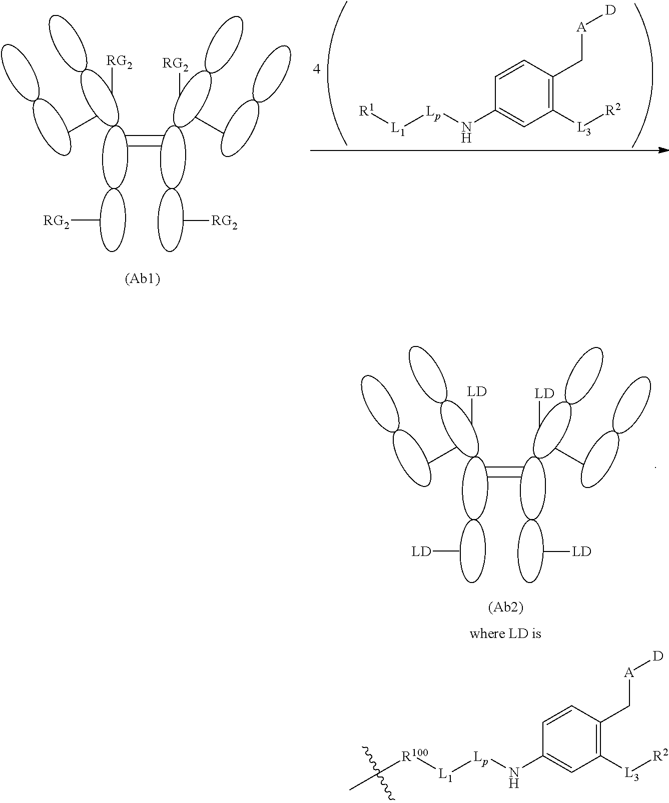

In one aspect, Linker-Drug groups are conjugate to antibodies via modified cysteine residues in the antibodies (see for example WO2014/124316). Scheme 9 illustrates this approach for the formation of Antibody Drug Conjugates of Formula (F′) wherein a free thiol group generated from the engineered cysteine residues in the antibody react with an R¹ group (where R¹ is a maleimide) to covalently attach the Linker-Drug group to the antibody via an R¹⁰⁰ group (where R¹⁰⁰ is a succinimide ring). For illustrative purposes only Scheme 9 shows the antibody having four free thiol groups.

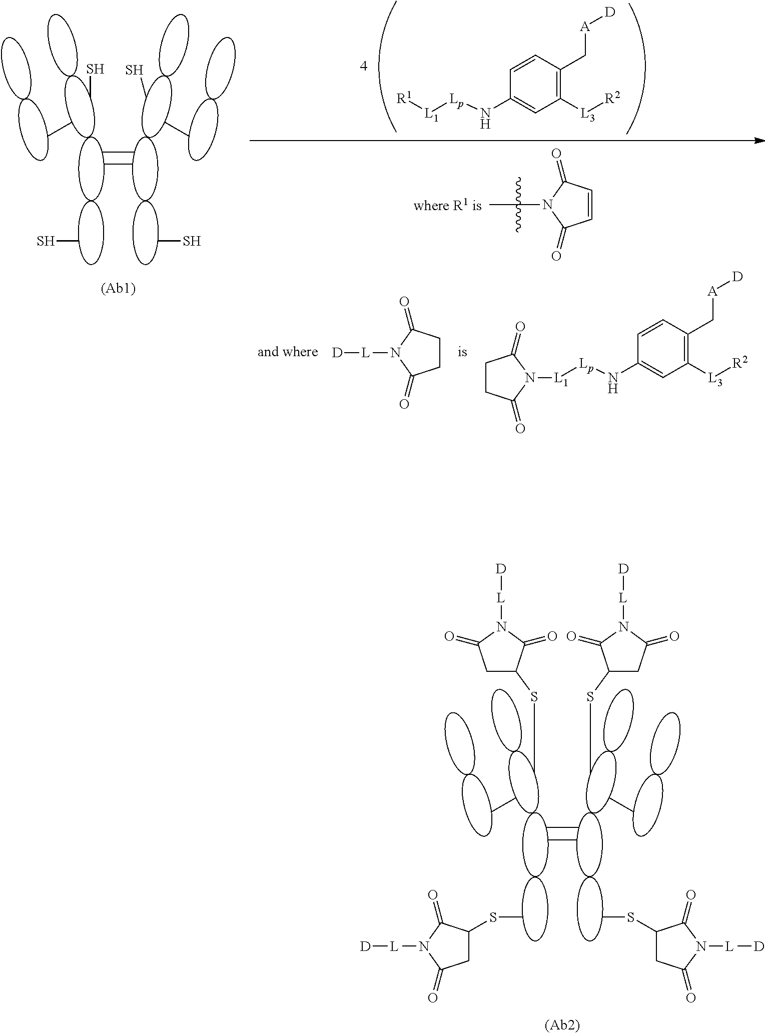

In another aspect, Linker-Drug groups are conjugated to antibodies via lysine residues in the antibodies. Scheme 10 illustrates this approach for the formation of Antibody Drug Conjugates of Formula (F′) wherein a free amine group from the lysine residues in the antibody react with an R¹ group (where R¹ is an NHS ester, a pentafluorophenyl or a tetrafluorophenyl) to covalently attach the Linker-Drug group to the antibody via an R¹⁰⁰ group (where R¹⁰⁰ is an amide). For illustrative purposes only Scheme 10 shows the antibody having four amine groups.

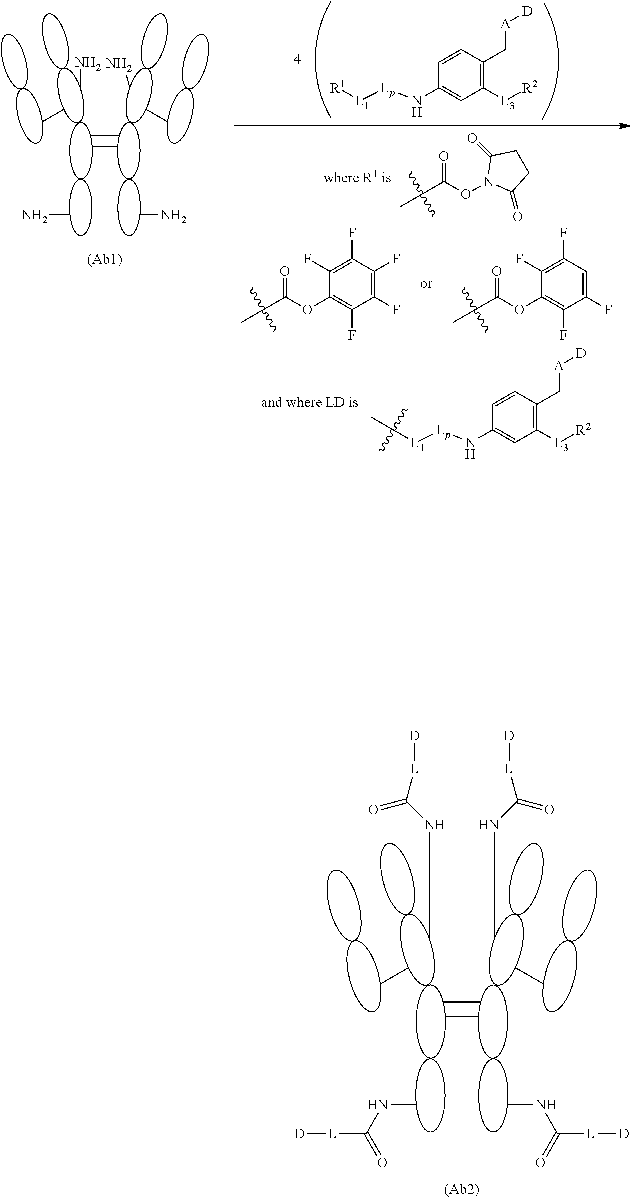

In another aspect, Linker-Drug groups are conjugated to antibodies via formation of an oxime bridge at the naturally occurring disulfide bridges of an antibody. The oxime bridge is formed by initially creating a ketone bridge by reduction of an interchain disulfide bridge of the antibody and re-bridging using a 1,3-dihaloacetone (e.g. 1,3-dichloroacetone). Subsequent reaction with a Linker-Drug group comprising a hydroxyl amine thereby form an oxime linkage (oxime bridge) which attaches the Linker-Drug group to the antibody (see for example WO2014/083505). Scheme 11 illustrates this approach for the formation of Antibody Drug Conjugates of Formula (F′).

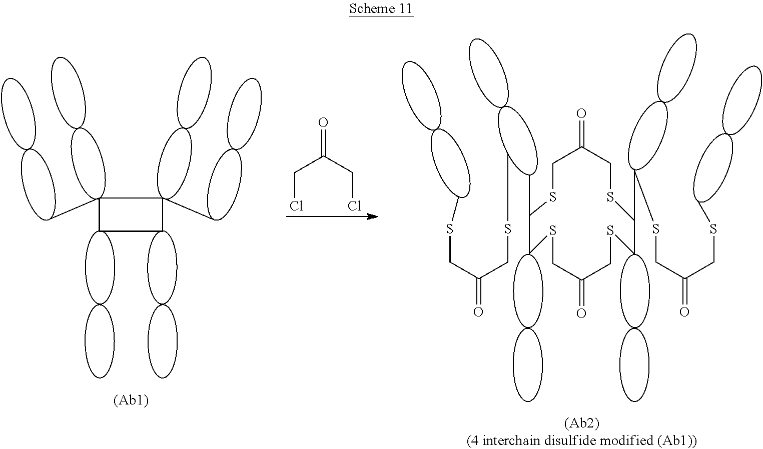

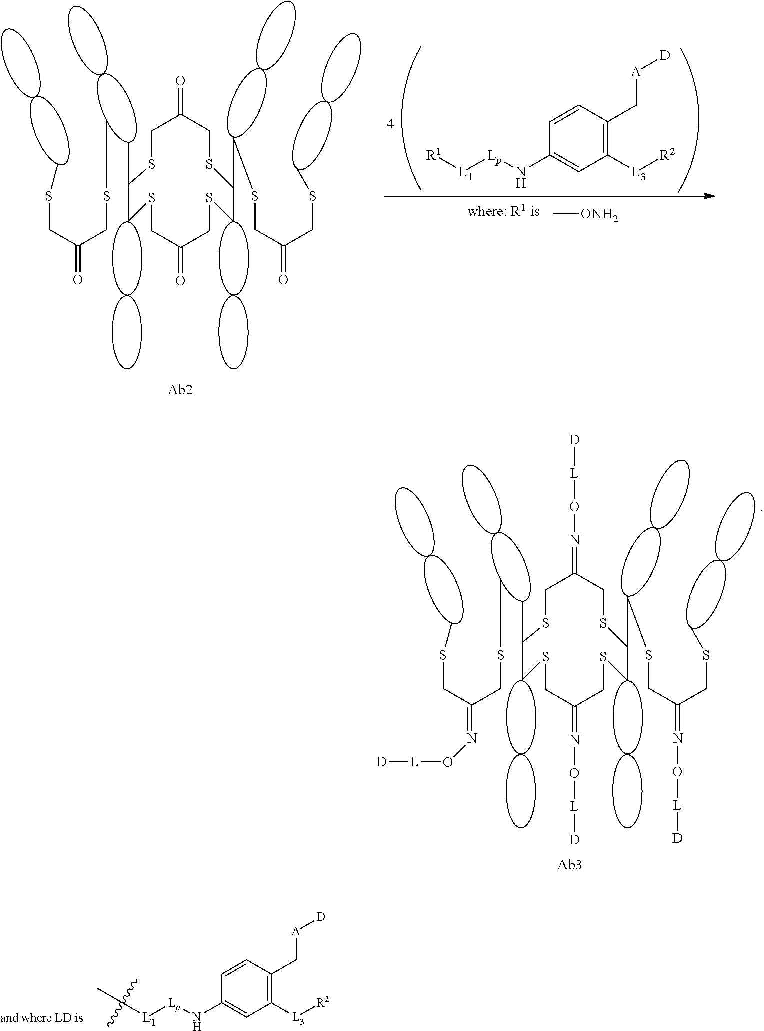

Provided are also protocols for some aspects of analytical methodology for evaluating antibody conjugates of the invention. Such analytical methodology and results can demonstrate that the conjugates have favorable properties, for example properties that would make them easier to manufacture, easier to administer to patients, more efficacious, and/or potentially safer for patients. One example is the determination of molecular size by size exclusion chromatography (SEC) wherein the amount of desired antibody species in a sample is determined relative to the amount of high molecular weight contaminants (e.g., dimer, multimer, or aggregated antibody) or low molecular weight contaminants (e.g., antibody fragments, degradation products, or individual antibody chains) present in the sample. In general, it is desirable to have higher amounts of monomer and lower amounts of, for example, aggregated antibody due to the impact of, for example, aggregates on other properties of the antibody sample such as but not limited to clearance rate, immunogenicity, and toxicity. A further example is the determination of the hydrophobicity by hydrophobic interaction chromatography (HIC) wherein the hydrophobicity of a sample is assessed relative to a set of standard antibodies of known properties. In general, it is desirable to have low hydrophobicity due to the impact of hydrophobicity on other properties of the antibody sample such as but not limited to aggregation, aggregation over time, adherence to surfaces, hepatotoxicity, clearance rates, and pharmacokinetic exposure. See Damle, N. K., Nat Biotechnol. 2008; 26(8):884-885; Singh, S. K., Pharm Res. 2015; 32(11):3541-71. When measured by hydrophobic interaction chromatography, higher hydrophobicity index scores (i.e. elution from HIC column faster) reflect lower hydrophobicity of the conjugates. As shown in Examples below, a majority of the tested antibody conjugates showed a hydrophobicity index of greater than 0.8. In some embodiments, provided are antibody conjugates having a hydrophobicity index of 0.8 or greater, as determined by hydrophobic interaction chromatography.

### EXAMPLES

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The examples provided do not in any way limit the disclosure.

### Example 1. Synthesis and Characterization of PanRAS Payloads

Exemplary payloads were synthesized using exemplary methods described in WO2021/091956 or WO2022/060836, where are hereby incorporated by reference in their entirety. All reagents obtained from commercial sources were used without further purification. Anhydrous solvents were obtained from commercial sources and used without further drying.

### Example 2. Synthesis and Characterization of Conjugate Linkers

Exemplary conjugate linkers and precursors thereof were synthesized using exemplary methods described in PCT Application No. PCT/US2021/060620, where is hereby incorporated by reference in their entirety.

### Example 3. Synthesis and Characterization of Linker-Payloads, and Precursors Thereof

Exemplary linkers, linker-payloads, and precursors thereof were synthesized using exemplary methods described in this example.

### ABBREVIATIONS

DCC: dicyclohexylcarbodiimide
DCE: dichloroethane
DCM: dichloromethane
DIEA/DIPEA: N,N-Diisopropylethylamine
DMTMM: 4-(4,6-dimethoxy-1,3,5-triazin-2-yl)-4-methyl-morpholinium chloride
DMF: dimethylformamide
DMSO: dimethylsulfoxyde
EDC/EDC-HCl: 1-Ethyl-3-(3-dimethylaminopropyl)carbodiimide hydrochloride
HATU: 1-[Bis(dimethylamino)methylene]-1H-1,2,3-triazolo[4,5-b]pyridinium 3-oxide hexafluorophosphate
HBTU: (2-(1H-benzotriazol-1-yl)-1,1,3,3-tetramethyluronium hexafluorophosphate
HOAt: 1-Hydroxy-7-azabenzotriazole
MeCN: acetonitrile
MeOH: methanol
NMP: N-methylpyrrolidine
RT: room temperature
TES: triethylsilane
TBAF: tetrabutyl ammonium fluoride
TBAI: tetrabutyl ammonium iodide
TBTU: [Bis(dimethylamino)methylene]-1H-benzotriazolium 3-Oxide Tetrafluoroborate
pTsOH: para-toluene sulfonic acid
PyBOP: benzotriazol-1-yloxytripyrrolidinophosphonium hexafluorophosphate
TSTU: O—(N-Succinimidyl)-N,N,N′,N′-tetramethyluronium tetrafluoroborate
TH F: tetrahydrofuran
TFA: trifluoroacetic acid
TFE: 2,2,2-Trifluoroethanol

### Materials, Methods & General Procedures

All reagents obtained from commercial sources were used without further purification. Anhydrous solvents were obtained from commercial sources and used without further drying. Flash chromatography was performed on CombiFlash Rf (Teledyne ISCO) with pre-packed silica-gel cartridges (Macherey-Nagel Chromabond Flash). Thin layer chromatography was conducted with 5×10 cm plates coated with Merck Type 60 F254 silica-gel. Microwave heating was performed in CEM Discover® instrument.

NMR data were acquired at a temperature of 298K on a Bruker Avance NMR spectrometer equipped with a 5 mm BBFO CryoProbe with z-gradient operating at a frequency of 400.13 MHz for ¹H, 376.50 MHz for ¹⁹F, 100.61 MHz for ¹³C. Chemical shifts for the ¹H and ¹³C spectra were referenced by setting internal tetramethylsilane (TMS) to 0 ppm.

### Analytical Methods

LC/MS data was acquired using an instrument with the following parameters:

| | |
|---|---|
| Pump | Waters AcQuity UPLC Binary Solvent |
| | Manager |
| Sample Manager | Waters AcQuity UPLC Sample Manager |
| Column | Waters AcQuity UPLC Column Manager |
| Compartment | |
| Detector | Waters AcQuity UPLC PDA |
| ELSD | Shimadzu ELSD-LTII |
| Mass Spec | Waters SQD |
| Columns | AcQuity UPLC BEH C18 1.7 μm 2.1 × 50 mm |
| Eluent A1 | 0.1% Formic Acid in Water |
| Eluent B1 | 0.1% Formic Acid in Acetonitrile |
| Eluent A2 | 5 mM Ammonium Hydroxide in Water |
| Eluent B2 | 5 mM Ammonium Hydroxide in Acetonitrile |

The methods used to generate LC/MS data were as follows:

| Eluent A1 | 0.1% Formic Acid in Water | | |
|---|---|---|---|
| Eluent B1 | 0.1% Formic Acid in Acetonitrile | | |
| Flow | 1.0 mL/min | | |
| Stop Time | 3.00 min | | |
| pH | 2.6 | | |
| | | % A | % B |
| | | (Eluent | (Eluent |
| Gradient | Time | A1) | B1) |
| | 0 | 95 | 5 |
| | 0.2 | 95 | 5 |
| | 2 | 5 | 95 |
| | 2.5 | 5 | 95 |
| | 2.6 | 95 | 5 |
| | 3 | 95 | 5 |

| | |
|---|---|
| Column | AcQuity UPLC BEH C18 |
| | 1.7 μm 2.1 × 50 mm |
| Column | 50° C. |
| Temperature | |
| TAC | 210-400 nm |
| Mass Range | 120-1500 Da |
| Scan Time | 0.3 sec |

### 2 Min Basic Method:

| Eluent A1 | 5 mM Ammonium | | |
|---|---|---|---|
| | Hydroxide in Water | | |
| Eluent B1 | 5 mM Ammonium | | |
| | Hydroxide in Acetonitrile | | |
| Flow | 1.0 mL/min | | |
| Stop Time | 3.00 min | | |
| pH | 10.2 | | |
| | | % A | % B |
| | | (Eluent | (Eluent |
| Gradient | Time | A1) | B1) |
| | 0 | 95 | 5 |
| | 0.2 | 95 | 5 |
| | 2 | 5 | 95 |
| | 2.5 | 5 | 95 |
| | 2.6 | 95 | 5 |
| | 3 | 95 | 5 |

| | |
|---|---|
| Column | AcQuity UPLC BEH C18 |
| | 1.7 μm 2.1 × 50 mm |
| Column | 50° C. |
| Temperature | |
| TAC | 210-400 nm |
| Mass Range | 120-1500 Da |
| Scan Time | 0.3 sec |

### 5 Min Acidic Method

| Flow | 1.0 mL/min | | |
|---|---|---|---|
| Stop Time | 5.20 min | | |
| pH | 2.6 | | |
| | | % A | % B |
| | | (Eluent | (Eluent |
| Gradient | Time | A1) | B1) |
| | 0 | 98 | 2 |
| | 4.4 | 2 | 98 |
| | 5.15 | 2 | 98 |
| | 5.19 | 98 | 2 |

| | |
|---|---|
| Column | AcQuity UPLC BEH C18 |
| | 1.7 μm 2.1 × 50 mm |
| Column | 50° C. |
| Temperature | |
| TAC | 210-400 nm |
| Mass Range | 120-1500 Da |
| Scan Time | 0.3 sec |

HRMS data was acquired using an instrument with the following parameters:

### HRMS_QT01

| | |
|---|---|
| Pump | Waters AcQuity UPLC Binary |
| | Solvent Manager |
| Sample Manager | Waters AcQuity UPLC Sample Manager |
| Column | Waters AcQuity UPLC Column Manager |
| Compartment | |
| Detector | Waters AcQuity UPLC PDA |
| ELSD | n/a |
| Mass Spec | Waters Xevo G2 Qtof |
| Columns | AcQuity UPLC PrST C4 300Å 1.7 μm |
| | 2.1 × 100 mm |
| | AcQuity UPLC CSH C18 1.7 μm 2.1 × 50 mm |
| | ProSwift RP-3U 4.6 × 50 mm SS |
| Eluent A1 | 0.1% Formic Acid in Water |
| Eluent B1 | 0.1% Formic Acid in Acetonitrile |
| Eluent A2 | 0.05% Triflouroacetic Acid in Water |
| Eluent B2 | 0.05% Triflouroacetic Acid in Acetonitrile |

### HRMS_QTO2

| | |
|---|---|
| Pump | Waters AcQuity UPLC Binary Solvent |
| Sample Manager | Manager |
| | Waters AcQuity UPLC Sample Manager |
| Column | Waters AcQuity UPLC Column Manager |
| Compartment | |
| Detector | Waters AcQuity UPLC PDA |
| ELSD | n/a |
| Mass Spec | Waters Xevo G2 Qtof |
| Columns | AcQuity UPLC PrST C4 300Å 1.7 μm |
| | 2.1 × 100 mm |
| | AcQuity UPLC CSH C18 1.7 μm 2.1 × 50 mm |
| | POROS R1 10 um 2.1 × 100 mm |
| Eluent A1 | 0.1% Formic Acid in Water |
| Eluent B1 | 0.1% Formic Acid in 80:20 |
| | Isopropanol:Acetonitrile |
| Eluent A2 | 0.05% Triflouroacetic Acid in Water |
| Eluent B2 | 0.05% Triflouroacetic Acid in Acetonitrile |

The method used to generate HRMS data for linker/payloads and synthetic intermediates was as follows:

Peptide_300-4000_Da_5min QT1

| Flow | 1.0 mL/min | | |
|---|---|---|---|
| Stop Time | 5.2 min | | |
| pH | 2.6 | | |
| | | % A | % B |
| Gradient | Time | (Eluent | (Eluent B2) |
| | 0 | 98 | 2 |
| | 4.4 | 2 | 98 |
| | 5.15 | 2 | 98 |
| | 5.19 | 98 | 2 |

| | |
|---|---|
| Column | AcQuity UPLC BEH C18 |
| | 1.7 μm 2.1 × 50 mm |
| Column Temp | 500° C. |
| TAC | 210-400 nm |
| Mass Range | 300-4000 Da |
| Processing | n/a |
| Scan Time | 0.5 sec |

Peptide_300-10000_Da_5min QT1

| Flow | 1.0 mL/min | | |
|---|---|---|---|
| Stop Time | 5.2 min | | |
| pH | 2.6 | | |
| | | % A | %B |
| | | (Eluent | (Eluent |
| Gradient | Time | A2) | B2) |
| | 0 | 98 | 2 |
| | 4.4 | 2 | 98 |
| | 5.15 | 2 | 98 |
| | 5.19 | 98 | 2 |

| | |
|---|---|
| Column | AcQuity UPLC BEH C18 |
| | 1.7 μm 2.1 × 50 mm |
| Column Temp | 50° C. |
| TAC | 210-400 nm |
| Mass Range | 300-4000 Da |
| Processing | 300-10000 Da |
| Range | 0.5 sec |
| Scan Time | |

Peptide_300-4000_Da_5min QT2

| Flow | 1.0 mL/min | | |
|---|---|---|---|
| Stop Time | 5.2 min | | |
| pH | 2.6 | | |
| | | % A | % B |
| | | (Eluent | (Eluent |
| Gradient | Time | A2) | B2) |
| | 0 | 98 | 2 |
| | 4.4 | 2 | 98 |
| | 5.15 | 2 | 98 |
| | 5.19 | 98 | 2 |

| | |
|---|---|
| Column | AcQuity UPLC BEH C18 |
| | 1.7 μm 2.1 × 50 mm |
| Column Temp | 80° C. |
| TAC | 210-400 nm |
| Mass Range | 300-4000 Da |
| Processing | n/a |
| Range | |
| Scan Time | 0.2 sec |

Peptide_300-10000_Da_5min QT2

| Flow | 1.0 mL/min | | |
|---|---|---|---|
| Stop Time | 5.2 min | | |
| pH | 2.6 | | |
| | | % A | % B |
| Gradient | Time | (Eluent | (Eluent B2) |
| | 0 | 98 | 2 |
| | 4.4 | 2 | 98 |
| | 5.15 | 2 | 98 |
| | 5.19 | 98 | 2 |

| | |
|---|---|
| Column | AcQuity UPLC BEH C18 |
| | 1.7 μm 2.1 × 50 mm |
| Column Temp | 80° C. |
| TAC | 210-400 nm |
| Mass Range | 300-4000 Da |
| Processing | 300-10000 Da |
| Scan Time | 0.2 sec |

### Preparative RP-HPLC:

Preparative-HPLC (“Prep-HPLC”) data were acquired using Teledyne ISCO purification systems using 018 or 04 RP ISCO or ISCO-gold columns.

Four Prep-HPLC methods were used:
a. TFA method: solvent: A water+0.05% TFA, B acetonitrile+0.05% TFA, gradient from 5 to 100% B in 15 to 30 CV
b. NH₄HCO₃ method: solvent: A water+0.02 M NH₄HCOs, B acetonitrile/water 80/20+0.02 M NH₄HCOs, gradient from 5 to 100% B in 15 to 30 CV
c. Neutral method: solvent: A water, B acetonitrile, gradient from 5 to 100% B in 15 to 30 CV
d. Formic Acid method: solvent: A water+0.05% Formic Acid, B acetonitrile+0.05% Formic Acid, gradient from 5 to 100% B in 15 to 30 CV

Gradient variations of methods a.-d. were employed as appropriate. All the fractions containing the pure compound were combined and directly freeze-dried to afford the compound as an amorphous powder.

### Synthesis of 1-(4-((S)-2-((S)-2-((tert-butoxycarbonyl)amino)-3-methylbutanamido)-5-ureidopentanamido)benzyl)-4-(5-((6³S,4S)-1¹-ethyl-10,10-dimethyl-4-((S)-3-methyl-2-((2R,3R)—N-methyl-3-phenyltetrahydrofuran-2-carboxamido)butanamido)-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-12-yl)-6-((S)-1-methoxyethyl)pyridin-3-yl)-1-methylpiperazin-1-ium trifluoroacetate

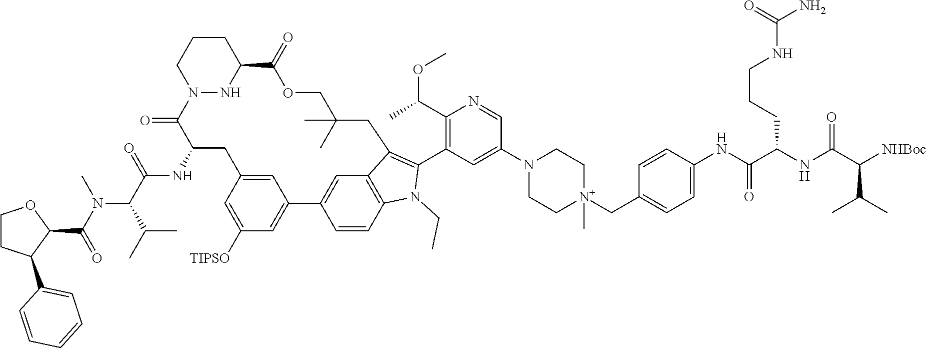

To a mixture of (2R,3R)—N-((2S)-1-(((6³S,4S)-1¹-ethyl-12~(2~((S)-1-methoxyethyl)-5~(4-nethylpiperazin-1-yl)pyridin-3-yl)-10,10-dimethyl-5,7-dioxo-2-((triisopropylsilyl)oxy)-6¹,6²,6³,6⁴,6⁵,6⁶-hexahydro-1′H-8-oxa-1(5,3)-indola-6(1,3)-pyridazina-2(1,3)-benzenacycloundecaphane-4-yl)amino)-3-methyl-1-oxobutan-2-yl)-N-methyl-3-phenyltetrahydrofuran-2-carboxamide formate (200 mg, 154.4 μmol), tert-butyl ((S)-1~(((S)-1-((4-(chlororethyl)phenyl)amino)-1-oxo-5-ureidopentan-2-yl)amino)-3-methyl-1-oxobutan-2-yl)carbamate (100 mg, 201 μmol) and sodium iodide (23.1 mg, 154 μmol) in DMSO (2.5 mL) was added DIEA (81 μL, 463 μmol). After stirring for 6 hours the solution was diluted with DMSO (3 mL) and was directly purified by ISCO gold RP-HPLC. After lyophilization, 1-(4-((S)-2-((S)-2-((tert-butoxycarbonyl)amino)-3-methylbutanamido)-5-ureidopentanamido)benzyl)-4-(5-((6³S,4S)-1¹-ethyl-10,10-dimethyl-4-((S)-3-methyl-2-((2R,3R)—N-methyl-3-phenyltetrahydrofuran-2-carboxamido)butanamido)-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)-1-methylpiperazin-1-ium trifluoroacetate (217 mg, 79% yield) was obtained. HRMS: M+=1642.9900, Rt=3.11 min. 5 min acidic method.

### Synthesis of 1-(4-((S)-2-((S)-2-((tert-butoxycarbonyl)amino)-3-methylbutanamido)-5-ureidopentanamido)benzyl)-4-(5-((6³S,4S)-1¹-ethyl-2⁵-hydroxy-10,10-dimethyl-4-((S)-3-methyl-2-((2R,3R)—N-methyl-3-phenyltetrahydrofuran-2-carboxamido)butanamido)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)-1-methylpiperazin-1-ium trifluoroacetate

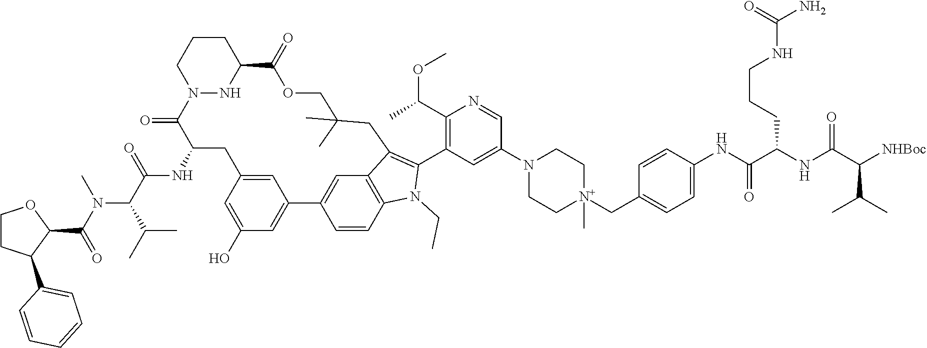 [SMILES: CCn1c(-c2cc(N3CC[N+](C)(Cc4ccc(NC(=O)[C@H](CCCNC(N)=O)NC(=O)[C@@H](NC(=O)OC(C)(C)C)C(C)C)cc4)CC3)cnc2[C@H](C)OC)c2c3cc(ccc31)-c1cc(O)cc(c1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2]

To a solution of 1-(4-((S)-2-((S)-2-((tert-butoxycarbonyl)amino)-3-methylbutanamido)-5-ureidopentanamido)benzyl)-4-(5-((6³S,4S)-1¹-ethyl-10,10-dimethyl-4-((S)-3-methyl-2-((2R,3R)—N-methyl-3-phenyltetrahydrofuran-2-carboxamido)butanamido)-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)-1-methylpiperazin-1-ium trifluoroacetate (217 mg, 123.4 μmol) in NMP (1.25 mL) was added 1.0 M TBAF in THF (248 μL, 248 μmol). After stirring for 30 minutes, the solution was diluted with DMSO (4 mL) and was directly purified by ISCO gold RP-HPLC. After lyophilization, 1-(4-((S)-2-((S)-2-((tert-butoxycarbonyl)amino)-3-methylbutanamido)-5-ureidopentanamido)benzyl)-4-(5-((6³S,4S)-1¹-ethyl-2⁵-hydroxy-10,10-dimethyl-4-((S)-3-methyl-2-((2R,3R)—N-methyl-3-phenyltetrahydrofuran-2-carboxamido)butanamido)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)-1-methylpiperazin-1-ium trifluoroacetate (198 mg, 90% yield) was obtained HRMS: M+=1486.8500, Rt=225 min, 5 min acidic method. Material contained some Bu4N+ salt impurity that was carried on to next step.

### Synthesis of 1-(4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-4-(5-((6³S,4S)-1¹-ethyl-2⁵-hydroxy-10,10-dimethyl-4-((S)-3-methyl-2-((2R,3R)—N-methyl-3-phenyltetrahydrofuran-2-carboxamido)butanamido)-5,7-dioxo-6¹,6²,6³,6⁴,6⁵,6⁶-hexahydro-1′H-8-oxa-1(5,3)-indola-6(1,3)-pyridazina-2(1,3)-benzenacycloundecaphane-1²-yl)-6-((S)-1-methoxyethyl)pyridin-3-yl)-1-methylpiperazin-1-ium trifluoroacetate (L102-D101)

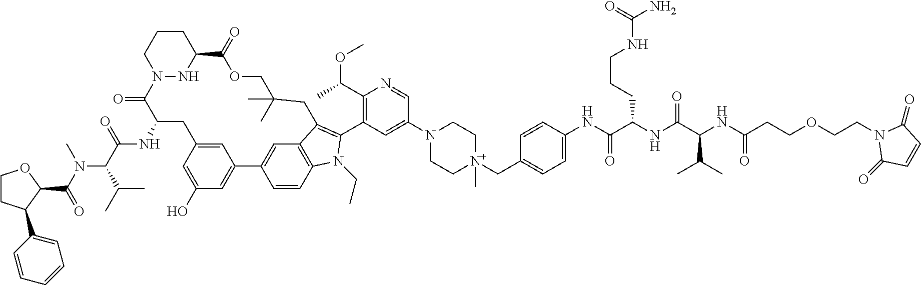 [SMILES: CCn1c(-c2cc(N3CC[N+](C)(Cc4ccc(NC(=O)[C@H](CCCNC(N)=O)NC(=O)[C@@H](NC(=O)CCOCCN5C(=O)C=CC5=O)C(C)C)cc4)CC3)cnc2[C@H](C)OC)c2c3cc(ccc31)-c1cc(O)cc(c1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2]

1-(4-((S)-2-((S)-2-((tert-butoxycarbonyl)amino)-3-methylbutanamido)-5-ureidopentanamido)benzyl)-4-(5-((6³S,4S)-1¹-ethyl-2⁵-hydroxy-10,10-dimethyl-4-((S)-3-methyl-2-((2R,3R)—N-methyl-3-phenyltetrahydrofuran-2-carboxamido)butanamido)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)-1-methylpiperazin-1-ium trifluoroacetate (175 mg, 109 μmol) was treated with 8 mL of 25% TFA/CH2Cl2 with 0.1% triethylsilane for 1 hour at which time the volatiles were removed in vacuo. The residue was triturated with diethyl ether. The residue was dissolved in NMP (2 mL) and DIEA (133 μL, 765 μmol) and 2,5-dioxopyrrolidin-1-yl 3-(2-(2,5-dioxo-2,5-dihydro-1H-pyrrol-1-yl)ethoxy)propanoate (51 mg, 164 μmol) were added. After stirring for 2 hours, the solution was diluted with DMSO and purified by ISCO RP-HPLC. After lyophilization, the material was further purified by LH-20 Sephadex size exclusion chromatography (1:1 CH2Cl2/MeOH eluant) to remove Bu4N+ salt. After removal of volatiles in vacuo, dissolution in MeCN/H2O and lyophilization, 1-(4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-4-(5-((6³S,4S)-11-ethyl-2⁵-hydroxy-10,10-dimethyl-4-((S)-3-methyl-2-((2R,3R)—N-methyl-3-phenyltetrahydrofuran-2-carboxamido)butanamido)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)-1-methylpiperazin-1-ium trifluoroacetate (123 mg, 66% yield) was obtained. HRMS: M+=1581.8400, Rt=2.11 min, 5 min acidic method. ¹H NMR (400 MHz, DMSO) δ 10.21 (s, 1H), 9.34-9.18 (m, OH), 9.05 (s, 1H), 8.51 (d, J=2.6 Hz, 1H), 8.39 (d, J=7.0 Hz, OH), 8.15 (d, J=7.2 Hz, 1H), 7.95-7.80 (m, 2H), 7.76 (d, J=8.2 Hz, 3H), 7.65-7.47 (m, 4H), 7.47-7.34 (m, 1H), 7.34-7.10 (m, 5H), 7.01 (s, 3H), 6.91-6.63 (m, 1H), 6.58 (s, 1H), 5.99 (t, J=5.9 Hz, 1H), 5.51-5.27 (m, 3H), 5.19 (dd, J=15.7, 9.4 Hz, 1H), 5.09 (d, J=7.7 Hz, 1H), 4.65 (s, 2H), 4.38 (q, J=7.3 Hz, 1H), 4.34-3.99 (m, 7H), 3.84 (dtd, J=31.9, 15.7, 7.5 Hz, 5H), 3.71-3.40 (m, 15H), 3.33 (s, 39H), 3.11 (s, 3H), 3.09-2.88 (m, 6H), 2.88-2.59 (m, 8H), 2.47-2.16 (m, 6H), 1.94 (dq, J=10.7, 5.5 Hz, 2H), 1.68 (tdd, J=31.6, 16.6, 7.5 Hz, 5H), 1.54-1.42 (m, 2H), 1.37 (dd, J=10.2, 6.0 Hz, 4H), 1.09-0.92 (m, 4H), 0.92-0.78 (m, 9H), 0.73 (d, J=6.7 Hz, 1H), 0.68 (d, J=6.5 Hz, 3H), 0.50 (s, 3H), −0.06 (d, J=6.5 Hz, 2H).

### Synthesis of 1-(2-(((((9H-fluoren-9-yl)methoxy)carbonyl)(methyl)amino)methyl)-4-((S)-2-((S)-2-((tert-butoxycarbonyl)amino)-3-methylbutanamido)-5-ureidopentanamido)benzyl)-4-(5-((6³S,4S)-1¹-ethyl-10,10-dimethyl-4-((S)-3-methyl-2-((2R,3R)—N-methyl-3-phenyltetrahydrofuran-2-carboxamido)butanamido)-5,7-dioxo-2⁵-((triisopropylsilyl)oxy)-6¹,6²,6³,6⁴,6⁵,6-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)-1-methylpiperazin-1-ium trifluoroacetate

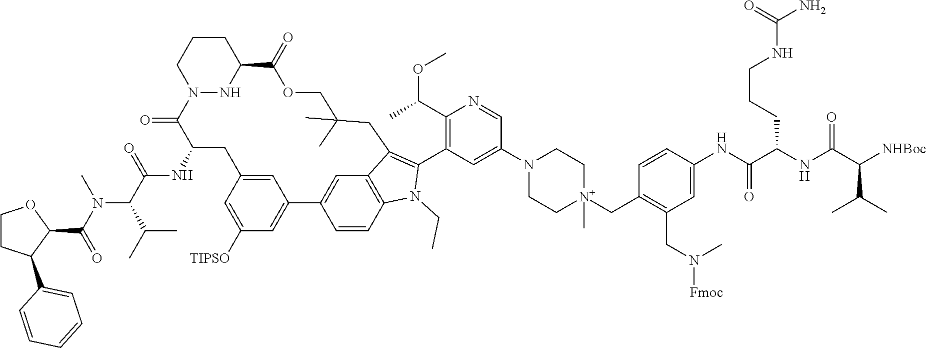

To a mixture of (2R,3R)—N-((2S)-1-(((6³S,4S)-1¹-ethyl-12-(2-((S)-1-methoxyethyl)-5-(4-methylpiperazin-1-yl)pyridin-3-yl)-10,10-dimethyl-5,7-dioxo-2⁵-((triisopropylsilyl)oxy-6¹,6²,6³,6⁴,6⁶-hexahydro-1′ H-8-oxa-1(5,3)-indola-6(1,3)-pyridazina-2(1,3)-benzenacycloundecaphane-4-yl)amino)-3-methyl-1-oxobutan-2-yl)-N-methyl-3-phenyltetrahydrofuran-2-carboxamide formate (250 mg, 0.193 mmol), (9H-fluoren-9-yl)methyl (5-((S)-2-((S)-2-((tert-butoxycarbonyl)amino)~3-methylbutanamido)-5-ureidopentanamido)-2-(chloromethyl)benzyl)(methyl)carbamate (177 mg, 0.232 mmol) and sodium iodide (28.9 mg, 0.193 mmol) in DMSO (2.5 mL) was added DIEA (67 μL, 0.386 mmol). After stirring for 5 hours the solution was diluted with DMSO (3 mL) and was directly purified by ISCO gold RP-HPLC. After lyophilization, 1-(2-(((((9H-fluoren-9-yl)methoxy)carbonyl)(methyl)amino)methyl)-4-((S)-2-((S)-2-((tert-butoxycarbonyl)amino)-3-methylbutanamido)-5-ureidopentanamido)benzyl)-4-(5-((6³S,4S)-1¹-ethyl-10,10-dimethyl-4-((S)-3-methyl-2-((2R,3R)—N-methyl-3-phenyltetrahydrofuran-2-carboxamido)butanamido)-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)-1-methylpiperazin-1-ium trifluoroacetate was obtained (379 mg, 96% yield). HRMS: M+=1908.1000, Rt=3.38 min, 5 min acidic method.

### Synthesis of 1-(4-((S)-2-((S)-2-((tert-butoxycarbonyl)amino)-3-methylbutanamido)-5-ureidopentanamido)-2-((methylamino)methyl)benzyl)-4-(5-((6³S,4S)-1¹-ethyl-2⁵-hydroxy-10,10-dimethyl-4-((S)-3-methyl-2-((2R,3R)—N-methyl-3-phenyltetrahydrofuran-2-carboxamido)butanamido)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)-1-methylpiperazin-1-ium trifluoroacetate

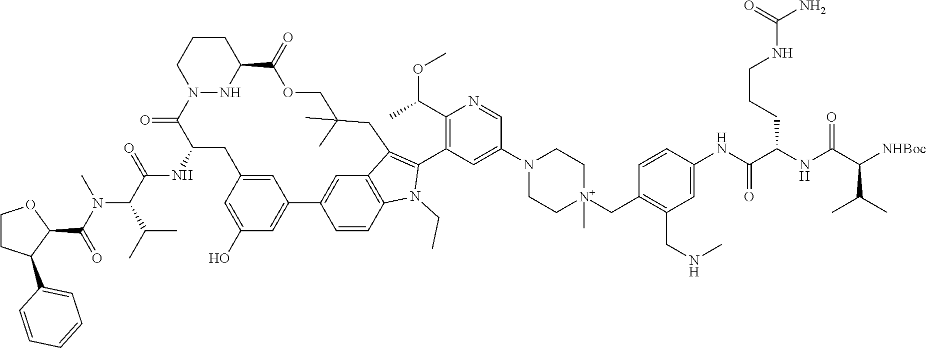 [SMILES: CCn1c(-c2cc(N3CC[N+](C)(Cc4ccc(NC(=O)[C@H](CCCNC(N)=O)NC(=O)[C@@H](NC(=O)OC(C)(C)C)C(C)C)cc4CNC)CC3)cnc2[C@H](C)OC)c2c3cc(ccc31)-c1cc(O)cc(c1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2]

To a solution of 1-(2-(((((9H-fluoren-9-yl)methoxy)carbonyl)(methyl)amino)methyl)-4-((S)-2-((S)-2-((tert-butoxycarbonyl)amino)-3-methylbutanamido)-5-ureidopentanamido)benzyl)-4-(5-((6³S,4S)-1¹-ethyl-10,10-dimethyl-4-((S)-3-methyl-2-((2R,3R)—N-methyl-3-phenyltetrahydrofuran-2-carboxamido)butanamido)-5,7-dioxo-2⁵-((triisopropylsilyl)oxy)-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)-1-methylpiperazin-1-ium trifluoroacetate (379 mg, 194 μmol) in NMP (3 mL) was added 1.0 TBAF in THF (210 μL, 210 μmol). After stirring for 45 minutes, piperidine (230 μL, 2.33 mmol) was added. After stirring for 30 minutes the solution was diluted with DMSO (9 ml) and was directly purified by ISCO gold RP-HPLC. After lyophilization, the material was further purified by LH-20 Sephadex size exclusion chromatography (1:1 CH2Cl2/MeOH eluant) to remove Bu4N+ salt. After removal of volatiles in vacuo, dissolution in MeCN/H2O and lyophilization, 1-(4-((S)-2-((S)-2-((tert-butoxycarbonyl)amino)-3-methylbutanamido)-5-ureidopentanamido)-2-((methylamino)methyl)benzyl)-4-(5-((6³S,4S)-1¹-ethyl-2⁵-hydroxy-10,10-dimethyl-4-((S)-3-methyl-2-((2R,3R)—N-methyl-3-phenyltetrahydrofuran-2-carboxamido)butanamido)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)-1-methylpiperazin-1-ium trifluoroacetate (235 mg, 68% yield) was obtained. HRMS: M+=1529.9000, Rt=2.02 min, 5 min acidic method.

### Synthesis of 1-(4-((S)-2-((S)-2-((tert-butoxycarbonyl)amino)-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(5-((6³S,4S)-1¹-ethyl-2⁵-hydroxy-10,10-dimethyl-4-((S)-3-methyl-2-((2R,3R)—N-methyl-3-phenyltetrahydrofuran-2-carboxamido)butanamido)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)-1-methylpiperazin-1-ium trifluoroacetate

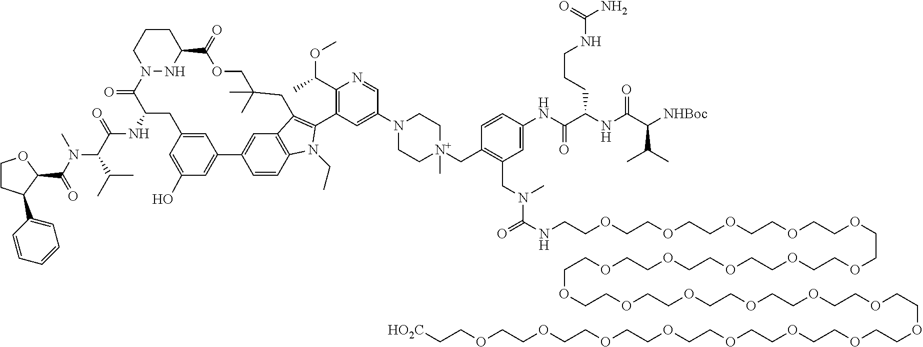 [SMILES: CCn1c(-c2cc(N3CC[N+](C)(Cc4ccc(NC(=O)[C@H](CCCNC(N)=O)NC(=O)[C@@H](NC(=O)OC(C)(C)C)C(C)C)cc4CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)CC3)cnc2[C@H](C)OC)c2c3cc(ccc31)-c1cc(O)cc(c1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2]

To a solution of tert-butyl 1-amino-3,6,9,12,15,18,21,24,27,30,33,36,39,42,45,48,51,54,57,60,63,66,69,72-tetracosaoxapentaheptacontan-75-oate (222 mg, 194 μmol) and DIEA (105 μL, 601 umol) in NMP (1.5 mL) was added of bis(4-nitrophenyl) carbonate (54.9 mg, 180 μmol). After stirring for 2 hours the activated amine solution was added to a solution of 1-(4-((S)-2-((S)-2-((tert-butoxycarbonyl)amino)-3-methylbutanamido)-5-ureidopentanamido)-2-((methylamino)methyl)benzyl)-4-(5-((6³S,4S)-11-ethyl-2⁵-hydroxy-10,10-dimethyl-4-((S)-3-methyl-2-((2R,3R)—N-methyl-3-phenyltetrahydrofuran-2-carboxamido)butanamido)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)-1-methylpiperazin-1-ium trifluoroacetate (235 mg, 133.6 μmol) and DIEA (140 μL, 802 μmol) in NMP (1 mL). The resulting solution was stirred for 30 minutes, was diluted with DMSO and purified by ISCO RP-HPLC. Upon lyophilization, 1-(4-((S)-2-((S)-2-((tert-butoxycarbonyl)amino)-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(5-((6³S,4S)-11-ethyl-2⁵-hydroxy-10,10-dimethyl-4-((S)-3-methyl-2-((2R,3R)—N-methyl-3-phenyltetrahydrofuran-2-carboxamido)butanamido)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)-1-methylpiperazin-1-ium trifluoroacetate (291 mg, 77% yield) was obtained. HRMS: M+=2701.5600, Rt=2.30 min (5 min acidic method).

### Synthesis of 1-(2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-4-(5-((6³S,4S)-1¹-ethyl-2⁵-hydroxy-10,10-dimethyl-4-((S)-3-methyl-2-((2R,3R)—N-methyl-3-phenyltetrahydrofuran-2-carboxamido)butanamido)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)-1-methylpiperazin-1-ium trifluoroacetate (L101-D101)

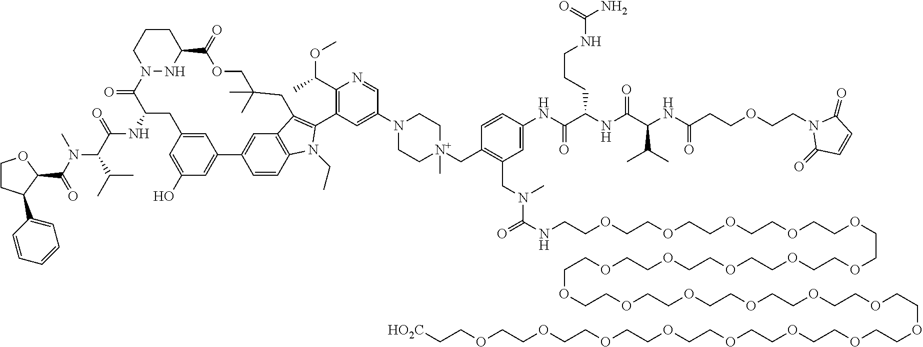 [SMILES: CCn1c(-c2cc(N3CC[N+](C)(Cc4ccc(NC(=O)[C@H](CCCNC(N)=O)NC(=O)[C@@H](NC(=O)CCOCCN5C(=O)C=CC5=O)C(C)C)cc4CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)CC3)cnc2[C@H](C)OC)c2c3cc(ccc31)-c1cc(O)cc(c1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2]

1-(4-((S)-2-((S)-2-((tert-butoxycarbonyl)amino)-3-methylbutanamido)-5-ureidopentanamido)-2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)benzyl)-4-(5-((6³S,4S)-11-ethyl-2⁵-hydroxy-10,10-dimethyl-4-((S)-3-methyl-2-((2R,3R)—N-methyl-3-phenyltetrahydrofuran-2-carboxamido)butanamido)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)-1-methylpiperazin-1-ium trifluoroacetate (356 mg, 126.4 μmol) was treated with 15 mL of 25% TFA/CH2Cl2 with 0.1% Triethylsilane for 1 hour at which time the volatiles were removed in vacuo. The residue was triturated with diethyl ether. The residue was dissolved in NMP (4 mL) and DIEA (352 μL, 2022 μmol) and 2,5-dioxopyrrolidin-1-yl 3-(2-(2,5-dioxo-2,5-dihydro-1H-pyrrol-1-yl)ethoxy)propanoate (55 mg, 177 μmol) were added. After stirring for 2 hours the solution was diluted with DMSO and purified by ISCO RP-HPLC. Upon lyophilization, 1-(2-(78-carboxy-2-methyl-3-oxo-7,10,13,16,19,22,25,28,31,34,37,40,43,46,49,52,55,58,61,64,67,70,73,76-tetracosaoxa-2,4-diazaoctaheptacontyl)-4-((S)-2-((S)-2-(3-(2-(2,5-dioxo-2,5-dihydro-1H-pyrrol-1-yl)ethoxy)propanamido)-3-methylbutanamido)-5-ureidopentanamido)benzyl)-4-(5-((6³S,4S)-1¹-ethyl-2⁵-hydroxy-10,10-dimethyl-4-((S)-3-methyl-2-((2R,3R)—N-methyl-3-phenyltetrahydrofuran-2-carboxamido)butanamido)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-1²-yl)-6-((S)-1-methoxyethyl)pyridin-3-yl)-1-methylpiperazin-1-ium trifluoroacetate (300 mg, 81% yield) was obtained. HRMS: M+=2796.5701, Rt=2.20 min (5 min acidic method). ¹H NMR (400 MHz, DMSO) δ 12.15 (s, 1H), 10.25 (s, 1H), 9.21 (s, OH), 9.04 (s, 1H), 8.50 (d, J=2.6 Hz, 1H), 8.38 (d, J=6.9 Hz, OH), 8.13 (d, J=7.1 Hz, 1H), 7.96-7.66 (m, 3H), 7.64-7.50 (m, 1H), 7.50-7.32 (m, 2H), 7.32-7.10 (m, 4H), 7.00 (s, 2H), 6.92-6.61 (m, 1H), 6.61-6.44 (m, 1H), 5.99 (t, J=5.9 Hz, 1H), 5.57-5.14 (m, 3H), 5.09 (d, J=7.7 Hz, 1H), 4.73 (s, 1H), 4.60 (s, 1H), 4.46-4.32 (m, 1H), 4.32-4.00 (m, 5H), 3.84 (dtd, J=32.1, 15.8, 7.5 Hz, 4H), 3.71-3.63 (m, 2H), 3.59 (q, J=6.4 Hz, 4H), 3.55-3.45 (m, 78H), 3.41 (d, J=6.5 Hz, 21H), 3.22 (q, J=6.1 Hz, 2H), 3.17-2.99 (m, 6H), 2.93 (q, J=6.1 Hz, 1H), 2.76 (s, 4H), 2.66 (s, 3H), 2.43 (t, J=6.3 Hz, 3H), 2.38-2.19 (m, 3H), 1.94 (dq, J=13.6, 7.2 Hz, 2H), 1.84-1.43 (m, 5H), 1.36 (dd, J=10.1, 6.1 Hz, 4H), 1.05-0.89 (m, 3H), 0.89-0.77 (m, 6H), 0.77-0.59 (m, 3H), 0.50 (s, 2H), −0.06 (d, J=6.5 Hz, 2H).

### Synthesis of (6³S,4S)-1¹-ethyl-1²-(2-((S)-1-methoxyethyl)-5-(4-methylpiperazin-1-yl)pyridin-3-yl)-10,10-dimethyl-4-((S)-3-methyl-2-((2R,3R)—N-methyl-3-phenyltetrahydrofuran-2-carboxamido)butanamido)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl (4-nitrophenyl) carbonate trifluoroacetate

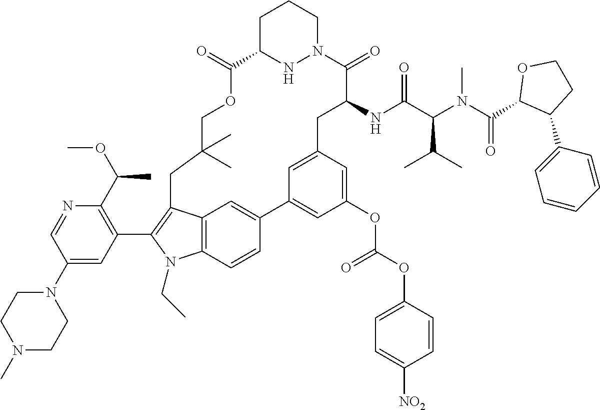 [SMILES: CCn1c(-c2cc(N3CCN(C)CC3)cnc2[C@H](C)OC)c2c3cc(ccc31)-c1cc(cc(OC(=O)Oc3ccc([N+](=O)[O-])cc3)c1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2]

(2R,3R)—N-((2S)-1¹-(((6³S,4S)-1¹-ethyl-12-(2-((S)-1-methoxyethyl)-5-(4-methylpiperazin-1-yl)pyridin-3-yl)~10,10-dimethyl-5,7-dioxo-2~((triisopropylsilyl)oxy)-6¹,6²,6³,6⁴,6⁵,6⁶-hexahydro-1¹H-8-oxa-1(5,3)-indola-6(1,3)-pyridazina-2(1,3-benzenacycoundecaphane-4-yl)amino)-3-methyl-1-oxobutan-2-yl)-N-methyl-3-phenyltetrahydrofuran-2-carboxamide formate (37 mg, 35 μmol) in a 4 mL vial open to air at 23° C., was treated with DMF (1.0 mL) followed by DIPEA (12.0 μL, 69 μmol) and lastly bis(4-nitrophenyl) carbonate (21 mg, 69 μmol). The reaction was stirred for a total of 2 hrs, upon which time it was diluted with a total of 2 mL DMSO and loaded onto a 50 g C18 column. The material was purified via a CH₃CN and water gradient with 0.1% TFA modifier (20%-80% CH₃CN). Desired fractions were combined, frozen, and lyophilized to yield (6³S,4S)-1¹-ethyl-1²-(2-((S)-1-methoxyethyl)-5-(4-methylpiperazin-1-yl)pyridin-3-yl)-10,10-dimethyl-4-((S)-3-methyl-2-((2R,3R)—N-methyl-3-phenyltetrahydrofuran-2-carboxamido)butanamido)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl (4-nitrophenyl) carbonate trifluoroacetate (39 mg, 85% yield) as a yellow powder. HRMS: M+=1212.5699, Rt=2.43 min (5 min acidic method).

### Synthesis of 4-((S)-2-((S)-2-((tert-butoxycarbonyl)amino)-3-methylbutanamido)-5-ureidopentanamido)-2-((methylamino)methyl)benzyl ((6³S,4S)-1¹-ethyl-1²-(2-((S)-1-methoxyethyl)-5-(4-methylpiperazin-1-yl)pyridin-3-yl)-10,10-dimethyl-4-((S)-3-methyl-2-((2R,3R)—N-methyl-3-phenyltetrahydrofuran-2-carboxamido)butanamido)-5,7-dioxo-6¹,6²,6³,6⁴,6⁵,6⁶-hexahydro-11H-8-oxa-1(5,3)-indola-6(1,3)-pyridazina-2(1,3)-benzenacycloundecaphane-2⁵-yl) ethane-1,2-diylbis(methylcarbamate) trifluoroacetate

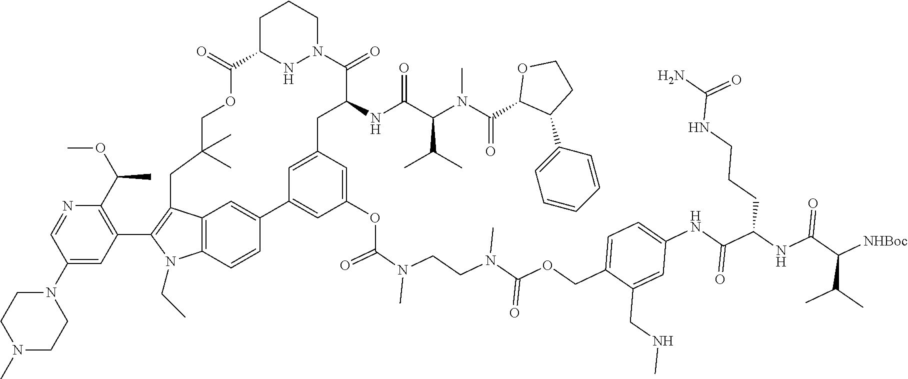 [SMILES: CCn1c(-c2cc(N3CCN(C)CC3)cnc2[C@H](C)OC)c2c3cc(ccc31)-c1cc(cc(OC(=O)N(C)CCN(C)C(=O)OCc3ccc(NC(=O)[C@H](CCCNC(N)=O)NC(=O)[C@@H](NC(=O)OC(C)(C)C)C(C)C)cc3CNC)c1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2]

(6³S,4S)-1¹-ethyl-1²-(2-((S)-1-methoxyethyl)-5-(4-methylpiperazin-1-yl)pyridin-3-yl)-10,10-dimethyl-4-((S)-3-methyl-2-((2R,3R)—N-methyl-3-phenyltetrahydrofuran-2-carboxamido)butanamido)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl (4-nitrophenyl) carbonate trifluoroacetate (39 mg, 30 μmol) in a 4 mL vial open to air at 23° C., was treated with 2-(((((9H-fluoren-9-yl)methoxy)carbonyl)(methyl)amino)methyl)-4-((S)-2-((S)-2-((tert-butoxycarbonyl)amino)-3-methylbutanamido)-5-ureidopentanamido)benzyl methyl(2-(methylamino)ethyl)carbamate trifluoroacetate (34 mg, 35 μmol) followed by DMF (0.60 mL). Next, DIPEA (31 μL, 180 μmol) was added. The reaction was stirred at 23° C. for 1 hr. To this mixture, dimethylamine in THF (150 μL, 300 μmol) was added. The reaction was stirred at 23° C. for 1.75 hr, upon which time it was diluted with 2 mL DMSO and loaded onto a 50 g C18 column. The material was purified via a CH₃CN and water gradient with 0.1% TFA modifier (15%-60% CH₃CN, 0.1% TFA). Desired fractions were combined, frozen, and lyophilized to yield 4-((S)-2-((S)-2-((tert-butoxycarbonyl)amino)-3-methylbutanamido)-5-ureidopentanamido)-2-((methylamino)methyl)benzyl ((6³S,4S)-1¹-ethyl-1²-(2-((S)-1-methoxyethyl)-5-(4-methylpiperazin-1-yl)pyridin-3-yl)-10,10-dimethyl-4-((S)-3-methyl-2-((2R,3R)—N-methyl-3-phenyltetrahydrofuran-2-carboxamido)butanamido)-5,7-dioxo-6¹,6²,6³,6⁴,6⁵,6⁶-hexahydro-11H-8-oxa-1(5,3)-indola-6(1,3)-pyridazina-2(1,3)-benzenacycloundecaphane-2⁵-yl) ethane-1,2-diylbis(methylcarbamate) trifluoroacetate (37 mg, 70% yield) as a yellow powder. HRMS: M+=1687.9700, Rt=1.95 min (5 min acidic method).

### Synthesis of 1-(5-((S)-2-((S)-2-((tert-butoxycarbonyl)amino)-3-methylbutanamido)-5-ureidopentanamido)-2-((((2-(((((6³S,4S)-1¹-ethyl-1²-(2-((S)-1-methoxyethyl)-5-(4-methylpiperazin-1-yl)pyridin-3-yl)-10,10-dimethyl-4-((S)-3-methyl-2-((2R,3R)—N-methyl-3-phenyltetrahydrofuran-2-carboxamido)butanamido)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)oxy)carbonyl)(methyl)amino)ethyl)(methyl)carbamoyl)oxy)methyl)phenyl)-2-methyl-3-oxo-7,10,13,16,19,22,25,28,31,34,37,40,43,46,49,52,55,58,61,64,67,70,73,76-tetracosaoxa-2,4-diazanonaheptacontan-79-oic acid trifluoroacetate

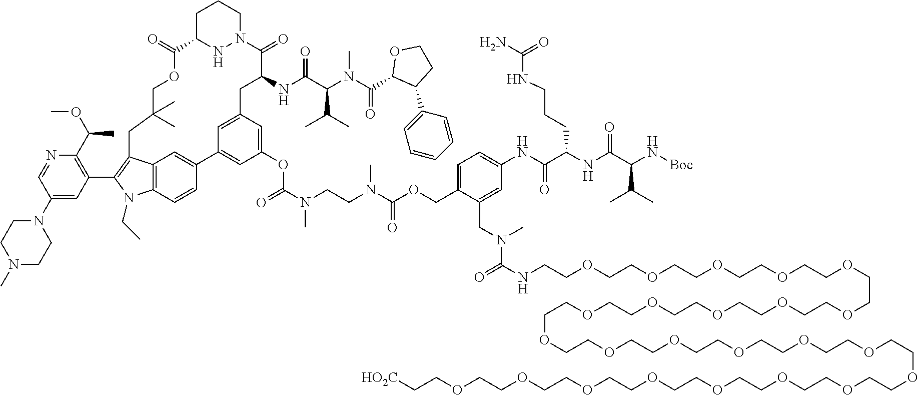 [SMILES: CCn1c(-c2cc(N3CCN(C)CC3)cnc2[C@H](C)OC)c2c3cc(ccc31)-c1cc(cc(OC(=O)N(C)CCN(C)C(=O)OCc3ccc(NC(=O)[C@H](CCCNC(N)=O)NC(=O)[C@@H](NC(=O)OC(C)(C)C)C(C)C)cc3CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)c1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2]

1-amino-3,6,9,12,15,18,21,24,27,30,33,36,39,42,45,48,51,54,57,60,63,66,69,72-tetracosaoxapentaheptacontan-75-oic acid (35 mg, 30 μmol) in a 4 mL vial open to air at 23° C., was treated with DMF (0.50 mL). This mixture was stirred for 1-2 min., until all the material had dissolved. Next, DIPEA (22 μL, 120 μmol) was added followed by bis(4-nitrophenyl) carbonate (8.5 mg, 28 μmol). The reaction was stirred at 23° C. for 30 min, upon which time additional DIPEA (11 μL, 62 μmol) was added and this reaction was added to 4-((S)-2-((S)-2-((tert-butoxycarbonyl)amino)-3-methylbutanamido)-5-ureidopentanamido)-2-((methylamino)methyl)benzyl((6³S,4S)-1¹-ethyl-1²-(2-((S)-1-methoxyethyl)-5-(4-methylpiperazin-1-yl)pyridin-3-yl)-10,10-dimethyl-4-((S)-3-methyl-2-((2R,3R)—N-methyl-3-phenyltetrahydrofuran-2-carboxamido)butanamido)-5,7-dioxo-6¹,6²,6³,6⁴,6⁵,6⁶-hexahydro-11H-8-oxa-1(5,3)-indola-6(1,3)-pyridazina-2(1,3)-benzenacycloundecaphane-2⁵-yl) ethane-1,2-diylbis(methylcarbamate) trifluoroacetate (37 mg, 21 μmol) in a 4 mL vial exposed to air at 23° C. The reaction was stirred at 23° C. for 45 min., upon which time it was diluted to a total volume of 3 mL with DMSO and loaded onto a 50 g C18 column. The material was purified via a CH₃CN and water gradient with 0.1% TFA modifier (15%-80% CH₃CN, 0.1% TFA). Desired fractions were combined, frozen and lyophilized to yield 1-(5-((S)-2-((S)-2-((tert-butoxycarbonyl)amino)-3-methylbutanamido)-5-ureidopentanamido)-2-((((2-(((((6³S,4S)-1¹-ethyl-1²-(2-((S)-1-methoxyethyl)-5-(4-methylpiperazin-1-yl)pyridin-3-yl)-10,10-dimethyl-4-((S)-3-methyl-2-((2R,3R)—N-methyl-3-phenyltetrahydrofuran-2-carboxamido)butanamido)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)oxy)carbonyl)(methyl)amino)ethyl)(methyl)carbamoyl)oxy)methyl)phenyl)-2-methyl-3-oxo-7,10,13,16,19,22,25,28,31,34,37,40,43,46,49,52,55,58,61,64,67,70,73,76-tetracosaoxa-2,4-diazanonaheptacontan-79-oic acid trifluoroacetate (22 mg, 35% yield) as a yellow powder. HRMS: M+2H+=1430.8101, Rt=2.23 min (5 min acidic method). ¹H NMR (400 MHz, DMSO) δ 12.13 (s, 1H), 10.11 (s, 1H), 9.63 (s, 1H), 8.51 (d, J=2.9 Hz, 1H), 7.96 (d, J=7.8 Hz, 2H), 7.83-6.88 (m, 14H), 6.88-6.58 (m, 3H), 6.43 (s, 1H), 5.96 (s, 1H), 5.66-4.84 (m, 7H), 4.47 (d, J=24.7 Hz, 3H), 4.33-4.08 (m, 5H), 4.01 (t, J=9.8 Hz, 3H), 3.90 (s, 1H), 3.81 (q, J=8.9 Hz, 2H), 3.71 (d, J=6.7 Hz, 1H), 3.65 (d, J=13.0 Hz, 2H), 3.59 (t, J=6.4 Hz, 3H), 3.52-3.48 (m, 85H), 3.21-3.12 (m, 5H), 3.06 (d, J=4.6 Hz, 9H), 2.96-2.80 (m, 10H), 2.80-2.64 (m, 8H), 2.58 (d, J=6.2 Hz, 3H), 2.43-2.31 (m, 9H), 1.95 (d, J=15.7 Hz, 3H), 1.79 (s, 1H), 1.60 (d, J=44.3 Hz, 5H), 1.41-1.29 (m, 14H), 0.99 (d, J=6.8 Hz, 4H), 0.88-0.46 (m, 16H), −0.07 (dd, J=6.6, 3.0 Hz, 3H).

### Synthesis of 1-(5-((S)-2-((S)-2-(3-(2-(2,5-dioxo-2,5-dihydro-1H-pyrrol-1-yl)ethoxy)propanamido)-3-methylbutanamido)-5-ureidopentanamido)-2-((((2-(((((6³S,4S)-1¹-ethyl-1²-(2-((S)-1-methoxyethyl)-5-(4-methylpiperazin-1-yl)pyridin-3-yl)-10,10-dimethyl-4-((S)-3-methyl-2-((2R,3R)—N-methyl-3-phenyltetrahydrofuran-2-carboxamido)butanamido)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)oxy)carbonyl)(methyl)amino)ethyl)(methyl)carbamoyl)oxy)methyl)phenyl)-2-methyl-3-oxo-7,10,13,16,19,22,25,28,31,34,37,40,43,46,49,52,55,58,61,64,67,70,73,76-tetracosaoxa-2,4-diazanonaheptacontan-79-oic acid trifluoroacetate (L103-D101)

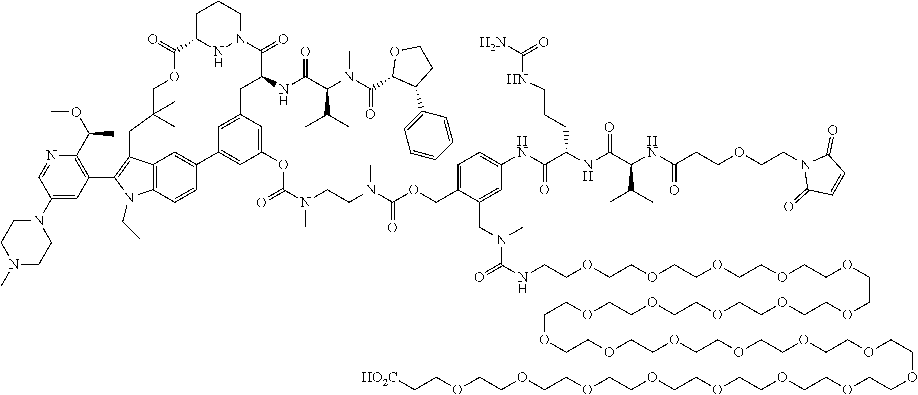 [SMILES: CCn1c(-c2cc(N3CCN(C)CC3)cnc2[C@H](C)OC)c2c3cc(ccc31)-c1cc(cc(OC(=O)N(C)CCN(C)C(=O)OCc3ccc(NC(=O)[C@H](CCCNC(N)=O)NC(=O)[C@@H](NC(=O)CCOCCN4C(=O)C=CC4=O)C(C)C)cc3CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O)c1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2]

1-(5-((S)-2-((S)-2-((tert-butoxycarbonyl)amino)-3-methylbutanamido)-5-ureidopentanamido)-2-((((2-(((((6³S,4S)-1¹-ethyl-1²-(2-((S)-1-methoxyethyl)-5-(4-methylpiperazin-1-yl)pyridin-3-yl)-10,10-dimethyl-4-((S)-3-methyl-2-((2R,3R)—N-methyl-3-phenyltetrahydrofuran-2-carboxamido)butanamido)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)oxy)carbonyl)(methyl)amino)ethyl)(methyl)carbamoyl)oxy)methyl)phenyl)-2-methyl-3-oxo-7,10,13,16,19,22,25,28,31,34,37,40,43,46,49,52,55,58,61,64,67,70,73,76-tetracosaoxa-2,4-diazanonaheptacontan-79-oic acid trifluoroacetate (22 mg, 7.4 μmol) in a 4 mL vial open to air at 23° C., was treated with 25% TFA/DCM, 0.1% Et₃SiH (1.0 mL). The reaction was stirred at 23° C. for 1 hr, upon which time it was transferred to a separate 4 mL vial washing with 2 mL DCM, and the mixture was concentrated under reduced pressure (RT water bath). The resulting oil was triturated 2×3 mL Et₂O, and the Et₂O was decanted. The vial was placed on under reduced pressure, which produced a yellow solid. The solid was dissolved in NMP (0.50 mL) and DIPEA (19 μL, 110 μmol) was added. Next, 2,5-dioxopyrrolidin-1-yl 3-(2-(2,5-dioxo-2,5-dihydro-1H-pyrrol-1-yl)ethoxy)propanoate (3.2 mg, 10 μmol) was added in a single portion and the reaction was stirred at 23° C. for 2 hrs, upon which time it was diluted with DMSO (2.5 mL) and loaded onto a 50 g C18 column. The material was purified via a CH₃CN and water gradient with 0.1% TFA modifier (20%-80% CH₃CN, 0.1% TFA). Desired fractions were combined, frozen, and lyophilized to yield 1-(5-((S)-2-((S)-2-(3-(2-(2,5-dioxo-2,5-dihydro-1H-pyrrol-1-yl)ethoxy)propanamido)-3-methylbutanamido)-5-ureidopentanamido)-2-((((2-(((((6³S,4S)-1¹-ethyl-1²-(2-((S)-1-methoxyethyl)-5-(4-methylpiperazin-1-yl)pyridin-3-yl)-10,10-dimethyl-4-((S)-3-methyl-2-((2R,3R)—N-methyl-3-phenyltetrahydrofuran-2-carboxamido)butanamido)-5,7-dioxo-6¹,6²,6³,6⁴,6⁵,6⁶-hexahydro-1¹H-8-oxa-1(5,3)-indola-6(1,3)-pyridazina-2(1,3)-benzenacycloundecaphane-2⁵-yl)oxy)carbonyl)(methyl)amino)ethyl)(methyl)carbamoyl)oxy)methyl)phenyl)-2-methyl-3-oxo-7,10,13,16,19,22,25,28,31,34,37,40,43,46,49,52,55,58,61,64,67,70,73,76-tetracosaoxa-2,4-diazanonaheptacontan-79-oic acid trifluoroacetate (10 mg, 45% yield) as an off-white solid. HRMS: M+2H+=1478.3101, Rt=2.12 min (5 min acidic method). ¹H NMR (400 MHz, DMSO) δ 12.10 (s, OH), 10.03 (s, 1H), 9.66 (s, 1H), 8.51 (d, J=3.0 Hz, 1H), 8.16-7.94 (m, 2H), 7.83 (d, J=8.6 Hz, 1H), 7.79-7.33 (m, 7H), 7.33-7.09 (m, 7H), 7.00 (s, 2H), 6.86-6.66 (m, 2H), 6.43 (s, 1H), 5.99 (s, 1H), 5.72-4.88 (m, 6H), 4.70-3.98 (m, 12H), 3.96-3.57 (m, 19H), 3.50 (s, 76H), 3.27-2.64 (m, 36H), 2.58 (d, J=6.4 Hz, 5H), 2.43 (q, J=5.6 Hz, 6H), 2.34-2.18 (m, 4H), 2.09-1.86 (m, 3H), 1.81 (s, 1H), 1.75-1.49 (m, 5H), 1.37 (dd, J=9.9, 6.1 Hz, 6H), 0.99 (d, J=6.9 Hz, 4H), 0.92-0.51 (m, 16H), −0.07 (dd, J=6.6, 3.0 Hz,

### Example 4. Conjugation and Characterization of ADCs

### In Vitro Cysmab ADC Preparation

Antibody (typically 5-10 mg) was incubated with rProtein A Sepharose resin (GE) at a ratio of 10 mg Ab to 1 ml resin in PBS for 15 minutes with mixing in an appropriately sized disposable column. Cysteine HCl was added to a final concentration of 20 mM and incubated with agitation for 30 min at room temperature to allow the reactive cysteines to be deblocked. The resin was rapidly washed with 50 column volumes PBS on a vacuum manifold in multiple additions. The resin was then resuspended in an equal volume PBS containing 250 nM CuCl₂. Reformation of antibody interchain disulfides was monitored by taking time points. At each time point, 25 μL of resin slurry was removed, 1 μL of 20 mM MC-valcit-MMAE was added, and the tube flicked several times. The resin was spun down, supernatant removed, and then eluted with 50 μL Antibody elution buffer (Thermo). The resin was pelleted and the supernatant analyzed by reverse phase chromatography using an Agilent PLRP-S 4000A 5 μm, 4.6×50 mm column (Buffer A is water, 0.1% TFA, Buffer B Acetonitrile, 0.1% TFA, column held at 80° C., Flowrate 1.5 ml/min; Gradient 0 minutes—30% B, 5 minutes—45% B, 6.5 min—100% B, 8 minutes—100% B, 10 minutes—30%).

Once determined that the antibody has reformed its interchain disulfide bonds, the resin was washed with 10 column volumes PBS and the resin was resuspended in an equal volume PBS and 12 equivalents of the appropriate linker-payload (20 mM) in DMSO was added and then incubated at room temperature for 2 hours. The resin was then washed with 50 column volumes PBS to remove excess linker-payload. The ADC was eluted from the protein A resin with antibody elution buffer. The ADC was then dialyzed into PBS. The material was then concentrated using a centrifugal concentrator using an Amicon Ultra-15, 50KDa, regenerated cellulose (Millipore, UFC0905024), to 4.5 mg/ml and filtered sterilely through 0.22 μm sterile PVDF Filter, 25 mm (Millapore, SLGV013SL) and stored at 4° C. The following analyses were performed—analytical SEC to determine percent monomer, reduced mass spectroscopy to determine DAR, LAL test to determine endotoxin load and protein concentration was determined by A280 utilizing extinction coefficient and molecular weight of antibody. All in vitro materials were >90% monomer. Percent aggregation, as determined by comparison of the area of the high-molecular-weight peak absorbance at 210 and 280 nm with the area of the peak absorbance for monomeric ADC. HRMS data (protein method) indicated a dominant mass of the heavy chain+2 species, giving a DAR of ~4.0 was calculated by comparing MS intensities of peaks for DAR1 DAR2 and DAR3 species.

General Methodology: Drug-to-antibody ratio (DAR) of exemplary ADCs was determined by liquid chromatography-mass spectrometry (LC/MS) according to the following method. For all LC methods, mobile phase A was purified MS grade water (Honeywell, LC015-1), mobile phase B was MS grade 80% Isopropanol (Honeywell LC323-1): 20% acetonitrile (Honeywell, LC015-1), LC323-1), supplemented with 1% of formic acid (FA) (Thermo Scientific, 85178). The column temperature was set at 80° C. A general MS method was optimized for all ADCs synthesized. The column used for analysis was an Agilent PLRP-S 4000 A; 2.1×150 mm, 8 um (Agilent, PL1912-3803). Flowrate used was 0.3 ml/min. The gradient used was 0-0.75 minute 95% A, 0.76-1.9 minute 75% A, 1.91-11.0 minute 50% A, 11.01-11.50 10% A, 11.51-13.50 minute 95% A, 13.51-18 minute 95% A on an Acuity Bio H-Class Quaternary UPLC (Waters). MS system was Xevo G2-XS QToF ESI mass spectrometer (Waters) and data acquired from 1.5-11 minutes and masses were analyzed between 15000-80000 daltons. DAR was determined from the deconvoluted spectra or UV chromatogram by summing the integrated MS (total ion current) or UV (280 nm) peak area of unconjugated and conjugated given species (mAb or associated fragment), weighted by multiplying each area by the number of drug attached. The summed, weighted areas were divided by the sum of total area and the results produced a final average DAR value for the full ADC.

Size exclusion chromatography (SEC): SEC was performed to determine the quality of the ADCs and aggregation percentage (%) after purification. The analysis was performed on analytical column Superdex 200 Increase 5/150 GL (GE Healthcare, 28990945) in isocratic conditions 100% PBS pH 7.2 ((Hyclone SH30028.03)), flow 0.45 ml/min for 8 minutes. The % aggregate fraction of the ADC sample was quantified based on the peak area absorbance at 280 nm. Calculation was based on the ratio between the high molecular weight eluent at 280 nm divided by the sum of peak area absorbance at the same wavelength of the high molecular weight and monomeric eluents multiplied by 100%. Data was acquired on an Agilent Bio-Inert 1260 HPLC outfitted with a Wyatt miniDAWN light scattering and Treos refractive index detectors (Wyatt Technologies, Santa Barbara, CA).

### Example 5. In Vitro Assessment of panRAS Antibody Drug Conjugates in Multiple Cancer Cell Line

The panRAS antibody drug conjugates were tested against the following cancer cell lines:
LU65: JCRB No. JCRB0079 cultured in RPMI-1640+10% FBS
HPAC: ATCC No. CRL-2119 cultured in DMEM:HAM's F12+5% FBS
NCI-H727: ATCC No. CRL-5815 cultured in RPMI-1640+10% FBS
SW1271: ATCC No. CRL-2177 cultured in DMEM+10% FBS

### Inhibition of Cell Proliferation and Survival

The ability of the panRAS antibody drug conjugates to inhibit cell proliferation and survival was assessed using the Promega CellTiter-Glo® proliferation assay.

Cell lines were cultured in media that is optimal for their growth at 5% CO₂, 37° C. in a tissue culture incubator. Prior to seeding for the proliferation assay, the cells were split at least 2 days before the assay to ensure optimal growth density. On the day of seeding, cell viability and cell density were determined using a cell counter (Vi-Cell XR Cell Viability Analyzer, Beckman Coulter). Cells with higher than 85% viability were seeded in white clear bottom 384-well TC treated plates (Corning cat. #3765). Cells were seeded at a density of 1,000 cells per well in 45 μL of standard growth media. Plates were incubated at 5% CO₂, 37° C. overnight in a tissue culture incubator. The next day, indicated compounds and ADCs were prepared at 10× in standard growth media. The prepared compounds and ADCs were then added to the cells resulting in final concentrations of 0.005-100 nM and a final volume of 50 μL per well. Each drug concentration was tested in quadruplets. Plates were incubated at 5% CO₂, 37° C. for 5 days in a tissue culture incubator, after which cell viability was assessed through the addition of 25 μL of CellTiter Glo® (Promega, cat #G7573), a reagent which lyses cells and measures total adenosine triphosphate (ATP) content. Plates were incubated at room temperature for 10 minutes to stabilize luminescent signals prior to reading using a luminescence reader (EnVision Multilabel Plate Reader, PerkinElmer). To evaluate the effect of the drug treatments, luminescent counts from wells containing untreated cells (100% viability) were used to normalize treated samples. A variable slope model was applied to fit a nonlinear regression curve to the data in GraphPad PRISM version 7.02 software. IC50 and Amax values were extrapolated from the resultant curves.

The dose response curves of representative cancer cell lines are shown in FIG. 1. The concentrations of treatment required to inhibit 50% of cell growth or survival (IC50) were calculated with representative IC50 values of the cell lines tested summarized in Table I. The panRAS ADCs tested (in the table below the “panRAS ADC” is Ab-101-D101) on representative cell lines demonstrated in vitro efficacy relative to the isotype matched non-targeting control ADC.

These studies indicate that panRAS ADCs were capable of inhibiting cell proliferation on various cancer cell lines expressing antigens of interest. No cytotoxic activity was observed by the isotype matched non-targeting controls on the cancer cell lines tested.

TABLE I
PanRAS ADC cytotoxicity

| Compound | Cell line | IC50 (nM) | Amax (Span) |
|---|---|---|---|
| CD276-panRas ADC | Lu65 | 0.0706 | 69.1 |
| Isotype ADC | Lu65 | 26.3 | 68.5 |
| Sotorasib | Lu65 | — | — |
| CD276-panRas ADC | H727 | 3.42 | 74.9 |
| Isotype ADC | H727 | >100 | — |
| Sotorasib | H727 | >100 | — |
| EPHA2-panRas ADC | HPAC | 0.400 | 65.1 |
| Isotype ADC | HPAC | >100 | — |
| Sotorasib | HPAC | >100 | — |
| EPHA2-panRas ADC | SW1271 | 0.368 | 53.9 |
| Isotype ADC | SW1271 | 29.5 | 64.3 |
| Sotorasib | SW1271 | >100 | — |

1. An antibody-drug conjugate of Formula (1): 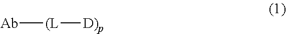
wherein Ab is an antibody or an antigen-binding fragment thereof;
L is a conjugate linker that covalently attaches Ab to D;
p is an integer from 1 to 16; and
D is a panRAS inhibitor.

2. The antibody-drug conjugate of claim 1, wherein p is an integer from 1 to 6 or from 2 to 4, or p is 2 or 4; or p is determined by liquid chromatography-mass spectrometry (LC-MS).

3. The antibody-drug conjugate of claim 1 or 2, wherein L comprises:
an attachment group;
at least one bridging spacer group; and
at least one cleavable group, optionally at least one cleavable group comprising a pyrophosphate group and/or a self-immolative group.

4. The antibody-drug conjugate of claim 3, wherein -(L-D) is of the formula (A): 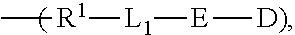
wherein:
R¹ is an attachment group;
L₁ is a bridging spacer group;
E is a cleavable group.

5. The antibody-drug conjugate of claim 3 or 4, wherein the cleavable group comprises a pyrophosphate group or the cleavable group comprises

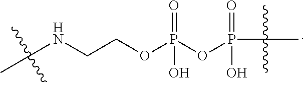 [SMILES: *NCCOP(=O)(O)OP(*)(=O)O]

6. The antibody-drug conjugate of claim 3 or 4, wherein the bridging spacer group comprises:
(i) a polyoxyethylene (PEG) group;
(ii) a PEG group selected from, PEG1, PEG2, PEG3, PEG4, PEG5, PEG6, PEG7, PEG8, PEG9, PEG10, PEG11, PEG12, PEG13, PEG14, and PEG15;
(iii) a —CO—CH₂—CH₂—PEG12- group;
(iv) a butanoyl, pentanoyl, hexanoyl, heptanoyl, or octanoyl group; or
(v) a hexanoyl group.

7. The antibody-drug conjugate of claim 6, wherein (i) the attachment group is formed from at least one reactive group selected from a maleimide group, thiol group, cyclooctyne group, and an azido group; optionally wherein:
a) the maleimide group has the structure: 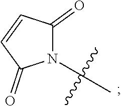 [SMILES: *N1C(=O)C=CC1=O]
b) the azido group has the structure: —N═N⁺═N⁻;
c) the cyclooctyne group has the structure: 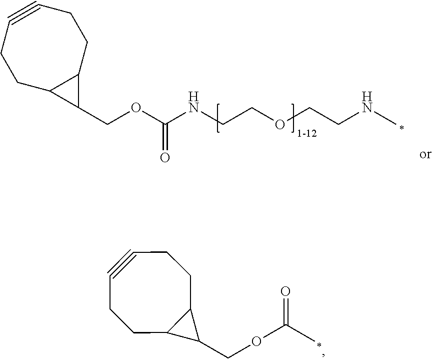
and wherein —* is a bond to the antibody or antigen-binding gradient thereof; or
d) the cyclooctyne group has the structure: 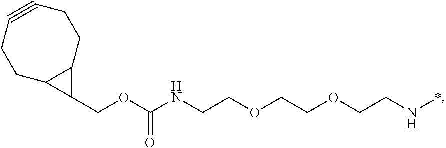
and wherein —* is a bond to the antibody or antigen-binding fragment thereof; or
(ii) the attachment group has a formula comprising: 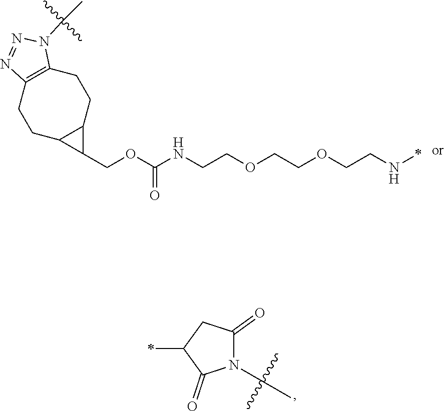 [SMILES: *NCCOCCOCCNC(=O)OCC1C2CCc3nnn(*)c3CCC21 ; *C1CC(=O)N(*)C1=O]
wherein —* is a bond to the antibody or antigen-binding fragment thereof.

8. The antibody-drug conjugate of claim 7, wherein the antibody or antigen-binding fragment thereof is joined to the conjugate linker (L) by an attachment group selected from: 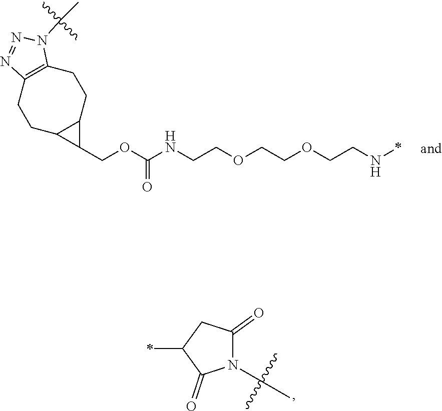 [SMILES: *NCCOCCOCCNC(=O)OCC1C2CCc3nnn(*)c3CCC21 ; *C1CC(=O)N(*)C1=O]
wherein —* is a bond to the antibody or antigen-binding fragment thereof, and wherein 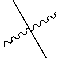
is a bond to the bridging spacer group.

9. The antibody-drug conjugate of claim 8, wherein the bridging spacer group is —CH₂CH₂—O—CH₂CH₂—CO—.

10. The antibody-drug conjugate of claim 8 or 9, wherein the bridging spacer group is joined to a cleavable group; optionally the cleavable group is -pyrophosphate-CH₂—CH₂—NH₂—.

11. The antibody-drug conjugate of any one of claims 8 to 10, wherein the cleavable group is joined to the panRAS inhibitor (D).

12. The antibody-drug conjugate of any one of claims 1 to 3, wherein the conjugate linker comprises:
an attachment group,
at least one bridging spacer group,
a peptide group, and
at least one cleavable group.

13. The antibody-drug conjugate of claim 12, wherein -(L-D) is of the formula (B): 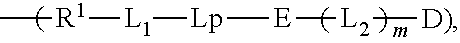
wherein:
R¹ is an attachment group;
L₁ is a bridging spacer;
Lp is a peptide group comprising 1 to 6 amino acid residues or Lp comprises a group 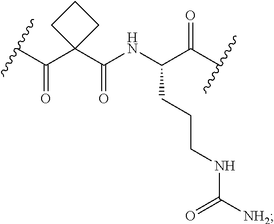 [SMILES: *C(=O)[C@H](CCCNC(N)=O)NC(=O)C1(C(*)=O)CCC1]
E is a cleavable group;
L₂ is a bridging spacer;
m is 0 or 1; and
D is a panRAS inhibitor.

14. The antibody-drug conjugate of claim 12 or 13, wherein (i) the attachment group is formed from at least one reactive group comprising a maleimide group, thiol group, cyclooctyne group, and/or an azido group, optionally wherein:
a) the maleimide group has the structure: 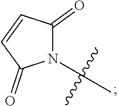 [SMILES: *N1C(=O)C=CC1=O]
b) the azido group has the structure: —N═N⁺═N⁻;
c) the cyclooctyne group has the structure: 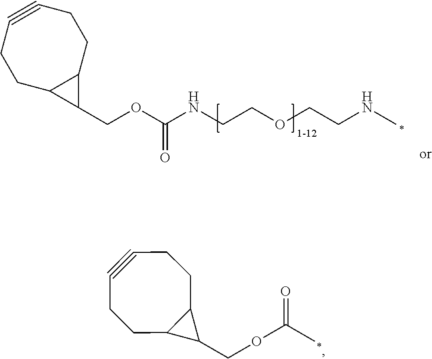
and wherein —* is a bond to the antibody or antigen-binding fragment thereof;
or
(ii) the attachment group has a formula comprising: 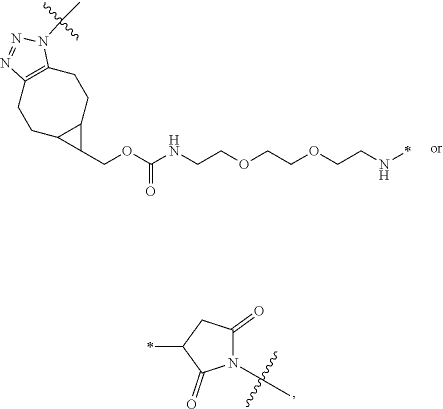 [SMILES: *NCCOCCOCCNC(=O)OCC1C2CCc3nnn(*)c3CCC21 ; *C1CC(=O)N(*)C1=O]
and
wherein —* is a bond to the antibody or antigen-binding fragment thereof.

15. The antibody-drug conjugate of any one of claims 12 to 14, wherein:
(i) at least one bridging spacer comprises a PEG group, optionally the PEG group is selected from, PEG1, PEG2, PEG3, PEG4, PEG5, PEG6, PEG7, PEG8, PEG9, PEG10, PEG11, PEG12, PEG13, PEG14, and PEG15; or
(ii) at least one bridging spacer is selected from *—C(O)—CH2-CH2-PEG1-**, *—C(O)—CH₂-PEG3-**, *—C(O)—CH₂—CH₂—PEG12**, *—NH—CH₂—CH₂—PEG1-**, a polyhydroxyalkyl group, *—C(O)—N(CH3)-CH2-CH2-N(CH₃)—C(O)—**, and *—C(O)—CH₂—CH₂—PEG12-NH—C(O)CH₂—CH₂—**, wherein ** indicates the point of direct or indirect attachment of the at least one bridging spacer to the attachment group and * indicates the point of direct or indirect attachment of the at least one bridging spacer to the peptide group.

16. The antibody-drug conjugate of any one of claims 12 to 15, wherein L₁ is selected from *—C(O)—CH₂—CH₂—PEG1-**, *—C(O)—CH₂—PEG3-**, *—C(O)—CH₂—CH₂—PEG12**, *—NH—CH₂—CH₂-PEG1-**, and a polyhydroxyalkyl group, wherein ** indicates the point of direct or indirect attachment of L₁ to R¹ and * indicates the point of direct or indirect attachment of L₁ to Lp.

17. The antibody-drug conjugate of any one of claims 12 to 16, wherein m is 1 and L₂ is —C(O)—N(CH₃)—CH₂—CH₂—N(CH₃)—C(O)—.

18. The antibody-drug conjugate of any one of claims 12 to 17, wherein
(i) the peptide group comprises 1 to 6, 1 to 4, 1 to 3 or 1 to 2 amino acid residues, optionally the amino acid residues are selected from L-glycine (Gly), L-valine (Val), L-citrulline (Cit), L-cysteic acid (sulfo-Ala), L-lysine (Lys), L-isoleucine (lie), L-phenylalanine (Phe), L-methionine (Met), L-asparagine (Asn), L-proline (Pro), L-alanine (Ala), L-leucine (Leu), L-tryptophan (Trp), and L-tyrosine (Tyr);
(ii) the peptide group comprises Val-Cit, Val-Ala, Val-Lys, and/or sulfo-Ala-Val-Ala;
(iii) the peptide group is selected from:

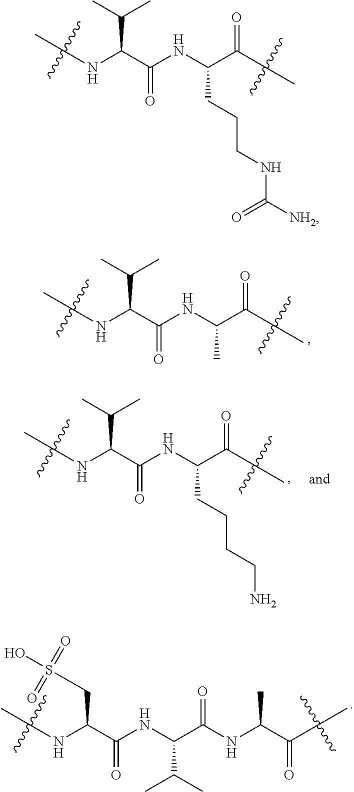

19. The antibody-drug conjugate of any one of claims 12 to 18, wherein (i) the cleavable group comprises a pyrophosphate and/or a self-immolative group; (ii) the cleavable group comprises a self-immolative group; or (iii) the cleavable group comprises a self-immolative group comprising para-aminobenzyl-carbamate, para-aminobenzyl-ammonium, para-amino-(sulfo)benzyl-ammonium, para-amino-(sulfo)benzyl-carbamate, para-amino-(alkoxy-PEG-alkyl)benzyl-carbamate, para-amino-(polyhydroxycarboxytetrahydropyranyl)alkyl-benzyl-carbamate, or para-amino-(polyhydroxycarboxytetrahydropyranyl)alkyl-benzyl-ammonium.

20. The antibody-drug conjugate of any one of claims 13 to 19, wherein m is 0 or 1 or m is 1 and the bridging spacer comprises

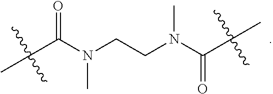 [SMILES: *C(=O)N(C)CCN(C)C(*)=O]

21. The antibody-drug conjugate of any one of claims 13-20, wherein -(L-D) is formed from a compound selected from:

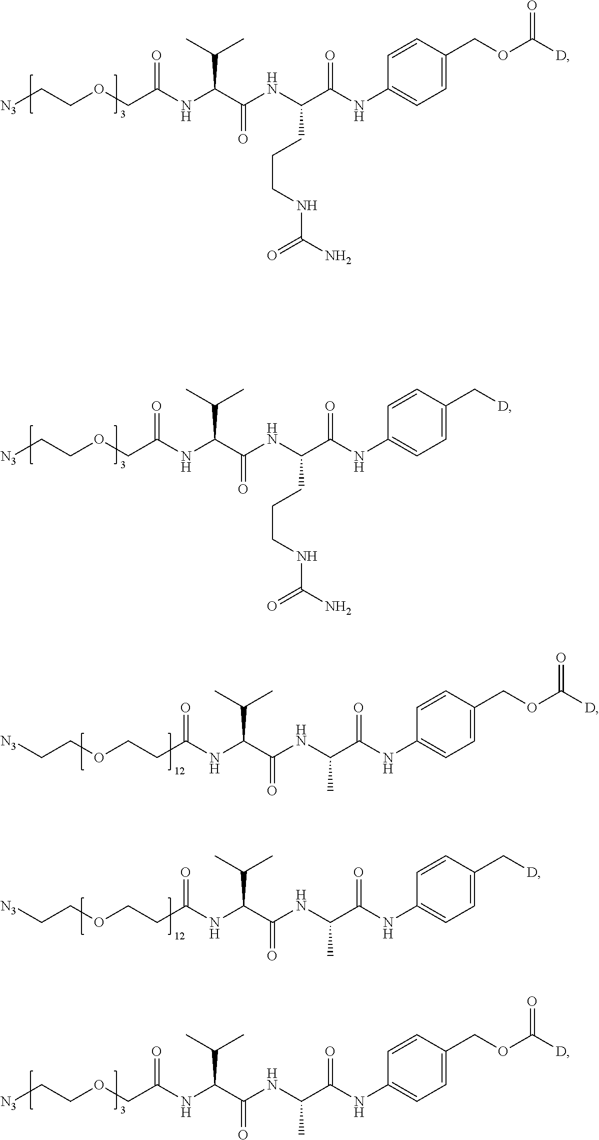

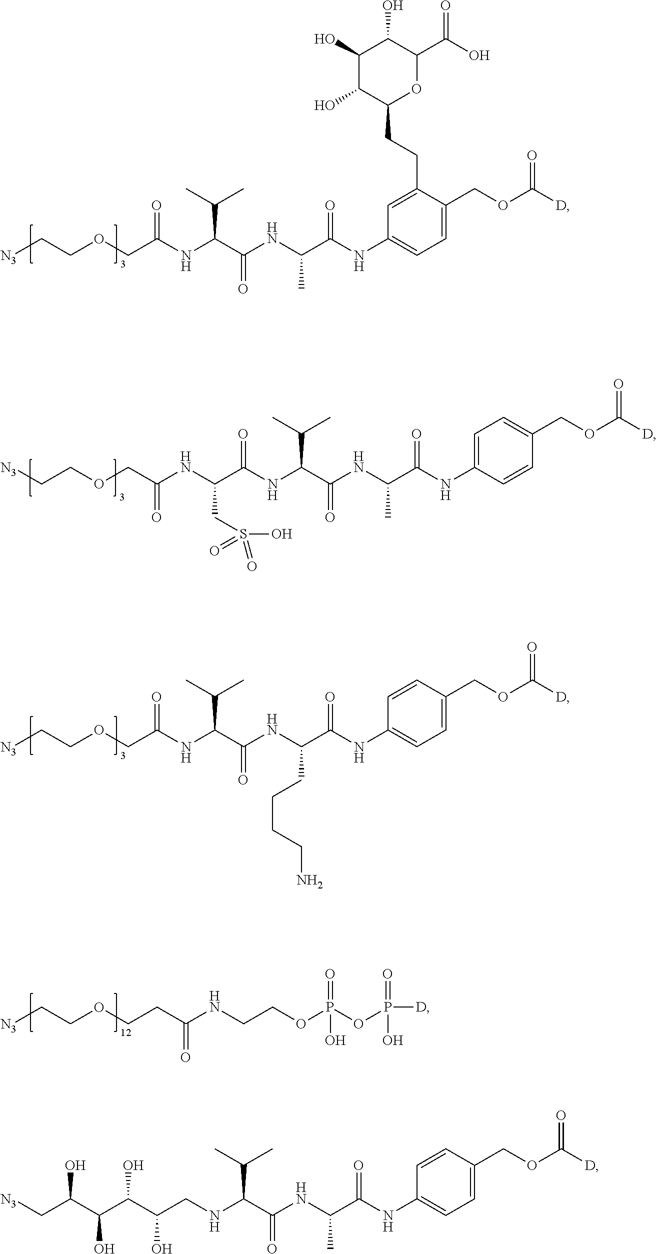

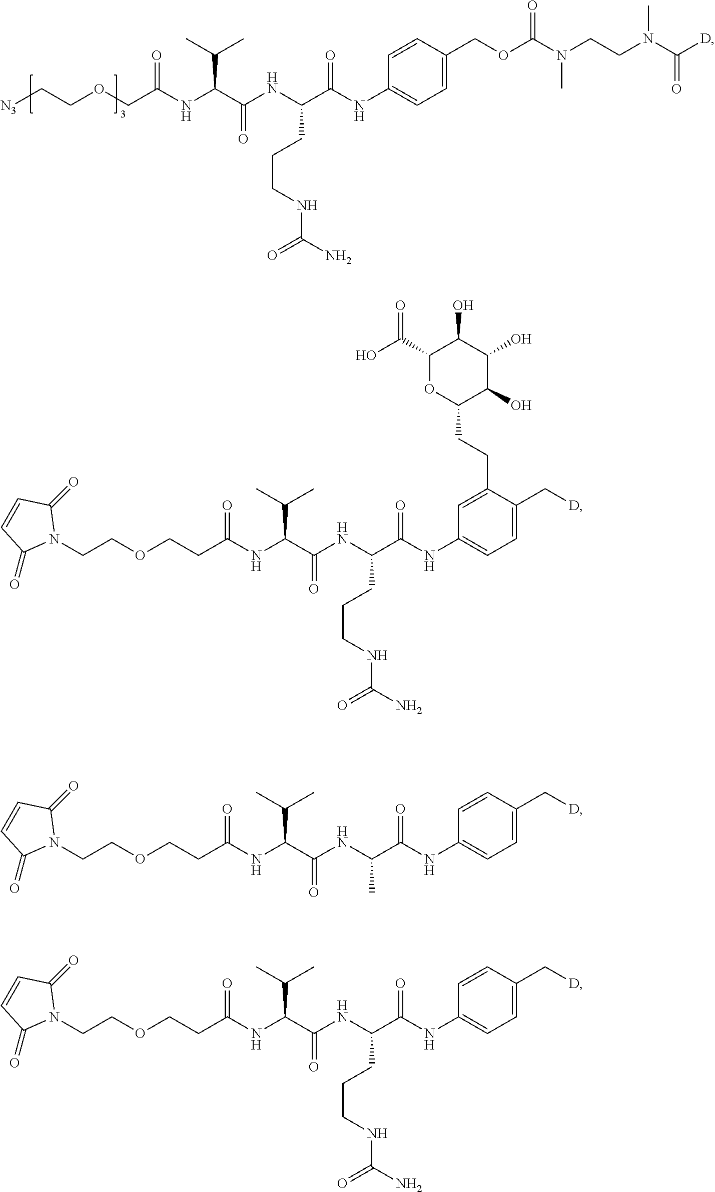

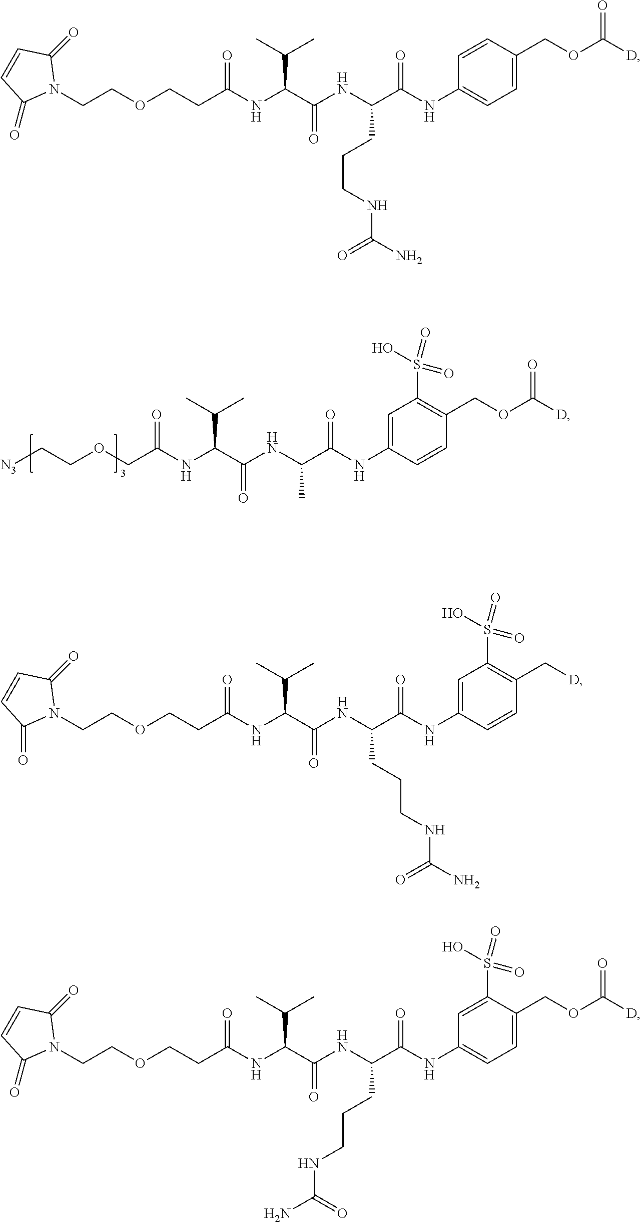

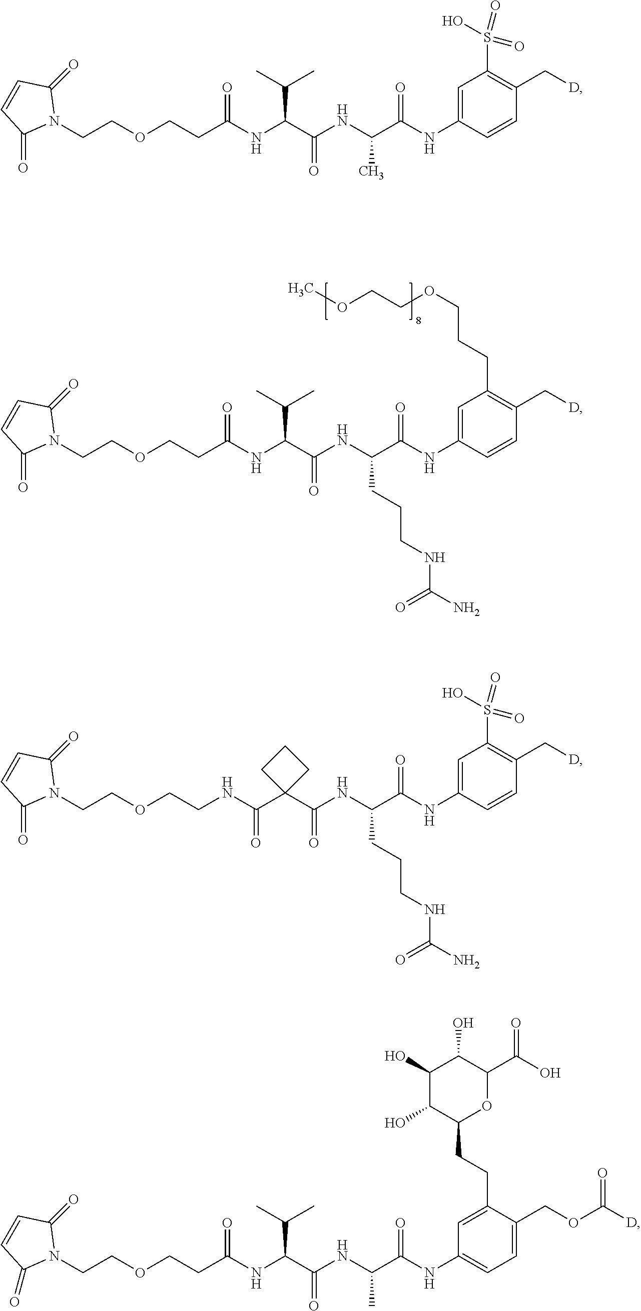

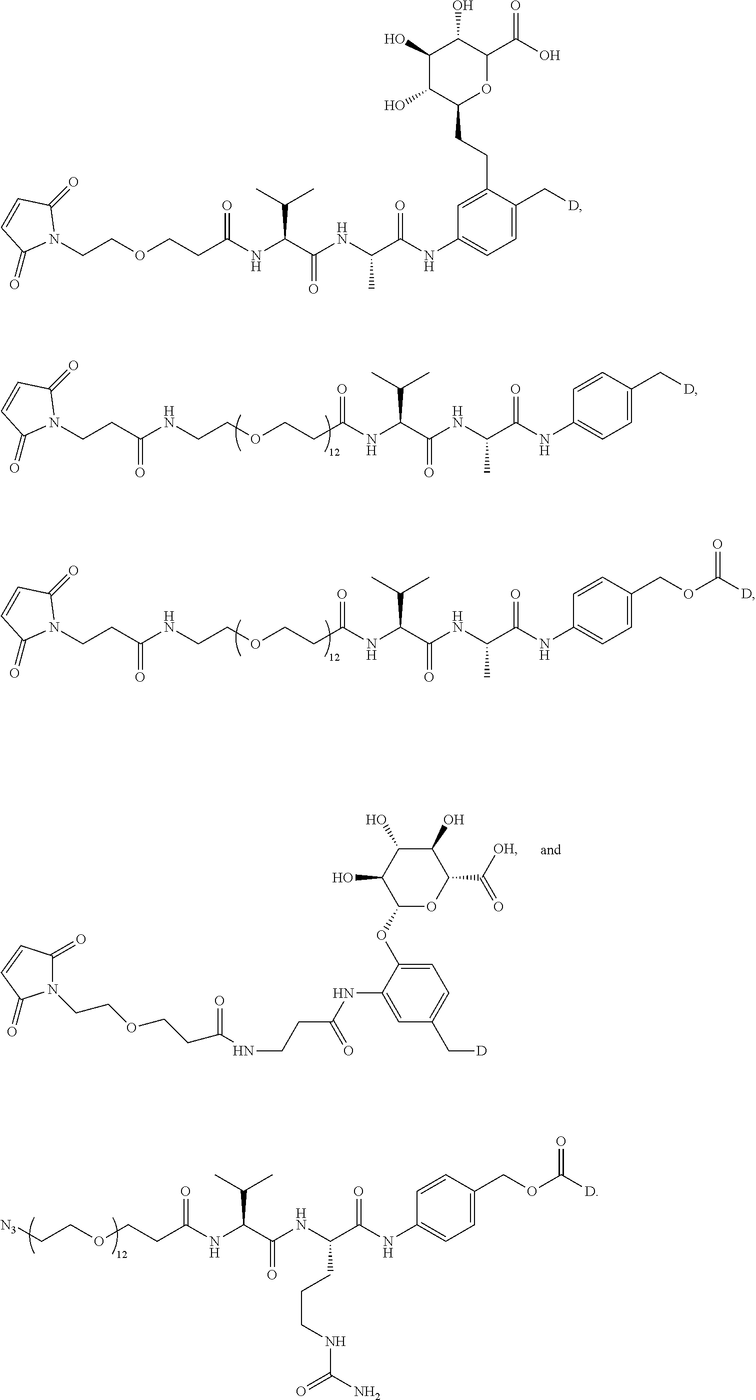

22. The antibody-drug conjugate of any one of claims 13-21, wherein -(L-D) comprises a formula selected from: 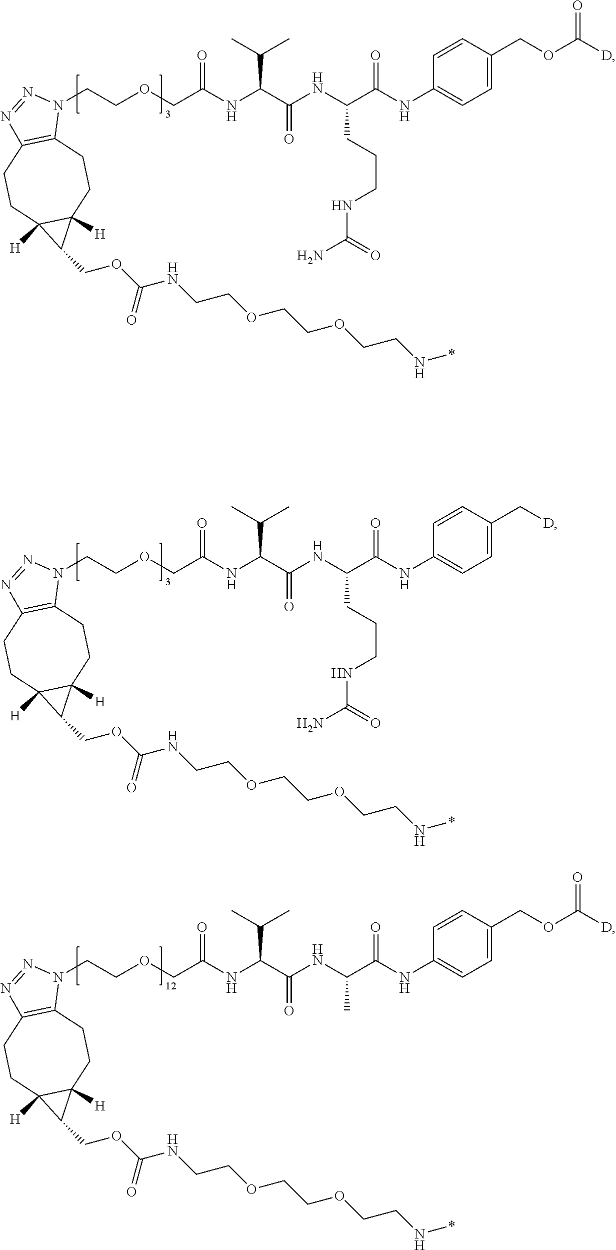 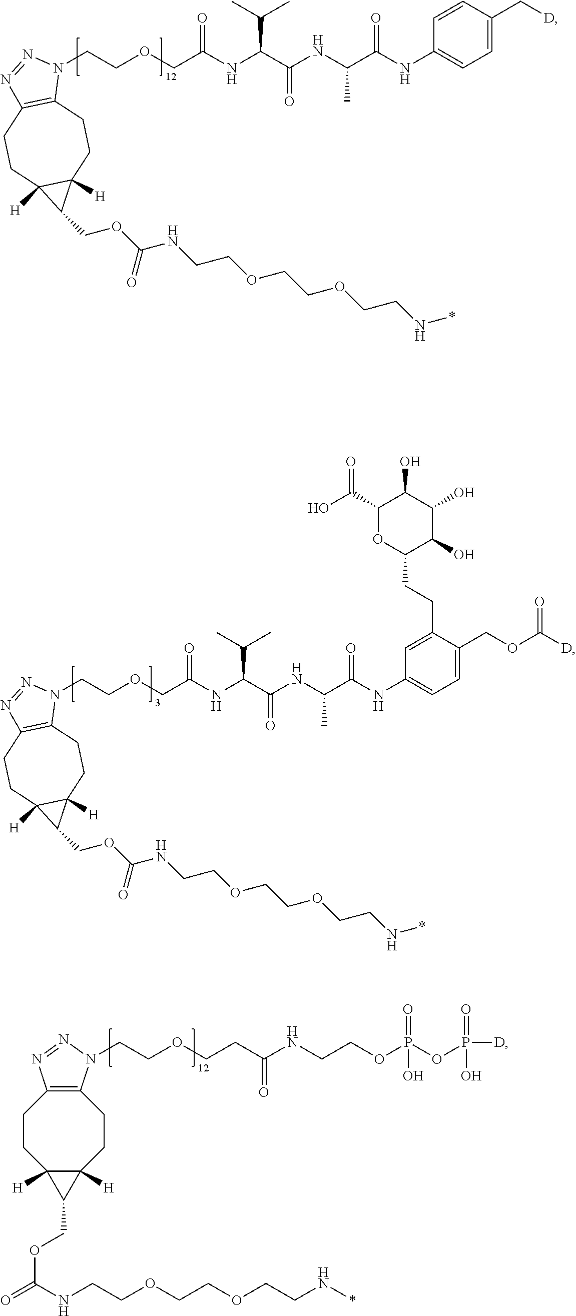 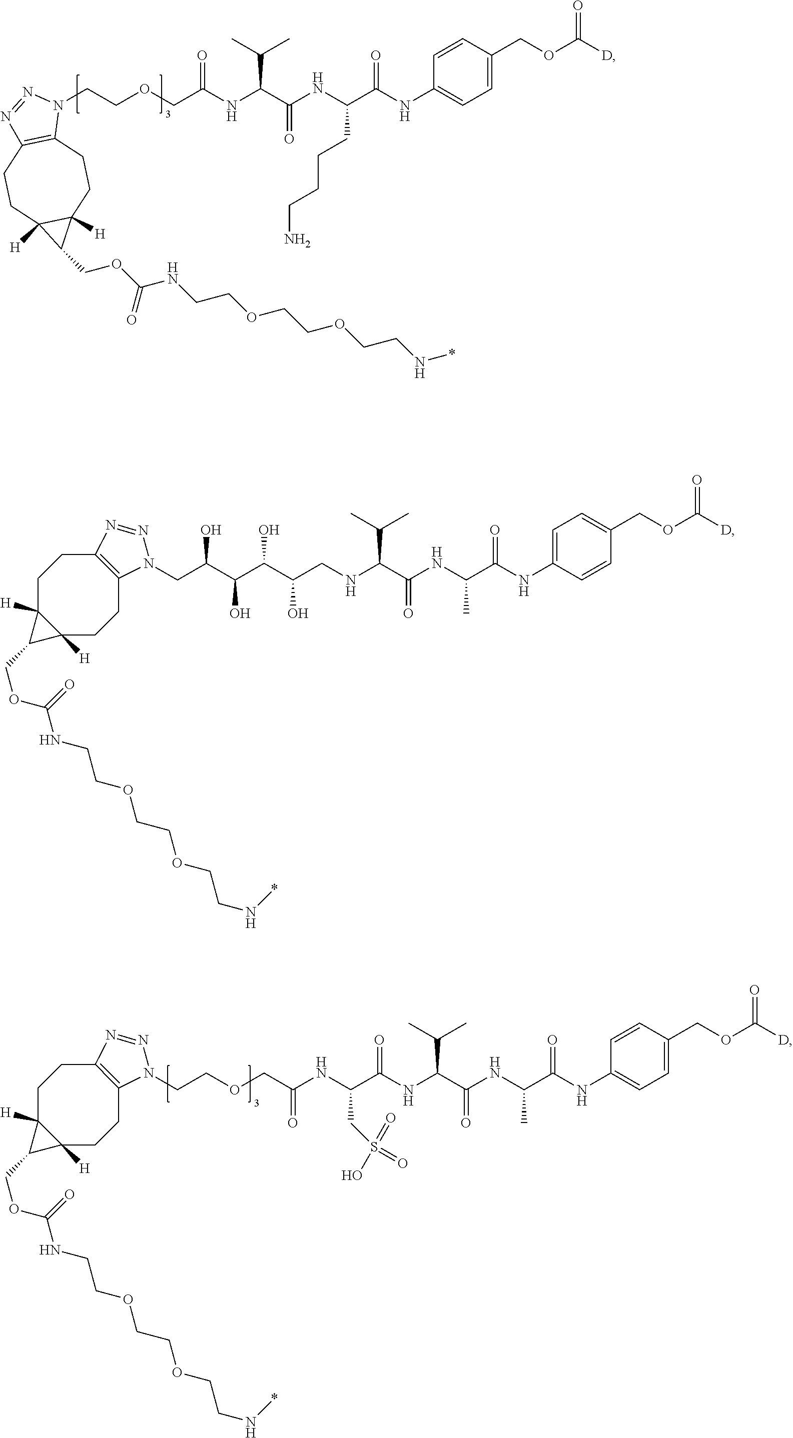 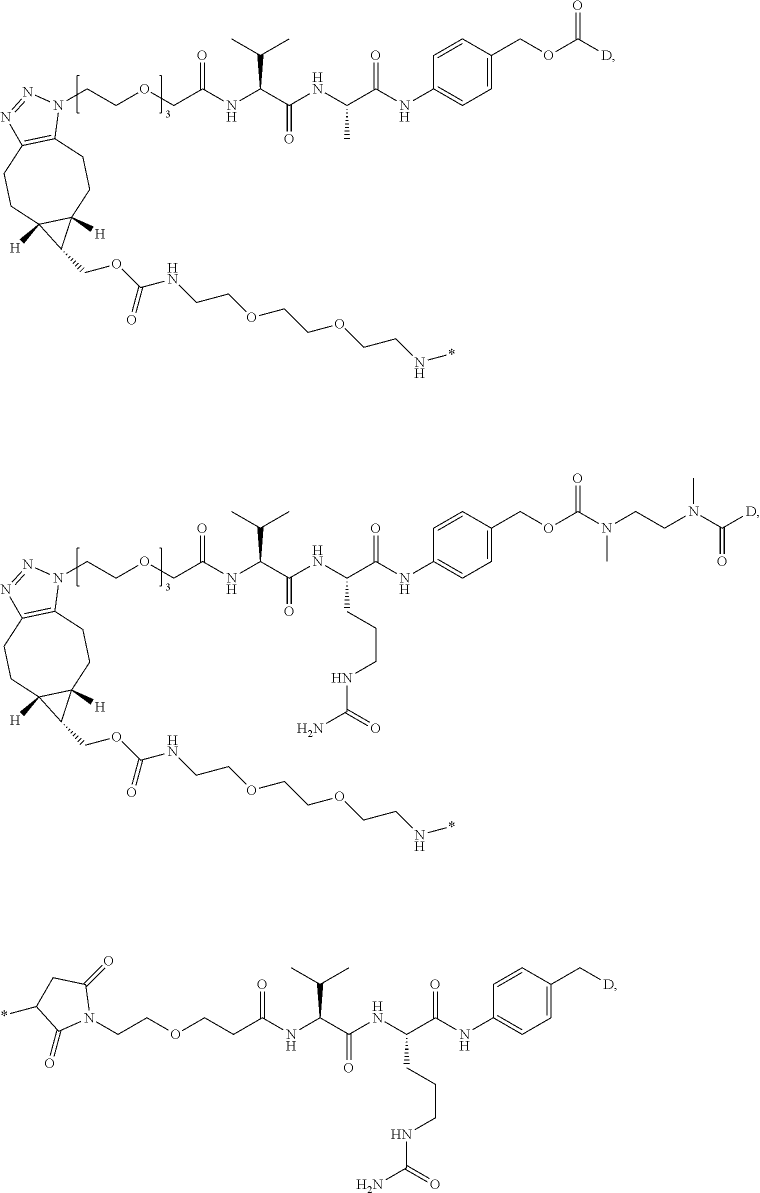 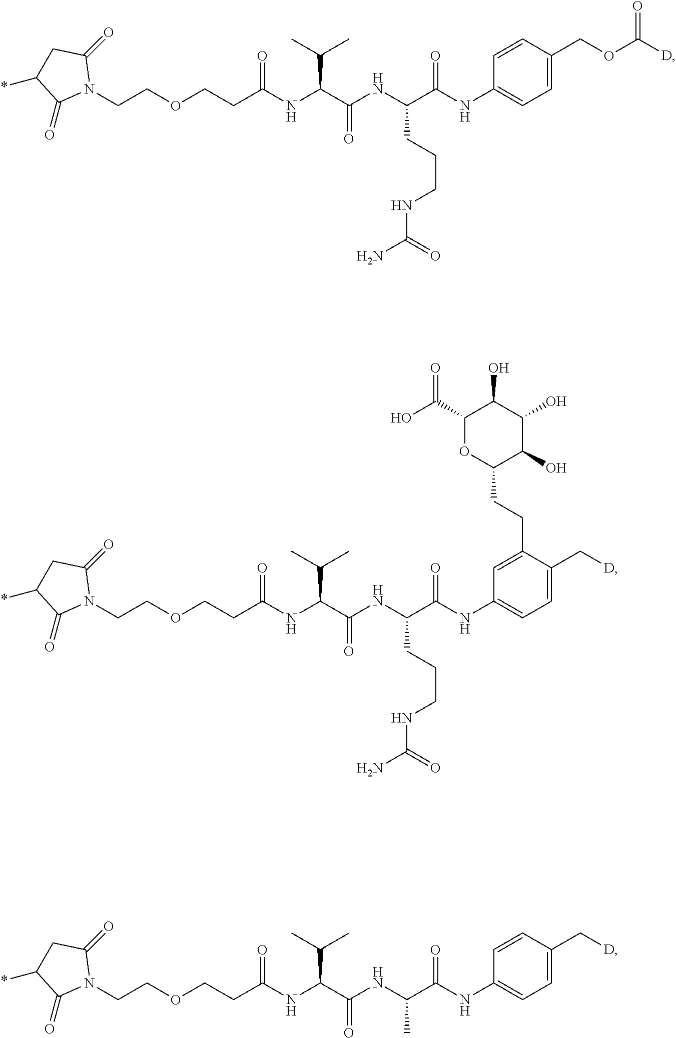 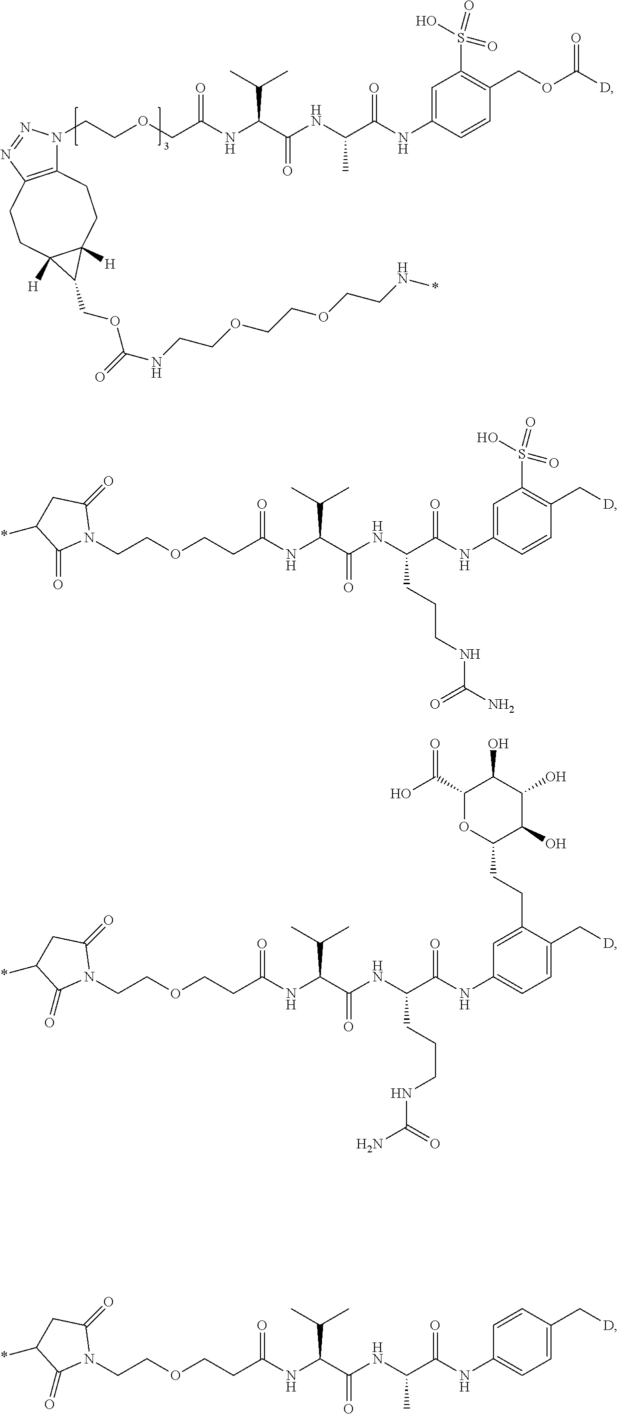 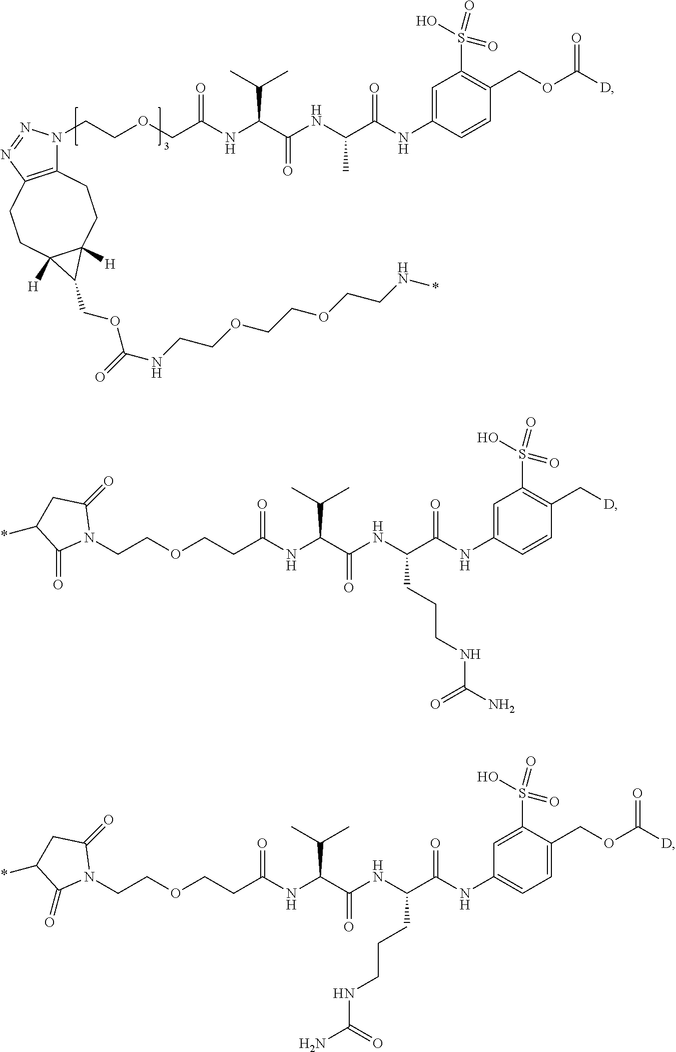 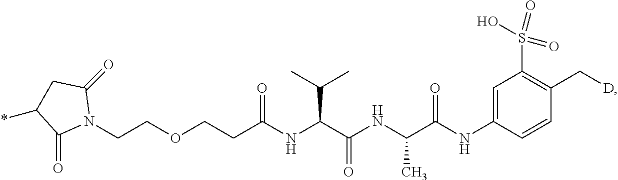
and
wherein —* is a bond to the antibody or antigen-binding fragment thereof.

23. The antibody-drug conjugate of claim 1 or 2, wherein -(L-D) is of the formula (C): 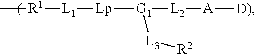
wherein:
R¹ is an attachment group;
L₁ is a bridging spacer;
Lp is a peptide group comprising 1 to 6 amino acids;
D is a panRAS inhibitor;
G₁-L₂-A is a self-immolative spacer;
L₂ is a bond, a methylene, a neopentylene or a C₂-C₃ alkenylene;
A is a bond, —OC(═O)—*, 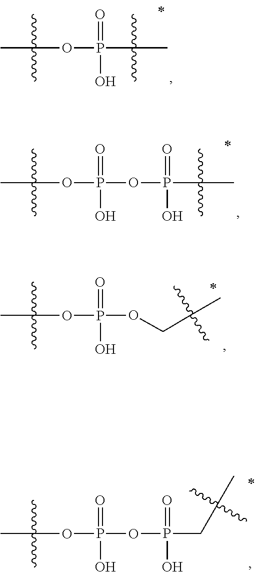 [SMILES: *OP(*)(=O)O ; *OP(=O)(O)OP(*)(=O)O ; *COP(=O)(O)O* ; *CP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D;
L₃ is a spacer moiety; and
R² is a hydrophilic moiety.

24. The antibody-drug conjugate of claim 23, or pharmaceutically acceptable salt thereof, wherein -(L-D) is of Formula (D): 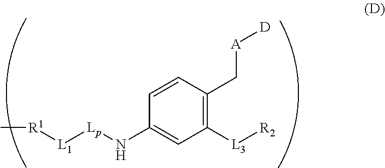
wherein:
R¹ is an attachment group;
L₁ is a bridging spacer;
Lp is a peptide group comprising 1 to 6 amino acids;
A is a bond, —OC(═O)—*, 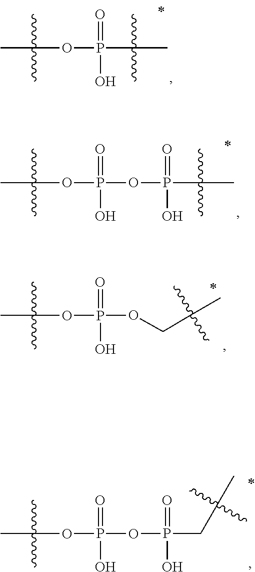 [SMILES: *OP(*)(=O)O ; *OP(=O)(O)OP(*)(=O)O ; *COP(=O)(O)O* ; *CP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D;
L₃ is a spacer moiety; and
R² is a hydrophilic moiety.

25. The antibody-drug conjugate of claim 23 or 24, wherein:
(1) L₁ comprises: 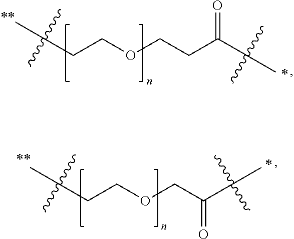
or
*—CH(OH)CH(OH)CH(OH)CH(OH)—**,
wherein each n is an integer from 1 to 12, wherein the * of L₁ indicates the point of direct or indirect attachment to Lp, and the ** of L₁ indicates the point of direct or indirect attachment to R¹;
(2) L₁ is 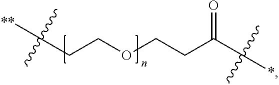
and n is an integer from 1 to 12 or n is 1 or n is 12, wherein the * of L₁ indicates the point of direct or indirect attachment to Lp, and the ** of L₁ indicates the point of direct or indirect attachment to R¹;
(3) L₁ is 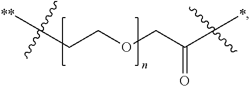
and n is an integer from 1 to 12, wherein the * of L₁ indicates the point of direct or indirect attachment to Lp, and the ** of L₁ indicates the point of direct or indirect attachment to R¹;
(4) L₁ comprises 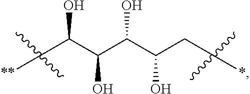
wherein the * of L₁ indicates the point of direct or indirect attachment to Lp, and the ** of L₁ indicates the point of direct or indirect attachment to R¹;
(5) L₁ is a bridging spacer comprising:
—C(═O)(CH₂)ₘO(CH₂)ₘ—**; *—C(═O)((CH₂)ₘO)ₜ(CH₂)ₙ—**; *—C(═O)(CH₂)ₘ—**; *—C(═O)N H((CH₂)ₘO)ₜ(CH₂)ₙ—**; *—C(═O)O(CH₂)ₘSSC(R³)₂(CH₂)ₘC(═O)NR³(CH₂)ₘNR³C(═O)(CH₂)ₘ—**; *—C(═O)O(CH₂)ₘC(═O)NH(CH₂)ₘ—**; *—C(═O)(CH₂)ₘNH(CH₂)ₘ—**; *—C(═O)(CH₂)ₘNH(CH₂)ₙC(═O)—**; *—C(═O)(CH₂)ₘX₁(CH₂)ₘ—**; *—C(═O)((CH₂)ₘO)ₜ(CH₂)ₙX₁(CH₂)ₙ—**; *—C(═O)(CH₂)ₘNHC(═O)(CH₂)ₙ—**; *—C(═O)((CH₂)ₘO)ₜ(CH₂)ₙNHC(═O)(CH₂)ₙ—**; *—C(═O)(CH₂)ₘNHC(═O)(CH₂)ₙX₁(CH₂)ₙ—**; *—C(═O)((CH₂)ₘO)ₜ(CH₂)ₙNHC(═O)(CH₂)ₙX₁(CH₂)ₙ—**; *—C(═O)((CH₂)ₘO)ₜ(CH₂)ₙC(═O)NH(CH₂)ₘ—**; *—C(═O)(CH₂)ₘC(R³)₂—** or *—C(═O)(CH₂)ₘC(═O)NH(CH₂)ₘ—**, wherein the * of L₁ indicates the point of direct or indirect attachment to Lp, and the ** of L₁ indicates the point of direct or indirect attachment to R¹;
X₁ is 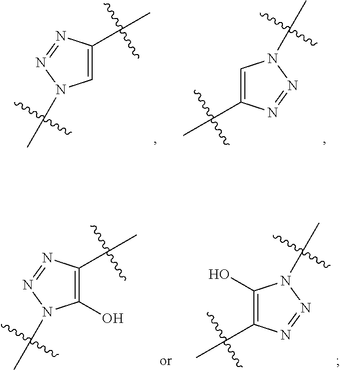 [SMILES: *c1cn(*)nn1 ; *c1cn(*)nn1 ; *c1nnn(*)c1O ; *c1nnn(*)c1O]
and
each m is independently selected from 1, 2, 3, 4, 5, 6, 7, 8, 9 and 10;
each n is independently selected from 1, 2, 3, 4, 5, 6, 7, 8, 9 and 10; and
each t is independently selected from 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30:
and each R³ is independently selected from H and C₁-C₆alkyl.

26. The antibody-drug conjugate of any one of claims 23 to 25, wherein R² is a hydrophilic moiety comprising polyethylene glycol, polyalkylene glycol, a polyol, a polysarcosine, a sugar, an oligosaccharide, a polypeptide, C₂-C₅ alkyl substituted with 1 to 3 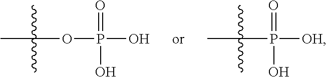 [SMILES: *OP(=O)(O)O ; *P(=O)(O)O]
or C₂-C₆alkyl substituted with 1 to 2 substituents independently selected from —OC(═O)NHS(O)₂NHCH₂CH₂OCH₃, —NHC(═O)C₁₋₄alkylene-P(O)(OCH₂CH₃)₂ and —COOH groups.

27. The antibody-drug conjugate of any one of claims 23 to 26, wherein R² is 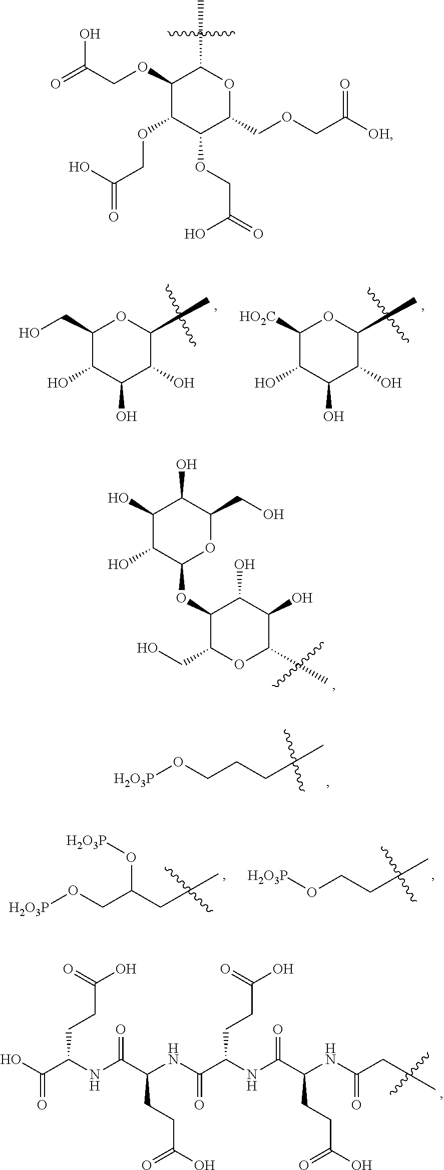 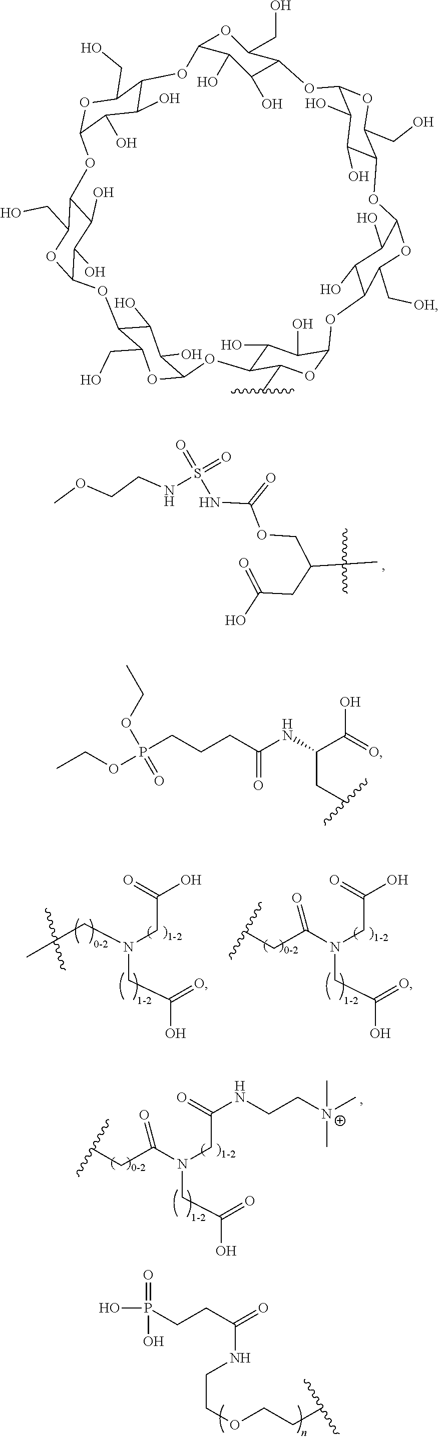
wherein n is an integer between 1 and 6,

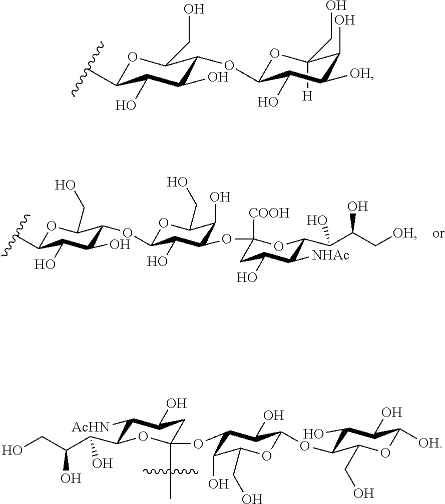

28. The antibody-drug conjugate of claim 23 or 24, wherein the hydrophilic moiety comprises:
(i) a polysarcosine with the following moiety: 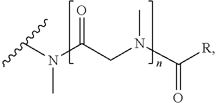
wherein n is an integer between 3 and 25; and R is H, —CH₃ or —CH₂CH₂C(═O)OH; or
(ii) a polyethylene glycol of formula: 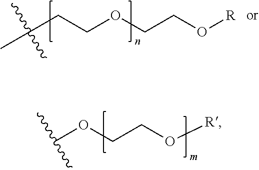
wherein R is H, —CH₃, CH₂CH₂NHC(═O)ORₐ, —CH₂CH₂NHC(═O)Rₐ, or —CH₂CH₂C(═O)ORₐ, R′ is OH, —OCH₃, —CH₂CH₂NHC(═O)ORₐ, —CH₂CH₂NHC(═O)Rₐ, or —OCH₂CH₂C(═O)ORₐ, in which Rₐ is H or C₁0.4 alkyl optionally substituted with either OH or C₁₋₄ alkoxyl, and each of m and n is independently an integer between 2 and 25.

29. The antibody-drug conjugate of any one of claims 23 to 27, wherein the hydrophilic moiety comprises

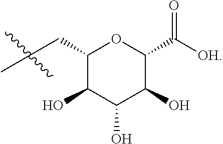 [SMILES: *C[C@@H]1O[C@H](C(=O)O)[C@@H](O)[C@H](O)[C@H]1O]

30. The antibody-drug conjugate of any one of claims 23 to 29, wherein:
(i) L₃ is a spacer moiety having the structure 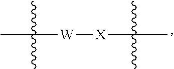
wherein:
W is —CH₂—, —CH₂O—, —CH₂N(R^{b})C(═O)O—, —NHC(═O)C(R^{b})₂NHC(═O)O—, —NHC(═O)C(R^{b})₂NH—, —NHC(═O)C(R^{b})₂NHC(═O)—, —CH₂N(X—R²)C(═O)O—, —C(═O)N(X—R²)—, —CH₂N(X—R²)C(═O)—, —C(═O)NR^{b}—, —C(═O)NH—, —CH₂N Rb C(═O)—, —CH₂NR^{b} C(═O)NH—, —CH₂NR^{b}C(═O)NR^{b}—, —NHC(═O)—, —NHC(═O)O—, —NHC(═O)NH—, —OC(═O)NH—, —S(O)₂NH—, —NHS(O)₂—, —C(═O)—, —C(═O)O— or —NH—, wherein each R^{b} is independently selected from H, C₁-C₆alkyl, and C₃-C₈ cycloalkyl; and
X is a bond, triazolyl, or —CH₂-triazolyl-,
wherein X is connected to R²; or
(ii) L₃ is a spacer moiety having the structure 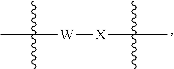
wherein:
W is —CH₂—, —CH₂O—, —CH₂N(R^{b})C(═O)O—, —NHC(═O)C(R^{b})₂NHC(═O)O—, —NHC(═O)C(R^{b})₂NH—, —NHC(═O)C(R^{b})₂NHC(═O)—, —CH₂N(X—R²)C(═O)O—, —C(═O)N(X—R²)—, —CH₂N(X—R²)C(═O)—, —C(═O)NR^{b}—, —C(═O)NH—, —CH₂NR^{b}C(═O)—, —CH₂NR^{b}C(═O)NH—, —CH₂NR^{b}C(═O)NR^{b}—, —NHC(═O)—, —NHC(═O)O—, —NHC(═O)NH—, —OC(═O)NH—, —S(O)₂NH—, —NHS(O)₂—, —C(═O)—, —C(═O)O— or —NH—, wherein each R^{b} is independently selected from H, C₁-C₆alkyl, and C₃-C₈cycloalkyl; and
X is —CH₂-triazolyl-C₁₋₄ alkylene-OC(O)NHS(O)₂NH—, —C₄63 cycloalkylene-OC(O)NHS(O)₂NH—, —(CH₂CH₂O)ₙ—C(O)NHS(O)₂NH—, —(CH₂CH₂O)ₙ—C(O)NHS(O)₂NH—(CH₂CH₂O)ₙ—, —CH₂-triazolyl-C₁₋₄ alkylene-OC(O)NHS(O)₂NH—(CH₂CH₂O)ₙ—, —C₄₋₆cycloalkylene-OC(O)NHS(O)₂NH—(CH₂CH₂O)ₙ—,wherein each n independently is 1, 2, or 3,
wherein X is connected to R².

31. The antibody-drug conjugate of any one of claims 3 to 30, wherein the attachment group is formed by a reaction comprising at least one reactive group.

32. The antibody-drug conjugate of any one of claims 3 to 31, wherein the attachment group is formed by reacting:
a first reactive group that is attached to the conjugate linker, and
a second reactive group that is attached to the antibody or antigen-binding fragment thereof or is an amino acid residue of the antibody or antigen-binding fragment thereof, wherein optionally,
(i) at least one of the reactive groups comprises:
a thiol,
a maleimide,
a haloacetamide,
an azide,
an alkyne,
a cyclcooctene,
a triaryl phosphine,
an oxanobornadiene,
a cyclooctyne,
a diaryl tetrazine,
a monoaryl tetrazine,
a norbornene,
an aldehyde,
a hydroxylamine,
a hydrazine,
NH₂—NH—C(═O)—,
a ketone,
a vinyl sulfone,
an aziridine,
an amino acid residue, 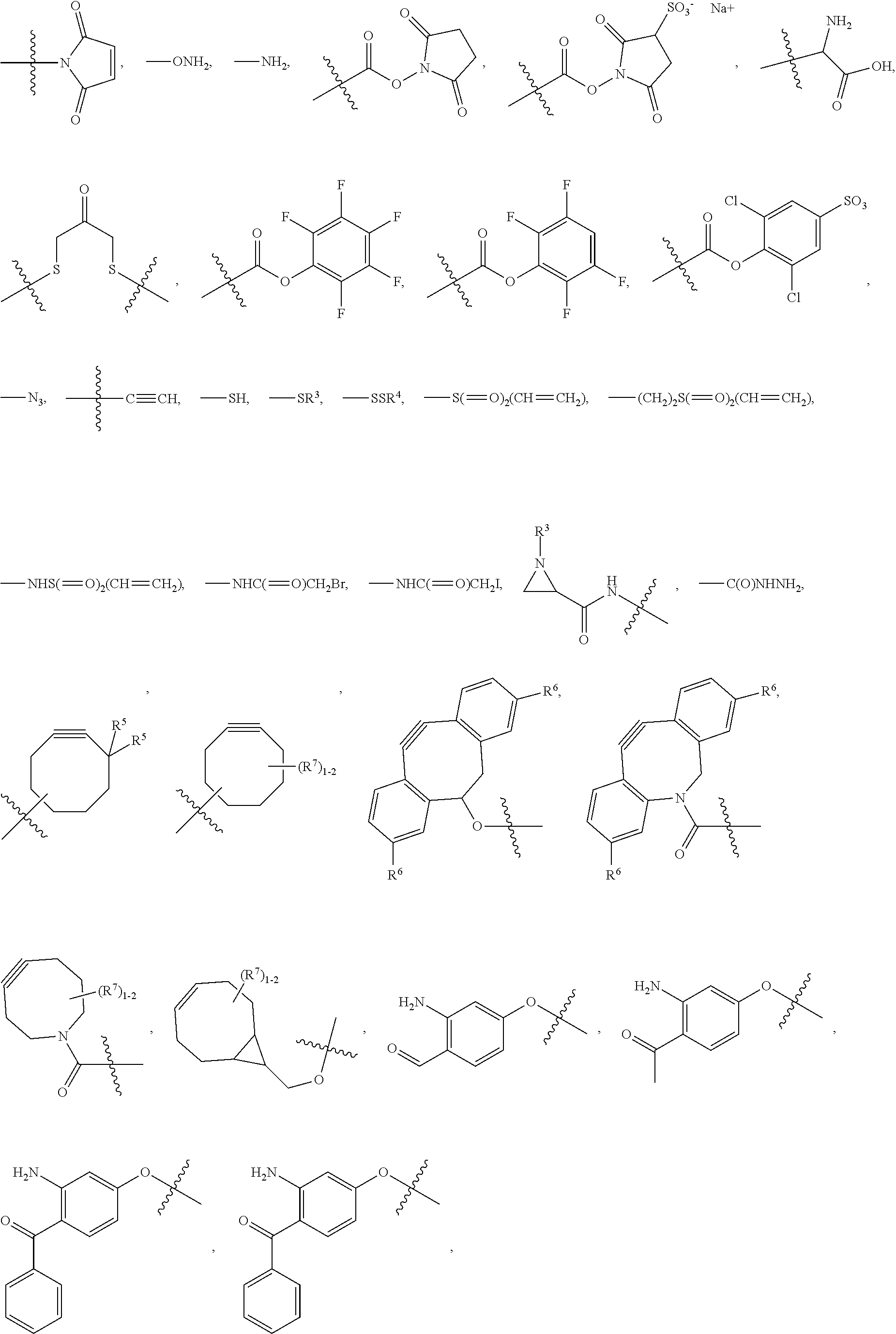 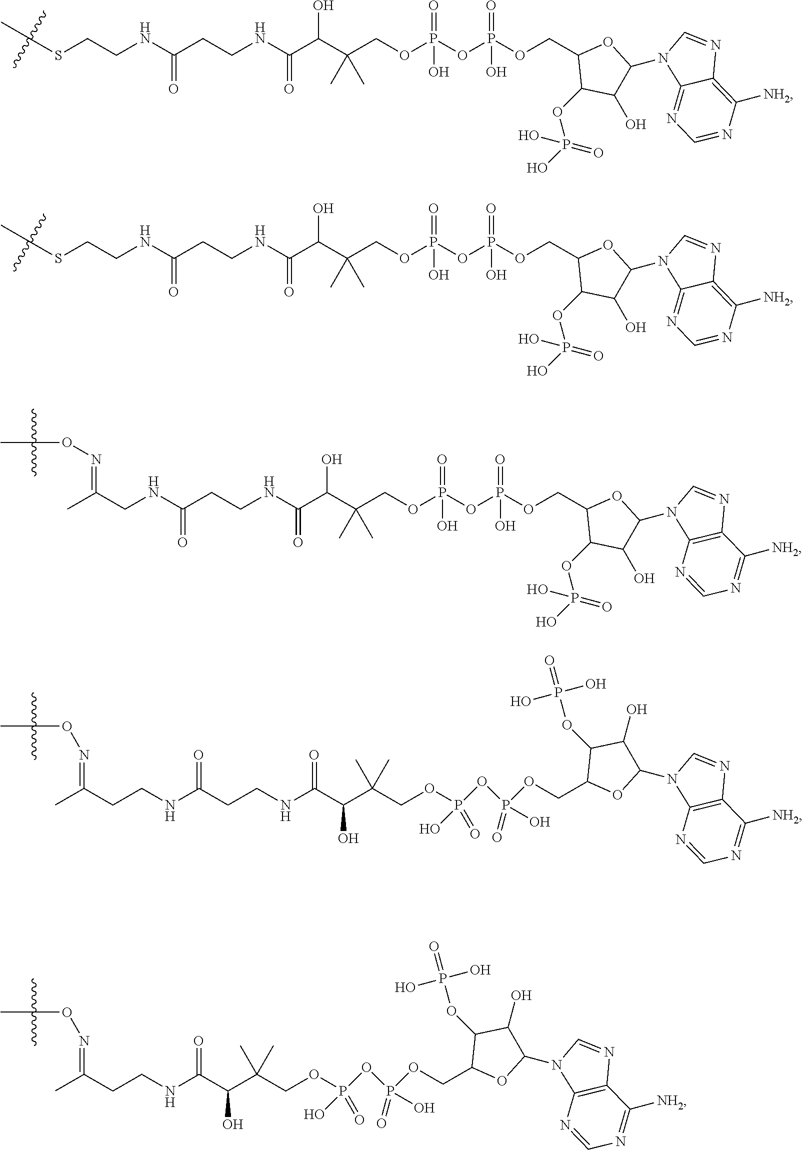 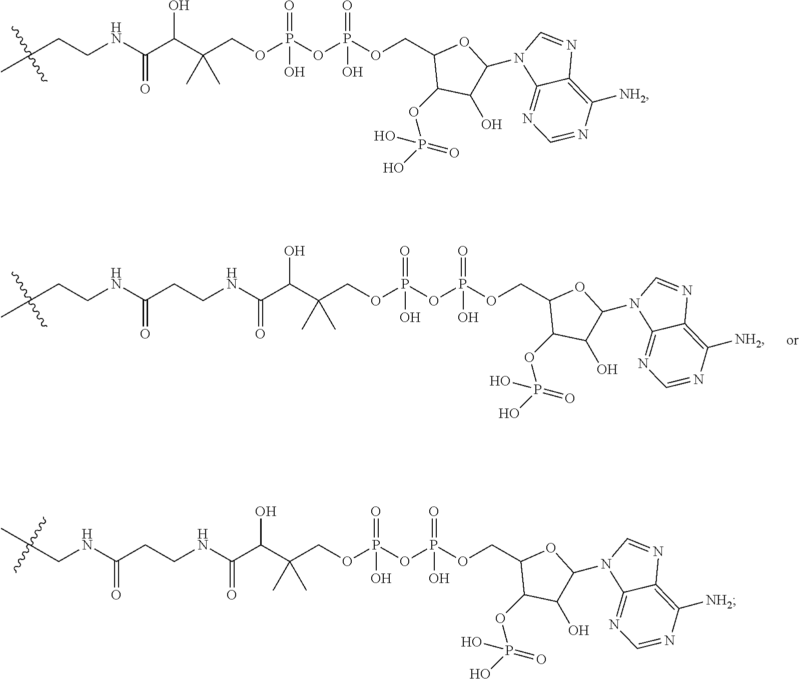
wherein:
each R³ is independently selected from H and C₁-C₆alkyl;
each R⁴ is 2-pyridyl or 4-pyridyl;
each R⁵ is independently selected from H, C₁-C₆alkyl, F, Cl, and —OH;
each R⁶ is independently selected from H, C₁-C₆alkyl, F, Cl, —NH₂, —OCH₃, —OCH₂CH₃, —N(CH₃)₂, —ON, —NO₂ and —OH;
each R⁷ is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH; and/or
(ii) the first reactive group and second reactive group comprise:
a thiol and a maleimide,
a thiol and a haloacetamide,
a thiol and a vinyl sulfone,
a thiol and an aziridine,
an azide and an alkyne,
an azide and a cyclooctyne,
an azide and a cyclooctene,
an azide and a triaryl phosphine,
an azide and an oxanobornadiene,
a diaryl tetrazine and a cyclooctene,
a monoaryl tetrazine and a nonbornene,
an aldehyde and a hydroxylamine,
an aldehyde and a hydrazine,
an aldehyde and NH₂—NH—C(═O)—,
a ketone and a hydroxylamine,
a ketone and a hydrazine,
a ketone and NH₂—NH—C(═O)—,
a hydroxylamine and 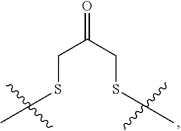 [SMILES: *SCC(=O)CS*]
an amine and 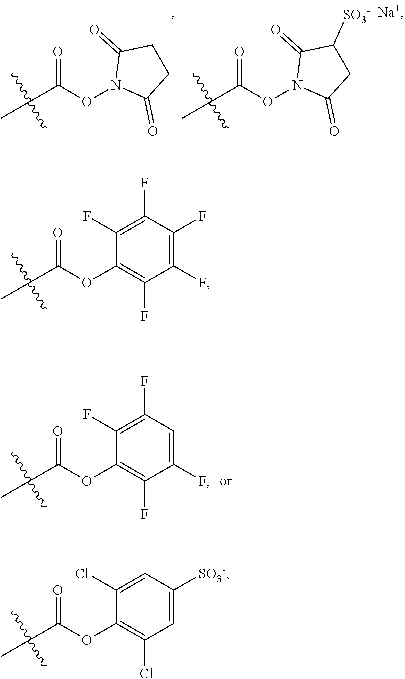
or
a CoA or CoA analogue and a serine residue.

33. The antibody-drug conjugate any one of claims 3 to 32, where the attachment group comprises a group selected from: 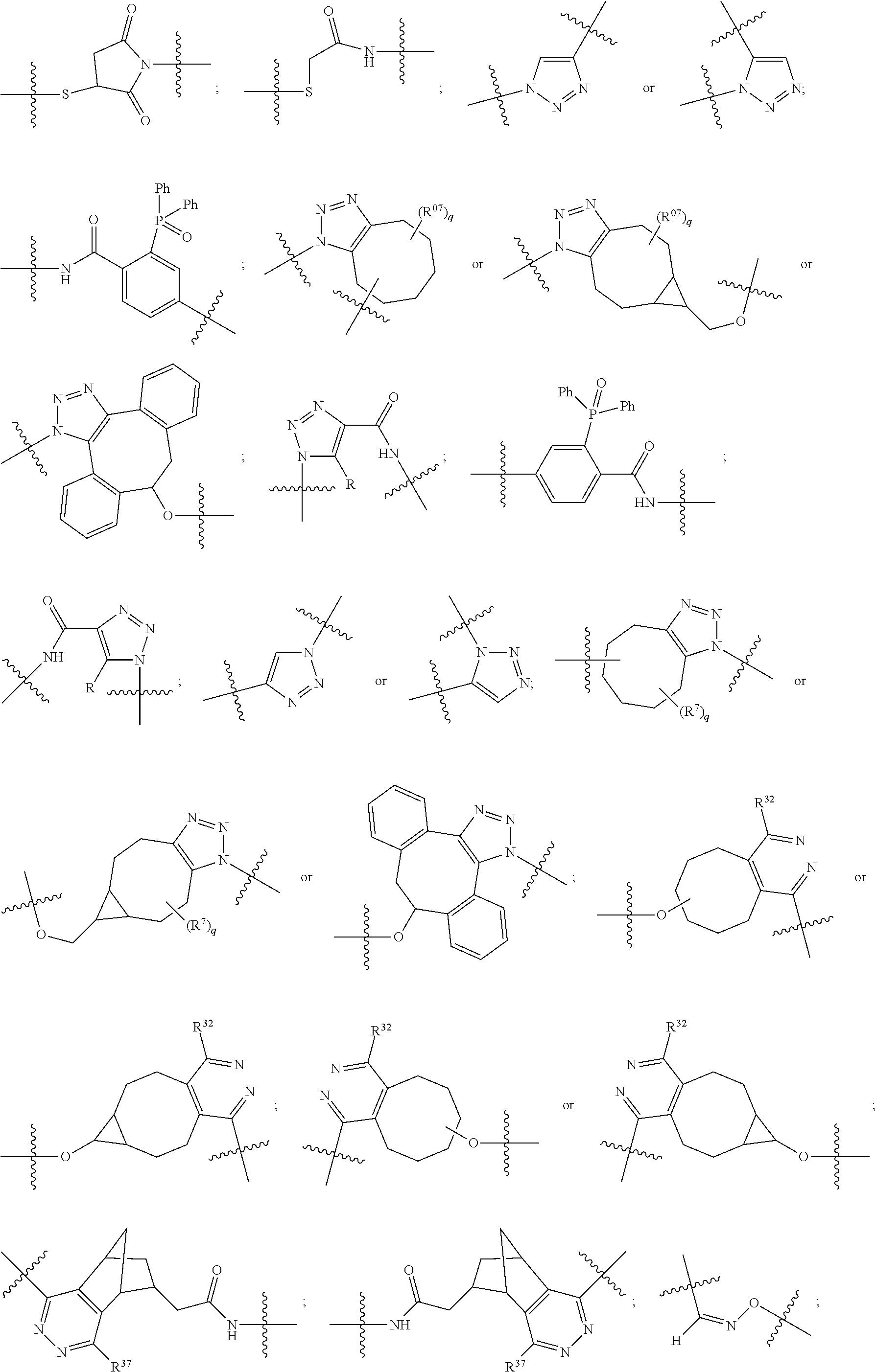 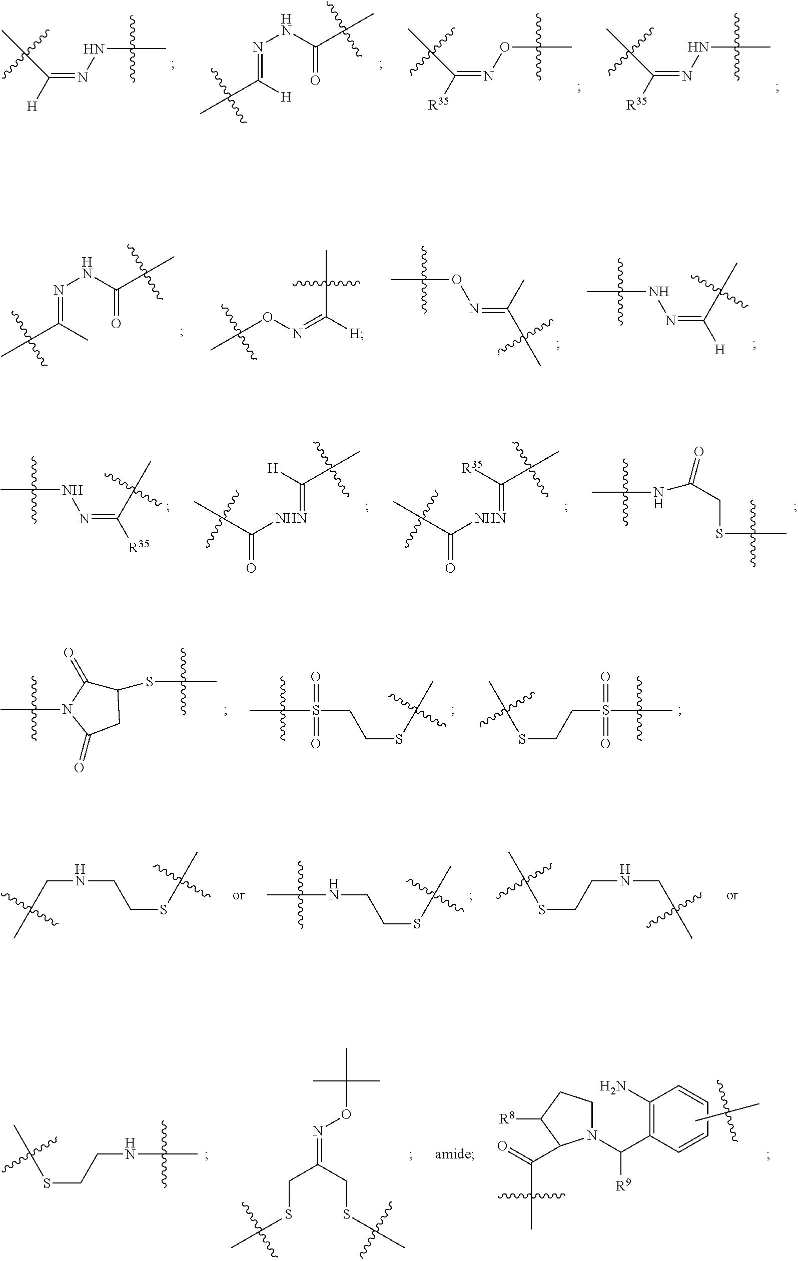 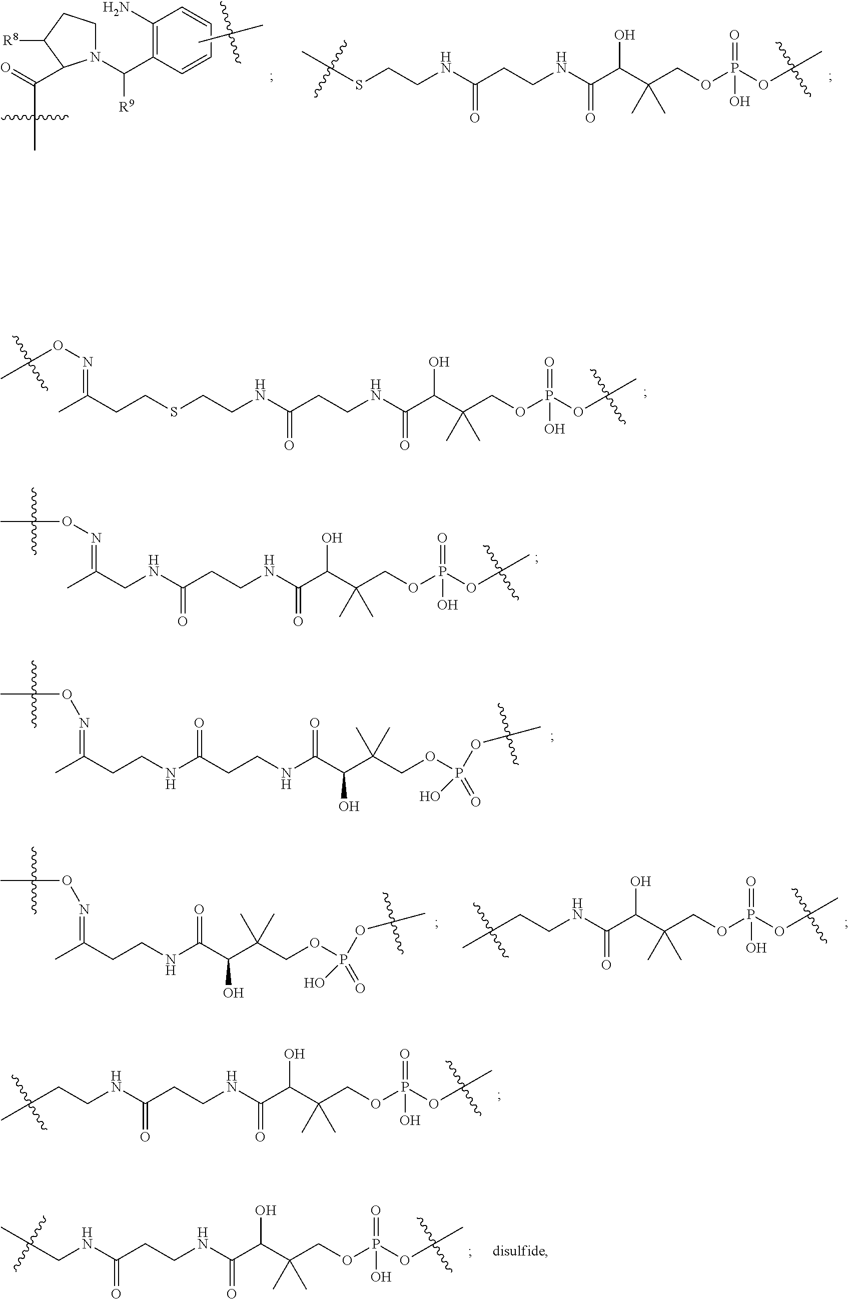
wherein:
R³² is H, C₁0.4 alkyl, phenyl, pyrimidine or pyridine;
R³⁵ is H, C₁₋₆ alkyl, phenyl or C₁0.4 alkyl substituted with 1 to 3 —OH groups;
each R⁷ is independently selected from H, C₁₋₆ alkyl, fluoro, benzyloxy substituted with —C(═O)OH, benzyl substituted with —C(═O)OH, C₁0.4 alkoxy substituted with —C(═O)OH and C₁0.4 alkyl substituted with —C(═O)OH;
R³⁷ is independently selected from H, phenyl and pyridine;
q is 0, 1, 2 or 3;
R⁸ is H or methyl; and
R⁹ is H, —CH₃ or phenyl.

34. The antibody-drug conjugate any one of claims 23 to 33, wherein the peptide group comprises 1 to 4 or 1 to 3 or 1 or 2 amino acid residues, optionally the amino acid residues are selected from L-glycine (Gly), L-valine (Val), L-citrulline (Cit), L-cysteic acid (sulfo-Ala), L-lysine (Lys), L-isoleucine (lie), L-phenylalanine (Phe), L-methionine (Met), L-asparagine (Asn), L-proline (Pro), L-alanine (Ala), L-leucine (Leu), L-tryptophan (Trp), and L-tyrosine (Tyr).

35. The antibody-drug conjugate any one of claims 23 to 33, wherein the peptide group comprises Val-Cit, Phe-Lys, Val-Ala, Val-Lys, Leu-Cit, sulfo-Ala-Val, and/or sulfo-Ala-Val-Ala.

36. The antibody-drug conjugate any one of claims 23 to 35, wherein Lp is selected from:

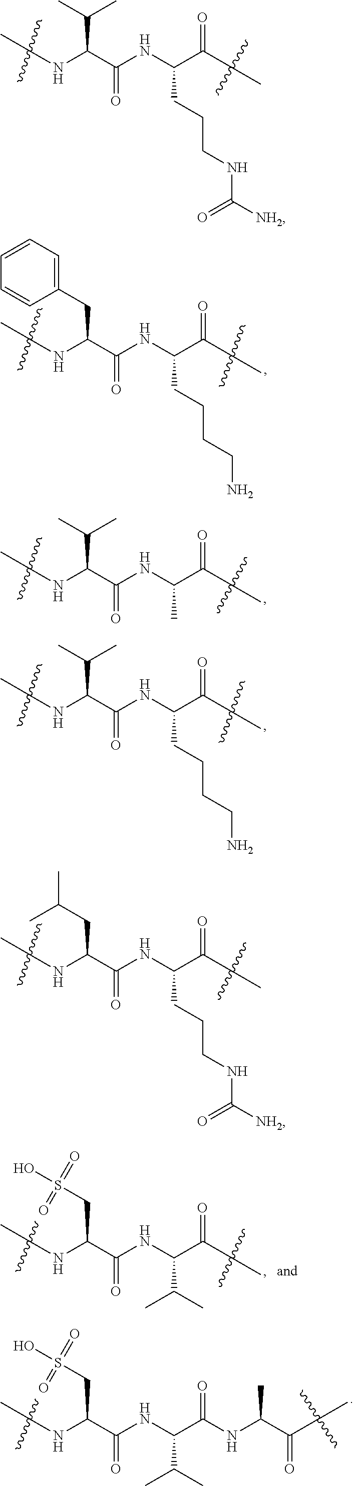

37. The antibody-drug conjugate of any one of claims 23 to 36, wherein:
(L-D) comprises or is formed from a compound of formula: 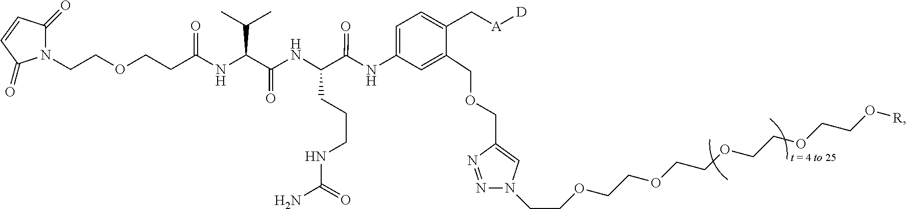
(1) 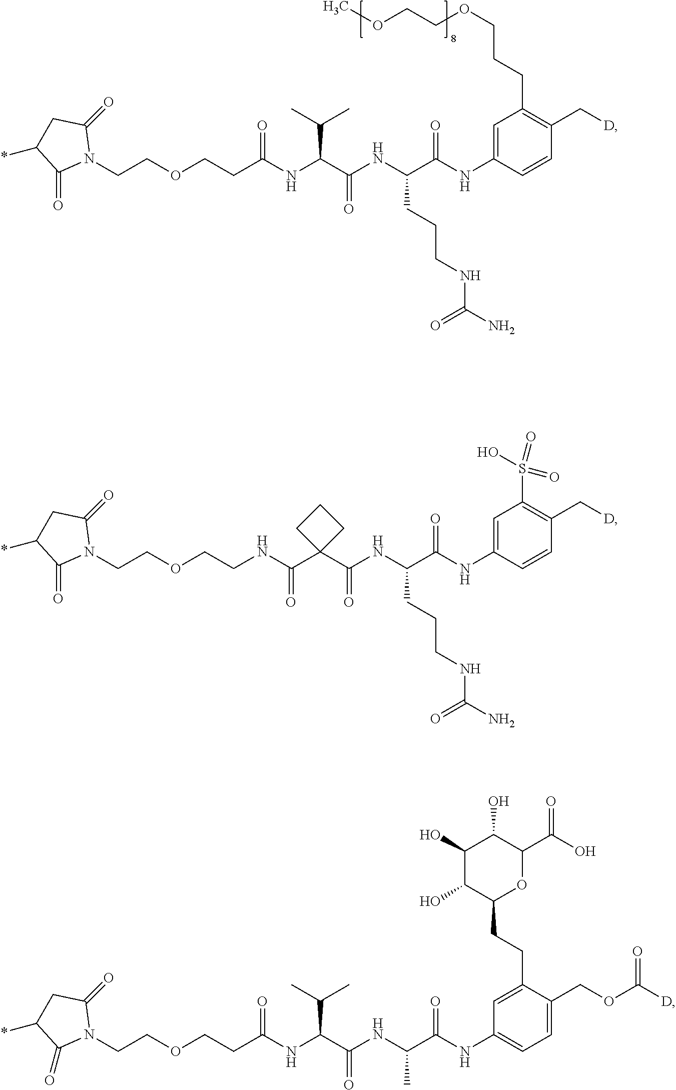 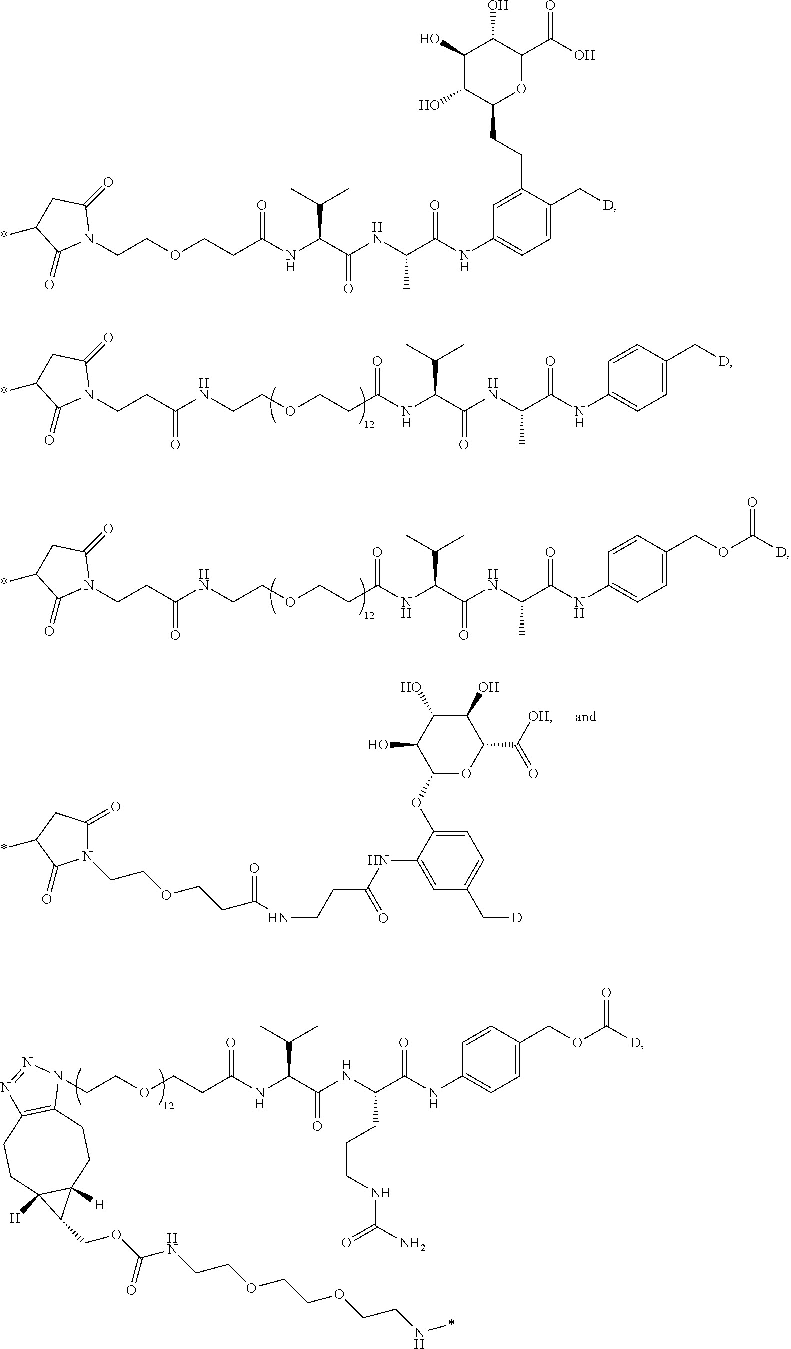
wherein:
R is H, —CH₃ or —CH₂CH₂C(═O)OH;
A is a bond, —OC(═O)—*, 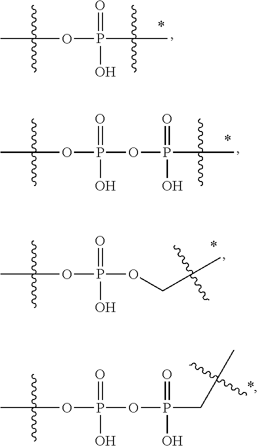 [SMILES: *OP(*)(=O)O ; *OP(=O)(O)OP(*)(=O)O ; *COP(=O)(O)O* ; *CP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a panRAS inhibitor;
(2) 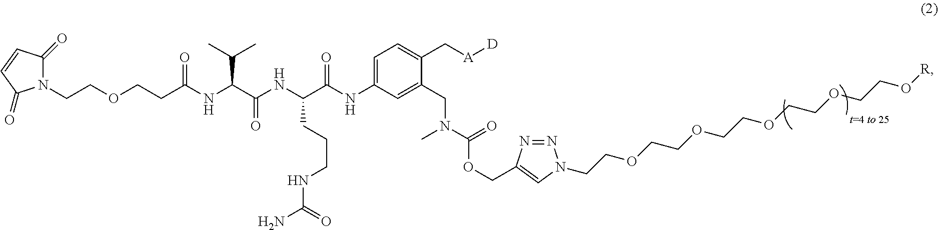
wherein:
R is H, —CH₃ or —CH₂CH₂C(═O)OH;
A is a bond, —OC(═O)—*, 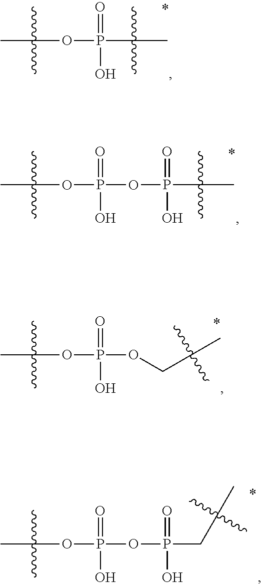 [SMILES: *OP(*)(=O)O ; *OP(=O)(O)OP(*)(=O)O ; *COP(=O)(O)O* ; *CP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a panRAS inhibitor;
(3) 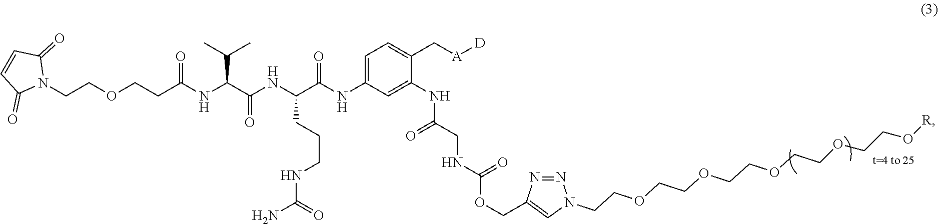
wherein:
R is H, —CH₃ or —CH₂CH₂C(═O)OH;
A is a bond, —OC(═O)—*, 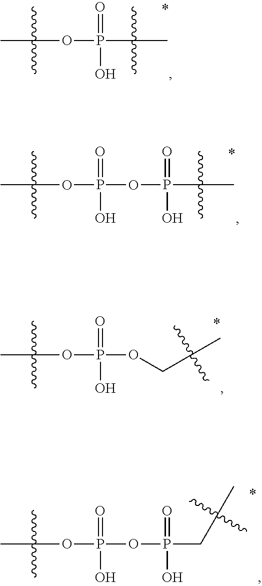 [SMILES: *OP(*)(=O)O ; *OP(=O)(O)OP(*)(=O)O ; *COP(=O)(O)O* ; *CP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a panRAS inhibitor;
(4) 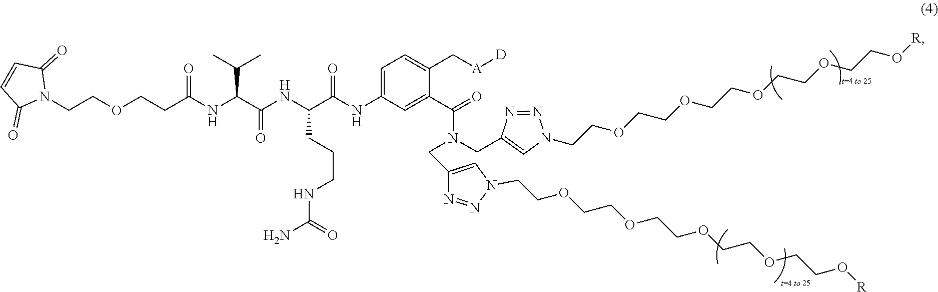
wherein:
each R is independently selected from H, —CH₃, and —CH₂CH₂C(═O)OH;
A is a bond, —OC(═O)—*, 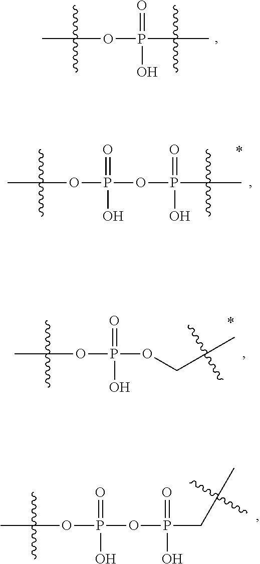 [SMILES: *OP(*)(=O)O ; *OP(=O)(O)OP(*)(=O)O ; *COP(=O)(O)O* ; *CP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a panRAS inhibitor;
(5) 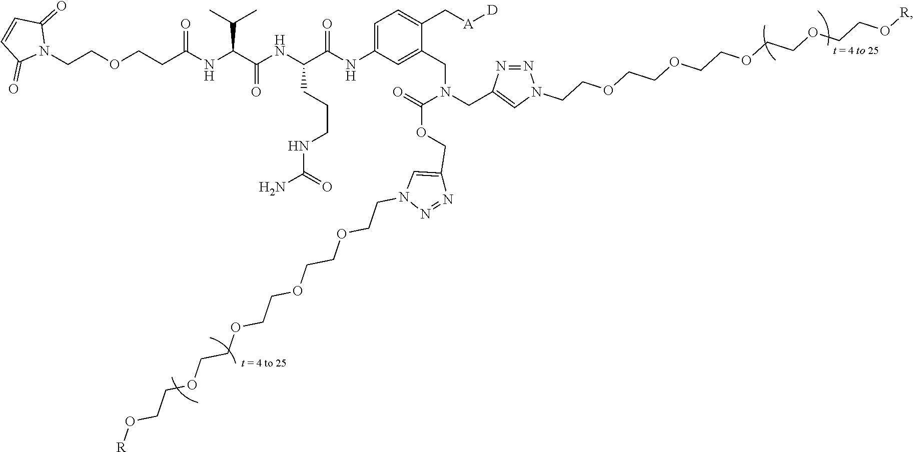
wherein:
each R is independently selected from H, —CH₃, and —CH₂CH₂C(═O)OH;
A is a bond, —OC(═O)—*, 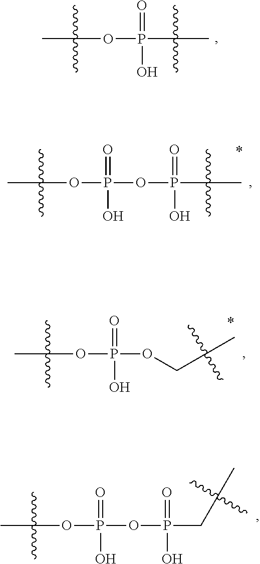 [SMILES: *OP(*)(=O)O ; *OP(=O)(O)OP(*)(=O)O ; *COP(=O)(O)O* ; *CP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a panRAS inhibitor;
(6) 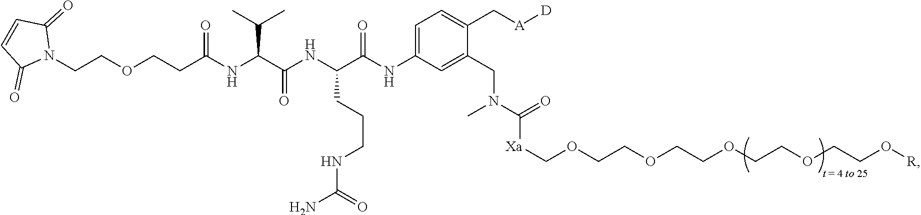
wherein:
Xa is —CH₂—, —OCH₂—, —NHCH₂— or —NRCH₂— and each R independently is H, —CH₃ or —CH₂CH₂C(═O)OH;
A is a bond, —OC(═O)—*, 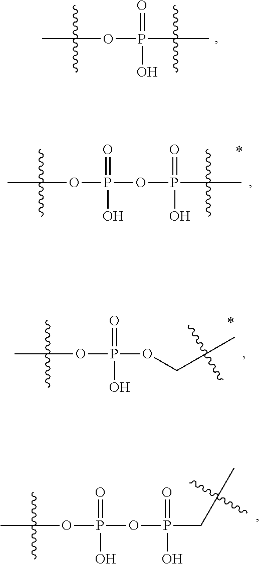 [SMILES: *OP(*)(=O)O ; *OP(=O)(O)OP(*)(=O)O ; *COP(=O)(O)O* ; *CP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a panRAS inhibitor;
(7) 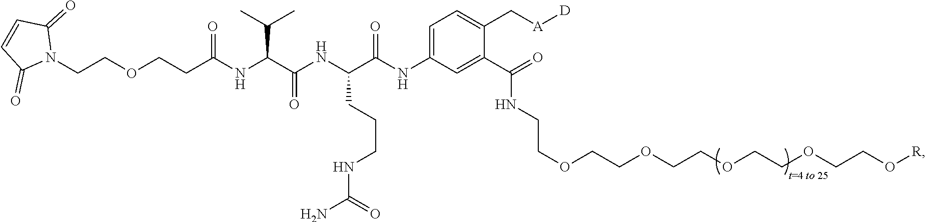
wherein:
R is H, —CH₃ or —CH₂CH₂C(═O)OH;
A is a bond, OC(═O)—*, 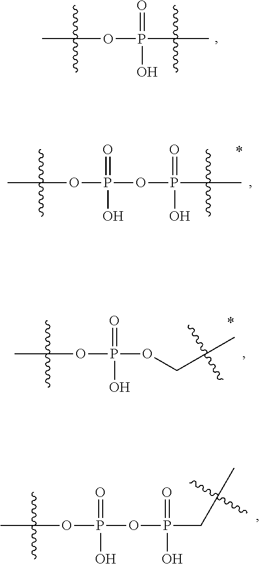 [SMILES: *OP(*)(=O)O ; *OP(=O)(O)OP(*)(=O)O ; *COP(=O)(O)O* ; *CP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a panRAS inhibitor;
(8) 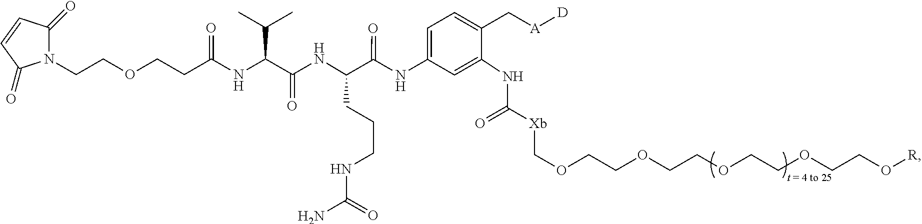
wherein:
Xb is —CH₂—, —OCH₂—, —NHCH₂— or —NRCH₂— and each R independently is H, —CH₃ or —CH₂CH₂C(═O)OH;
A is a bond, —OC(═O)—*, 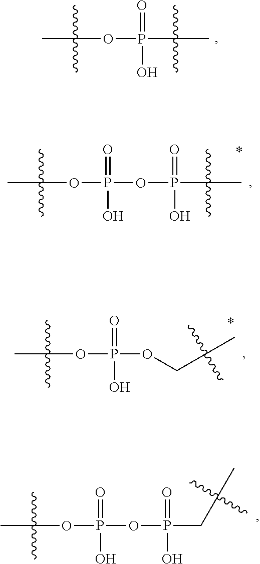 [SMILES: *OP(*)(=O)O ; *OP(=O)(O)OP(*)(=O)O ; *COP(=O)(O)O* ; *CP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a panRAS inhibitor;
(9) 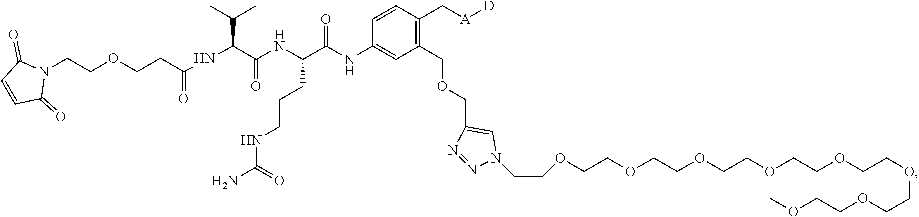
wherein:
A is a bond, —OC(═O)—*, 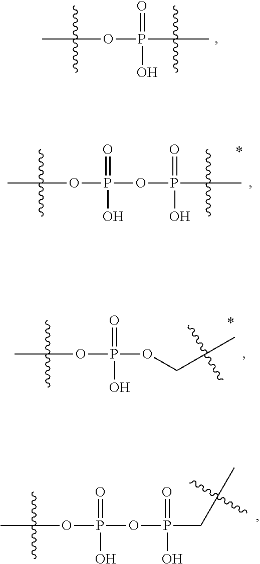 [SMILES: *OP(*)(=O)O ; *OP(=O)(O)OP(*)(=O)O ; *COP(=O)(O)O* ; *CP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a panRAS inhibitor;
(10) 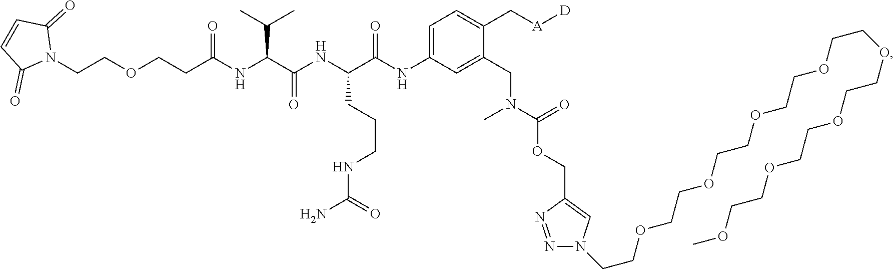
A is a bond, —OC(═O)—*, 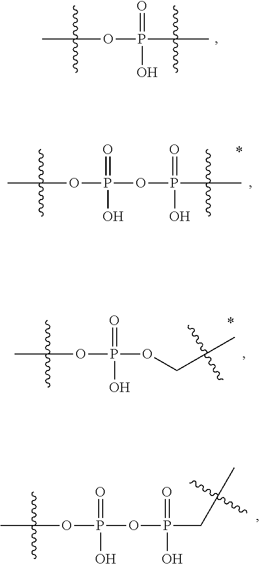 [SMILES: *OP(*)(=O)O ; *OP(=O)(O)OP(*)(=O)O ; *COP(=O)(O)O* ; *CP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a panRAS inhibitor;
(11), 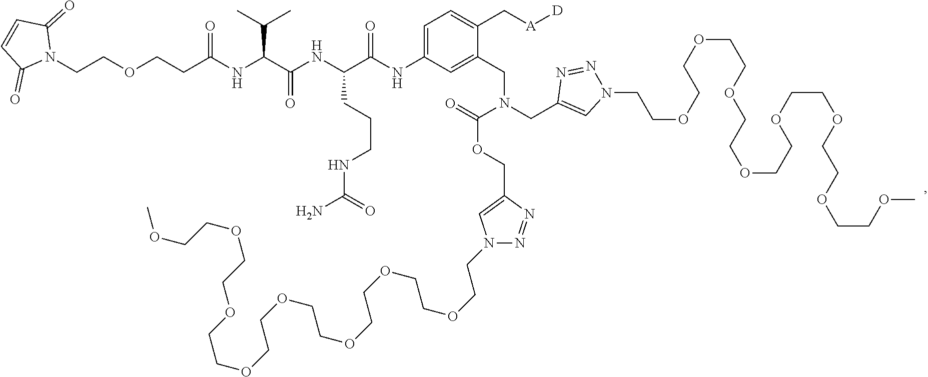
wherein:
A is a bond, —OC(═O)—*, 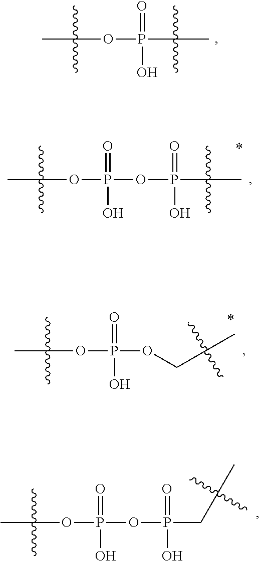 [SMILES: *OP(*)(=O)O ; *OP(=O)(O)OP(*)(=O)O ; *COP(=O)(O)O* ; *CP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a panRAS inhibitor;
(12) 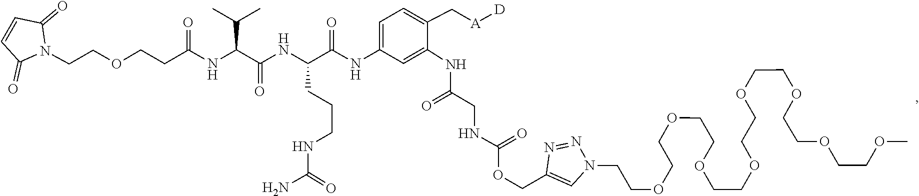
wherein:
A is a bond, —OC(═O)—*, 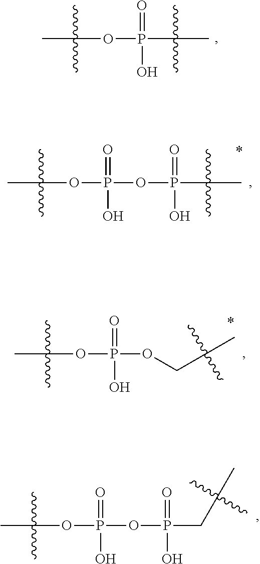 [SMILES: *OP(*)(=O)O ; *OP(=O)(O)OP(*)(=O)O ; *COP(=O)(O)O* ; *CP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a panRAS inhibitor;
(13) 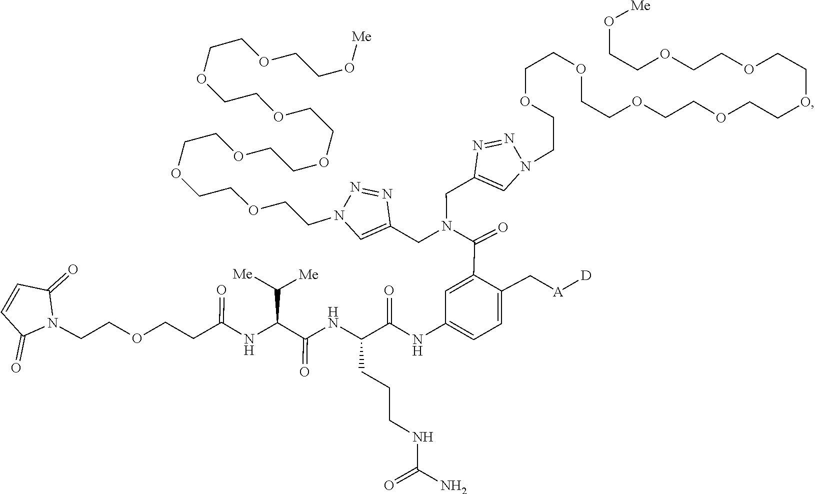
wherein:
A is a bond, —OC(═O)—*, 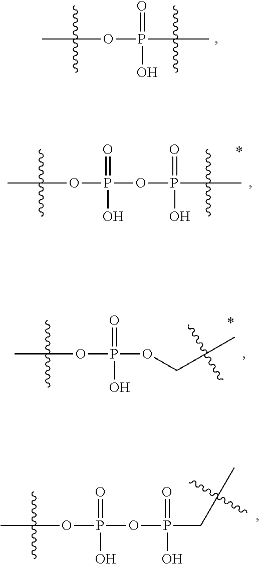 [SMILES: *OP(*)(=O)O ; *OP(=O)(O)OP(*)(=O)O ; *COP(=O)(O)O* ; *CP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a panRAS inhibitor;
(14) 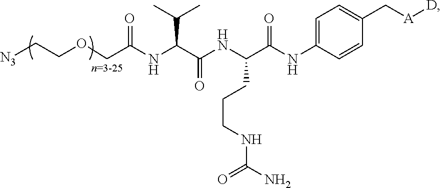
A is a bond, —OC(═O)—*, 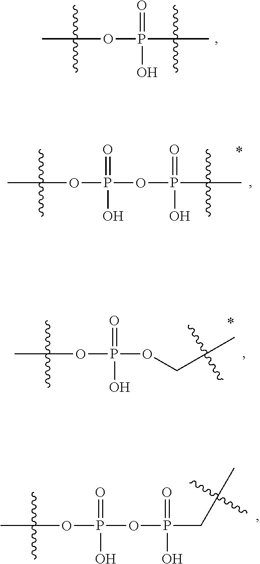 [SMILES: *OP(*)(=O)O ; *OP(=O)(O)OP(*)(=O)O ; *COP(=O)(O)O* ; *CP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a panRAS inhibitor;
(15) 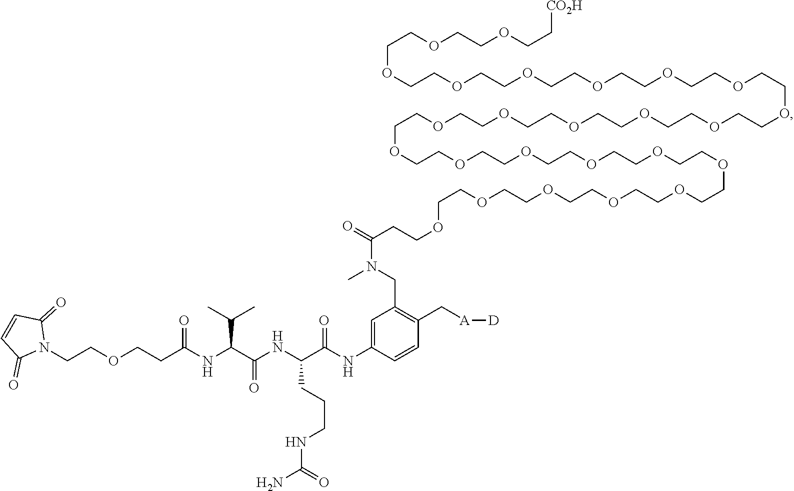
wherein:
A is a bond, —OC(_O)-*, 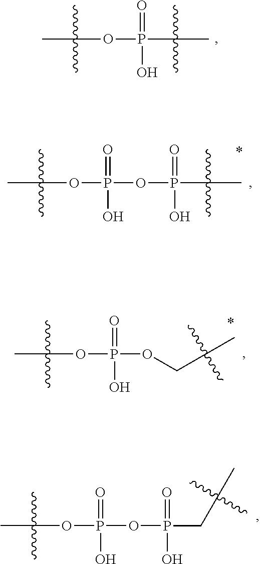 [SMILES: *OP(*)(=O)O ; *OP(=O)(O)OP(*)(=O)O ; *COP(=O)(O)O* ; *CP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)H*,
wherein each R^{a} is independently selected from H, C₁-C₆ alkyl, and C₃-C₈ cycloalkyl and the * of A indicates the point of attachment to D; and
D is a panRAS inhibitor; or
(16) 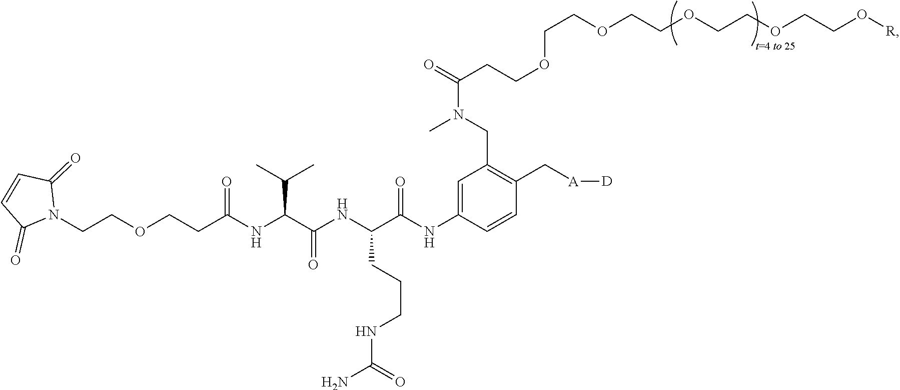
wherein:
each R independently is H, —CH₃ or —CH₂CH₂C(═O)OH;
A is a bond, —OC(═O)—*, 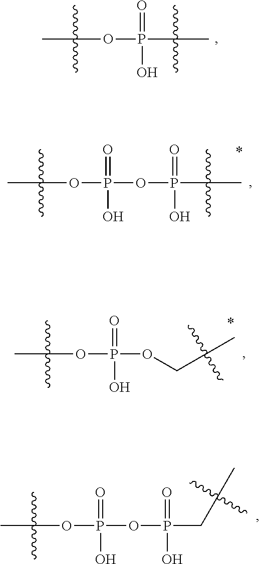 [SMILES: *OP(*)(=O)O ; *OP(=O)(O)OP(*)(=O)O ; *COP(=O)(O)O* ; *CP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₈ alkyl, and C₃-C₈ cycloalkyl and the * of A indicates the point of attachment to D; and
D is a panRAS inhibitor, or
(17) 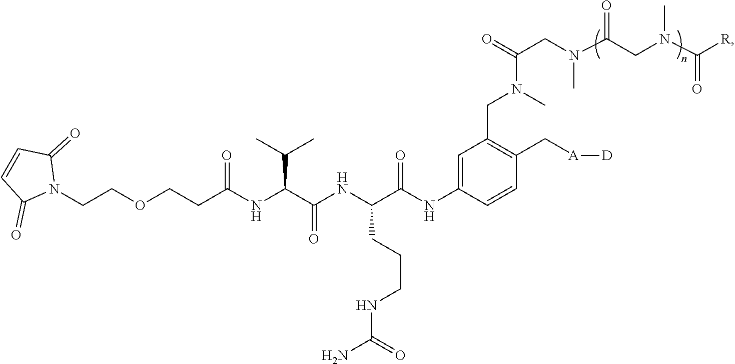
wherein:
each R independently is H, —CH₃ or —CH₂CH₂C(═O)OH;
A is a bond, —OC(═O)—*, 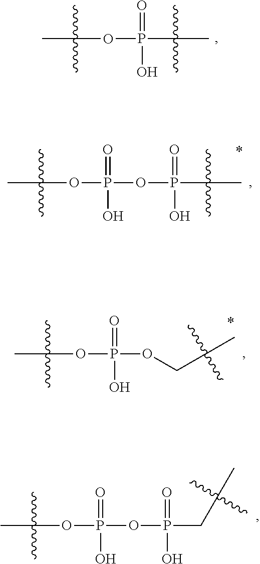 [SMILES: *OP(*)(=O)O ; *OP(=O)(O)OP(*)(=O)O ; *COP(=O)(O)O* ; *CP(=O)(O)OP(=O)(O)O*]
—OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₈ alkyl, and C₃-C₈ cycloalkyl and the * of A indicates the point of attachment to D;
n is an integer between 2 and 24; and
D is a PanRAS inhibitor, or
(18) 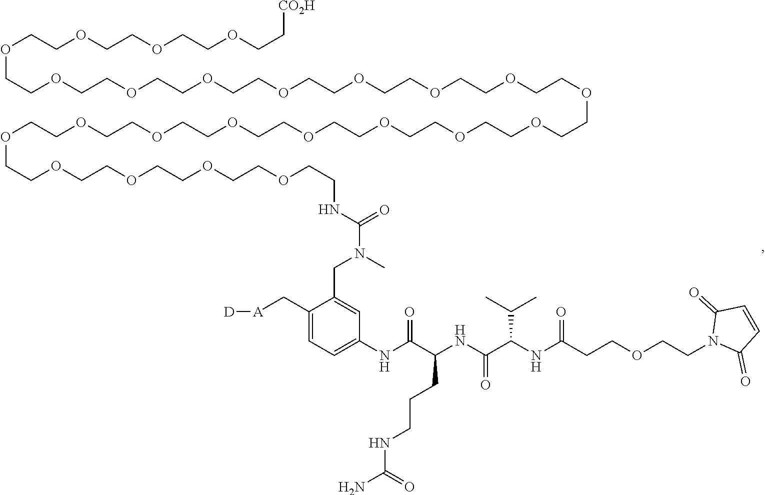
wherein:
A is a bond, —OC(═O)—*, 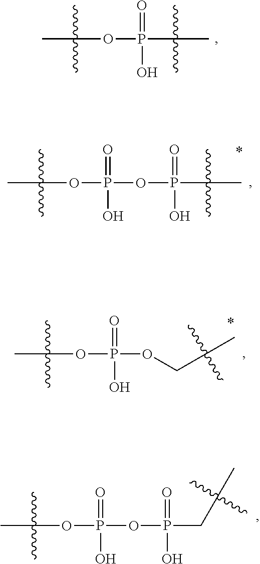 [SMILES: *OP(*)(=O)O ; *OP(=O)(O)OP(*)(=O)O ; *COP(=O)(O)O* ; *CP(=O)(O)OP(=O)(O)O*]
OC(═O)N(CH₃)CH₂CH₂N(CH₃)C(═O)—* or —OC(═O)N(CH₃)C(R^{a})₂C(R^{a})₂N(CH₃)C(═O)—*,
wherein each R^{a} is independently selected from H, C₁-C₆ alkyl, and C₃-C₈ cycloalkyl and the * of A indicates the point of attachment to D; and
D is a panRAS inhibitor.

38. The antibody-drug conjugate of any one of claims 23 to 37, wherein A is a bond and/or R is —CH₃ or —CH₂CH₂COOH.

39. The antibody-drug conjugate of any one of claims 23 to 37, wherein A is —OC(═O)—* and/or R is —CH₃ or —CH₂CH₂COOH.

40. The antibody-drug conjugate of any one of claims 23-39, wherein -(L-D) is formed from a compound selected from:

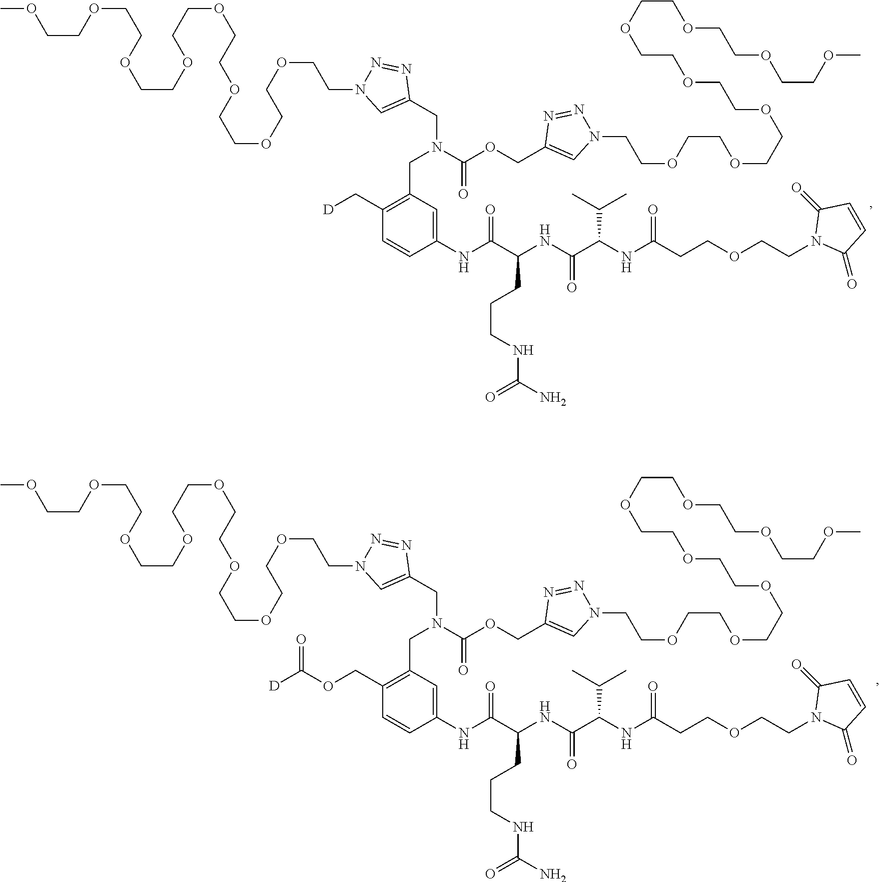 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(Cc1cn(CCOCCOCCOCCOCCOCCOCCOCCOC)nn1)C(=O)OCc1cn(CCOCCOCCOCCOCCOCCOCCOCCOC)nn1 ; [2H]C(=O)OCc1ccc(NC(=O)[C@H](CCCNC(N)=O)NC(=O)[C@@H](NC(=O)CCOCCN2C(=O)C=CC2=O)C(C)C)cc1CN(Cc1cn(CCOCCOCCOCCOCCOCCOCCOCCOC)nn1)C(=O)OCc1cn(CCOCCOCCOCCOCCOCCOCCOCCOC)nn1]

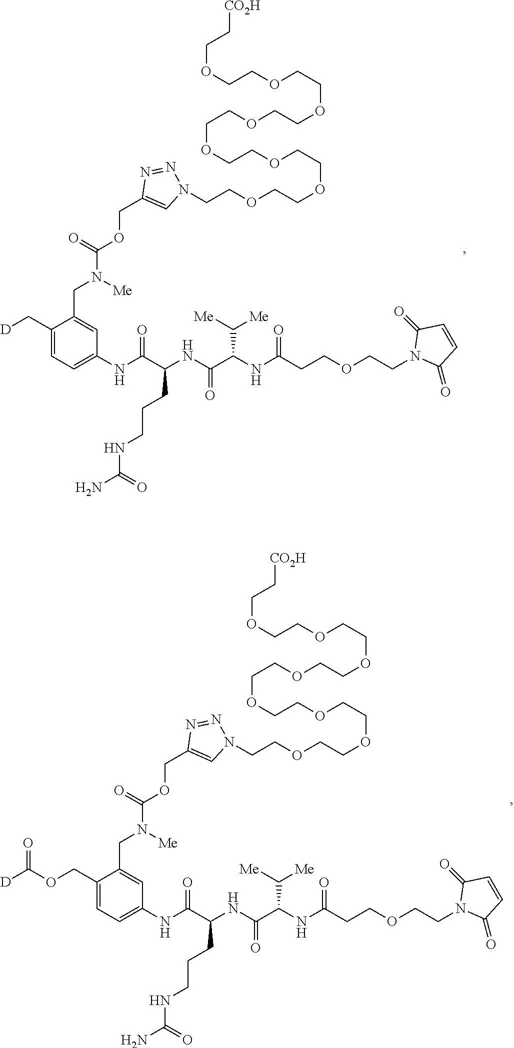 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)OCc1cn(CCOCCOCCOCCOCCOCCOCCOCCOCCC(=O)O)nn1 ; [2H]C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)OCc1cn(CCOCCOCCOCCOCCOCCOCCOCCOCCC(=O)O)nn1]

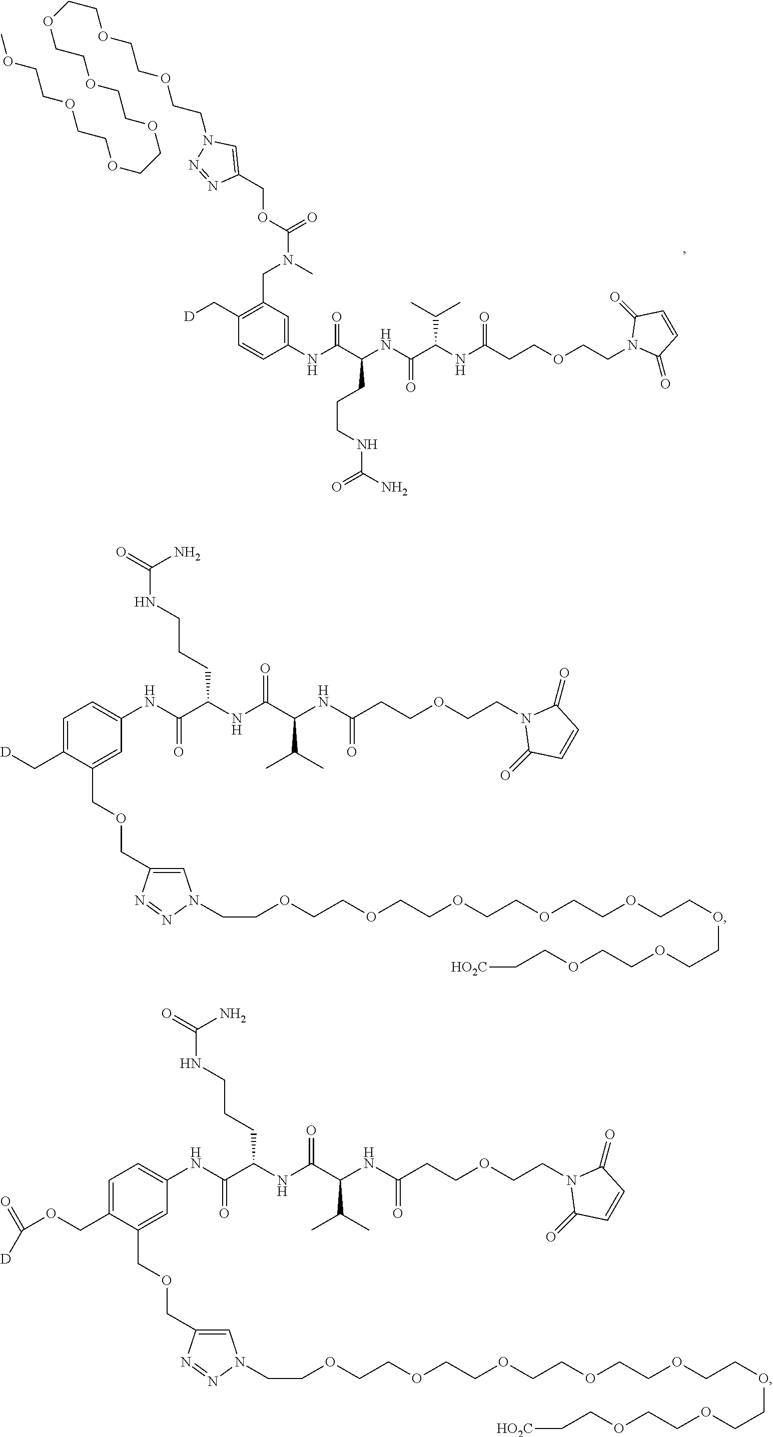 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)OCc1cn(CCOCCOCCOCCOCCOCCOCCOCCOC)nn1 ; [2H]Cc1ccc(NC(=O)[C@H](CCCNC(N)=O)NC(=O)[C@@H](NC(=O)CCOCCN2C(=O)C=CC2=O)C(C)C)cc1COCc1cn(CCOCCOCCOCCOCCOCCOCCOCCOCCC(=O)O)nn1 ; [2H]C(=O)OCc1ccc(NC(=O)[C@H](CCCNC(N)=O)NC(=O)[C@@H](NC(=O)CCOCCN2C(=O)C=CC2=O)C(C)C)cc1COCc1cn(CCOCCOCCOCCOCCOCCOCCOCCOCCC(=O)O)nn1]

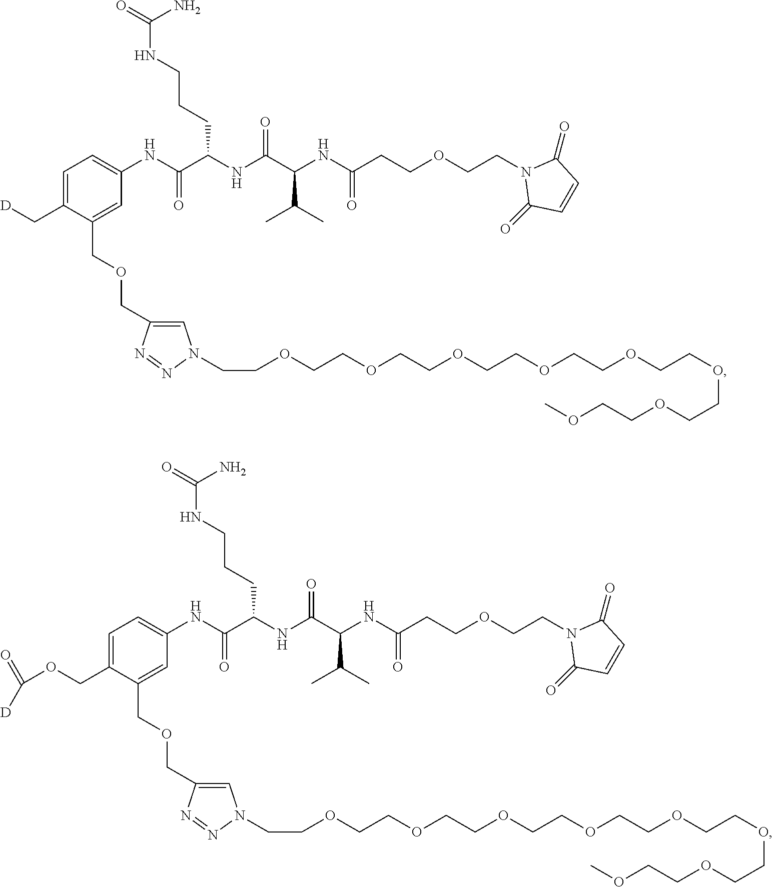 [SMILES: [2H]Cc1ccc(NC(=O)[C@H](CCCNC(N)=O)NC(=O)[C@@H](NC(=O)CCOCCN2C(=O)C=CC2=O)C(C)C)cc1COCc1cn(CCOCCOCCOCCOCCOCCOCCOCCOC)nn1 ; [2H]C(=O)OCc1ccc(NC(=O)[C@H](CCCNC(N)=O)NC(=O)[C@@H](NC(=O)CCOCCN2C(=O)C=CC2=O)C(C)C)cc1COCc1cn(CCOCCOCCOCCOCCOCCOCCOCCOC)nn1]

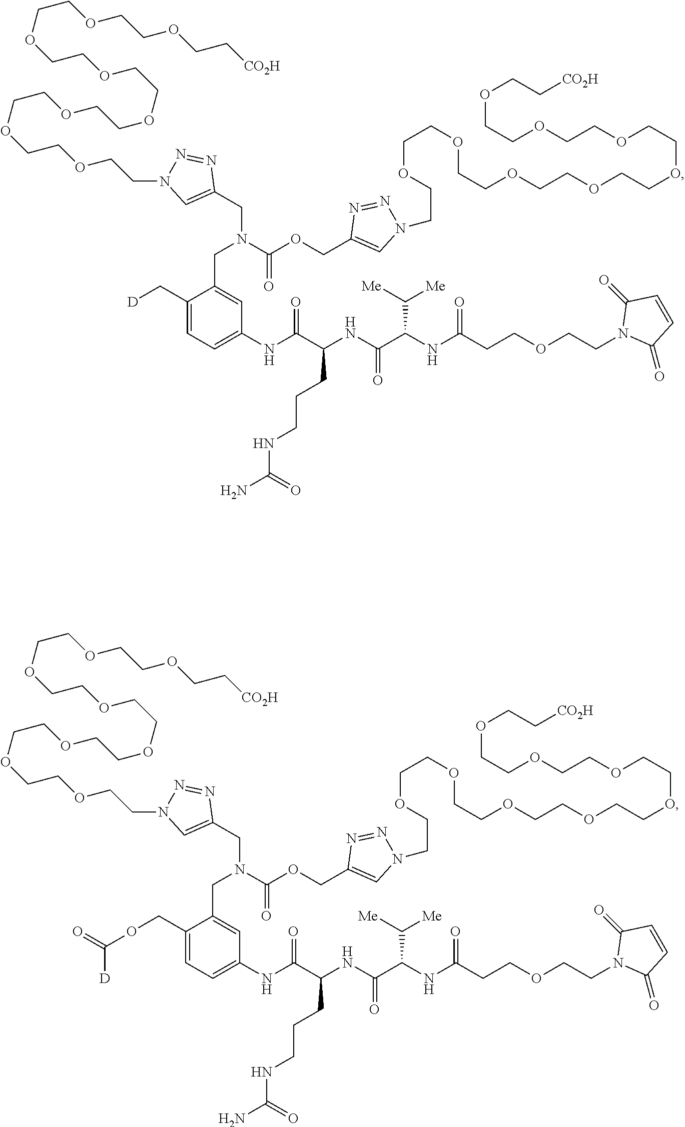 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(Cc1cn(CCOCCOCCOCCOCCOCCOCCOCCOCCC(=O)O)nn1)C(=O)OCc1cn(CCOCCOCCOCCOCCOCCOCCOCCOCCC(=O)O)nn1 ; [2H]C(=O)OCc1ccc(NC(=O)[C@H](CCCNC(N)=O)NC(=O)[C@@H](NC(=O)CCOCCN2C(=O)C=CC2=O)C(C)C)cc1CN(Cc1cn(CCOCCOCCOCCOCCOCCOCCOCCOCCC(=O)O)nn1)C(=O)OCc1cn(CCOCCOCCOCCOCCOCCOCCOCCOCCC(=O)O)nn1]

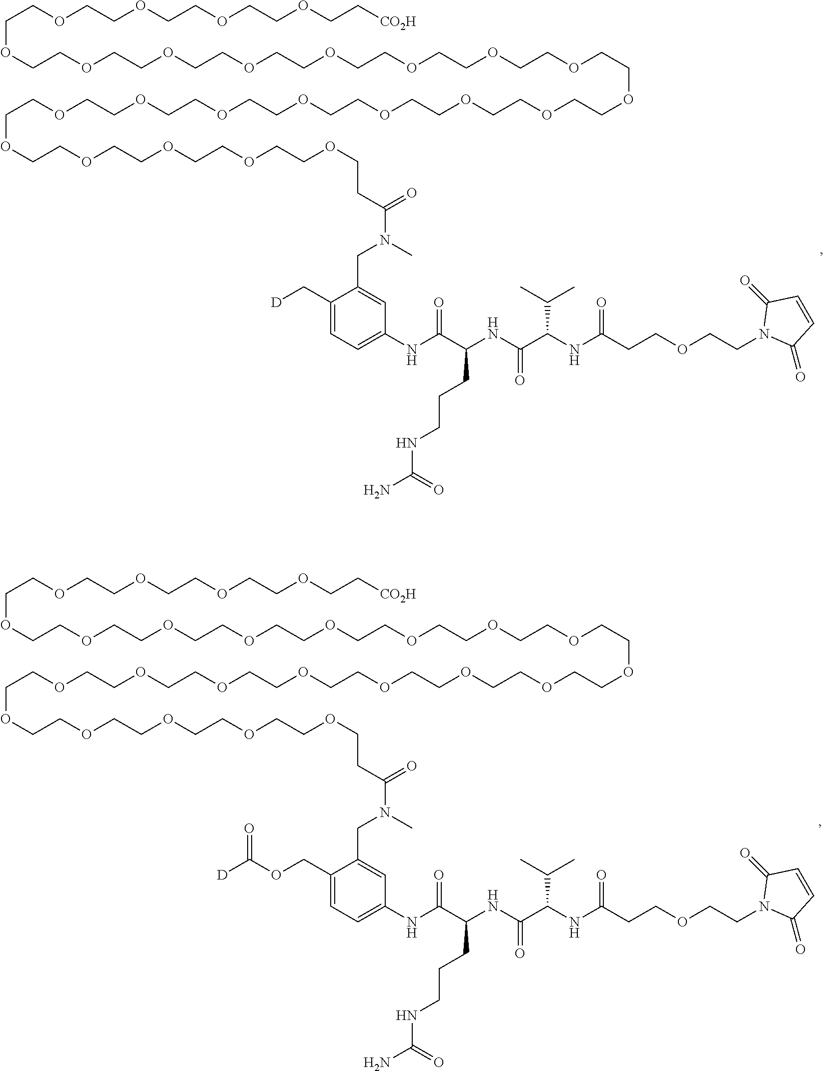 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O ; [2H]C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCOCCOCCOCCOCCOCCOCCOCCOCCOCCOCCOCCOCCOCCOCCOCCOCCOCCOCCOCCOCCOCCOCCOCCC(=O)O]

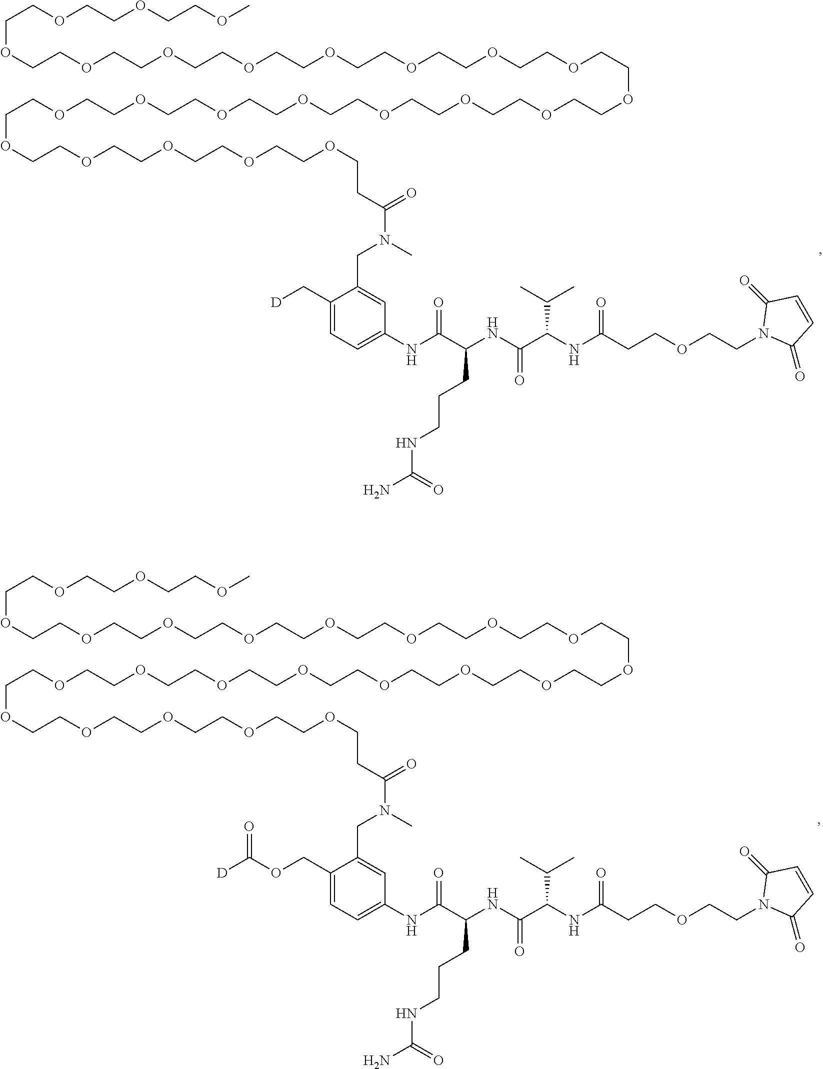 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCOCCOCCOCCOCCOCCOCCOCCOCCOCCOCCOCCOCCOCCOCCOCCOCCOCCOCCOCCOCCOCCOC ; [2H]C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CCOCCOCCOCCOCCOCCOCCOCCOCCOCCOCCOCCOCCOCCOCCOCCOCCOCCOCCOCCOCCOCCOCCOCCOC]

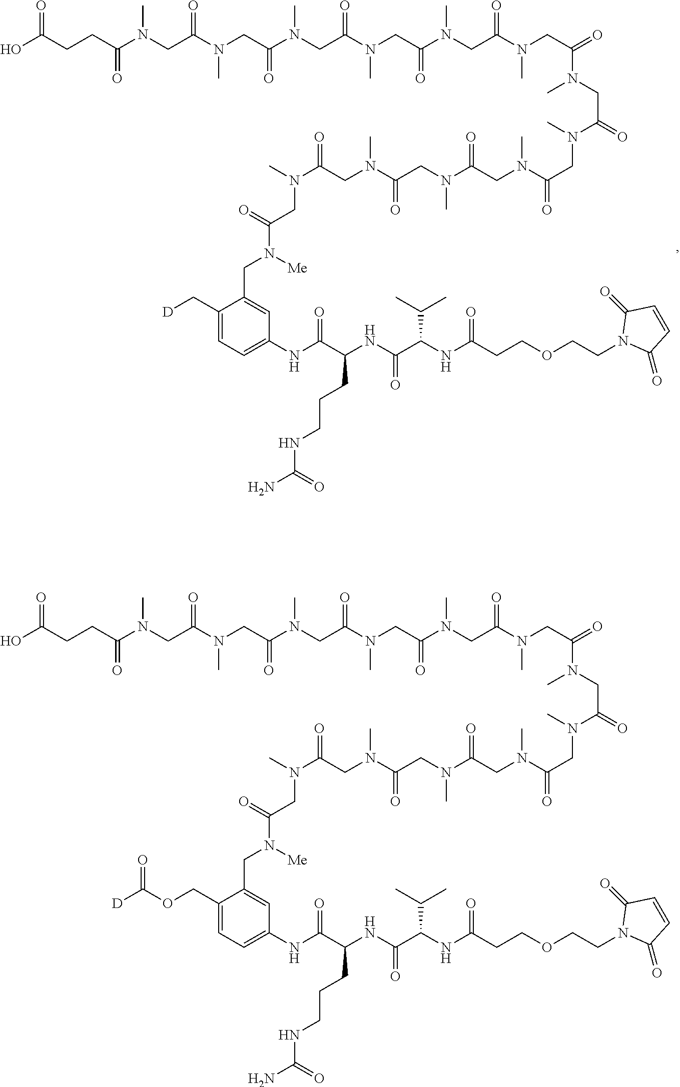 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O ; [2H]C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O]

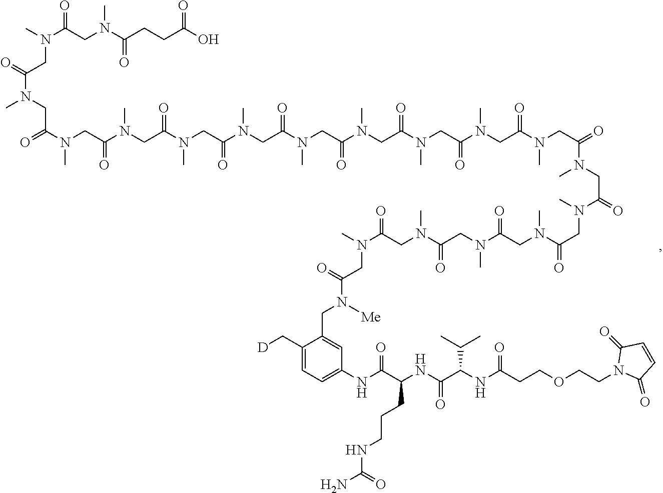 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CCC(=O)O]

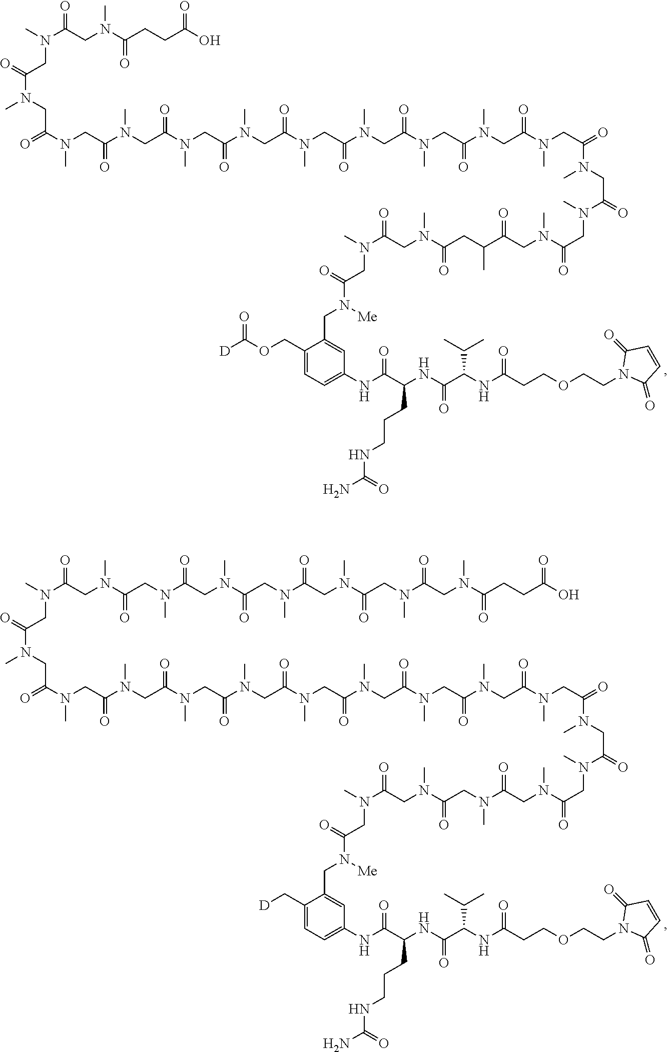

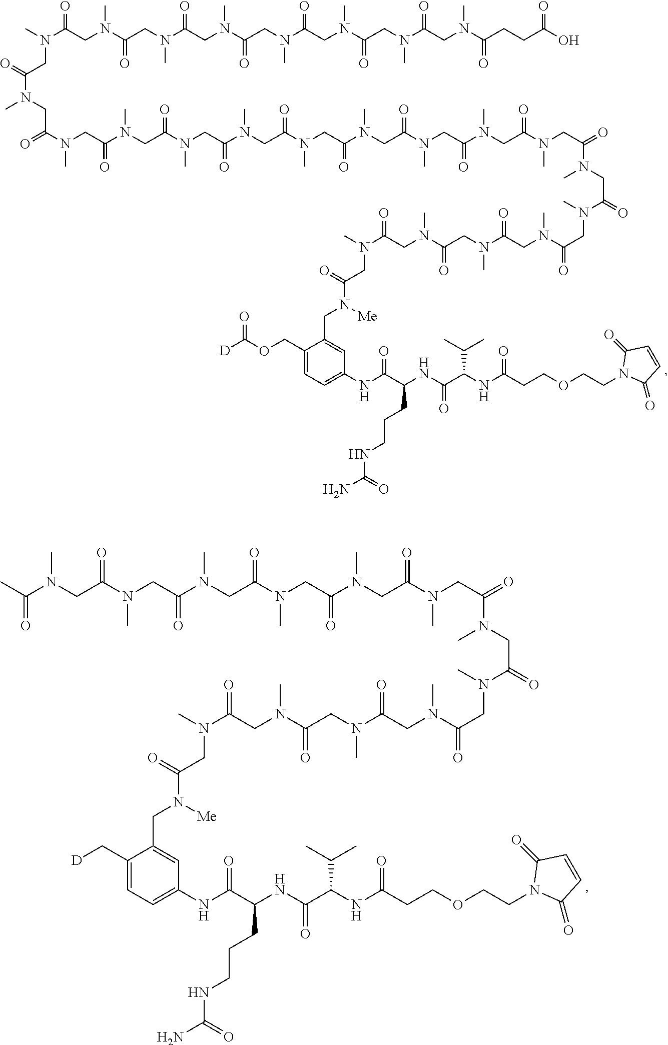

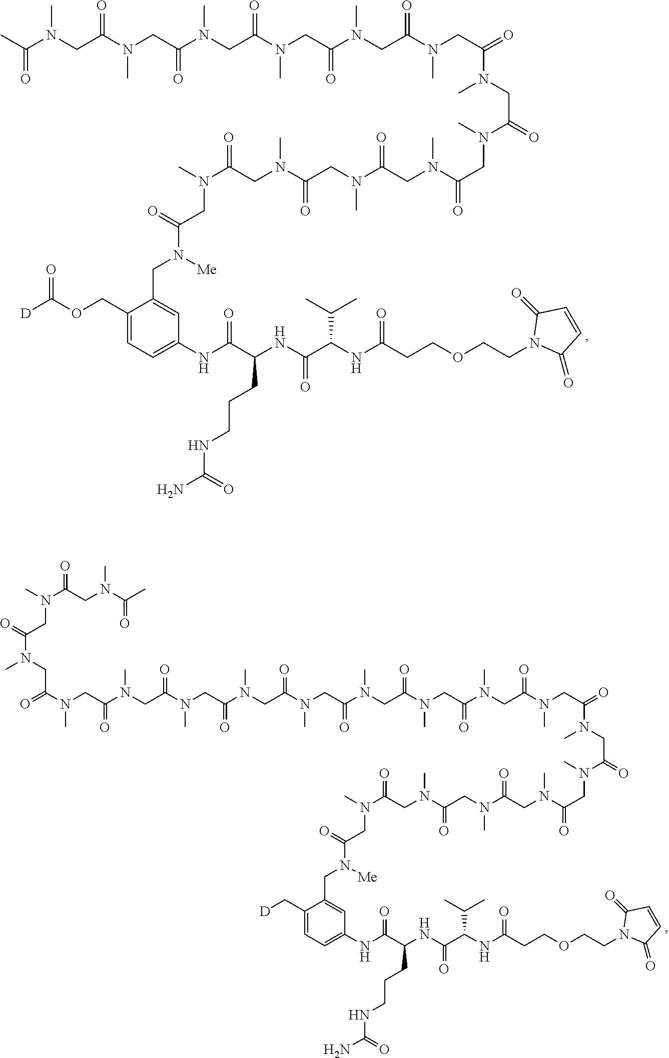 [SMILES: [2H]C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O ; [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(=O)CN(C)C(C)=O]

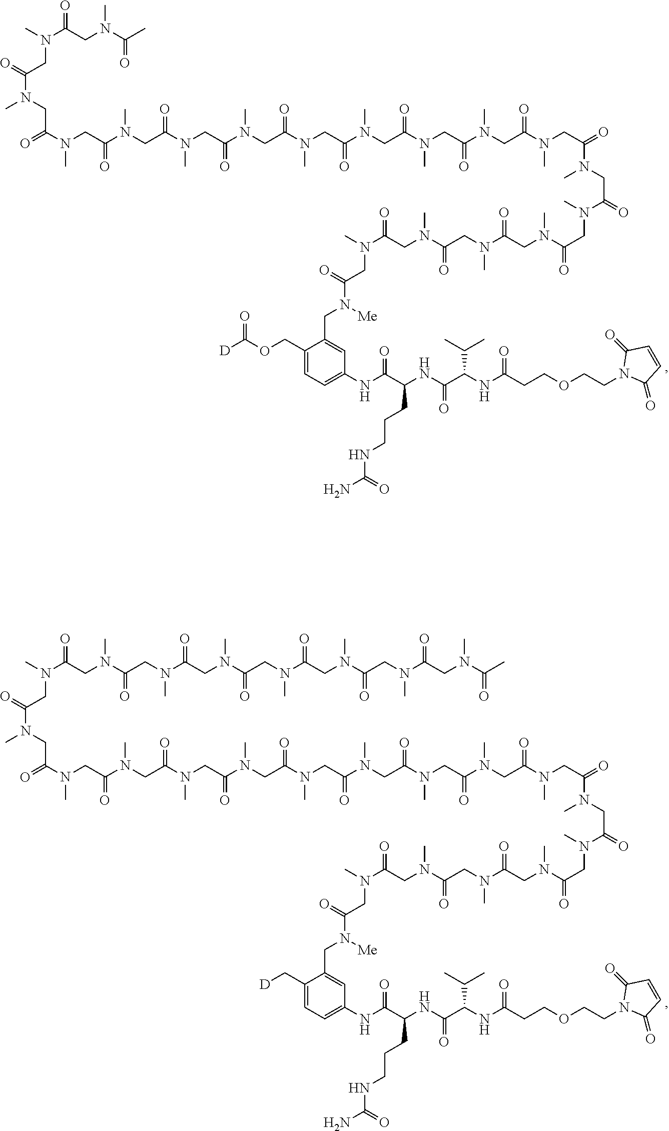

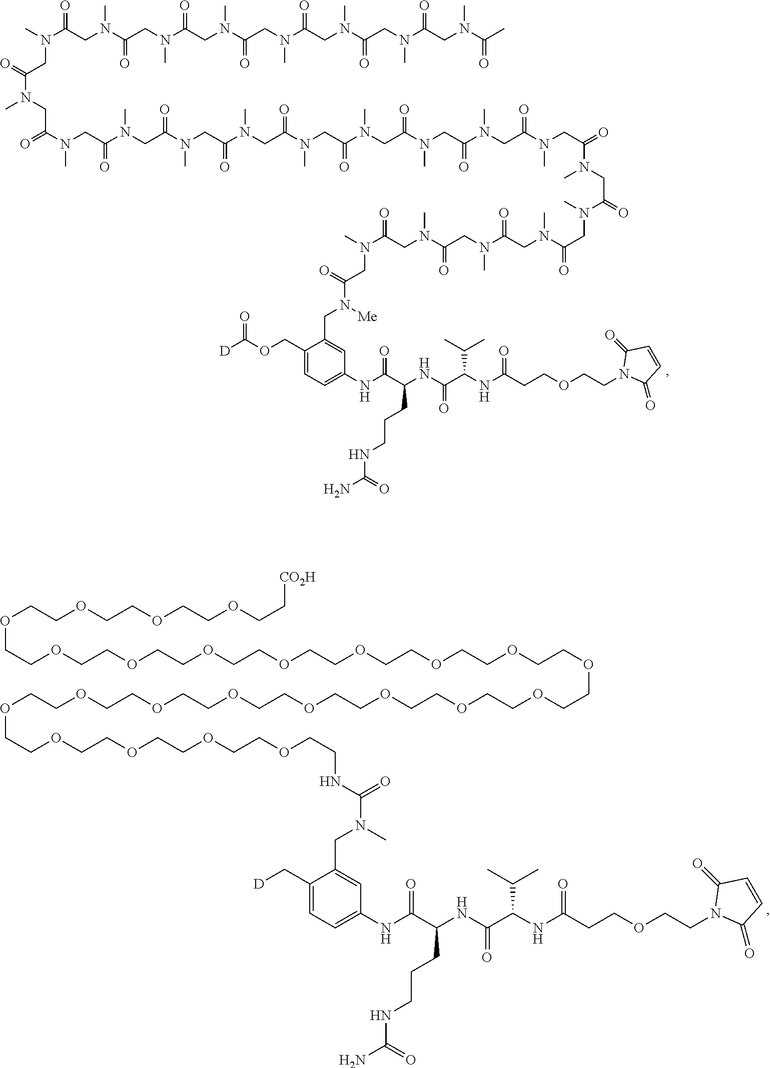

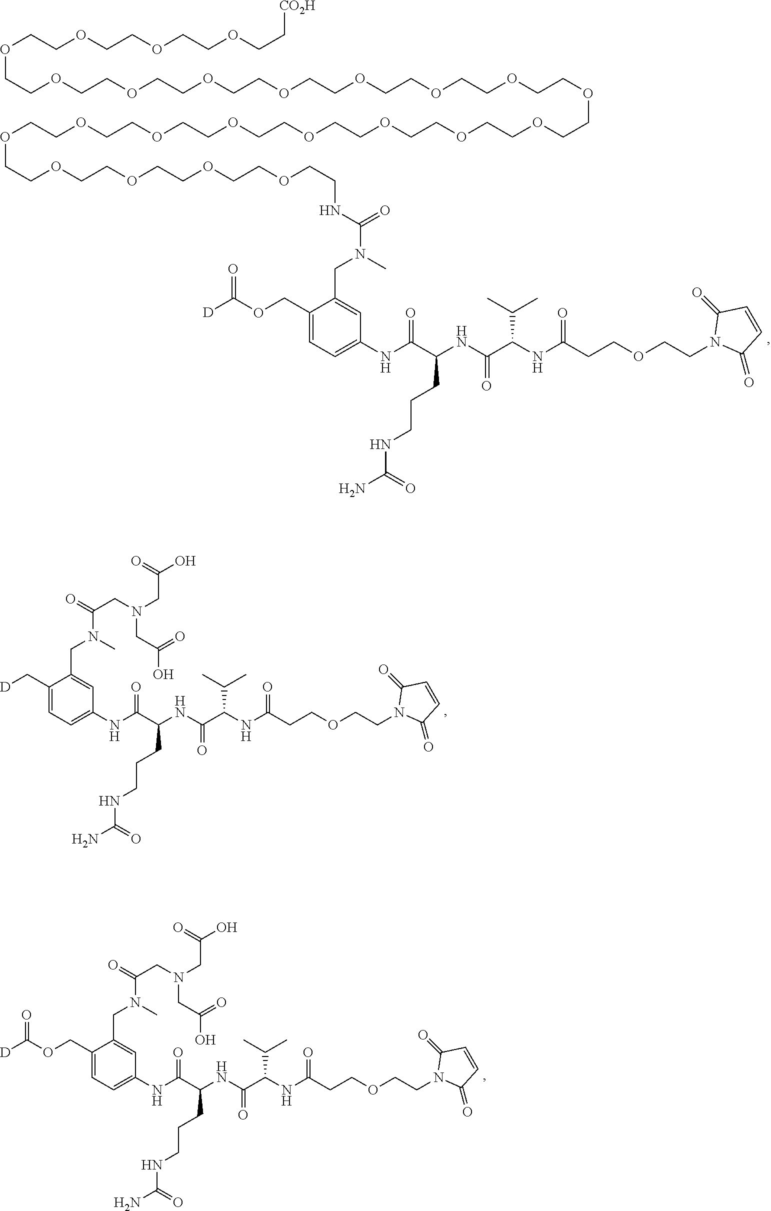 [SMILES: [2H]C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)NCCOCCOCCOCCOCCOCCOCCOCCOCCOCCOCCOCCOCCOCCOCCOCCOCCOCCOCCOCCOCCOCCOCCOCCOCCC(=O)O ; [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)CN(CC(=O)O)CC(=O)O ; [2H]C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)CN(CC(=O)O)CC(=O)O]

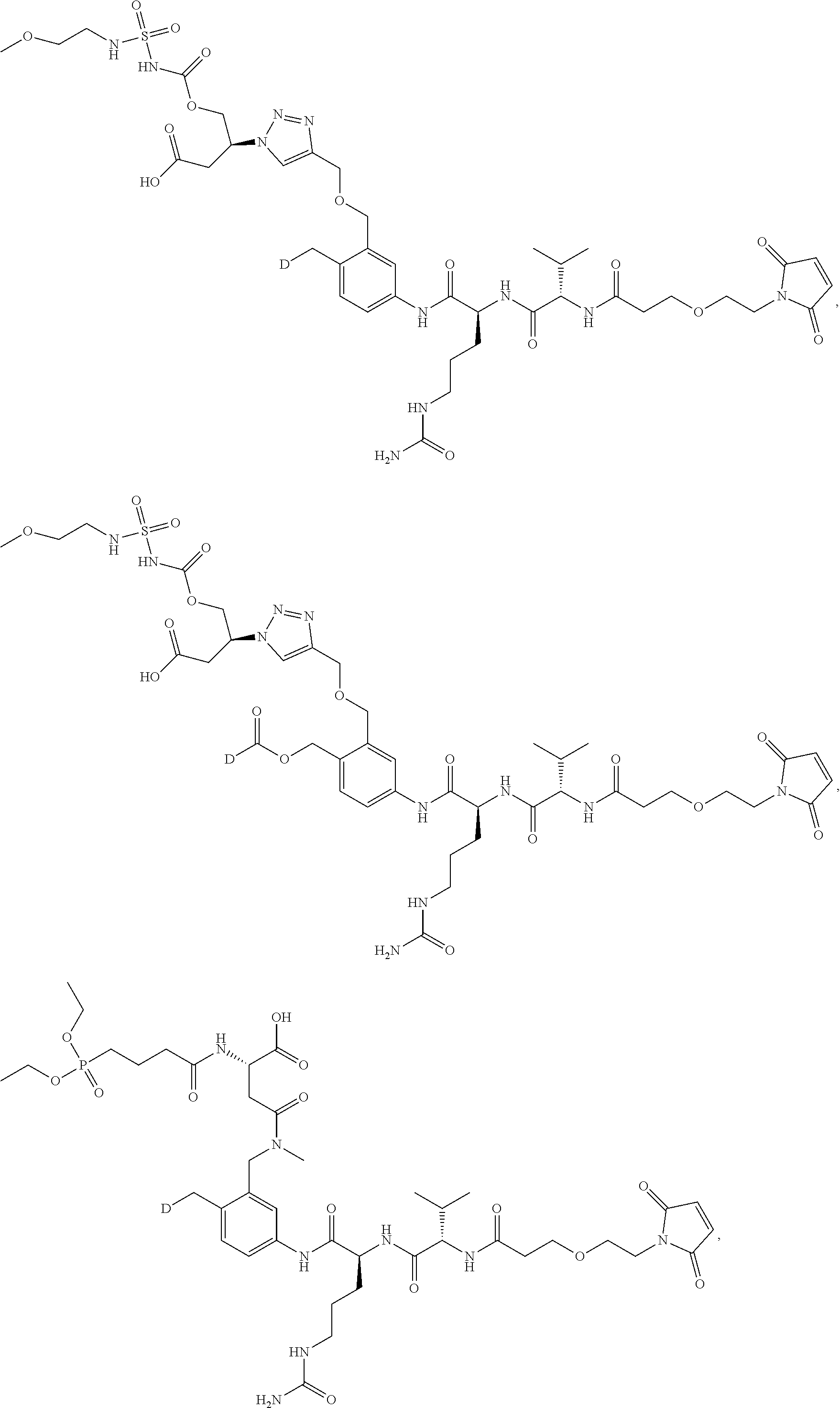 [SMILES: [2H]Cc1ccc(NC(=O)[C@H](CCCNC(N)=O)NC(=O)[C@@H](NC(=O)CCOCCN2C(=O)C=CC2=O)C(C)C)cc1COCc1cn([C@H](COC(=O)NS(=O)(=O)NCCOC)CC(=O)O)nn1 ; [2H]C(=O)OCc1ccc(NC(=O)[C@H](CCCNC(N)=O)NC(=O)[C@@H](NC(=O)CCOCCN2C(=O)C=CC2=O)C(C)C)cc1COCc1cn([C@H](COC(=O)NS(=O)(=O)NCCOC)CC(=O)O)nn1 ; [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)C[C@H](NC(=O)CCCP(=O)(OCC)OCC)C(=O)O]

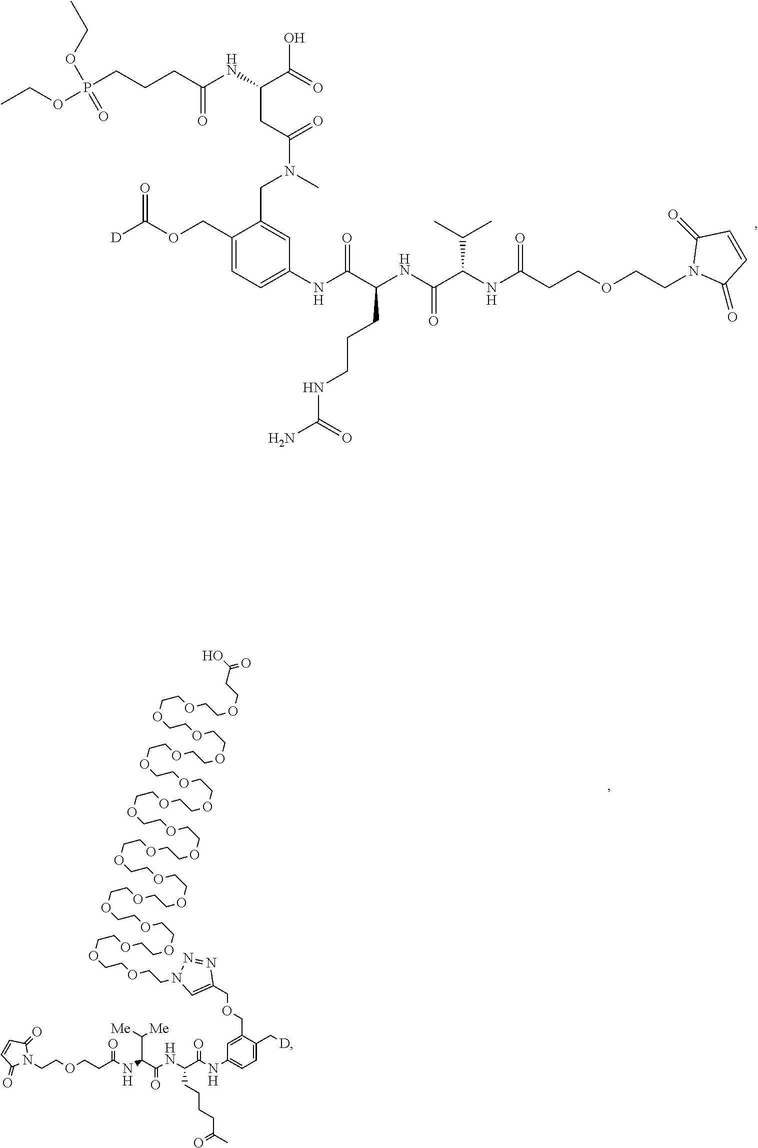

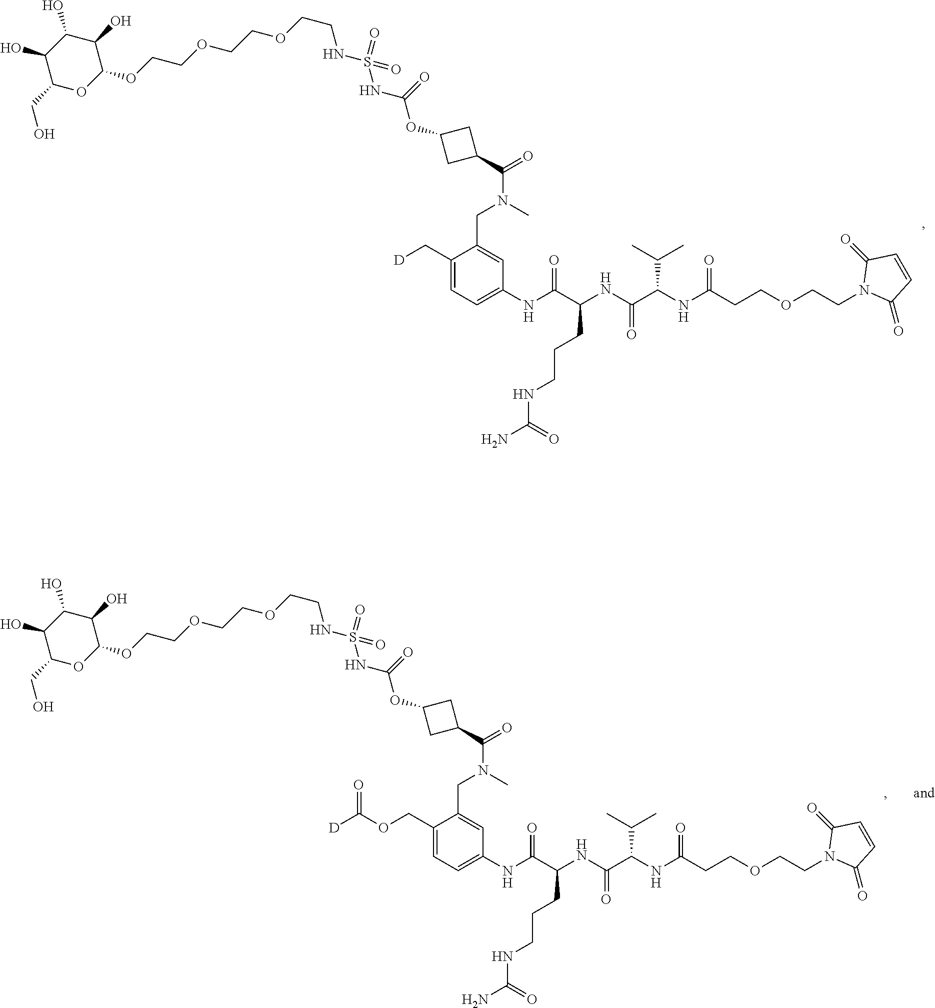 [SMILES: [2H]Cc1ccc(NC(=O)[C@H](CCCNC(N)=O)NC(=O)[C@@H](NC(=O)CCOCCN2C(=O)C=CC2=O)C(C)C)cc1CN(C)C(=O)[C@H]1C[C@H](OC(=O)NS(=O)(=O)NCCOCCOCCO[C@@H]2O[C@H](CO)[C@@H](O)[C@H](O)[C@H]2O)C1 ; [2H]C(=O)OCc1ccc(NC(=O)[C@H](CCCNC(N)=O)NC(=O)[C@@H](NC(=O)CCOCCN2C(=O)C=CC2=O)C(C)C)cc1CN(C)C(=O)[C@H]1C[C@H](OC(=O)NS(=O)(=O)NCCOCCOCCO[C@@H]2O[C@H](CO)[C@@H](O)[C@H](O)[C@H]2O)C1]

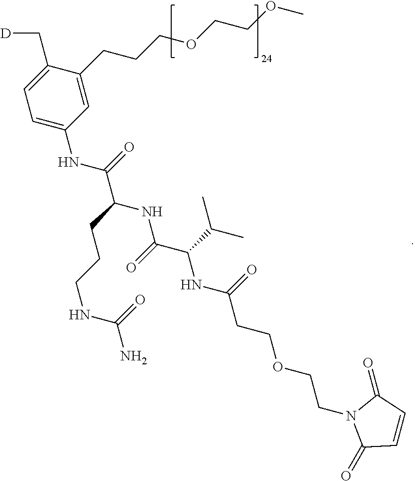

41. The antibody-drug conjugate of any one of claims 1 to 40, wherein D comprises a compound of Formula (Ia): 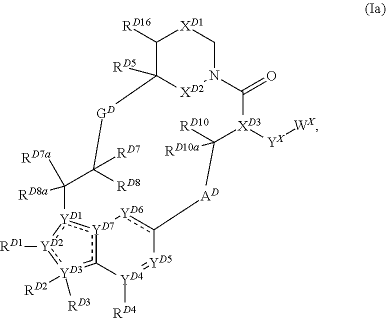
a pharmaceutically acceptable salt thereof, wherein:
the dotted lines represent zero, one, two, three, or four non-adjacent double bonds;
A^{D} is —N(H or CH₃)C(O)—(CH₂)— where the amino nitrogen is bound to the carbon atom of —C(R^{D10a})(R^{D10})—, optionally substituted 3 to 6-membered cycloalkylene, optionally substituted 3 to 6-membered heterocycloalkylene, optionally substituted 6-membered arylene, or optionally substituted 5 to 6-membered heteroarylene;
Y^{X} is 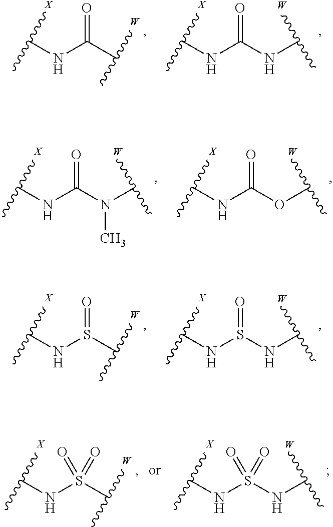
wherein 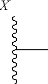
indicates the point that connects with X^{D3}; and 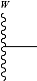
indicates the point that connects with W^{X}; or
y^{X} is —N(R^{D11}—)CO—B^{D}-L^{D}-;
B^{D} is —CH(R^{D9})_or >C═CR^{D9}R^{D9′} where the carbon is bound to the carbonyl carbon of —N(R^{D11})C(O)—, optionally substituted 3 to 6-membered cycloalkylene, optionally substituted 3 to 6-membered heterocycloalkylene, optionally substituted 6-membered arylene, or 5 to 6-membered heteroarylene;
L^{D} is absent or a linker;
G^{D} is optionally substituted C₁-C₄ alkylene, optionally substituted C₁-C₄ alkenylene, optionally substituted C₁-C₄ heteroalkylene, —C(O)O—CH(R^{D6})— where —CH(R^{D6})_is bound to —C(R^{D7}R^{D8})—, —C(O)NH—CH(R^{D6})— where —CH(R^{D6})— is bound to C(R^{D7}R^{D8})—, optionally substituted C₁-C₄ heteroalkylene, or 3 to 8-membered heteroarylene;
W^{X} is hydrogen, cyano, optionally substituted C₁-C₃ heteroalkyl, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₁-C₄ alkyl optionally substituted 3 to 11-membered heterocycloalkyl, optionally substituted 3 to 8-membered cycloalkyl, optionally substituted 6 to 10-membered aryl, or optionally substituted 3 to 8-membered heteroaryl;
X^{D1} is optionally substituted C₁-C₂ alkylene, NR^{D}, O, or S(O)_{nD};
X^{D2} is O or NH;
X^{D3} is N or CH;
nD is 0, 1, or 2;
R^{D} is hydrogen, cyano, optionally substituted C₁-C₄ alkyl, optionally substituted C₂-C₄ alkenyl, optionally substituted C₂-C₄ alkynyl, C(O)R^{D′}, C(O)OR^{D′}, C(O)N(R^{D′})₂, S(O)R^{D′}, S(O)₂R^{D′}, or S(O)₂N(R^{D′})₂; each R^{D′} is, independently, H or optionally substituted C₁-C₄ alkyl;
Y^{D1} is C, CH, or N;
Y^{D2}, Y^{D3}, Y^{D4} and Y^{D7} are, independently, C or N;
Y^{D5} is CH, CH₂, or N;
Y^{D6} is C(O), CH, CH₂, or N;
R^{D1} is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R^{D1} and R^{D2} combine with the atoms to which they are attached to form an optionally substituted 3 to 14-membered heterocycloalkyl;
R^{D2} is absent, hydrogen, optionally substituted C_-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R^{D3} is absent or R^{D2} and R^{D3} combine with the atom to which they are attached to form an optionally substituted 3 to 8-membered cycloalkyl or optionally substituted 3 to 14-membered heterocycloalkyl;
R^{D4} is absent, hydrogen, halogen, cyano, or methyl optionally substituted with 1 to 3 halogens;
R^{D5} is hydrogen, C1-C₄ alkyl optionally substituted with halogen, cyano, hydroxy, or C1-C₄ alkoxy, cyclopropyl, or cyclobutyl;
R^{D6} is hydrogen or methyl;
R^{D7} is hydrogen, halogen, or optionally substituted C_-C₃ alkyl, or
R^{D6} and R^{D7} combine with the carbon atoms to which they are attached to form an optionally substituted 3 to 6-membered cycloalkyl or optionally substituted 3 to 7-membered heterocycloalkyl;
R^{D8} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{D7} and R^{D8} combine with the carbon atom to which they are attached to form C═CR^{D7′}R^{D8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{D7a} and R^{D8a} are, independently, hydrogen, halo, optionally substituted C₁-C₃ alkyl, or combine with the carbon to which they are attached to form a carbonyl;
R^{D7′} is hydrogen, halogen, or optionally substituted C₁-C₃ alkyl;
R^{D8′} is hydrogen, halogen, hydroxyl, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{D7′} and R^{D8′} combine with the carbon atom to which they are attached to form optionally substituted 3 to 6-membered cycloalkyl or optionally substituted 3 to 7-membered heterocycloalkyl;
R^{D9} is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R^{D9} and L^{D} combine with the atoms to which they are attached to form an optionally substituted 3 to 14-membered heterocycloalkyl;
R^{D9′} is hydrogen or optionally substituted C₁-C₆ alkyl;
R^{D10} is hydrogen, halo, hydroxyl, C₁-C₃ alkoxyl, or C₁-C₃ alkyl;
R^{D10a} is hydrogen or halogen;
R^{D11} is hydrogen or C₁-C₃ alkyl; and
R^{D16} is hydrogen or C₁-C₃ alkyl.

42. The antibody-drug conjugate of claim 41, wherein D comprises a compound of Formula (I): 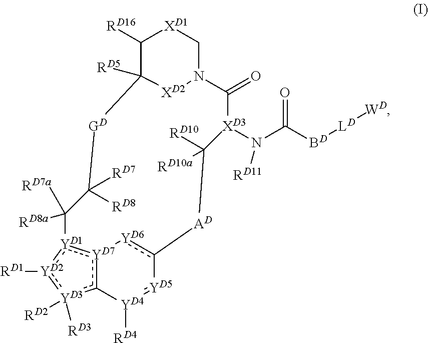
or a pharmaceutically acceptable salt thereof, wherein:
the dotted lines represent zero, one, two, three, or four non-adjacent double bonds;
A^{D} is —N(H or CH₃)C(O)—(CH₂)— where the amino nitrogen is bound to the carbon atom of —C(R^{D10a})(R^{D10})—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B^{D} is —CH(R^{D9})_or >C═CR^{D9}R^{D9′} where the carbon is bound to the carbonyl carbon of —N(R^{D11})C(O)—, optionally substituted 3 to 6-membered cycloalkylene, optionally substituted 3 to 6-membered heterocycloalkylene, optionally substituted 6-membered arylene, or 5 to 6-membered heteroarylene;
G^{D} is optionally substituted C₁-C₄ alkylene, optionally substituted C₁-C₄ alkenylene, optionally substituted C₁-C₄ heteroalkylene, —C(O)O—CH(R^{D6})— where —CH(R^{D6})— is bound to —C(R^{D7}R^{D8})—, —C(O)NH—CH(R^{D6})— where —CH(R^{D6})— is bound to —C(R^{D7}R^{D8})—, optionally substituted C₁-C₄ heteroalkylene, or 3 to 8-membered heteroarylene;
L^{D} is absent or a drug linker;
W^{D} is hydrogen, cyano, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, CO—C4 alkyl optionally substituted 3 to 11-membered heterocycloalkyl, optionally substituted 3 to 8-membered cycloalkyl, optionally substituted 6 to 10-membered aryl, or optionally substituted 3 to 8-membered heteroaryl
X^{D1} is optionally substituted C₁-C₂ alkylene, NR^{D}, O, or S(O)_{nD};
X^{D2} is O or NH;
X^{D3} is N or CH;
nD is 0, 1, or 2;
R^{D} is hydrogen, cyano, optionally substituted C₁-C₄ alkyl, optionally substituted C₂-C₄ alkenyl, optionally substituted C₂-C₄ alkynyl, C(O)R^{D′}, C(O)OR^{D′}, C(O)N(R^{D′})₂, S(O)R^{D′}, S(O)₂R^{D′}, or S(O)₂N(R^{D′})₂; each R^{D′} is, independently, H or optionally substituted C₁-C₄ alkyl;
Y^{D1} is C, CH, or N;
Y^{D2}, Y^{D3}, Y^{D4} and Y^{D7} are, independently, C or N;
Y^{D5} is CH, CH₂, or N;
Y^{D6} is C(O), CH, CH₂, or N;
R^{D1} is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R^{D1} and R^{D2} combine with the atoms to which they are attached to form an optionally substituted 3 to 14-membered heterocycloalkyl;
R^{D2} is absent, hydrogen, optionally substituted C₁-C₆ alkyl, optionally substituted C₂—C alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R^{D3} is absent or R^{D2} and R^{D3} combine with the atom to which they are attached to form an optionally substituted 3 to 8-membered cycloalkyl or optionally substituted 3 to 14-membered heterocycloalkyl;
R^{D4} is absent, hydrogen, halogen, cyano, or methyl optionally substituted with 1 to 3 halogens;
R^{D5} is hydrogen, C₁-C₄ alkyl optionally substituted with halogen, cyano, hydroxy, or C1-C₄ alkoxy, cyclopropyl, or cyclobutyl;
R^{D6} is hydrogen or methyl;
R^{D7} is hydrogen, halogen, or optionally substituted C₁-C₃ alkyl, or
R^{D6} and R^{D7} combine with the carbon atoms to which they are attached to form an optionally substituted 3 to 6-membered cycloalkyl or optionally substituted 3 to 7-membered heterocycloalkyl;
R^{D8} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{D7} and R^{D8} combine with the carbon atom to which they are attached to form C═CR^{D7′}R^{D8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{D7a} and R^{D8a} are, independently, hydrogen, halo, optionally substituted C₁-C₃ alkyl, or combine with the carbon to which they are attached to form a carbonyl;
R^{D7′} is hydrogen, halogen, or optionally substituted C₁-C₃ alkyl;
R^{D8′} is hydrogen, halogen, hydroxyl, cyano, optionally substituted C₁-C₃ alkoxyl, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{D7′} and R^{D8′} combine with the carbon atom to which they are attached to form optionally substituted 3 to 6-membered cycloalkyl or optionally substituted 3 to 7-membered heterocycloalkyl;
R^{D9} is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R^{D9} and L^{D} combine with the atoms to which they are attached to form an optionally substituted 3 to 14-membered heterocycloalkyl;
R^{D9′} is hydrogen or optionally substituted C₁-C₆ alkyl;
R^{D10} is hydrogen, halo, hydroxyl, C₁-C₃ alkoxyl, or C₁-C₃ alkyl;
R^{D10a} is hydrogen or halogen;
R^{D11} is hydrogen or C₁-C₃ alkyl; and
R^{D16} is hydrogen or C₁-C₃ alkyl.

43. The antibody-drug conjugate of claim 41 or 42, wherein D comprises a compound of Formula (Ic): 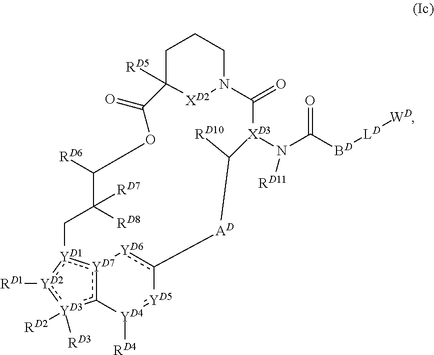
or
a pharmaceutically acceptable salt thereof, wherein
the dotted lines represent zero, one, two, three, or four non-adjacent double bonds;
A^{D} is —N(H or CH₃)C(O)—(CH₂)— where the amino nitrogen is bound to the carbon atom of —CH(R^{D10})—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B^{D} is —CH(R^{D9})— where the carbon is bound to the carbonyl carbon of —N(R^{D11})C(O)—, optionally substituted 3 to 6-membered cycloalkylene, optionally substituted 3 to 6-membered heterocycloalkylene, optionally substituted 6-membered arylene, or 5 to 6-membered heteroarylene;
L^{D} is absent or a drug linker;
W^{D} is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
X^{D2} is O or NH;
X^{D3} is N or CH;
R^{D} is hydrogen, cyano, optionally substituted C₁-C₄ alkyl, optionally substituted C₂-C₄ alkenyl, optionally substituted C₂-C₄ alkynyl, C(O)R^{D′}, C(O)OR^{D′}, C(O)N(R^{D′})₂, S(O)R^{D′}, S(O)₂R^{D′}, or S(O)₂N(R^{D′})₂;
each R^{D′} is, independently, H or optionally substituted C₁-C₄ alkyl;
Y^{D1} is C, CH, or N;
Y^{D2}, Y^{D3}, Y^{D4} and Y^{D7} are, independently, C or N;
Y^{D5} and Y^{D6} are, independently, CH or N;
R^{D1} is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R^{D2} is hydrogen, optionally substituted C₁-C₆ alkyl, optionally substituted C₂-C₆ alkenyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R^{D3} is absent; or
R^{D2} and R^{D3} combine with the atom to which they are attached to form an optionally substituted 3 to 8-membered cycloalkyl or optionally substituted 3 to 14-membered heterocycloalkyl;
R^{D4} is absent, hydrogen, halogen, cyano, or methyl optionally substituted with 1 to 3 halogens;
R^{D5} is hydrogen, C₁-C₄ alkyl optionally substituted with halogen, cyano, hydroxy, or C₁-C₄ alkoxy, cyclopropyl, or cyclobutyl;
R^{D6} is hydrogen or methyl;
R^{D7} is hydrogen, halogen, or optionally substituted C₁-C₃ alkyl, or
R^{D6} and R^{D7} combine with the carbon atoms to which they are attached to form an optionally substituted 3 to 6-membered cycloalkyl or optionally substituted 3 to 7-membered heterocycloalkyl;
R^{D8} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{D7} and R^{D8} combine with the carbon atom to which they are attached to form C═CR^{7′}R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{D7′} is hydrogen, halogen, or optionally substituted C₁-C₃ alkyl;
R^{D8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{D7′} and R^{D8′} combine with the carbon atom to which they are attached to form optionally substituted 3 to 6-membered cycloalkyl or optionally substituted 3 to 7-membered heterocycloalkyl;
R^{D9} is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R^{D10} is hydrogen, hydroxy, 01-O₃ alkoxy, or 01-O₃ alkyl; and
R^{D11} is hydrogen or C₁-C₃ alkyl.

44. The antibody-drug conjugate of any one of claims 41 to 43, wherein D comprises a compound of Formula (If): 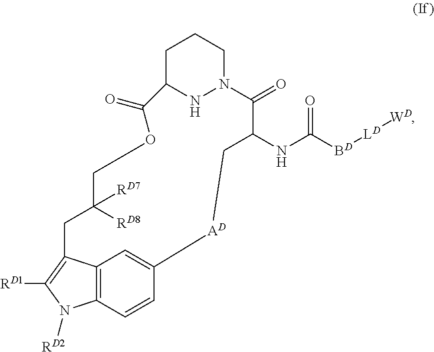
or
a pharmaceutically acceptable salt thereof, wherein
A^{D} is —N(H or CH₃)C(O)—(CH₂)— where the amino nitrogen is bound to the carbon atom of —CH₂—, optionally substituted 3 to 6-membered cycloalkylene, optionally substituted 3 to 6-membered heterocycloalkylene, optionally substituted 6-membered arylene, or optionally substituted 5 to 6-membered heteroarylene;
B^{D} is —CH(R^{D9})— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L^{D} is absent or a drug linker;
W^{D} is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R^{D1} is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl;
R^{D2} is C₁-C₆ alkyl or 3 to 6-membered cycloalkyl;
R^{D7} is C₁-C₃ alkyl;
R^{D8} is C₁-C₃ alkyl; and
R^{D9} is optionally substituted C₁-C₆ alkyl, optionally substituted C₁-C₆ heteroalkyl, optionally substituted 3 to 6-membered cycloalkyl, or optionally substituted 3 to 7-membered heterocycloalkyl.

45. The antibody-drug conjugate of any one of claims 41-44, wherein R^{D}1 is 5 to 10-membered heteroaryl.

46. The antibody-drug conjugate of any one of claims 41-45, wherein R^{D1} is optionally substituted 6-membered aryl or optionally substituted 6-membered heteroaryl.

47. The antibody-drug conjugate of any one of claims 41-46, wherein D is attached to the conjugate linker represented by L at A^{D} or R^{D1} position.

48. The antibody-drug conjugate of any one of claims 41-47, wherein D comprises a compound of formula (Ig): 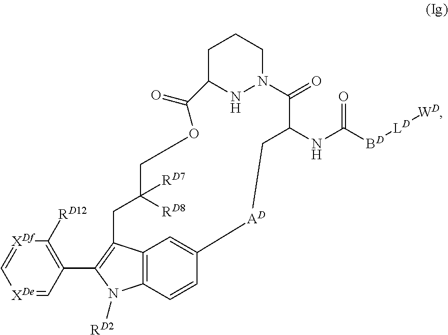
or
a pharmaceutically acceptable salt thereof, wherein:
A^{D} is, optionally substituted 3 to 6-membered cycloalkylene, optionally substituted 3 to 6-membered heterocycloalkylene, optionally substituted 6-membered arylene, or optionally substituted 5 to 6-membered heteroarylene;
B^{D} is —CH(R^{D9})— where the carbon is bound to the carbonyl carbon of —NHC(O)—, optionally substituted 3 to 6-membered cycloalkylene, optionally substituted 3 to 6-membered heterocycloalkylene, optionally substituted 6-membered arylene, or 5 to 6-membered heteroarylene;
L^{D} is absent or a drug linker;
W^{D} is hydrogen, optionally substituted amino, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 8-membered cycloalkyl, or optionally substituted 3 to 8-membered heteroaryl;
R^{D2} is C₁-C₆ alkyl or 3 to 6-membered cycloalkyl;
R^{D7} is C₁-C₃ alkyl;
R^{D8} is C₁-C₃ alkyl;
R^{D9} is optionally substituted C₁-C₆ alkyl, optionally substituted O₁-06 heteroalkyl, optionally substituted 3 to 6-membered cycloalkyl, or optionally substituted 3 to 7-membered heterocycloalkyl;
X^{De} is N, CH, or CR^{D17};
X^{Df} is N or CH;
R^{D12} is optionally substituted C₁-C₆ alkyl or optionally substituted C₁-C₆ heteroalkyl; and
R^{D17} is optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl.

49. The antibody-drug conjugate of any one of claims 41-48, wherein A^{D} is optionally substituted 6-membered arylene.

50. The antibody-drug conjugate of any one of claims 41-49, wherein A^{D} is optionally substituted 5 to 6-membered heteroarylene.

51. The antibody-drug conjugate of any one of claims 41-50, wherein BD is —CHR^{D9}—.

52. The antibody-drug conjugate of any one of claims 41-51, wherein R^{D9} is optionally substituted C₁-C₆ alkyl or optionally substituted 3 to 6-membered cycloalkyl.

53. The antibody-drug conjugate of any one of claims 41-52, wherein the drug linker is the structure of Formula II: 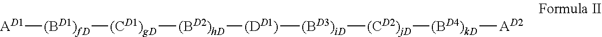
wherein
A^{D1} is a bond between the drug linker and B; A^{D2} is a bond between Wand the drug linker;
B^{D1}, B^{D2}, B^{D3}, and B^{D4} each, independently, is selected from optionally substituted C₁-C₂ alkylene, optionally substituted C₁-C₃ heteroalkylene, O, S, and NR^{DN}; R^{DN} is hydrogen, optionally substituted C₁-C₄ alkyl, optionally substituted C₁-C₃ cycloalkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl;
C^{D1} and C^{D2} are each, independently, selected from carbonyl, thiocarbonyl, sulphonyl, or phosphoryl;
fD, gD, hD, iD, jD, and kD are each, independently, 0 or 1; and
D^{D1} is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A^{D1}-(B^{D1})_{fD}—(C^{D1})_{gD}—(B^{D2})_{hD}— to —(B^{D3})_{iD}—(C^{D2})_{Dj}—(B^{D4})_{Dk}-A^{D2}.

54. The antibody-drug conjugate of any one of claims 41-53, wherein the drug linker has the structure of Formula Ila: 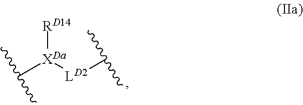
wherein
X^{Da} is absent or N;
R^{D14} is absent, hydrogen, optionally substituted C₁-C₆ alkyl, or optionally substituted C₁-C₃ cycloalkyl; and
L^{D2} is absent, —C(O)—, —SO₂—, optionally substituted C₁-C₄ alkylene or optionally substituted C₁-C₄ heteroalkylene, wherein at least one of X^{Da}, R^{D14}, or L^{D2} is present.

55. The antibody-drug conjugate of any one of claims 41 to 54, wherein W^{D} is hydrogen.

56. The antibody-drug conjugate of any one of claims 41 to 54, wherein W^{D} is C₀-C4 alkyl optionally substituted 3 to 11-membered heterocycloalkyl.

57. The antibody-drug conjugate of any one of claims 46 to 56, wherein D is attached to the conjugate linker represented by L at A^{D} or R^{D17} position.

58. The antibody-drug conjugate of any one of claims 41 to 47, wherein D comprises a compound of formula (Ih): 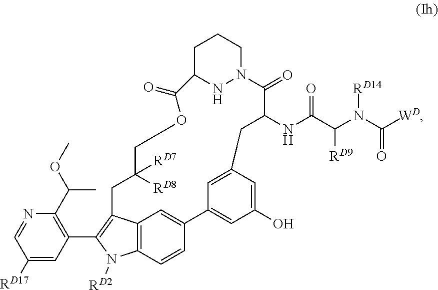
or
a pharmaceutically acceptable salt thereof, wherein:
R^{D2} is C₁-C₃ alkyl;
R^{D7} is C₁-C₃ alkyl;
R^{D8} is C₁₋₃ alkyl;
R^{D9} is C₁-C₆ alkyl;
R^{D14} is hydrogen or C₁-C₆ alkyl,
R^{D17} is optionally substituted 3 to 6-membered cycloalkyl or optionally substituted 3 to 6-membered heterocycloalkyl; and
W^{D} is optionally substituted 3 to 11-membered heterocycloalkyl.

59. The antibody-drug conjugate of claim 58, wherein
R^{D9} is C₁-C₃ alkyl;
R^{D14} is C₁-C₃ alkyl,
R^{D17} is optionally substituted 3 to 6-membered heterocycloalkyl; and
W^{D} is optionally substituted 5 to 6-membered heterocycloalkyl.

60. The antibody-drug conjugate of claim 58 or 59, wherein D comprises a compound represented by 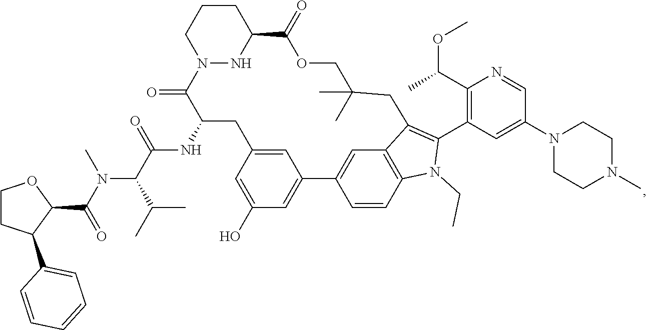 [SMILES: CCn1c(-c2cc(N3CCN(C)CC3)cnc2[C@H](C)OC)c2c3cc(ccc31)-c1cc(O)cc(c1)C[C@H](NC(=O)[C@H](C(C)C)N(C)C(=O)[C@@H]1OCC[C@@H]1c1ccccc1)C(=O)N1CCC[C@H](N1)C(=O)OCC(C)(C)C2]
or
a pharmaceutically acceptable salt thereof.

61. The antibody-drug conjugate of claim 41, wherein D comprises a compound of formula (In): 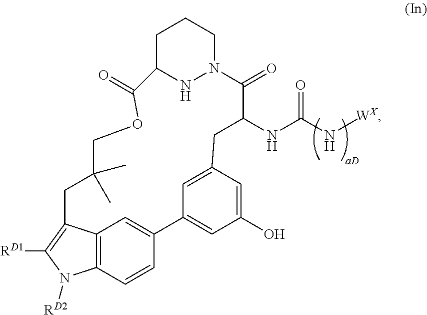
or
a pharmaceutically acceptable salt thereof, wherein aD is 0 or 1.

62. The antibody-drug conjugate of claim 41, wherein D comprises a compound of formula (Ij): 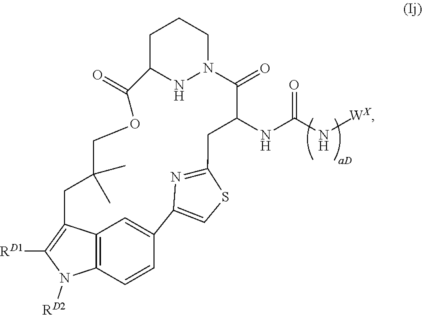
or
a pharmaceutically acceptable salt thereof, wherein aD is 0 or 1.

63. The antibody-drug conjugate of claim 41, wherein D comprises a compound of formula (Ik): 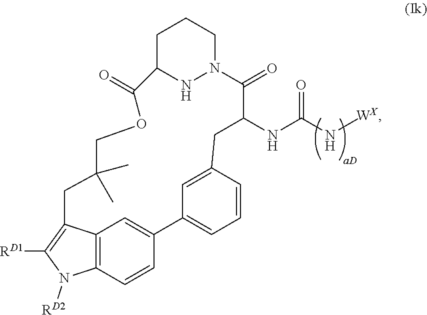
a pharmaceutically acceptable salt thereof, wherein aD is 0 or 1.

64. The antibody-drug conjugate of claim 41, wherein D comprises a compound of formula (Im): 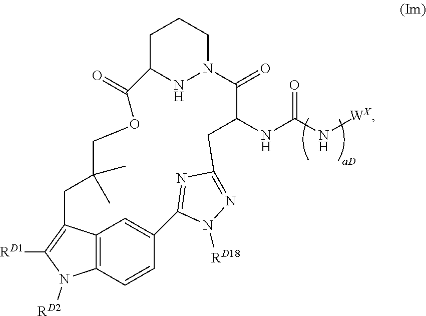
a pharmaceutically acceptable salt thereof, wherein aD is 0 or 1.

65. The antibody-drug conjugate any one of claims 1 to 40, wherein D comprises a group represented by a formula selected from those in Table A2.

66. The antibody-drug conjugate of any one of claims 1 to 65, wherein the antibody or antigen-binding fragment thereof binds to a target antigen on a cancer cell.

67. The antibody-drug conjugate of claim 66, wherein the target antigen is EphA2 or B7-H3 (CD276).

68. The antibody-drug conjugate of any one of claims 1 to 67, wherein the antibody or antigen-binding fragment thereof is an anti-EphA2 antibody or antigen-binding fragment thereof.

69. The antibody-drug conjugate of claim 68, wherein the anti-EphA2 antibody or antigen-binding fragment thereof comprises three heavy chain CDRs and three light chain CDRs selected from the group consisting of:
1) heavy chain CDR1 (HCDR1) consisting of SEQ ID NO:17, heavy chain CDR2 (HCDR2) consisting of SEQ ID NO:18, heavy chain CDR3 (HCDR3) consisting of SEQ ID NO:19; light chain CDR1 (LCDR1) consisting of SEQ ID NO:26, light chain CDR2 (LCDR2) consisting of SEQ ID NO:27, and light chain CDR3 (LCDR3) consisting of SEQ ID NO:28;
2) heavy chain CDR1 (HCDR1) consisting of SEQ ID NO:20, heavy chain CDR2 (HCDR2) consisting of SEQ ID NO:21, heavy chain CDR3 (HCDR3) consisting of SEQ ID NO:19; light chain CDR1 (LCDR1) consisting of SEQ ID NO:29, light chain CDR2 (LCDR2) consisting of SEQ ID NO:30, and light chain CDR3 (LCDR3) consisting of SEQ ID NO:31;
3) heavy chain CDR1 (HCDR1) consisting of SEQ ID NO:22, heavy chain CDR2 (HCDR2) consisting of SEQ ID NO:23, heavy chain CDR3 (HCDR3) consisting of SEQ ID NO:24; light chain CDR1 (LCDR1) consisting of SEQ ID NO:32, light chain CDR2 (LCDR2) consisting of SEQ ID NO:27, and light chain CDR3 (LCDR3) consisting of SEQ ID NO:31; and
4) heavy chain CDR1 (HCDR1) consisting of SEQ ID NO:25, heavy chain CDR2 (HCDR2) consisting of SEQ ID NO:21, heavy chain CDR3 (HCDR3) consisting of SEQ ID NO:19; light chain CDR1 (LCDR1) consisting of SEQ ID NO:29, light chain CDR2 (LCDR2) consisting of SEQ ID NO:30, and light chain CDR3 (LCDR3) consisting of SEQ ID NO:31.

70. The antibody-drug conjugate of claim 68 or 69, wherein the anti-EphA2 antibody or antigen-binding fragment thereof comprises: a heavy chain variable region comprising the amino acid sequence of SEQ ID NO:11, and a light chain variable region comprising the amino acid sequence of SEQ ID NO:12.

71. The antibody-drug conjugate of any one of claims 68 to 70, wherein the anti-EphA2 antibody or antigen-binding fragment thereof comprises: a heavy chain comprising the amino acid sequence of SEQ ID NO:3, and a light chain comprising the amino acid sequence of SEQ ID NO:5.

72. The antibody-drug conjugate of any one of claims 1 to 67, wherein the antibody or antigen-binding fragment thereof is an anti-B7-H3 (CD276) antibody or antigen-binding fragment.

73. The antibody-drug conjugate of claim 72, wherein the anti-B7-H3 (CD276) antibody comprises three heavy chain CDRs and three light chain CDRs selected from the group consisting of:
1) heavy chain CDR1 (HCDR1) consisting of SEQ ID NO:33, heavy chain CDR2 (HCDR2) consisting of SEQ ID NO:34, heavy chain CDR3 (HCDR3) consisting of SEQ ID NO:35; light chain CDR1 (LCDR1) consisting of SEQ ID NO:42, light chain CDR2 (LCDR2) consisting of SEQ ID NO:43, and light chain CDR3 (LCDR3) consisting of SEQ ID NO:44;
2) heavy chain CDR1 (HCDR1) consisting of SEQ ID NO:36, heavy chain CDR2 (HCDR2) consisting of SEQ ID NO:37, heavy chain CDR3 (HCDR3) consisting of SEQ ID NO:35; light chain CDR1 (LCDR1) consisting of SEQ ID NO:45, light chain CDR2 (LCDR2) consisting of SEQ ID NO:46, and light chain CDR3 (LCDR3) consisting of SEQ ID NO:47;
3) heavy chain CDR1 (HCDR1) consisting of SEQ ID NO:38, heavy chain CDR2 (HCDR2) consisting of SEQ ID NO:39, heavy chain CDR3 (HCDR3) consisting of SEQ ID NO:40; light chain CDR1 (LCDR1) consisting of SEQ ID NO:48, light chain CDR2 (LCDR2) consisting of SEQ ID NO:43, and light chain CDR3 (LCDR3) consisting of SEQ ID NO:47;
4) heavy chain CDR1 (HCDR1) consisting of SEQ ID NO:41, heavy chain CDR2 (HCDR2) consisting of SEQ ID NO:37, heavy chain CDR3 (HCDR3) consisting of SEQ ID NO:35; light chain CDR1 (LCDR1) consisting of SEQ ID NO:45, light chain CDR2 (LCDR2) consisting of SEQ ID NO:46, and light chain CDR3 (LCDR3) consisting of SEQ ID NO:47.

74. The antibody-drug conjugate of claim 72 or 73, wherein the anti-B7-H3 (CD276) antibody comprises: a heavy chain variable region comprising the amino acid sequence of SEQ ID NO:13, and a light chain variable region comprising the amino acid sequence of SEQ ID NO:14.

75. The antibody-drug conjugate of any one of claims 72 to 74, wherein the anti-B7-H3 (CD276) antibody comprises: a heavy chain comprising the amino acid sequence of SEQ ID NO:7 and a light chain comprising the amino acid sequence of SEQ ID NO:8.

76. The antibody-drug conjugate of claim 72, wherein the anti-B7-H3 (CD276) antibody comprises three heavy chain CDRs and three light chain CDRs selected from the group consisting of:
1) heavy chain CDR1 (HCDR1) consisting of SEQ ID NO:49, heavy chain CDR2 (HCDR2) consisting of SEQ ID NO:50, heavy chain CDR3 (HCDR3) consisting of SEQ ID NO:51; light chain CDR1 (LCDR1) consisting of SEQ ID NO:58, light chain CDR2 (LCDR2) consisting of SEQ ID NO:59, and light chain CDR3 (LCDR3) consisting of SEQ ID NO:60;
2) heavy chain CDR1 (HCDR1) consisting of SEQ ID NO:52, heavy chain CDR2 (HCDR2) consisting of SEQ ID NO:53, heavy chain CDR3 (HCDR3) consisting of SEQ ID NO:51; light chain CDR1 (LCDR1) consisting of SEQ ID NO:61 light chain CDR2 (LCDR2) consisting of SEQ ID NO:62, and light chain CDR3 (LCDR3) consisting of SEQ ID NO:63:
3) heavy chain CDR1 (HCDR1) consisting of SEQ ID NO:54, heavy chain CDR2 (HCDR2) consisting of SEQ ID NO:55, heavy chain CDR3 (HCDR3) consisting of SEQ ID NO:56; light chain CDR1 (LCDR1) consisting of SEQ ID NO:58, light chain CDR2 (LCDR2) consisting of SEQ ID NO:59, and light chain CDR3 (LCDR3) consisting of SEQ ID NO:63; and
4) heavy chain CDR1 (HCDR1) consisting of SEQ ID NO:57, heavy chain CDR2 (HCDR2) consisting of SEQ ID NO:53, heavy chain CDR3 (HCDR3) consisting of SEQ ID NO:51; light chain CDR1 (LCDR1) consisting of SEQ ID NO:61, light chain CDR2 (LCDR2) consisting of SEQ ID NO:62, and light chain CDR3 (LCDR3) consisting of SEQ ID NO:63.

77. The antibody-drug conjugate of claim 72 or 76, wherein the anti-B7-H3 (CD276) antibody comprises: a heavy chain variable region comprising the amino acid sequence of SEQ ID NO:15, and a light chain variable region comprising the amino acid sequence of SEQ ID NO:16.

78. The antibody-drug conjugate of any one of claims 72, 76 or 77, wherein the anti-B7-H3 (CD276) antibody comprises: a heavy chain comprising the amino acid sequence of SEQ ID NO:9, and the light chain comprising the amino acid sequence of SEQ ID NO:10.

79. The antibody-drug conjugate of any one of claims 1 to 78, wherein:
(a) the antibody or antigen-binding fragment thereof comprises an IgG1 heavy chain constant domain or a modified IgG1 heavy chain constant domain, optionally the IgG1 heavy chain constant domain comprises a cysteine residue (C) at position 152 and position 375, wherein the positions are numbered according to the EU system; and/or
(b) the antibody or antigen-binding fragment thereof comprises an Ig kappa light chain constant domain.

80. A composition comprising multiple copies of the antibody-drug conjugate of any one of claims 1 to 79, wherein the average p of the antibody-drug conjugates in the composition is from about 2 to about 16, e.g., about 2 to about 8, e.g., about 2 to about 4.

81. A pharmaceutical composition comprising the antibody-drug conjugate of any one of claims 1 to 79 or the composition of claim 80, and a pharmaceutically acceptable carrier.

82. A method of treating a subject having or suspected of having a cancer, comprising administering to the subject a therapeutically effective amount of the antibody-drug conjugate of any one of claims 1 to 79, the composition of claim 80, or the pharmaceutical composition of claim 81.

83. The method of claim 82, wherein the cancer expresses a target antigen, optionally wherein the target antigen is EphA2 or B7-H3 (CD276).

84. The method of claim 82 or 83, wherein the cancer is a tumor or a hematological cancer, optionally, the cancer is a breast cancer including ER positive breast cancer, multiple myeloma, plasma cell myeloma, leukemia, lymphoma, sarcoma, gastric or stomach cancer, acute myeloid leukemia, bladder cancer, brain cancer, bone marrow cancer, cervical cancer, chronic lymphocytic leukemia, colorectal cancer, pancreatic cancer, esophageal cancer, hepatocellular cancer, lymphoblastic leukemia including acute lymphoblastic leukemia, follicular lymphoma, lymphoid malignancies of T-cell or B-cell origin, metastatic castration resistant prostate cancer, bladder urothelial carcinoma, melanoma, myelogenous leukemia, myeloma, oral cancer, ovarian cancer, non-small cell lung cancer, prostate cancer, small cell lung cancer, spleen cancer, or head and neck cancer.

85. A method of reducing or inhibiting the growth of a tumor in a subject, comprising administering to the subject a therapeutically effective amount of the antibody-drug conjugate of any one of claims 1 to 79, the composition of claim 80, or the pharmaceutical composition of claim 81.

86. The method of claim 85, wherein the tumor expresses a target antigen, optionally wherein the target antigen is EphA2 or B7-H3 (CD276).

87. The method of claim 85 or 86, wherein the tumor is a breast cancer including ER positive breast cancer, multiple myeloma, plasma cell myeloma, leukemia, lymphoma, sarcoma, gastric or stomach cancer, acute myeloid leukemia, bladder cancer, brain cancer, bone marrow cancer, cervical cancer, chronic lymphocytic leukemia, colorectal cancer, pancreatic cancer, esophageal cancer, hepatocellular cancer, lymphoblastic leukemia including acute lymphoblastic leukemia, follicular lymphoma, lymphoid malignancies of T-cell or B-cell origin, metastatic castration resistant prostate cancer, bladder urothelial carcinoma, melanoma, myelogenous leukemia, myeloma, oral cancer, ovarian cancer, non-small cell lung cancer, prostate cancer, small cell lung cancer, spleen cancer, or head and neck cancer.

88. A method of reducing or inhibiting a hematological cancer in a subject, comprising administering to the subject a therapeutically effective amount of the antibody-drug conjugate of any one of claims 1 to 79, the composition of claim 80, or the pharmaceutical composition of claim 81.

89. The method of claim 88, wherein the hematological cancer expresses a target antigen, optionally wherein the target antigen is EphA2 or B7-H3 (CD276).

90. The method of claim 88 or 89, wherein the hematological cancer is chronic lymphocytic leukemia (CLL), follicular lymphoma, mantle cell lymphoma, diffuse large B-cell lymphoma, acute lymphoblastic leukemia (ALL), acute myeloid leukemia (AML), chronic lymphocytic leukemia (CLL), chronic myelogenous leukemia (CML), chronic myelomonocytic leukemia (CMML), acute monocytic leukemia (AMoL), Hodgkin's lymphoma, non-Hodgkin's lymphoma or myelodysplasia syndrome (MDS).

91. The method of any one of claims 85 to 90, wherein administration of the antibody-drug conjugate, composition, or pharmaceutical composition reduces or inhibits the growth of the tumor or hematological cancer by at least about 10%, at least about 20%, at least about 30%, at least about 40%, at least about 50%, at least about 60%, at least about 70%, at least about 80%, at least about 90%, at least about 95%, or at least about 99%.

92. A method of reducing or slowing the expansion of a cancer cell population in a subject, comprising administering to the subject a therapeutically effective amount of the antibody-drug conjugate of any one of claims 1 to 79, the composition of claim 80, or the pharmaceutical composition of claim 81.

93. The method of claim 92, wherein the cancer cell population expresses a target antigen, optionally wherein the target antigen is EphA2 or B7-H3 (CD276).

94. The method of claim 92 or 93, wherein the cancer cell population is from a tumor or a hematological cancer, optionally wherein the cancer cell population is from a breast cancer including ER positive breast cancer, multiple myeloma, plasma cell myeloma, leukemia, lymphoma, sarcoma, gastric or stomach cancer, acute myeloid leukemia, bladder cancer, brain cancer, bone marrow cancer, cervical cancer, chronic lymphocytic leukemia, colorectal cancer, pancreatic cancer, esophageal cancer, hepatocellular cancer, lymphoblastic leukemia including acute lymphoblastic leukemia, follicular lymphoma, lymphoid malignancies of T-cell or B-cell origin, metastatic castration resistant prostate cancer, bladder urothelial carcinoma, melanoma, myelogenous leukemia, myeloma, oral cancer, ovarian cancer, non-small cell lung cancer, prostate cancer, small cell lung cancer, spleen cancer, or head and neck cancer.

95. The method of any one of claims 92 to 94, wherein administration of the antibody-drug conjugate, composition, or pharmaceutical composition reduces the cancer cell population or slows the expansion of the cancer cell population by at least about 10%, at least about 20%, at least about 30%, at least about 40%, at least about 50%, at least about 60%, at least about 70%, at least about 80%, at least about 90%, at least about 95%, or at least about 99%.

96. The method of any one of claims 82 to 95, wherein the antibody-drug conjugate is administered as monotherapy.

97. The method of any one of claims 82 to 95, wherein the antibody-drug conjugate is administered adjunctive to another therapeutic agent or radiation therapy.

98. The method of claim 97, wherein the antibody-drug conjugate is administered in an amount effective to sensitize the tumor cells to one or more additional therapeutic agents and/or radiation therapy.

99. The method of any one of claims 82 to 95, further comprising administering to the subject in need thereof at least one additional therapeutic agent.

100. A method of inhibiting panRAS activity in a cell that expresses panRAS, comprising contacting the cell with an antibody-drug conjugate of any one of claims 1 to 79 that is capable of binding the cell, under conditions in which the antibody-drug conjugate binds the cell.

101. A method of determining whether a subject having or suspected of having a cancer will be responsive to treatment with the antibody-drug conjugate of any one of claims 1 to 79, the composition of claim 80, or the pharmaceutical composition of claim 81, comprising providing a biological sample from the subject; contacting the sample with the antibody-drug conjugate; and
detecting binding of the antibody-drug conjugate to cancer cells in the sample.

102. The method of claim 101, wherein the cancer cells in the sample express a target antigen, optionally wherein the target antigen is EphA2 or B7-H3 (CD276).

103. The method of claim 101 or claim 102, wherein the cancer expresses a target antigen, optionally wherein the target antigen is EphA2 or B7-H3 (CD276).

104. The method of any one of claims 101 to 103, wherein the cancer is a tumor or a hematological cancer, optionally the cancer is a breast cancer including ER positive breast cancer, multiple myeloma, plasma cell myeloma, leukemia, lymphoma, sarcoma, gastric or stomach cancer, acute myeloid leukemia, bladder cancer, brain cancer, bone marrow cancer, cervical cancer, chronic lymphocytic leukemia, colorectal cancer, pancreatic cancer, esophageal cancer, hepatocellular cancer, lymphoblastic leukemia including acute lymphoblastic leukemia, follicular lymphoma, lymphoid malignancies of T-cell or B-cell origin, metastatic castration resistant prostate cancer, bladder urothelial carcinoma, melanoma, myelogenous leukemia, myeloma, oral cancer, ovarian cancer, non-small cell lung cancer, prostate cancer, small cell lung cancer, spleen cancer, or head and neck cancer.

105. The method of any one of claims 101 to 104, wherein the sample is a tissue biopsy sample, a blood sample, or a bone marrow sample.

106. The method of any one of claims 83 to 105, wherein the target antigen is EphA2.

107. The method of any one of claims 83 to 105, wherein the target antigen is B7-H3 (CD276).

108. A method of producing the antibody-drug conjugate of any one of claims 1 to 80, comprising reacting an antibody or antigen-binding fragment with a cleavable linker joined to a panRAS inhibitor under conditions that allow conjugation.

109. The method of claim 108, wherein the antibody or antigen-binding fragment is an anti-EphA2 antibody or antigen-binding fragment or a B7-H3 (CD276) antibody or antigen-binding fragment.

110. The method of claim 109, wherein the antibody or antigen-binding fragment is an anti-EphA2 antibody or antigen-binding fragment.

111. The method of claim 109, wherein the antibody or antigen-binding fragment is a B7-H3 (CD276) antibody or antigen-binding fragment.

112. Use of the antibody-drug conjugate of any one of claims 1 to 79, the composition of claim 80, or the pharmaceutical composition of claim 81, for manufacture of a medicament for treating a subject having or suspected of having a cancer.

113. Use of the antibody-drug conjugate of any one of claims 1 to 79, the composition of claim 80, or the pharmaceutical composition of claim 81, for manufacture of a medicament for reducing or inhibiting the growth of a tumor in a subject.

114. Use of the antibody-drug conjugate of any one of claims 1 to 79, the composition of claim 80, or the pharmaceutical composition of claim 81, for manufacture of a medicament for reducing or inhibiting a hematological cancer in a subject.

115. Use of the antibody-drug conjugate of any one of claims 1 to 79, the composition of claim 80, or the pharmaceutical composition of claim 81, for manufacture of a medicament for reducing or slowing the expansion of a cancer cell population in a subject.


SEQUENCE LISTING (74 sequences)

SEQ ID NO: 1 - protein, 976 aa, unidentified
MELQAARACFALLWGCALAAAAAAQGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNLAVIGGVAVGVVLLLVLAGVGFFIHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPI

SEQ ID NO: 2 - protein, 534 aa, unidentified
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 3 - protein, 458 aa, unidentified
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 4 - DNA, 1374 bp, synthetic construct
  source 1..1374: mol_type = other DNA
gaagttcagctgcttgaatctggcggcggactggttcaacctggcggatctctgagactgagctgtgccgccagcggcttcaccttcagccactatatgatggcctgggtccgacaggcccctggcaaaggacttgaatgggtgtccagaatcggcccctctggcggccctacacactacgctgattctgtgaagggcagattcaccatcagccgggacaacagcaagaacaccctgtacctgcagatgaacagcctgagagccgaggacaccgccgtgtattactgtgccggctacgacagcggctacgattatgtggctgtggccggacctgccgagtactttcagcattggggacagggcaccctggtcaccgttagttctgctagcaccaagggcccaagtgtgtttcccctggcccccagcagcaagtctacttccggcggaactgctgccctgggttgcctggtgaaggactacttcccctgtcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctacccctgtgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 5 - protein, 214 aa, synthetic construct
DIQMTQSPSSLSASVGDRVTITCRASQSISTWLAWYQQKPGKAPKLLIYKASNLHTGVPSRFSGSGSGTEFSLTISGLQPDDFATYYCQQYNSYSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - DNA, 642 bp, synthetic construct
  source 1..642: mol_type = other DNA
gacattcagatgacacagagccctagcagcctgagcgcctctgtgggagacagagtgaccatcacctgtagagccagccagagcatcagcacatggctggcctggtatcagcagaagcctggcaaggcccctaagctgctgatctacaaggccagcaatctgcacaccggcgtgcccagcagattttctggctctggaagcggcaccgagttcagcctgacaatctctggcctgcagcctgacgacttcgccacctactactgccagcagtacaacagctacagccggacctttggccagggaacaaaggtggaaatcaagcgtacggtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 7 - protein, 447 aa, synthetic construct
EVQLVQSGAEVKKPGSSVKVSCKASGYTFSSYWMHWVRQAPGQGLEWIGLIHPESGSTNYNEMFKNRATLTVDRSTSTAYMELSSLRSEDTAVYYCAGGGRLYFDYWGQGTTVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 8 - protein, 219 aa, synthetic construct
DIVMTQSPLSLPVTPGEPASISCRSSQSLVHSNRDTYLRWYLQKPGQSPQLLIYKVSNRFSGVPDRFSGSGSGTDFTLKISRVEAEDVGVYYCSQSTHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 452 aa, synthetic construct
QVQLVQSGAEVKKPGSSVKVSCKASGYTFTNYVMHWVRQAPGQGLEWMGYINPYNDDVKYNEKFKGRVTITADESTSTAYMELSSLRSEDTAVYYCARWGYYGSPLYYFDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPCDIAVEWESNGQPENNYKTTPPVLDSDGSFFLYSKLTVDKSRWQQGNVFSCSVMHEALHNHYTQKSLSLSPGK

SEQ ID NO: 10 - protein, 213 aa, synthetic construct
EIVLTQSPATLSLSPGERATLSCRASSRLIYMHWYQQKPGQAPRPLIYATSNLASGIPARFSGSGSGTDFTLTISSLEPEDFAVYYCQQWNSNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 128 aa, synthetic construct
EVQLLESGGGLVQPGGSLRLSCAASGFTFSHYMMAWVRQAPGKGLEWVSRIGPSGGPTHYADSVKGRFTISRDNSKNTLYLQMNSLRAEDTAVYYCAGYDSGYDYVAVAGPAEYFQHWGQGTLVTVSS

SEQ ID NO: 12 - protein, 107 aa, synthetic construct
DIQMTQSPSSLSASVGDRVTITCRASQSISTWLAWYQQKPGKAPKLLIYKASNLHTGVPSRFSGSGSGTEFSLTISGLQPDDFATYYCQQYNSYSRTFGQGTKVEIK

SEQ ID NO: 13 - protein, 117 aa, synthetic construct
EVQLVQSGAEVKKPGSSVKVSCKASGYTFSSYWMHWVRQAPGQGLEWIGLIHPESGSTNYNEMFKNRATLTVDRSTSTAYMELSSLRSEDTAVYYCAGGGRLYFDYWGQGTTVTVSS

SEQ ID NO: 14 - protein, 112 aa, synthetic construct
DIVMTQSPLSLPVTPGEPASISCRSSQSLVHSNRDTYLRWYLQKPGQSPQLLIYKVSNRFSGVPDRFSGSGSGTDFTLKISRVEAEDVGVYYCSQSTHVPYTFGGGTKVEIK

SEQ ID NO: 15 - protein, 122 aa, synthetic construct
QVQLVQSGAEVKKPGSSVKVSCKASGYTFTNYVMHWVRQAPGQGLEWMGYINPYNDDVKYNEKFKGRVTITADESTSTAYMELSSLRSEDTAVYYCARWGYYGSPLYYFDYWGQGTLVTVSS

SEQ ID NO: 16 - protein, 106 aa, synthetic construct
EIVLTQSPATLSLSPGERATLSCRASSRLIYMHWYQQKPGQAPRPLIYATSNLASGIPARFSGSGSGTDFTLTISSLEPEDFAVYYCQQWNSNPPTFGQGTKVEIK

SEQ ID NO: 17 - protein, 7 aa, synthetic construct
GFTFSHY

SEQ ID NO: 18 - protein, 6 aa, synthetic construct
GPSGGP

SEQ ID NO: 19 - protein, 19 aa, synthetic construct
YDSGYDYVAVAGPAEYFQH

SEQ ID NO: 20 - protein, 5 aa, synthetic construct
HYMMA

SEQ ID NO: 21 - protein, 17 aa, synthetic construct
RIGPSGGPTHYADSVKG

SEQ ID NO: 22 - protein, 8 aa, synthetic construct
GFTFSHYM

SEQ ID NO: 23 - protein, 8 aa, synthetic construct
IGPSGGPT

SEQ ID NO: 24 - protein, 21 aa, synthetic construct
AGYDSGYDYVAVAGPAEYFQH

SEQ ID NO: 25 - protein, 10 aa, synthetic construct
GFTFSHYMMA

SEQ ID NO: 26 - protein, 7 aa, synthetic construct
SQSISTW

SEQ ID NO: 27 - skipped

SEQ ID NO: 28 - protein, 6 aa, synthetic construct
YNSYSR

SEQ ID NO: 29 - protein, 11 aa, synthetic construct
RASQSISTWLA

SEQ ID NO: 30 - protein, 7 aa, synthetic construct
KASNLHT

SEQ ID NO: 31 - protein, 9 aa, synthetic construct
QQYNSYSRT

SEQ ID NO: 32 - protein, 6 aa, synthetic construct
QSISTW

SEQ ID NO: 33 - protein, 7 aa, synthetic construct
GYTFSSY

SEQ ID NO: 34 - protein, 6 aa, synthetic construct
HPESGS

SEQ ID NO: 35 - protein, 8 aa, synthetic construct
GGRLYFDY

SEQ ID NO: 36 - protein, 5 aa, synthetic construct
SYWMH

SEQ ID NO: 37 - protein, 17 aa, synthetic construct
LIHPESGSTNYNEMFKN

SEQ ID NO: 38 - protein, 8 aa, synthetic construct
GYTFSSYW

SEQ ID NO: 39 - protein, 8 aa, synthetic construct
IHPESGST

SEQ ID NO: 40 - protein, 10 aa, synthetic construct
AGGGRLYFDY

SEQ ID NO: 41 - protein, 10 aa, synthetic construct
GYTFSSYWMH

SEQ ID NO: 42 - protein, 12 aa, synthetic construct
SQSLVHSNRDTY

SEQ ID NO: 43 - skipped

SEQ ID NO: 44 - protein, 6 aa, synthetic construct
STHVPY

SEQ ID NO: 45 - protein, 16 aa, synthetic construct
RSSQSLVHSNRDTYLR

SEQ ID NO: 46 - protein, 7 aa, synthetic construct
KVSNRFS

SEQ ID NO: 47 - protein, 9 aa, synthetic construct
SQSTHVPYT

SEQ ID NO: 48 - protein, 11 aa, synthetic construct
QSLVHSNRDTY

SEQ ID NO: 49 - protein, 7 aa, synthetic construct
GYTFTNY

SEQ ID NO: 50 - protein, 6 aa, synthetic construct
NPYNDD

SEQ ID NO: 51 - protein, 13 aa, synthetic construct
WGYYGSPLYYFDY

SEQ ID NO: 52 - protein, 5 aa, synthetic construct
NYVMH

SEQ ID NO: 53 - protein, 17 aa, synthetic construct
YINPYNDDVKYNEKFKG

SEQ ID NO: 54 - protein, 8 aa, synthetic construct
GYTFTNYV

SEQ ID NO: 55 - protein, 8 aa, synthetic construct
INPYNDDV

SEQ ID NO: 56 - protein, 15 aa, synthetic construct
ARWGYYGSPLYYFDY

SEQ ID NO: 57 - protein, 10 aa, synthetic construct
GYTFTNYVMH

SEQ ID NO: 58 - protein, 6 aa, synthetic construct
SSRLIY

SEQ ID NO: 59 - skipped

SEQ ID NO: 60 - protein, 6 aa, synthetic construct
WNSNPP

SEQ ID NO: 61 - protein, 10 aa, synthetic construct
RASSRLIYMH

SEQ ID NO: 62 - protein, 7 aa, synthetic construct
ATSNLAS

SEQ ID NO: 63 - protein, 9 aa, synthetic construct
QQWNSNPPT

SEQ ID NO: 64 - protein, 189 aa, Homo sapiens [sequence omitted]

SEQ ID NO: 65 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 66 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 67 - protein, 4 aa, synthetic construct
GFLG

SEQ ID NO: 68 - protein, 4 aa, synthetic construct
GLFG

SEQ ID NO: 69 - protein, 4 aa, synthetic construct
ALAL

SEQ ID NO: 70 - protein, 4 aa, synthetic construct
GGGG

SEQ ID NO: 71 - protein, 4 aa, synthetic construct
GFVG

SEQ ID NO: 72 - protein, 4 aa, synthetic construct
GVFG

SEQ ID NO: 73 - protein, 5 aa, synthetic construct
SRLIY

SEQ ID NO: 74 - protein, 4 aa, synthetic construct
  VARIANT 1: note = Residue modified on the terminal end by a spacer, a bond, triazolyl, or CH2-triazolyl
EEEE